(19) United States
(12) Patent Application Publication
(10) Pub. No.: US 2025/0319177 A1
(43) Pub. Date: Oct. 16, 2025

(54) RNA COMPOSITIONS FOR DELIVERY OF MONKEYPOX ANTIGENS AND RELATED METHODS

(71) Applicant: BioNTech SE, Mainz (DE)

(72) Inventors: Asaf Poran, Stoneham, MA (US); Adam Zuiani, Boston, MA (US); Charles Lefco Dulberger, Cambridge, MA (US); Gavin Palowitch, Holbrook, MA (US); John Srouji, Danville, CA (US); Huitian Diao, Boston, MA (US); Daniel Abram Rothenberg, Melrose, MA (US); Ugur Sahin, Mainz (DE); Nilushi S. De Silva, Cambridge, MA (US); Anja Dokic, Mainz (DE); Ricardo Sanchez Velazquez, Mainz (DE); Katja Margit Schlatterer, Mainz (DE); Lynda Maria Stuart, Seattle, WA (US); Alexandra Walls, Cambridge, MA (US)

(21) Appl. No.: 18/869,125

(22) PCT Filed: May 25, 2023
(86) PCT No.: PCT/US23/23632

§ 371 Date: Nov. 25, 2024

Related U.S. Application Data
(60) Provisional application No. 63/345,795, filed on May 25, 2022.
(60) Provisional application No. 63/442,109, filed on Jan. 30, 2023.

(51) Int. Cl. A61K 39/275; A61K 39/00; A61P 31/20; C07K 14/005

(52) U.S. Cl. CPC A61K 39/275; A61P 31/20; C07K 14/005; A61K 2039/53; A61K 2039/55555; A61K 2039/70

(57) ABSTRACT

The present disclosure provides pharmaceutical compositions for delivery of monkeypox antigens (e.g., a monkeypox vaccine) and related technologies (e.g., components thereof and/or methods relating thereto). For example, the present disclosure provides polyribonucleotides encoding one or more monkeypox antigens or fragments thereof.

### CROSS-REFERENCE TO RELATED APPLICATION

The present application claims priority to U.S. Provisional Application No. 63/345,795, filed May 25, 2022, and U.S. Provisional Application No. 63/442,109, filed Jan. 30, 2023, the entireties of which are incorporated herein by reference.

### BACKGROUND

Orthopoxvirus is a genus encompassing a number of viral species including monkeypox virus, vaccinia virus, cowpox virus and variola virus. Some orthopoxviruses are restricted in the hosts they infect, while others have been identified in a broad range of host species. Orthopoxvirues share a number of biological phenotypes including: a lack of a specific receptor required for infection of mammalian cells, a relatively low mutations rate, environmental stability of virion, and the ability to infect hosts via a number of routes (e.g., mucosal, respiratory, parenteral, etc.).

Monkeypox (also referred to herein as mpox) was first discovered in 1958 when two outbreaks of a pox-like disease occurred in colonies of monkeys kept for research, hence the name ‘monkeypox.’ The first human case of monkeypox was recorded in 1970 in the Democratic Republic of Congo during a period of intensified effort to eliminate smallpox. Since then monkeypox has been reported in humans in other central and western African countries. Recently, monkeypox infections have been confirmed in European countries, as well as the US, Canada and Australia.

### SUMMARY

The present disclosure provides pharmaceutical compositions (e.g., immunogenic compositions, e.g., vaccines) for delivering particular monkeypox antigen constructs to a subject (e.g., a patient) and related technologies (e.g., methods). In particular, the present disclosure provides monkeypox vaccine compositions and related technologies (e.g., methods). The present disclosure includes the unexpected discovery that monkeypox antigens, and fragments thereof, provided herein are particularly advantageous for use in preventing or treating monkeypox. The present disclosure includes the recognition than a monkeypox antigen construct (e.g., a monkeypox antigen construct that is or includes a polyribonucleotide) can include one or more B cell antigens or fragments thereof, one or more T cell antigens or fragments thereof, or a combination of B cell antigens or fragments thereof and T cell antigens or fragments thereof.

In some embodiments, the present disclosure provides certain monkeypox antigen constructs particularly useful in effective vaccination. In some embodiments, provided monkeypox antigen constructs are effective for vaccination against monkeypox. In various embodiments, a monkeypox antigen construct includes and/or encodes one or more monkeypox antigens or fragments thereof (e.g., one or more B cell antigens for monkeypox and/or one or more T cell antigens for monkeypox, or fragments thereof). As disclosed herein, T cell antigens include, e.g., CD4 T cell antigens and/or CD8 T cells. For the avoidance of doubt, as will be appreciated by those of skill in the art, any reference herein to an antigen as a “B cell antigen” or “T cell antigen” or the like does not exclude that any given antigen, or any given agent when exposed to an immune system, can activate, induce, and/or cause a diversity of immunological responses that can include, regardless of labels applied for expediency of description, one or both of a B cell response and a T cell response.

The present disclosure also provides the insight that a monkeypox vaccine may cross-protect against other orthopoxviruses, such as, e.g., variola virus. In some embodiments, provided monkeypox antigen constructs are effective for vaccination against monkeypox and one or more other orthopox viruses. In some embodiments, provided monkeypox antigen constructs are effective for vaccination against monkeypox and variola virus. In some embodiments, provided monkeypox antigen constructs are effective for vaccination against monkeypox and a novel orthopox virus.

In some embodiments, a monkeypox antigen construct can include and/or encode at least one of A29L, A35R, B6R, M1R, E8L, A28L, H3L, A45L, B9R, B16R, C10L, C21L, E7R, F3L, F4L, G6R, H5R, I3L, O2L, Q1L, B12R, and/or C17L or fragments thereof. In some embodiments, a monkeypox antigen construct can include and/or encode at least one of A29L, A35R, B6R, M1R, E8L, A28L, and/or H3L or fragments thereof. In some embodiments, a monkeypox antigen construct can include and/or encode one or more antigens selected from: B6R, A35R, M1R, H3L, E8L, and fragments of any thereof. In some embodiments, a monkeypox antigen construct can include and/or encode at least one of A45L, B9R, B16R, C10L, C21L, E7R, F3L, F4L, G6R, H5R, I3L, O2L, Q1L, B12R, and/or C17L or fragments thereof.

In some embodiments, a monkeypox antigen construct can include and/or encode at least one B cell antigen for monkeypox selected from A29L, A35R, B6R, M1R, E8L, A28L, and/or H3L or fragments thereof. In some embodiments, a monkeypox antigen construct can include and/or encode one or more B cell antigens for monkeypox selected from B6R, A35R, M1R, H3L, E8L, and fragments of any thereof.

In some embodiments, a monkeypox antigen construct can include and/or encode at least one intracellular mature virus (IMV) antigen (e.g., IMV-specific antigen). In some embodiments, one or more IMV antigens (e.g., IMV-specific antigens) are selected from H3L, E8L, M1R, A29L, and fragments of any thereof. In some embodiments, a monkeypox antigen construct can include and/or encode at least one extracellular-enveloped virus (EEV) antigen (e.g., EEV-specific antigen). In some embodiments, one or more EEV antigens (e.g., EEV-specific antigens) are selected from A35R, B6R, and fragments thereof. In some embodiments, a monkeypox antigen construct can include and/or encode at least one IMV antigen (e.g., IMV-specific antigen) and at least one EEV antigen (e.g., EEV-specific antigen).

In some embodiments, a monkeypox antigen construct can include and/or encode at least one T cell antigen (e.g., at least one CD4 and/or CD8 T cell antigen) for monkeypox selected from A45L, B9R, B16R, C10L, C21L, E7R, F3L, F4L, G6R, H5R, I3L, O2L, Q1L, B12R, and/or C17L or fragments thereof.

In some embodiments, a monkeypox antigen construct can include and/or encode one or more of (e.g., 1, 2, 3, 4, 5, 6, 7, 8, 9, 10, 11, 12, 13, 14, 15, 16, 17, 18, 19, 20, 21, or 22 of) A29L, A35R, B6R, M1R, E8L, A28L, H3L, A45L, B9R, B16R, C10L, C21L, E7R, F3L, F4L, G6R, H5R, I3L, O2L, Q1L, B12R, and/or C17L or fragments thereof. In some embodiments, a monkeypox antigen construct can include and/or encode one or more (e.g., 1, 2, 3, 4, 5, 6, or 7) of A29L, A35R, B6R, M1R, E8L, A28L, and/or H3L or fragments thereof. In some embodiments, a monkeypox antigen construct can include and/or encode one or more of (e.g., 1, 2, 3, 4, 5, 6, 7, 8, 9, 10, 11, 12, 13, 14, or 15 of) A45L, B9R, B16R, C10L, C21L, E7R, F3L, F4L, G6R, H5R, I3L, O2L, Q1L, B12R, and/or C17L fragments thereof.

In some embodiments, a monkeypox antigen (e.g., a B cell antigen for monkeypox) is or includes an E8L polypeptide or fragment thereof. In various embodiments, an E8L polypeptide or fragment thereof has at least 80% sequence identity with an E8L amino acid sequence set forth in any one of SEQ ID NOs.: 41-50, or otherwise known in the art, or a corresponding portion thereof (e.g., at least 80%, at least 85%, at least 90%, at least 91%, at least 92%, at least 93%, at least 94%, at least 95%, at least 96%, at least 97%, at least 98%, at least 99%, or 100% sequence identity).

In some embodiments, a monkeypox antigen (e.g., a B cell antigen for monkeypox) is or includes an A35R polypeptide or fragment thereof. In various embodiments, an A35R polypeptide or fragment thereof has at least 80% sequence identity with an A35R amino acid sequence set forth in any one of SEQ ID NOs.: 11-20, or otherwise known in the art, or a corresponding portion thereof (e.g., at least 80%, at least 85%, at least 90%, at least 91%, at least 92%, at least 93%, at least 94%, at least 95%, at least 96%, at least 97%, at least 98%, at least 99%, or 100% sequence identity).

In some embodiments, a monkeypox antigen (e.g., a B cell antigen for monkeypox) is or includes a B6R polypeptide or fragment thereof. In various embodiments, a B6R polypeptide or fragment thereof has at least 80% sequence identity with a B6R amino acid sequence set forth in any one of SEQ ID NOs.: 21-30, or otherwise known in the art, or a corresponding portion thereof (e.g., at least 80%, at least 85%, at least 90%, at least 91%, at least 92%, at least 93%, at least 94%, at least 95%, at least 96%, at least 97%, at least 98%, at least 99%, or 100% sequence identity).

In some embodiments, a monkeypox antigen (e.g., a B cell antigen for monkeypox) is or includes a M1R polypeptide or fragment thereof. In various embodiments, a M1R polypeptide or fragment thereof has at least 80% sequence identity with a M1R amino acid sequence set forth in any one of SEQ ID NOs.: 31-40, or otherwise known in the art, or a corresponding portion thereof (e.g., at least 80%, at least 85%, at least 90%, at least 91%, at least 92%, at least 93%, at least 94%, at least 95%, at least 96%, at least 97%, at least 98%, at least 99%, or 100% sequence identity).

In some embodiments, a monkeypox antigen (e.g., a B cell antigen for monkeypox) is or includes a H3L polypeptide or fragment thereof. In various embodiments, a H3L polypeptide or fragment thereof has at least 80% sequence identity with a H3L amino acid sequence set forth in any one of SEQ ID NOs.: 51-60, or otherwise known in the art, or a corresponding portion thereof (e.g., at least 80%, at least 85%, at least 90%, at least 91%, at least 92%, at least 93%, at least 94%, at least 95%, at least 96%, at least 97%, at least 98%, at least 99%, or 100% sequence identity).

In some embodiments, a monkeypox antigen (e.g., a B cell antigen for monkeypox) is or includes an A28L polypeptide or fragment thereof. In various embodiments, an A28L polypeptide or fragment thereof has at least 80% sequence identity with an A28L amino acid sequence set forth in SEQ ID NO.: 196, or otherwise known in the art, or a corresponding portion thereof (e.g., at least 80%, at least 85%, at least 90%, at least 91%, at least 92%, at least 93%, at least 94%, at least 95%, at least 96%, at least 97%, at least 98%, at least 99%, or 100% sequence identity).

In some embodiments, a monkeypox antigen (e.g., a T cell antigen for monkeypox) is or includes an A45L polypeptide or fragment thereof. In various embodiments, an A45L polypeptide or fragment thereof has at least 80% sequence identity with an A45L amino acid sequence set forth in Table 2 or otherwise known in the art, or a corresponding portion thereof (e.g., at least 80%, at least 85%, at least 90%, at least 91%, at least 92%, at least 93%, at least 94%, at least 95%, at least 96%, at least 97%, at least 98%, at least 99%, or 100% sequence identity).

In some embodiments, a monkeypox antigen (e.g., a B cell antigen for monkeypox) is or includes an A29L polypeptide or fragment thereof. In various embodiments, an A29L polypeptide or fragment thereof has at least 80% sequence identity with an A29L amino acid sequence set forth in any one of SEQ ID NOs.: 1-10 or otherwise known in the art, or a corresponding portion thereof (e.g., at least 80%, at least 85%, at least 90%, at least 91%, at least 92%, at least 93%, at least 94%, at least 95%, at least 96%, at least 97%, at least 98%, at least 99%, or 100% sequence identity).

In some embodiments, a monkeypox antigen (e.g., a T cell antigen for monkeypox) is or includes a B9R polypeptide or fragment thereof. In various embodiments, a B9R polypeptide or fragment thereof has at least 80% sequence identity with a B9R amino acid sequence set forth in Table 2 or otherwise known in the art, or a corresponding portion thereof (e.g., at least 80%, at least 85%, at least 90%, at least 91%, at least 92%, at least 93%, at least 94%, at least 95%, at least 96%, at least 97%, at least 98%, at least 99%, or 100% sequence identity).

In some embodiments, a monkeypox antigen (e.g., a T cell antigen for monkeypox) is or includes a B16R polypeptide or fragment thereof. In various embodiments, a B16R polypeptide or fragment thereof has at least 80% sequence identity with a B16R amino acid sequence set forth in Table 2 or otherwise known in the art, or a corresponding portion thereof (e.g., at least 80%, at least 85%, at least 90%, at least 91%, at least 92%, at least 93%, at least 94%, at least 95%, at least 96%, at least 97%, at least 98% at least 99%, or 100% sequence identity).

In some embodiments, a monkeypox antigen (e.g., a T cell antigen for monkeypox) is or includes a C10L polypeptide or fragment thereof. In various embodiments, a C10L polypeptide or fragment thereof has at least 80% sequence identity with a C10L amino acid sequence set forth in Table 2 or otherwise known in the art, or a corresponding portion thereof (e.g., at least 80%, at least 85%, at least 90%, at least 91%, at least 92%, at least 93%, at least 94%, at least 95%, at least 96%, at least 97%, at least 98%, at least 99%, or 100% sequence identity).

In some embodiments, a monkeypox antigen (e.g., a T cell antigen for monkeypox) is or includes a C21L polypeptide or fragment thereof. In various embodiments, a C21L polypeptide or fragment thereof has at least 80% sequence identity with a C21L amino acid sequence set forth in Table 2 or otherwise known in the art, or a corresponding portion thereof (e.g., at least 80%, at least 85%, at least 90%, at least 91%, at least 92%, at least 93%, at least 94%, at least 95%, at least 96%, at least 97%, at least 98%, at least 99%, or 100% sequence identity).

In some embodiments, a monkeypox antigen (e.g., a T cell antigen for monkeypox) is or includes an E7R polypeptide or fragment thereof. In various embodiments, an E7R polypeptide or fragment thereof has at least 80% sequence identity with an E7R amino acid sequence set forth in Table 2 or otherwise known in the art, or a corresponding portion thereof (e.g., at least 80%, at least 85%, at least 90%, at least 91%, at least 92%, at least 93%, at least 94%, at least 95%, at least 96%, at least 97%, at least 98%, at least 99%, or 100% sequence identity).

In some embodiments, a monkeypox antigen (e.g., a T cell antigen for monkeypox) is or includes a F3L polypeptide or fragment thereof. In various embodiments, a F3L polypeptide or fragment thereof has at least 80% sequence identity with a F3L amino acid sequence set forth in Table 2 or otherwise known in the art, or a corresponding portion thereof (e.g., at least 80%, at least 85%, at least 90%, at least 91%, at least 92%, at least 93%, at least 94%, at least 95%, at least 96%, at least 97%, at least 98%, at least 99%, or 100% sequence identity).

In some embodiments, a monkeypox antigen (e.g., a T cell antigen for monkeypox) is or includes a F4L polypeptide or fragment thereof. In various embodiments, a F4L polypeptide or fragment thereof has at least 80% sequence identity with a F4L amino acid sequence set forth in Table 2 or otherwise known in the art, or a corresponding portion thereof (e.g., at least 80%, at least 85%, at least 90%, at least 91%, at least 92%, at least 93%, at least 94%, at least 95%, at least 96%, at least 97%, at least 98%, at least 99%, or 100% sequence identity).

In some embodiments, a monkeypox antigen (e.g., a T cell antigen for monkeypox) is or includes a G6R polypeptide or fragment thereof. In various embodiments, a G6R polypeptide or fragment thereof has at least 80% sequence identity with a G6R amino acid sequence set forth in Table 2 or otherwise known in the art, or a corresponding portion thereof (e.g., at least 80%, at least 85%, at least 90%, at least 91%, at least 92%, at least 93%, at least 94%, at least 95%, at least 96%, at least 97%, at least 98%, at least 99%, or 100% sequence identity).

In some embodiments, a monkeypox antigen (e.g., a T cell antigen for monkeypox) is or includes a H5R polypeptide or fragment thereof. In various embodiments, a H5R polypeptide or fragment thereof has at least 80% sequence identity with a H5R amino acid sequence set forth in Table 2 or otherwise known in the art, or a corresponding portion thereof (e.g., at least 80%, at least 85%, at least 90%, at least 91%, at least 92%, at least 93%, at least 94%, at least 95%, at least 96%, at least 97%, at least 98%, at least 99%, or 100% sequence identity).

In some embodiments, a monkeypox antigen (e.g., a T cell antigen for monkeypox) is or includes an I3L polypeptide or fragment thereof. In various embodiments, an I3L polypeptide or fragment thereof has at least 80% sequence identity with an I3L amino acid sequence set forth in Table 2 or otherwise known in the art, or a corresponding portion thereof (e.g., at least 80%, at least 85%, at least 90%, at least 91%, at least 92%, at least 93%, at least 94%, at least 95%, at least 96%, at least 97%, at least 98%, at least 99%, or 100% sequence identity).

In some embodiments, a monkeypox antigen (e.g., a T cell antigen for monkeypox) is or includes an O2L polypeptide or fragment thereof. In various embodiments, an O2L polypeptide or fragment thereof has at least 80% sequence identity with an O2L amino acid sequence set forth in Table 2 or otherwise known in the art, or a corresponding portion thereof (e.g., at least 80%, at least 85%, at least 90%, at least 91%, at least 92%, at least 93%, at least 94%, at least 95%, at least 96%, at least 97%, at least 98%, at least 99%, or 100% sequence identity).

In some embodiments, a monkeypox antigen (e.g., a T cell antigen for monkeypox) is or includes a Q1L polypeptide or fragment thereof. In various embodiments, a Q1L polypeptide or fragment thereof has at least 80% sequence identity with a Q1L amino acid sequence set forth in Table 2 or otherwise known in the art, or a corresponding portion thereof (e.g., at least 80%, at least 85%, at least 90%, at least 91%, at least 92%, at least 93%, at least 94%, at least 95%, at least 96%, at least 97%, at least 98%, at least 99%, or 100% sequence identity).

In some embodiments, a monkeypox antigen (e.g., a T cell antigen for monkeypox) is or includes a B12R polypeptide or fragment thereof. In various embodiments, a B12R polypeptide or fragment thereof has at least 80% sequence identity with a B12R amino acid sequence set forth in Table 2 or otherwise known in the art, or a corresponding portion thereof (e.g., at least 80%, at least 85%, at least 90%, at least 91%, at least 92%, at least 93%, at least 94%, at least 95%, at least 96%, at least 97%, at least 98%, at least 99%, or 100% sequence identity).

In some embodiments, a monkeypox antigen (e.g., a T cell antigen for monkeypox) is or includes a C17L polypeptide or fragment thereof. In various embodiments, a C17L polypeptide or fragment thereof has at least 80% sequence identity with a C17L amino acid sequence set forth in Table 2 or otherwise known in the art, or a corresponding portion thereof (e.g., at least 80%, at least 85%, at least 90%, at least 91%, at least 92%, at least 93%, at least 94%, at least 95%, at least 96%, at least 97%, at least 98%, at least 99%, or 100% sequence identity).

As disclosed herein, a monkeypox antigen can be or include (i) a polypeptide or fragment thereof of an antigen of Table 1 (e.g., an A29L polypeptide or fragment thereof, A35R polypeptide or fragment thereof, B6R polypeptide or fragment thereof, MIR polypeptide or fragment thereof, E8L polypeptide or fragment thereof, A28L polypeptide or fragment thereof, or H3L polypeptide or fragment thereof) or (ii) a polypeptide or fragment thereof of an antigen of Table 2 (e.g., an A45L polypeptide or fragment thereof, B9R polypeptide or fragment thereof, B16R polypeptide or fragment thereof, C10L polypeptide or fragment thereof, C21L polypeptide or fragment thereof, E7R polypeptide or fragment thereof, F3L polypeptide or fragment thereof, F4L polypeptide or fragment thereof, G6R polypeptide or fragment thereof, H5R polypeptide or fragment thereof, I3L polypeptide or fragment thereof, O2L polypeptide or fragment thereof, Q1L polypeptide or fragment thereof, B12R polypeptide or fragment thereof, or C17L polypeptide or fragment thereof).

In various embodiments, a monkeypox antigen can include an amino acid modification engineered to reduce the number of N-linked glycosylation sites present in the monkeypox antigen sequence (e.g., as compared to a reference). Without wishing to be bound by any particular scientific theory, reduction in the number of N-linked glycosylation sites can be advantageous at least in part because monkeypox antigens expressed during viral infection are not subject to N-linked glycosylation, but monkeypox antigens expressed from a polyribonucleotide of the present disclosure (e.g., when delivered to a host for monkeypox vaccination) may acquire N-linked glycans. This may occur, for example, where a polyribonucleotide-encoded antigen of the present disclosure is operably linked with a signal peptide that targets the antigen to the host secretory system. Such acquisition of N-linked glycans by polyribonucleotide-encoded antigens would potentially introduce structures and/or features (e.g., epitopes) that are not present in virally expressed antigens, and/or eliminate structures and/or features (e.g., eptiopes) that are present in virally expressed antigens, potentially reducing vaccine efficacy.

The present disclosure includes that, in various embodiments, a monkeypox antigen can include an amino acid modification, as compared to a reference sequence that is a corresponding portion of a sequence provided in Table 1 or Table 2, that modifies and/or eliminates an N-linked glycosylation motif that is present in the reference sequence. In particular embodiments, a monkeypox antigen can include an amino acid modification in which an asparagine (N) residue is substituted, e.g., with a glutamine (Q) residue as compared to a reference sequence. In particular embodiments, a monkeypox antigen can include an amino acid modification in which an asparagine (N) residue is substituted with a glutamine (Q) residue as compared to a reference sequence in the context of an N-X-T/S N-linked glycosylation motif of the reference sequence which substitution, absent other changes, would generate a Q-X-T/S motif).

In various embodiments, a monkeypox antigen can include an amino acid modification engineered to reduce the number of unpaired cysteine residues or mispaired cysteine residues present in the monkeypox antigen sequence (e.g., as compared to a reference). Without wishing to be bound by any particular scientific theory, reduction in the number of unpaired cysteine residues can be advantageous at least in part because the presence of unpaired cysteine residues or mispaired cysteine residues carries a risk of causing misfolding and/or aggregation. Antigen misfolding and/or aggregation would potentially introduce structures and/or features (e.g., epitopes) that are not present in virally expressed antigens, and/or eliminate structures and/or features (e.g., eptiopes) that are present in virally expressed antigens, potentially reducing vaccine efficacy. In various embodiments, a monkeypox antigen can include an amino acid modification as compared to a corresponding portion of a sequence provided in Table 1 or Table 2 that substitutes a cysteine residue (e.g., an unpaired or mispaired cysteine residue) with a different residue, such as an alanine residue.

Monkeypox antigens can be encoded by a polyribonucleotide, which polyribonucleotide can be referred to as a monkeypox antigen construct. In various embodiments, a monkeypox antigen construct can be present in a composition for delivery of the monkeypox antigen construct to a subject. In various embodiments, a monkeypox antigen construct can be present in a composition for delivery of one or more monkeypox antigens and/or epitopes to a subject. In various embodiments, a monkeypox antigen construct can be or include a polyribonucleotide that encodes one or more antigens and/or epitopes.

Compositions for delivery of monkeypox antigen constructs and/or monkeypox antigen constructs can, In some embodiments, advantageously include, for example, one or more B cell antigens for monkeypox and one or more T cell antigens (e.g., CD4 and/or CD8 T cell antigens) for monkeypox. Without wishing to be bound by any particular scientific theory, and without suggesting other embodiments are not also advantageous, combination of B cell antigens and T cell antigens can be advantageous in promoting immune system defenses against monkeypox at multiple lifecycle points include without limitation prior to cellular entry and after cellular entry.

The present disclosure includes the recognition that for certain conditions, such as monkeypox, antibodies that target and/or bind monkeypox antigens (e.g., neutralizing antibodies targeted and/or bind monkeypox antigens) can be useful and/or sufficient for treatment of the condition. Accordingly, in various embodiments, the present disclosure provides monkeypox antigen constructs and compositions (e.g., pharmaceutical compositions, e.g., immunogenic compositions, e.g., vaccines) that comprise and/or deliver monkeypox B cell antigens and/or antigen constructs that induce neutralizing antibodies. In some embodiments, the present disclosure provides constructs and compositions that induce robust B cell responses. In some embodiments, a B cell response includes the production of a diverse, specific repertoire of antibodies. In some embodiments, the present disclosure provides monkeypox antigen constructs and compositions (e.g., pharmaceutical compositions, e.g., immunogenic compositions, e.g., vaccines) that comprise and/or deliver antigen constructs that induce both neutralizing antibodies and T cells (e.g., CD4 and/or CD8 T cells). Such neutralizing antibodies and/or T cells (e.g., CD4 and/or CD8 T cells) can target, for example, one or more monkeypox surface proteins. In some embodiments, the present disclosure provides constructs and compositions that induce particularly strong neutralizing antibody responses and/or particularly diverse T cell responses (e.g., targeting multiple T cell epitopes). In some embodiments, the present disclosure provides constructs and compositions that induce T cell and B cell responses to monkeypox antigens and/or epitopes.

The present disclosure provides the recognition, for example, that constructs and compositions comprising polyribonucleotide molecules as described herein (e.g., encoding for one or more monkeypox antigens and/or epitopes) may result in a higher degree of antigen presentation to various immune system components and/or pathways. In some embodiments, administration of such constructs or compositions may induce B cell and/or T cell responses. The present disclosure provides the insight that, e.g., in some embodiments in which B cell and T cell responses are induced in a subject, the subject may have a more sustained, long-term immune response. Such an immune response can be beneficial, e.g., for preventing monkeypox reactivation with a single administration, which may increase vaccination rates and subject compliance as compared with presently available vaccines that require dosing every few years. In some embodiments, constructs and compositions comprising polyribonucleotides as described herein (e.g., encoding for one or more monkeypox antigens and/or epitopes) can provide more diverse protection (e.g., protection against monkeypox variants) because, without wishing to be bound to any particular theory, the constructs and compositions can induce multiple immune system responses.

The present disclosure also provides the recognition that, by administering constructs and compositions that encode monkeypox antigens and/or epitopes, the constructs and compositions described herein avoid administering monkeypox virions, which may infect the subject, go into latency, and/or reactivate to cause a flare-up.

The present disclosure provides a variety of insights and technologies related to monkeypox antigen constructs and vaccine (e.g., a polyribonucleotide vaccine) compositions. In some embodiments, the present disclosure provides particular pharmaceutical composition (e.g., immunogenic composition, e.g., vaccine) formats including, for example, polyribonucleotide pharmaceutical compositions (e.g., immunogenic compositions, e.g., vaccines) comprising particular elements and/or sequences useful for vaccination.

As described herein, in numerous embodiments, provided compositions (e.g., pharmaceutical compositions, immunogenic compositions and/or vaccines) include a polyribonucleotide encoding one or more monkeypox antigens or fragments or epitopes thereof. In some embodiments such a polyribonucleotide is a modified polyribonucleotide in that it includes positions at which uridine residues are substituted with uridine analog(s) such as pseudouridine and/or at which pseudouridine is present. Alternatively or additionally, in some embodiments, a polyribonucleotide includes particular elements (e.g., cap, 5′UTR, 3′UTR, polyA tail, etc.) and/or characteristics (e.g., codon optimization) identified, selected, characterized, and/or demonstrated to achieve and/or increase translatability (e.g., in vitro) and/or expression (e.g., in a subject to whom it has been administered) of encoded protein(s).

In some embodiments, a polyribonucleotide includes particular elements and/or characteristics identified, selected, characterized, and/or demonstrated to achieve significant and/or increased polyribonucleotide stability and/or efficient manufacturing, particularly at large scale (e.g., 0.1-10 g, 10-500 g, 500 g-1 kg, 750 g-1.5 kg; those skilled in the art will appreciate that different products may be manufactured at different scales, e.g., depending on patient population size). In some embodiments, such polyribonucleotide manufacturing scale may be within a range of about 0.01 g/hr polyribonucleotide to about 1 g/hr polyribonucleotide, about 1 g/hr polyribonucleotide to about 100 g/hr polyribonucleotide, about 1 g polyribonucleotide/hr to about 20 g polyribonucleotide/hr, or about 100 g polyribonucleotide/hr to about 10,000 g polyribonucleotide/hr. In some embodiments, polyribonucleotide manufacturing scale may be tens or hundreds of milligrams to tens or hundreds of grams (or more) of polyribonucleotide per batch. In some embodiments, polyribonucleotide manufacturing scale may allow a batch size within a range of about 0.01 g to about 500 g polyribonucleotide, about 0.01 g to about 10 g polyribonucleotide, about 1 g to about 10 g polyribonucleotide, about 10 g to about 500 g polyribonucleotide, about 10 g to about 300 g polyribonucleotide, about 10 g to about 200 g polyribonucleotide or about 30 g to about 60 g polyribonucleotide.

In many embodiments, provided compositions (e.g., pharmaceutical compositions, e.g., immunogenic compositions, e.g., vaccines) that include polyribonucleotide are prepared, formulated, and/or utilized in particular LNP compositions, e.g., as described herein.

Among other things, the present disclosure provides technologies for rapid development of a pharmaceutical composition (e.g., immunogenic composition, e.g., monkeypox vaccine) for delivering particular monkeypox antigen constructs to a subject.

Additionally, the present disclosure provides, for example, nucleic acid constructs encoding monkeypox antigens or fragments thereof disclosed herein, expressing monkeypox antigens or fragments thereof disclosed herein, and various methods of production and/or use relating thereto, as well as compositions developed therewith and methods relating thereto.

The present disclosure provides technologies for preventing, characterizing, treating, and/or monitoring monkeypox outbreaks and/or infections including, e.g., various nucleic acid constructs and encoded proteins, as well as agents (e.g., antibodies) that bind to such proteins, and compositions that comprise and/or deliver them. In some aspects, provided herein are technologies (e.g., compositions and methods) for augmenting, inducing, promoting, enhancing and/or improving an immune response against monkeypox or a component thereof (e.g., a protein or portion thereof). In some embodiments, technologies provided herein are designed to augment, induce, promote, enhance and/or improve immunological memory against monkeypox or a component thereof (e.g., a protein or portion thereof). In some embodiments, technologies described herein are designed to act as an immunological boost to a primary vaccine, such as a vaccine directed to an epitope and/or epitopes of monkeypox. In some embodiments, compositions of the present disclosure comprise one or more polynucleotide constructs (e.g., one or more string constructs) that encode one or more epitopes from monkeypox. In some embodiments, the present disclosure provides vaccines or other compositions comprising nucleic acids encoding such monkeypox epitopes; those skilled in the art will appreciate from context when reference to a particular polynucleotide (e.g., a DNA or RNA) as “encoding” such epitopes in fact is referencing a coding strand or its complement.

### BRIEF DESCRIPTION OF THE DRAWINGS

FIG. 1 is a phylogenetic tree including poxvirus, chordopoxvirus, and orthopoxvirus gene families.

FIG. 2 is a phylogenetic heat map showing percent identities between poxvirus, chordopoxvirus, and orthopoxvirus gene families.

FIG. 3 is a schematic diagram showing orthopoxvirus structure and lifecycle (ViralZone© 2016, Swiss Institute of Bioinformatice, see, https://viralzone.expasy.org/4400).

FIG. 4A is a micrograph and corresponding structural diagram of a vaccinia virus. FIG. 4B is a schematic diagram of the vaccinia virus genome.

FIG. 5 is a schematic diagram illustrating the poxvirus lifecycle.

FIG. 6 is a schematic diagram showing vaccinia virus antigens.

FIG. 7A is a line graph from Hooper et al, J Virol 2004 (https://doi.org/10.1128/JVI.78.9.4433-4443.2004 “Smallpox DNA Vaccine Protects Nonhuman Primates against Lethal Monkeypox”) showing plaque reduction neutralization test (PRNT) 80% neutralization titers in sera collected from Rhesus macaques vaccinated with various smallpox and challenged with monkeypox. FIG. 7B is a table of the corresponding vaccination history and challenge outcome of the Rhesus macaques described in FIG. 7A.

FIG. 8A is a line graph from Fogg et al. J Virol 2004 (DOI: 10.1128/JVI.78.19.10230-10237.2004; “Protective Immunity to Vaccinia Virus Induced by Vaccination with Multiple Recombinant Outer Membrane Proteins of Intracellular and Extracellular Virions”) showing the percentage of surviving mice immunized with single recombinant proteins or combinations of recombinant proteins and challenged with VV-WR.

FIG. 8B is a line graph of the corresponding weights (percentage of initial weight) of the mice described in FIG. 8A.

FIG. 9A is a line graph from Heraud et al. J. Immunol 2016 (https://doi.org/10.4049/jimmunol.177.4.2552; “Subunit Recombinant Vaccine Protects against Monkeypox”) showing serum antibody titers collected from Rhesus macaques immunized with purified monkeypox virus proteins A27Lo (top panel) and B5Ro (bottom panel); the x-axis shows the immunization time in weeks. FIG. 9B is a line graph from Heraud et al. showing serum antibody titers collected from Rhesus macaques immunized with purified monkeypox virus proteins A33Ro (top panel) and L1Ro (bottom panel); the x-axis shows the immunization time in weeks. FIG. 9C is a graph showing kinetic ELISA data (in mOD per minute) of serum antibody responses in Rhesus Macaques immunized with monkeypox proteins or homologous vaccinia proteins.

FIG. 10A is a graph from Heraud et al. J. Immunol 2016 (https://doi.org/10.4049/jimmunol.177.4.2552; “Subunit Recombinant Vaccine Protects against Monkeypox”) showing regression analysis of the percentage of CD4+ T cell responses and the maximum number of lesions measured as CD4+ T cells producing IL-2, where each number refers to data from animal of groups 1-4. FIG. 10B is a graph from Heraud et al. showing regression analysis of the percentage of CD4+ T cell responses and the maximum number of lesions measured as CD4+ T cells producing TNF-α and IFN-7. FIG. 10C is a regression analysis graph of intracellular mature vaccinia virus neutralizing antibody tiers and time to maximum number of pox lesions. Open circles are animals from the group immunized with DNA. Closed circles are animals from the group immunized with protein plus CpG. Open rectangles are animals from the group immunized with protein plus alum. Closed diamonds are animals from the group immunized with DNA plus proteins.

FIG. 11 is a graph from Gilchuk et al. Cell 2016 (DOI: 10.1016/j.cell.2016.09.049; “Cross-Neutralizing and Protective Human Antibody Specificities to Poxvirus Infections”) showing cross neutralization potency (plotted as percent maximum neutralization (Eₘₐₓ), top panel, and IC₅₀, bottom panel) of vaccinia virus (VACV), cowpox virus (CPXV), and monkeypox virus (MPXV) by individual neutralizing human monoclonal antibodies raised against vaccinia virus.

FIG. 12 is a set of Kaplan-Meier graphs from Gilchuk et al. Cell 2016 (DOI: 10.1016/j.cell.2016.09.049; “Cross-Neutralizing and Protective Human Antibody Specificities to Poxvirus Infections”) showing percent survival of BalbC SCID mice challenged with vaccinia virus then inoculated with monoclonal antibodies targeting distinct antigens or combinations thereof.

FIG. 13A is a set of line graphs from Gilchuk et al. Cell 2016 (DOI: 10.1016/j.cell.2016.09.049; “Cross-Neutralizing and Protective Human Antibody Specificities to Poxvirus Infections”) showing body weights of C57BL/6 mice inoculated with individual monoclonal antibodies prior to challenge with vaccinia virus. FIG. 13B is a set of graphs from Gilchuk et al. showing body weights (left panel) and percent survival (right panel) of C57BL/6 mice inoculated with combinations of monoclonal antibodies prior to challenge with vaccinia virus.

FIG. 14 is a set of graphs from Gilchuk et al. Cell 2016 (DOI: 10.1016/j.cell.2016.09.049; “Cross-Neutralizing and Protective Human Antibody Specificities to Poxvirus Infections”) showing cross neutralization potency (plotted as percent maximum neutralization (Eₘₐₓ) vs IC₅₀ of vaccinia virus (VACV), cowpox virus (CPXV), and monkeypox virus (MPXV) mature virons (MV, left panel) and enveloped virions (EV, right panel) by an individual neutralizing human monoclonal antibody raised against vaccinia virus and combinations of monoclonal antibodies.

FIG. 15 is a schematic diagram from Edghill-Smith et al. Nature Medicine 2005 (https://doi.org/10.1038/nm1261) showing the inoculation schedule of control antibody (RSV), anti-CD20 antibody (Rituxan), anti-CD28 antibody (cM-T807), Dryvax, and monkeypox virus challenge to thee groups of macaques.

FIG. 16 is a table from Edghill-Smith et al. Nature Medicine 2005 (https://doi.org/10.1038/nm1261) showing the vaccinia virus antibody titers, monkeypox virus genome concentrations, and incidence of skin pocks in the macaques inoculated as described in FIG. 15.

FIG. 17A and FIG. 17B are a set of graphs from Heraud et al. J. Immunol 2016 (https://doi.org/10.4049/jimmunol.177.4.2552) showing peptide scans performed by ELISA for L1Ro protein. FIG. 17C is a graph of B cell-predicted epitopes obtained from BcePred software and insets showing amino acid sequences of related orthopoxviruses.

FIG. 18A is a ribbon topology diagram showing the structure of smallpox (SPX) L1R from Su et al. 2005. PNAS. FIG. 18B is a ribbon topology diagram showing the binding interaction between the 7D11 neutralizing antibody and LlR. The arrow indicates the binding site at Asp35.

FIG. 19 is a graph showing prediction of transmembrane helices of antigen M1R using TMHMM version 2.0, located at http://www.cbs.dtu.dk/services/TMHMMI.

FIG. 20A is a schematic diagram from Chang et al. 2013. PLoSPathogens, showing the domain structure of SPX A27L. FIG. 20B is a space-filled ribbon topology diagram showing the structure of SPX A27L.

FIG. 21A is a ribbon topology diagram of the structure of poxvirus A33 from S U et al. J Virol. 2010, https://www.ncbi.nlm.nih.gov/pmc/articles/PMC2820914/(top panel) and a schematic diagram of its orientation in the cell membrane (bottom panel). FIG. 21B is a schematic diagram of the orientation of A35R in the cell membrane (left panel). FIG. 21C is a ribbon toplogy diagram of the structure of the A27D7 “protective antibody” from Matho et al. PLoS Pathog. 2015 (https://doi.org/10.1371/journal.ppat.1005148) and its binding interation with the A33 antigen (right panel).

FIG. 22A and FIG. 22B are a set of graphs from Heraud et al. J. Immunol 2016 (https://doi.org/10.4049/jimmunol.177.4.2552) showing peptide scans performed by ELISA for A33Ro protein. FIG. 22C is a graph of B cell-predicted epitopes obtained from BcePred software and insets showing amino acid sequences of related orthopoxviruses.

FIG. 23A is a schematic diagram of the orientation of the monkeypox (MPX) B6R antigen in the cell membrane created using Protter (Ulrich Omasits and others, Protter: interactive protein feature visualization and integration with experimental proteomic data, Bioinformatics, Volume 30, Issue 6, March 2014, Pages 884-886, https://doi.org/10.1093/bioinformatics/btt6O7). FIG. 23B is a space-filled model of the C3b complement protein with smallpox virus SPICE antigen (CCP1-4) from Forneris et al. EMBO J. 2016 (https://doi.org/10.15252/embj.201593673). FIG. 23C is a cartoon representation of the CCP domains (1-4) of the smallpox virus SPICE antigen from Forneris et al.

FIG. 24A and FIG. 24B are a set of graphs from Heraud et al. J. Immunol 2016 (https://doi.org/10.4049/jimmunol.177.4.2552) showing peptide scans performed by ELISA for B5Ro protein. FIG. 24C is a graph of B cell-predicted epitopes obtained from BcePred software and insets showing amino acid sequences of related orthopoxviruses.

FIG. 25 is a schematic diagram from Aldaz-Carroll et al J Virol. 2005 (https://doi.org/10.1128/JVI.79.10.6260-6271.2005) showing the domain structure of vaccinia B5R antigen and the change (n-fold) over background of monoclonal anti-B5R antibody binding to peptides spanning the extracellular domain of B5R.

FIG. 26 is a graph from Aldaz-Carroll et al. J Virol. 2005 (https://doi.org/10.1128/JVI.79.10.6260-6271.2005) showing extracellular enveloped virus (EEV) plaque reduction by anti-B5R(275t) monoclonal antibodies.

FIG. 27 is the amino acid sequence of B5R from Aldaz-Carroll et al. J Virol. 2005 (https://doi.org/10.1128/JVI.79.10.6260-6271.2005) showing the overlapping peptides (black bars) spanning the ectodomain of B5R corresponding to the peptides described in FIG. 25.

FIG. 28A is a schematic diagram created using Protter showing the orientation of the H3L antigen in the cell membrane. FIG. 28B is a ribbon topology diagram from Singh et al., J Virol. 2016 (https://doi.org/10.1128/JVI.02933-15) showing the structure of the H3 antigen.

FIG. 29 is a multiple sequence alignment showing the conservation of the A45L antigen between residues 57-149 across orthopoxvirus family viruses.

FIG. 30 is a multiple sequence alignment showing the conservation of the Q1L antigen between residues 210-346 across orthopoxvirus family viruses.

FIG. 31 is a multiple sequence alignment showing the conservation of the Q1L antigen between residues 546-658 across orthopoxvirus family viruses.

FIG. 32 is a multiple sequence alignment showing the conservation of the B12R antigen between residues 148-244 across orthopoxvirus family viruses.

FIG. 33 is a multiple sequence alignment showing the conservation of the C17L antigen between residues 18-76 across orthopoxvirus family viruses.

FIG. 34 is a multiple sequence alignment showing the conservation of the C17L antigen between residues 185-281 across orthopoxvirus family viruses.

FIG. 35 is a multiple sequence alignment showing the conservation of the I3L antigen between residues 126-199 across orthopoxvirus family viruses.

FIG. 36 is a set of schematic diagrams showing T cell string constructs.

FIG. 37 is a heat map of a subset of the complete monkeypox and caccinia transcriptome from Rubins et al. PLosOne 2008 (https://doi.org/10.1371/journal.pone.0002628) showing early gene expression during poxvirus infection. MPX=Monkeypox Zaire; VAC WR=Vaccinia Western Reserve; E=Early; L=Late.

FIG. 38 is a heat map from Rubins et al. PLosOne 2008 (https://doi.org/10.1371/journal.pone.0002628) showing expression levels of poxvirus gene transcripts whose products are known or predicted to modulate interferon signaling. MPX=Monkeypox Zaire; VAC WR=Vaccinia Western Reserve; E=Early; L=Late.

FIG. 39A is a graph from Assarsson et al. PNAS 2008 (https://doi.org/10.1073/pnas.0711573105) showing the kinetics of orthopoxvirus gene expression for groups of genes associated with a specific function, with the fraction (%) of each group belonging to each kinetic class (Immediate-Early, Early, Early/Late, or Late). FIG. 39B is a line graph from Assarsson et al. showing mean orthopoxvirus open reading frame expression at each time point for genest within each kinetic class (as described in FIG. 39A).

FIG. 40A is a set of line graphs from Croft et al. Mol Cell Proteomics 2015 (https://doi.org/10.1074/mcp.M114.047373) showing cluster analysis of vaccinia protein gene expression over a first timecourse. FIG. 40B is a set of line graphs from Croft et al. showing cluster analysis of vaccinia protein gene expression over a second timecourse. Bar graphs show the relative percentage of proteins falling into each temporal classification within each cluster.

FIG. 41 is a table showing epitope densities for monkeypox virus (MPXV) antigens and the identification of exemplary T cell epitopes.

FIG. 42A is a schematic diagram of the full-length M1R antigen and the corresponding amino acid sequence. FIG. 42B is a schematic diagram of the full-length M1R antigen with an N-terminal HSV-1 gD signal peptide/secretory domain, and the corresponding amino acid sequence.

FIG. 43A is a schematic diagram of the full-length A29L antigen and the corresponding amino acid sequence. FIG. 43B is a schematic diagram of the full-length A29L antigen with an N-terminal HSV-1 gD signal peptide/secretory domain, and the corresponding amino acid sequence. FIG. 43C is a schematic diagram of the full-length A29L antigen having C71A and C72A substitutions with an N-terminal HSV-1 gD signal peptide/secretory domain, and the corresponding amino acid sequence.

FIG. 44A is a schematic diagram of the full-length A35R antigen and the corresponding amino acid sequence. FIG. 44B is a schematic diagram of a polypeptide construct comprising an N-terminal HSV-1 gD signal peptide/secretory domain and two extracellular domains (ECDs) of the A35R antigen connected by a linker, and the corresponding amino acid sequence.

FIG. 45A is a schematic diagram of the full-length B6R antigen and the corresponding amino acid sequence. FIG. 45B is a schematic diagram of the full-length B6R antigen having a C140A substitution, and the corresponding amino acid sequence.

FIG. 46A is a schematic diagram of the full-length H3L antigen and the corresponding amino acid sequence. FIG. 46B is a schematic diagram of the full-length H3L antigen with an N-terminal HSV-1 gD signal peptide/secretory domain, and the corresponding amino acid sequence. FIG. 46C is a schematic diagram of the full-length H3L antigen having C86A and C90A substitutions with an N-terminal HSV-1 gD signal peptide/secretory domain, and the corresponding amino acid sequence.

FIG. 47A is a schematic diagram of the full-length E8L antigen and the corresponding amino acid sequence. FIG. 47B is a schematic diagram of the full-length E8L antigen with an N-terminal HSV-1 gD signal peptide/secretory domain, and the corresponding amino acid sequence.

FIG. 48A is a schematic diagram of the full-length A28L antigen and the corresponding amino acid sequence. FIG. 48B is a schematic diagram of the full-length A28L antigen with an N-terminal HSV-1 gD signal peptide/secretory domain, and the corresponding amino acid sequence.

FIG. 49 is a schematic diagram of a bicistronic construct encoding A29L and A28L antigens.

FIG. 50 is a schematic diagram showing a set of T cell string constructs.

FIG. 51 is a set of graphs showing epitope mapping analyses of the A45L antigen.

FIG. 52 is a set of graphs showing epitope mapping analyses of the Q1L antigen.

FIG. 53 is a set of graphs showing epitope mapping analyses of the B12R antigen.

FIG. 54 is a set of graphs showing epitope mapping analyses of the C17L antigen.

FIG. 55 is a set of graphs showing epitope mapping analyses of the I3L antigen.

FIG. 56 is a table showing characteristics of certain antigens of the present disclosure.

FIG. 57 is a schematic diagram showing an optional immunization protocol for mouse studies.

FIG. 58A-58E are graphs showing the transfection efficiency of exemplary CSP antigen constructs encoding E8 (FIG. 58A), M1 (FIG. 58B), A35 (FIG. 58C), B6 (FIG. 58D), and H3 (FIG. 58E), in mammalian cells (e.g., HEK293 cells) at varying RNA concentrations.

FIG. 59 is a graph depicting in vitro expression of exemplary CSP antigen constructs encoding A35, B6, M1, E8, and H3 antigens in mammalian cells (e.g., HEK293 cells). Heights of bars indicate the mean fluorescence intensity; data are the mean of 2 to 3 technical replicates. NT indicates non-transfected control for each primary antibody stain. WT refers to a wild-type antigen sequence, B6 C140A refers to a B6 antigen with a C140A substitution, +SP indicates the antigen includes a secretion signal (e.g., M1 WT+SP refers to an M1 antigen having a secretion signal, e.g., SS+MIR as described in Table 9, H3 CCAA+SP refers to an H3 antigen having a secretion signal and C86A and C90A substitutions.

FIG. 60 is a schematic diagram of an exemplary immunization protocol for mouse immunization and germinal center induction studies.

FIG. 61A and FIG. 61B are line graphs showing antibody responses in mice immunized with an A35 antigen construct. FIG. 61A is a line graph of serum anti-A35 IgG concentrations in mice immunized with a wild-type A35 polyribonucleotide construct. FIG. 61B is a line graph showing serum anti-A35 IgG concentrations in mice immunized with a A35 secreted dimeric polyribonucleotide construct. Vertical axis depicts antibody levels in serum detected (ng/mL), horizontal axis indicates days post immunization according to the immunization protocol depicted in FIG. 60.

FIG. 62A and FIG. 62B are graphs showing anti-A35 antibody responses in mice immunized with an A35 antigen construct and combinations including the same. FIG. 62A is a graph showing serum anti-A35 IgG concentrations at day 21 post immunization according to the immunization protocol depicted in FIG. 60. FIG. 62B is a graph showing serum anti-A35 IgG concentrations at day 35 post immunication and boost. Vertical axis depicts antibody levels in serum detected (ng/mL). Combo 1 comprises B6 and M1 antigens; Combo 2 comprises A35, B6, and M1 antigens; Combo 3 comprises A35, B6, M1, and E8 antigens; Combo 4 comprises A35, B6, M1, and H3 antigens; and Combo 5 comprises A35 and B6 antigens.

FIG. 63A and FIG. 63B are line graphs showing serum anti-B6 IgG concentrations in mice immunized with B6 antigen constructs. FIG. 63A is a line graph showing serum anti-B6 IgG concentrations of mice immunized with a wild-type B6 polyribonucleotide construct. FIG. 63B is a line graph depicting serum anti-B6 IgG concentrations of mice immunized with a B6, C140A variant polyribonucleotide construct. Vertical axis depicts antibody levels in serum detected (ng/mL), horizontal axis indicates days post immunization according to the immunization protocol depicted in FIG. 60.

FIG. 64A and FIG. 64B are graphs showing antibody responses in mice immunized with a B6 antigen construct and combinations including the same. FIG. 64A is a graph depicting serum anti-B6 IgG concentrations at day 21 post immunization according to the immunization protocol depicted in FIG. 60. FIG. 64B is a graph depicting serum anti-B6 IgG concentrations at day 35 post immunication and boost. Vertical axis depicts antibody levels in serum detected (ng/mL). Combo 1 comprises B6 and M1 antigens; Combo 2 comprises A35, B6, and M1 antigens; Combo 3 comprises A35, B6, M1, and E8 antigens; Combo 4 comprises A35, B6, M1, and H3 antigens; and Combo 5 comprises A35 and B6 antigens.

FIG. 65 is a line graph showing anti-M1 IgG concentrations in mice immunized with an M1 antigen construct. Vertical axis depicts antibody levels in serum detected (ng/mL), horizontal axis indicates days post immunization according to the immunization protocol depicted in FIG. 60.

FIG. 66A and FIG. 66B are graphs showing antibody responses in mice immunized with an M1 antigen construct and combinations including the same. FIG. 66A is a graph depicting serum anti-M1 IgG concentrations at day 21 post immunization according to the immunization protocol depicted in FIG. 60. FIG. 66B is a graph depicting serum anti-M1 IgG concentrations at day 35 post immunization and boost. Vertical axis depicts antibody levels in serum detected (ng/mL). Combo 1 comprises B6 and M1 antigens; Combo 2 comprises A35, B6, and M1 antigens; Combo 3 comprises A35, B6, M1, and E8 antigens; and Combo 4 comprises A35, B6, M1, and H3 antigens.

FIG. 67 is a line graph showing serum anti-E8 IgG concentrations in mice immunized with an E8 antigen construct. Vertical axis depicts antibody levels in serum detected (ng/mL), horizontal axis indicates days post immunization according to the immunization protocol depicted in FIG. 60.

FIG. 68A and FIG. 68B are graphs showing antibody responses in mice immunized with an E8 antigen construct and a combination including the same. FIG. 68A is a graph depicting serum anti-E8 IgG concentrations at day 21 post immunization according to the immunization protocol depicted in FIG. 60. FIG. 68B is a graph depicting serum anti-E8 IgG concentrations at day 35 post immunication and boost. Vertical axis depicts antibody levels in serum detected (ng/mL). Combo 3 comprises A35, B6, M1, and E8 antigens.

FIG. 69A and FIG. 69B are line graphs showing antibody response in mice immunized with H3 antigen constructs. FIG. 69A is a line graph depicting serum anti-H3 IgG concentrations in mice immunized with a wild-type H3 polyribonucleotide construct.

FIG. 69B is a line graph depicting serum anti-H3 IgG concentrations in mice immunized with a H3, C86A/C90A variant polyribonucleotide construct. Vertical axis depicts antibody levels in serum detected (ng/mL), horizontal axis indicates days post immunization according to the immunization protocol depicted in FIG. 60.

FIG. 70A and FIG. 70B are graphs showing antibody responses in mice immunized with an H3 antigen construct and a combination including the same. FIG. 70A is a graph depicting serum anti-H3 IgG concentrations at day 21 post immunization according to the immunization protocol depicted in FIG. 60. FIG. 70B is a graph depicting serum anti-H3 IgG concentrations at day 35 post immunication and boost. Vertical axis depicts antibody levels in serum detected (ng/mL). Combo 4 comprises A35, B6, M1, and H3 antigens.

FIG. 71 is a line graph showing serum anti-A29 IgG concentrations in mice immunized with an A29 C71A/C72A variant antigen construct. Vertical axis depicts antibody levels in serum detected (ng/mL), horizontal axis indicates days post immunization according to the immunization protocol depicted in FIG. 60.

FIG. 72 is a graph showing the effective concentrations of day 35 sera collected from mice immunized with a construct encoding an A35, B6, M1, A29, E8, H3 antigen, or combination thereof, to achieve 50% neutralization (EC₅₀) of monkeypox virus in an in vitro neutralization assay. Combo 1 comprises B6 and M1 antigens; Combo 2 comprises A35, B6, and M1 antigens; Combo 3 comprises A35, B6, M1, and E8 antigens; and Combo 4 comprises A35, B6, M1, and H3 antigens.

FIG. 73 is a graph showing the effective concentrations of day 35 sera collected from mice immunized with constructs encoding an A35, B6, M1, A29, E8, H3 antigen, or combination thereof, to achieve 50% neutralization (EC₅₀) of monkeypox virus in an in vitro neutralization assay in the presence of complement. Combo 1 comprises B6 and M1 antigens; Combo 2 comprises A35, B6, and M1 antigens; Combo 3 comprises A35, B6, M1, and E8 antigens; and Combo 4 comprises A35, B6, M1, and H3 antigens.

FIG. 74A is a set of flow cytometry dot plots showing cell surface expression of various germinal center induction markers. FIG. 74B is a bar graph showing the percentage of germainal center B cells as a proportion of all B cells in mice immunized with an A35, B6, M1, E8, or H3 antigen construct (percentages are means±SEM). FIG. 74C is a bar graph showing the percentage of antigen-specific B cells as a proportion of all germinal center B Cells in mice immunized with an A35, B6, M1, E8, or H3 antigen construct (percentages are means±SEM).

FIGS. 75A-75H are a set of bar graphs showing flow cytometry analysis of HEK293T cells 18 hours post-transfection with combination of nucleic acid constructs encoding combinations of antigens (Combo 3 or Combo 4) or polyribonucleotides encoding single MPXV antigens (A35, B6, M1, or H3). FIG. 75A and FIG. 75B are bar graphs showing cell viability and antigen expression, respectively, of HEK293T cells transfected with constructs encoding A35 antigen, Combo 3, or Combo 4. FIG. 75C and FIG. 75D are bar graphs showing cell viability and antigen expression, respectively, of HEK293T cells transfected with constructs encoding B6 antigen, Combo 3, or Combo 4. FIG. 75E and FIG. 75F are bar graphs showing cell viability and antigen expression, respectively, of HEK293T cells transfected with constructs encoding M1 antigen, Combo 3, or Combo 4. FIG. 75G and FIG. 75G are bar graphs showing cell viability and antigen expression, respectively, of HEK293T cells transfected with constructs encoding H3 antigen, Combo 3, or Combo 4. Combo 3 comprises A35, B6, M1, and E8 antigens; and Combo 4 comprises A35, B6, M1, and H3 antigens. Cell viability graphs of cells transfected are presented as percent of all cells. Antigen expressions are presented as mean fluorescence intensity (MFI) of total live cells under intracellular (total protein) and surface (surface protein) staining conditions. Data are mean of technical triplicates (±standard deviation). NT=non-transfected, NA=not applicable (i.e., candidate vaccine does not encode the MPXV antigen being measured).

FIGS. 76A-76D are line graphs showing serum levels of MPXV antigen-specific immunoglobulin G (IgG) collected from Balb/C mice immunized with compositions comprising constructs encoding combinations of antigens (Combo 3 or Combo 4) or lipid nanoparticles (LNPs) incorporating polyribonucleotides encoding single MPXV antigens (A35, B6, M1, or H3) on days 0 and 21. Serum samples were collected weekly until day 35 and IgG levels for MPXV antigens A35 (FIG. 76A), B6 (FIG. 76B), M1 (FIG. 76C), and H3 (FIG. 76D) were measured at each timepoint by enzyme-linked immunosorbent assay (ELISA). SEM=standard error of the mean. Combo 3 comprises A35, B6, M1, and E8 antigens; and Combo 4 comprises A35, B6, M1, and H3 antigens

FIG. 77A and FIG. 77B are graphs showing MPXV-neutralizing activities of day 35 serum samples collected from Balb/C mice immunized with compositions comprising constructs encoding combinations of antigens (Combo 3 or Combo 4) or modified polyribonucleotides encoding single MPXV antigens (A35, B6, M1, or H3) on days 0 and 21, as measured by plaque reduction neutralization test (PRNT). FIG. 77A and FIG. 77B show 50% MPXV-neutralizing antibody titers (NT₅₀) measured in the absence or presence of baby rabbit complement, respectively. Dashed lines indicate the limits of detection set at half the lowest test serum dilution and twice the highest dilution tested. Combo 3 comprises A35, B6, M1, and E8 antigens; and Combo 4 comprises A35, B6, M1, and H3 antigens

FIGS. 78A-78E are graphs showing antigen-specific IFNγ+ T cell responses in whole splenocyte samples collected from Balb/C mice 7 days post immunization with compositions comprising constructs encoding combinations of antigens (Combo 3 of Combo 4) or polyribonucleotides encoding single MPXV antigens A35, B6, M1, E8, or H3). FIG. 78A, FIG. 78B, FIG. 78C, FIG. 78D, and FIG. 78E show the number of IFNγ+ T cells (per million splenocytes) responsive to A35, B6, M1, E8, and H3, respectively, as measured by ELISpot assay. Each datapoint represents an individual animal with median bars for each group indicated.

FIG. 79A is a schematic diagram showing an immunization schedule for testing the efficacy of combination polyribonucleotide vaccines in CAST/Ei mice intranasally infected with hMPXV/USA/MA001/2022 monkeypox isolates. FIG. 79B is a graph showing monkeypox virus titers in the lungs of CAST/Ei mice immunized with controls (saline or a LNP incorporating a combination of polyribonucleotides encoding A35 and B6) or compositions comprising constructs encoding combinations (Combo 1, Combo 2, Combo 3, or Combo 4) 3 days post intransal infection with hMPXV/USA/MA001/2022 monkeypox isolates. FIG. 79C is a graph of monkeypox virus titers in the lungs of CAST/Ei mice immunized with controls (saline or a LNP incorporating a combination of polyribonucleotides encoding A35 and B6) or compositions comprising constructs encoding combinations (Combo 1, Combo 2, Combo 3, or Combo 4) 7 days post intransal infection with hMPXV/USA/MA001/2022 monkeypox isolates. Combo 1 comprises B6 and M1 antigens; Combo 2 comprises A35, B6, and M1 antigens; Combo 3 comprises A35, B6, M1, and E8 antigens; and Combo 4 comprises A35, B6, M1, and H3 antigens.

FIG. 80 is a graph showing VACV-neutralizing activities of day 35 serum samples collected from Balb/C mice immunized with compositions comprising constructs encoding combinations of antigens (Combo 1, Combo 2, Combo 3 or Combo 4) or modified polyribonucleotides encoding single MPXV antigens (A35, B6, M1, A29, E8, or H3) on days 0 and 21, as measured by plaque reduction neutralization test (PRNT). FIG. 80 show 50% MPXV-neutralizing antibody titers (NT50). Dashed lines indicate the limits of detection set at half the lowest test serum dilution and twice the highest dilution tested. CCAA+SP refers to an H3 antigen having a secretion signal and C86A and C90A substitutions. WT represents the Wild type version of the antigen, and SP represents a version of the antigen with a non-wild type signal peptide or secretion signal. Combo 1 comprises B6 and M1 antigens; Combo 2 comprises A35, B6, and M1 antigens; Combo 3 comprises A35, B6, M1, and E8 antigens; Combo 4 comprises A35, B6, M1, and H3 antigens; and Combo 5 comprises A35 and B6 antigens.

FIG. 81A-B are a set of line graphs showing body weight and survival of BALB/c mice immunized with compositions comprising constructs encoding combinations of antigens (Combo 1, Combo 2, Combo 3, Combo 4, Combo 5) or saline prior to challenge with vaccinia virus. FIG. 81A is a line graph showing body weights and FIG. 81B shows percent survival of BALB/c mice immunized with compositions comprising constructs encoding combinations of antigens (Combo 1, Combo 2, Combo 3, Combo 4, Combo 5) or saline before challenge with vaccinia virus. Combo 1 comprises B6 and M1 antigens; Combo 2 comprises A35, B6, and M1 antigens; Combo 3 comprises A35, B6, M1, and E8 antigens; Combo 4 comprises A35, B6, M1, and H3 antigens; and Combo 5 comprises A35 and B6 antigens.

### DEFINITIONS

Compounds of this disclosure include those described generally above and are further illustrated by the classes, subclasses, and species disclosed herein. As used herein, the following definitions shall apply unless otherwise indicated. For purposes of this disclosure, the chemical elements are identified in accordance with the Periodic Table of Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

Unless otherwise stated, structures depicted herein are meant to include all stereoisomeric (e.g., enantiomeric or diastereomeric) forms of the structure, as well as all geometric or conformational isomeric forms of the structure. For example, the R and S configurations of each stereocenter are contemplated as part of the disclosure. Therefore, single stereochemical isomers, as well as enantiomeric, diastereomeric, and geometric (or conformational) mixtures of provided compounds are within the scope of the disclosure. For example, in some cases, provided compounds show one or more stereoisomers of a compound, and unless otherwise indicated, represents each stereoisomer alone and/or as a mixture. Unless otherwise stated, all tautomeric forms of provided compounds are within the scope of the disclosure.

Unless otherwise indicated, structures depicted herein are meant to include compounds that differ only in the presence of one or more isotopically enriched atoms. For example, compounds having the present structures including replacement of hydrogen by deuterium or tritium, or replacement of a carbon by 13C- or 14C-enriched carbon are within the scope of this disclosure.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Agent: As used herein, the term “agent,” may refer to a physical entity. In some embodiments, an agent may be characterized by a particular feature and/or effect. For example, as used herein, the term “therapeutic agent” refers to a physical entity has a therapeutic effect and/or elicits a desired biological and/or pharmacological effect. In some embodiments, an agent may be a compound, molecule, or entity of any chemical class including, for example, a small molecule, polypeptide, nucleic acid, saccharide, lipid, metal, or a combination or complex thereof.

Aliphatic: The term “aliphatic” refers to a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point or more than one points of attachment to the rest of the molecule. Unless otherwise specified, aliphatic groups contain 1-12 aliphatic carbon atoms. In some embodiments, aliphatic groups contain 1-6 aliphatic carbon atoms (e.g., C₁₋₆). In some embodiments, aliphatic groups contain 1-5 aliphatic carbon atoms (e.g., C₁₋₅). In other embodiments, aliphatic groups contain 1-4 aliphatic carbon atoms (e.g., C₁₋₄). In still other embodiments, aliphatic groups contain 1-3 aliphatic carbon atoms (e.g., C₁₋₃), and in yet other embodiments, aliphatic groups contain 1-2 aliphatic carbon atoms (e.g., C₁₋₂). Suitable aliphatic groups include, but are not limited to, linear or branched, substituted or unsubstituted alkyl, alkenyl, or alkynyl groups and hybrids thereof. A preferred aliphatic group is C₁₋₆ alkyl.

Alkyl: The term “alkyl,” used alone or as part of a larger moiety, refers to a saturated, optionally substituted straight or branched chain hydrocarbon group having (unless otherwise specified) 1-12, 1-10, 1-8, 1-6, 1-4, 1-3, or 1-2 carbon atoms (e.g., C₁₋₁₂, C₁₋₁₀, C₁₋₈, C₁₋₆, C₁₋₄, C₁₋₃, or C₁₋₂). Exemplary alkyl groups include methyl, ethyl, propyl, butyl, pentyl, hexyl, and heptyl.

Alkylene: The term “alkylene” is refers to a bivalent alkyl group. In some embodiments, “alkylene” is a bivalent straight or branched alkyl group. In some embodiments, an “alkylene chain” is a polymethylene group, i.e., —(CH₂)ₙ—, wherein n is a positive integer, e.g., from 1 to 6, from 1 to 4, from 1 to 3, from 1 to 2, or from 2 to 3. An optionally substituted alkylene chain is a polymethylene group in which one or more methylene hydrogen atoms is optionally replaced with a substituent. Suitable substituents include those described below for a substituted aliphatic group and also include those described in the specification herein. It will be appreciated that two substituents of the alkylene group may be taken together to form a ring system. In some embodiments, two substituents can be taken together to form a 3- to 7-membered ring. The substituents can be on the same or different atoms. The suffix “-ene” or “-enyl” when appended to certain groups herein are intended to refer to a bifunctional moiety of said group. For example, “-ene” or “-enyl”, when appended to “cyclopropyl” becomes “cyclopropylene” or “cyclopropylenyl” and is intended to refer to a bifunctional cyclopropyl group, e.g.,

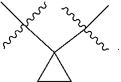 [SMILES: *C1(*)CC1]

Alkenyl: The term “alkenyl”, used alone or as part of a larger moiety, refers to an optionally substituted straight or branched chain or cyclic hydrocarbon group having at least one double bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkenyl groups include ethenyl, propenyl, butenyl, pentenyl, hexenyl, and heptenyl. The term “cycloalkenyl” refers to an optionally substituted non-aromatic monocyclic or multicyclic ring system containing at least one carbon-carbon double bond and having about 3 to about 10 carbon atoms. Exemplary monocyclic cycloalkenyl rings include cyclopentenyl, cyclohexenyl, and cycloheptenyl.

Alkynyl: The term “alkynyl”, used alone or as part of a larger moiety, refers to an optionally substituted straight or branched chain hydrocarbon group having at least one triple bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkynyl groups include ethynyl, propynyl, butynyl, pentynyl, hexynyl, and heptynyl.

Amino acid: In its broadest sense, as used herein, the term “amino acid” refers to a compound and/or substance that can be, is, or has been incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Aryl: The term “aryl” refers to monocyclic and bicyclic ring systems having a total of six to fourteen ring members (e.g., C6-C14), wherein at least one ring in the system is aromatic and wherein each ring in the system contains three to seven ring members. In some embodiments, an “aryl” group contains between six and twelve total ring members (e.g., C6-C12). The term “aryl” may be used interchangeably with the term “aryl ring”. In some embodiments, “aryl” refers to an aromatic ring system which includes, but not limited to, phenyl, biphenyl, naphthyl, anthracyl and the like, which may bear one or more substituents. Unless otherwise specified, “aryl” groups are hydrocarbons. In some embodiments, an “aryl” ring system is an aromatic ring (e.g., phenyl) that is fused to a non-aromatic ring (e.g., cycloalkyl). Examples of aryl rings include that are fused include

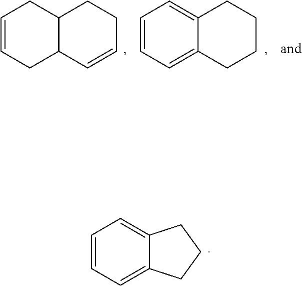

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of, susceptibility to, severity of, stage of, etc the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Co-administration: As used herein, the term “co-administration” refers to use of a composition (e.g., a pharmaceutical composition) described herein and one or more additional therapeutic agents. In some embodiments, one or more additional therapeutic agents comprises at least one polyribonucleotide. The combined use of a composition (e.g., a pharmaceutical composition) described herein and an additional therapeutic agent may be performed concurrently or separately (e.g., sequentially in any order). In some embodiments, a composition (e.g., a pharmaceutical composition) described herein and an additional therapeutic agent may be combined in one pharmaceutically-acceptable excipient, or they may be placed in separate excipient and delivered to a target cell or administered to a subject at different times. Each of these situations is contemplated as falling within the meaning of “co-administration” or “combination,” provided that a composition (e.g., a pharmaceutical composition) described herein and an additional therapeutic agent are delivered or administered sufficiently close in time that there is at least some temporal overlap in biological effect(s) generated by each on a target cell or a subject being treated.

Combination therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, administration of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Corresponding to: As used herein, the term “corresponding to” refers to a relationship between two or more entities. For example, the term “corresponding to” may be used to designate the position/identity of a structural element in a compound or composition relative to another compound or composition (e.g., to an appropriate reference compound or composition). For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure. Those of skill in the art will also appreciate that, in some instances, the term “corresponding to” may be used to describe an event or entity that shares a relevant similarity with another event or entity (e.g., an appropriate reference event or entity). To give but one example, a gene or protein in one organism may be described as “corresponding to” a gene or protein from another organism in order to indicate, in some embodiments, that it plays an analogous role or performs an analogous function and/or that it shows a particular degree of sequence identity or homology, or shares a particular characteristic sequence element.

Cycloaliphatic: As used herein, the term “cycloaliphatic” refers to a monocyclic C₃₋₈ hydrocarbon or a bicyclic C₆₋₁₀ hydrocarbon that is completely saturated or that contains one or more units of unsaturation, but which is not aromatic, that has a single point or more than one points of attachment to the rest of the molecule.

Cycloalkyl: As used herein, the term “cycloalkyl” refers to an optionally substituted saturated ring monocyclic or polycyclic system of about 3 to about 10 ring carbon atoms. Exemplary monocyclic cycloalkyl rings include cyclopropyl, cyclobutyl, cyclopentyl, cyclohexyl, and cycloheptyl.

Derived: In the context of an amino acid sequence (peptide or polypeptide) “derived from” a designated amino acid sequence (peptide or polypeptide), it refers to a structural analogue of a designated amino acid sequence. In some embodiments, an amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof.

Detecting: The term “detecting” is used broadly herein to include appropriate means of determining the presence or absence of an entity of interest or any form of measurement of an entity of interest in a sample. Thus, “detecting” may include determining, measuring, assessing, or assaying the presence or absence, level, amount, and/or location of an entity of interest. Quantitative and qualitative determinations, measurements or assessments are included, including semi-quantitative. Such determinations, measurements or assessments may be relative, for example when an entity of interest is being detected relative to a control reference, or absolute. As such, the term “quantifying” when used in the context of quantifying an entity of interest can refer to absolute or to relative quantification. Absolute quantification may be accomplished by correlating a detected level of an entity of interest to known control standards (e.g., through generation of a standard curve). Alternatively, relative quantification can be accomplished by comparison of detected levels or amounts between two or more different entities of interest to provide a relative quantification of each of the two or more different entities of interest, i.e., relative to each other.

Dosing regimen: Those skilled in the art will appreciate that the term “dosing regimen” (or “therapeutic regimen”) may be used to refer to a set of unit doses (typically more than one) that are administered individually to a subject, typically separated by periods of time. In some embodiments, a given therapeutic agent has a recommended dosing regimen, which may involve one or more doses.

Encode: As used herein, the term “encode” or “encoding” refers to sequence information of a first molecule that guides production of a second molecule having a defined sequence of nucleotides (e.g., a polyribonucleotide) or a defined sequence of amino acids. For example, a DNA molecule can encode an RNA molecule (e.g., by a transcription process that includes a DNA-dependent RNA polymerase enzyme). An RNA molecule can encode a polypeptide (e.g., by a translation process). Thus, a gene, a cDNA, or an RNA molecule encodes a polypeptide if transcription and translation of RNA corresponding to that gene produces the polypeptide in a cell or other biological system. In some embodiments, a coding region of a polyribonucleotide encoding a target antigen refers to a coding strand, the nucleotide sequence of which is identical to the polyribonucleotide sequence of such a target antigen. In some embodiments, a coding region of a polyribonucleotide encoding a target antigen refers to a non-coding strand of such a target antigen, which may be used as a template for transcription of a gene or cDNA.

Engineered: In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature.

Epitope: As used herein, the term “epitope” refers to a moiety that is specifically recognized by an immunoglobulin (e.g., antibody or receptor) binding component. For example, an epitope may be recognized by a T cell, a B cell, or an antibody. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized). Accordingly, in some embodiments, an epitope of an antigen may include a continuous or discontinuous portion of the antigen. In some embodiments, an epitope is or comprises a T cell epitope. In some embodiments, an epitope may have a length of about 5 to about 30 amino acids, or about 10 to about 25 amino acids, or about 5 to about 15 amino acids, or about 5 to 12 amino acids, or about 6 to about 9 amino acids.

Expression: As used herein, the term “expression” of a nucleic acid sequence refers to the generation of a gene product from the nucleic acid sequence. In some embodiments, a gene product can be a transcript, e.g., a polyribonucleotide as provided herein. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, etc); (3) translation of an RNA into a polypeptide or protein; and/or (4) post-translational modification of a polypeptide or protein.

Fragment: As used herein, “fragment” refers a structure that is or includes a discrete portion of a reference agent (sometimes referred to as the “parent” agent). In some embodiments, a fragment lacks one or more moieties found in the reference agent. In some embodiments, a fragment is or includes one or more moieties found in the reference agent. In some embodiments, the reference agent is a polymer such as a polynucleotide or polypeptide. In some embodiments, a fragment of a polymer is or includes at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) of the reference polymer. In some embodiments, a fragment of a polymer is or includes at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the reference polymer. A fragment of a reference polymer is not necessarily identical to a corresponding portion of the reference polymer. For example, a fragment of a reference polymer can be a polymer having a sequence of residues having at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more identity to the reference polymer. A fragment may, or may not, be generated by physical fragmentation of a reference agent. In some instances a fragment is generated by physical fragmentation of a reference agent. In some instances, a fragment is not generated by physical fragmentation of a reference agent and can be instead, for example, produced by de novo synthesis or other means.

Heteroaliphatic: The term “heteroaliphatic” or “heteroaliphatic group,” as used herein, denotes an optionally substituted hydrocarbon moiety having, in addition to carbon atoms, from one to five heteroatoms, that may be straight-chain (i.e., unbranched), branched, or cyclic (“heterocyclic”) and may be completely saturated or may contain one or more units of unsaturation, but which is not aromatic. The term “heteroatom” refers to nitrogen, oxygen, or sulfur, and includes any oxidized form of nitrogen or sulfur, and any quaternized form of a basic nitrogen. The term “nitrogen” also includes a substituted nitrogen. Unless otherwise specified, heteroaliphatic groups contain 1-10 carbon atoms wherein 1-3 carbon atoms are optionally and independently replaced with heteroatoms selected from oxygen, nitrogen, and sulfur. In some embodiments, heteroaliphatic groups contain 1-4 carbon atoms, wherein 1-2 carbon atoms are optionally and independently replaced with heteroatoms selected from oxygen, nitrogen, and sulfur. In yet other embodiments, heteroaliphatic groups contain 1-3 carbon atoms, wherein 1 carbon atom is optionally and independently replaced with a heteroatom selected from oxygen, nitrogen, and sulfur. Suitable heteroaliphatic groups include, but are not limited to, linear or branched, heteroalkyl, heteroalkenyl, and heteroalkynyl groups. For example, a 1- to 10 atom heteroaliphatic group includes the following exemplary groups: —O—CH₃, —CH₂—O—CH₃, —O—CH₂—CH₂—O—CH₂—CH₂—O—CH₃, and the like.

Heteroaryl: The terms “heteroaryl” and “heteroar-”, used alone or as part of a larger moiety, e.g., “heteroaralkyl,” or “heteroaralkoxy,” refer to monocyclic or bicyclic ring groups having 5 to 10 ring atoms (e.g., 5- to 6-membered monocyclic heteroaryl or 9- to 10-membered bicyclic heteroaryl); having 6, 10, or 14 γ-electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, imidazo[1,2-a]pyrimidinyl, imidazo[1,2-a]pyridyl, imidazo[4,5-b]pyridyl, imidazo[4,5-c]pyridyl, pyrrolopyridyl, pyrrolopyrazinyl, thienopyrimidinyl, triazolopyridyl, and benzoisoxazolyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring (i.e., a bicyclic heteroaryl ring having 1 to 3 heteroatoms). Nonlimiting examples include indolyl, isoindolyl, benzothienyl, benzofuranyl, dibenzofuranyl, indazolyl, benzimidazolyl, benzotriazolyl, benzothiazolyl, benzothiadiazolyl, benzoxazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, pyrido[2,3-b]-1,4-oxazin-3(4H)-one, 4H-thieno[3,2-b]pyrrole, and benzoisoxazolyl. The term “heteroaryl” may be used interchangeably with the terms “heteroaryl ring,” “heteroaryl group,” or “heteroaromatic,” any of which terms include rings that are optionally substituted.

Heteroatom: The term “heteroatom” as used herein refers to nitrogen, oxygen, or sulfur, and includes any oxidized form of nitrogen or sulfur, and any quaternized form of a basic nitrogen.

Heterocycle: As used herein, the terms “heterocycle,” “heterocyclyl,” “heterocyclic radical,” and “heterocyclic ring” are used interchangeably and refer to a stable 3- to 8-membered monocyclic, a 6- to 10-membered bicyclic, or a 10- to 16-membered polycyclic heterocyclic moiety that is either saturated or partially unsaturated, and having, in addition to carbon atoms, one or more, such as one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or NR⁺ (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, azetidinyl, oxetanyl, tetrahydrofuranyl, tetrahydrothienyl, pyrrolidinyl, piperidinyl, decahydroquinolinyl, oxazolidinyl, piperazinyl, dioxanyl, dioxolanyl, diazepinyl, oxazepinyl, thiazepinyl, morpholinyl, and thiamorpholinyl. A heterocyclyl group may be mono-, bi-, tri-, or polycyclic, preferably mono-, bi-, or tricyclic, more preferably mono- or bicyclic. A bicyclic heterocyclic ring also includes groups in which the heterocyclic ring is fused to one or more aryl rings. Exemplary bicyclic heterocyclic groups include indolinyl, isoindolinyl, benzodioxolyl, 1,3-dihydroisobenzofuranyl, 2,3-dihydrobenzofuranyl, and tetrahydroquinolinyl. A bicyclic heterocyclic ring can also be a spirocyclic ring system (e.g., 7- to 11-membered spirocyclic fused heterocyclic ring having, in addition to carbon atoms, one or more heteroatoms as defined above (e.g., one, two, three or four heteroatoms)). A bicyclic heterocyclic ring can also be a bridged ring system (e.g., 7- to 11-membered bridged heterocyclic ring having one, two, or three bridging atoms.

Homology: As used herein, the term “homology” or “homolog” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or polypeptide molecules are considered to be “homologous” to one another if their sequences are at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical. In some embodiments, polynucleotide molecules (e.g., DNA molecules and/or RNA molecules) and/or polypeptide molecules are considered to be “homologous” to one another if their sequences are at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution.

Identity: As used herein, the term “identity” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or polypeptide molecules are considered to be “substantially identical” to one another if their sequences are at least 80%, at least 85%, at least 90%, at least 95%, at least 96%, at least 97%, at least 98%, or at least 99% identical. Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In some embodiments, the length of a sequence aligned for comparison purposes is at least 30%, at least 40%, at least 50%, at least 60%, at least 70%, at least 75%, at least 80%, at least 85%, at least 90%, at least 95%, at least 96%, at least 97%, at least 98%, at least 99%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller, 1989, which has been incorporated into the ALIGN program (version 2.0). In some embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Increased, Induced, or Reduced: As used herein, these terms or grammatically comparable comparative terms, indicate values that are relative to a comparable reference measurement. For example, in some embodiments, an assessed value achieved with a provided composition (e.g., a pharmaceutical composition) may be “increased” relative to that obtained with a comparable reference composition. Alternatively or additionally, in some embodiments, an assessed value achieved in a subject may be “increased” relative to that obtained in the same subject under different conditions (e.g., prior to or after an event; or presence or absence of an event such as administration of a composition (e.g., a pharmaceutical composition) as described herein, or in a different, comparable subject (e.g., in a comparable subject that differs from the subject of interest in prior exposure to a condition, e.g., absence of administration of a composition (e.g., a pharmaceutical composition) as described herein). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance. In some embodiments, the term “reduced” or equivalent terms refers to a reduction in the level of an assessed value by at least 5%, at least 10%, at least 20%, at least 50%, at least 75% or higher, as compared to a comparable reference. In some embodiments, the term “reduced” or equivalent terms refers to a complete or essentially complete inhibition, i.e., a reduction to zero or essentially to zero. In some embodiments, the term “increased” or “induced” refers to an increase in the level of an assessed value by at least 10%, at least 20%, at least 30%, at least 40%, at least 50%, at least 80%, at least 100%, at least 200%, at least 500%, or higher, as compared to a comparable reference.

In order: As used herein with reference to a polynucleotide or polyribonucleotide, “in order” refers to the order of features from 5′ to 3′ along the polynucleotide or polyribonucleotide. As used herein with reference to a polypeptide, “in order” refers to the order of features moving from the N-terminal-most of the features to the C-terminal-most of the features along the polypeptide. “In order” does not mean that no additional features can be present among the listed features. For example, if Features A, B, and C of a polynucleotide are described herein as being “in order, Feature A, Feature B, and Feature C,” this description does not exclude, e.g., Feature D being located between Features A and B.

Ionizable: The term “ionizable” refers to a compound or group or atom that is charged at a certain pH. In the context of an ionizable amino lipid, such a lipid or a function group or atom thereof bears a positive charge at a certain pH. In some embodiments, an ionizable amino lipid is positively charged at an acidic pH. In some embodiments, an ionizable amino lipid is predominately neutral at physiological pH values, e.g., in some embodiments about 7.0-7.4, but becomes positively charged at lower pH values. In some embodiments, an ionizable amino lipid may have a pKa within a range of about 5 to about 7.

Isolated: The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Lipid: As used herein, the terms “lipid” and “lipid-like material” are broadly defined as molecules which comprise one or more hydrophobic moieties or groups and optionally also one or more hydrophilic moieties or groups. Molecules comprising hydrophobic moieties and hydrophilic moieties are also typically denoted as amphiphiles.

RNA lipid nanoparticle: As used herein, the term “RNA lipid nanoparticle” refers to a nanoparticle comprising at least one lipid and RNA molecule(s), e.g., one or more polyribonucleotides as provided herein. In some embodiments, an RNA lipid nanoparticle comprises at least one cationic amino lipid. In some embodiments, an RNA lipid nanoparticle comprises at least one cationic amino lipid, at least one helper lipid, and at least one polymer-conjugated lipid (e.g., PEG-conjugated lipid). In various embodiments, RNA lipid nanoparticles as described herein can have an average size (e.g., Z-average) of about 100 nm to 1000 nm, or about 200 nm to 900 nm, or about 200 nm to 800 nm, or about 250 nm to about 700 nm. In some embodiments of the present disclosure, RNA lipid nanoparticles can have a particle size (e.g., Z-average) of about 30 nm to about 200 nm, or about 30 nm to about 150 nm, about 40 nm to about 150 nm, about 50 nm to about 150 nm, about 60 nm to about 130 nm, about 70 nm to about 110 nm, about 70 nm to about 100 nm, about 80 nm to about 100 nm, about 90 nm to about 100 nm, about 70 to about 90 nm, about 80 nm to about 90 nm, or about 70 nm to about 80 nm. In some embodiments, an average size of lipid nanoparticles is determined by measuring the average particle diameter. In some embodiments, RNA lipid nanoparticles may be prepared by mixing lipids with RNA molecules described herein.

Neutralization: As used herein, the term “neutralization” refers to an event in which binding agents such as antibodies bind to a biological active site of a virus such as a receptor binding protein, thereby inhibiting the parasitic infection of cells. In some embodiments, the term “neutralization” refers to an event in which binding agents eliminate or significantly reduce ability of infecting cells.

Nucleic acid/Polynucleotide: As used herein, the term “nucleic acid” refers to a polymer of at least 10 nucleotides or more. In some embodiments, a nucleic acid is or comprises DNA. In some embodiments, a nucleic acid is or comprises RNA. In some embodiments, a nucleic acid is or comprises peptide nucleic acid (PNA). In some embodiments, a nucleic acid is or comprises a single stranded nucleic acid. In some embodiments, a nucleic acid is or comprises a double-stranded nucleic acid. In some embodiments, a nucleic acid comprises both single and double-stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises one or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 6-O-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro), reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or more residues or nucleotides long.

Operably linked: As used herein, “operably linked” refers to the association of at least a first element and a second element such that the component elements are in a relationship permitting them to function in their intended manner. For example, a nucleic acid sequence or amino acid sequence is operably linked with another sequence if it modifies the expression, structure, or activity of the linked sequence, e.g., in an intended manner. In many cases, two nucleic acid sequences are operably linked if they contribute to the expression, structure, or activity of a gene or encoded polypeptide. For example, a nucleic acid regulatory sequence is “operably linked” to a nucleic acid coding sequence if the regulatory sequence and coding sequence are associated in a manner that permits control of expression of the coding sequence by the regulatory sequence. In some embodiments, an “operably linked” regulatory sequence is directly or indirectly covalently associated with a coding sequence (e.g., in a single nucleic acid). In some embodiments, a regulatory sequence controls expression of a coding sequence in trans and inclusion of the regulatory sequence in the same nucleic acid as the coding sequence is not a requirement of operable linkage. In many cases, two amino acid sequences are operably linked if they are expressed as a single polypeptide.

Pharmaceutically effective amount: The term “pharmaceutically effective amount” or “therapeutically effective amount” refers to the amount which achieves a desired reaction or a desired effect alone or together with further doses. In the case of the treatment of a particular disease (e.g., monkeypox), a desired reaction in some embodiments relates to inhibition of the course of the disease (e.g., monkeypox). In some embodiments, such inhibition may comprise slowing down the progress of a disease (e.g., monkeypox) and/or interrupting or reversing the progress of the disease (e.g., monkeypox). In some embodiments, a desired reaction in a treatment of a disease (e.g., monkeypox) may be or comprise delay or prevention of the onset of a disease (e.g., monkeypox) or a condition (e.g., a monkeypox associated condition). An effective amount of a composition (e.g., a pharmaceutical composition) described herein will depend, for example, on disease (e.g., monkeypox) or a condition (e.g., a monkeypox associated condition) to be treated, the severity of such a disease (e.g., Monkeypox) or a condition, individual parameters of the patient, including, e.g., age, physiological condition, size and weight, the duration of treatment, the type of an accompanying therapy (if present), the specific route of administration and similar factors. Accordingly, doses of a composition (e.g., a pharmaceutical composition) described herein may depend on various such parameters. In the case that a reaction in a patient is insufficient with an initial dose, higher doses (or effectively higher doses achieved by a different, more localized route of administration) may be used.

Polypeptide: As used herein, the term “polypeptide” refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications comprise acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 35 or more amino acids; in some embodiments, a conserved region encompasses at least one stretch of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous amino acids. In some embodiments, a relevant polypeptide may comprise or consist of a fragment of a parent polypeptide.

Prevent: As used herein, the terms “prevent” or “prevention” when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Ribonucleic acid (RNA) or Polyribonucleotide: As used herein, the terms “ribonucleic acid,” “RNA,” or “polyribonucleotide” refers to a polymer of ribonucleotides. In some embodiments, an RNA is single stranded. In some embodiments, an RNA is double stranded. In some embodiments, an RNA comprises both single and double stranded portions. In some embodiments, an RNA can comprise a backbone structure as described in the definition of “Nucleic acid/Polynucleotide” above. An RNA can be a regulatory RNA (e.g., siRNA, microRNA, etc.), or a messenger RNA (mRNA). In some embodiments, an RNA is an mRNA. In some embodiments, where an RNA is a mRNA, a RNA typically comprises at its 3′ end a poly(A) region. In some embodiments, where an RNA is a mRNA, an RNA typically comprises at its 5′ end an art-recognized cap structure, e.g., for recognizing and attachment of a mRNA to a ribosome to initiate translation. In some embodiments, a RNA is a synthetic RNA. Synthetic RNAs include RNAs that are synthesized in vitro (e.g., by enzymatic synthesis methods and/or by chemical synthesis methods).

Ribonucleotide: As used herein, the term “ribonucleotide” encompasses unmodified ribonucleotides and modified ribonucleotides. For example, unmodified ribonucleotides include the purine bases adenine (A) and guanine (G), and the pyrimidine bases cytosine (C) and uracil (U). Modified ribonucleotides may include one or more modifications including, but not limited to, for example, (a) end modifications, e.g., 5′ end modifications (e.g., phosphorylation, dephosphorylation, conjugation, inverted linkages, etc.), 3′ end modifications (e.g., conjugation, inverted linkages, etc.), (b) base modifications, e.g., replacement with modified bases, stabilizing bases, destabilizing bases, bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages. The term “ribonucleotide” also encompasses ribonucleotide triphosphates including modified and non-modified ribonucleotide triphosphates.

Risk: As will be understood from context, “risk” of a disease, disorder, and/or condition refers to a likelihood that a particular individual will develop the disease, disorder, and/or condition. In some embodiments, risk is expressed as a percentage. In some embodiments, risk is expressed as a risk relative to a risk associated with a reference sample or group of reference samples. In some embodiments, a reference sample or group of reference samples have a known risk of a disease, disorder, condition and/or event. In some embodiments, a reference sample or group of reference samples are from individuals comparable to a particular individual. In some embodiments, risk may reflect one or more genetic attributes, e.g., which may predispose an individual toward development (or not) of a particular disease, disorder and/or condition. In some embodiments, risk may reflect one or more epigenetic events or attributes and/or one or more lifestyle or environmental events or attributes.

Selective or specific: The terms “selective” or “specific,” when used herein in reference to an agent having an activity, is understood by those skilled in the art to mean that the agent discriminates between potential target entities, states, or cells. For example, in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of a target-binding moiety for one or more other potential target entities (e.g., competitors). In some embodiments, specificity is evaluated relative to that of a reference specific binding moiety. In some embodiments, specificity is evaluated relative to that of a reference non-specific binding moiety.

Substituted or optionally substituted: As described herein, compounds of the invention may contain “optionally substituted” moieties. In general, the term “substituted,” whether preceded by the term “optionally” or not, means that one or more hydrogens of the designated moiety are replaced with a suitable substituent. “Substituted” applies to one or more hydrogens that are either explicit or implicit from the structure (e.g.

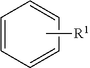

refers to at least

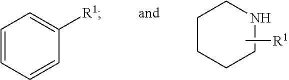

refers to at least

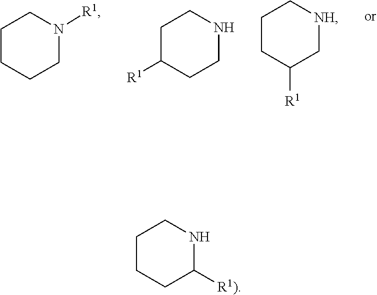

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in some embodiments, their recovery, purification, and use for one or more of the purposes provided herein. Groups described as being “substituted” preferably have between 1 and 4 substituents, more preferably 1 or 2 substituents. Groups described as being “optionally substituted” may be unsubstituted or be “substituted” as described above.

Suitable monovalent substituents on a substitutable carbon atom of an “optionally substituted” group are independently halogen; —(CH₂)₀₋₄Rº; —(CH₂)₀₋₄ORº; —O(CH₂)₀₋₄Rº, —O—(CH₂)₀₋₄C(O)ORº; —(CH₂)₀₋₄CH(ORº)₂; —(CH₂)₀₋₄SRº; —(CH₂)₀₋₄Ph, which may be substituted with Rº; —(CH₂)₀₋₄O(CH₂)₀₋₁Ph which may be substituted with Rº; —CH═CHPh, which may be substituted with Rº; —(CH₂)₀₋₄O(CH₂)₀₋₁-pyridyl which may be substituted with Rº; —NO₂; —CN; —N₃; —(CH₂)₀₋₄N(Rº)₂; —(CH₂)₀₋₄N(Rº)C(O)Rº; —N(Rº)C(S)Rº; —(CH₂)₀₋₄N(Rº)C(O)NRº₂; —N(Rº)C(S)NRº₂; —(CH₂)₀₋₄N(Rº)C(O)ORº; —N(Rº)N(Rº)C(O)Rº; —N(Rº)N(Rº)C(O)NRº₂; —N(Rº)N(Rº)C(O)ORº; —(CH₂)₀₋₄C(O)Rº; C(S)Rº; —(CH₂)₀₋₄C(O)ORº; —(CH₂)₀₋₄C(O)SRº; —(CH₂)₀₋₄C(O)OSiRº₃; —(CH₂)₀₋₄OC(O)Rº; —OC(O)(CH₂)₀₋₄SRº; —(CH₂)₀₋₄SC(O)Rº; —(CH₂)₀₋₄C(O)NRº₂; —C(S)NRº₂; —C(S)SRº; —SC(S)SRº, —(CH₂)₀₋₄OC(O)NRº₂; —C(O)N(ORº)Rº; —C(O)C(O)Rº; —C(O)CH₂C(O)Rº; —C(NORº)Rº; —(CH₂)₀₋₄SSRº; —(CH₂)₀₋₄S(O)₂Rº; —(CH₂)₀₋₄S(O)₂ORº; —(CH₂)₀₋₄OS(O)₂Rº; —S(O)₂NRº₂; —(CH₂)₀₋₄S(O)Rº; —N(Rº)S(O)₂NRº₂; —N(Rº)S(O)₂Rº; —N(ORº)Rº; —C(NH)NRº₂; —P(O)₂Rº; —P(O)Rº₂; —OP(O)Rº₂; —OP(O)(ORº)₂; SiRº₃; —(C₁₋₄ straight or branched alkylene)O—N(Rº)₂; or —(C₁₋₄ straight or branched alkylene)C(O)O—N(Rº)₂, wherein each Rº may be substituted as defined below and is independently hydrogen, C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring having 0-4 heteroatoms independently selected from nitrogen, oxygen, or sulfur, or, notwithstanding the definition above, two independent occurrences of Rº, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on Rº (or the ring formed by taking two independent occurrences of Rº together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂, —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur. Suitable divalent substituents on a saturated carbon atom of Rº include ═O and ═S.

Suitable divalent substituents on a saturated carbon atom of an “optionally substituted” group include the following: ═O (“oxo”), ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5- to 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R is independently hydrogen, C₁₋₆ aliphatic which may be substituted as defined below, unsubstituted —OPh, or an unsubstituted 3- to 6-membered saturated, partially unsaturated, or aryl ring having 0-4 heteroatoms independently selected from nitrogen, oxygen, or sulfur, or, notwithstanding the definition above, two independent occurrences of R, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on the aliphatic group of R are independently halogen, —R^{●}, -(haloR^{●}), —OH, —OR^{●}, —O(haloR^{●}), —CN, —C(O)OH, —C(O)OR^{●}, —NH₂, —NHR, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Subject: As used herein, the term “subject” refers to an organism to be administered with a composition described herein, e.g., for experimental, diagnostic, prophylactic, and/or therapeutic purposes. Typical subjects include animals (e.g., mammals such as mice, rats, rabbits, non-human primates, domestic pets, etc.) and humans. In some embodiments, a subject is a human subject. In some embodiments, a subject is suffering from a disease, disorder, or condition (e.g., orthopox, e.g., monkeypox, variola, or vaccinia). In some embodiments, a subject is susceptible to a disease, disorder, or condition (e.g., orthopox, e.g., monkeypox, variola, or vaccinia). In some embodiments, a subject displays one or more symptoms or characteristics of a disease, disorder, or condition (e.g., orthopox, e.g., monkeypox, variola, or vaccinia). In some embodiments, a subject displays one or more non-specific symptoms of a disease, disorder, or condition (e.g., orthopox, e.g., monkeypox, variola, or vaccinia). In some embodiments, a subject does not display any symptom or characteristic of a disease, disorder, or condition (e.g., orthopox, e.g., monkeypox, variola, or vaccinia). In some embodiments, a subject is someone with one or more features characteristic of susceptibility to or risk of a disease, disorder, or condition (e.g., orthopox, e.g., monkeypox, variola, or vaccinia). In some embodiments, a subject is a patient. In some embodiments, a subject is an individual to whom diagnosis and/or therapy is and/or has been administered.

Suffering from: An individual who is “suffering from” a disease, disorder, and/or condition (e.g., orthopox, e.g., monkeypox, variola, or vaccinia) has been diagnosed with and/or displays one or more symptoms of a disease, disorder, and/or condition.

Susceptible to: An individual who is “susceptible to” a disease, disorder, and/or condition (e.g., orthopox, e.g., monkeypox, variola, or vaccinia) is one who has a higher risk of developing the disease, disorder, and/or condition (e.g., orthopox, e.g., monkeypox, variola, or vaccinia) than does a member of the general public. In some embodiments, an individual who is susceptible to a disease, disorder and/or condition (e.g., orthopox, e.g., monkeypox, variola, or vaccinia) may not have been diagnosed with the disease, disorder, and/or condition (e.g., orthopox, e.g., monkeypox, variola, or vaccinia). In some embodiments, an individual who is susceptible to a disease, disorder, and/or condition (e.g., orthopox, e.g., monkeypox, variola, or vaccinia) may exhibit symptoms of the disease, disorder, and/or condition (e.g., orthopox, e.g., monkeypox, variola, or vaccinia). In some embodiments, an individual who is susceptible to a disease, disorder, and/or condition (e.g., orthopox, e.g., monkeypox, variola, or vaccinia) may not exhibit symptoms of the disease, disorder, and/or condition (e.g., orthopox, e.g., monkeypox, variola, or vaccinia). In some embodiments, an individual who is susceptible to a disease, disorder, and/or condition (e.g., orthopox, e.g., monkeypox, variola, or vaccinia) will develop the disease, disorder, and/or condition (e.g., orthopox, e.g., monkeypox, variola, or vaccinia). In some embodiments, an individual who is susceptible to a disease, disorder, and/or condition (e.g., orthopox, e.g., monkeypox, variola, or vaccinia) will not develop the disease, disorder, and/or condition (e.g., orthopox, e.g., monkeypox, variola, or vaccinia).

Therapy: The term “therapy” refers to an administration or delivery of an agent or intervention that has a therapeutic effect and/or elicits a desired biological and/or pharmacological effect (e.g., has been demonstrated to be statistically likely to have such effect when administered to a relevant population). In some embodiments, a therapeutic agent or therapy is any substance that can be used to alleviate, ameliorate, relieve, inhibit, prevent, delay onset of, reduce severity of, and/or reduce incidence of one or more symptoms or features of a disease, disorder, and/or condition (e.g., orthopox, e.g., monkeypox, variola, or vaccinia). In some embodiments, a therapeutic agent or therapy is a medical intervention (e.g., surgery, radiation, phototherapy) that can be performed to alleviate, relieve, inhibit, present, delay onset of, reduce severity of, and/or reduce incidence of one or more symptoms or features of a disease, disorder, and/or condition.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition (e.g., orthopox, e.g., monkeypox, variola, or vaccinia). Treatment may be administered to a subject who does not exhibit signs of a disease, disorder, and/or condition (e.g., orthopox, e.g., monkeypox, variola, or vaccinia). In some embodiments, treatment may be administered to a subject who exhibits only early signs of the disease, disorder, and/or condition (e.g., orthopox, e.g., monkeypox, variola, or vaccinia), for example for the purpose of decreasing the risk of developing pathology associated with the disease, disorder, and/or condition. In some embodiments, treatment may be administered to a subject at a later-stage of disease, disorder, and/or condition (e.g., orthopox, e.g., monkeypox, variola, or vaccinia).

### DETAILED DESCRIPTION OF CERTAIN EMBODIMENTS

The present disclosure provides pharmaceutical compositions (e.g., immunogenic compositions, e.g., vaccines) for delivering particular monkeypox antigen constructs to a subject (e.g., a patient) and related technologies (e.g., methods). In particular, the present disclosure provides monkeypox vaccine compositions and related technologies (e.g., methods).

### I. Monkeypox and Other Orthopoxviruses

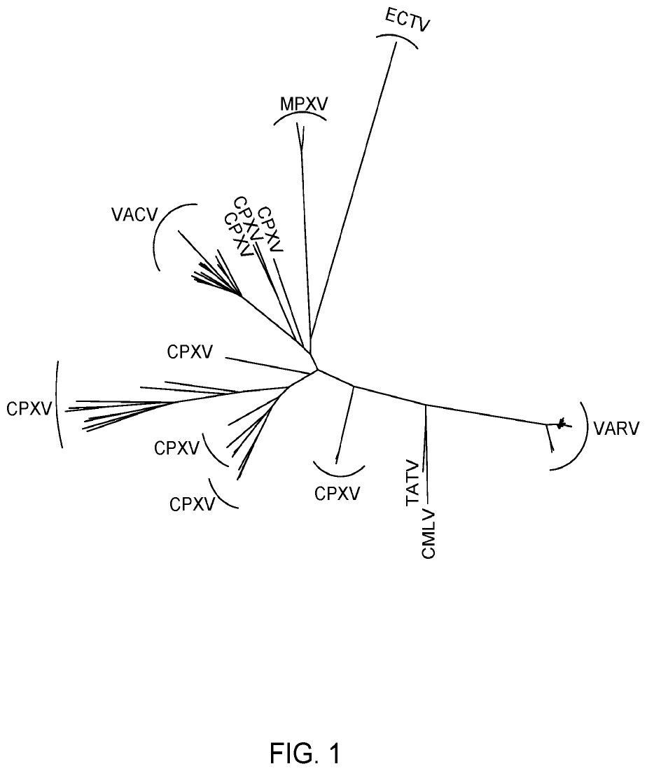

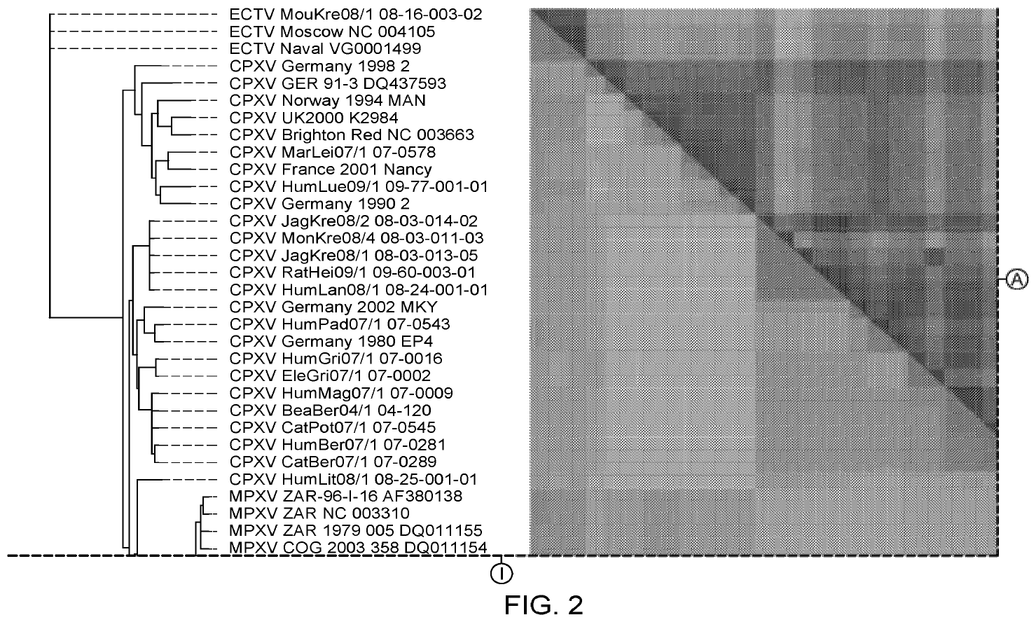

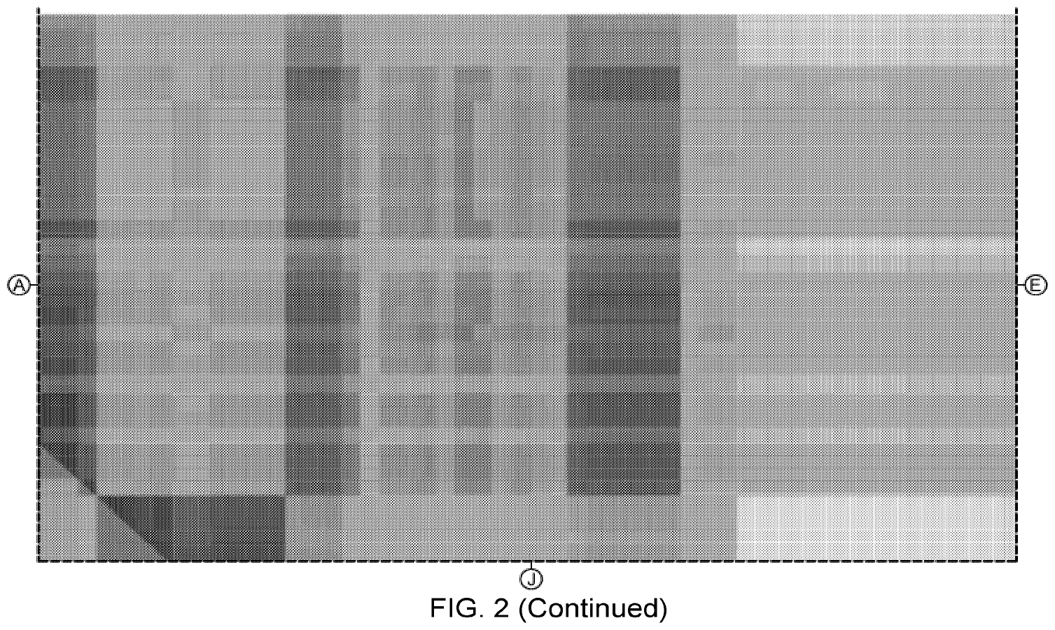

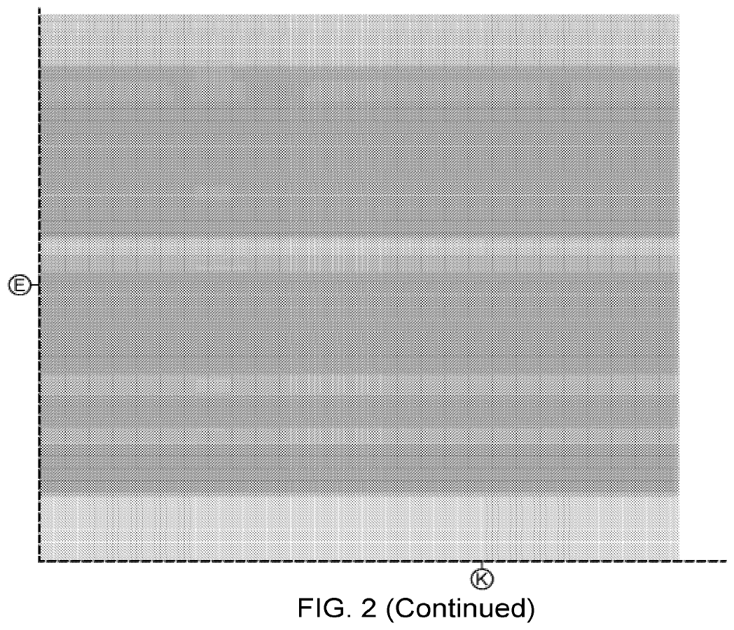

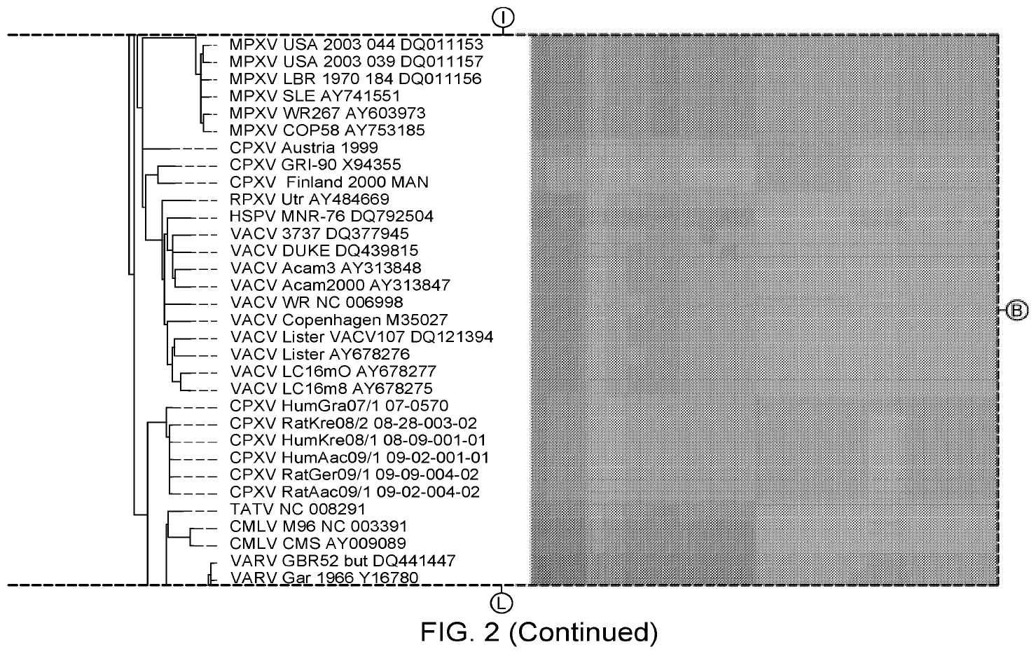

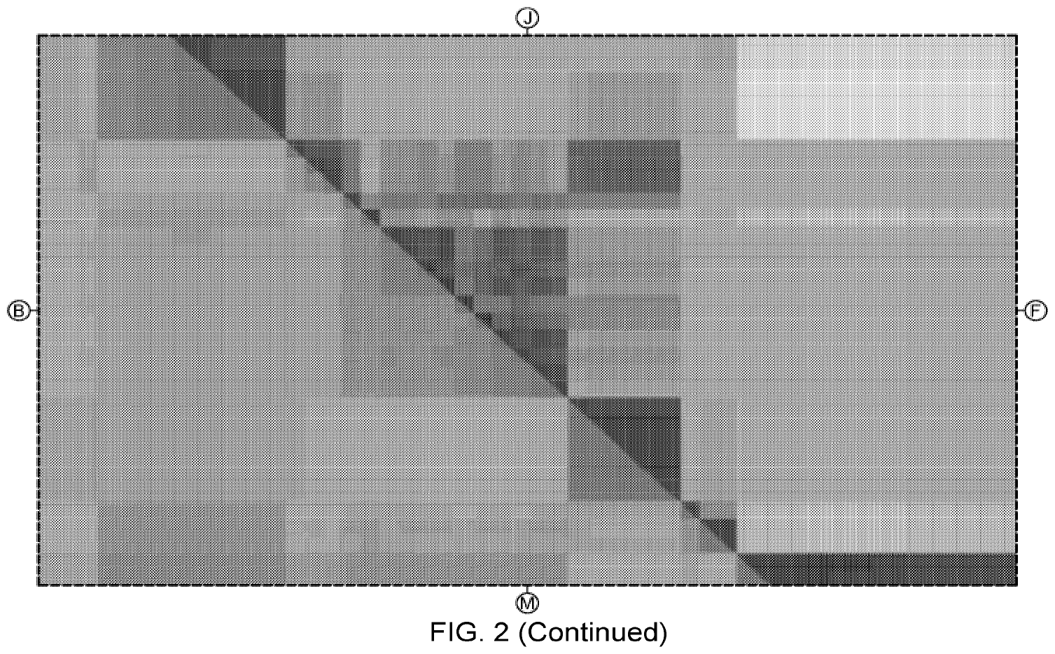

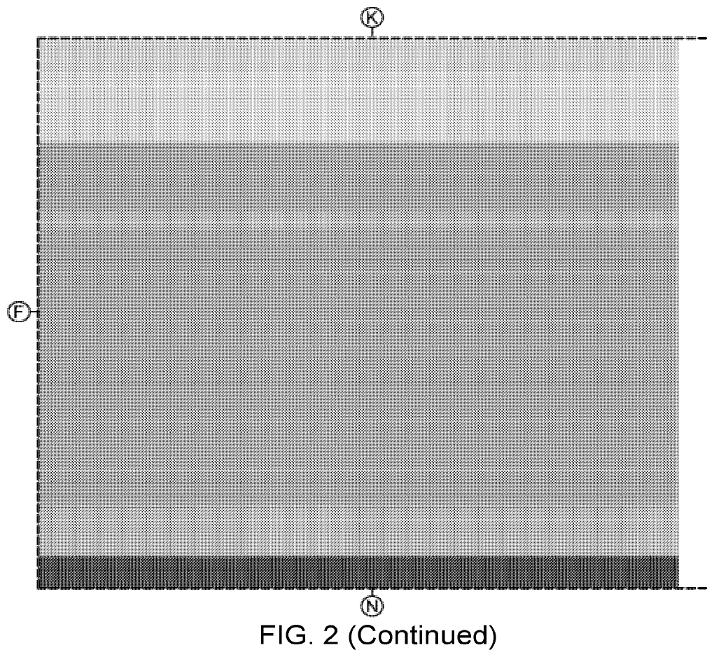

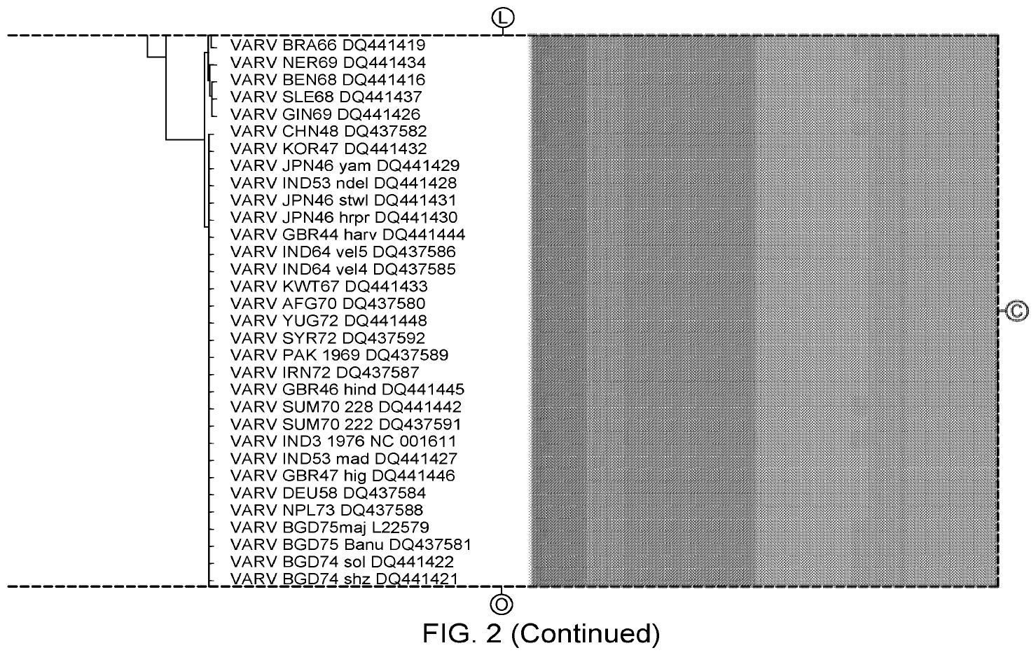

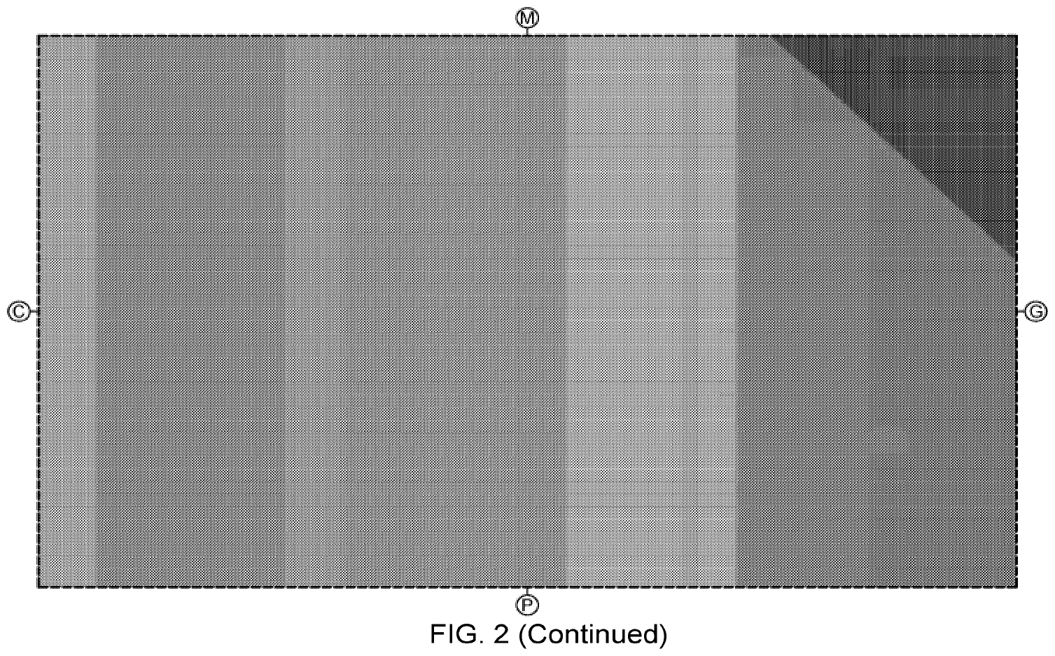

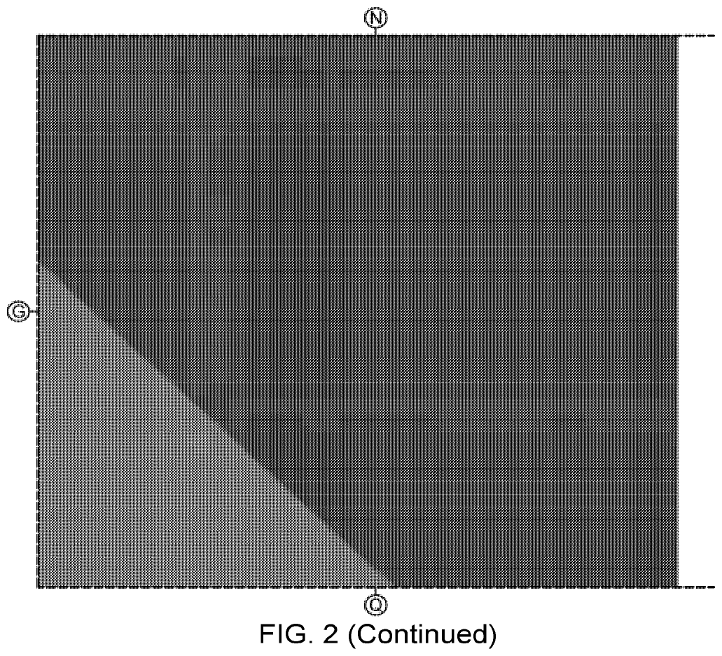

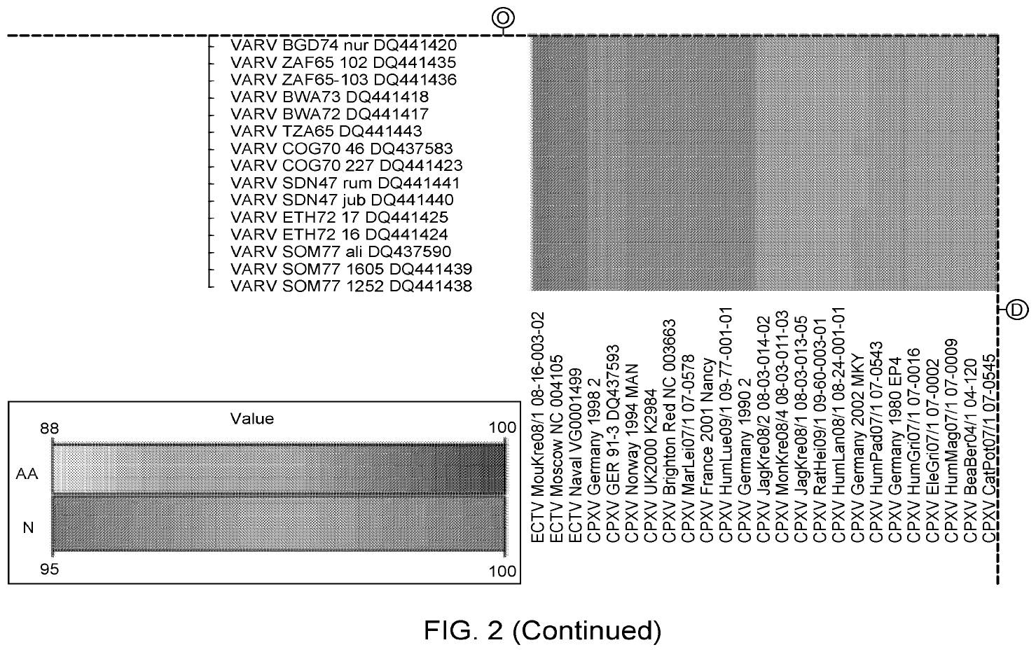

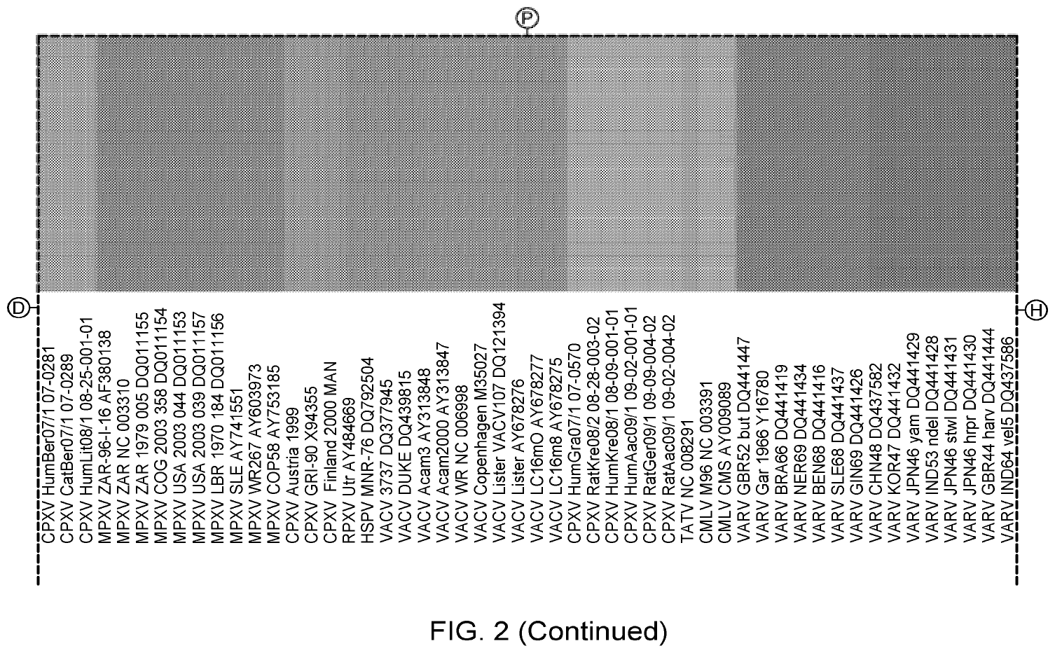

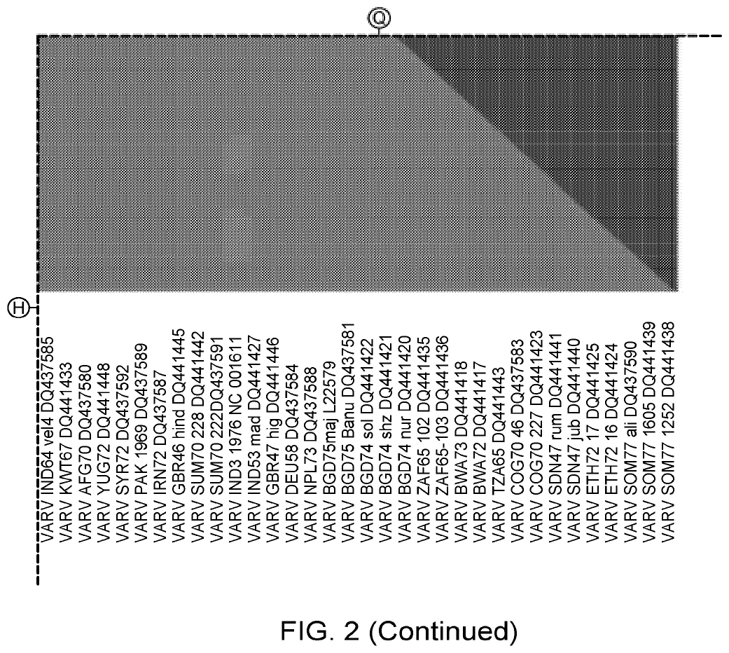

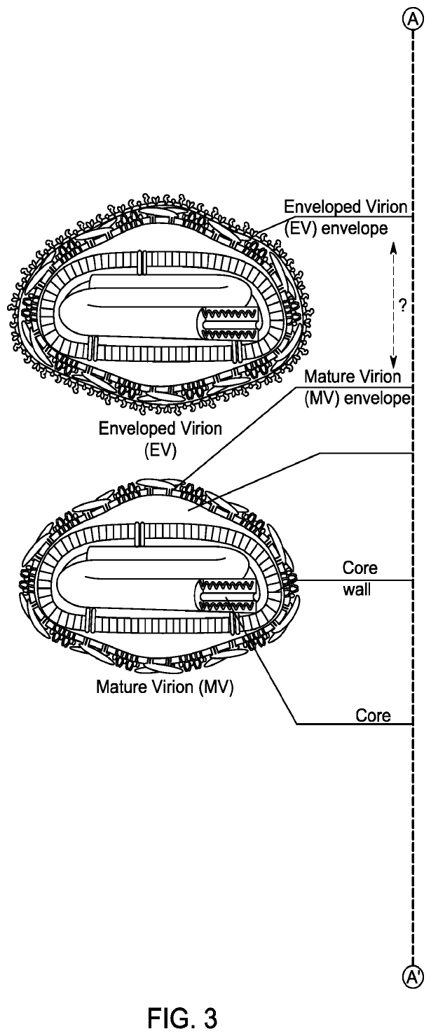

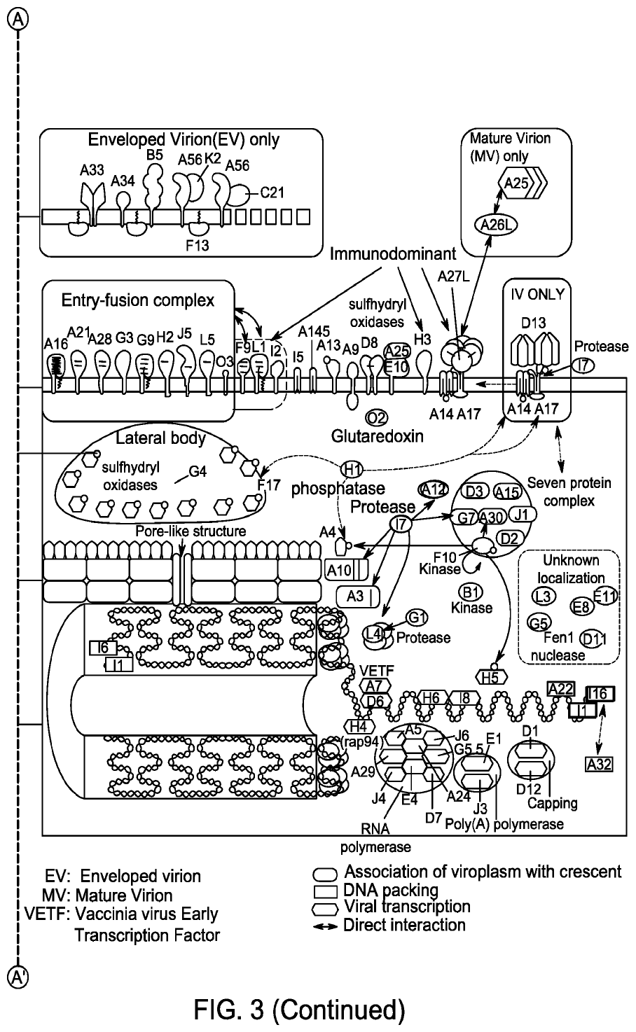

Monkeypox virus (MPXV) is a member of the poxvirus family and orthopoxvirus genus (FIG. 1 and FIG. 2). Orthopoxviruses (OPVs) are characterized by structural and lifecycle complexity (FIG. 3). Orthopoxviruses also include, for example, variola virus (also referred to as smallpox, VARV), vaccinia virus (VACV), and cowpox (CPXV). Monkeypox is closely related to variola virus, and the present disclosure encompasses a recognition that the relatedness of orthopoxviruses is high in general.

Through successful vaccination, smallpox was declared eradicated in 1980. However, since then, the world population has largely become unvaccinated against orthopoxviruses. Low levels of transmission of monkeypox have supported endemicity in West and Central Africa for years, but recently there has been community spread in non-endemic regions including Europe and North America. The waning global population-level immunity against orthopoxviruses is at least partly responsible for this recent spread of monkeypox.

Monkeypox and other orthopoxviruses are a continuing global threat. Variola virus is classified as a category A bioterrorism agent that is especially of concern given the current limitations in supply of canonically produced vaccines. The recent spread of monkeypox also highlights a continuing risk of emergence of a novel orthopoxvirus.

The present disclosure provides orthopox (e.g., monkeypox, variola, vaccinia, or cowpox) polyribonucleotides, antigen constructs, and/or pharmaceutical compositions that are effective for vaccination against orthopox (e.g., monkeypox, variola, vaccinia, or cowpox). The present disclosure also provides the insight that polyribonucleotides, antigen constructs, and pharmaceutical compositions targeting one orthopox species may cross-protect against other orthopoxviruses. For example, in some embodiments, provided monkeypox polyribonucleotides, antigen constructs, and/or pharmaceutical compositions are effective for vaccination against monkeypox and one or more other orthopox viruses. In some embodiments, provided monkeypox polyribonucleotides, antigen constructs, and/or pharmaceutical compositions are effective for vaccination against monkeypox and one or more of variola virus, vaccinia virus, and cowpox. In some embodiments, provided monkeypox polyribonucleotides, antigen constructs, and/or pharmaceutical compositions are effective for vaccination against monkeypox and variola virus. In some embodiments, provided monkeypox polyribonucleotides, antigen constructs, and/or pharmaceutical compositions are effective for vaccination against monkeypox and a novel orthopox virus.

### Monkeypox Structure

Monkeypox virions are ovoid or brick-shaped particles which are enclosed by geometrically corrugated lipoprotein outer membrane. Mature monkeypox virions have a densely packed core containing enzymes, a double-stranded DNA genome, and transcription factors that are protected by a protein core.

The monkeypox genome consists of a linear double-stranded DNA (about 197 kb) covalently joined at its ends by palindromic hairpins, and the inverted terminal repeats (ITRs) are made up of a hairpin loop, tandem repeats, and some open reading frames (ORF). Although MPXV is a DNA virus, its entire life cycle occurs in the cytoplasm of infected cells. All the proteins required for viral DNA replication, transcription, virion assembly, and egress are encoded by the monkeypox genome. The genes encoding for housekeeping functions are highly conserved among OPVs and are present in the central region of the genome while those that encode for the genes mediating virus-host interactions are less conserved and located in the terminal regions of the genome.

In vaccinia virus (and most likely in monkeypox) intracellular mature virus (IMV) and extracellular-enveloped virus (EEV) are two forms of infectious virions produced in poxvirus-infected cells. IMV is released upon cell lysis, while EEV is released from cells via interaction with actin tails, and this is said to be the cause of rapid long distance spread of the virus within the infected host. Although the aforementioned features are for VACV, it is likely that these features are common to all OPVs. Cell-associated virions (CEVs) are formed following the microtubule-mediated transport of intracellular enveloped virus (IEV) to the cell periphery, in which the outer membrane of IEV fuses with the plasma membrane and remains attached to the cell surface. CEVs are mostly responsible for cell-to-cell spread. IEV is formed when IMV is wrapped by a double membrane derived from early endosomal component or the trans-Golgi network (TGN). However, apart from IEV exocytosis, an alternative route for the formation of EEV is by the budding of IMV through the plasma membrane. In the prototype vaccinia virus, virion morphogenesis can be defective resulting in non-infectious dense particles (DPs), but this has not yet been reported for monkeypox. In addition, unlike some strains of CPXV in which IMVs are occluded within A-type inclusions (ATI), monkeypox does not form ATIs or sequester IMVs into ATIs because of truncation in the ATIP gene.

### Monkeypox Transmission

The two possible means of monkeypox transmission are animal-to-human transmission and human-to-human transmission. Respiratory droplets and contact with body fluids, contaminated patient's environment or items, and skin lesion of an infected person have been found to be associated with inter-human transmission. Contact between broken skin or mucous membranes and an infected patient's body fluids, respiratory droplets, or scabs is considered a “high risk” exposure that warrants post-exposure vaccination as soon as possible. Congo Basin (CB) clade (Central Africa clade) is reported to be more virulent than West Africa (WA) clade and thereby contributes more to inter-human transmission. Animal-to-human transmission, which is also known as zoonotic transmission, occurs via direct contact with any of the aforementioned natural viral hosts or consumption of these hosts. In addition, zoonotic transmission could occur by direct contact with the blood, body fluids, and inoculation from mucocutaneous lesions of an infected animal. Nosocomial transmission has been reported for CB and WA clades of monkeypox while sexual transmission has been speculated for infected individuals with groin and genital lesions. At present human-to-animal transmission has not been reported. Human-to-human transmission, secondary attack rates (SARS), and serial transmission events is much higher with the CB clade compared to the WA clade. The reproduction number R0 for the CB clade is estimated to be in the range of 0.6-1.0. The R0 has not be estimated for the WA clade of monkeypoxes, but it is presumed to be lower than that of the CB clade. The upper limit R0 of 1.0 in the CB clade indicates that the viruses will not only sustain human-to-human transmission but may persist in the human population. Presumably, if as expected the R0 of the WA clade is much lower than what was estimated for the CB clade, then sustained human-to-human transmission and persistence in human population are highly unlikely and outbreaks will be largely due to spillover events from zoonotic hosts.

### Monkeypox Treatment

Currently, there are no specific clinically proven treatments for monkeypox infection. As with most viral illnesses, the treatment is supportive symptom management. There are, however, prevention measures that can help prevent and/or reduce severity of an outbreak. Infected individuals should remain in isolation, wear a surgical mask, and keep lesions covered as much as reasonably possible until all lesion crusts have naturally fallen off and a new skin layer has formed. For severe cases, investigational use can be considered for compounds with demonstrated benefit against orthopoxviruses in animal studies and severe vaccinia vaccine complications. The oral DNA polymerase inhibitor brincidofovir, oral intracellular viral release inhibitor tecovirimat, and intravenous vaccinia immune globulin have unknown efficacy against the monkeypox virus. For individuals exposed to the virus, temperature and symptoms should be monitored twice per day for 21 days because that is the accepted upper limit of the monkeypox incubation period. Infectiousness aligns with symptom onset; therefore, close contacts need not isolate while asymptomatic. According to the Centers for Disease Control and Prevention (CDC), vaccination within four days of exposure may prevent disease onset, and vaccination within 14 days may reduce disease severity.

### Exemplary Monkeypox Polypeptides

### B6R

Monkeypox B6R (also referred to as MPXVgp167) is a ˜35 kD polypeptide. B6R is classified as a membrane glycoprotein that is a component of the monkeypox EEV envelope. B6R comprises a transmembrane domain and two sushi domains. B6R is also classified as being involved in negative regulation of complement activation. B6R polypeptide sequences include, e.g., UniProt accession numbers Q8V4S2, V9NQJ0, A0A0F6N8B7, each of which is incorporated herein by reference in its entirety. Exemplary B6R amino acid sequences are provided in Table 1 and Table 8 below.

Monkeypox B6R is homologous to vaccinia B5R. Vaccinia B5R (see, e.g., accession number AAN78219.1) is a membrane protein that is essential in packaging the intracellular mature virion form intracellular enveloped virions, and is EEV-specific. DOIs: https://doi.org/10.1083/jcb.200104124, https://doi.org/10.1128/jvi.68.1.130-147.1994, https://doi.org/10.1099/0022-1317-83-12-2915

### A35R

Monkeypox A35R (also referred to as MPXV-COP-139, MPXV-SL-139, MPXV-WRAIR139) is a ˜20 kD polypeptide. A35R is classified as a membrane protein, specifically a bifunctional EEV membrane phosphoglycoprotein. A35R polypeptide sequences include, e.g., UniProt accession numbers Q8V4U4 and Q80KX2, each of which is incorporated herein by reference in its entirety. Exemplary A35R amino acid sequences are provided in Table 1 and Table 8 below.

Monkeypox A35R is homologous to vaccinia A33R. Vaccinia A33R (see, e.g., accession number AAF63733, incorporated herein by reference in its entirety) is a type II integral membrane protein found in EEV (extracellular enveloped virus) but not IMV, and is highly conserved among orthopoxviruses. See, e.g., DOIs: 10.1128/jvi.72.5.4192-4204.1998 MIR

MPXV M1R (also referred to as IMV membrane protein, MPXV-COP-074, MPXV-SL-074) is a ˜27 kD polypeptide. M1R polypeptide sequences include, e.g., UniProt accession numbers Q8V502, Q80KX3, Q5IXU5, each of which is incorporated herein by reference in its entirety. Exemplary M1R amino acid sequences are provided in Table 1 and Table 8 below.

MPXV M1R is homologous to vaccinia L1R. Vaccinia L1R (see, e.g., accession number AAF63732, incorporated herein by reference in its entirety) is a myristoylated transmembrane protein of about 250 residues that is expressed on the surface of the IMVs. It is considered essential at least in that genetic deletion of L1R renders vaccinia viruses incapable of maturation. L1R appears to be required for maturation of viral particles. See, e.g., DOIs: https://doi.org/10.1128/jvi.68.10.6401-6410.1994, 10.1073/pnas.062163799

### E8L

MPXV E8L (also referred to as cell surface-binding protein, carbonic anhydrase homolog) is a ˜35 kD polypeptide. E8L is a membrane protein. E8L binds to chondroitin sulfate on a target cell surface to provide virion attachment to the target cell. E8L polypeptide sequences include, e.g., UniProt accession numbers A0A0F6N859, Q8V4Y0, Q3I8Q9, Q3I9B0, Q5IXS0, each of which is incorporated herein by reference in its entirety). Exemplary E8L amino acid sequences are provided in Table 1 and Table 8 below.

### H3L

H3L (also referred to as IMV heparin binding surface protein, Envelope protein H3, MPXV-COP-087, MPXV-SL-087, MPXV-WRAIR087, MPXVgp093) is a ˜37.5 kD polypeptide that localizes to the monkeypox viral envelope. H3L polypeptide sequences include, e.g., UniProt accession numbers, Q8V4Z2, Q3I8S1, Q5IXT2, A0A0F6N9X0, each of which is incorporated herein by reference in its entirety). Exemplary H3L amino acid sequences are provided in Table 1 and Table 8 below. Monkeypox H3L was found to bear high sequence similarity to vaccinia H3L.

### A28L

A28L (also referred to as A-type inclusion protein, Cowpox A-type inclusion protein) is a ˜60 kD polypeptide that localizes to the monkeypox viral envelope. A28L is involved in viral entry into host cells and for cell-cell fusion (syncytium formation). Monkeypox A28L sequences include, e.g., UniProt accession number Q8V4V0, V9NSD8, V9NNF4, V9NKU4, V9NWM3, V9NR63, A0A0F7GAP0, A0A2L1F535, A0A0F7G921, A0A0F7GB12, each of which is incorporated herein by reference in its entirety). An exemplary monkeypox A28L amino acid sequence is provided in Table 1 and Table 8 below.

### A29L

A29L (also referred to as 14 kDa protein, 14K membrane protein, IMV surface protein fusion protein, MPXV-COP-132, MPXV-SL-132) is a ˜14 kD polypeptide. A29L localizes to the viral envelope and is involved in fusion of the viral membrane with the host plasma membrane. Monkeypox A29L sequences include, e.g., UniProt accession number Q77HM6, Q9YN60, Q3I824, each of which is incorporated herein by reference in its entirety). Exemplary A29L amino acid sequences are provided in Table 1 and Table 8 below.

Monkeypox A29L is homologous to vaccinia A27L. Vaccinia A27L (see, e.g., accession number AAN78218.2, incorporated herein by reference in its entirety) is implicated in viral attachment, virus-host cell fusion, cell-cell fusion, plaque size and the formation of enveloped virions. See, e.g., Chung et al. J Virol. 1998 February; 72(2):1577-85. doi: 10.1128/JVI.72.2.1577-1585.1998; Doms et al. J Virol. 1990 October; 64(10):4884-92. doi: 10.1128/JVI.64.10.4884-4892.1990; Gong et al. Virology. 1990 September; 178(1):81-91. doi: 10.1016/0042-6822(90)90381-z.; Dallo et al. Virology. 1987 August; 159(2):423-32. doi: 10.1016/0042-6822(87)90481-8; and Payne and Norrby J Gen Virol. 1976 July; 32(1):63-72. doi: 10.1099/0022-1317-32-1-63.

### II. Polyribonucleotides

### A. Exemplary Polyribonucleotides Constructs

The present disclosure, among other things, utilizes RNA technologies as a modality to express one or more orthovirus (e.g., monkeypox) polypeptide constructs that includes one or more orthovirus (e.g., monkeypox) antigens, or one or more portions thereof, described herein.

In some embodiments, the present disclosure provides polyribonucleotides that encode one or more monkeypox antigens or fragments thereof. The present disclosure includes the unexpected discovery that monkeypox B cell antigens provided in Table 1, and fragments thereof, are particularly advantageous for use in preventing or treating monkeypox, e.g., in monkeypox antigen constructs and/or monkeypox vaccines as further disclosed herein.

TABLE 1
Monkeypox B cell antigens

| | Exemplary Amino Acid Sequences | | | | |
|---|---|---|---|---|---|
| | (SEQ ID NOs) | | | | |
| | | Portuguese | Belgian | | |
| | | Outbreak | Outbreak | | |
| | Outbreak | (Portugal | (Belgium | NC_{—} | Further |
| Antigen | Consensus | 20220504) | 20220513) | 003310.1 | Sequences |
| A29L | 1 | 2 | 3 | 4 | 5-10 |
| A35R | 11 | 12 | 13 | 14 | 15-20 |
| B6R | 21 | 22 | 23 | 24 | 25-30 |
| M1R | 31 | 32 | 33 | 34 | 35-40 |
| E8L | 41 | 42 | 43 | 44 | 45-50 |
| H3L | 51 | 52 | 53 | 54 | 55-60 |
| A28L | 196 | 196 | 196 | 196 | |

In various embodiments, a polyribonucleotide of the present disclosure encodes a single monkeypox antigen of Table 1 or fragment thereof (e.g., an A29L polypeptide or fragment thereof, A35R polypeptide or fragment thereof, B6R polypeptide or fragment thereof, M1R polypeptide or fragment thereof, E8L polypeptide or fragment thereof, A28L polypeptide or fragment thereof, or H3L polypeptide or fragment thereof). In some embodiments, a polyribonucleotide of the present disclosure encodes an A29L polypeptide having a sequence of any one of the A29L sequences of Table 1, or fragment thereof. In some embodiments, a polyribonucleotide of the present disclosure encodes an A35R polypeptide having a sequence of any one of the A35R sequences of Table 1, or fragment thereof. In some embodiments, a polyribonucleotide of the present disclosure encodes a B6R polypeptide having a sequence of any one of the B6R sequences of Table 1, or fragment thereof. In some embodiments, a polyribonucleotide of the present disclosure encodes an M1R polypeptide having a sequence of any one of the M1R sequences of Table 1, or fragment thereof. In some embodiments, a polyribonucleotide of the present disclosure encodes an E8L polypeptide having a sequence of any one of the E8L sequences of Table 1, or fragment thereof. In some embodiments, a polyribonucleotide of the present disclosure encodes an H3L polypeptide having a sequence of any one of the H3L sequences of Table 1, or fragment thereof. In some embodiments, a polyribonucleotide of the present disclosure encodes an A28L polypeptide having a sequence of any one of the A28L sequences of Table 1, or fragment thereof.

In various embodiments, a polyribonucleotide of the present disclosure encodes two, three, four, five or six monkeypox antigens of Table 1 or fragments thereof. In some embodiments, a polyribonucleotide of the present disclosure encodes two or more monkeypox polypeptides selected from: an E8L polypeptide or fragment thereof, an A35R polypeptide or fragment thereof, a B6R polypeptide or fragment thereof, an MIR polypeptide or fragment thereof, an H3L polypeptide or fragment thereof, an A28L polypeptide or fragment thereof, and an A29L polypeptide or fragment thereof.

In some embodiments, a polyribonucleotide of the present disclosure encodes a B6R polypeptide having a sequence of any one of the B6R sequences of Table 1, and an MIR polypeptide having a sequence of any one of the MIR sequences of Table 1. In some embodiments, a polyribonucleotide of the present disclosure encodes an A35R polypeptide having a sequence of any one of the A35R sequences of Table 1, a B6R polypeptide having a sequence of any one of the B6R sequences of Table 1, and an MIR polypeptide having a sequence of any one of the MIR sequences of Table 1. In some embodiments, a polyribonucleotide of the present disclosure encodes an A35R polypeptide having a sequence of any one of the A35R sequences of Table 1, a B6R polypeptide having a sequence of any one of the B6R sequences of Table 1, an MIR polypeptide having a sequence of any one of the MIR sequences of Table 1, and an E8L polypeptide having a sequence of any one of the E8L sequences of Table 1. In some embodiments, a polyribonucleotide of the present disclosure encodes an A35R polypeptide having a sequence of any one of the A35R sequences of Table 1, a B6R polypeptide having a sequence of any one of the B6R sequences of Table 1, an MIR polypeptide having a sequence of any one of the MIR sequences of Table 1, and an H3L polypeptide having a sequence of any one of the H3L sequences of Table 1.

In some embodiments, a polyribonucleotide of the present disclosure encodes one or more IMV antigens. In some embodiments, one or more IMV antigens are selected from H3L, E8L, MIR, and A29L. In some embodiments, a polyribonucleotide of the present disclosure encodes one or more IMV-specific antigens. In some embodiments, one or more IMV-specific antigens are selected from H3L, E8L, MIR, and A29L.

In some embodiments, a polyribonucleotide of the present disclosure encodes one or more EEV antigens. In some embodiments, one or more EEV antigens are selected from A35R and B6R. In some embodiments, a polyribonucleotide of the present disclosure encodes one or more EEV-specific antigens. In some embodiments, one or more EEV-specific antigens are selected from A35R and B6R.

In some embodiments, a polyribonucleotide of the present disclosure encodes one or more E8L polypeptides (e.g., an E8L antigen or one or more fragments thereof). In some embodiments, a polyribonucleotide of the present disclosure encodes one or more A35R polypeptides (e.g., an A35R antigen or one or more fragments thereof). In some embodiments, a polyribonucleotide of the present disclosure encodes one or more B6R polypeptides (e.g., a B6R antigen or one or more fragments thereof). In some embodiments, a polyribonucleotide of the present disclosure encodes one or more M1R polypeptides (e.g., an M1R antigen or one or more fragments thereof). In some embodiments, a polyribonucleotide of the present disclosure encodes one or more H3L polypeptides (e.g., an H3L antigen or one or more fragments thereof). In some embodiments, a polyribonucleotide of the present disclosure encodes one or more A28L polypeptides (e.g., an A28L antigen or one or more fragments thereof). In some embodiments, a polyribonucleotide of the present disclosure encodes one or more A29L polypeptides (e.g., an A29L antigen or one or more fragments thereof).

The present disclosure includes the unexpected discovery that monkeypox T cell antigens provided in Table 2, and fragments thereof, are particularly advantageous for use in preventing or treating monkeypox, e.g., in monkeypox antigen constructs and/or monkeypox vaccines as further disclosed herein.

TABLE 2
Monkeypox T cell antigens

| | Exemplary Amino Acid Sequences | | | | |
|---|---|---|---|---|---|
| | (SEQ ID NOs) | | | | |
| | | Portuguese | Belgian | | |
| | | Outbreak | Outbreak | | |
| | Outbreak | (Portugal | (Belgium | NC_{—} | Further |
| Antigen | Consensus | 20220504) | 20220513) | 003310.1 | Sequences |
| A45L | 61 | 62 | 63 | 64 | 65 |
| B9R | 66 | 67 | 68 | 69 | 70-75 |
| B16R | 76 | 77 | 78 | 79 | 80-85 |
| C10L | 86 | 87 | 88 | 89 | 90-91 |
| C21L | 92 | 93 | 94 | 95 | 96 |
| E7R | 97 | 98 | 99 | 100 | 101 |
| F3L | 102 | 103 | 104 | 105 | 106-111 |
| F4L | 112 | 113 | 114 | 115 | 116 |
| G6R | 117 | 118 | 119 | 120 | 121 |
| H5R | 122 | 123 | 124 | 125 | 126 |
| I3L | 127 | 128 | 129 | 130 | 131 |
| O2L | 132 | 133 | 134 | 135 | 136 |
| Q1L | 137 | 138 | 139 | 140 | 141 |
| B12R | 244 | 245 | 246 | 247 | |
| C17L | 248 | 249 | 250 | 251 | |

In various embodiments, a polyribonucleotide of the present disclosure encodes a single monkeypox antigen of Table 2 or fragment thereof (e.g., an A45L polypeptide or fragment thereof, B9R polypeptide or fragment thereof, B16R polypeptide or fragment thereof, C10L polypeptide or fragment thereof, C21L polypeptide or fragment thereof, E7R polypeptide or fragment thereof, F3L polypeptide or fragment thereof, F4L polypeptide or fragment thereof, G6R polypeptide or fragment thereof, H5R polypeptide or fragment thereof, I3L polypeptide or fragment thereof, O2L polypeptide or fragment thereof, Q1L polypeptide or fragment thereof, B12R polypeptide or fragment thereof, or C17L polypeptide or fragment thereof). In various embodiments, a polyribonucleotide of the present disclosure encodes a plurality of monkeypox antigens of Table 2 or fragments thereof (e.g., 1, 2, 3, 4, 5, 6, 7, 8, 9, 10, 11, 12, 13, 14, or 15 monkeypox antigens of Table 2 or fragments thereof). In various embodiments, a polyribonucleotide of the present disclosure encoding a plurality of monkeypox antigens or fragments thereof (e.g., monkeypox antigens of Table 2 or fragments thereof) can be referred to as a string construct.

The present disclosure includes, among other things, the recognition that in some embodiments it may be particularly advantageous to include a string of monkeypox T cell antigens of Table 2, or fragments thereof in a single polyribonucleotide. The present disclosure further includes, among other things, the recognition that in some embodiments it may be particularly advantageous to combine, in a composition or use, at least a first polyribonucleotide encoding a monkeypox B cell antigen or fragment thereof (e.g., an antigen of Table 1 or a fragment thereof) and a second polyribonucleotide that encodes a plurality of monkeypox T cell antigens or fragments thereof (e.g., antigens of Table 2 or fragments thereof).

As described herein, in some embodiments, provided technologies involve administration of a plurality of antigens to the same subject. In some embodiments, multiple antigens are administered at the same time (e.g., in a single dose). In some embodiments, different antigens may be administered at different times (for example in different doses—e.g., a prime dose vs a boost dose). In some embodiments, multiple antigens are administered via the same composition.

For clarity, a single “antigen” polypeptide may include multiple “epitopes”, which in turn may or may not be linked with one another in nature. For example, a single string construct antigen includes multiple epitopes, which may be from different parts of the same monkeypox protein and/or from different monkeypox proteins, linked together as described herein in a single polypeptide.

Thus, a single pharmaceutical composition (e.g., immunogenic composition, e.g., vaccine) as described herein may include or deliver (e.g., because the pharmaceutical composition includes a nucleic acid, such as an RNA, that encodes the antigen and is expressed upon administration) a single antigen, which itself may comprise multiple epitopes (either in their natural arrangement relative to one another or in an engineered or constructed arrangement as described herein), or may comprise or deliver a plurality of antigens, each of which similarly may be or comprise a single epitope or multiple epitopes (either in their natural arrangement relative to one another or in an engineered or constructed arrangement as described herein). Still further, a single pharmaceutical composition (e.g., immunogenic composition, e.g., vaccine) may, for example, include multiple distinct nucleic acids (e.g., RNAs) that each encode different antigen(s) or, in some embodiments, may include a single nucleic acid that encodes (and expresses) multiple antigens. Yet further, a single pharmaceutical composition (e.g., immunogenic composition, e.g., vaccine) that includes multiple distinct nucleic acids (e.g., RNAs) encoding antigens may, in some embodiments, be prepared by mixing the RNAs and then incorporating the mixture into LNPs, or alternatively by formulating individual RNAs into LNPs and then mixing the LNPs. In some embodiments, mixtures (whether of RNAs pre-LNP preparation or of LNPs) may include the relevant RNAs in 1:1 ratio, or in other ratios as may be preferred (e.g., to achieve a desired relative presentation of antigens or epitopes) in a subject to whom the composition is administered.

In some embodiments, two or more RNA molecules each encoding a different polypeptide (e.g., a monkeypox antigen as described herein) can be mixed with particle-forming agents to form nucleic acid containing particles as described above. In alternative embodiments, two or more RNA molecules each encoding a different polypeptide (e.g., a monkeypox antigen as described herein) can be formulated into separate particle compositions, which are then mixed together. For example, in some embodiments, individual populations of nucleic acid containing particles, each population comprising an RNA molecule encoding a different immunogenic polypeptide or immunogenic fragment thereof (e.g., a monkeypox antigen as described herein), can be separately formed and then mixed together, for example, prior to filling into vials during a manufacturing process, or immediately prior to administration (e.g., by an administering health-care professional)). Accordingly, in some embodiments, described herein is a composition comprises two or more populations of particles (e.g., in some embodiments, lipid nanoparticles), each population comprising at least one RNA molecule encoding a different immunogenic polypeptide or immunogenic fragment thereof (e.g., monkeypox antigen or fragment thereof). In some embodiments, each population may be provided in a composition at a desirable proportion (e.g., in some embodiments, each population may be provided in a composition in an amount that provides the same amount of RNA molecules).

In some embodiments, one or more pharmaceutical compositions (e.g., immunogenic composition, e.g., vaccine) may comprise or deliver a combination comprising a polypeptide or portion thereof encoded by all or part of A29L, A35R, B6R, M1R, E8L, A28L, H3L, A45L, B9R, B16R, C10L, C21L, E7R, F3L, F4L, G6R, H5R, I3L, O2L, Q1L, B12R, and/or C17L or fragments thereof.

In some embodiments, one or more pharmaceutical compositions (e.g., immunogenic composition, e.g., vaccine) may comprise or deliver a combination comprising a polypeptide or portion thereof encoded by all or part of E8L, A35R, B6R, M1R, H3L, A28L, A29L, and/or fragments of any thereof.

In some embodiments, one or more pharmaceutical compositions (e.g., immunogenic composition, e.g., vaccine) comprise or deliver a combination comprising one or more IMV antigens (e.g., IMV-specific antigens) and one or more EEV antigens (e.g., EEV-specific antigens). In some embodiments, one or more pharmaceutical compositions comprise or deliver a combination of monkeypox antigens that includes (i) one or more IMV antigens (e.g., IMV-specific antigens) selected from H3L, E8L, M1R, A29L, and fragments of any thereof; and (ii) one or more EEV antigens (e.g., EEV-specific antigens) selected from A35R, B6R, and fragments thereof.

In some embodiments, one or more pharmaceutical compositions (e.g., immunogenic composition, e.g., vaccine) may comprise or deliver a combination comprising a polypeptide or portion thereof encoded by all or part of B6R, M1R, and/or fragments of any thereof. In some embodiments, one or more pharmaceutical compositions (e.g., immunogenic composition, e.g., vaccine) may comprise or deliver a combination comprising a polypeptide or portion thereof encoded by all or part of B6R, A35R, and/or fragments of any thereof. In some embodiments, one or more pharmaceutical compositions (e.g., immunogenic composition, e.g., vaccine) may comprise or deliver a combination comprising a polypeptide or portion thereof encoded by all or part of B6R, A35R, M1R, and/or fragments of any thereof. In some embodiments, one or more pharmaceutical compositions (e.g., immunogenic composition, e.g., vaccine) may comprise or deliver a combination comprising a polypeptide or portion thereof encoded by all or part of B6R, A35R, M1R, H3L, and/or fragments of any thereof. In some embodiments, one or more pharmaceutical compositions (e.g., immunogenic composition, e.g., vaccine) may comprise or deliver a combination comprising a polypeptide or portion thereof encoded by all or part of B6R, A35R, M1R, E8L, and/or fragments of any thereof. In some embodiments, one or more pharmaceutical compositions (e.g., immunogenic composition, e.g., vaccine) may comprise or deliver a combination comprising a polypeptide or portion thereof encoded by all or part of B6R, A35R, M1R, H3L, E8L, and/or fragments of any thereof.

In some embodiments, a provided composition includes or delivers a monkeypox envelope glycoprotein antigen (e.g., a full-length monkeypox envelope glycoprotein, a fragment thereof, or one or more epitopes thereof, for example in a string construct). In some embodiments, a provided composition includes or delivers such a monkeypox envelope glycoprotein antigen together with one or more B cell targets (e.g., epitopes) which may, for example, be or comprise one or more other monkeypox proteins (or fragments or epitopes thereof). In some embodiments, such a B cell target is or comprises a monkeypox protein (or fragment or epitope thereof) that is predicted or known to induce a B cell response in infected humans. For example, in some embodiments, a B cell target is or comprises a monkeypox protein (or fragment or B cell epitope thereof) against which sera from infected individual(s) is reactive. In some particular embodiments, a B cell target is or comprises a monkeypox envelope glycoprotein, or other relevant monkeypox protein, or a fragment or epitope thereof.

In some embodiments, a provided composition comprises or delivers a string construct antigen that includes a plurality of T cell epitopes, optionally from more than one monkeypox protein. In some such embodiments, a provided composition further comprises or delivers one or more B cell targets. Alternatively or additionally, in some embodiments, a string construct antigen so utilized includes monkeypox sequences (e.g., one or more fragments or epitopes, e.g., T cell epitopes and/or B cell epitopes, but in some embodiments specifically T cell epitopes).

In some embodiments, a string construct antigen includes both B cell epitopes and T cell epitopes (optionally from the same monkeypox protein or from different monkeypox proteins).

In some embodiments, different antigens may be delivered by administration of different compositions, which in turn may, in some embodiments, be administered at the same time (e.g., as an admixture or otherwise substantially simultaneously) and, in some embodiments, may be administered at different times. To give but one example, in some embodiments, a particular antigen or antigen(s) may be delivered via an initial pharmaceutical composition (e.g., immunogenic composition, e.g., vaccine) dose, and one or more other antigen(s) may be delivered via one or more booster dose(s).

In some embodiments, an antigen utilized (i.e., included in and/or otherwise delivered by) a pharmaceutical composition (e.g., immunogenic composition, e.g., vaccine) described herein comprises multiple epitopes, e.g., of a single monkeypox protein or of multiple proteins.

In some embodiments, an antigen may comprise two or more epitopes from the same monkeypox protein and in their natural configuration relative to one another (e.g., in a fragment if the relevant protein). In some embodiments, however, an antigen may comprise at least two epitopes configured in a non-natural relationship relative to one another (e.g., included in a string construct as described herein.

Among other things, the present disclosure provides an insight that string construct antigens may be particularly useful or effective for vaccination against a monkeypox infection. Without wishing to be bound by any particular theory, the present disclosure proposes that ability to link individual epitopes predicted or determined to have specific attributes—e.g., binding to relevant HLA alleles, expression at relevant times of infection, representation of particularly conserved sequences, potentially across a plurality of different monkeypox proteins, may prove uniquely beneficial, or indeed critical, for effective vaccination against monkeypox, where more traditional vaccination approaches have thus far provided only limited protection.

In some embodiments, a multi-epitope antigen (e.g., a string construct antigen or a polyepitopic antigen) may be administered as a polypeptide and/or as a collection of peptides. Alternatively or additionally, a multi-epitope antigen may be administered as preparation of cells that comprise (e.g., express) the antigen. However, the present disclosure further provides an insight that, in some embodiments, delivery by administration of a nucleic acid, and particularly of an RNA, encoding the multi-epitope antigen, may be particularly useful and/or effective.

Experience with SARS-CoV-2 has demonstrated that RNA administration can be a particularly effective way to deliver an infectious disease antigen. Furthermore, the present disclosure provides an insight that various features of nucleic acid formats including, for example their flexibility and amenability to rapid design and modification, including incorporation of a variety of insights (e.g., bioinformatics inputs etc), renders them particularly attractive for use in a monkeypox vaccine. Among other things, the present disclosure provides an insight that, in some embodiments, administration of an RNA encoding a string construct antigen as described herein may be a particularly desirable and/or effective approach to immunizing against monkeypox infection.

In some embodiments, a “string” polynucleotide sequence encodes a plurality of antigens and/or epitopes in tandem. In some embodiments, a string encodes about 2 to about 100, about 2 to about 75, about 2 to about 50, about 2 to about 25, about 2 to about 20, about 2 to about 15, about 2 to about 10, or about 2 to about 5 antigens and/or epitopes. In some embodiments, a string encodes about 5 to about 100, about 5 to about 75, about 5 to about 50, about 5 to about 25, about 5 to about 20, about 5 to about 15, or about 5 to about 10 antigens and/or epitopes. In some embodiments, a “string” polynucleotide sequence encodes a plurality of epitopes in tandem. In some embodiments, a string encodes about 2 to about 1000 or about 2 to about 10,000 antigens and/or epitopes. In some embodiments about 2-5,000 antigens and/or epitopes are encoded in one polynucleotide string. In some embodiments, about 2-4,000 antigens and/or epitopes are encoded in one polynucleotide string. In some embodiments, about 2-3,000 antigens and/or epitopes are encoded in one polynucleotide string. In some embodiments about 2-2,000 antigens and/or epitopes are encoded in one polynucleotide string. In some embodiments, about 2-1,000 antigens and/or epitopes are encoded in one polynucleotide string. In some embodiments, about 10-500 antigens and/or epitopes are encoded in one polynucleotide string. In some embodiments, about 10-200 antigens and/or epitopes are encoded in one polynucleotide string. In some embodiments, about 20-100 antigens and/or epitopes are encoded in one polynucleotide string.

In some embodiments, epitopes encoded by string constructs comprise epitopes that are predicted by a HLA binding and presentation prediction software to be of high likelihood to be presented by a protein encoded by an HLA to a T cell for eliciting an immune response. In some embodiments, epitopes that are predicted to have a high likelihood to be presented by a protein encoded by an HLA, are selected from any one of the proteins or peptides described in Table 1 or Table 2. In some embodiments, epitopes encoded by a string construct comprise membrane-associated or otherwise accessible epitopes, e.g., at relevant time(s) during the monkeypox life cycle.

In some embodiments, an antigen utilized in accordance with the present disclosure is or comprises A29L, A35R, B6R, M1R, E8L, A28L, H3L, A45L, B9R, B16R, C10L, C21L, E7R, F3L, F4L, G6R, H5R, I3L, O2L, Q1L, B12R, and/or C17L or fragments thereof, variants thereof and/or fragments or epitopes of any of the foregoing, or a combination of any of the foregoing. In some embodiments, an antigen utilized in accordance with the present disclosure is or comprises a monkeypox protein, a monkeypox envelope protein, a monkeypox tegument protein, a monkeypox membrane protein, variants thereof and/or fragments or epitopes of any of the foregoing, or a combination of any of the foregoing. In some embodiments, a string construct may encode a multitude of epitopes that are from 2, 3, 4, or more monkeypox proteins. In some embodiments a string construct comprises one or more features described herein, including the examples and tables. In some embodiments the string construct encodes one or more antigens and/or epitopes comprising one or more sequences of Table 1 or Table 2, or fragments thereof.

Alternatively or additionally, in some embodiments, one or more string constructs may encode one or more other epitopes (e.g., as may be predicted or demonstrated, for example in literature). In some embodiments, a string construct may comprise sequences encoding features such as linkers, and cleavage sites (e.g., auto-cleavage sites such as, for example, T2A, or P2A sequences). In some embodiments, a linker that is enriched in G and S residues can be used. In some embodiments, an exemplary linker may have a sequence of GGGGSGGGGS (SEQ ID NO: 222) or GGSGGGGSGG (SEQ ID NO: 176).

In some embodiments, a string construct comprises two or more overlapping epitope-coding sequences.

In some embodiments, a string construct comprises a nucleic acid sequence encoding a polypeptide comprising an amino acid sequence that is 80%, 81%, 82%, 83%, 84%, 85%, 86%, 87%, 88%, 89%, 90%, 91%, 92%, 93%, 94%, 95%, 96%, 97%, 98%, 99% or 100% identical to any one of the sequences in Table 1 or Table 2. As noted above, where sequences being compared are longer than about 20 amino acids, percent identity or homology is typically greater than about 80%; for sequences longer than about 50 amino acids, percent identity or homology is typically greater than about 90%.

In some embodiments, epitopes are arranged and/or encoded on a string to maximize immunogenicity of the expressed epitopes, for example by maximizing recognition by HLA allele repertoire of a subject. In some embodiments, the same string encodes epitopes that can bind to and/or are predicted to bind to different HLA alleles. For instance, as is well exemplified in the sequences tables, e.g., at least in Table 1 and Table 2, a string may encode: (a) a first epitope that binds to or is predicted to bind to a first MHC peptide encoded by a first HLA allele; (b) a second epitope that binds to or is predicted to bind to a second MHC peptide encoded by a second HLA allele; (c) a third epitope that binds to or is predicted to bind to a third MHC peptide encoded by a third HLA allele—and more such epitopes can be added, as in for example in string sequences as provided herein; wherein the first, second and third epitopes are epitopes from the same monkeypox protein, or from different monkeypox proteins. In this way, epitope distribution encoded by a single string is maximized for hitting the different MHC based presentation to T cells, thereby maximizing the probability of generating a desired immune response from a wider range of patients in the given population and maximizing the robustness of the response of each patient.

In some embodiments, epitopes included in a string construct are selected on the basis of high scoring prediction for binding to an HLA by a reliable prediction algorithm or system, such as the RECON prediction algorithm. In some embodiments, the present disclosure provides an insight that particularly successful strings can be provided by: selecting epitopes based on highly reliable and efficient prediction algorithms, arranging the layout of the epitopes encoded by the string; including or omitting non-epitope sequences or sequences flanking the epitopes, validating the immunogenicity of the string in an ex vivo cell culture model, or in an animal model, specifically showing T cell induction following vaccination with a string construct or a polypeptide encoded by a string construct, selecting strings eliciting a specific T cell response, or a combination thereof. In some embodiments, validation may be from use in human patients, and a finding that T cells obtained from a patient post vaccination show an efficient and lasting epitope-specific T cell response. In some embodiments, efficiency of a string as a vaccine is influenced by its design that in part depends on strength of the bioinformatics information used in the thoughtful execution of the design, the reliability of the MHC presentation prediction model, the efficiency of epitope processing when a string vaccine is expressed in a cell, or combinations thereof, among others.

In some embodiments a multi-epitopic RNA (e.g., mRNA) construct as described above comprises 1, 2, 3, 4, 5, 6, 7, 8, 9, 10, 11, 12, 13, 14, 15 or more antigens and/or epitopes. In some embodiments, a pharmaceutical composition comprises 2, 3, 4, 5, 6, 7, 8, 9, or 10 or more strings. In some embodiments, a pharmaceutical composition comprises 6 strings. In some embodiments, a pharmaceutical composition comprises 7 strings. In some embodiments, a pharmaceutical composition comprises 8 strings. In some embodiments, a pharmaceutical composition comprises 9 strings. In some embodiments, a pharmaceutical composition comprises 10 strings.

In some embodiments, epitope-coding sequences in a string construct are flanked by one or more sequences selected for higher immunogenicity, better cleavability for peptide presentation to MHCs, better expression, and/or improved translation in a cell in a subject. In some embodiments, flanking sequences comprise a linker with a specific cleavable sequences. In some embodiments, epitope-coding sequences in a string construct are flanked by a secretory protein sequence.

In some embodiments, a string sequence encodes an epitope that may comprise or otherwise be linked to a signal sequence, such as those listed in Table 3, or at least a sequence having 1, 2, 3, 4, or 5 amino acid differences relative thereto. In some embodiments, a string sequence encodes an epitope that may comprise or otherwise be linked to a signal sequence such as MFVFLVLLPLVSSQCVNLT (SEQ ID NO: 146), or at least a sequence having 1, 2, 3, 4, or at the most 5 amino acid differences relative thereto. In some embodiments, a string sequence encodes an epitope that may be linked at the N-terminal end by a sequence that is enriched in G and S residues, or a sequence having 1, 2, 3, 4, or at the most 5 amino acid differences relative thereto. In some embodiments, an exemplary linker that may be useful to link epitopes has a sequence of GGSGGGGSGG (SEQ ID NO: 176).

In some embodiments, linked sequences may comprise a linker with a cleavage sequence, e.g., with specific cleavable sequences.

In some embodiments, a string construct is linked to a transmembrane domain (TM) or other membrane-associating element. In some embodiments, a linker may have a length of 2, 3, 4, 5, 6, 7, 8, 9, 10 or more amino acid. In some embodiments, a linker of not more than about 30, 25, 20, 15, 10 or fewer amino acids is used. In some embodiments, a linker sequence is not limited to comprise any particular amino acids; in some embodiments, a linker sequence comprises any amino acids. In some embodiments, a linker or cleavage sequence comprises glycine (G). In some embodiments, a linker or cleavage sequence comprises serine (S). In some embodiments, a linker is designed to comprise amino acids based on a cleavage predictor to generate highly-cleavable sequences peptide sequences, and is a novel and effective way of delivering immunogenic T cell epitopes in a T cell vaccine setting.

In some embodiments, epitope distribution and their juxtaposition encoded in a string construct are so designed to facilitate cleavage sequences contributed by the amino acid sequences of the epitopes and/or the flanking or linking residues and thereby using minimal linker sequences. Some exemplary cleavage sequences, without limitation, may be one or more of FRAC, KRCF, KKRY, ARMA, RRSG, MRAC, KMCG, ARCA, KKQG, YRSY, SFMN, FKAA, KRNG, YNSF, KKNG, RRRG, KRYS, and ARYA (SEQ ID NOs: 223-240, respectively).

In some embodiments, a string construct is RNA (e.g., mRNA). In some embodiments, a pharmaceutical composition comprises one or more RNA (e.g., mRNA) string constructs, each comprising a sequence encoding a plurality of epitopes as described herein. In some embodiments, the one or more RNA (e.g., mRNA) comprises a plurality of epitopes, wherein each of the plurality of epitopes is predicted by an HLA binding and presentation prediction algorithm to be of high likelihood to be presented by a protein encoded by an HLA to a T cell for eliciting immune response.

In some embodiments, one or more RNAs (e.g., mRNAs) utilized in a pharmaceutical composition (e.g., immunogenic composition, e.g., vaccine) as described herein encodes a plurality of epitopes (e.g., including one or more, or two or more, sequences provided in Table 1 or Table 2, or fragments thereof), optionally wherein each of the plurality is predicted by an HLA binding and presentation prediction algorithm to be of high likelihood to be presented by a protein encoded by an HLA to a T cell for eliciting an immune response. In some embodiments, the plurality of epitopes comprises epitopes from a single monkeypox protein. In some embodiments, the plurality of epitopes comprises epitopes from multiple monkeypox proteins.

In some embodiments, one or more RNAs (e.g., mRNAs) utilized in a pharmaceutical composition (e.g., immunogenic composition, e.g., vaccine) as described herein include a first RNA that encodes a monkeypox antigen expressed prior to cell infiltration or infection and includes one or more portions expected or known to interface with host cytoplasm. In some embodiments, a monkeypox antigen encoded by a first RNA is or comprises a monkeypox antigen, fragment, or epitope, e.g., a A29L, A35R, B6R, M1R, E8L, A28L, H3L, A45L, B9R, B16R, C10L, C21L, E7R, F3L, F4L, G6R, H5R, 13L, O2L, Q1L, B12R, and/or C17L or fragments thereof, epitopes thereof, and/or a combination thereof. In some embodiments, one or more RNAs (e.g., mRNAs) utilized in a pharmaceutical composition (e.g., immunogenic composition, e.g., vaccine) as described herein includes a second antigen RNA that encodes a multi-epitopic (e.g., polyepitopic) antigen. In some embodiments, a multi-epitopic antigen comprises two or more antigens found in Table 1 or Table 2, or fragments thereof or epitopes thereof. In some embodiments, a multi-epitopic antigen comprises two or more antigens listed in Table 1 or Table 2, and/or fragments and/or epitopes thereof.

In some embodiments, one or more RNAs (e.g., mRNAs) utilized in a pharmaceutical composition (e.g., immunogenic composition, e.g., vaccine) as described herein includes a plurality of epitopes that are predicted by an HLA binding and presentation prediction algorithm to be of high likelihood to be presented by a protein encoded by an HLA to a T cell for eliciting an immune response. In some embodiments, the plurality of epitopes comprises epitopes from a single monkeypox protein. In some embodiments, the plurality of epitopes comprises epitopes from multiple monkeypox proteins.

In some embodiments, a string construct can include an internal ribosome entry site (IRES), e.g., between two sequences encoding antigens or fragments thereof engineered for expression from a polyribonucleotide as distinct polypeptides. Internal ribosome entry sites (IRESs) are cis-acting elements that can recruit the small ribosomal subunits to an internal initiator codon in a polyribonucleotide in conjunction with cellular trans-acting factors. The ability of internal ribosome entry site (IRES) elements to promote internal initiation of translation of polyribonucleotide sequences can facilitate or permit expression of two or more polypeptides from a polycistronic polyribonucleic acid. An exemplary IRES is the encephalomyocardiris virus (EMCV) IRES.

In some embodiments, a multi-epitope polyribonucleotide encoding a super-motif-bearing or motif-bearing polypeptide, together with a helper epitope (e.g., a heterologous helper epitope) and an endoplasmic reticulum-translocating signal sequence. See, for example, in An & Whitton J. Virol. 71:2292, 1997; Thomson. et al., J. Immunol. 157:822, 1996; Whitton, et al., J. Virol 67:348, 1993; Hanke, et al., Vaccine 16:426, 1998.

Additionally, polyribonucleotides described herein, in some embodiments, include other elements such as described below, including, a secretion signal-encoding region, a 5′ Cap, a Cap proximal sequence, a 5′ UTR, a 3′ UTR, and/or a polyA tail. In some embodiments, polyribonucleotides described herein can comprise a secretion signal-encoding region. In some embodiments, epitopes encoded in a string construct may be flanked by a signal peptide sequence, e.g., SP1 sequence (HSV-1 gD signal peptide/secretory domain, SEQ ID NO: 143). In some embodiments, polyribonucleotides described herein can comprise a nucleotide sequence that encodes a 5′UTR of interest and/or a 3′ UTR of interest. In some embodiments, a polynucleotide comprises a dEarI-hAg sequence (SEQ ID NO: 155). In some embodiments, the RNA (e.g., mRNA) comprises a 5′UTR and a 3′UTR. In some embodiments, a 3′UTR comprises a poly A sequence. In some embodiments, a poly A sequence comprises between 50-200 nucleotides. In some embodiments, a poly A tail of a string construct may comprise about 150 A residues. In some embodiments, a poly A tail may comprise 120 residues or less. In some embodiments, a poly A tail of a string construct may comprise about 120 A residues. In some embodiments, a poly A tail of a string construct may comprise about 100 A residues. In some embodiments, a poly A tail of a string construct comprises a “split” or “interrupted” poly A tail (e.g., as described in WO2016/005324). In some embodiments, polyribonucleotides described herein may comprise a 5′ cap, which may be incorporated during transcription, or joined to a polyribonucleotide post-transcription.

### 1. Secretion Signals

In some embodiments, a polyribonucleotide described herein comprises a sequence encoding a human secretion signal. For example, in some embodiments, such a human secretion signal may be or comprises the amino acid sequence of MDWIWRILFLVGAATGAHS (husec2; SEQ ID NO: 142). In some embodiments, a ribonucleic acid sequence encoding a secretion signal included in a polyribonucleotide consists of or comprises a nucleotide sequence that encodes a non-human secretion signal. In some embodiments, a polyribonucleotide encodes a human secretion signal where the secretion signal comprises the amino acid sequence MDWIWRILFLVGAATGAHS (husec2; SEQ ID NO: 142).

In some embodiments, an RNA sequence encodes an antigen or fragment thereof that may comprise or otherwise be linked to a signal sequence (e.g., a secretory sequence), such as those listed in Table 3, or a sequence having 1, 2, 3, 4, or 5 amino acid differences relative thereto. In some embodiments, a signal sequence such as MRVMAPRTLILLLSGALALTETWAGS (SEQ ID NO: 157), or a sequence having 1, 2, 3, 4, or at the most 5 amino acid differences relative thereto is utilized.

In some embodiments, a signal sequence is selected from those included in Table 3 below:

TABLE 3
Exemplary Signal sequences

| Signal | Sequence (Amino Acid) | SEQ ID NO. |
|---|---|---|
| HSV-1 gD SP | MGGAAARLGAVILFVVIVGLHGVRSKY | 143 |
| HSV-2 gD SP | MGRLTSGVGTAALLVVAVGLRVVCA | 144 |
| HSV-2 | MGRLTSGVGTAALLVVAVGLRVVCAKYA | 145 |
| SARS-CoV-2-S | MFVFLVLLPLVSSQCVNLT | 146 |
| human Ig heavy | MDWIWRILFLVGAATGAHSQM | 147 |
| chain signal | | |
| peptide | | |
| human Ig heavy | MDWTWRVFCLLAVAPGAHS | 148 |
| chain signal | | |
| peptide | | |
| HulgGk signal | METPAQLLFLLLLWLPDTTG | 149 |
| peptide | | |
| IgE heavy chain | MDWTWILFLVAAATRVHS | 150 |
| epsilon-1 signal | | |
| peptide | | |
| Japanese | MLGSNSGQRVVFTILLLLVAPAYS | 151 |
| encephalitis | | |
| PRM signal | | |
| sequence | | |
| VSVg protein | MKCLLYLAFLFIGVNCA | 152 |
| signal sequence | | |
| JEV signal | MWLVSLAIVTACAGA | 153 |
| sequence | | |
| Signal Peptide 6 | MFVFLVLLPLVSSQC | 154 |

### 2. 5′ Cap

A structural feature of mRNAs is cap structure at five-prime end (5′). Natural eukaryotic mRNA comprises a 7-methylguanosine cap linked to the mRNA via a 5′ to 5′-triphosphate bridge resulting in cap0 structure (m7GpppN). In most eukaryotic mRNA and some viral mRNA, further modifications can occur at the 2′-hydroxy-group (2′-OH) (e.g., the 2′-hydroxyl group may be methylated to form 2′-O-Me) of the first and subsequent nucleotides producing “cap1” and “cap2” five-prime ends, respectively). Diamond, et al., (2014) Cytokine & growth Factor Reviews, 25:543-550 reported that cap0-mRNA cannot be translated as efficiently as cap1-mRNA in which the role of 2′-O-Me in the penultimate position at the mRNA 5′ end is determinant. Lack of the 2′-O-met has been shown to trigger innate immunity and activate IFN response. Daffis, et al. (2010) Nature, 468:452-456; and Zust et al. (2011) Nature Immunology, 12:137-143.

RNA capping is well researched and is described, e.g., in Decroly E et al. (2012) Nature Reviews 10: 51-65; and in Ramanathan A. et al., (2016) Nucleic Acids Res; 44(16): 7511-7526, the entire contents of each of which is hereby incorporated by reference. For example, in some embodiments, a 5′-cap structure which may be suitable in the context of the present invention is a cap0 (methylation of the first nucleobase, e.g. m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (“anti-reverse cap analogue”), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

The term “5′-cap” as used herein refers to a structure found on the 5′-end of an RNA, e.g., mRNA, and generally includes a guanosine nucleotide connected to an RNA, e.g., mRNA, via a 5′- to 5′-triphosphate linkage (also referred to as Gppp or G(5′)ppp(5′)). In some embodiments, a guanosine nucleoside included in a 5′ cap may be modified, for example, by methylation at one or more positions (e.g., at the 7-position) on a base (guanine), and/or by methylation at one or more positions of a ribose. In some embodiments, a guanosine nucleoside included in a 5′ cap comprises a 3′0 methylation at a ribose (3′OMeG). In some embodiments, a guanosine nucleoside included in a 5′ cap comprises methylation at the 7-position of guanine (m7G). In some embodiments, a guanosine nucleoside included in a 5′ cap comprises methylation at the 7-position of guanine and a 3′ O methylation at a ribose (m7(3′OMeG)). It will be understood that the notation used in the above paragraph, e.g., “(m₂^{7,3′-O})G” or “m7(3′OMeG)”, applies to other structures described herein.

In some embodiments, providing an RNA with a 5′-cap disclosed herein may be achieved by in vitro transcription, in which a 5′-cap is co-transcriptionally expressed into an RNA strand, or may be attached to an RNA post-transcriptionally using capping enzymes. In some embodiments, co-transcriptional capping with a cap disclosed improves the capping efficiency of an RNA compared to co-transcriptional capping with an appropriate reference comparator. In some embodiments, improving capping efficiency can increase a translation efficiency and/or translation rate of an RNA, and/or increase expression of an encoded polypeptide. In some embodiments, alterations to polynucleotides generates a non-hydrolyzable cap structure which can, for example, prevent decapping and increase RNA half-life.

In some embodiments, T7 RNA polymerase prefers G as the initial site. Accordingly, in some such embodiments, the present disclosure provides caps (e.g., trinucleotide and tetranucleotide caps described herein) wherein the 3′end of the trinucleotide (e.g., N₂) or tetranucleotide cap (e.g., N₃) is G.

In some embodiments, it will be appreciated that all compounds or structures (e.g., 5′ caps) provided herein encompass the free base or salt form (e.g., an Na⁺ salt) comprising a suitable counterion (e.g., Na⁺). Compounds or structures (e.g., 5′ caps) depicted as a salt also encompass the free base and include suitable counterions (e.g., Na⁺).

In some embodiments, a utilized 5′ cap is a cap0, a cap1, or cap2 structure. See, e.g., FIG. 1 of Ramanathan A et al., and FIG. 1 of Decroly E et al., each of which is incorporated herein by reference in its entirety. See, e.g., FIG. 1 of Ramanathan A et al., and FIG. 1 of Decroly E et al., each of which is incorporated herein by reference in its entirety. In some embodiments, an RNA described herein comprises a cap1 structure. In some embodiments, an RNA described herein comprises a cap2.

In some embodiments, an RNA described herein comprises a cap0 structure. In some embodiments, a cap0 structure comprises a guanosine nucleoside methylated at the 7-position of guanine ((m⁷)G). In some embodiments, such a cap0 structure is connected to an RNA via a 5′- to 5′-triphosphate linkage and is also referred to herein as (m⁷)Gppp. In some embodiments, a cap0 structure comprises a guanosine nucleoside methylated at the 2′-position of the ribose of guanosine. In some embodiments, a cap0 structure comprises a guanosine nucleoside methylated at the 3′-position of the ribose of guanosine. In some embodiments, a guanosine nucleoside included in a 5′ cap comprises methylation at the 7-position of guanine and at the 2′-position of the ribose ((m₂^{7,2′-O})G). In some embodiments, a guanosine nucleoside included in a 5′ cap comprises methylation at the 7-position of guanine and at the 2′-position of the ribose ((m₂^{7,3′-O})G).

In some embodiments, a cap1 structure comprises a guanosine nucleoside methylated at the 7-position of guanine ((m⁷)G) and optionally methylated at the 2′ or 3′ position pf the ribose, and a 2′O methylated first nucleotide in an RNA ((m^{2′-O})N₁). In some embodiments, a cap1 structure comprises a guanosine nucleoside methylated at the 7-position of guanine ((m⁷)G) and the 3′ position of the ribose, and a 2′O methylated first nucleotide in an RNA ((m^{2′-O})N₁). In some embodiments, a cap1 structure is connected to an RNA via a 5′- to 5′-triphosphate linkage and is also referred to herein as, e.g., ((m⁷)Gppp(^{2′-O})N₁) or (m₂^{7,3′-O})Gppp(^{2′-O})N₁), wherein N₁ is as defined and described herein. In some embodiments, a cap1 structure comprises a second nucleotide, N₂, which is at position 2 and is chosen from A, G, C, or U, e.g., (m⁷)Gppp(^{2′-O})N₁pN₂ or (m₂^{7,3′-O})Gppp(^{2′-O})N₁pN₂, wherein each of N₁ and N₂ is as defined and described herein.

In some embodiments, a cap2 structure comprises a guanosine nucleoside methylated at the 7-position of guanine ((m⁷)G) and optionally methylated at the 2′ or 3′ position of the ribose, and a 2′O methylated first and second nucleotides in an RNA ((m^{2′-O})N₁p(m^{2′-O})N₂). In some embodiments, a cap2 structure comprises a guanosine nucleoside methylated at the 7-position of guanine ((m⁷)G) and the 3′ position of the ribose, and a 2′O methylated first and second nucleotide in an RNA. In some embodiments, a cap2 structure is connected to an RNA via a 5′- to 5′-triphosphate linkage and is also referred to herein as, e.g., ((m⁷)Gppp(^{2′-O})N₁p(^{2′-O})N₂) or (m₂^{7,3′-O})Gppp(^{2′-O})N₁p(^{2′-O})N₂), wherein each of N₁ and N₂ is as defined and described herein.

In some embodiments, the 5′ cap is a dinucleotide cap structure. In some embodiments, the 5′ cap is a dinucleotide cap structure comprising N₁, wherein N₁ is as defined and described herein. In some embodiments, the 5′ cap is a dinucleotide cap G*N₁, wherein N₁ is as defined above and herein, and:
G* comprises a structure of formula (I):

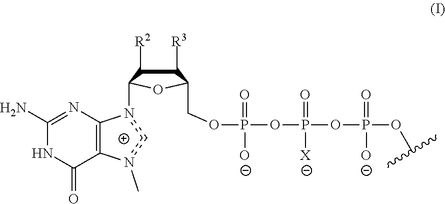

or a salt thereof,
wherein each R² and R³ is —OH or —OCH₃; and X is O or S.

In some embodiments, R² is —OH. In some embodiments, R² is —OCH₃. In some embodiments, R³ is —OH. In some embodiments, R³ is —OCH₃. In some embodiments, R² is —OH and R³ is —OH. In some embodiments, R² is —OH and R³ is —CH₃. In some embodiments, R² is —CH₃ and R³ is —OH. In some embodiments, R² is —CH₃ and R³ is —CH₃.

In some embodiments, X is O. In some embodiments, X is S.

In some embodiments, the 5′ cap is a dinucleotide cap0 structure (e.g., (m⁷)GpppN1, (m₂^{7,2′-O})GpppN1, (m₂^{7,2′-O})GpppN1, (m⁷)GppSpN₁, (m₂^{7,2′-O})GppSpN1, or (m₂^{7,3′-O})GppSpN₁), wherein N₁ is as defined and described herein. In some embodiments, the 5′ cap is a dinucleotide cap0 structure (e.g., (m⁷)GpppN₁, (m₂^{7,2′-O})GpppN₁, (m₂^{7,3′-O})GpppN₁, (m⁷)GppSpN₁, (m₂^{7,2′-O})GppSpN₁, or (m₂^{7,3′-O})GppSpN₁), wherein N₁ is G. In some embodiments, the 5′ cap is a dinucleotide cap0 structure (e.g., (m⁷)GpppN₁, (m₂^{7,2′-O})GpppN₁, (m₂^{7,2′-O})GpppN1, (m⁷)GppSpN₁, (m₂^{7,2′-O})GppSpN₁, or (m₂^{7,3′-O})GppSpN₁), wherein N₁ is A, U, or C. In some embodiments, the 5′ cap is a dinucleotide cap1 structure (e.g., (m⁷)Gppp(m^{2′-O})N₁, (m₂^{7,2′-O})Gppp(m^{2′-O})N₁, (m₂^{7,3′-O})Gppp(m^{2′-O})N₁, (m⁷)GppSp(m^{2′-O})N₁, (m₂^{7,2′-O})GppSp(m^{2′-O})N₁, or (m₂^{7,3′-O})GppSp(m^{2′-O})N₁), wherein N₁ is as defined and described herein. In some embodiments, the 5′ cap is selected from the group consisting of (m⁷)GpppG (“Ecap0”), (m⁷)Gppp(m^{2′-O})G (“Ecap1”), (m₂^{7,3′-O})GpppG (“ARCA” or “D1”), and (m₂^{7,2′-O})GppSpG (“beta-S-ARCA”). In some embodiments, the 5′ cap is (m⁷)GpppG (“Ecap0”), having a structure:

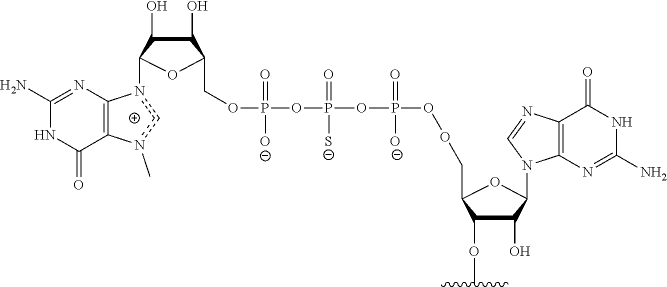

or a salt thereof.

In some embodiments, the 5′ cap is (m⁷)Gppp(m^{2′-O})G (“Ecap1”), having a structure:

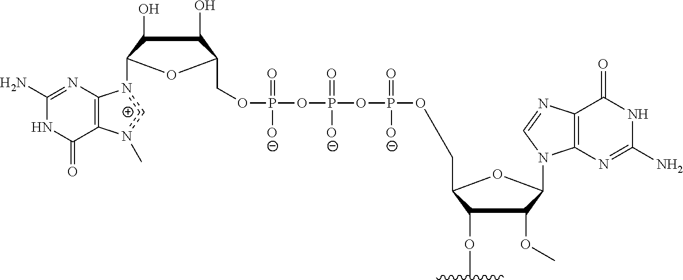

or a salt thereof.

In some embodiments, the 5′ cap is (m₂^{7,3′-O})GpppG (“ARCA” or “D1”), having a structure:

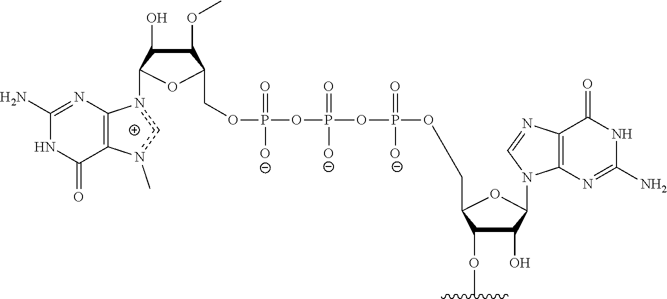

or a salt thereof.

In some embodiments, the 5′ cap is (m₂^{7,2′-O})GppSpG (“beta-S-ARCA”), having a structure:

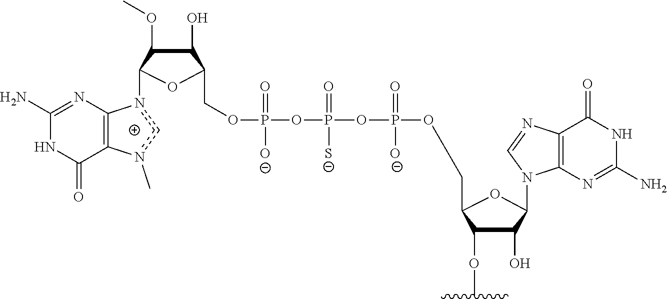

or a salt thereof.

In some embodiments, the 5′ cap is a trinucleotide cap structure. In some embodiments, the 5′ cap is a trinucleotide cap structure comprising N₁pN₂, wherein N₁ and N₂ are as defined and described herein. In some embodiments, the 5′ cap is a trinucleotide cap G*N₁pN₂, wherein N₁ and N₂ are as defined above and herein, and:
G* comprises a structure of formula (I):

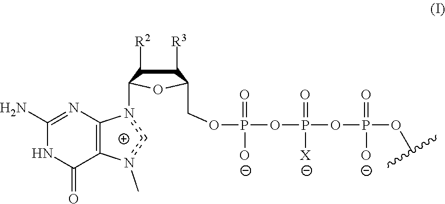

or a salt thereof, wherein R², R³, and X are as defined and described herein.

In some embodiments, the 5′ cap is a trinucleotide cap0 structure (e.g. (m⁷)GpppN₁pN₂, (m₂^{7,2′-O})GpppN₁pN₂, or (m₂^{7,2′-O})GpppN₁pN₂), wherein N₁ and N₂ are as defined and described herein). In some embodiments, the 5′ cap is a trinucleotide cap1 structure (e.g., (m⁷)Gppp(m^{2′-O})N₁pN₂, (m₂^{7,2′-O})Gppp(m^{2′-O})N₁pN₂, (m₂^{7,3′-O})Gppp(m^{2′-O})N₁pN₂), wherein N₁ and N₂ are as defined and described herein. In some embodiments, the 5′ cap is a trinucleotide cap2 structure (e.g., (m⁷)Gppp(m^{2′-O})N₁p(m^{2′-O})N₂, (m₂^{7,2′-O})Gppp(m^{2′-O})N₁p(m^{2′-O})N₂, (m₂^{7,3′-O})Gppp(m^{2′-O})N₁p(m^{2′-O})N₂), wherein N₁ and N₂ are as defined and described herein. In some embodiments, the 5′ cap is selected from the group consisting of (m₂^{7,2′-O})Gppp(m^{2′-O})ApG (“CleanCap AG 3′ OMe”, “CC413”), (m₂^{7,3′-O})Gppp(m^{2′-O})GpG (“CleanCap GG”), (m⁷)Gppp(m^{2′-O})ApG, (m⁷)Gppp(m^{2′-O})GpG, (m₂^{7,2′-O})Gppp(m₂^{6,2′-O})ApG, and (m⁷)Gppp(m^{2′-O})ApU. In some embodiments, the 5′ cap is selected from the group consisting of (m₂^{7,3′-O})Gppp(m^{2′-O})ApG (“CleanCap AG”, “CC413”), (m₂^{7,3′-O})Gppp(m^{2′-O})GpG (“CleanCap GG”), (m⁷)Gppp(m^{2′-O})ApG, and (m₂^{7,3′-O})Gppp(m₂^{6,2′-O})ApG, (m⁷)Gppp(m^{2′-O})ApU, and (m₂^{7,3′-O})Gppp(m^{2′-O})CpG.

In some embodiments, the 5′ cap is (m₂^{7,3′-O})Gppp(m^{2′-O})ApG (“CleanCap AG 3′ OMe”, “CC413”), having a structure:

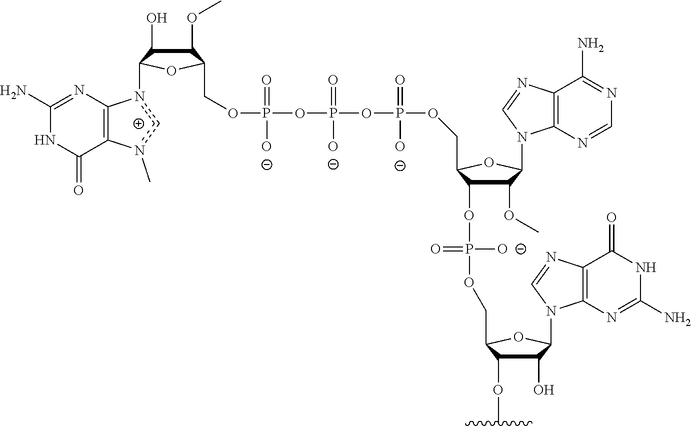

or a salt thereof.

In some embodiments, the 5′ cap is (m₂^{7,3′-O})Gppp(m^{2′-O})GpG (“CleanCap GG”), having a structure:

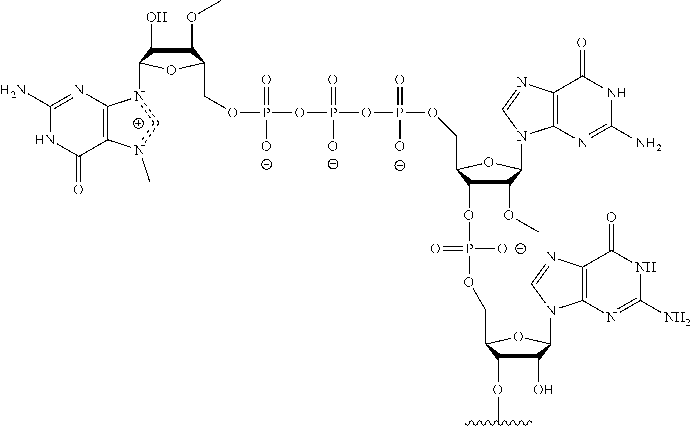

or a salt thereof.

In some embodiments, the 5′ cap is (m⁷)Gppp(m^{2′-O})ApG, having a structure:

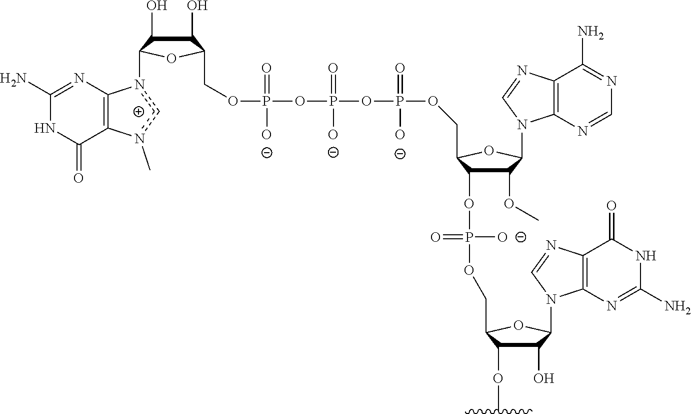

or a salt thereof.

In some embodiments, the 5′ cap is (m⁷)Gppp(m^{2′-O})GpG, having a structure:

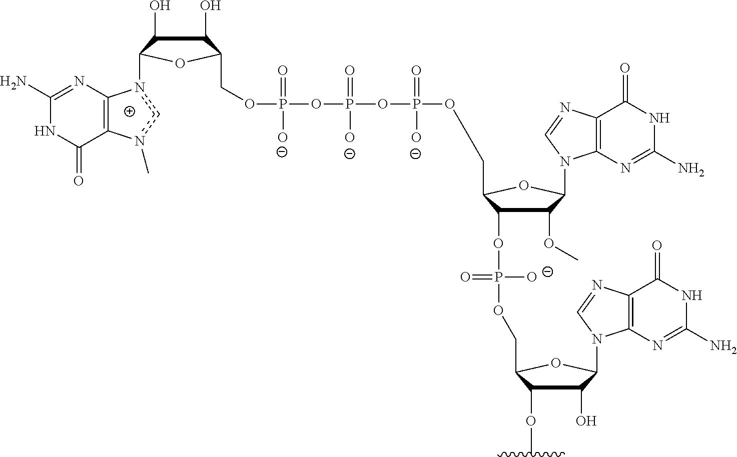

or a salt thereof.

In some embodiments, the 5′ cap is (m₂^{7,3′-O})Gppp(m₂^{6,2′-O})ApG, having a structure:

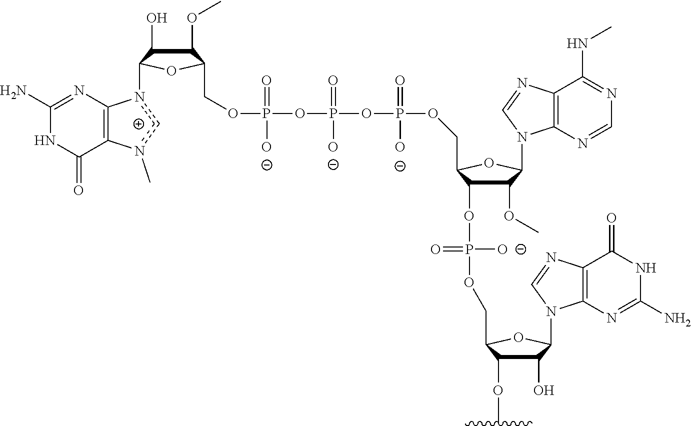

or a salt thereof.

In some embodiments, the 5′ cap is (m⁷)Gppp(m^{2′-O})ApU, having a structure:

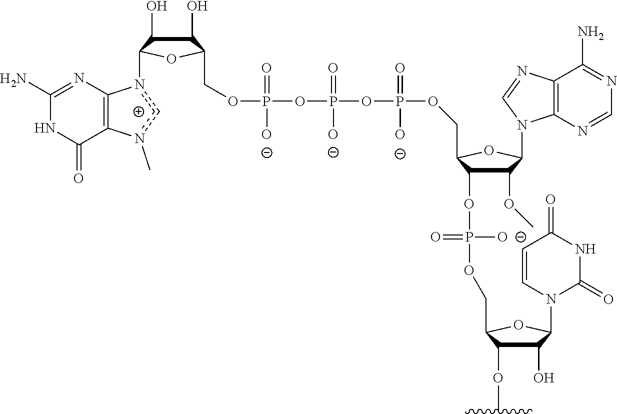

or a salt thereof.

In some embodiments, the 5′ cap is (m₂^{7,3′-O})Gppp(m^{2′-O})CpG, having a structure:

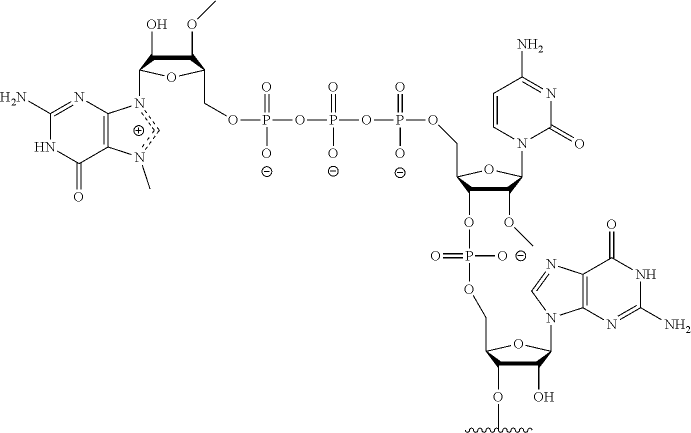

or a salt thereof.

In some embodiments, the 5′ cap is a tetranucleotide cap structure. In some embodiments, the 5′ cap is a tetranucleotide cap structure comprising N₁pN₂pN₃, wherein N₁, N₂, and N₃ are as defined and described herein. In some embodiments, the 5′ cap is a tetranucleotide cap G*N₁pN₂pN₃, wherein N₁, N₂, and N₃ are as defined above and herein, and:
G* comprises a structure of formula (I):

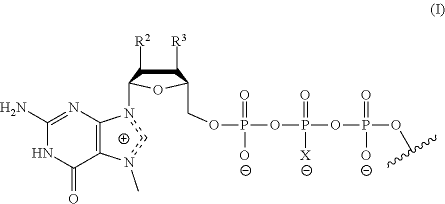

or a salt thereof, wherein R², R³, and X are as defined and described herein.

In some embodiments, the 5′ cap is a tetranucleotide cap0 structure (e.g. (m⁷)GpppN₁pN₂pN₃, (m₂^{7,2′-O})GpppN₁pN₂pN₃, or (m₂^{7,2′-O})GpppN₁N₂pN₃), wherein N₁, N₂, and N₃ are as defined and described herein). In some embodiments, the 5′ cap is a tetranucleotide Cap1 structure (e.g., (m⁷)Gppp(m^{2′-O})N₁pN₂pN₃, (m₂^{7,2′-O})Gppp(m^{2′-O})N₁pN₂pN₃, (m₂^{7,3′-O})Gppp(m^{2′-O})N₁pN₂N₃), wherein N₁, N₂, and N₃ are as defined and described herein. In some embodiments, the 5′ cap is a tetranucleotide Cap2 structure (e.g., (m⁷)Gppp(m^{2′-O})N₁p(m^{2′-O})N₂pN₃, (m₂^{7,2′-O})Gppp(m^{2′-O})N₁p(m^{2′-O})N₂pN₃, (m₂^{7,3′-O})Gppp(m^{2′—O)N}₁p(m^{2′-O})N₂pN₃), wherein N₁, N₂, and N₃ are as defined and described herein. In some embodiments, the 5′ cap is selected from the group consisting of (m₂^{7,3′-O})Gppp(m^{2′-O})Ap(m^{2′-O})GpG, (m₂^{7,3′-O})Gppp(m^{2′-O})Gp(m^{2′-O})GpC, (m⁷)Gppp(m^{2′-O})Ap(m^{2′-O})UpA, and (m⁷)Gppp(m^{2′-O})Ap(m^{2′-O})GpG.

In some embodiments, the 5′ cap is (m₂^{7,3′-O})Gppp(m^{2′-O})Ap(m^{2′-O})GpG, having a structure:

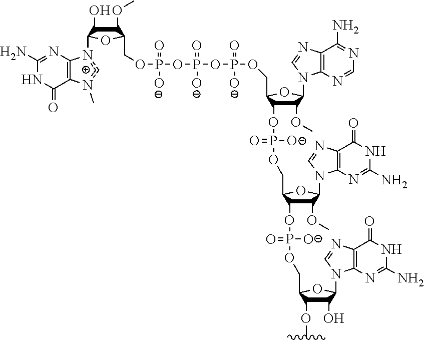

or a salt thereof.

In some embodiments, the 5′ cap is (m₂^{7,3′-O})Gpm^{2′-O}p(m^{2′-O})GpC, having a structure:

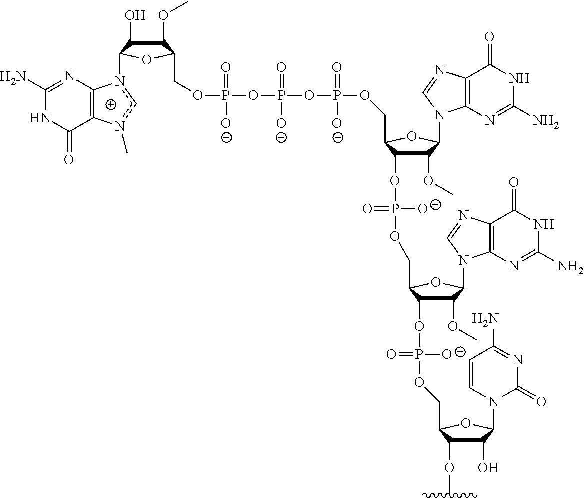

or a salt thereof.

In some embodiments, the 5′ cap is (m⁷)Gppp(m^{2′-O})Ap(m^{2′-O})UpA, having a structure:

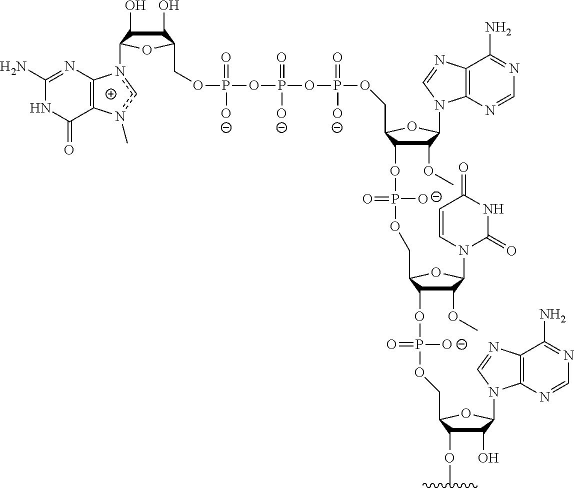

or a salt thereof.

In some embodiments, the 5′ cap is (m⁷)Gppp(m^{2′-O})Ap(m^{2′-O})GpG, having a structure:

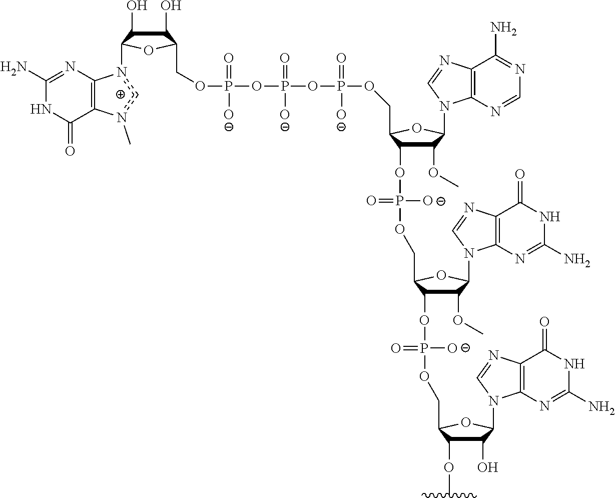

or a salt thereof.

In some embodiments, N₁ is A or an analog thereof. In some embodiments, N₁ is adenosine. In some embodiments, N₁ is modified adenosine. In some embodiments, N₁ is 6-methyladenosine. In some embodiments, N₁ is:

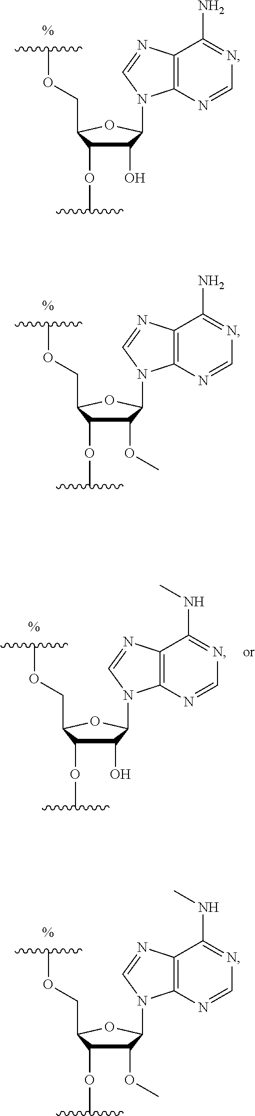

wherein % represents the point of attachment to G*.

In some embodiments, N₂ is U or an analog thereof. In some embodiments, N₂ is a modified U. In some embodiments, N₂ is 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (m⁵U), 1-taurinomethyl-pseudouridine, 5-taurinomethyl-2-thio-uridine(m⁵s²U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N₁-methyl-pseudouridine, 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

In some embodiments, N₂ is of formula (II):

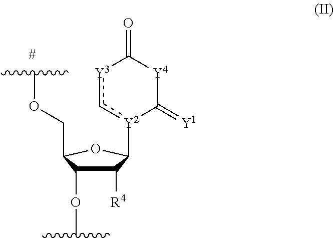

or a salt thereof, wherein:
each [symbol] is independently a single or double bond, as allowed by valency;
Y¹ is 0 or S;
Y² is N, C, or CH;
Y³ is N, NR^{a1}, CR^{a1}, or CHR^{a1};
Y⁴ is NR^{a2} or CHR^{a2};
each of R^{a1} or R^{a2} is independently hydrogen or C₁₋₆ aliphatic;
R⁴ is —OH or —OMe; and
#represents the point of attachment to p of Nip.

In some embodiments, Y¹ is O. In some embodiments, Y¹ is S.

In some embodiments, Y² is N. In some embodiments, Y² is C or CH. In some embodiments, Y² is C. In some embodiments, Y² is CH.

In some embodiments, Y³ is N or CR^{a1}. In some embodiments, Y³ is N. In some embodiments, Y³ is CR^{a1}. In some embodiments, Y³ is CH or C(CH₃). In some embodiments, Y³ is CH. In some embodiments, Y³ is C(CH₃). In some embodiments, Y³ is NR^{a1} or CHR^{a1}. In some embodiments, Y³ is NH or N(CH₃). In some embodiments, Y³ is NH, In some embodiments, Y³ is N(CH₃). In some embodiments, Y³ is CH₂ or CH(CH₃). In some embodiments, Y³ is CH₂. In some embodiments, Y³ is CH(CH₃).

In some embodiments, Y⁴ is NR^{a2}. In some embodiments, Y⁴ is NH or NCH₃. In some embodiments, Y⁴ is NH. In some embodiments, Y⁴ is NCH₃. In some embodiments, Y⁴ is CHR^{a2}. In some embodiments, Y⁴ is CH₂ or CH(CH₃). In some embodiments, Y⁴ is CH₂. In some embodiments, Y⁴ is CH(CH₃).

In some embodiments, R^{a1} is hydrogen. In some embodiments, R^{a1} is C₁₋₆ aliphatic. In some embodiments, R^{a1} is methyl, ethyl, n-propyl, or isopropyl. In some embodiments, R^{a1} is methyl.

In some embodiments, R^{a2} is hydrogen. In some embodiments, Ra2 is C₁₋₆ aliphatic. In some embodiments, R^{a2} is methyl, ethyl, n-propyl, or isopropyl. In some embodiments, R^{a2} is methyl.

In some embodiments, R⁴ is —OH. In some embodiments, R⁴ is —OMe.

In some embodiments, N² is of formula (IIa):

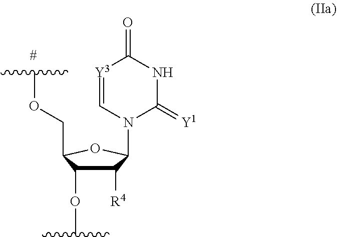

or a salt thereof, wherein each of Y¹, Y³, R⁴, and #is as defined above and described herein.

In some embodiments, N² is of formula (IIb):

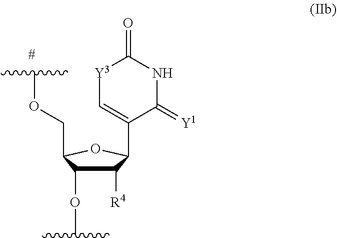

or a salt thereof, wherein each of Y¹, Y³, R⁴, and #is as defined above and described herein.

In some embodiments, N₂ is uridine, 1-methylpsuedouridine, 2-thio-uridine, or 5-methyluridine.

In some embodiments N₂ is:

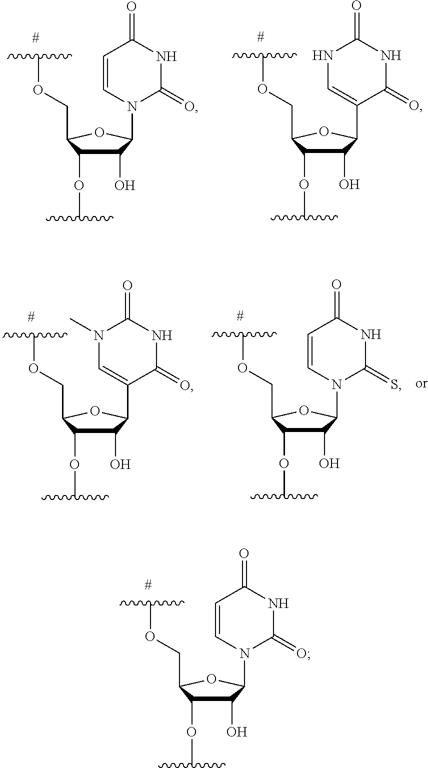

or a salt thereof, wherein #represents the point of attachment to p of Nip.

In some embodiments N₂ is:

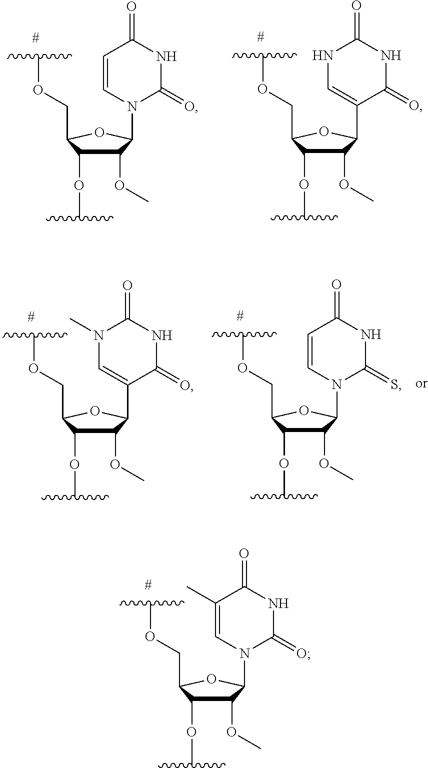

or a salt thereof, wherein #represents the point of attachment to p of Nip.

In some embodiments, p is —P(═O)(OH)—, or a salt thereof.

In some embodiments, the 5′ cap is (m^{7,2′-O})Gppp(m^{2′-O})A₁pU₂, (m^{7,3′-O})Gppp(m^{2′-O})A₁pU₂, (m^{7,2′-O})Gppp(m^{2′-O})A₁pΨ₂, (m^{7,3′-O})Gppp(m^{2′-O})Ap₂, (m^{7,2′-O})Gppp(m^{2′-O})A₁p(m¹)Ψ₂, (m^{7,3′-O})Gppp(m^{2′-O})A₁p(m¹)Ψ₂, (m^{7,2′-O})Gppp(m^{2′-O})A₁pS²U₂, (m^{7,3′-O})Gppp(m^{2′-O})A₁pS²U₂, (m^{7,2′-O})Gppp(m^{2′-O})A₁p(m⁵)U₂, or (m^{7,3′-O})Gppp(m^{2′-O})A₁p(m⁵)U₂.

In some embodiments, the 5′ cap is (m^{7,2′-O})Gppp(m^{6,2′-O})A₁pU₂, (m^{7,3′-O})Gppp(m^{6,2′-O})A₁pU₂, (m^{7,2′-O})Gppp(m^{6,2′-O})A₁pΨ₂, (m^{7,3′-O})Gppp(m^{6,2′-O})A₁pΨ₂, (m^{7,2′-O})Gppp(m^{6,2′-O})A₁p(m¹)Ψ₂, (m^{7,3′-O})Gppp(m^{6,2′-O})A₁p(m¹)Ψ₂, (m^{7,2′-O})Gppp(m^{6,2′-O})A₁pS²U₂, (m^{7,3′-O})Gppp(m^{6,2′-O})A₁pS²U₂, (m^{7,2′-O})Gppp(m^{6,2′-O})A₁p(m⁵)U₂, or (m^{7,3′-O})Gppp(m^{6,2′-O})A₁p(m⁵)U₂.

In some embodiments, the 5′ cap is (m^{7,2′-O})Gppp(m^{2′-O})A₁pU₂, having a structure:

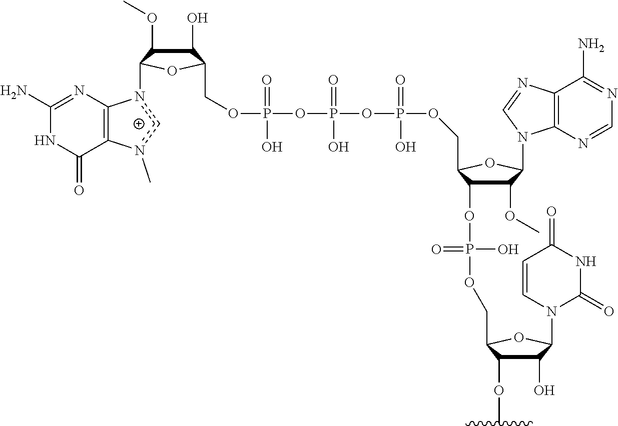

or a salt thereof.

In some embodiments, the 5′ cap is (m^{7,3′-O})Gppp(m^{2′-O})ApU₂,

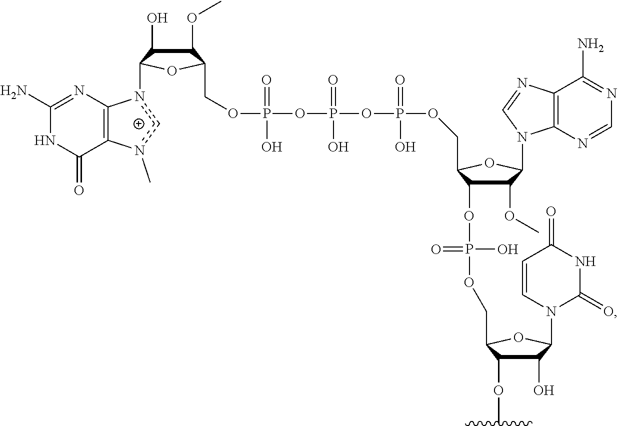

or a salt thereof.

In some embodiments, the 5′ cap is (m^{7,3′-O})Gppp(m^{2′-O})A₁pΨ₂,

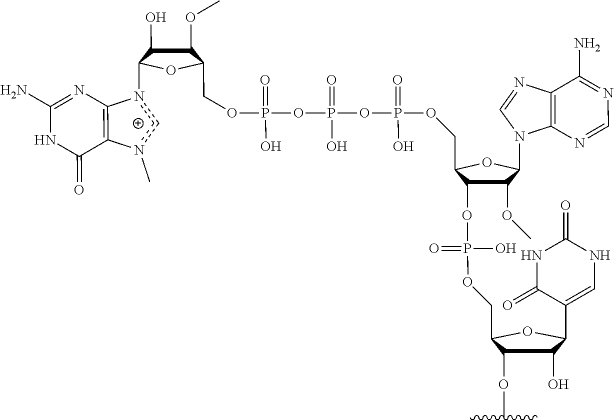

or a salt thereof.

In some embodiments, the 5′ cap is (m^{7,2′-O})Gppp(m^{2′-O})A₁pΨ₂,

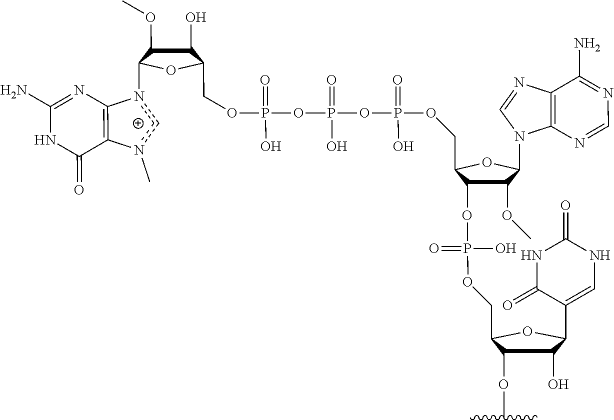

or a salt thereof.

In some embodiments, the 5′ cap is (m^{7,2′-O})Gppp(m^{2′-O})A₁p(m¹)Ψ₂,

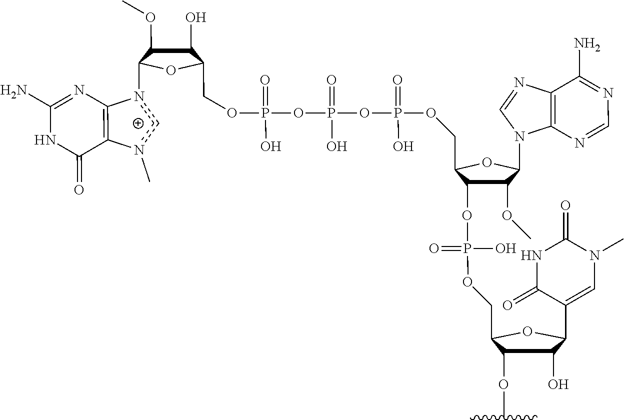

or a salt thereof.

In some embodiments, the 5′ cap is (m^{7,3′-O})ppp(m^{2′-O})A₁p(m¹)Ψ₂,

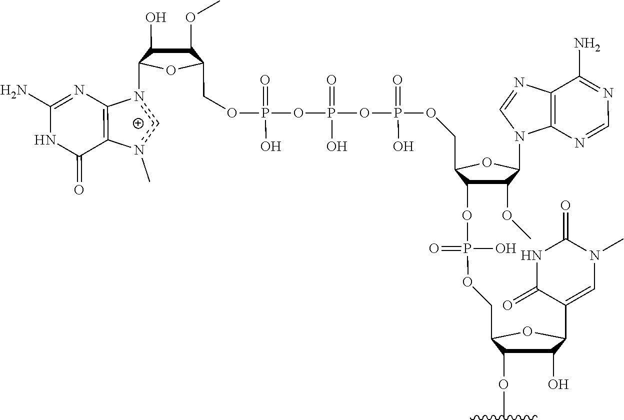

or a salt thereof.

In some embodiments, the 5′ cap is (m^{7,3′-O})Gppp(m^{2′-O})A₁pS²U₂,

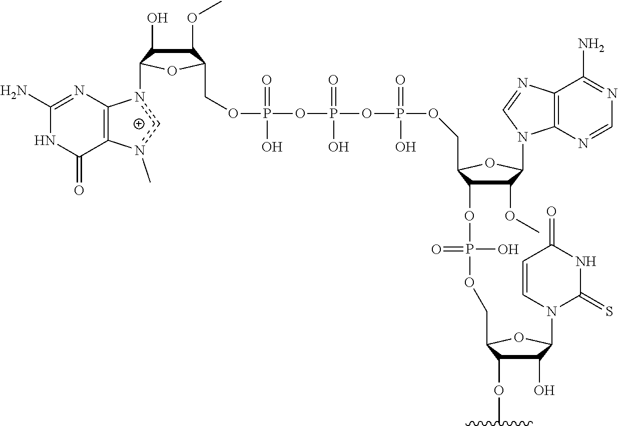

or a salt thereof.

In some embodiments, the 5′ cap is (m^{7,2′-O})Gppp(m^{2′-O})A₁pS²U₂,

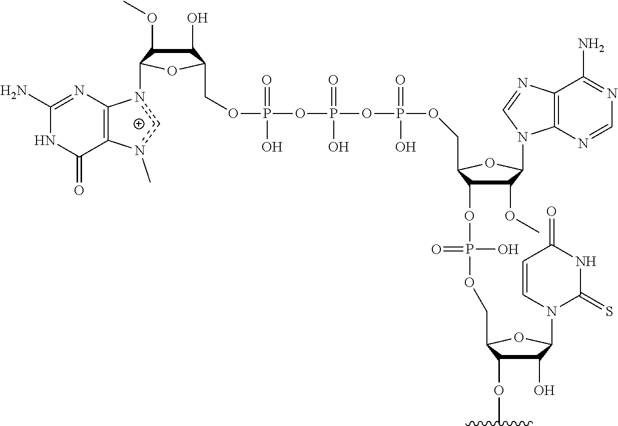

or a salt thereof.

In some embodiments, the 5′ cap is (m^{7,3′-O})Gppp(m^{2′-O})A₁p(m⁵)U₂,

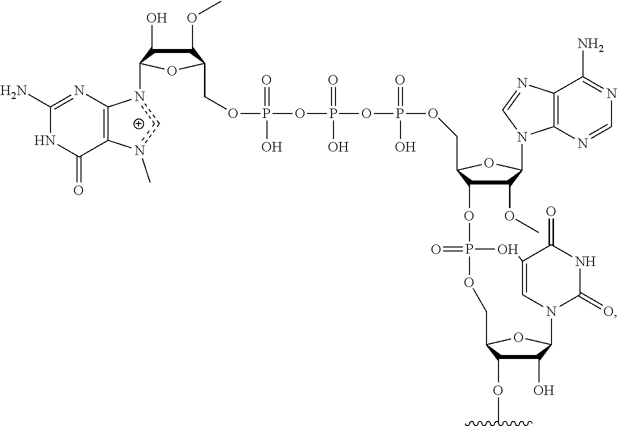

or a salt thereof.

In some embodiments, the 5′ cap is (m^{7,2′-O})Gppp(m^{2′-O})A₁p(m⁵)U₂,

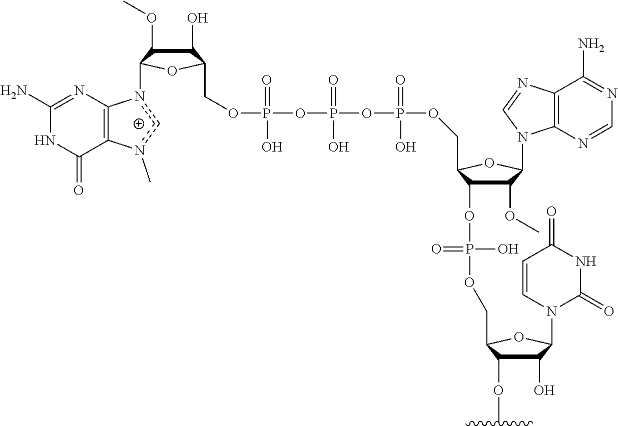

or a salt thereof.

In some embodiments, it will be appreciated that the disclosure of 5′ caps above and herein encompasses 5′ caps themselves or as part of a larger molecule (e.g., an RNA). For example, the structures drawn above encompass a 3′ ether linkage to the next nucleotide or as a free —OH.

In some embodiments, it will be appreciated that any of the structures above may exist in a salt form. For example, each of the phosphate groups may be deprotonated, e.g., —OP(═O)(O⁻)—, and be associated with an appropriate counterion.

### 3. Cap Proximal Sequences

In some embodiments, a 5′ UTR utilized in accordance with the present disclosure comprises a cap proximal sequence, e.g., as disclosed herein. In some embodiments, a cap proximal sequence comprises a sequence adjacent to a 5′ cap. In some embodiments, a cap proximal sequence comprises nucleotides in positions +1, +2, +3, +4, and/or +5 of an RNA polynucleotide.

In some embodiments, a cap structure comprises one or more polynucleotides of a cap proximal sequence. In some embodiments, a cap structure comprises an m⁷ Guanosine cap and nucleotide +1 (N₁) of an RNA polynucleotide. In some embodiments, a cap structure comprises an m⁷ Guanosine cap and nucleotide +2 (N₂) of an RNA polynucleotide. In some embodiments, a cap structure comprises an m⁷ Guanosine cap and nucleotides +1 and +2 (N₁ and N₂) of an RNA polynucleotide. In some embodiments, a cap structure comprises an m⁷ Guanosine cap and nucleotides +1, +2, and +3 (N₁, N₂, and N₃) of an RNA polynucleotide.

Those skilled in the art, reading the present disclosure, will appreciate that, in some embodiments, one or more residues of a cap proximal sequence (e.g., one or more of residues +1, +2, +3, +4, and/or +5) may be included in an RNA by virtue of having been included in a cap entity (e.g., a cap1 or cap2 structure, etc); alternatively, in some embodiments, at least some of the residues in a cap proximal sequence may be enzymatically added (e.g., by a polymerase such as a T7 polymerase). For example, in certain exemplified embodiments where a m₂^{7,3′-O}Gppp(m₁^{2′-O})ApG cap is utilized, +1 (i.e., N₁) and +2 (i.e. N₂) are the (m₁^{2′-O})A and G residues of the cap, and +3, +4, and +5 are added by polymerase (e.g., T7 polymerase).

In some embodiments, the 5′ cap is a dinucleotide cap structure, wherein the cap proximal sequence comprises N₁ of the 5′ cap, where N₁ is any nucleotide, e.g., A, C, G or U. In some embodiments, the 5′ cap is a trinucleotide cap structure (e.g., the trinucleotide cap structures described above and herein), wherein the cap proximal sequence comprises N₁ and N₂ of the 5′ cap, wherein N₁ and N₂ are independently any nucleotide, e.g., A, C, G or U. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁, N₂, and N₃ are any nucleotide, e.g., A, C, G or U.

In some embodiments, e.g., where the 5′ cap is a dinucleotide cap structure, a cap proximal sequence comprises N₁ of a the 5′ cap, and N₂, N₃, N₄ and N₅, wherein N₁ to N₅ correspond to positions +1, +2, +3, +4, and/or +5 of an RNA polynucleotide. In some embodiments, e.g., where the 5′ cap is a trinucleotide cap structure, a cap proximal sequence comprises N₁ and N₂ of a the 5′ cap, and N₃, N₄ and N₅, wherein N₁ to N₅ correspond to positions +1, +2, +3, +4, and/or +5 of an RNA polynucleotide. In some embodiments, e.g., where the 5′ cap is a tetranucleotide cap structure, a cap proximal sequence comprises N₁, N₂, and N₃ of a the 5′ cap, and N₄ and N₅, wherein N₁ to N₅ correspond to positions +1, +2, +3, +4, and/or +5 of an RNA polynucleotide.

In some embodiments, N₁ is A. In some embodiments, N₁ is C. In some embodiments, N₁ is G. In some embodiments, N₁ is U. In some embodiments, N₂ is A. In some embodiments, N₂ is C. In some embodiments, N₂ is G. In some embodiments, N₂ is U. In some embodiments, N₃ is A. In some embodiments, N₃ is C. In some embodiments, N₃ is G. In some embodiments, N₃ is U. In some embodiments, N₄ is A. In some embodiments, N₄ is C. In some embodiments, N₄ is G. In some embodiments, N₄ is U. In some embodiments, N₅ is A. In some embodiments, N₅ is C. In some embodiments, N₅ is G. In some embodiments, N₅ is U. It will be understood that, each of the embodiments described above and herein (e.g., for N₁ through N₅) may be taken singly or in combination and/or may be combined with other embodiments of variables described above and herein (e.g., 5′ caps).

### 4. 5′ UTR

In some embodiments, a nucleic acid (e.g., DNA, RNA) utilized in accordance with the present disclosure comprises a 5′-UTR. In some embodiments, a 5′-UTR may comprise a plurality of distinct sequence elements; in some embodiments, such plurality may be or comprise multiple copies of one or more particular sequence elements (e.g., as may be from a particular source or otherwise known as a functional or characteristic sequence element). In some embodiments a 5′ UTR comprises multiple different sequence elements.

The term “untranslated region” or “UTR” is commonly used in the art to refer to a region in a DNA molecule which is transcribed but is not translated into an amino acid sequence, or to the corresponding region in an RNA polynucleotide, such as an mRNA molecule. An untranslated region (UTR) can be present 5′ (upstream) of an open reading frame (5′-UTR) and/or 3′ (downstream) of an open reading frame (3′-UTR). As used herein, the terms “five prime untranslated region” or “5′ UTR” refer to a sequence of a polyribonucleotide between the 5′ end of the polyribonucleotide (e.g., a transcription start site) and a start codon of a coding region of the polyribonucleotide. In some embodiments, “5′ UTR” refers to a sequence of a polyribonucleotide that begins at the 5′ end of the polyribonucleotide (e.g., a transcription start site) and ends one nucleotide (nt) before a start codon (usually AUG) of a coding region of the polyribonucleotide, e.g., in its natural context. In some embodiments, a 5′ UTR comprises a Kozak sequence. A 5′-UTR is downstream of the 5′-cap (if present), e.g., directly adjacent to the 5′-cap. In some embodiments, a 5′ UTR disclosed herein comprises a cap proximal sequence, e.g., as defined and described herein. In some embodiments, a cap proximal sequence comprises a sequence adjacent to a 5′ cap.

Exemplary 5′ UTRs include a human alpha globin (hAg) 5′UTR or a fragment thereof, a TEV 5′ UTR or a fragment thereof, a HSP70 5′ UTR or a fragment thereof, or a c-Jun 5′ UTR or a fragment thereof.

In some embodiments, an RNA disclosed herein comprises a hAg 5′ UTR or a fragment thereof.

In some embodiments, an RNA disclosed herein comprises a 5′ UTR having at least 80%, at least 85%, at least 90%, at least 95%, at least 96%, at least 97%, at least 98%, or at least 99% identity to a 5′ UTR with the sequence according to SEQ ID NO: 155 (AGAATAAACTAGTATTCTTCTGGTCCCCACAGACTCAGAGAGAACCCGCCACC). In some embodiments, an RNA disclosed herein comprises a 5′ UTR provided in SEQ ID NO: 155.

In some embodiments, an RNA disclosed herein comprises a 5′ UTR having at least 80%, at least 85%, at least 90%, at least 95%, at least 96%, at least 97%, at least 98%, or at least 99% identity to a 5′ UTR with the sequence AACUAGUAUUCUUCUGGUCCCCACAGACUCAGAGAGAACCCGCCACC (SEQ ID NO: 272)(hAg-Kozak/5′UTR). In some embodiments, an RNA disclosed herein comprises a 5′ UTR provided in SEQ ID NO: 272.

### 5. PolyA Tail

In some embodiments, a polynucleotide (e.g., DNA, RNA) disclosed herein comprises a polyadenylate (polyA) sequence, e.g., as described herein. In some embodiments, a polyA sequence is situated downstream of a 3′-UTR, e.g., adjacent to a 3′-UTR.

As used herein, the term “poly(A) sequence” or “poly-A tail” refers to an uninterrupted or interrupted sequence of adenylate residues which is typically located at the 3′-end of an RNA polynucleotide. Poly(A) sequences are known to those of skill in the art and may follow the 3′-UTR in the RNAs described herein. An uninterrupted poly(A) sequence is characterized by consecutive adenylate residues. In nature, an uninterrupted poly(A) sequence is typical. In some embodiments, polynucleotides disclosed herein comprise an uninterrupted Poly(A) sequence. In some embodiments, polynucleotides disclosed herein comprise interrupted Poly(A) sequence. In some embodiments, RNAs disclosed herein can have a poly(A) sequence attached to the free 3′-end of the RNA by a template-independent RNA polymerase after transcription or a poly(A) sequence encoded by DNA and transcribed by a template-dependent RNA polymerase.

It has been demonstrated that a poly(A) sequence of about 120 A nucleotides has a beneficial influence on the levels of RNA in transfected eukaryotic cells, as well as on the levels of protein that is translated from an open reading frame that is present upstream (5′) of the poly(A) sequence (Holtkamp et al., 2006, Blood, vol. 108, pp. 4009-4017, which is herein incorporated by reference).

In some embodiments, a poly(A) sequence in accordance with the present disclosure is not limited to a particular length; in some embodiments, a poly(A) sequence is any length. In some embodiments, a poly(A) sequence comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly(A) sequence, typically at least 75%, at least 80%, at least 85%, at least 90%, at least 95%, at least 96%, at least 97%, at least 98%, or at least 99% by number of nucleotides in the poly(A) sequence are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly(A) sequence, i.e., 100% by number of nucleotides in the poly(A) sequence, are A nucleotides. The term “A nucleotide” or “A” refers to adenylate.

In some embodiments, a poly(A) sequence is attached during RNA transcription, e.g., during preparation of in vitro transcribed RNA, based on a DNA template comprising repeated dT nucleotides (deoxythymidylate) in the strand complementary to the coding strand. The DNA sequence encoding a poly(A) sequence (coding strand) is referred to as a poly(A) cassette.

In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in accordance with the present disclosure. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on a DNA level, constant propagation of plasmid DNA in E. coli and is still associated, on an RNA level, with the beneficial properties with respect to supporting RNA stability and translational efficiency is encompassed. In some embodiments, the poly(A) sequence contained in an RNA polynucleotide described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, no nucleotides other than A nucleotides flank a poly(A) sequence at its 3′-end, i.e., the poly(A) sequence is not masked or followed at its 3′-end by a nucleotide other than A.

In some embodiments, the poly(A) sequence may comprise at least 20, at least 30, at least 40, at least 80, or at least 100 and up to 500, up to 400, up to 300, up to 200, or up to 150 nucleotides. In some embodiments, the poly(A) sequence may essentially consist of at least 20, at least 30, at least 40, at least 80, or at least 100 and up to 500, up to 400, up to 300, up to 200, or up to 150 nucleotides. In some embodiments, the poly(A) sequence may consist of at least 20, at least 30, at least 40, at least 80, or at least 100 and up to 500, up to 400, up to 300, up to 200, or up to 150 nucleotides. In some embodiments, the poly(A) sequence comprises at least 100 nucleotides. In some embodiments, the poly(A) sequence comprises about 150 nucleotides. In some embodiments, the poly(A) sequence comprises about 120 nucleotides.

In some embodiments, a poly A tail comprises a specific number of adenosines, such as about 50 or more, about 60 or more, about 70 or more, about 80 or more, about 90 or more, about 100 or more, about 120, or about 150 or about 200. In some embodiments a poly A tail of a string construct may comprise 200 A residues or less. In some embodiments, a poly A tail of a string construct may comprise about 200 A residues. In some embodiments, a poly A tail of a string construct may comprise 180 A residues or less. In some embodiments, a poly A tail of a string construct may comprise about 180 A residues. In some embodiments, a poly A tail may comprise 150 residues or less.

In some embodiments, a poly(A) tail comprises a plurality of A residues interrupted by a linker. In some embodiments, a linker comprises the nucleotide sequence GCATATGAC (SEQ ID NO: 156).

In some embodiments, a polyribonucleotide of the present disclosure comprises a poly(A) sequence comprising the nucleotide sequence of SEQ ID NO: 268, or a nucleotide sequence having at least 99%, 98%, 97%, 96%, 95%, 90%, 85%, or 80% identity to the nucleotide sequence of SEQ ID NO: 268.

### 6. 3′ UTR

In some embodiments, a polyribonucleotide utilized in accordance with the present disclosure comprises a 3′-UTR. As used herein, the terms “three prime untranslated region,” “3′ untranslated region,” or “3′ UTR” refer to a sequence of an mRNA molecule that begins following a stop codon of a coding region of an open reading frame sequence. In some embodiments, the 3′ UTR begins immediately after a stop codon of a coding region of an open reading frame sequence, e.g., in its natural context. In other embodiments, the 3′ UTR does not begin immediately after the stop codon of the coding region of an open reading frame sequence, e.g., in its natural context. The term “3′-UTR” preferably does not include the poly(A) sequence. Thus, the 3′-UTR is upstream of the poly(A) sequence (if present), e.g. directly adjacent to the poly(A) sequence.

In some embodiments, an RNA disclosed herein comprises a 3′ UTR comprising an F element and/or an I element. In some embodiments, a 3′ UTR or a proximal sequence thereto comprises a restriction site. In some embodiments, a restriction site is a BamHI site. In some embodiments, a restriction site is a Xhol site.

In some embodiments, an RNA construct comprises an F element. In some embodiments, a F element sequence is a 3′-UTR of amino-terminal enhancer of split (AES).

In some embodiments, an RNA disclosed herein comprises a 3′ UTR.

In some embodiments, a 3′UTR is an FI element as described in WO2017/060314, which is herein incorporated by reference in its entirety.

### 7. Multimerization Elements

In some embodiments, a monkeypox antigen utilized as described herein includes a multimerization element (e.g., a heterologous multimerization element). In some embodiments, a heterologous multimerization element comprises a dimerization, trimerization or tetramerization element.

In some embodiments, a multimerization element is one described in WO2017/081082 (e.g., sequences of SEQ ID NOs: 1116-1167 of WO2017/081082, or fragments or variants thereof).

Exemplary trimerization and tetramerization elements include, but are not limited to, engineered leucine zippers, fibritin foldon domain from enterobacteria phage T4, GCN4p11, GCN4-p11, and p53.

In various embodiments, an antigen construct of the present disclosure includes, and/or a polyribonucleotide of the present disclosure encodes, an antigen operably linked with a multimizartion element such as a foldon domain. In various embodiments an antigen construct of the present disclosure includes, and/or a polyribonucleotide of the present disclosure encodes, an antigen operably linked with a foldon domain according to SEQ ID NO: 256 and/or encoded by a sequence according to SEQ ID NO: 257.

In some embodiments, a provided antigen is able to form a trimeric complex. For example, a utilized antigen may comprise a domain allowing formation of a multimeric complex, such as for example a trimeric complex of an amino acid sequence comprising a monkeypox antigen as described herein. In some embodiments, a domain allowing formation of a multimeric complex comprises a trimerization domain, for example, a trimerization domain as described herein.

In some embodiments, a monkeypox antigen can be modified by addition of a T4-fibritin-derived “foldon” trimerization domain, for example, to increase its immunogenicity.

### 8. Membrane Association Elements

In some embodiments, a monkeypox antigen as described herein includes a membrane association element (e.g., a heterologous membrane association element), such as a transmembrane domain.

A transmembrane domain can be N-terminal, C-terminal, or internal to an antigen. A coding sequence of a transmembrane element is typically placed in frame (i.e., in the same reading frame), 5′, 3′, or internal to coding sequences (e.g., monkeypox antigen coding sequences) with which it is to be linked.

In some embodiments, a transmembrane domain comprises or is a transmembrane domain of Hemagglutinin (HA) of Influenza virus, Env of HIV-1, equine infectious anaemia virus (EIAV), murine leukaemia virus (MLV), mouse mammary tumor virus, G protein of vesicular stomatitis virus (VSV), Rabies virus, or a seven transmembrane domain receptor.

In various embodiments, an antigen construct of the present disclosure includes, and/or a polyribonucleotide of the present disclosure encodes, an antigen operably linked with a transembrane domain. In various embodiments an antigen construct of the present disclosure includes, and/or a polyribonucleotide of the present disclosure encodes, an antigen operably linked with a HSV-1 gD transmembrane domain (TM) domain according to SEQ ID NO: 254 and/or encoded by a sequence according to SEQ ID NO: 255.

### B. RNA Formats

At least three distinct formats useful for RNA compositions (e.g., pharmaceutical compositions) have been developed, namely non-modified uridine containing mRNA (uRNA), nucleoside-modified mRNA (modRNA), and self-amplifying mRNA (saRNA). Each of these platforms displays unique features. In general, in all three formats, RNA is capped, contains open reading frames (ORFs) flanked by untranslated regions (UTR), and have a polyA-tail at the 3′ end. An ORF of an uRNA and modRNA vector encodes an antigen or fragment thereof. An saRNA has multiple ORFs.

In some embodiments, the RNA described herein may have modified nucleosides. In some embodiments, the RNA comprises a modified nucleoside in place of at least one (e.g. every) uridine.

The term “uracil,” as used herein, describes one of the nucleobases that can occur in the nucleic acid of RNA. The structure of uracil is:

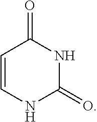

The term “uridine,” as used herein, describes one of the nucleosides that can occur in RNA. The structure of uridine is:

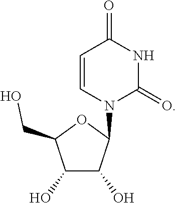 [SMILES: O=c1ccn([C@@H]2O[C@H](CO)[C@@H](O)[C@H]2O)c(=O)[nH]1]

UTP (uridine 5′-triphosphate) has the following structure:

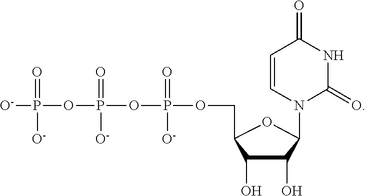

Pseudo-UTP (pseudouridine 5′-triphosphate) has the following structure:

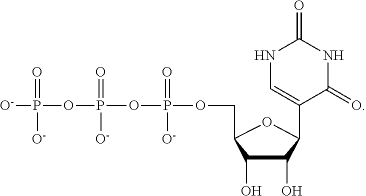

“Pseudouridine” is one example of a modified nucleoside that is an isomer of uridine, where the uracil is attached to the pentose ring via a carbon-carbon bond instead of a nitrogen-carbon glycosidic bond.

Another exemplary modified nucleoside is N₁-methyl-pseudouridine (mliP), which has the structure:

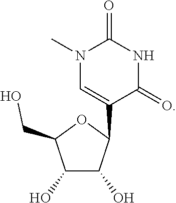 [SMILES: Cn1cc([C@@H]2O[C@H](CO)[C@@H](O)[C@H]2O)c(=O)[nH]c1=O]

N1-methyl-pseudo-UTP has the following structure:

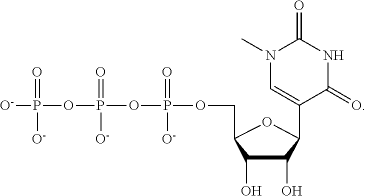

Another exemplary modified nucleoside is 5-methyl-uridine (m₅U), which has the structure:

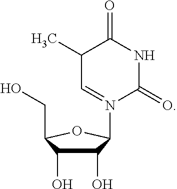

In some embodiments, one or more uridine in an RNA described herein is replaced by a modified nucleoside. In some embodiments, the modified nucleoside is a modified uridine.

In some embodiments, an RNA described herein comprises a modified nucleoside in place of at least one uridine. In some embodiments, an RNA described herein comprises a modified nucleoside in place of each uridine.

In some embodiments, the modified nucleoside is independently selected from pseudouridine (ψ), N1-methyl-pseudouridine (m1ψ), and 5-methyl-uridine (m5U). In some embodiments, the modified nucleoside comprises pseudouridine (ψ). In some embodiments, the modified nucleoside comprises N1-methyl-pseudouridine (m1ψ). In some embodiments, the modified nucleoside comprises 5-methyl-uridine (m5U). In some embodiments, RNA may comprise more than one type of modified nucleoside, and the modified nucleosides are independently selected from pseudouridine (ψ), N1-methyl-pseudouridine (m1ψ), and 5-methyl-uridine (m5U). In some embodiments, the modified nucleosides comprise pseudouridine (ψ) and N1-methyl-pseudouridine (m1ψ). In some embodiments, the modified nucleosides comprise pseudouridine (ψ) and 5-methyl-uridine (m5U). In some embodiments, the modified nucleosides comprise N1-methyl-pseudouridine (m1ψ) and 5-methyl-uridine (m5U). In some embodiments, the modified nucleosides comprise pseudouridine (ψ), N1-methyl-pseudouridine (m1ψ), and 5-methyl-uridine (m5U).

In some embodiments, the modified nucleoside replacing one or more, e.g., all, uridine in the RNA may be any one or more of 3-methyl-uridine (m3U), 5-methoxy-uridine (mo5U), 5-aza-uridine, 6-aza-uridine, 2-thio-5-aza-uridine, 2-thio-uridine (s²U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridine or 5-bromo-uridine), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 1-ethyl-pseudouridine, 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (m5U), 1-taurinomethyl-pseudouridine, 5-taurinomethyl-2-thio-uridine (m5s2U), 1-taurinomethyl-4-thio-pseudouridine), 5-methyl-2-thio-uridine (τm5s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl)pseudouridine (acp3 ψ), 5-(isopentenylaminomethyl)uridine (inm5U), 5-(isopentenylaminomethyl)-2-thio-uridine (inm5s²U), α-thio-uridine, 2′-O-methyl-uridine (Um), 5,2′-O-dimethyl-uridine (m5Um), 2′-O-methyl-pseudouridine (Wm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

In some embodiments, an RNA of the present disclosure comprises other modified nucleosides or comprises further modified nucleosides, e.g., modified cytidine. For example, in some embodiments, in an RNA of the present disclosure 5-methylcytidine is substituted partially or completely, preferably completely, for cytidine. In some embodiments, an RNA of the present disclosure comprises 5-methylcytidine and one or more selected from pseudouridine (yr), N1-methyl-pseudouridine (m1ψ), and 5-methyl-uridine (m5U). In some embodiments, an RNA of the present disclosure comprises 5-methylcytidine and N1-methyl-pseudouridine (m1ψ). In some embodiments, an RNA of the present disclosure comprises 5-methylcytidine in place of each cytidine and N1-methyl-pseudouridine (m1ψ) in place of each uridine.

In some embodiments of the present disclosure, an RNA is “replicon RNA” or simply a “replicon,” in particular “self-replicating RNA” or “self-amplifying RNA.” In one particularly preferred embodiment, the replicon or self-replicating RNA is derived from or comprises elements derived from a single-stranded (ss) RNA virus, in particular a positive-stranded ssRNA virus, such as an alphavirus. Alphaviruses are typical representatives of positive-stranded RNA viruses. Alphaviruses replicate in the cytoplasm of infected cells (for a review of the alphaviral life cycle see Jose et al., Future Microbiol., 2009, vol. 4, pp. 837-856, which is incorporated herein by reference in its entirety). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res., vol. 87 pp. 111-124). Following infection, i.e., at early stages of the viral life cycle, the (+) stranded genomic RNA directly acts like a messenger RNA for the translation of the open reading frame encoding the non-structural poly-protein (nsP1234).

Alphavirus-derived vectors have been proposed for delivery of foreign genetic information into target cells or target organisms. In simple approaches, a first ORF encodes an alphavirus-derived RNA-dependent RNA polymerase (replicase), which upon translation mediates self-amplification of the RNA. A second ORF encoding alphaviral structural proteins is replaced by an open reading frame encoding a protein of interest, e.g., an antigen or fragment thereof. Alphavirus-based trans-replication systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow for recognition and RNA synthesis by the alphaviral replicase.

Features of a non-modified uridine platform may include, for example, one or more of an intrinsic adjuvant effect, good tolerability, and improved safety. Features of modified uridine (e.g., pseudouridine) platform may include a reduced adjuvant effect, blunted immune innate immune sensor activating capacity, good tolerability and improved safety. Features of a self-amplifying platform may include, for example, long duration of protein expression, good tolerability and safety, and a higher likelihood for efficacy with a very low vaccine dose.

The present disclosure provides particular RNA constructs optimized, for example, for improved manufacturability, encapsulation, expression level (and/or timing), etc. Certain components are discussed below, and certain preferred embodiments are exemplified herein.

### C. Codon Optimization and GC Enrichment

As used herein, the term “codon-optimized” refers to alteration of codons in a coding region of a nucleic acid molecule (e.g., a polyribonucleotide) to reflect the typical codon usage of a host organism (e.g., a subject receiving a nucleic acid molecule (e.g., a polyribonucleotide)) without preferably altering the amino acid sequence encoded by the nucleic acid molecule. Within the context of the present disclosure, in some embodiments, coding regions are codon-optimized for optimal expression in a subject to be treated using the RNA molecules described herein. In some embodiments, codon-optimization may be performed such that codons for which frequently occurring tRNAs are available are inserted in place of “rare codons.” In some embodiments, codon-optimization may include increasing guanosine/cytosine (G/C) content of a coding region of RNA described herein as compared to the G/C content of the corresponding coding sequence of a wild type RNA, wherein the amino acid sequence encoded by the RNA is preferably not modified compared to the amino acid sequence.

In some embodiments, a coding sequence (also referred to as a “coding region”) is codon optimized for expression in the subject to whom a composition (e.g., a pharmaceutical composition) is to be administered (e.g., a human). Thus, in some embodiments, sequences in such a polynucleotide (e.g., a polyribonucleotide) may differ from wild type sequences encoding the relevant antigen or fragment or epitope thereof, even when the amino acid sequence of the antigen or fragment or epitope thereof is wild type.

In some embodiments, a coding sequence is codon optimized for expression in a relevant subject (e.g., a human), and even, in some cases, for expression in a particular cell or tissue.

Various species exhibit particular bias for certain codons of a particular amino acid. Without wishing to be bound by any one theory, codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell may generally be a reflection of the codons used most frequently in peptide synthesis. Accordingly, genes may be tailored for optimal gene expression in a given organism based on codon optimization. Codon usage tables are available, for example, at the “Codon Usage Database” available at www.kazusa.orjp/codon/and these tables may be adapted in a number of ways. Computer algorithms for codon optimizing a particular sequence for expression in a particular subject or its cells are also available, such as Gene Forge (Aptagen; Jacobus, PA), are also available.

In some embodiments, a polynucleotide (e.g., a polyribonucleotide) of the present disclosure is codon optimized, wherein the codons in the polynucleotide (e.g., the polyribonucleotide) are adapted to human codon usage (herein referred to as “human codon optimized polynucleotide”). Codons encoding the same amino acid occur at different frequencies in a subject, e.g., a human. Accordingly, in some embodiments, the coding sequence of a polynucleotide of the present disclosure is modified such that the frequency of the codons encoding the same amino acid corresponds to the naturally occurring frequency of that codon according to the human codon usage, e.g., as shown in Table 4. For example, in the case of the amino acid Ala, the wild type coding sequence is preferably adapted in a way that the codon “GCC” is used with a frequency of 0.40, the codon “GCT” is used with a frequency of 0.28, the codon “GCA” is used with a frequency of 0.22 and the codon “GCG” is used with 30 a frequency of 0.10 etc. (see Table 4). Accordingly, in some embodiments, such a procedure (as exemplified for Ala) is applied for each amino acid encoded by the coding sequence of a polynucleotide to obtain sequences adapted to human codon usage.

TABLE 4
Human codon usage table with frequencies
indicated for each amino acid.

| Amino acid | codon | frequency | Amino acid | codon | frequency |
|---|---|---|---|---|---|
| Ala | GCG | 0.10 | Pro | CCG | 0.11 |
| Ala | GCA | 0.22 | Pro | CCA | 0.27 |
| Ala | GCT | 0.28 | Pro | CCT | 0.29 |
| Ala | GCC* | 0.40 | Pro | CCC* | 0.33 |
| Cys | TGT | 0.42 | Gln | CAG* | 0.73 |
| Cys | TGC* | 0.58 | Gln | CAA | 0.27 |
| Asp | GAT | 0.44 | Arg | AGG | 0.22 |
| Asp | GAC* | 0.56 | Arg | AGA* | 0.21 |
| Glu | GAG* | 0.59 | Arg | CGG | 0.19 |
| Glu | GAA | 0.41 | Arg | CGA | 0.10 |
| Phe | TTT | 0.43 | Arg | CGT | 0.09 |
| Phe | TTC* | 0.57 | Arg | CGC | 0.19 |
| Gly | GGG | 0.23 | Ser | AGT | 0.14 |
| Gly | GGA | 0.26 | Ser | AGC* | 0.25 |
| Gly | GGT | 0.18 | Ser | TCG | 0.06 |
| Gly | GGC* | 0.33 | Ser | TCA | 0.15 |
| His | CAT | 0.41 | Ser | TCT | 0.18 |
| His | CAC* | 0.59 | Ser | TCC | 0.23 |
| Ile | ATA | 0.14 | Thr | ACG | 0.12 |
| Ile | ATT | 0.35 | Thr | ACA | 0.27 |
| Ile | ATC* | 0.52 | Thr | ACT | 0.23 |
| Lys | AAG* | 0.60 | Thr | ACC* | 0.38 |
| Lys | AAA | 0.40 | Val | GTG* | 0.48 |
| Leu | TTG | 0.12 | Val | GTA | 0.10 |
| Leu | TTA | 0.06 | Val | GTT | 0.17 |
| Leu | CTG* | 0.43 | Val | GTC | 0.25 |
| Leu | CTA | 0.07 | Trp | TGG* | 1 |
| Leu | CTT | 0.12 | Tyr | TAT | 0 42 |
| Leu | CTC | 0.20 | Tyr | TAC* | 0.58 |
| Met | ATG* | 1 | Stop | TGA* | 0 61 |
| Asn | AAT | 0.44 | Stop | TAG | 0.17 |
| Asn | AAC* | 0.56 | Stop | TAA | 0.22 |

Certain strategies for codon optimization and/or G/C enrichment for human expression are described in WO2002/098443, which is incorporated by reference herein in its entirety. In some embodiments, a coding sequence may be optimized using a multiparametric optimization strategy. In some embodiments, optimization parameters may include parameters that influence protein expression, which can be, for example, impacted on a transcription level, an mRNA level, and/or a translational level. In some embodiments, exemplary optimization parameters include, but are not limited to transcription-level parameters (including, e.g., GC content, consensus splice sites, cryptic splice sites, SD sequences, TATA boxes, termination signals, artificial recombination sites, and combinations thereof); mRNA-level parameters (including, e.g., RNA instability motifs, ribosomal entry sites, repetitive sequences, and combinations thereof); translation-level parameters (including, e.g., codon usage, premature poly(A) sites, ribosomal entry sites, secondary structures, and combinations thereof); or combinations thereof. In some embodiments, a coding sequence may be optimized by a GeneOptimizer algorithm as described in Fath et al. “Multiparameter RNA and Codon Optimization: A Standardized Tool to Assess and Enhance Autologous Mammalian Gene Expression” PLoS ONE 6(3): e17596; Rabb et al., “The GeneOptimizer Algorithm: using a sliding window approach to cope with the vast sequence space in multiparameter DNA sequence optimization” Systems and Synthetic Biology (2010) 4:215-225; and Graft et al. “Codon-optimized genes that enable increased heterologous expression in mammalian cells and elicit efficient immune responses in mice after vaccination of naked DNA” Methods Mol Med (2004) 94:197-210, the entire content of each of which is incorporated herein for the purposes described herein. In some embodiments, a coding sequence may be optimized by Eurofins' adaption and optimization algorithm “GENEius” as described in Eurofins' Application Notes: Eurofins' adaption and optimization software “GENEius” in comparison to other optimization algorithms, the entire content of which is incorporated by reference for the purposes described herein.

In some embodiments, a coding sequence utilized in accordance with the present disclosure has G/C content of which increased compared to a wild type coding sequence

Without wishing to be bound by any particular theory, it is proposed that GC enrichment may improve translation of a payload sequence. Typically, sequences having an increased G (guanosine)/C (cytidine) content are more stable than sequences having an increased A (adenosine)/U (uridine) content. In respect to the fact that several codons code for one and the same amino acid (so-called degeneration of the genetic code), the most favorable codons for the stability can be determined (so-called alternative codon usage). Depending on the amino acid to be encoded by a polyribonucelotide, there are various possibilities for modification of the ribonucleic acid sequence, compared to its wild type sequence. In particular, codons which contain A and/or U nucleosides can be modified by substituting these codons by other codons, which code for the same amino acids but contain no A and/or U or contain a lower content of A and/or U nucleosides.

In some embodiments, G/C content of a coding region of a polyribonucleotide described herein is increased by at least 1%, at least 2%, at least 3%, at least 4%, at least 5%, at least 6%, or even more compared to the G/C content of the coding region prior to codon optimization, e.g., of the wild type RNA. In some embodiments, G/C content of a coding region of a polyribonucleotide described herein is decreased by at least 1%, at least 2%, at least 3%, at least 4%, at least 5%, at least 6%, or even more compared to the G/C content of the coding region prior to codon optimization, e.g., of the wild type RNA.

In some embodiments, stability and translation efficiency of an polyribonucleotide may incorporate one or more elements established to contribute to stability and/or translation efficiency of the polyribonucleotide; exemplary such elements are described, for example, in PCT/EP2006/009448 incorporated herein by reference. In some embodiments, to increase expression of a polyribonucleotide used according to the present disclosure, a polyribonucleotide may be modified within the coding region, i.e., the sequence encoding the expressed peptide or protein, without altering the sequence of the expressed peptide or protein, for example so as to increase the GC-content to increase mRNA stability and/or to perform a codon optimization and, thus, enhance translation in cells.

### D. Exemplary Polyribonucleotide Sequences

The present disclosure includes certain exemplary antigen constructs and polyribonucleotides useful, e.g., in vaccination against orthopoxvirus (e.g. monkeypox), that encode and/or express one or more monkeypox antigens. In some embodiments, a polyribonucleotide, as described herein has one of the following structures:

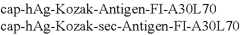

where cap refers to a 5′ cap as described above; hAg-Kozak refers to a 5′ UTR human alpha-globin; see refers to a secretion signal; Antigen refers to a nucleotide sequence comprising a sequence that encodes a monkeypox antigen described herein; FI refers to a 3′-UTR as described above, and A30L70 refers to a polyA sequence. In some embodiments, hAg 5′ UTR comprises a nucleotide sequence of SEQ ID NO: 155. In some embodiments, A30L70 comprises 30 adenine nucleotides followed by 70 adenine nucleotides, wherein the 30 adenine nucleotides and 70 adenine nucleotides are separated by a linker sequence.

### 1. Exemplary B Cell Antigen Polyribonucleotide Sequences

The present disclosure includes certain exemplary antigen constructs and polyribonucleotides useful, e.g., in vaccination against orthopox virus (e.g., monkeypox), that encode and/or express one or more antigens according to Table 1 or fragments thereof. In some embodiments, a polyribonucleotide of the present disclosure encodes a wildtype antigen sequence. In some embodiments, a polyribonucleotide of the present disclosure encodes an antigen polypeptide operably linked to an N-terminal viral signal peptide. Without wishing to be bound by any particular theory, inclusion of a viral signal peptide can be useful, e.g., because monkeypox antigens do not naturally include conventional secretion signal peptides and/or because inclusion of the viral signal peptide may allow for enhanced surface expression of the operably linked antigen on vaccinated cells.

Exemplary antigens of the present disclosure (e.g., A29L, A28L, H3L, and B6R or fragments thereof) can further include substitution of unpaired cysteine residues present in corresponding reference sequences. Without wishing to be bound by any particular scientific theory, the present disclosure includes that such cysteines, if left unpaired, carry a high risk of causing protein misfolding and/or aggregation and that this risk is mitigated by alanine substitutions. Exemplary substitutions can include positions C71A and/or C72A of A29L, C140A of B6R, and/or C86A and/or C90A of H3L.

For the avoidance of doubt, the present disclosure includes exemplary polypeptide sequences and polyribonucleic acid sequences as described herein and/or as set forth in sequence identification numbers of the present disclosure, as well as sequences having at least 75%, at least 80%, at least 85%, at least 90%, at least 95%, at least 96%, at least 97%, at least 98%, or at least 99% identity thereto.

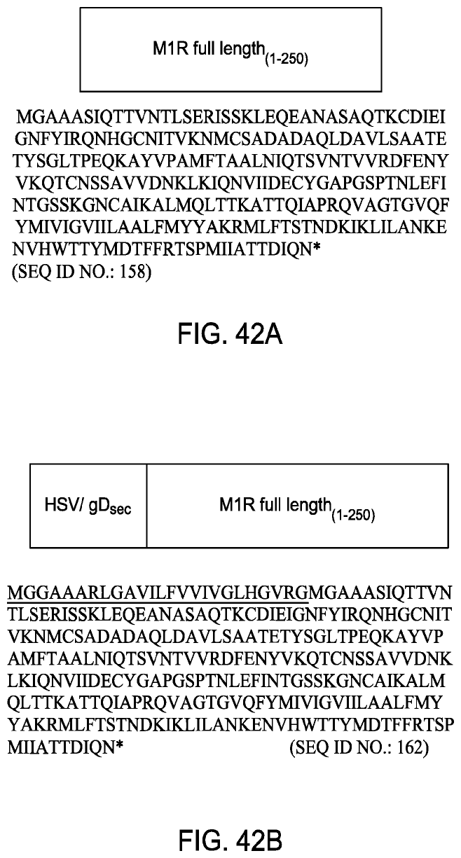

In some embodiments, a polyribonucleotide of the present disclosure encodes a wildtype M1R polypeptide. In some embodiments, a wildtype M1R polypeptide has or includes a sequence according to SEQ ID NO: 158 (see, e.g., FIG. 42A) and/or the polyribonucleotide has or includes a sequence according to SEQ ID NO: 159. In some embodiments, a polyribonucleotide of the present disclosure encodes a wildtype M1R polypeptide operably linked with a signal peptide such as an HSV/gD secretory sequence (HSV/gD_{sec}) according to SEQ ID NO: 160, optionally encoded by a sequence according to SEQ ID NO: 161. In some embodiments, a wildtype M1R polypeptide operably linked with an HSV/gD secretory sequence has or includes a sequence according to SEQ ID NO: 162 (see, e.g., FIG. 42B) and/or the encoding polyribonucleotide has or includes a sequence according to SEQ ID NO: 162.

In some embodiments, a polyribonucleotide of the present disclosure encodes a wildtype M1R polypeptide. In some embodiments, a wildtype M1R polypeptide has or includes a sequence according to SEQ ID NO: 258 and/or the polyribonucleotide encoding the wildtype M1R polypeptide has or includes a sequence according to SEQ ID NO: 259. In some embodiments, a polyribonucleotide of the present disclosure encodes a wildtype M1R polypeptide operably linked with a signal peptide such as an HSV/gD secretory sequence (HSV/gD_{sec}) according to SEQ ID NO: 160, optionally encoded by a sequence according to SEQ ID NO: 161.

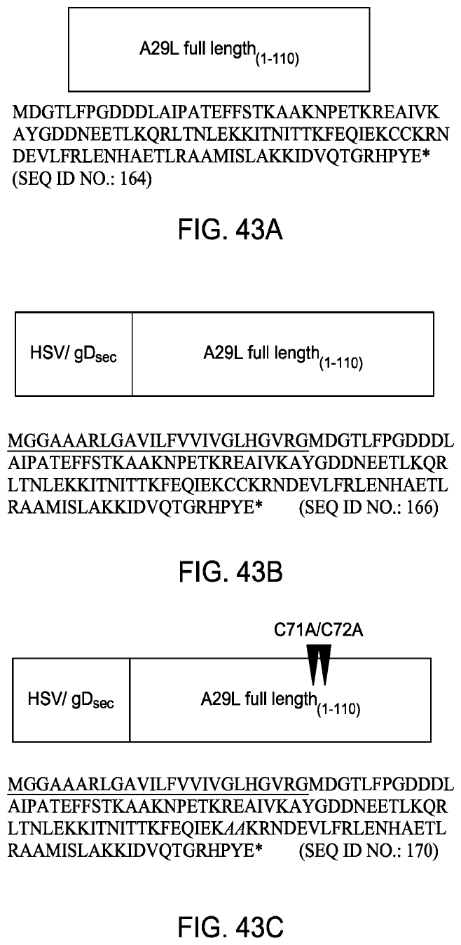

In some embodiments, a polyribonucleotide of the present disclosure encodes a wildtype A29L polypeptide. In some embodiments, a wildtype A29L polypeptide has or includes a sequence according to SEQ ID NO: 164 (see, e.g., FIG. 43A) and/or the polyribonucleotide encoding said wildtype A29L polypeptide has or includes a sequence according to SEQ ID NO: 165. In some embodiments, a polyribonucleotide of the present disclosure encodes a wildtype A29L polypeptide operably linked with a signal peptide such as an HSV/gD secretory sequence (HSV/gD_{sec}) according to SEQ ID NO: 160, optionally encoded by a sequence according to SEQ ID NO: 161. In some embodiments, a wildtype A29L polypeptide operably linked with an HSV/gD secretory sequence has or includes a sequence according to SEQ ID NO: 166 (see, e.g., FIG. 43B) and/or the encoding polyribonucleotide has or includes a sequence according to SEQ ID NO: 167.

In some embodiments, a polyribonucleotide of the present disclosure encodes an A29L polypeptide that includes a substitution of CC to AA at positions 71 and 72 corresponding to SEQ ID NO: 168 (substitutions C71A and C72A as compared to a corresponding reference sequence). In some embodiments, an A29L polypeptide including C71A and C72A substitutions has or includes a sequence according to SEQ ID NO: 168 and/or the polyribonucleotide encoding said A29L polypeptide has or includes a sequence according to SEQ ID NO: 169. In some embodiments, a polyribonucleotide of the present disclosure encodes an A29L polypeptide including C71A and C72A substitutions operably linked with a signal peptide such as an HSV/gD secretory sequence (HSV/gD_{sec}) according to SEQ ID NO: 160, optionally encoded by a sequence according to SEQ ID NO: 161. In some embodiments, an A29L polypeptide including C71A and C72A substitutions operably linked with an HSV/gD secretory sequence has or includes a sequence according to SEQ ID NO: 170 (see, e.g., FIG. 43C) and/or the encoding polyribonucleotide has or includes a sequence according to SEQ ID NO: 171.

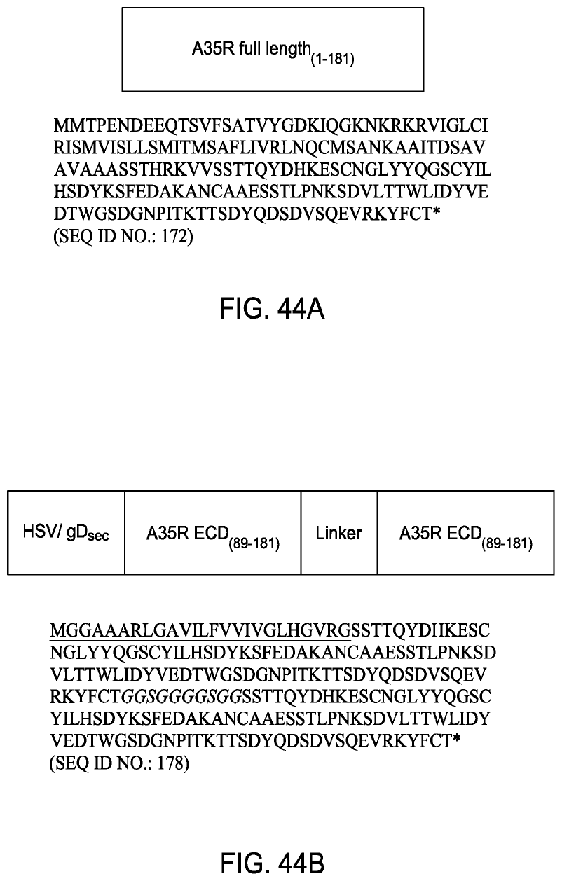

In some embodiments, a polyribonucleotide of the present disclosure encodes a soluble form of the ectodomain of A35R. Without wishing to be bound by any particular scientific theory, a soluble form of the ecotodomain of A35R would function independently of membrane insertion, reducing the potential need or requirement for membrane insertion of an A35R antigen or fragment thereof. The A35R ectodomain disclosed herein can have or include a sequence according to SEQ ID NO: 174. The boundaries of the ectodomain were informed by two X-ray crystallography studies defining the structure of this region of the protein. The present inventors selected amino acids 89-181 for use in this design at least in part because the selected amino acids span the resolved region of the protein elucidated by these studies. A35R forms a dimer that is partially dependent on a disulfide bond between residues not included in this ectodomain. To compensate for the absence of this disulfide, the present inventors engineered a construct that includes two copies of the ectodomain sufficient to form the dimer with a linker between them. Without wishing to be limited or bound by any particular scientific theory, linker size (10 amino acids) was selected based on the measured distance between the C-terminus of one ectodomain and the N-terminus of its binding partner in the crystal structure of the ectodomain, while those of skill in the art will appreciate that the linker could be larger or smaller, and any linker disclosed herein could be used. (See, e.g., FIG. 44B)

In some embodiments, a polyribonucleotide of the present disclosure encodes a wildtype A35R polypeptide. In some embodiments, a wildtype A35R polypeptide has or includes a sequence according to SEQ ID NO: 172 (see, e.g., FIG. 44A) and/or the polyribonucleotide encoding said A35R polypeptide has or includes a sequence according to SEQ ID NO: 173. In some embodiments, a polyribonucleotide of the present disclosure encodes a wildtype A35R ectodomain (ECD) fragment that has or includes a sequence according to SEQ ID NO: 174 and/or the encoding polyribonucleotide has or includes a sequence according to SEQ ID NO: 175. In some embodiments, a polyribonucleotide of the present disclosure encodes a first wildtype A35R ECD fragment and a second wildtype A35R ECD fragment, where the first wildtype A35R ECD fragment has or includes a sequence according to SEQ ID NO: 174 and/or is encoded by a sequence according to SEQ ID NO: 175 and the second wildtype A35R ECD fragment has or includes a sequence according to SEQ ID NO: 174 and/or is encoded by a sequence according to SEQ ID NO: 175, optionally wherein the first wildtype A35R fragment and the second wildtype A35R fragment are operably linked via linker (e.g., a linker according to SEQ ID NO: 176 and/or encoded by SEQ ID NO: 177). In some embodiments, a polyribonucleotide of the present disclosure encodes a first wildtype A35R fragment and a second wildtype A35R fragment, where the first wildtype A35R fragment and the second wildtype A35R fragment are operably linked by a linker, and where the first and second wildtype A35R fragments are operably linked with a signal peptide such as an HSV/gD secretory sequence (HSV/gD_{sec}) according to SEQ ID NO: 160, optionally encoded by a sequence according to SEQ ID NO: 161. In certain such embodiments, a polypeptide encoded by a polyribonucleotide has or includes a sequence according to SEQ ID NO: 178 (see, e.g., FIG. 44B) and/or the encoding polyribonucleotide has or includes a sequence according to SEQ ID NO: 179.

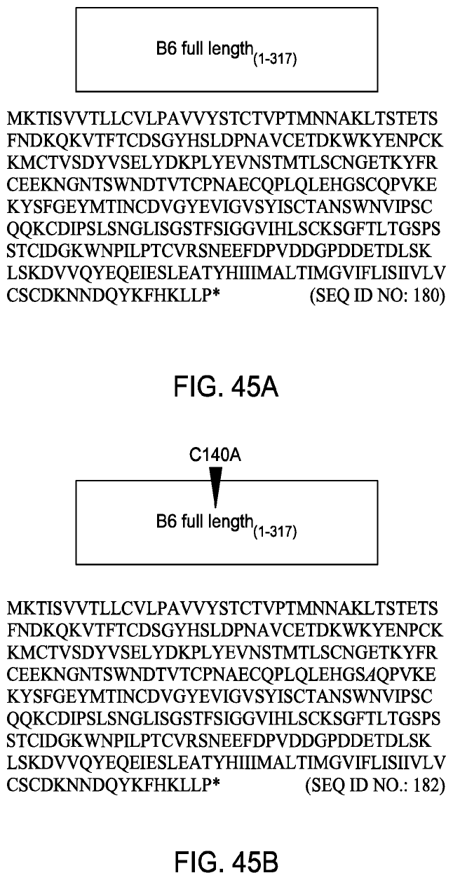

In some embodiments, a polyribonucleotide of the present disclosure encodes a wildtype B6R polypeptide. In some embodiments, a wildtype B6R polypeptide has or includes a sequence according to SEQ ID NO: 180 (see, e.g., FIG. 45A) and/or the polyribonucleotide encoding said B6R polypeptide has or includes a sequence according to SEQ ID NO: 181. In some embodiments, a polyribonucleotide of the present disclosure encodes an B6R polypeptide that includes a substitution of C to A at position 140 corresponding to SEQ ID NO: 182 (substitution C140A as compared to a corresponding reference sequence). In some embodiments, a B6R polypeptide including a C140A substitution has or includes a sequence according to SEQ ID NO: 182 (see, e.g., FIG. 45B) and/or the encoding polyribonucleotide has or includes a sequence according to SEQ ID NO: 183.

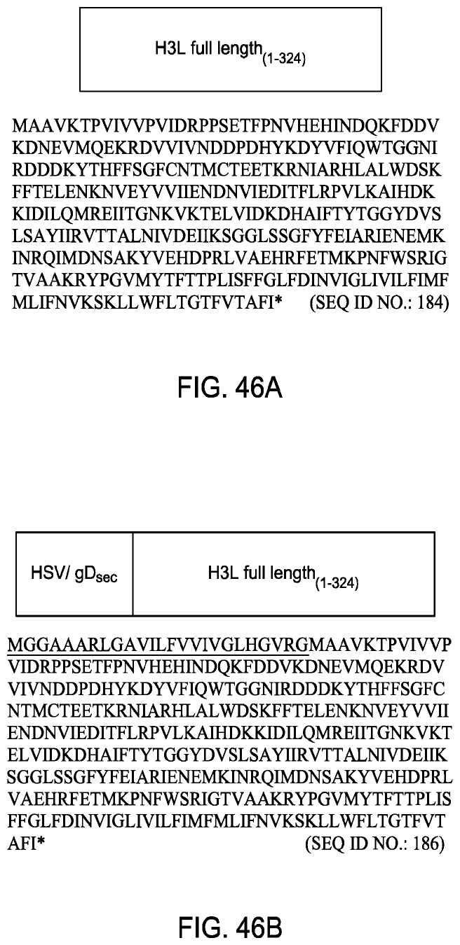

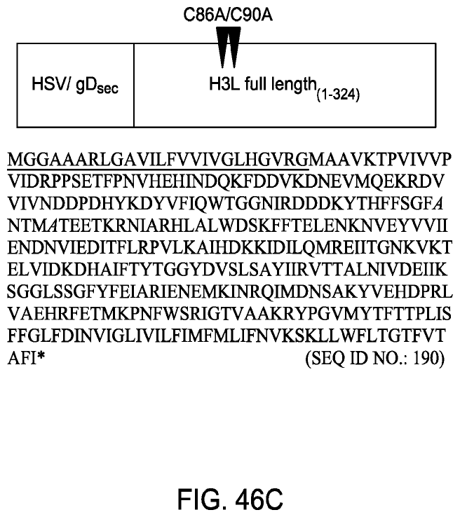

In some embodiments, a polyribonucleotide of the present disclosure encodes a wildtype H3L polypeptide. In some embodiments, a wildtype H3L polypeptide has or includes a sequence according to SEQ ID NO: 184 (see, e.g., FIG. 46A) and/or the polyribonucleotide encoding said H3L polypeptide has or includes a sequence according to SEQ ID NO: 185. In some embodiments, a polyribonucleotide of the present disclosure encodes a wildtype H3L polypeptide operably linked with a signal peptide such as an HSV/gD secretory sequence (HSV/gD_{sec}) according to SEQ ID NO: 160, optionally encoded by a sequence according to SEQ ID NO: 161. In some embodiments, a wildtype H3L polypeptide operably linked with an HSV/gD secretory sequence has or includes a sequence according to SEQ ID NO: 186 (see, e.g., FIG. 46B) and/or the encoding polyribonucleotide has or includes a sequence according to SEQ ID NO: 187.

In some embodiments, a polyribonucleotide of the present disclosure encodes an H3L polypeptide that includes a substitution of C to A at positions 86 and 90 corresponding to SEQ ID NO: 188 (substitutions C86A and C90A as compared to a corresponding reference sequence). In some embodiments, an H3L polypeptide including C86A and C90A substitutions has or includes a sequence according to SEQ ID NO: 188 and/or the polyribonucleotide encoding said H3L polypeptide has or includes a sequence according to SEQ ID NO: 189. In some embodiments, a polyribonucleotide of the present disclosure encodes an H3L polypeptide including C86A and C90A substitutions operably linked with a signal peptide such as an HSV/gD secretory sequence (HSV/gD_{sec}) according to SEQ ID NO: 160, optionally encoded by a sequence according to SEQ ID NO: 161. In some embodiments, an H3L polypeptide including C86A and C90A substitutions operably linked with an HSV/gD secretory sequence has or includes a sequence according to SEQ ID NO: 190 (see, e.g., FIG. 46C) and/or the encoding polyribonucleotide has or includes a sequence according to SEQ ID NO: 191.

In some embodiments, a polyribonucleotide of the present disclosure encodes a wildtype H3L polypeptide. In some embodiments, a wildtype H3L polypeptide has or includes a sequence according to SEQ ID NO: 260 and/or the polyribonucleotide encoding said H3L polypeptide has or includes a sequence according to SEQ ID NO: 261. In some embodiments, a polyribonucleotide of the present disclosure encodes a wildtype H3L polypeptide operably linked with a signal peptide such as an HSV/gD secretory sequence (HSV/gD_{sec}) according to SEQ ID NO: 160, optionally encoded by a sequence according to SEQ ID NO: 161.

In some embodiments, a polyribonucleotide of the present disclosure encodes an H3L polypeptide that includes a substitution of C to A at positions 86 and 90 corresponding to SEQ ID NO: 262 (substitutions C86A and C90A as compared to a corresponding reference sequence). In some embodiments, an H3L polypeptide including C86A and C90A substitutions has or includes a sequence according to SEQ ID NO: 262 and/or the polyribonucleotide encoding said H3L polypeptide has or includes a sequence according to SEQ ID NO: 263. In some embodiments, a polyribonucleotide of the present disclosure encodes an H3L polypeptide including C86A and C90A substitutions operably linked with a signal peptide such as an HSV/gD secretory sequence (HSV/gD_{sec}) according to SEQ ID NO: 160, optionally encoded by a sequence according to SEQ ID NO: 161.

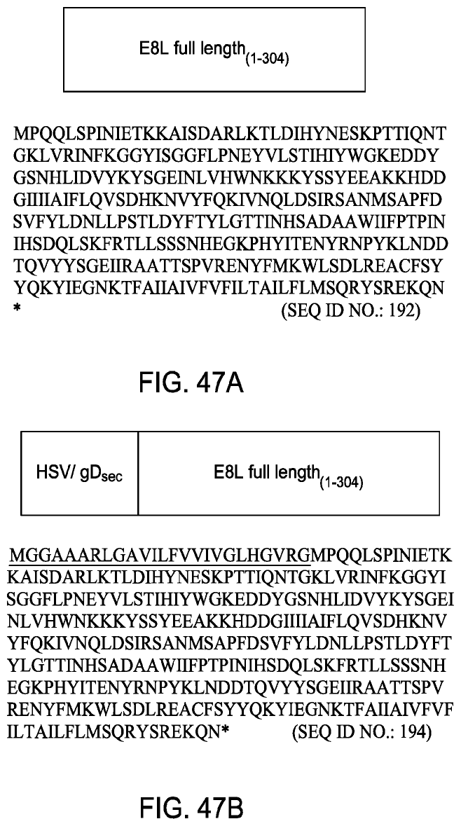

In some embodiments, a polyribonucleotide of the present disclosure encodes a wildtype E8L polypeptide. In some embodiments, a wildtype E8L polypeptide has or includes a sequence according to SEQ ID NO: 192 (see, e.g., FIG. 47A) and/or the polyribonucleotide encoding said E8L polypeptide has or includes a sequence according to SEQ ID NO: 193. In some embodiments, a polyribonucleotide of the present disclosure encodes a wildtype E8L polypeptide operably linked with a signal peptide such as an HSV/gD secretory sequence (HSV/gD_{sec}) according to SEQ ID NO: 160, optionally encoded by a sequence according to SEQ ID NO: 161. In some embodiments, a wildtype E8L polypeptide operably linked with an HSV/gD secretory sequence has or includes a sequence according to SEQ ID NO: 194 (see, e.g., FIG. 47B) and/or the encoding polyribonucleotide has or includes a sequence according to SEQ ID NO: 195.

In some embodiments, a polyribonucleotide of the present disclosure encodes a wildtype E8L polypeptide. In some embodiments, a wildtype E8L polypeptide has or includes a sequence according to SEQ ID NO: 264 and/or the polyribonucleotide encoding said E8L polypeptide has or includes a sequence according to SEQ ID NO: 265. In some embodiments, a polyribonucleotide of the present disclosure encodes a wildtype E8L polypeptide operably linked with a signal peptide such as an HSV/gD secretory sequence (HSV/gD_{sec}) according to SEQ ID NO: 160, optionally encoded by a sequence according to SEQ ID NO: 161.

In some embodiments, a polyribonucleotide of the present disclosure encodes a wildtype E8L polypeptide. In some embodiments, a wildtype E8L polypeptide has or includes a sequence according to SEQ ID NO: 266 and/or the polyribonucleotide encoding said E8L polypeptide has or includes a sequence according to SEQ ID NO: 267. In some embodiments, a polyribonucleotide of the present disclosure encodes a wildtype E8L polypeptide operably linked with a signal peptide such as an HSV/gD secretory sequence (HSV/gD_{sec}) according to SEQ ID NO: 160, optionally encoded by a sequence according to SEQ ID NO: 161.

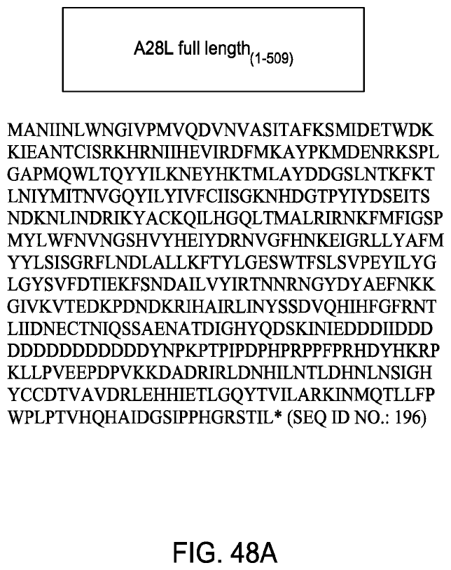

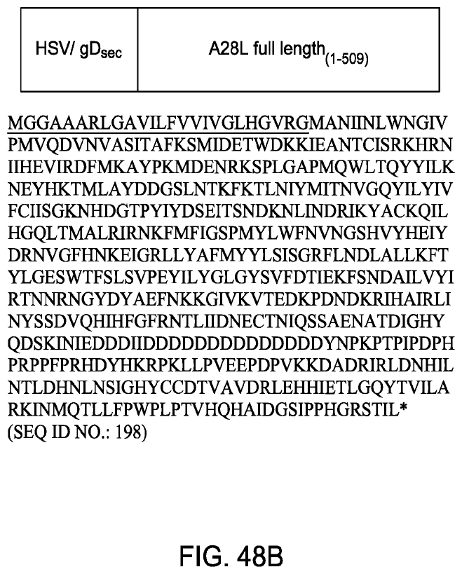

In some embodiments, a polyribonucleotide of the present disclosure encodes a wildtype A28L polypeptide. In some embodiments, a wildtype A28L polypeptide has or includes a sequence according to SEQ ID NO: 196 (see, e.g., FIG. 48A) and/or the polyribonucleotide encoding said A28L polypeptide has or includes a sequence according to SEQ ID NO: 197. In some embodiments, a polyribonucleotide of the present disclosure encodes a wildtype A28L polypeptide operably linked with a signal peptide such as an HSV/gD secretory sequence (HSV/gD_{sec}) according to SEQ ID NO: 160, optionally encoded by a sequence according to SEQ ID NO: 161. In some embodiments, a wildtype A28L polypeptide operably linked with an HSV/gD secretory sequence has or includes a sequence according to SEQ ID NO: 198 (see, e.g., FIG. 48B) and/or the encoding polyribonucleotide has or includes a sequence according to SEQ ID NO: 199.

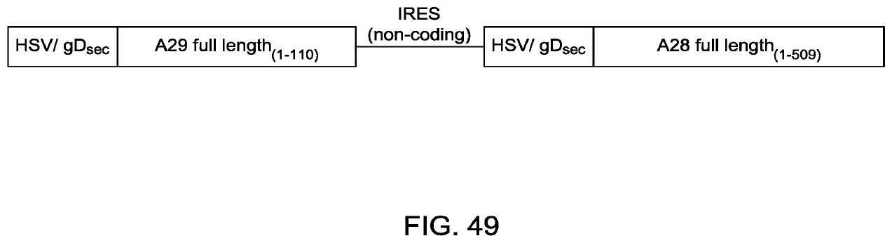

In some embodiments, a polyribonucleotide of the present disclosure is a bicistronic sequence that encodes A29L and A28L. A29L and A28L form heterodimers that assemble into large macromolecular complexes on the surface of monkeypox virions. This interaction is stabilized by a pair of disulfide bonds between A29L and A28L formed by sequential cysteine residues in each protein (A29L: C71, C72, A28L: C441, C442). The present inventors designed constructs to produce a native configuration of these proteins (the large complex of heterodimers). The present disclosure includes a bicistronic construct that ensures A29L and A28L will be co-expressed within one cell. The bicistronic polyribonucleotide encodes a full A29L ORF and a full A28L ORF, with an internal ribosome entry site between them. (See, e.g., FIG. 49)

In some embodiments, the present disclosure includes a bicstronic polyribonucleotide that (i) encodes an A29L antigen according to SEQ ID NO: 171 and/or has or includes a sequence according to SEQ ID NO: 200, and (ii) encodes an A28L antigen according to SEQ ID NO: 196 and/or has or includes a sequence according to SEQ ID NO: 197. In some embodiments, a bicistronic polyribonucleotide of the present disclosure encodes an A29L polypeptide operably linked with a signal peptide such as an HSV/gD secretory sequence (HSV/gD_{sec}) according to SEQ ID NO: 160, optionally encoded by a sequence according to SEQ ID NO: 161. In some embodiments, a bicistronic polyribonucleotide of the present disclosure encodes an A28L polypeptide operably linked with a signal peptide such as an HSV/gD secretory sequence (HSV/gD_{sec}) according to SEQ ID NO: 160, optionally encoded by a sequence according to SEQ ID NO: 161. In some embodiments, polyribonucleotide sequences encoding A29L and A28L are separated by an IRES. In some embodiments, a bicstronic polyribonucleotide of the present disclosure encodes an A29L polypeptide operably linked with a signal peptide such as an HSV/gD secretory sequence (HSV/gD_{sec}) according to SEQ ID NO: 173 and/or encoded by SEQ ID NO: 201, and an A28L polypeptide operably linked with a signal peptide such as an HSV/gD secretory sequence (HSV/gD_{sec}) according to SEQ ID NO: 198 and/or encoded by SEQ ID NO: 199, separated by an IRES (e.g., SEQ ID NO: 202) as encoded by the sequence according to SEQ ID NO: 203. (See, e.g., FIG. 49).

### 2. Exemplary T Cell Antigen Polyribonucleotide Sequences

The present disclosure includes certain exemplary antigen constructs and polyribonucleotides useful, e.g., in vaccination against orthopox virus (e.g., monkeypox), that encode one or more antigens according to Table 2 or fragments thereof. As disclosed herein, polyribonucleotides encoding T cell antigens and/or antigens of Table 2 can include fragments of antigens that are or include a T cell epitope. In various embodiments disclosed herein, a polyribonucleotide encoding a T cell antigen or fragment thereof and/or an antigen of Table 2 or fragment thereof can encode a plurality or “string” of such antigens or fragments thereof. Antigens of a string can be, for example, associated via linkers. Polyribonucleotides encoding T cell antigens can also include a signal peptide such as an HSV-1 gD secretion signal (e.g., according to SEQ ID NO: 218). Polyribonucleotides encoding T cell antigens can also encode an MITD domain (e.g., according to SEQ ID NO: 219). Without wishing to be bound by any particular scientific theory, an MITD domain can promote shuttling of an expressed polyprotein string to the proteasome of a host cell to enhance epitope presentation and T cell responses.

The present disclosure includes, among other things, exemplary fragments of T cell antigens that the present inventors have determined include T cell epitopes and/or can be encoded by polyribonucleotides of the present disclosure, e.g., polyribonucleotides that are or encode T cell strings. The present inventors have further determined that the T cell antigen fragments encoded by the present exemplary T cell antigen polyribonucleotides do not include sequence of 8 or more amino acids that is identical with the human proteome.

For the avoidance of doubt, the present disclosure includes exemplary polypeptide sequences and polyribonucleic acid sequences as described herein and/or as set forth in sequence identification numbers of the present disclosure, as well as sequences having at least 75%, at least 80%, at least 85%, at least 90%, at least 95%, at least 96%, at least 97%, at least 98%, or at least 99% identity thereto.

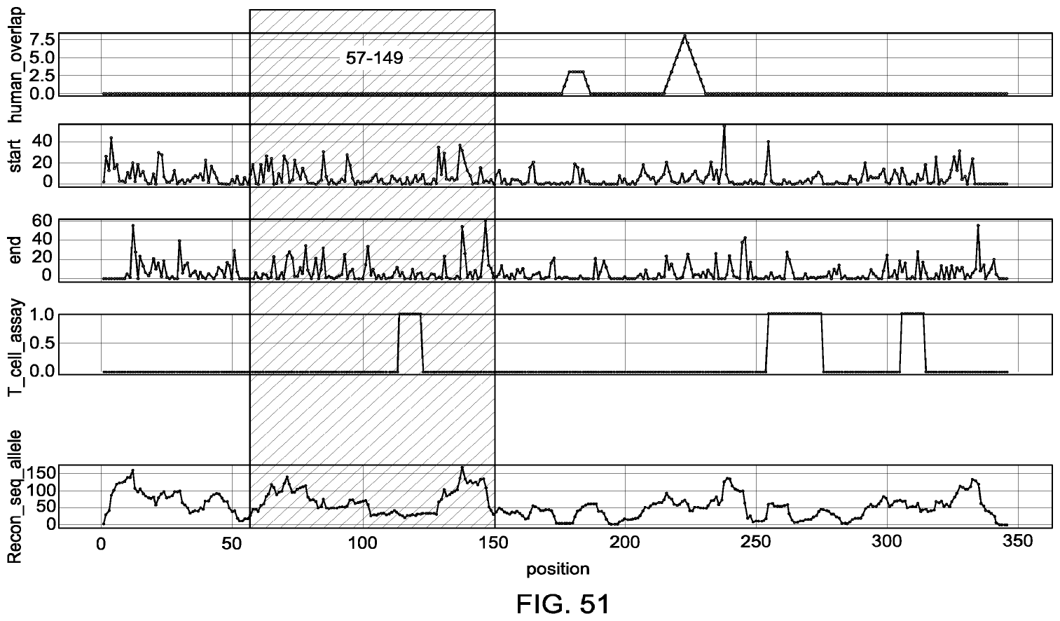

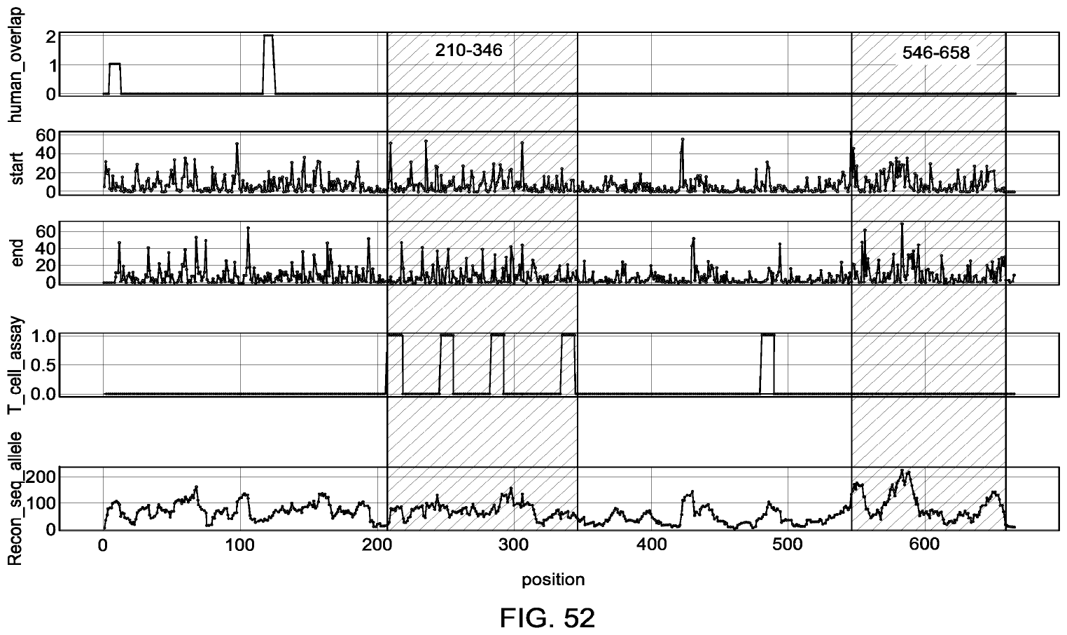

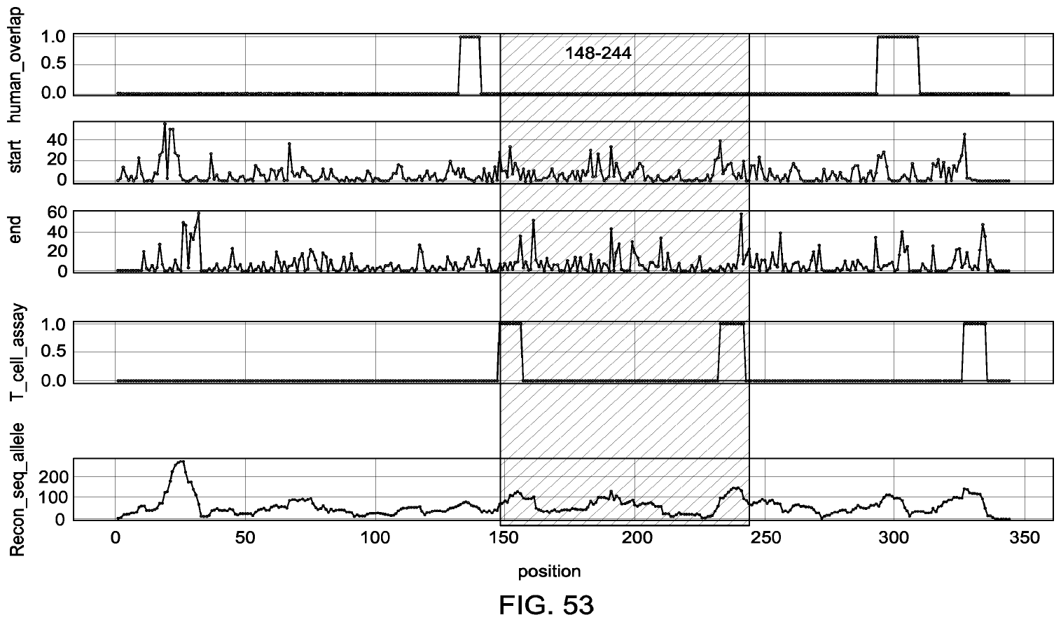

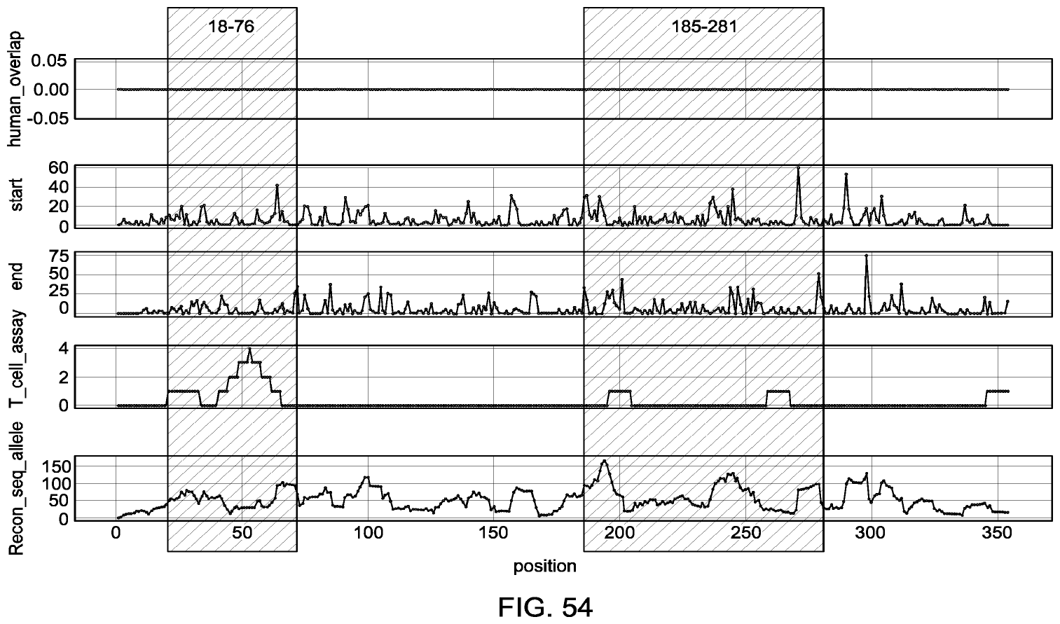

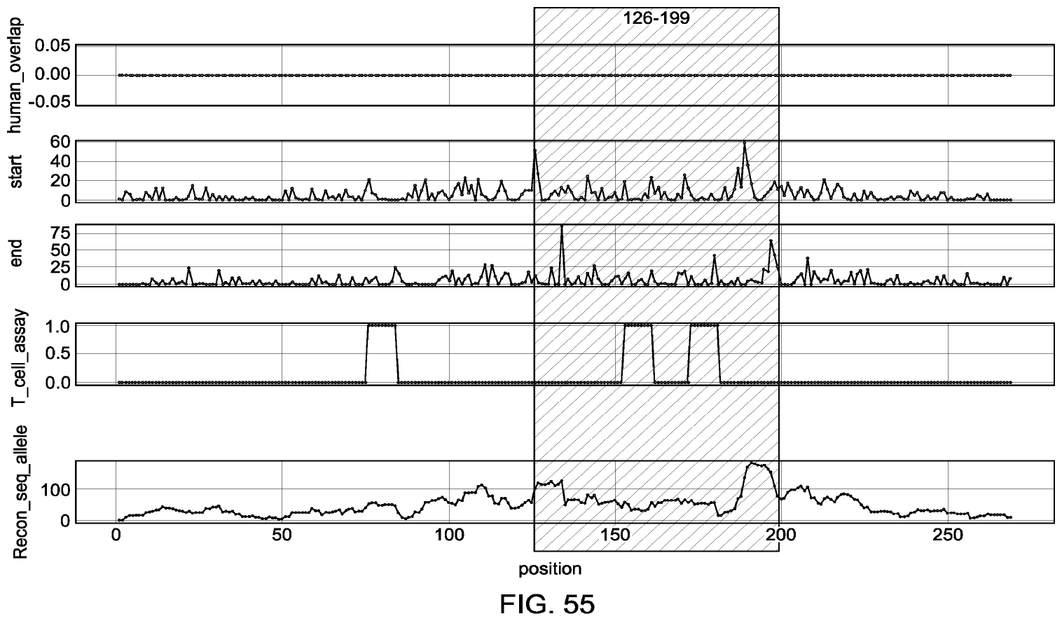

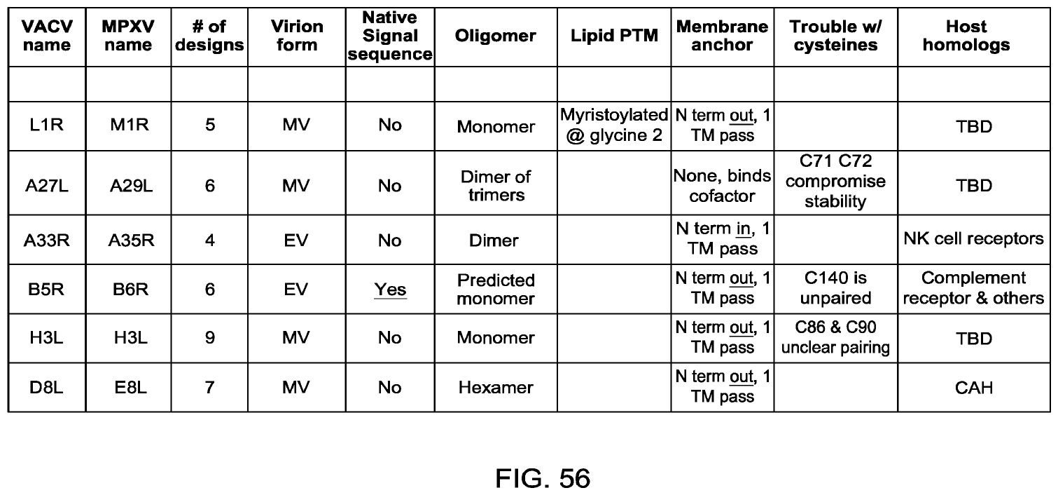

Exemplary fragments of T cell antigens that can be encoded by polyribonucleotides of the present disclosure include:
(i) an A45L fragment according to SEQ ID NO: 204 (amino acids 57-149 of an A45L reference sequence; see, e.g., FIG. 51), which can be encoded by an exemplary nucleic acid sequence according to SEQ ID NO: 205;
(ii) an Q1L fragment according to SEQ ID NO: 206 (amino acids 210-346 of an Q1L reference sequence; “Q1L-1”; see, e.g., FIG. 52), which can be encoded by an exemplary nucleic acid sequence according to SEQ ID NO: 207;
(iii) an Q1L fragment according to SEQ ID NO: 208 (amino acids 546-658 of an Q1L reference sequence; “Q1L-2”; see, e.g., FIG. 52), which can be encoded by an exemplary nucleic acid sequence according to SEQ ID NO: 209;
(iv) an B12R fragment according to SEQ ID NO: 210 (amino acids 148-244 of an B12R reference sequence; see, e.g., FIG. 53), which can be encoded by an exemplary nucleic acid sequence according to SEQ ID NO: 211;
(v) an C17L fragment according to SEQ ID NO: 212 (amino acids 18-76 of an C17L reference sequence; “C17L-1”; see, e.g., FIG. 54), which can be encoded by an exemplary nucleic acid sequence according to SEQ ID NO: 213;
(vi) an C17L fragment according to SEQ ID NO: 214 (amino acids 185-281 of an C17L reference sequence; “C17L-2”; see, e.g., FIG. 54), which can be encoded by an exemplary nucleic acid sequence according to SEQ ID NO: 215; and
(vii) an I3L fragment according to SEQ ID NO: 216 (amino acids 126-199 of an I3L reference sequence; see, e.g., FIG. 55), which can be encoded by an exemplary nucleic acid sequence according to SEQ ID NO: 217.

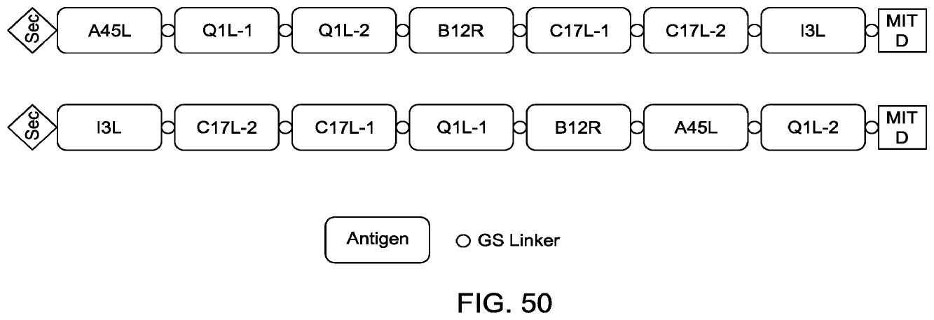

In one exemplary embodiment, a T cell string construct can include an HSV-1 signal polypeptide according to SEQ ID NO: 218, a string of T cell antigen fragments including fragments of A45L (SEQ ID NO: 204), Q1L (Q1L-1; SEQ ID NO: 206), Q1L (Q1L-2; SEQ ID NO: 208), B12R (SEQ ID NO: 210), C17L (C17L-1; SEQ ID NO: 212), C17L (C17L-2; SEQ ID NO: 214), and I3L (SEQ ID NO: 216), and MITD (SEQ ID NO: 219). T cell antigen fragments can be joined by linkers, e.g., linkers having a sequence according to SEQ ID NO: 176 or linkers having the sequence GGSGG (SEQ ID NO: 252). In some embodiments, a polyribonucleotide encoding a T cell string can encode a polypeptide according to SEQ ID NO: 220. (See, e.g., FIG. 50)

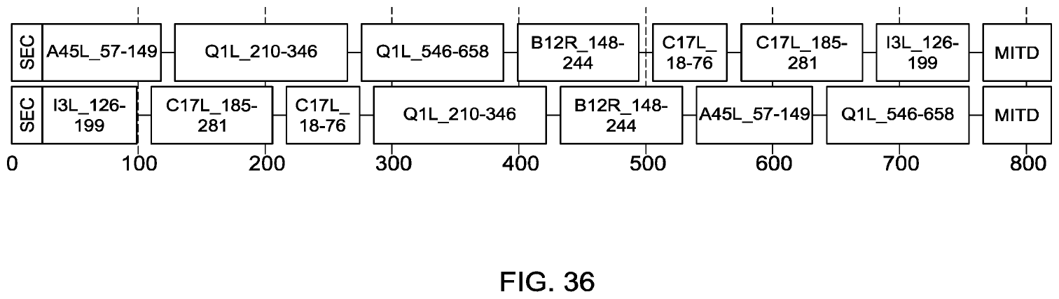

In one exemplary embodiment, a T cell string construct can include an HSV-1 signal polypeptide according to SEQ ID NO: 218, a string of T cell antigen fragments including fragments of I3L (SEQ ID NO: 216), C17L (C17L-2; SEQ ID NO: 214), C17L (C17L-1; SEQ ID NO: 212), Q1L (Q1L-1; SEQ ID NO: 206), B12R (SEQ ID NO: 210), A45L (SEQ ID NO: 204), and Q1L (Q1L-2; SEQ ID NO: 208), and MITD (SEQ ID NO: 219). T cell antigen fragments can be joined by linkers, e.g., linkers having a sequence according to SEQ ID NO: 176. In some embodiments, a polyribonucleotide encoding a T cell string can encode a polypeptide according to SEQ ID NO: 221. (See, e.g., FIG. 50 (schematic) and FIG. 36 (schematic with sequence lengths shown to scale))

### III. RNA Delivery Technologies

Provided polyribonucleotides may be delivered for therapeutic applications described herein using any appropriate methods known in the art, including, e.g., delivery as naked RNAs, or delivery mediated by viral and/or non-viral vectors, polymer-based vectors, lipid-based vectors, nanoparticles (e.g., lipid nanoparticles, polymeric nanoparticles, lipid-polymer hybrid nanoparticles, etc.), and/or peptide-based vectors. See, e.g., Wadhwa et al. “Opportunities and Challenges in the Delivery of mRNA-Based Vaccines” Pharmaceutics (2020) 102 (27 pages), the content of which is incorporated herein by reference, for information on various approaches that may be useful for delivery of polyribonucleotides described herein.

In some embodiments, one or more polyribonucleotides can be formulated with lipid nanoparticles for delivery (e.g., administration).

In some embodiments, lipid nanoparticles can be designed to protect polyribonucleotides from extracellular RNases and/or engineered for systemic delivery of the RNA to target cells (e.g., liver cells). In some embodiments, such lipid nanoparticles may be particularly useful to deliver polyribonucleotides when polyribonucleotides are intravenously or intramuscularly administered to a subject.

### A. Particles for Delivery of at Least One Polyribonucleotide

Polyribonucleotides provided herein can be delivered by particles. In the context of the present disclosure, the term “particle” relates to a structured entity formed by molecules or molecule complexes. In some embodiments, the term “particle” relates to a micro- or nano-sized structure, such as a micro- or nano-sized compact structure dispersed in a medium. In some embodiments, a particle is a nucleic acid containing particle such as a particle comprising a polyribonucleotide.

Electrostatic interactions between positively charged molecules such as polymers and lipids and a negatively charged nucleic acid (e.g., a polyribonucleotide) are involved in particle formation. This results in complexation and spontaneous formation of nucleic acid particles (e.g., ribonucleic acid particles). In some embodiments, a nucleic acid particle (e.g., ribonucleic acid particle) is a nanoparticle.

A “nucleic acid particle” (e.g., a ribonucleic acid particle) are particles that encompass or contain a nucleic acid, and are used to deliver a nucleic acid (e.g., a polyribonucleotide) to a target site of interest (e.g., cell, tissue, organ, and the like). A nucleic acid particle (e.g., a ribonucleic acid particle) may be formed from (i) at least one cationic or cationically ionizable lipid or lipid-like material, (ii) at least one cationic polymer such as protamine, or a mixture of (i) and (ii), and (iii) a nucleic acid (e.g., a polyribonucleotide). Nucleic acid particles (e.g., a ribonucleic acid particle) include lipid nanoparticles (LNPs) and lipoplexes (LPX).

In some embodiments, nucleic acid particles (e.g., ribonucleic acid particles) comprise more than one type of nucleic acid molecules (e.g., polyribonucleotides), where the molecular parameters of the nucleic acid molecules may be similar or different from each other, like with respect to molar mass or fundamental structural elements such as molecular architecture, capping, coding regions or other features.

In some embodiments, provided nucleic acid particles (e.g., ribonucleic acid particles) can comprise lipid nanoparticles. As used in the present disclosure, “nanoparticle” refers to a particle having an average diameter suitable for parenteral administration. In various embodiments, lipid nanoparticles can have an average size (e.g., mean diameter) of about 30 nm to about 150 nm, about 40 nm to about 150 nm, about 50 nm to about 150 nm, about 60 nm to about 130 nm, about 70 nm to about 110 nm, about 70 nm to about 100 nm, about 70 to about 90 nm, or about 70 nm to about 80 nm. In some embodiments, lipid nanoparticles in accordance with the present disclosure can have an average size (e.g., mean diameter) of about 50 nm to about 100 nm. In some embodiments, lipid nanoparticles may have an average size (e.g., mean diameter) of about 50 nm to about 150 nm. In some embodiments, lipid nanoparticles may have an average size (e.g., mean diameter) of about 60 nm to about 120 nm. In some embodiments, lipid nanoparticles in accordance with the present disclosure can have an average size (e.g., mean diameter) of about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

Nucleic acid particles (e.g., ribonucleic acid particles) described herein may exhibit a polydispersity index less than about 0.5, less than about 0.4, less than about 0.3, or about 0.2 or less. By way of example, the nucleic acid particles (e.g., ribonucleic acid particles) can exhibit a polydispersity index in a range of about 0.1 to about 0.3 or about 0.2 to about 0.3.

Nucleic acid particles (e.g., ribonucleic acid particles) described herein can be characterized by an “N/P ratio,” which is the molar ratio of cationic (nitrogen) groups (the “N” in N/P) in the cationic polymer to the anionic (phosphate) groups (the “P” in N/P) in RNA. It is understood that a cationic group is one that is either in cationic form (e.g., N*), or one that is ionizable to become cationic. Use of a single number in an N/P ratio (e.g., an N/P ratio of about 5) is intended to refer to that number over 1, e.g., an N/P ratio of about 5 is intended to mean 5:1. In some embodiments, a nucleic acid particle (e.g., a ribonucleic acid particle) described herein has an N/P ratio greater than or equal to 5. In some embodiments, a nucleic acid particle (e.g., a ribonucleic acid particle) described herein has an N/P ratio that is about 5, 6, 7, 8, 9, or 10. In some embodiments, an N/P ratio for a nucleic acid particle (e.g., a ribonucleic acid particle) described herein is from about 10 to about 50. In some embodiments, an N/P ratio for a nucleic acid particle (e.g., a ribonucleic acid particle) described herein is from about 10 to about 70. In some embodiments, an N/P ratio for a nucleic acid particle (e.g., a ribonucleic acid particle) described herein is from about 10 to about 120.

Nucleic acid particles (e.g., ribonucleic acid particles) described herein can be prepared using a wide range of methods that may involve obtaining a colloid from at least one cationic or cationically ionizable lipid or lipid-like material and/or at least one cationic polymer and mixing the colloid with nucleic acid to obtain nucleic acid particles.

The term “colloid” as used herein relates to a type of homogeneous mixture in which dispersed particles do not settle out. The insoluble particles in the mixture can be microscopic, with particle sizes between 1 and 1000 nanometers. The mixture may be termed a colloid or a colloidal suspension. Sometimes the term “colloid” only refers to the particles in the mixture and not the entire suspension.

The term “average diameter” or “mean diameter” refers to the mean hydrodynamic diameter of particles as measured by dynamic laser light scattering (DLS) with data analysis using the so-called cumulant algorithm, which provides as results the so-called Z-average with the dimension of a length, and the polydispersity index (PI), which is dimensionless (Koppel, D., J. Chem. Phys. 57, 1972, pp 4814-4820, ISO 13321, which is herein incorporated by reference). Here “average diameter,” “mean diameter,” “diameter,” or “size” for particles is used synonymously with this value of the Z-average.

The “polydispersity index” is preferably calculated based on dynamic light scattering measurements by the so-called cumulant analysis as mentioned in the definition of the “average diameter.” Under certain prerequisites, it can be taken as a measure of the size distribution of an ensemble of ribonucleic acid nanoparticles (e.g., ribonucleic acid nanoparticles).

Different types of nucleic acid particles have been described previously to be suitable for delivery of nucleic acid in particulate form (e.g. Kaczmarek, J. C. et al., 2017, Genome Medicine 9, 60, which is herein incorporated by reference). For non-viral nucleic acid delivery vehicles, nanoparticle encapsulation of nucleic acid physically protects nucleic acid from degradation and, depending on the specific chemistry, can aid in cellular uptake and endosomal escape.

The present disclosure describes particles comprising a nucleic acid (e.g., a polyribonucleotide), at least one cationic or cationically ionizable lipid or lipid-like material, and/or at least one cationic polymer which associate with the nucleic acid (e.g., a polyribonucleotide) to form nucleic acid particles (e.g., ribonucleic acid particles, e.g., ribonucleic acid nanoparticles) and compositions comprising such particles. The nucleic acid particles (e.g., ribonucleic acid particles, e.g., ribonucleic acid nanoparticles) may comprise a nucleic acid (e.g., a polyribonucleotide) which is complexed in different forms by non-covalent interactions to the particle. The particles described herein are not viral particles, in particular, they are not infectious viral particles, i.e., they are not able to virally infect cells.

Some embodiments described herein relate to compositions, methods and uses involving more than one, e.g., 2, 3, 4, 5, 6 or even more nucleic acid species (e.g., polyribonucleotide species).

In a nucleic acid particle (e.g., ribonucleic acid particle, e.g., ribonucleic acid nanoparticle) formulation, it is possible that each nucleic acid species (e.g., polyribonucleotide species) is separately formulated as an individual nucleic acid particle (e.g., ribonucleic acid particle, e.g., ribonucleic acid nanoparticle) formulation. In that case, each individual nucleic acid particle (e.g., ribonucleic acid particle, e.g., ribonucleic acid nanoparticle) formulation will comprise one nucleic acid species (e.g., polyribonucleotide species). The individual nucleic acid particle (e.g., ribonucleic acid particle, e.g., ribonucleic acid nanoparticle) formulations may be present as separate entities, e.g., in separate containers. Such formulations are obtainable by providing each nucleic acid species (e.g., polyribonucleotide species) separately (typically each in the form of a nucleic acid-containing solution) together with a particle-forming agent, thereby allowing the formation of particles. Respective particles will contain exclusively the specific nucleic acid species (e.g., polyribonucleotide species) that is being provided when the particles are formed (individual particulate formulations).

In some embodiments, a composition such as a pharmaceutical composition comprises more than one individual nucleic acid particle (e.g., ribonucleic acid particle, e.g., ribonucleic acid nanoparticle) formulation. Respective pharmaceutical compositions are referred to as “mixed particulate formulations.” Mixed particulate formulations according to the invention are obtainable by forming, separately, individual nucleic acid particle (e.g., ribonucleic acid particle, e.g., ribonucleic acid nanoparticle) formulations, as described above, followed by a step of mixing of the individual nucleic acid particle (e.g., ribonucleic acid particle, e.g., ribonucleic acid nanoparticle) formulations. By the step of mixing, a formulation comprising a mixed population of nucleic acid-containing particles is obtainable. Individual nucleic acid particle (e.g., ribonucleic acid particle, e.g., ribonucleic acid nanoparticle) populations may be together in one container, comprising a mixed population of individual nucleic acid particle (e.g., ribonucleic acid particle, e.g., ribonucleic acid nanoparticle) formulations.

Alternatively, it is possible that different nucleic acid species (e.g., polyribonucleotide species) are formulated together as a “combined particulate formulation.” Such formulations are obtainable by providing a combined formulation (typically combined solution) of different nucleic acid species (e.g., polyribonucleotide species) species together with a particle-forming agent, thereby allowing the formation of particles. As opposed to a “mixed particulate formulation,” a “combined particulate formulation” will typically comprise particles that comprise more than one nucleic acid species (e.g., polyribonucleotide species) species. In a combined particulate composition different nucleic acid species (e.g., polyribonucleotide species) are typically present together in a single particle.

In some embodiments, nucleic acids (e.g., polyribonucleotides), when present in provided nucleic acid particles (e.g., ribonucleic acid particles, e.g., lipid nanoparticles) are resistant in aqueous solution to degradation with a nuclease.

In some embodiments, nucleic acid particles (e.g., ribonucleic acid particles) are lipid nanoparticles. In some embodiments, lipid nanoparticles are liver-targeting lipid nanoparticles. In some embodiments, lipid nanoparticles are cationic lipid nanoparticles comprising one or more cationic lipids (e.g., ones described herein). In some embodiments, cationic lipid nanoparticles may comprise at least one cationic lipid, at least one polymer-conjugated lipid, and at least one helper lipid (e.g., at least one neutral lipid).

### 1. Cationic Polymeric Materials

Cationic polymers have been recognized as useful for developing such delivery vehicles, as reported in PCT App. Pub. No. WO 2021/001417, the entirety of which is incorporated herein by reference. As used herein, the term “polymer” refers to a composition comprising one or more molecules that comprise repeating units of one or more monomers. As used herein, “polymer,” “polymeric material,” and “polymer composition” are used interchangeably, and unless otherwise specified, refer to a composition of polymer molecules. A person of skill in the art will appreciate that a polymer composition comprises polymer molecules having molecules of different lengths (e.g., comprising varying amounts of monomers). Polymer compositions described herein are characterized by one or more of a normalized molecular weight (Mn), a weight average molecular weight (Mw), and/or a polydispersity index (PDI). In some embodiments, such repeat units can all be identical (a “homopolymer”); alternatively, in some cases, there can be more than one type of repeat unit present within the polymeric material (a “heteropolymer” or a “copolymer”). In some cases, a polymer is biologically derived, e.g., a biopolymer such as a protein. In some cases, additional moieties can also be present in the polymeric material, for example targeting moieties such as those described herein.

In some embodiments, a polymer utilized in accordance with the present disclosure may be a copolymer. Repeat units forming the copolymer can be arranged in any fashion. For example, in some embodiments, repeat units can be arranged in a random order; alternatively or additionally, in some embodiments, repeat units may be arranged in an alternating order, or as a “block” copolymer, e.g., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In some embodiments, a polymeric material for use in accordance with the present disclosure is biocompatible. In some embodiments, a biocompatible material is biodegradable, e.g., is able to degrade, chemically and/or biologically, within a physiological environment, such as within the body.

In some embodiments, a polymeric material may be or comprise protamine or polyalkyleneimine.

As those skilled in the art are aware term “protamine” is often used to refer to any of various strongly basic proteins of relatively low molecular weight that are rich in arginine and are found associated especially with DNA in place of somatic histones in the sperm cells of various animals (e.g., fish). In particular, the term “protamine” is often used to refer to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

In some embodiments, the term “protamine” as used herein is refers to a protamine amino acid sequence obtained or derived from natural or biological sources, including fragments thereof and/or multimeric forms of said amino acid sequence or fragment thereof, as well as (synthesized) polypeptides which are artificial and specifically designed for specific purposes and cannot be isolated from native or biological sources.

In some embodiments, a polyalkyleneimine comprises polyethylenimine (PEI) and/or polypropylenimine. In some embodiments, a preferred polyalkyleneimine is polyethyleneimine (PEI). In some embodiments, the average molecular weight of PEI is preferably 0.75×10² to 10⁷ Da, preferably 1000 to 10⁵ Da, more preferably 10000 to 40000 Da, more preferably 15000 to 30000 Da, even more preferably 20000 to 25000 Da.

Cationic materials (e.g., polymeric materials, including polycationic polymers) contemplated for use herein include those which are able to electrostatically bind nucleic acid. In some embodiments, cationic polymeric materials contemplated for use herein include any cationic polymeric materials with which nucleic acid can be associated, e.g., by forming complexes with the nucleic acid or forming vesicles in which the nucleic acid is enclosed or encapsulated.

In some embodiments, particles described herein may comprise polymers other than cationic polymers, e.g., non-cationic polymeric materials and/or anionic polymeric materials. Collectively, anionic and neutral polymeric materials are referred to herein as non-cationic polymeric materials.

### 2. Lipid Particles

The terms “lipid” and “lipid-like material” are used herein to refer to molecules that comprise one or more hydrophobic moieties or groups and optionally also one or more hydrophilic moieties or groups. Molecules comprising hydrophobic moieties and hydrophilic moieties are also frequently denoted as amphiphiles. Lipids are usually poorly soluble in water. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). In some embodiments, hydrophilic groups may comprise polar and/or charged groups and include carbohydrates, phosphate, carboxylic, sulfate, amino, sulfhydryl, nitro, hydroxyl, and other like groups.

Lipid nanoparticles (also referred to as “lipid nanoparticles”) of the present disclosure comprise (i) a cationic lipid; (ii) a polymer-conjugated lipid, and (iii) one or more helper lipids. Lipid nanoparticles described herein are useful for the delivery of nucleic acid cargo (e.g., a polyribonucleotide) into the cell of a subject. In some embodiments, lipid nanoparticles comprising a nucleic acid (e.g., a polyribonucleotide) described herein are useful for causing increased expression of a protein (e.g., an antigen or fragment thereof) in a subject. In some embodiments, lipid nanoparticles comprising a nucleic acid (e.g., a polyribonucleotide) described herein are useful for causing a pharmacological effect induced by expression of a protein in a subject. Lipid nanoparticles described herein are characterized by molar percentage (mol %) of components in the lipid nanoparticle. A mol % used in reference to a lipid component of a lipid nanoparticle is relative to the total other lipid components in the lipid nanoparticle.

a. Cationic lipids

As described herein, lipid nanoparticles of the present disclosure comprise a cationic lipid. In some embodiments, a lipid nanoparticle for delivery of at least one polyribonucleotide described herein comprises a cationic lipid. A cationic lipid, as described herein, is a lipid that is positively charged or is ionizable, such that the cationic lipid will become positively charged when subjected to particular physiological conditions, e.g., a pH of about 7.4 or less, and can promote lipid aggregation. In some embodiments, a cationic lipid is a lipid comprising one or more amine groups which bear or are capable of bearing a positive charge.

In some embodiments, a cationic lipid may comprise a cationic, meaning positively charged, headgroup. In some embodiments, a cationic lipid may have a hydrophobic domain (e.g., one or more domains of a neutral lipid or an anionic lipid) provided that the cationic lipid has a net positive charge. In some embodiments, a cationic lipid comprises a polar headgroup, which in some embodiments may comprise one or more amine derivatives such as primary, secondary, and/or tertiary amines, quaternary ammonium, various combinations of amines, amidinium salts, or guanidine and/or imidazole groups as well as pyridinium, piperizine and amino acid headgroups such as lysine, arginine, ornithine and/or tryptophan. In some embodiments, a polar headgroup of a cationic lipid comprises one or more amine derivatives. In some embodiments, a polar headgroup of a cationic lipid comprises a quaternary ammonium. In some embodiments, a headgroup of a cationic lipid may comprise multiple cationic charges. In some embodiments, a headgroup of a cationic lipid comprises one cationic charge.

In some embodiments, a cationic lipid is selected from 1,2-dimyristoyl-sn-glycero-3-ethylphosphocholine (DMEPC); 2-dimyristoyl-3-trimethylammonium propane (DMTAP); dioleyl ether phosphatidylcholine (DOEPC); N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3dioleoyloxy)propyl)-N,N,N-trimethylammonium chloride (DOTAP); 3-(N—(N′,N′dimethylaminoethane)-carbamoyl)cholesterol (DC-Chol), N-(1-(2,3-dioleoyloxy)propyl)N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE).

In some embodiments, a cationic lipid is one provided in WO2012/016184, which is incorporated herein by reference in its entirety. For example, in some embodiments, a cationic lipid is selected from 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

In some embodiments, a cationic lipid is one provided in WO2020/219941, WO2017/075531, WO2016/176330, WO2017/049245, or U.S. Pat. No. 9,670,152, each of which is incorporated herein by reference in its entirety.

In some embodiments, a cationic lipid is a compound of Formula I:

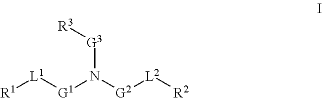

or a pharmaceutically acceptable salt thereof, wherein:
one of L¹ or L² is —OC(O)—, —C(O)O—, —C(O)—, —O—, —S(O)ₓ—, —S—S—, —C(O)S—, SC(O)—, —NR^{a}C(O)—, —C(O)NR^{a}—, —NR^{a}C(O)NR^{a}—, —OC(O)NR^{a}— or —NR^{a}C(O)O—, and the other of L¹ or L² is —OC(O)—, —C(O)O—, —C(O)—, —O—, —S(O)X—, —S—S—, —C(O)S—, SC(O)—, —NR^{a}C(O)—, —C(O)NR^{a}—, —NR^{a}C(O)NR^{a}—, —OC(O)NR^{a}—, —NR^{a}C(O)O—, or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(O)OR⁴, —OC(O)R⁴ or —R⁵C(O)R⁴;
R⁴ is C₁-C₁₂ alkyl;
R⁵ is H or C₁-C₆ alkyl; and
x is 0, 1 or 2.

In some embodiments, one of L¹ or L² is —OC(O)— or —C(O)O—. In some embodiments, each of L¹ and L² is —OC(O)— or —C(O)O—.

In some embodiments, G¹ is C₁-C₁₂ alkylene. In some embodiments, G² is C₁-C₁₂alkylene. In some embodiments G¹ and G² are each independently C₁-C₁₂ alkylene. In some embodiments G¹ and G² are each independently C₅-C₁₂ alkylene.

In some embodiments, G³ is C₁-C₂₄ alkylene. In some embodiments, G³ is C₁-C₆ alkylene.

In some embodiments, R¹ and R² are each independently selected from:

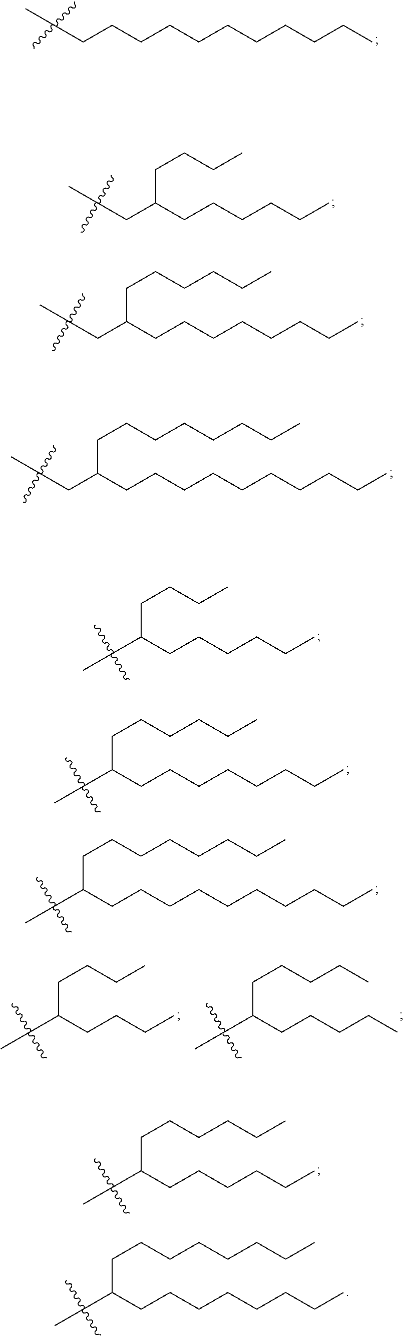

In some embodiments, R³ is OH.

In some embodiments, each of L¹ and L² is —OC(O)—, G¹ and G² are each independently C₅-C₁₂ alkylene, G³ is C₁-C₆ alkylene, R³ is OH, and R¹ and R² are each independently selected from:

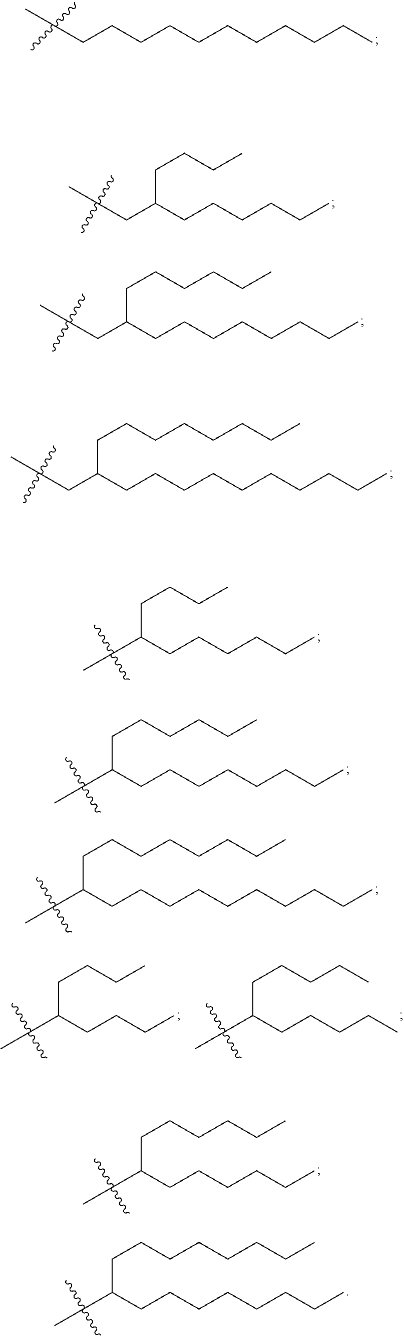

In some embodiments, a cationic lipid is a compound of Formula Ia or Ib

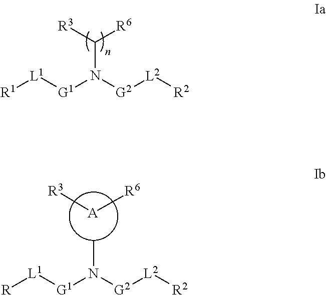

or a pharmaceutically acceptable salt thereof, where n is an integer from 1 to 15, A is C₃-C₈ cycloaliphatic, each R6 is independently selected from H, OH, and C₁-C₂₄ aliphatic, and wherein R1, R2, R3, L1, L2, G1, and G2 are as described in classes and subclasses herein, both singly and in combination.

In some embodiments, a positively charged lipid structure described herein may also include one or more other components that may be typically used in the formation of vesicles (e.g. for stabilization). Examples of such other components includes, without being limited thereto, fatty alcohols, fatty acids, and/or cholesterol esters or any other pharmaceutically acceptable excipients which may affect the surface charge, the membrane fluidity and assist in the incorporation of the lipid into the lipid assembly. Examples of sterols include cholesterol, cholesteryl hemisuccinate, cholesteryl sulfate, or any other derivatives of cholesterol. Preferably, the at least one cationic lipid comprises DMEPC and/or DOTMA.

In some embodiments, a cationic lipid is ionizable such that it can exist in a positively charged form or neutral form depending on pH. Such ionization of a cationic lipid can affect the surface charge of the lipid particle under different pH conditions, which in some embodiments may influence plasma protein absorption, blood clearance, and/or tissue distribution as well as the ability to form endosomolytic non-bilayer structures. Accordingly, in some embodiments, a cationic lipid may be or comprise a pH responsive lipid. In some embodiments a pH responsive lipid is a fatty acid derivative or other amphiphilic compound which is capable of forming a lyotropic lipid phase, and which has a pKa value between pH 5 and pH 7.5. This means that the lipid is uncharged at a pH above the pKa value and positively charged below the pKa value. In some embodiments, a pH responsive lipid may be used in addition to or instead of a cationic lipid for example by binding one or more polyribonucleotides to a lipid or lipid mixture at low pH. pH responsive lipids include, but are not limited to, 1,2-dioieyioxy-3-dimethylamino-propane (DODMA).

In some embodiments, a lipid nanoparticle may comprise one or more cationic lipids as described in WO 2017/075531 (e.g., as presented in Tables 1 and 3 therein) and WO 2018/081480 (e.g., as presented in Tables 1-4 therein), the entire contents of each of which are incorporated herein by reference for the purposes described herein.

In some embodiments, a cationic lipid that may be useful in accordance with the present disclosure is an amino lipid comprising a titratable tertiary amino head group linked via ester bonds to at least two saturated alkyl chains, which ester bonds can be hydrolyzed easily to facilitate fast degradation and/or excretion via renal pathways. In some embodiments, such an amino lipid has an apparent pKa of about 6.0-6.5 (e.g., in one embodiment with an apparent pKa of approximately 6.25), resulting in an essentially fully positively charged molecule at an acidic pH (e.g., pH 5). In some embodiments, such an amino lipid, when incorporated in lipid nanoparticle, can confer distinct physicochemical properties that regulate particle formation, cellular uptake, fusogenicity and/or endosomal release of polyribonucleotide(s). In some embodiments, introduction of an aqueous RNA solution to a lipid mixture comprising such an amino lipid at pH 4.0 can lead to an electrostatic interaction between the negatively charged RNA backbone and the positively charged cationic lipid. Without wishing to be bound by any particular theory, such electrostatic interaction leads to particle formation coincident with efficient encapsulation of RNA drug substance. After RNA encapsulation, adjustment of the pH of the medium surrounding the resulting lipid nanoparticle to a more neutral pH (e.g., pH 7.4) results in neutralization of the surface charge of the lipid nanoparticle. When all other variables are held constant, such charge-neutral particles display longer in vivo circulation lifetimes and better delivery to hepatocytes compared to charged particles, which are rapidly cleared by the reticuloendothelial system. Upon endosomal uptake, the low pH of the endosome renders lipid nanoparticle comprising such an amino lipid fusogenic and allows the release of the RNA into the cytosol of the target cell.

In some embodiments, a cationic lipid that may be useful in accordance with the present disclosure has one of the structures set forth in Table 5 below:

TABLE 5
Exemplary cationic lipids

| No. | Structure |
|---|---|
| I-1 | 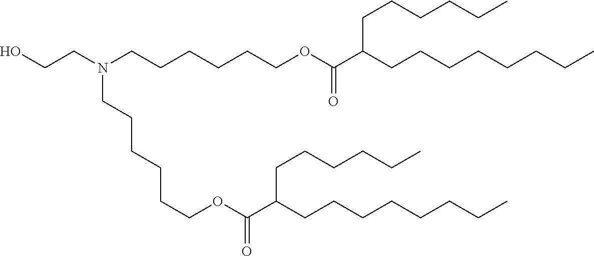 [SMILES: CCCCCCCCC(CCCCCC)C(=O)OCCCCCCN(CCO)CCCCCCOC(=O)C(CCCCCC)CCCCCCCC] |
| I-2 | 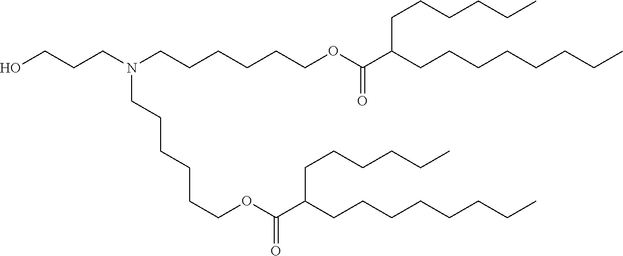 [SMILES: CCCCCCCCC(CCCCCC)C(=O)OCCCCCCN(CCCO)CCCCCCOC(=O)C(CCCCCC)CCCCCCCC] |
| I-3 | 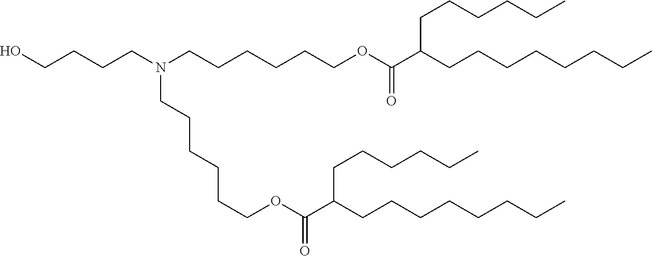 [SMILES: CCCCCCCCC(CCCCCC)C(=O)OCCCCCCN(CCCCO)CCCCCCOC(=O)C(CCCCCC)CCCCCCCC] |
| I-4 | 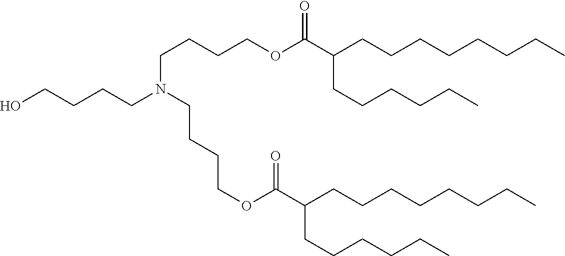 [SMILES: CCCCCCCCC(CCCCCC)C(=O)OCCCCN(CCCCO)CCCCOC(=O)C(CCCCCC)CCCCCCCC] |
| I-5 | 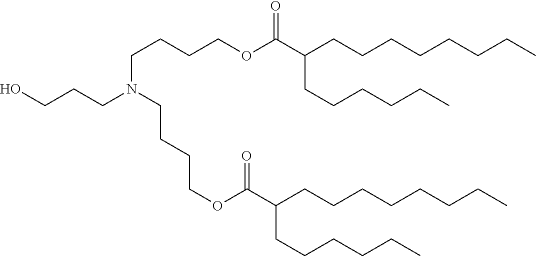 [SMILES: CCCCCCCCC(CCCCCC)C(=O)OCCCCN(CCCO)CCCCOC(=O)C(CCCCCC)CCCCCCCC] |
| I-6 | 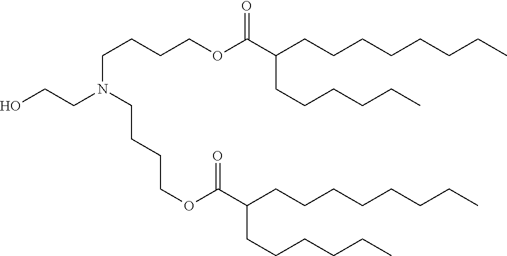 [SMILES: CCCCCCCCC(CCCCCC)C(=O)OCCCCN(CCO)CCCCOC(=O)C(CCCCCC)CCCCCCCC] |
| I-7 | 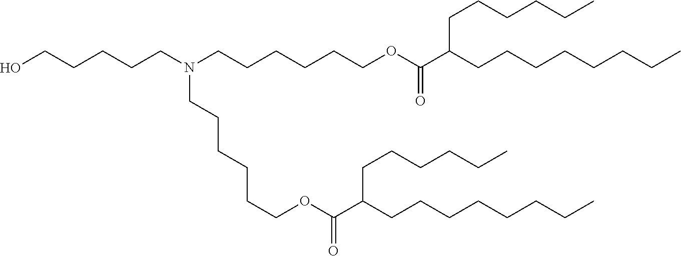 [SMILES: CCCCCCCCC(CCCCCC)C(=O)OCCCCCCN(CCCCCO)CCCCCCOC(=O)C(CCCCCC)CCCCCCCC] |
| I-8 | 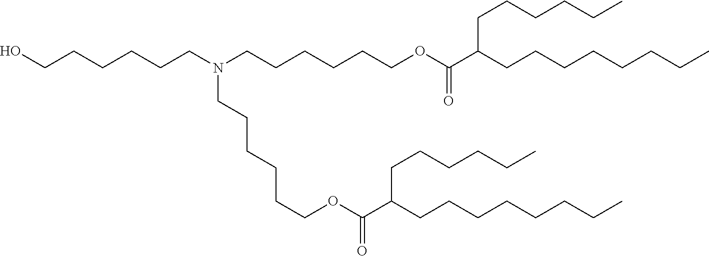 [SMILES: CCCCCCCCC(CCCCCC)C(=O)OCCCCCCN(CCCCCCO)CCCCCCOC(=O)C(CCCCCC)CCCCCCCC] |
| I-9 | 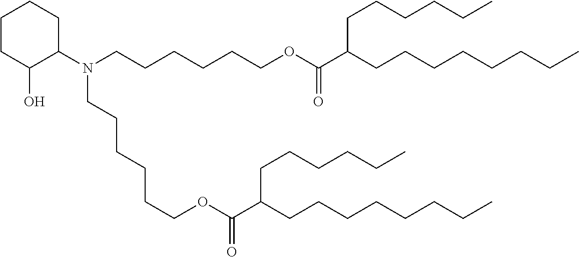 [SMILES: CCCCCCCCC(CCCCCC)C(=O)OCCCCCCN(CCCCCCOC(=O)C(CCCCCC)CCCCCCCC)C1CCCCC1O] |
| I-10 | 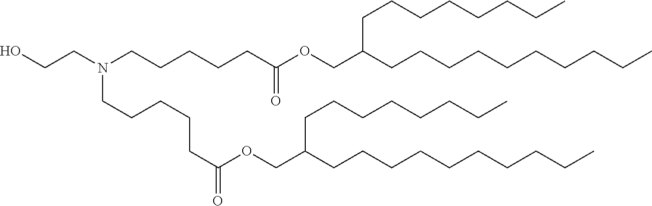 [SMILES: CCCCCCCCCCC(CCCCCCCC)COC(=O)CCCCCN(CCO)CCCCCC(=O)OCC(CCCCCCCC)CCCCCCCCCC] |
| I-11 | 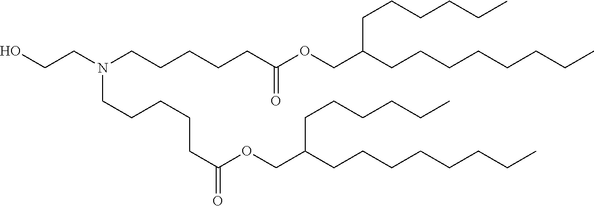 [SMILES: CCCCCCCCC(CCCCCC)COC(=O)CCCCCN(CCO)CCCCCC(=O)OCC(CCCCCC)CCCCCCCC] |
| I-12 | 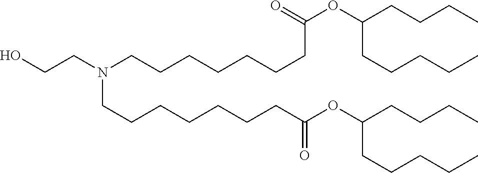 [SMILES: CCCCCC(CCCC)OC(=O)CCCCCCCN(CCO)CCCCCCCC(=O)OC(CCCC)CCCCC] |
| I-13 | 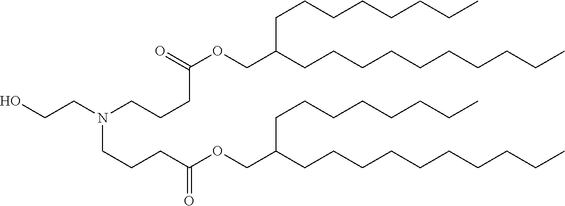 [SMILES: CCCCCCCCCCC(CCCCCCCC)COC(=O)CCCN(CCO)CCCC(=O)OCC(CCCCCCCC)CCCCCCCCCC] |
| I-14 | 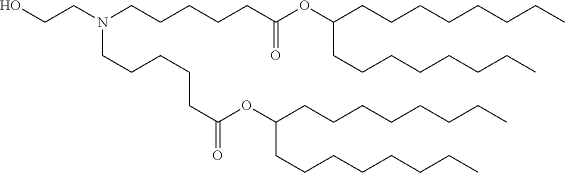 [SMILES: CCCCCCCCC(CCCCCCCC)OC(=O)CCCCCN(CCO)CCCCCC(=O)OC(CCCCCCCC)CCCCCCCC] |
| I-15 | 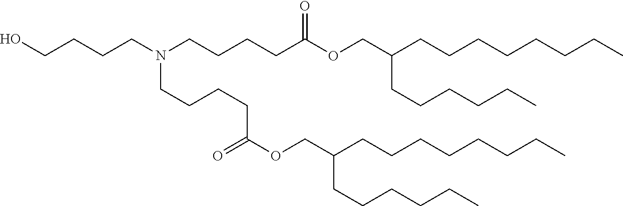 [SMILES: CCCCCCCCC(CCCCCC)COC(=O)CCCCN(CCCCO)CCCCC(=O)OCC(CCCCCC)CCCCCCCC] |
| I-16 | 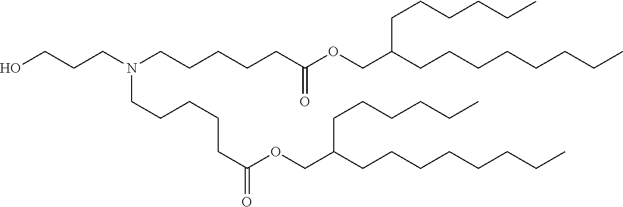 [SMILES: CCCCCCCCC(CCCCCC)COC(=O)CCCCCN(CCCO)CCCCCC(=O)OCC(CCCCCC)CCCCCCCC] |
| I-17 | 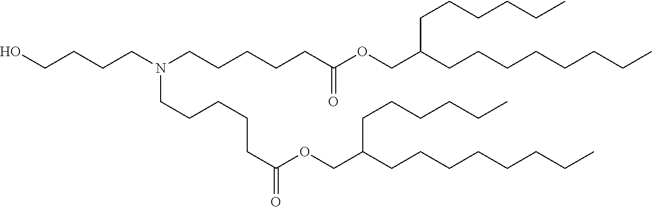 [SMILES: CCCCCCCCC(CCCCCC)COC(=O)CCCCCN(CCCCO)CCCCCC(=O)OCC(CCCCCC)CCCCCCCC] |
| I-18 | 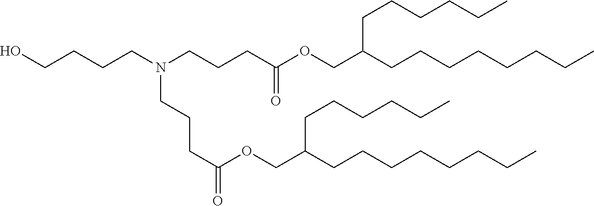 [SMILES: CCCCCCCCC(CCCCCC)COC(=O)CCCN(CCCCO)CCCC(=O)OCC(CCCCCC)CCCCCCCC] |
| I-19 | 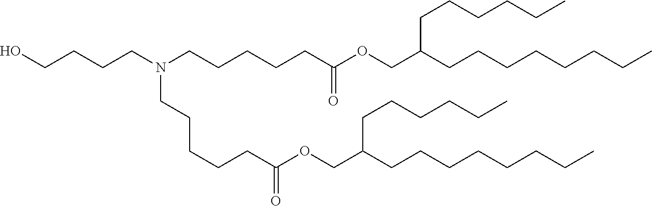 [SMILES: CCCCCCCCC(CCCCCC)COC(=O)CCCCCN(CCCCO)CCCCCC(=O)OCC(CCCCCC)CCCCCCCC] |
| I-20 | 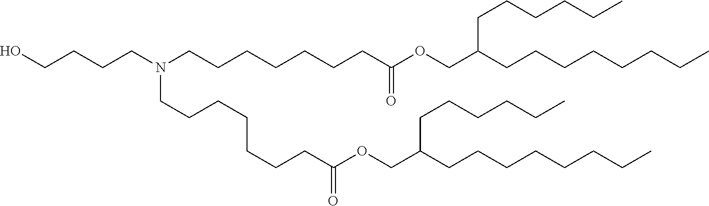 [SMILES: CCCCCCCCC(CCCCCC)COC(=O)CCCCCCCN(CCCCO)CCCCCCCC(=O)OCC(CCCCCC)CCCCCCCC] |
| I-21 | 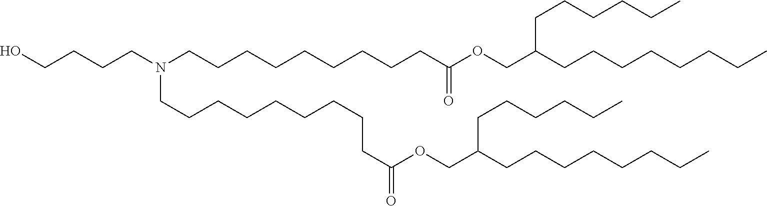 [SMILES: CCCCCCCCC(CCCCCC)COC(=O)CCCCCCCCCN(CCCCO)CCCCCCCCCC(=O)OCC(CCCCCC)CCCCCCCC] |
| I-22 | 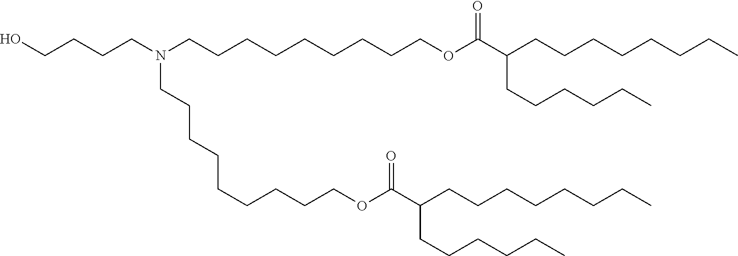 [SMILES: CCCCCCCCC(CCCCCC)C(=O)OCCCCCCCCCN(CCCCO)CCCCCCCCCOC(=O)C(CCCCCC)CCCCCCCC] |
| I-23 | 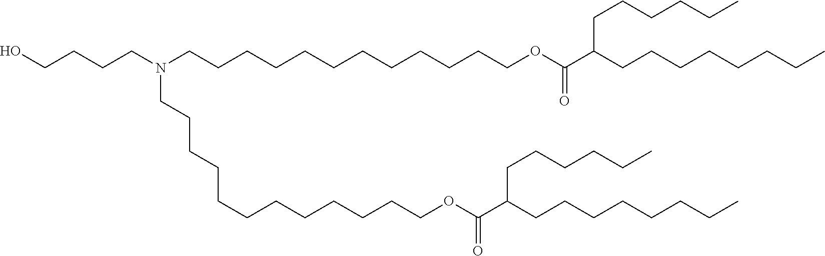 [SMILES: CCCCCCCCC(CCCCCC)C(=O)OCCCCCCCCCCCCN(CCCCO)CCCCCCCCCCCCOC(=O)C(CCCCCC)CCCCCCCC] |
| I-24 | 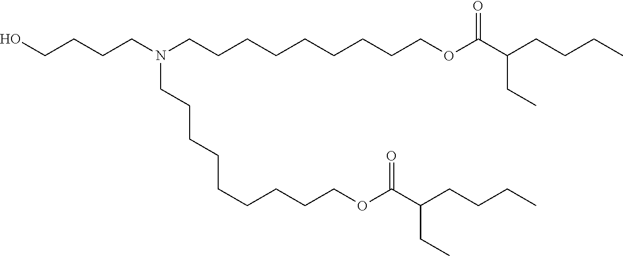 [SMILES: CCCCC(CC)C(=O)OCCCCCCCCCN(CCCCO)CCCCCCCCCOC(=O)C(CC)CCCC] |
| I-25 | 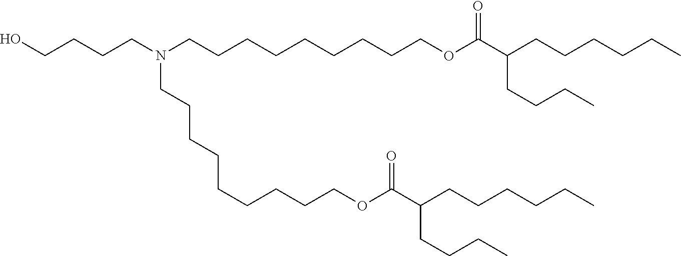 [SMILES: CCCCCCC(CCCC)C(=O)OCCCCCCCCCN(CCCCO)CCCCCCCCCOC(=O)C(CCCC)CCCCCC] |
| I-26 | 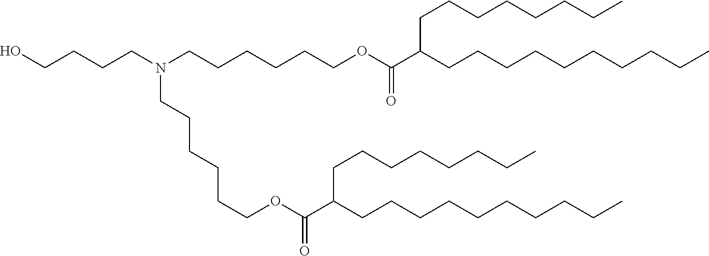 [SMILES: CCCCCCCCCCC(CCCCCCCC)C(=O)OCCCCCCN(CCCCO)CCCCCCOC(=O)C(CCCCCCCC)CCCCCCCCCC] |
| I-27 | 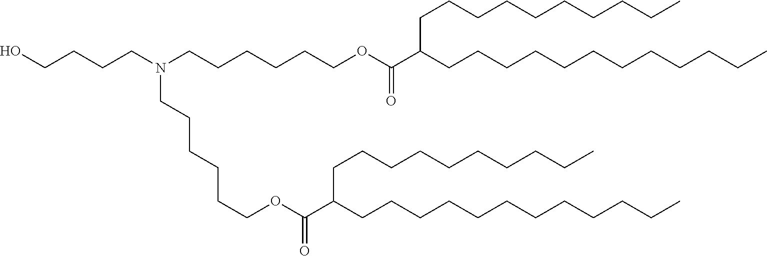 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| I-28 | 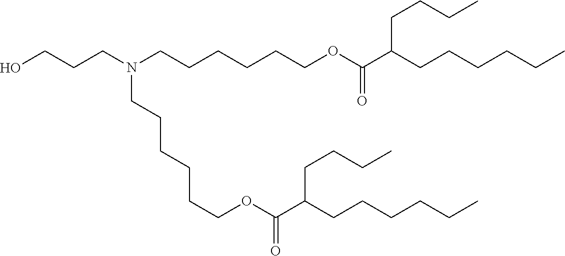 [SMILES: CCCCCCC(CCCC)C(=O)OCCCCCCN(CCCO)CCCCCCOC(=O)C(CCCC)CCCCCC] |
| I-29 | 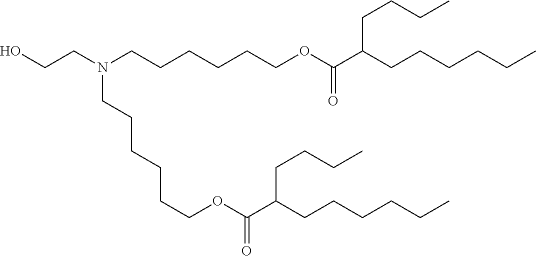 [SMILES: CCCCCCC(CCCC)C(=O)OCCCCCCN(CCO)CCCCCCOC(=O)C(CCCC)CCCCCC] |
| I-30 | 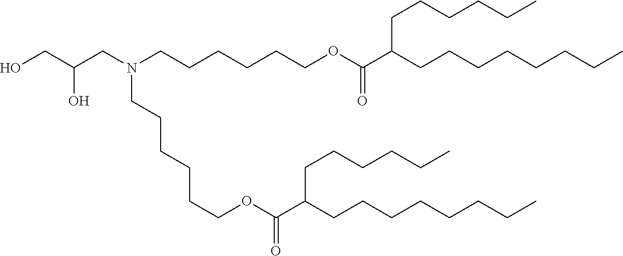 [SMILES: CCCCCCCCC(CCCCCC)C(=O)OCCCCCCN(CCCCCCOC(=O)C(CCCCCC)CCCCCCCC)CC(O)CO] |
| I-31 | 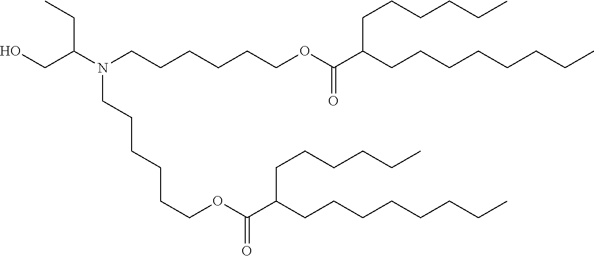 [SMILES: CCCCCCCCC(CCCCCC)C(=O)OCCCCCCN(CCCCCCOC(=O)C(CCCCCC)CCCCCCCC)C(CC)CO] |
| I-32 | 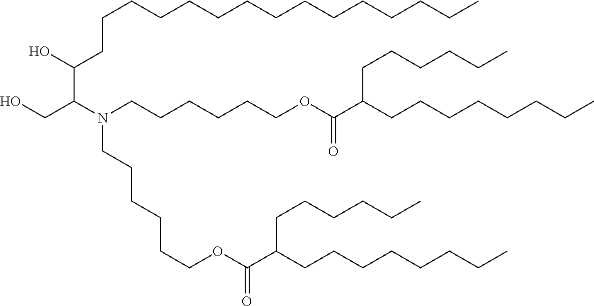 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| I-33 | 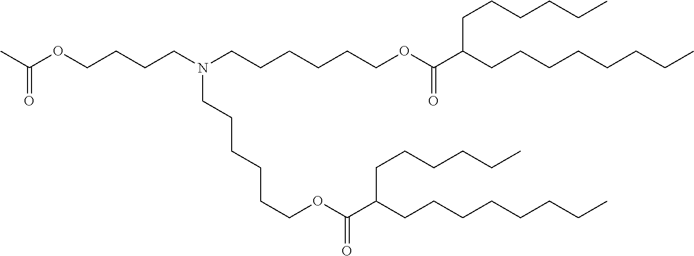 [SMILES: CCCCCCCCC(CCCCCC)C(=O)OCCCCCCN(CCCCCCOC(=O)C(CCCCCC)CCCCCCCC)CCCCOC(C)=O] |
| I-34 | 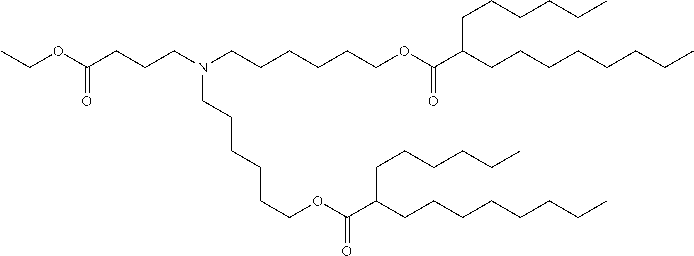 [SMILES: CCCCCCCCC(CCCCCC)C(=O)OCCCCCCN(CCCCCCOC(=O)C(CCCCCC)CCCCCCCC)CCCC(=O)OCC] |
| I-35 | 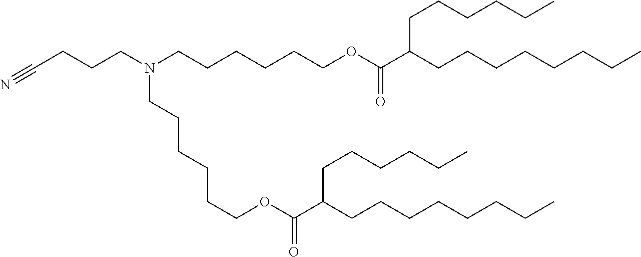 [SMILES: CCCCCCCCC(CCCCCC)C(=O)OCCCCCCN(CCCC#N)CCCCCCOC(=O)C(CCCCCC)CCCCCCCC] |
| I-36 | 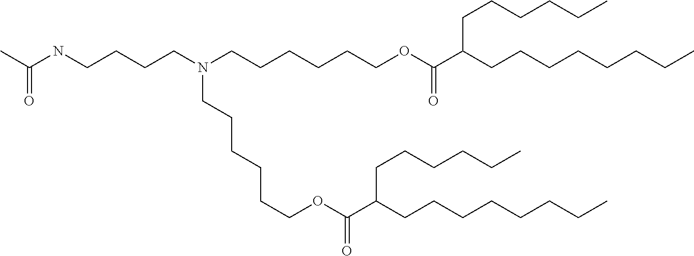 [SMILES: CCCCCCCCC(CCCCCC)C(=O)OCCCCCCN(CCCCCCOC(=O)C(CCCCCC)CCCCCCCC)CCCCNC(C)=O] |
| I-37 | 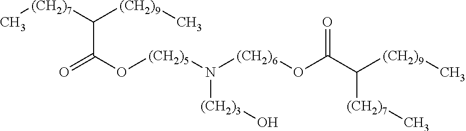 |
| I-38 | 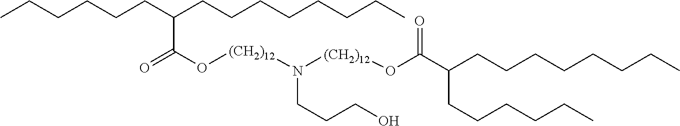 |
| I-39 | 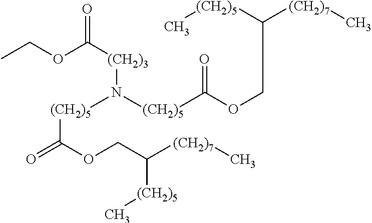 |
| I-40 | 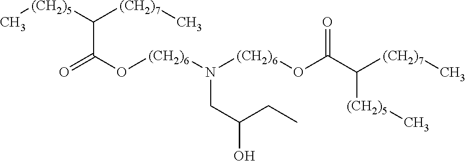 |
| I-41 | 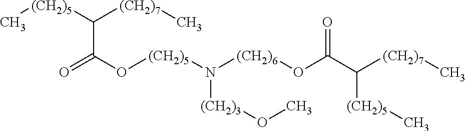 |
| I-42 | 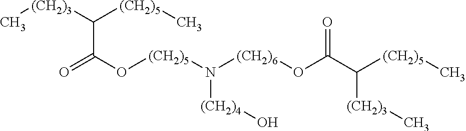 |
| I-43 | 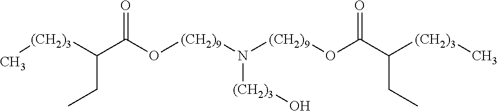 |
| I-44 | 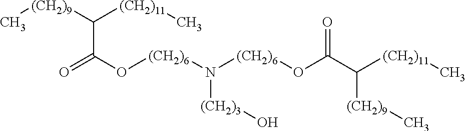 |
| I-45 | 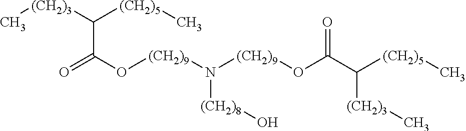 |
| I-46 | 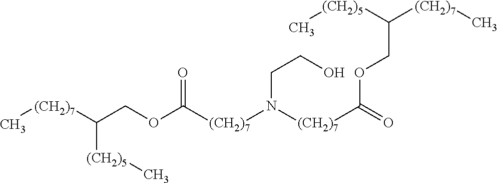 |
| I-47 | 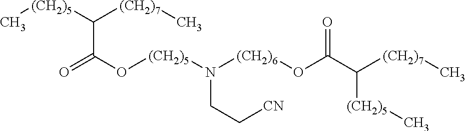 |
| I-48 | 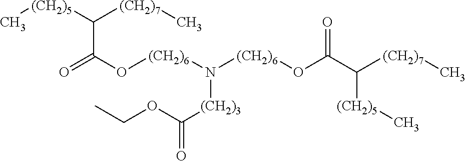 |
| I-49 | 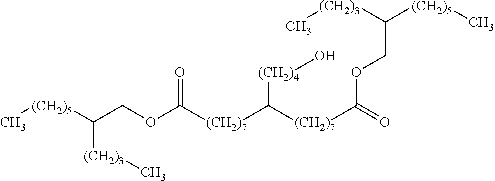 |

or a pharmaceutically acceptable salt thereof. In some embodiments, provided compounds are provided and/or utilized in a salt form (e.g., a pharmaceutically acceptable salt form). Reference to a compound provided herein is understood to include reference to salts thereof, unless otherwise indicated.

In some embodiments, a cationic lipid that may be useful in accordance with the present disclosure is or comprises ((3-hydroxypropyl)azanediyl)bis(nonane-9,1-diyl) bis(2-butyloctanoate) with a chemical structure in Table 5 above as I-45.

In some embodiments, a cationic lipid is selected from DODAC, DOTMA, DDAB, DOTAP, DC-Chol, DMRIE, 1-3, 1-45, and combinations thereof.

In some embodiments, a cationic lipid is I-3. In some embodiments, a cationic lipid is I-45. In some embodiments, a cationic lipid is SM-102. In some embodiments, a cationic lipid is DODAC. In some embodiments, a cationic lipid is DOTMA. In some embodiments, a cationic lipid is DDAB. In some embodiments, a cationic lipid is DOTAP. In some embodiments, a cationic lipid is DC-Chol.

In some embodiments, lipid nanoparticles of the present disclosure comprise about 30 to about 70 mol % of a cationic lipid. In some embodiments, an lipid nanoparticle comprises about 35 to about 65 mol % of a cationic lipid. In some embodiments, an lipid nanoparticle comprises about 40 to about 60 mol % of a cationic lipid. In some embodiments, an lipid nanoparticle comprises about 41 to about 49 mol % of a cationic lipid. In some embodiments, an lipid nanoparticle comprises about 48 mol % of a cationic lipid. In some embodiments, an lipid nanoparticle comprises about 50 mol % of a cationic lipid.

Cationic lipids may be used alone or in combination with neutral lipids, e.g., cholesterol and/or neutral phospholipids, or in combination with other known lipid assembly components.

b. Helper Lipids

As described herein, lipid nanoparticles of the present disclosure comprise one or more helper lipids. In some embodiments, a lipid nanoparticle for delivery of at least one polyribonucleotide described herein comprises one or more helper lipids. A helper lipid may be a neutral lipid, a positively charged lipid, or a negatively charged lipid. In some embodiments, a helper lipid is a lipid that are useful for increasing the effectiveness of delivery of lipid-based particles such as cationic lipid-based particles to a target cell. In some embodiments, a helper lipid may be or comprise a structural lipid with its concentration chosen to optimize lipid nanoparticle particle size, stability, and/or encapsulation.

In some embodiments, a lipid nanoparticle for delivery of polyribonucleotide(s) described herein comprises a neutral helper lipid. Examples of such neutral helper lipids include, but are not limited to phosphotidylcholines such as 1,2-distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-palmitoyl-2-oleoyl-sn-glycero-3-phosphocholine (POPC), 1,2-dioleoyl-sn-glycero-3-phosphocholine (DOPC), phophatidylethanolamines such as 1,2-dioleoyl-sn-glycero-3-phosphoethanolamine (DOPE), sphingomyelins (SM), ceramides, cholesterol, steroids such as sterols and their derivatives. In some embodiments, a steroid is a sterol. In some embodiments, a sterol is cholesterol.

Neutral lipids may be synthetic or naturally derived. Other neutral helper lipids that are known in the art, e.g., as described in WO 2017/075531 and WO 2018/081480, the entire contents of each of which are incorporated herein by reference for the purposes described herein, can also be used in lipid nanoparticles described herein. In some embodiments, a lipid nanoparticle for delivery of polyribonucleotide(s) described herein comprises DSPC and/or cholesterol.

In some embodiments, a lipid nanoparticle described herein comprises multiple neutral lipids (e.g., two neutral lipids). It is understood that reference to “a” neutral lipid is intended to refer to lipid nanoparticles that comprise one or more neutral lipids. In some embodiments, a lipid nanoparticle described herein comprises a phospholipid and/or a steroid. In some embodiments, a lipid nanoparticle described herein comprises DSPC and/or cholesterol.

In some embodiments, a lipid nanoparticle comprises about 5 to about 15 mol % of a phospholipid. In some embodiments, a lipid nanoparticle comprises about 8 to about 12 mol % of a phospholipid. In some embodiments, a lipid nanoparticle comprises about 10 mol % of a phospholipid. In some embodiments, a lipid nanoparticle comprises about 5 to about 15 mol % of DSPC. In some embodiments, a lipid nanoparticle comprises about 8 to about 12 mol % of DSPC. In some embodiments, a lipid nanoparticle comprises about 10 mol % of DSPC.

In some embodiments, a lipid nanoparticle comprises about 30 to about 50 mol % of a steroid. In some embodiments, a lipid nanoparticle comprises about 35 to about 45 mol % of a steroid. In some embodiments, a lipid nanoparticle comprises about 38 to about 40 mol % of a steroid. In some embodiments, a lipid nanoparticle comprises about 38.5 mol % of a steroid. In some embodiments, a lipid nanoparticle comprises about 40 mol % of a steroid.

In some embodiments, a lipid nanoparticle comprises about 30 to about 50 mol % of cholesterol. In some embodiments, a lipid nanoparticle comprises about 35 to about 45 mol % of cholesterol. In some embodiments, a lipid nanoparticle comprises about 38 to about 41 mol % of cholesterol. In some embodiments, a lipid nanoparticle comprises about 38.5 mol % of cholesterol. In some embodiments, a lipid nanoparticle comprises about 40.7 mol % of cholesterol.

In some embodiments, a lipid nanoparticle comprises about 5 to about 15 mol % of phospholipid and about 30 to about 50 mol % of steroid.

In some embodiments, a lipid nanoparticle for delivery of at least one polyribonucleotide described herein comprises at least two helper lipids (e.g., ones described herein). In some such embodiments, a lipid nanoparticle for delivery of at least one polyribonucleotide described herein comprises DSPC and cholesterol.

c. Polymer-Conjugated Lipids

As described herein, lipid nanoparticles of the present disclosure comprise a polymer-conjugated lipid. In some embodiments, a lipid nanoparticle for delivery of at least one polyribonucleotide described herein comprises a polymer-conjugated lipid. A polymer-conjugated lipid is typically a molecule comprising a lipid portion and a polymer portion conjugated thereto.

In some embodiments, a polymer-conjugated lipid is a PEG-conjugated lipid. In some embodiments, a PEG-conjugated lipid is designed to sterically stabilize a lipid particle by forming a protective hydrophilic layer that shields the hydrophobic lipid layer. In some embodiments, a PEG-conjugated lipid can reduce its association with serum proteins and/or the resulting uptake by the reticuloendothelial system when such lipid particles are administered in vivo.

In some embodiments, a PEG lipid is selected from pegylated diacylglycerol (PEG-DAG) such as 1-(monomethoxy-polyethyleneglycol)-2,3-dimyristoylglycerol (PEG-DMG) (e.g., 1,2-dimyristoyl-rac-glycero-3-methoxypolyethylene glycol-2000 (PEG2000-DMG)), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy (polyethoxy)ethyl-N-(2,3-di(tetradecanoxy)propyl)carbamate, and 2,3-di(tetradecanoxy)propy 1-N-(ω methoxy(polyethoxy)ethyl)carbamate.

Certain PEG-conjugated lipids (also known as PEGylated lipids) were clinically approved with safety demonstrated in clinical trials. PEG-conjugated lipids are known to affect cellular uptake, a prerequisite to endosomal localization and payload delivery. The present disclosure, among other things, provides an insight that the pharmacology of encapsulated nucleic acid can be controlled in a predictable manner by modulating the alkyl chain length of a PEG-lipid anchor. In some embodiments, the present disclosure, among other things, provides an insight that such PEG-conjugated lipids may be selected for an polyribonucleotide/lipid nanoparticle drug product formulation to provide optimum delivery of polyribonucleotides to the liver. In some embodiments, such PEG-conjugated lipids may be designed and/or selected based on reasonable solubility characteristics and/or its molecular weight to effectively perform the function of a steric barrier. For example, in some embodiments, such a PEGylated lipid does not show appreciable surfactant or permeability enhancing or disturbing effects on biological membranes. In some embodiments, PEG in such a PEG-conjugated lipid can be linked to diacyl lipid anchors with a biodegradable amide bond, thereby facilitating fast degradation and/or excretion. In some embodiments, a lipid nanoparticle comprising a PEG-conjugated lipid retain a full complement of a PEGylated lipid. In the blood compartment, such a PEGylated lipid dissociates from the particle over time, revealing a more fusogenic particle that is more readily taken up by cells, ultimately leading to release of the RNA payload.

In some embodiments, a PEG-lipid is PEG2000-DMG:

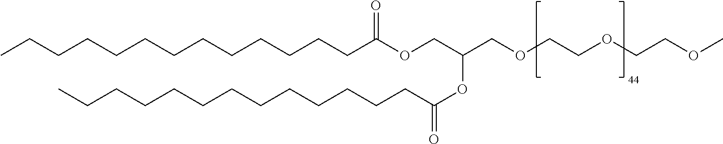

In some embodiments, a lipid nanoparticle may comprise one or more PEG-conjugated lipids or pegylated lipids as described in WO 2017/075531 and WO 2018/081480, the entire contents of each of which are incorporated herein by reference for the purposes described herein. For example, in some embodiments, a PEG-conjugated lipid that may be useful in accordance with the present disclosure can have a structure

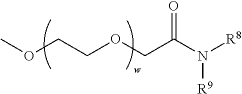

as described in WO 2017/075531, or a pharmaceutically acceptable salt, tautomer or stereoisomer thereof, wherein: R₈ and R₉ are each independently a straight or branched, saturated or unsaturated alkyl chain containing from 10 to 30 carbon atoms, wherein the alkyl chain is optionally interrupted by one or more ester bonds; and w has a mean value ranging from 30 to 60. In some embodiments, R8 and R9 are each independently straight, saturated alkyl chains containing from 12 to 16 carbon atoms. In some embodiments, w has a mean value ranging from 43 to 53. In some embodiments, w is an integer from 40 to 50. In some embodiments, w is 45 to 47. In other embodiments, the average w is about 45. In some embodiments, a PEG-conjugated lipid is or comprises 2-[(Polyethylene glycol)-2000]—N,N-ditetradecylacetamide with a chemical structure as shown as I-3 in Table 5 above and below:

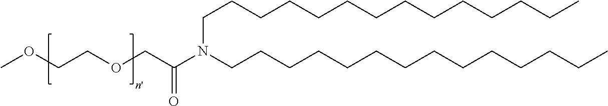

or a pharmaceutically acceptable salt thereof, where n′ is an integer from 45 to 50.

In some embodiments, a PEG-lipid is selected from PEG-DAG, PEG-PE, PEG-S-DAG, PEG2000-DMG, PEG-cer, a PEG dialkyoxypropylcarbamate, ALC-0159, and combinations thereof. In some embodiments, a PEG-lipid is ALC-0159 or PEG2000-DMG. In some embodiments, a PEG-lipid is ALC-0159. In some embodiments, a PEG-lipid is PEG2000-DMG. In some embodiments, a PEG-lipid is PEG-DAG. In some embodiments, a PEG-lipid is PEG-PE. In some embodiments, a PEG-lipid is PEG-S-DAG. In some embodiments, a PEG-lipid is PEG-cer. In some embodiments, a PEG-lipid is a PEG dialkyoxypropylcarbamate.

In some embodiments, a PEG group that is part of a PEG-lipid has, on average in a composition comprising one or more PEG-lipid molecules, a number average molecular weight (Mₙ) of about 2000 g/mol.

In some embodiments, a PEG-lipid is about 0.5 to about 5 mol % relative to total lipids in the lipid nanoparticle. In some embodiments, a lipid nanoparticle comprises about 1.0 to about 2.5 mol % of a PEG-lipid. In some embodiments, a lipid nanoparticle comprises about 1.5 to about 2.0 mol % of a PEG-lipid. In some embodiments, a lipid nanoparticle comprises about 1.5 to about 1.8 mol % of a PEG-lipid.

In some embodiments, a molar ratio of total cationic lipid to total polymer-conjugated lipid (e.g., PEG-conjugated lipid) is from about 100:1 to about 20:1. In some embodiments, a molar ratio of total cationic lipid to total polymer-conjugated lipid (e.g., PEG-conjugated lipid) is from about 50:1 to about 20:1. In some embodiments, a molar ratio of total cationic lipid to total polymer-conjugated lipid (e.g., PEG-conjugated lipid) is from about 40:1 to about 20:1. In some embodiments, a molar ratio of total cationic lipid to total polymer-conjugated lipid (e.g., PEG-conjugated lipid) is from about 35:1 to about 25:1.

In some embodiments, a lipid nanoparticle comprises i) about 30 to about 50 mol % of the cationic lipid; ii) about 1 to about 5 mol % of a PEG-lipid; iii) about 5 to about 15 mol % of a neutral lipid; and iv) about 30 to about 50 mol % of a steroid. In some embodiments, a lipid nanoparticle comprises: i) about 30% to about 50% by weight of ALC-0315; ii) about 1% to about 5% by weight of a ALC-0159; iii) about 5% to about 15% by weight of DSPC; and iv) about 30 to about 50 mol % of cholesterol.

In some embodiments, a lipid nanoparticle comprises: i) about 47.5 mol % of ALC-0315; ii) about 1.8 mol % of a ALC-0159; iii) about 10 mol % of DSPC; and iv) about 40.7 mol % of cholesterol.

In some embodiments, a lipid nanoparticle comprises: i) about 30 to about 50 mol % of SM-102; ii) about 1 to about 5 mol % of a PEG2000-DMG; iii) about 5 to about 15 mol % of DSPC; and iv) about 30 to about 50 mol % of a steroid. In some embodiments, alipid nanoparticle comprises i) about 50 mol % of SM-102; ii) about 1.5 mol % of PEG2000-DMG; iii) about 10 mol % of DSPC; and iv) about 38.5 mol % of cholesterol.

### 3. Exemplary Lipid Nanoparticle Compositions

In some embodiments, lipids that form lipid nanoparticles described herein comprise: a polymer-conjugated lipid; a cationic lipid; and at least one helper lipid. In some such embodiments, total polymer-conjugated lipid may be present in about 0.5-5 mol %, about 0.7-3.5 mol %, about 1-2.5 mol %, about 1.5-2 mol %, or about 1.5-1.8 mol % of the total lipids. In some embodiments, total polymer-conjugated lipid may be present in about 1-2.5 mol % of the total lipids. In some embodiments, the molar ratio of total cationic lipid to total polymer-conjugated lipid (e.g., PEG-conjugated lipid) may be about 100:1 to about 20:1, or about 50:1 to about 20:1, or about 40:1 to about 20:1, or about 35:1 to about 25:1. In some embodiments, the molar ratio of total cationic lipid to total polymer-conjugated lipid may be about 35:1 to about 25:1.

In some embodiments involving a polymer-conjugated lipid, a cationic lipid, and a helper neutral lipid in lipid nanoparticles described herein, total cationic lipid is present in about 35-65 mol %, about 40-60 mol %, about 41-49 mol %, about 41-48 mol %, about 42-48 mol %, about 43-48 mol %, about 44-48 mol %, about 45-48 mol %, about 46-48 mol %, or about 47.2-47.8 mol % of the total lipids. In some embodiments, total cationic lipid is present in about 47.0, 47.1, 47.2, 47.3, 47.4, 47.5, 47.6, 47.7, 47.8, 47.9 or 48.0 mol % of the total lipids.

In some embodiments involving a polymer-conjugated lipid, a cationic lipid, and a helper neutral lipid in lipid nanoparticles described herein, total neutral lipid is present in about 35-65 mol %, about 40-60 mol %, about 45-55 mol %, or about 47-52 mol % of the total lipids. In some embodiments, total neutral lipid is present in 35-65 mol % of the total lipids. In some embodiments, total non-steroid neutral lipid (e.g., DPSC) is present in about 5-15 mol %, about 7-13 mol %, or 9-11 mol % of the total lipids. In some embodiments, total non-steroid neutral lipid is present in about 9.5, 10 or 10.5 mol % of the total lipids. In some embodiments, the molar ratio of the total cationic lipid to the non-steroid neutral lipid ranges from about 4.1:1.0 to about 4.9:1.0, from about 4.5:1.0 to about 4.8:1.0, or from about 4.7:1.0 to 4.8:1.0. In some embodiments, total steroid neutral lipid (e.g., cholesterol) is present in about 35-50 mol %, about 39-49 mol %, about 40-46 mol %, about 40-44 mol %, or about 40-42 mol % of the total lipids. In some embodiments, total steroid neutral lipid (e.g., cholesterol) is present in about 39, 40, 41, 42, 43, 44, 45, or 46 mol % of the total lipids. In some embodiments, the molar ratio of total cationic lipid to total steroid neutral lipid is about 1.5:1 to 1:1.2, or about 1.2:1 to 1:1.2.

In some embodiments, a lipid composition comprising a cationic lipid, a polymer-conjugated lipid, and a neutral lipid can have individual lipids present in certain molar percents of the total lipids, or in certain molar ratios (relative to each other) as described in WO 2018/081480, the entire contents of each of which are incorporated herein by reference for the purposes described herein.

In some embodiments, lipids that form the lipid nanoparticles comprise: a polymer-conjugated lipid (e.g., PEG-conjugated lipid); a cationic lipid; and a neutral lipid, wherein the polymer-conjugated lipid is present in about 1-2.5 mol % of the total lipids; the cationic lipid is present in 35-65 mol % of the total lipids; and the neutral lipid is present in 35-65 mol % of the total lipids. In some embodiments, lipids that form the lipid nanoparticles comprise: a polymer-conjugated lipid (e.g., PEG-conjugated lipid); a cationic lipid; and a neutral lipid, wherein the polymer-conjugated lipid is present in about 1-2 mol % of the total lipids; the cationic lipid is present in 45-48.5 mol % of the total lipids; and the neutral lipid is present in 45-55 mol % of the total lipids. In some embodiments, lipids that form the lipid nanoparticles comprise: a polymer-conjugated lipid (e.g., PEG-conjugated lipid); a cationic lipid; and a neutral lipid comprising a non-steroid neutral lipid and a steroid neutral lipid, wherein the polymer-conjugated lipid is present in about 1-2 mol % of the total lipids; the cationic lipid is present in 45-48.5 mol % of the total lipids; the non-steroid neutral lipid is present in 9-11 mol % of the total lipids; and the steroid neutral lipid is present in about 36-44 mol % of the total lipids. In many of such embodiments, a PEG-conjugated lipid is or comprises 2-[(polyethylene glycol)-2000]—N,N-ditetradecylacetamide or a derivative thereof. In many of such embodiments, a cationic lid is or comprises ((3-hydroxypropyl)azanediyl)bis(nonane-9,1-diyl)bis(2-butyloctanoate) or a derivative thereof. In many of such embodiments, a neutral lipid comprises DSPC and cholesterol, wherein DSPC is a non-steroid neutral lipid and cholesterol is a steroid neutral lipid.

In some embodiments, lipids that form the lipid nanoparticles comprise:
(a) 2-[(polyethylene glycol)-2000]—N,N-ditetradecylacetamide at about 1-2.5 mol % of the total lipids;
(b) DPSC and cholesterol, wherein together DPSC and cholesterol are about 35-65 mol % of the total lipids; and
(c) ((3-hydroxypropyl)azanediyl)bis(nonane-9,1-diyl)bis(2-butyloctanoate) at about 35-65 mol % of the total lipids.

### B. Exemplary Methods of Making Lipid Nanoparticles

Lipids and lipid nanoparticles comprising nucleic acids and their method of preparation are known in the art, including, e.g., as described in U.S. Pat. Nos. 8,569,256, 5,965,542 and U.S. Patent Publication Nos. 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583, 2011/0311582, 2011/0262527, 2011/0216622, 2011/0117125, 2011/0091525, 2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos. WO 99/39741, WO 2018/081480, WO 2017/004143, WO 2017/075531, WO 2015/199952, WO 2014/008334, WO 2013/086373, WO 2013/086322, WO 2013/016058, WO 2013/086373, WO2011/141705, and WO 2001/07548, the full disclosures of which are herein incorporated by reference in their entirety for the purposes described herein.

For example, in some embodiments, cationic lipids, neutral lipids (e.g., DSPC, and/or cholesterol) and polymer-conjugated lipids can be solubilized in ethanol at a predetermined molar ratio (e.g., ones described herein). In some embodiments, lipid nanoparticles (lipid nanoparticle) are prepared at a total lipid to polyribonucleotides weight ratio of approximately 10:1 to 30:1. In some embodiments, such polyribonucleotides can be diluted to 0.2 mg/mL in acetate buffer.

In some embodiments, using an ethanol injection technique, a colloidal lipid dispersion comprising polyribonucleotides can be formed as follows: an ethanol solution comprising lipids, such as cationic lipids, neutral lipids, and polymer-conjugated lipids, is injected into an aqueous solution comprising polyribonucleotides (e.g., ones described herein).

In some embodiments, lipid and polyribonucleotide solutions can be mixed at room temperature by pumping each solution at controlled flow rates into a mixing unit, for example, using piston pumps. In some embodiments, the flow rates of a lipid solution and a RNA solution into a mixing unit are maintained at a ratio of 1:3. Upon mixing, nucleic acid-lipid particles are formed as the ethanolic lipid solution is diluted with aqueous polyribonucleotides. The lipid solubility is decreased, while cationic lipids bearing a positive charge interact with the negatively charged RNA.

In some embodiments, a solution comprising RNA-encapsulated lipid nanoparticles can be processed by one or more of concentration adjustment, buffer exchange, formulation, and/or filtration.

In some embodiments, RNA-encapsulated lipid nanoparticles can be processed through filtration.

In some embodiments, particle size and/or internal structure of lipid nanoparticles (with or with ssRNs) may be monitored by appropriate techniques such as, e.g., small-angle X-ray scattering (SAXS) and/or transmission electron cryomicroscopy (CryoTEM).

### IV. Exemplary Compositions

The present disclosure provides vaccine compositions, including lipid nanoparticles (LNPs) as described herein that incorporate at least one (e.g., one, 2 or more, 3 or more, 4 or more, 5 or more, or 6 or more) polyribonucleotide, where the at least one polyribonucleotide encodes at least one (e.g., one, 2 or more, 3 or more, 4 or more, 5 or more, or 6 or more) MPXV antigen.

In some embodiments, a vaccine composition of the present disclosure includes a LNP incorporating two polyribonucleotides. In some embodiments, a vaccine composition includes a LNP incorporating: (i) a polyribonucleotide encoding a B6R antigen, or fragment thereof, having an amino acid sequence at least 85% identical (e.g., at least 85%, at least 90%, at least 91%, at least 92%, at least 93%, at least 94%, at least 95%, at least 96%, at least 97%, at least 98%, at least 99%, or 100%) to any one of SEQ ID NOs: 21-30, 180, and 182; and (ii) a polyribonucleotide encoding a M1R antigen, or fragment thereof, having an amino acid sequence at least 85% identical (e.g., at least 85%, at least 90%, at least 91%, at least 92%, at least 93%, at least 94%, at least 95%, at least 96%, at least 97%, at least 98%, at least 99%, or 100%) to any one of SEQ ID NOs: 31-40, and 158.

In some embodiments, a vaccine composition of the present disclosure includes a LNP incorporating three polyribonucleotides. In some embodiments, a vaccine composition includes a LNP incorporating: (i) a polyribonucleotide encoding a B6R antigen, or fragment thereof, having an amino acid sequence at least 85% identical (e.g., at least 85%, at least 90%, at least 91%, at least 92%, at least 93%, at least 94%, at least 95%, at least 96%, at least 97%, at least 98%, at least 99%, or 100%) to any one of SEQ ID NOs: 21-30, 180 and 189; (ii) a polyribonucleotide encoding a M1R antigen, or fragment thereof, having an amino acid sequence at least 85% identical (e.g., at least 85%, at least 90%, at least 91%, at least 92%, at least 93%, at least 94%, at least 95%, at least 96%, at least 97%, at least 98%, at least 99%, or 100%) to any one of SEQ ID NOs: 31-40, and 158; and (iii) a polyribonucleotide encoding an A35R antigen, or fragment thereof, having an amino acid sequence at least 85% identical (e.g., at least 85%, at least 90%, at least 91%, at least 92%, at least 93%, at least 94%, at least 95%, at least 96%, at least 97%, at least 98%, at least 99%, or 100%) to any one of SEQ ID NOs: 11-20, 179 and 174.

In some embodiments, a vaccine composition of the present disclosure includes a LNP incorporating four polyribonucleotides. In some embodiments, a vaccine composition includes a LNP incorporating: (i) a polyribonucleotide encoding a B6R antigen, or fragment thereof, having an amino acid sequence at least 85% identical (e.g., at least 85%, at least 90%, at least 91%, at least 92%, at least 93%, at least 94%, at least 95%, at least 96%, at least 97%, at least 98%, at least 99%, or 100%) to any one of SEQ ID NOs: 21-30, 180, and 189; (ii) a polyribonucleotide encoding a M1R antigen, or fragment thereof, having an amino acid sequence at least 85% identical (e.g., at least 85%, at least 90%, at least 91%, at least 92%, at least 93%, at least 94%, at least 95%, at least 96%, at least 97%, at least 98%, at least 99%, or 100%) to any one of SEQ ID NOs: 31-40, and 158; (iii) a polyribonucleotide encoding an A35R antigen, or fragment thereof, having an amino acid sequence at least 85% identical (e.g., at least 85%, at least 90%, at least 91%, at least 92%, at least 93%, at least 94%, at least 95%, at least 96%, at least 97%, at least 98%, at least 99%, or 100%) to any one of SEQ ID NOs: 11-20, 172, and 174; and (iv) a polyribonucleotide encoding an E8L antigen, or fragment thereof, having an amino acid sequence at least 85% identical (e.g., at least 85%, at least 90%, at least 91%, at least 92%, at least 93%, at least 94%, at least 95%, at least 96%, at least 97%, at least 98%, at least 99%, or 100%) to any one of SEQ ID NOs: 41-50, and 192.

In some embodiments, a vaccine composition of the present disclosure includes a LNP incorporating four polyribonucleotides. In some embodiments, a vaccine composition includes a LNP incorporating: (i) a polyribonucleotide encoding a B6R antigen, or fragment thereof, having an amino acid sequence at least 85% identical (e.g., at least 85%, at least 90%, at least 91%, at least 92%, at least 93%, at least 94%, at least 95%, at least 96%, at least 97%, at least 98%, at least 99%, or 100%) to any one of SEQ ID NOs: 21-30, 180, and 182; (ii) a polyribonucleotide encoding a M1R antigen, or fragment thereof, having an amino acid sequence at least 85% identical (e.g., at least 85%, at least 90%, at least 91%, at least 92%, at least 93%, at least 94%, at least 95%, at least 96%, at least 97%, at least 98%, at least 99%, or 100%) to any one of SEQ ID NOs: 31-40, and 158; (iii) a polyribonucleotide encoding an A35R antigen, or fragment thereof, having an amino acid sequence at least 85% identical (e.g., at least 85%, at least 90%, at least 91%, at least 92%, at least 93%, at least 94%, at least 95%, at least 96%, at least 97%, at least 98%, at least 99%, or 100%) to any one of SEQ ID NOs: 11-20, 172, and 174; and (iv) a polyribonucleotide encoding an H3L antigen, or fragment thereof, having an amino acid sequence at least 85% identical (e.g., at least 85%, at least 90%, at least 91%, at least 92%, at least 93%, at least 94%, at least 95%, at least 96%, at least 97%, at least 98%, at least 99%, or 100%) to any one of SEQ ID NOs: 51-60, 184, 188, and 190.

### V. Pharmaceutical Compositions

The present disclosure provides compositions, e.g., pharmaceutical compositions comprising one or more polyribonucleotides described herein. Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, an excipient is approved for use in humans and for veterinary use. In some embodiments, an excipient is approved by the United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

General considerations in the formulation and/or manufacture of pharmaceutical agents may be found, for example, in Remington: The Science and Practice of Pharmacy 21st ed., Lippincott Williams & Wilkins, 2005 (incorporated herein by reference).

In some embodiments, pharmaceutical compositions provided herein may be formulated with one or more pharmaceutically acceptable carriers or diluents as well as any other known adjuvants and excipients in accordance with conventional techniques such as those disclosed in Remington: The Science and Practice of Pharmacy 21st ed., Lippincott Williams & Wilkins, 2005 (incorporated herein by reference).

Pharmaceutical compositions described herein can be administered by appropriate methods known in the art. As will be appreciated by a skilled artisan, the route and/or mode of administration may depend on a number of factors, including, e.g., but not limited to stability and/or pharmacokinetics and/or pharmacodynamics of pharmaceutical compositions described herein.

In some embodiments, pharmaceutical compositions described herein are formulated for parenteral administration, which includes modes of administration other than enteral and topical administration, usually by injection, and includes, without limitation, intravenous, intramuscular, intraarterial, intradermal, subcutaneous, subcuticular, or intraarticular injection and infusion. In preferred embodiments, pharmaceutical compositions described herein are formulated for intravenous, intramuscular, or subcutaneous administration.

In some embodiments, pharmaceutical compositions described herein are formulated for intravenous administration. In some embodiments, pharmaceutically acceptable excipients that may be useful for intravenous administration include sterile aqueous solutions or dispersions and sterile powders for preparation of sterile injectable solutions or dispersions.

Therapeutic compositions typically must be sterile and stable under the conditions of manufacture and storage. The composition can be formulated as a solution, microemulsion, lipid nanoparticles, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. Proper fluidity can be maintained, for example, by the use of surfactants. In many cases, it will be preferable to include isotonic agents, for example, sugars, polyalcohols such as mannitol, sorbitol, or sodium chloride in the composition. In some embodiments, prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization and/or microfiltration. In some embodiments, pharmaceutical compositions can be prepared as described herein and/or methods known in the art.

These compositions may also contain adjuvants such as preservatives, wetting agents, emulsifying agents and dispersing agents. Prevention of the presence of microorganisms may be ensured both by sterilization procedures, and by the inclusion of various antibacterial and antifungal agents, for example, paraben, chlorobutanol, phenol sorbic acid, and the like. It may also be desirable to include isotonic agents, such as sugars, sodium chloride, and the like into pharmaceutical compositions described herein. In addition, prolonged absorption of the injectable pharmaceutical form may be brought about by the inclusion of agents which delay absorption such as aluminum monostearate and gelatin.

Formulations of pharmaceutical compositions described herein may be prepared by any method known or hereafter developed in the art of pharmacology. In general, such preparatory methods include the step of bringing active ingredient(s) into association with a diluent or another excipient and/or one or more other accessory ingredients, and then, if necessary and/or desirable, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of at least one RNA product produced using a system and/or method described herein.

Relative amounts of polyribonucleotides encapsulated in lipid nanoparticles, a pharmaceutically acceptable excipient, and/or any additional ingredients in a pharmaceutical composition can vary, depending upon the subject to be treated, target cells, diseases or disorders, and may also further depend upon the route by which the composition is to be administered.

In some embodiments, pharmaceutical compositions described herein are formulated into pharmaceutically acceptable dosage forms by conventional methods known to those of skill in the art. Actual dosage levels of the active ingredients (e.g., polyribonucleotides encapsulated in lipid nanoparticles) in the pharmaceutical compositions described herein may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A physician having ordinary skill in the art can readily determine and prescribe the effective amount of the pharmaceutical composition required. For example, a physician could start doses of active ingredients (e.g., polyribonucleotides encapsulated in lipid nanoparticles) employed in the pharmaceutical composition at levels lower than that required in order to achieve the desired therapeutic effect and gradually increase the dosage until the desired effect is achieved.

In some embodiments, a pharmaceutical composition described herein is formulated (e.g., but not limited to, for intravenous, intramuscular, or subcutaneous administration) to deliver an active dose that confers a plasma concentration of an antigen or fragment thereof encoded by at least one polyribonucleotide (e.g., ones described herein) that mediates pharmacological activity via its dominant mode of action, viral neutralization.

In some embodiments, a pharmaceutical composition is formulated (e.g., but not limited to, for intravenous, intramuscular, or subcutaneous administration) to deliver a dose of 5 mg RNA/kg.

In some embodiments, a pharmaceutical composition described herein may further comprise one or more additives, for example, in some embodiments that may enhance stability of such a composition under certain conditions. Examples of additives may include but are not limited to salts, buffer substances, preservatives, and carriers. For example, in some embodiments, a pharmaceutical composition may further comprise a cryoprotectant (e.g., sucrose) and/or an aqueous buffered solution, which may in some embodiments include one or more salts, including, e.g., alkali metal salts or alkaline earth metal salts such as, e.g., sodium salts, potassium salts, and/or calcium salts.

In some embodiments, a pharmaceutical composition provided herein is a preservative-free, sterile RNA-lipid nanoparticle dispersion in an aqueous buffer for intravenous or intramuscular administration.

Although the descriptions of pharmaceutical compositions provided herein are principally directed to pharmaceutical compositions that are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation.

### VI. Characterization

Without wishing to be bound by any particular theory, it is proposed that ability to induce an antibody response may be important to effectiveness of a composition for treatment and/or prevention of orthopox (e.g., monkeypox) infection, (e.g., a pharmaceutical composition, an immunogenic composition, or a vaccine).

In some embodiments, provided technologies (e.g., compositions and/or dosing regimens, etc) are characterized by an ability to induce (e.g., when administered to a model system and/or to a human, for example by parenteral administration such as by intramuscular administration) an antibody response targeting one or more monkeypox antigen(s) described herein. That is, in some embodiments, provided technologies are characterized in that, when administered (e.g., by parenteral administration such as by intramuscular administration) to an organism (e.g., a model organism or an animal or human organism in need of protection), provided technologies induce a robust antibody response targeting one or more monkeypox antigens.

In some embodiments, provided technologies are characterized in that they induce antibody titers to a level that provides sufficient protective response against an orthopox virus (e.g., monkeypox), when administered to a relevant population.

In some embodiments, provided technologies are characterized in that they induce antibody titers to one or more monkeypox antigen(s) in a range of 10\hat{ }3-10\hat{ }6 after at 5 days, 10 days, 21 days, or 28 days post immunization.

In some embodiments, administration of a composition described herein comprising one or more polyribonucleotides each encoding one or more monkeypox antigens or fragments thereof is characterized in that it can induce antibody titers of at least 10\hat{ }3 by 5 days post immunization. In some embodiments, administration of a composition described herein comprising one or more polyribonucleotides each encoding one or more monkeypox antigens or fragments thereof is characterized in that it can induce antibody titers of at least 10\hat{ }4 by 10 days post immunization. In some embodiments, administration of a composition described herein comprising one or more polyribonucleotides each encoding one or more monkeypox antigens or fragments thereof is characterized in that it can induce antibody titers of at least 10\hat{ }5 by 21 days post immunization.

In some embodiments, administration of a second dose of a composition described herein induces a further increase in antibody titers. In some embodiments, a second dose is administered at least 21 days after a first dose. In some embodiments, a second dose is characterized in that it can induce antibody titers of at least 10\hat{ }6 by at least 5 days after administration of the second dose. In some embodiments, a second dose is characterized in that it can induce antibody titers of at least 10\hat{ }6 by at least 10 days after administration of the second dose.

In some embodiments, administration of a composition described herein that delivers two or more monkeypox antigens in combination induces similar antibody levels to single antigen immunization.

In some embodiments, administration of a composition described herein that delivers two or more monkeypox antigens is characterized in that it induces an antibody response in a range of 10\hat{ }3-10\hat{ }6 after at 5 days, 10 days, 21 days, or 28 days post immunization.

In some embodiments, administration of a composition described herein that delivers two or more monkeypox antigens is characterized in that it induces antibody titers of at least 10\hat{ }3 by 5 days post immunization. In some embodiments, administration of a composition described herein that delivers two or more monkeypox antigens is characterized in that it induces antibody titers of at least 10\hat{ }4 by 10 days post immunization. In some embodiments, administration of a composition described herein that delivers two or more monkeypox antigens is characterized in that it induces antibody titers of at least 10\hat{ }5 by 21 days post immunization.

In some embodiments, administration of a composition described herein to a subject induces antibody production (e.g., IgG, IgA, IgM, IgE). For example, in some embodiments, administration of a composition described herein to a subject induces production of one or more of IgG1, IgG2A, IgG2B, and IgG3.

### VII. Patient Populations

Technologies provided herein can be useful for treatment and/or prevention of an orthopox (e.g., monkeypox) infection. As described herein, technologies include polyribonucleotides. Accordingly, the present disclosure provides pharmaceutical compositions for treatment and. or prevention of monkeypox. In some embodiments, a pharmaceutical composition comprises a polyribonucleotides as described herein.

In some embodiments, a subject is one suffering from and/or is susceptible to an orthopox infection. In some embodiments, a subject is one suffering from and/or is susceptible to monkeypox infection. In some embodiments, a subject is one suffering from and/or is susceptible to variola infection. In some embodiments, a subject is one suffering from and/or is susceptible to vaccinia infection. In some embodiments, a subject may be defined by one or more criterion such as age group, gender, genetic background, preexisting clinical conditions, and/or prior exposure to therapy.

In some embodiments, a subject is a model organism. In preferred embodiments, a subject is a human. In some embodiments, a subject is between 18-65 years of age. In some embodiments, a subject is an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old, or from about 95 to about 100 years old.

In some embodiments, a subject is a human infant. In some embodiments, a subject is a human toddler. In some embodiments, a subject is a human child. In some embodiments, a subject is a human adult. In some embodiments, a subject is an elderly human.

Among the various advantages of certain methods and compositions provided herein, including in particular methods and compositions of monkeypox vaccination that include administration of polyribonucleotide of the present disclosure, are that such methods and compositions are not subject to certain limitations that characterize other vaccine technologies. To provide one particular example, live attenuated virus vaccines (such as Dryvax) are contraindicated for subjects who are immunocompromised (e.g., severely immunocompromised) at least in part because such subjects are at increased risk for serious adverse reactions. Subjects with most forms of altered immunocompetence should not receive live vaccines. The present disclosure includes that methods and compositions provided herein can be used to treat (e.g., to vaccinate) subjects who are immunocompromised (e.g., severely immunocompromised) and/or subjects with altered immunocomptence (including without limitation primary and/or secondary immunosuppression, immunodeficiency, and immunocompromised).

### VIII. Treatment Methods

In some embodiments, a pharmaceutical composition described herein can be taken up by cells for production of an encoded agent at therapeutically relevant serum concentrations. Accordingly, the present disclosure provides methods of using pharmaceutical compositions described herein. For example, in some embodiments, a method provided herein comprises administering a pharmaceutical composition described herein to a subject.

As used herein, the term “administering” or “administration” typically refers to the administration of a composition to a subject to achieve delivery of an agent (e.g., at least one polyribonucleotide encoding an antigen or fragment thereof described herein) that is, or is included in, a composition to a target site or a site to be treated. Those of ordinary skill in the art will be aware of a variety of routes that may, in appropriate circumstances, be utilized for administration to a subject, for example a human. Administration may be, for example, bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In preferred embodiments, administration may be intramuscular, intravenous, or subcutaneous.

In some embodiments, administration of a pharmaceutical composition results in delivery of one or more polyribonucleotides as described herein (e.g., encoding an antigen or fragment thereof) to a subject. In some embodiments, administering a pharmaceutical composition to a subject results in expression in the subject of an antigen or fragment thereof encoded by an administered polyribonucleotide. In some embodiments, administering a pharmaceutical composition to a subject results in expression in the subject of an antigen or fragment thereof encoded by an administered polyribonucleotide.

In some embodiments, a pharmaceutical composition for administration to a subject is provided as two or more separate particle compositions each comprising one or more polyribonucleotides of the present disclosure (e.g., encoding a monkeypox antigen as described herein), which are then mixed together prior to administration. For example, in some embodiments, individual populations of nucleic acid containing particles, each population comprising an RNA molecule encoding a different immunogenic polypeptide or immunogenic fragment thereof (e.g., a monkeypox antigen as described herein), can be separately formed and then mixed together, for example, prior to filling into vials during a manufacturing process, or immediately prior to administration). Accordingly, in some embodiments, described herein is a composition comprises two or more populations of particles (e.g., in some embodiments, lipid nanoparticles), each population comprising at least one RNA molecule encoding a different immunogenic polypeptide or immunogenic fragment thereof (e.g., monkeypox antigen or fragment thereof). In some embodiments, each population may be provided in a composition at a desirable proportion (e.g., in some embodiments, each population may be provided in a composition in an amount that provides the same amount of RNA molecules).

In some embodiments, administered pharmaceutical compositions (e.g., immunogenic compositions, e.g., vaccines) comprising polyribonucleotides that encode one or more monkeypox antigens (e.g., from Table 1 or Table 2 or fragments thereof) are administered in RNA doses of from about 0.1 μg to about 300 μg, about 0.5 μg to about 200 μg, or about 1 μg to about 100 μg, such as about 1 μg, about 3 μg, about 10 μg, about 30 μg, about 50 μg, or about 100 μg.

In some embodiments, a pharmaceutical composition comprising a polyribonucleotide that encodes one or more monkeypox antigens (e.g., from Table 1 or Table 2 or fragments thereof) are administered at RNA doses of from about 0.5 μg to about 10 μg. In some embodiments, a pharmaceutical composition comprises one polyribonucleotide encoding a monkeypox antigen that is administered at a dose of from about 0.5 μg to about 2 μg. In some embodiments, a pharmaceutical composition comprises one polyribonucleotide encoding a monkeypox antigen (e.g., from Table 1 or Table 2 or a fragment thereof) that is administered at a dose of about 1 μg. In some embodiments, a pharmaceutical composition comprises one polyribonucleotide encoding one, two, or more monkeypox antigens, wherein each polyribonucleotide is administered at a dose of from about 0.5 μg to about 2 μg. In some embodiments, a pharmaceutical composition comprises one polyribonucleotide encoding one, two, or more monkeypox antigens, wherein the polyribonucleotide is administered at a dose of about 1 μg. In some embodiments, the one, two or more monkeypox antigens are a B cell antigen from Table 1 or a fragment thereof. In some embodiments, the one, two or more monkeypox antigens are a T cell antigen from Table 2 or a fragment thereof.

In some embodiments, a pharmaceutical composition comprises two or more polyribonucleotides each encoding a monkeypox antigen (e.g., from Table 1 or Table 2 or a fragment thereof), wherein each RNA construct is administered at a dose of from about 0.5 μg to about 2 μg. In some embodiments, a pharmaceutical composition comprises two or more polyribonucleotide each encoding a monkeypox antigen (e.g., from Table 1 or Table 2 or a fragment thereof), wherein each RNA construct is administered at a dose of about 1 μg.

In some embodiments, one or more pharmaceutical compositions are administered, comprising two or more RNA monkeypox constructs (e.g., two, three, four, five, six or more RNA constructs), wherein each RNA construct is administered at a dose of about 1 μg. In some embodiments, two or more pharmaceutical compositions are administered, together comprising two or more RNA constructs each encoding one or more monkeypox antigens (e.g., from Table 1 or Table 2 or a fragment thereof), wherein each RNA construct is administered at a dose of about 1 μg. In some embodiments, two or more pharmaceutical compositions are administered, together comprising two, three, four, five, six or more polyribonucleotides encoding monkeypox antigens, wherein each polyribonucleotide is administered at a dose of about 1 μg. In some embodiments, one or more pharmaceutical compositions are administered, together comprising one or more polyribonucleotides encoding two to four monkeypox antigens (e.g., from Table 1 or Table 2 or a fragment thereof), wherein each polyribonucleotide is administered at a dose of about 1 μg. In some embodiments, one or more pharmaceutical compositions are administered, together comprising one or more polyribonucleotides encoding two to four monkeypox B cell antigens (e.g., from Table 1 or a fragment thereof), wherein each polyribonucleotide is administered at a dose of about 1 μg.

In some embodiments, one or more pharmaceutical compositions are administered, together comprising one or more polyribonucleotides encoding two to four monkeypox B cell antigens selected from E8L, A35R, B6R, M1R, H3L, A28L, A29L, and/or fragments of any thereof, wherein each polyribonucleotide is administered at a dose of about 1 μg. In some embodiments, one or more pharmaceutical compositions are administered, together comprising one or more polyribonucleotides encoding two to four monkeypox B cell antigens selected from B6R, A35R, M1R, H3L, E8L, and/or fragments of any thereof. In some embodiments, one or more pharmaceutical compositions are administered, together comprising one or more polyribonucleotides encoding four monkeypox B cell antigens that are B6R, A35R, M1R, and H3L, (or fragments of any thereof). In some embodiments, one or more pharmaceutical compositions are administered, together comprising one or more polyribonucleotides encoding four monkeypox B cell antigens that are B6R, A35R, M1R, E8L (or fragments of any thereof). In some embodiments, one or more pharmaceutical compositions are administered, together comprising one or more polyribonucleotides encoding three monkeypox B cell antigens that are B6R, A35R, and M1R, (or fragments of any thereof).

In some embodiments, a pharmaceutical composition is administered, comprising two polyribonucleotides each encoding one or more monkeypox antigens, wherein a total polyribonucleotide dose administered is about 2 to 4 μg. In some embodiments, two or more pharmaceutical compositions are administered, together comprising two polyribonucleotides each encoding one or more monkeypox antigens, wherein a total polyribonucleotide dose administered is about 2 to 4 μg.

In some embodiments, a pharmaceutical composition is administered, comprising one or more polyribonucleotides encoding one or more monkeypox antigens, wherein a total polyribonucleotide dose administered is about 10 μg, 30 μg, or 60 μg. In some embodiments, a pharmaceutical composition is administered, comprising two polyribonucleotides each encoding one or more monkeypox antigens, wherein a total polyribonucleotide dose administered is about 10 μg, 30 μg, or 60 μg. In some embodiments, two or more pharmaceutical compositions are administered, together comprising two polyribonucleotides each encoding one or more monkeypox antigens, wherein a total polyribonucleotide dose administered is about 10 μg, 30 μg, or 60 μg. In some embodiments, the one or more monkeypox antigens are B cell antigens selected from E8L, A35R, B6R, M1R, H3L, A28L, A29L, and/or fragments of any thereof. In some embodiments, wherein the one or more monkeypox antigens are two to four monkeypox B cell antigens selected from B6R, A35R, M1R, H3L, E8L, and/or fragments of any thereof.

In some embodiments, administration may involve only a single dose. In some embodiments, administration may involve application of a fixed number of doses.

In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Those skilled in the art are aware that therapies can be administered in dosing cycles. In some embodiments, pharmaceutical compositions described herein are administered in one or more dosing cycles.

In some embodiments, one dosing cycle is at least 3 or more days (including, e.g., at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 days. In some embodiments, one dosing cycle is at least 21 days.

In some embodiments, one dosing cycle may involve multiple doses, e.g., according to a pattern such as, for example, a dose may be administered daily within a dosing cycle, or a dose may be administered every 2 days, every 3 days, every 4 days, every 5 days, every 6 days, every 7 days, every 2 weeks, monthly, every 2 months within a cycle. In some certain embodiments, a dosing cycle is at least 4 weeks. In some certain embodiments, a dosing cycle is about 4 weeks.

In some embodiments, multiple dosing cycles may be administered. For example, in some embodiments, at least 2 dosing cycles (including, e.g., at least 3 dosing cycles, at least 4 dosing cycles, at least 5 dosing cycles, at least 6 dosing cycles, at least 7 dosing cycles, at least 8 dosing cycles, at least 9 dosing cycles, at least 10 dosing cycles, or more) can be administered. In some embodiments, the number of dosing cycles to be administered may vary with types of treatment (e.g., monotherapy vs. combination therapy). In some embodiments, at least 3-8 dosing cycles may be administered.

In some embodiments, there may be a “rest period” between dosing cycles; in some embodiments, there may be no rest period between dosing cycles. In some embodiments, there may be sometimes a rest period and sometimes no rest period between dosing cycles.

In some embodiments, a rest period may have a length within a range of several days to several months. For example, in some embodiments, a rest period may have a length of at least 3 days or more, including, e.g., at least 4 days, at least 5 days, at least 6 days, at least 7 days, at least 8 days, at least 9 days, at least 10 days, at least 11 days, at least 12 days, at least 13 days, at least 14 days or more. In some embodiments, a rest period may have a length of at least 1 week or more, including, e.g., at least 2 weeks, at least 3 weeks, at least 4 weeks, or more. In certain embodiments, a rest period is at least 4 weeks. In certain embodiments, a rest period is about 4 weeks.

Dosage of pharmaceutical compositions described herein may vary with a number of factors including, e.g., but not limited to body weight of a subject to be treated, cancer types and/or cancer stages, and/or monotherapy or combination therapy. In some embodiments, a dosing cycle involves administration of a set number and/or pattern of doses. For example, in some embodiments, a pharmaceutical composition described herein is administered at least one dose per dosing cycle, including, e.g., at least two doses per dosing cycle, at least three doses per dosing cycle, at least four doses per dosing cycle, or more.

In some embodiments, a dosing cycle involves administration of a set cumulative dose, e.g., over a particular period of time, and optionally via multiple doses, which may be administered, for example, at set interval(s) and/or according to a set pattern. In some embodiments, a set cumulative dose may be administered via multiple doses at set intervals such that there is at least some temporal overlap in biological and/or pharmacokinetics effects generated by such multiple doses on a target cell or on a subject being treated. In some embodiments, a set cumulative dose may be administered via multiple doses at set intervals such that biological and/or pharmacokinetics effects generated by such multiple doses on a target cell or on a subject being treated may be additive. By way of example only, in some embodiments, a set cumulative dose of X mg may be administered via two doses with each dose of X/2 mg, wherein such two doses are administered sufficiently close in time such that biological and/or pharmacokinetics effects generated by each X/2-mg dose on a target cell or on a subject being treated may be additive.

In some embodiments, dosing may be adjusted based on response of a subject receiving the therapy. For example, in some embodiments, dosing may involve administration of a higher dose followed later by administration of a lower dose if one or more parameters for safety pharmacology assessment indicates that the prior dose may not satisfy the medical safety requirement according to a physician. In some embodiments, dose escalation may be performed at one or more of the levels. Without wishing to be bound by any particular theory, the present disclosure, among other things, provides an insight that a pharmaceutically guided dose escalation (PGDE) method may be applied to determine an appropriate dose of pharmaceutical compositions described herein.

In some embodiments, pharmaceutical compositions described herein can be administered to subjects as monotherapy.

In some embodiments, a pharmaceutical composition provided herein may be administered as part of combination therapy.

In some embodiments, subjects receiving a composition provided herein (e.g., a pharmaceutical composition) may be monitored periodically over a dosing regimen to assess efficacy of the administered treatment. For example, in some embodiments, efficacy of an administered treatment may be assessed periodically, e.g., weekly, biweekly, every 4 weeks, every 5 weeks, every 6 weeks, every 7 weeks, every 8 weeks, or longer.

In some embodiments, polyribonucleotides or pharmaceutical compositions of the present disclosure are administered to a subject in need thereof to induce an immune response against an orthopoxvirus. In some embodiments, the orthopoxvirus is a monkeypox virus, a variola virus, a vaccinia virus, or a cowpox virus.

### IX. Methods of Manufacture

Individual polyribonucleotides can be produced by methods known in the art. For example, in some embodiments, polyribonucleotides can be produced by in vitro transcription, for example, using a DNA template. A plasmid DNA used as a template for in vitro transcription to generate a polyribonucleotide described herein is also within the scope of the present disclosure.

A DNA template is used for in vitro RNA synthesis in the presence of an appropriate RNA polymerase (e.g., a recombinant RNA-polymerase such as a T7 RNA-polymerase) with ribonucleotide triphosphates (e.g., ATP, CTP, GTP, UTP). In some embodiments, polyribonucleotides (e.g., ones described herein) can be synthesized in the presence of modified ribonucleotide triphosphates. By way of example only, in some embodiments, pseudouridine (ψ), N1-methyl-pseudouridine (m1ψ), or 5-methyl-uridine (m5U) can be used to replace uridine triphosphate (UTP). In some embodiments, pseudouridine (ψ) can be used to replace uridine triphosphate (UTP). In some embodiments, N1-methyl-pseudouridine (m1ψ) can be used to replace uridine triphosphate (UTP). In some embodiments, 5-methyl-uridine (m5U) can be used to replace uridine triphosphate (UTP).

As will be clear to those skilled in the art, during in vitro transcription, an RNA polymerase (e.g., as described and/or utilized herein) typically traverses at least a portion of a single-stranded DNA template in the 3′→5′ direction to produce a single-stranded complementary RNA in the 5′→3′ direction.

In some embodiments where a polyribonucleotide comprises a polyA tail, one of those skill in the art will appreciate that such a polyA tail may be encoded in a DNA template, e.g., by using an appropriately tailed PCR primer, or it can be added to a polyribonucleotide after in vitro transcription, e.g., by enzymatic treatment (e.g., using a poly(A) polymerase such as an E. coli Poly(A) polymerase). Suitable poly(A) tails are described herein above. In some embodiments, a poly(A) tail comprises a plurality of A residues interrupted by a linker. In some embodiments, a linker comprises the nucleotide sequence GCATATGAC (SEQ ID NO: 156).

In some embodiments, those skilled in the art will appreciate that addition of a 5′ cap to an RNA (e.g., mRNA) can facilitate recognition and attachment of the RNA to a ribosome to initiate translation and enhances translation efficiency. Those skilled in the art will also appreciate that a 5′ cap can also protect an RNA product from 5′ exonuclease mediated degradation and thus increases half-life. Methods for capping are known in the art; one of ordinary skill in the art will appreciate that in some embodiments, capping may be performed after in vitro transcription in the presence of a capping system (e.g., an enzyme-based capping system such as, e.g., capping enzymes of vaccinia virus). In some embodiments, a cap may be introduced during in vitro transcription, along with a plurality of ribonucleotide triphosphates such that a cap is incorporated into a polyribonucleotide during transcription (also known as co-transcriptional capping). In some embodiments, a GTP fed-batch procedure with multiple additions in the course of the reaction may be used to maintain a low concentration of GTP in order to effectively cap the RNA. Suitable 5′ cap are described herein above. For example, in some embodiments, a 5′ cap comprises m7(3′OMeG)(5′)ppp(5′)(2′OMeA)pG.

Following RNA transcription, a DNA template is digested. In some embodiments, digestion can be achieved with the use of DNase I under appropriate conditions.

In some embodiments, in-vitro transcribed polyribonucleotides may be provided in a buffered solution, for example, in a buffer such as HEPES, a phosphate buffer solution, a citrate buffer solution, an acetate buffer solution; in some embodiments, such solution may be buffered to a pH within a range of, for example, about 6.5 to about 7.5; in some embodiments approximately 7.0. In some embodiments, production of polyribonucleotides may further include one or more of the following steps: purification, mixing, filtration, and/or filling.

In some embodiments, polyribonucleotides can be purified (e.g., in some embodiments after in vitro transcription reaction), for example, to remove components utilized or formed in the course of the production, like, e.g., proteins, DNA fragments, and/or or nucleotides. Various nucleic acid purifications that are known in the art can be used in accordance with the present disclosure. Certain purification steps may be or include, for example, one or more of precipitation, column chromatography (including, e.g., but not limited to anionic, cationic, hydrophobic interaction chromatography (HIC)), solid substrate-based purification (e.g., magnetic bead-based purification). In some embodiments, polyribonucleotides may be purified using magnetic bead-based purification, which in some embodiments may be or comprise magnetic bead-based chromatography. In some embodiments, polyribonucleotides may be purified using hydrophobic interaction chromatography (HIC) and/or diafiltration. In some embodiments, polyribonucleotides may be purified using HIC followed by diafiltration.

In some embodiments, dsRNA may be obtained as side product during in vitro transcription. In some such embodiments, a second purification step may be performed to remove dsRNA contamination. For example, in some embodiments, cellulose materials (e.g., microcrystalline cellulose) may be used to remove dsRNA contamination, for examples in some embodiments in a chromatographic format. In some embodiments, cellulose materials (e.g., microcrystalline cellulose) can be pretreated to inactivate potential RNase contamination, for example in some embodiments by autoclaving followed by incubation with aqueous basic solution, e.g., NaOH. In some embodiments, cellulose materials may be used to purify polyribonucleotides according to methods described in WO 2017/182524, the entire content of which is incorporated herein by reference.

In some embodiments, a batch of polyribonucleotides may be further processed by one or more steps of filtration and/or concentration. For example, in some embodiments, polyribonucleotide(s), for example, after removal of dsRNA contamination, may be further subject to diafiltration (e.g., in some embodiments by tangential flow filtration), for example, to adjust the concentration of polyribonucleotides to a desirable RNA concentration and/or to exchange buffer to a drug substance buffer.

In some embodiments, polyribonucleotides may be processed through 0.2 m filtration before they are filled into appropriate containers.

In some embodiments, polyribonucleotides and compositions thereof may be manufactured in accordance with a process as described herein, or as otherwise known in the art.

In some embodiments, polyribonucleotides and compositions thereof may be manufactured at a large scale. For example, in some embodiments, a batch of polyribonucleotides can be manufactured at a scale of greater than 1 g, greater than 2 g, greater than 3 g, greater than 4 g, greater than 5 g, greater than 6 g, greater than 7 g, greater than 8 g, greater than 9 g, greater than 10 g, greater than 15 g, greater than 20 g, or higher.

In some embodiments, RNA quality control may be performed and/or monitored at any time during production process of polyribonucleotides and/or compositions comprising the same. For example, in some embodiments, RNA quality control parameters, including one or more of RNA identity (e.g., sequence, length, and/or RNA natures), RNA integrity, RNA concentration, residual DNA template, and residual dsRNA, may be assessed and/or monitored after each or certain steps of a polyribonucleotide manufacturing process, e.g., after in vitro transcription, and/or each purification step.

In some embodiments, the stability of polyribonucleotides (e.g., produced by in vitro transcription) and/or compositions comprising two or more RNAs can be assessed under various test storage conditions, for example, at room temperatures vs. fridge or sub-zero temperatures over a period of time (e.g., at least 3 months, at least 6 months, at least 9 months, at least 12 months, or longer). In some embodiments, polyribonucleotides (e.g., ones described herein) and/or compositions thereof may be stored stable at a fridge temperature (e.g., about 4° C. to about 10° C.) for at least 1 month or longer including, at least 2 months, at least 3 months, at least 4 months, at least 5 months, at least 6 months, at least 7 months, at least 8 months, at least 9 months, at least 10 months, at least 11 months, or at least 12 months or longer. In some embodiments, polyribonucleotides (e.g., ones described herein) and/or compositions thereof may be stored stable at a sub-zero temperature (e.g., −20° C. or below) for at least 1 month or longer including, at least 2 months, at least 3 months, at least 4 months, at least 5 months, at least 6 months, at least 7 months, at least 8 months, at least 9 months, at least 10 months, at least 11 months, or at least 12 months or longer. In some embodiments, polyribonucleotides (e.g., ones described herein) and/or compositions thereof may be stored stable at room temperature (e.g., at about 25° C.) for at least 1 month or longer.

In some embodiments, one or more assessments may be utilized during manufacture, or other preparation or use of polyribonucleotides (e.g., as a release test).

In some embodiments, one or more quality control parameters may be assessed to determine whether polyribonucleotides described herein meet or exceed acceptance criteria (e.g., for subsequent formulation and/or release for distribution). In some embodiments, such quality control parameters may include, but are not limited to RNA integrity, RNA concentration, residual DNA template and/or residual dsRNA. Certain methods for assessing RNA quality are known in the art; for example, one of skill in the art will recognize that in some embodiments, one or more analytical tests can be used for RNA quality assessment. Examples of such certain analytical tests may include but are not limited to gel electrophoresis, UV absorption, and/or PCR assay.

In some embodiments, a batch of polyribonucleotides may be assessed for one or more features as described herein to determine next action step(s). For example, a batch of polyribonucleotides can be designated for one or more further steps of manufacturing and/or formulation and/or distribution if RNA quality assessment indicates that such a batch of polyribonucleotides meet or exceed the relevant acceptance criteria. Otherwise, an alternative action can be taken (e.g., discarding the batch) if such a batch of polyribonucleotides does not meet or exceed the acceptance criteria.

In some embodiments, a batch of polyribonucleotides that satisfy assessment results can be utilized for one or more further steps of manufacturing and/or formulation and/or distribution.

In some embodiments, manufacture of a vaccine composition of the present disclosure includes characterizing the efficacy of the vaccine composition by: (i) administering the vaccine composition to a CAST/Ei mouse in one or more doses; (ii) infecting the mouse with an orthopoxvirus (e.g., MPXV); and (iii) measuring a viral titer of the orthopoxvirus (e.g., MPXV) in a sample collected from the mouse. In some embodiments, the vaccine composition is characterized as efficacious if the viral titer is significantly lower than a viral titer of the orthopoxvirus in a control sample.

In some embodiments, manufacture of a vaccine composition of the present disclosure includes testing the ability of the vaccine composition to significantly lower an orthopoxvirus (e.g. MPXV) viral titer measured in a sample collected from a CAST/Ei mouse as compared to a control sample. In some embodiments, the sample is collected from a CAST/Ei mouse that was administered with the vaccine composition, in one or more doses prior to infection with the orthopoxvirus.

In some embodiments, a method of manufacture includes administering a CAST/Ei mouse with one or more doses of the vaccine composition prior to infections with the orthopoxvirus (e.g. MPXV). For example, in some embodiments, a method includes administering one, two, three, four, five, or more doses to the CAST/Ei mouse.

In some embodiments, a dose of the vaccine composition is administered to the CAST/Ei mouse 25 to 75 (e.g., 25 to 75, 30 to 75, 35 to 75, 40 to 75, 45 to 75, 50 to 75, 55 to 75, 60 to 75, 65 to 75, 70 to 75, 25 to 70, 30 to 70, 35 to 70, 40 to 70, 45 to 70, 50 to 70, 55 to 70, 60 to 70, 65 to 70, 25 to 65, 30 to 65, 35 to 65, 40 to 65, 45 to 65, 50 to 65, 55 to 65, 60 to 65, 25 to 60, 30 to 60, 35 to 60, 40 to 60, 45 to 60, 50 to 60, 55 to 60, 25 to 55, 30 to 55, 35 to 55, 40 to 55, 45 to 55, 50 to 55, 25 to 50, 30 to 50, 35 to 50, 40 to 50, 45 to 50, 25 to 45, 30 to 45, 35 to 45, 40 to 45, 25 to 40, 30 to 40, 35 to 40, 25 to 35, 30 to 35, 25 to 30, 50, 51, 52, 53, 54, 55, 56, 57, 58, 59, or 60) days prior to infection with the orthopoxvirus (e.g., MPXV). For example, in some embodiments, a dose of the vaccine composition is administered to the CAST/Ei mouse 56 days prior to infection with the orthopoxvirus (e.g., MPXV).

In some embodiments, a method of manufacture includes administering two doses of the vaccine composition to the CAST/Ei mouse. In some embodiments a dose is administered to the CAST/Ei mouse 25 to 75 75 (e.g., 25 to 75, 30 to 75, 35 to 75, 40 to 75, 45 to 75, 50 to 75, 55 to 75, 60 to 75, 65 to 75, 70 to 75, 25 to 70, 30 to 70, 35 to 70, 40 to 70, 45 to 70, 50 to 70, 55 to 70, 60 to 70, 65 to 70, 25 to 65, 30 to 65, 35 to 65, 40 to 65, 45 to 65, 50 to 65, 55 to 65, 60 to 65, 25 to 60, 30 to 60, 35 to 60, 40 to 60, 45 to 60, 50 to 60, 55 to 60, 25 to 55, 30 to 55, 35 to 55, 40 to 55, 45 to 55, 50 to 55, 25 to 50, 30 to 50, 35 to 50, 40 to 50, 45 to 50, 25 to 45, 30 to 45, 35 to 45, 40 to 45, 25 to 40, 30 to 40, 35 to 40, 25 to 35, 30 to 35, 25 to 30, 50, 51, 52, 53, 54, 55, 56, 57, 58, 59, or 60) days prior to infection with the orthopoxvirus (e.g., MPXV), and an additional dose is administered to the CAST/Ei mouse 20 to 50 days (e.g., 20 to 50, 25 to 50, 30 to 50, 35 to 50, 40 to 50, 45 to 50, 20 to 45, 25 to 45, 30 to 45, 35 to 45, 40 to 45, 20 to 40, 25 to 40, 30 to 40, 35 to 40, 20 to 35, 25 to 35, 30 to 35, 20 to 30, 25 to 30, 30, 31, 32, 33, 34, 35, 36, 37, 38, 39, or 40) days prior to infection with the orthopoxvirus. For example, in some embodiments, a method of manufacture includes administering the dose of the vaccine composition and the additional dose of the vaccine composition to the CAST/Ei mouse at 56 days and 35 days prior to orthopox (e.g., MPXV) infection, respectively.

In some embodiments, the one or more doses of the vaccine composition includes 0.5 to 10 μg (e.g., 0.5 to 10, 1 to 10, 1.5 to 10, 2 to 10, 2.5 to 10, 3 to 10, 3.5 to 10, 4 to 10, 4.5 to 10, 5 to 10, 5.5 to 10, 6 to 10, 6.5 to 10, 7 to 10, 7.5 to 10, 8 to 10, 8.5 to 10, 9 to 10, 9.5 to 10, 0.5 to 9, 1 to 9, 1.5 to 9, 2 to 9, 2.5 to 9, 3 to 9, 3.5 to 9, 4 to 9, 4.5 to 9, 5 to 9, 5.5 to 9, 6 to 9, 6.5 to 9, 7 to 9, 7.5 to 9, 8 to 9, 8.5 to 9, 0.5 to 8, 1 to 8, 1.5 to 8, 2 to 8, 2.5 to 8, 3 to 8, 3.5 to 8, 4 to 8, 4.5 to 8, 5 to 8, 5.5 to 8, 6 to 8, 6.5 to 8, 7 to 8, 7.5 to 8, 0.5 to 7, 1 to 7, 1.5 to 7, 2 to 7, 2.5 to 7, 3 to 7, 3.5 to 7, 4 to 7, 4.5 to 7, 5 to 7, 5.5 to 7, 6 to 7, 6.5 to 7, 0.5 to 6, 1 to 6, 1.5 to 6, 2 to 6, 2.5 to 6, 3 to 6, 3.5 to 6, 4 to 6, 4.5 to 6, 5 to 6, 5.5 to 6, 0.5 to 5, 1 to 5, 1.5 to 5, 2 to 5, 2.5 to 5, 3 to 5, 3.5 to 5, 4 to 5, 4.5 to 5, 0.5 to 4, 1 to 4, 1.5 to 4, 2 to 4, 2.5 to 4, 3 to 4, 3.5 to 4, 0.5 to 3, 1 to 3, 1.5 to 3, 2 to 3, 2.5 to 3, 0.5 to 2, 1 to 2, 1.5 to 2, 0.5, 1, 2, 3, 4, 5, 6, 7, 8, 9, or 10 μg) of a monkeypox antigen-encoding polyribonucleotide of the present disclosure. In some embodiments, the one or more doses of the vaccine composition includes 1 μg of the polyribonucleotide. In some embodiments, the one or more doses of the vaccine composition includes 4 μg of the polyribonucleotide. In some embodiments, a method includes administering two doses of the vaccine composition and each of the doses includes 4 μg of the polyribonucleotide.

In some embodiments, a method of manufacture includes administering one or more doses of the vaccine composition to the CAST/Ei mouse intramuscularly, intravenously, or intranasally. For example, in some embodiments, the method of manufacture includes administering one or more doses of the vaccine composition to the CAST/Ei mouse intramuscularly.

In some embodiments, a method of manufacture includes infecting the CAST/Ei mouse with 6×10⁶ PFU to 1.2×10⁷ PFU (e.g., 6×10⁶ PFU to 1.2×10⁷ PFU, 7×10⁶ PFU to 1.2×10⁷ PFU, 8×10⁶ PFU to 1.2×10⁷ PFU, 9×10⁶ PFU to 1.2×10⁷ PFU, 1×10⁷ PFU to 1.2×10⁷ PFU, 1.1×10⁷ PFU to 1.2×10⁷ PFU, 6×10⁶ PFU to 1.1×10⁷ PFU, 7×10⁶ PFU to 1.1×10⁷ PFU, 8×10⁶ PFU to 1.1×10⁷ PFU, 9×10⁶ PFU to 1.1×10⁷ PFU, 1×10⁷ PFU to 1.1×10⁷ PFU, 6×10⁶ PFU to 1×10⁷ PFU, 7×10⁶ PFU to 1×10⁷ PFU, 8×10⁶ PFU to 1×10⁷ PFU, 9×10⁶ PFU to 1×10⁷ PFU, 6×10⁶ PFU to 9×10⁶ PFU, 7×10⁶ PFU to 9×10⁶ PFU, 8×10⁶ PFU to 9×10⁶ PFU, 6×10⁶ PFU to 8×10⁶ PFU, 7×10⁶ PFU to 8×10⁶ PFU, 6×10⁶ PFU to 7×10⁶ PFU, 6×10⁶ PFU, 7×10⁶ PFU, 8×10⁶ PFU, 9×10⁶ PFU, 1×10⁷ PFU, 1.1×10⁷ PFU, or 1.2×10⁷ PFU) of the orthopoxvirus. For example, in some embodiments, the method of manufacture includes infecting the CAST/Ei mouse with 9×10⁶ PFU of orthopoxvirus (e.g. MPXV).

In some embodiments, a method of manufacture includes infecting the CAST/Ei mouse with orthopoxvirus (e.g. MPXV) intranasally.

In some embodiments, a method of manufacture includes collecting one or more (e.g., one, two, three, four, five, six, seven, eight, nine, ten, or more) samples from the CAST/Ei mouse. In some embodiments, one or more samples from the CAST/Ei mouse is/are collected 1 to 10 days (e.g., 1 to 10, 2 to 10, 3 to 10, 4 to 10, 5 to 10, 6 to 10, 7 to 10, 8 to 10, 9 to 10, 1 to 9, 2 to 9, 3 to 9, 4 to 9, 5 to 9, 6 to 9, 7 to 9, 8 to 9, 1 to 8, 2 to 8, 3 to 8, 4 to 8, 5 to 8, 6 to 8, 7 to 8, 1 to 7, 2 to 7, 3 to 7, 4 to 7, 5 to 7, 6 to 7, 1 to 6, 2 to 6, 3 to 6, 4 to 6, 5 to 6, 1 to 5, 2 to 5, 3 to 5, 4 to 5, 1 to 4, 2 to 4, 3 to 4, 1 to 3, 2 to 3, 1 to 2, 1, 2, 3, 4, 5, 6, 7, 8, 9, or 10 days post infection with the orthopoxvirus (e.g. MPXV). For example in some embodiments, two samples are collected from the CAST/Ei mouse at 3 days and 7 days post infection with orthopoxvirus (e.g. MPXV).In some embodiments, a method of manufacture includes collecting one or more (e.g., one, two, three, four, five, six, seven, eight, nine, ten, or more) samples from the CAST/Ei mouse post infection with orthopoxvirus, where the sample is a lung tissue sample, a bronchiolar lavage sample, a blood sample, a urine sample, or a fecal sample. For example, in some embodiments, the one or more samples is a homogenized lung tissue sample.

In some embodiments, a method of manufacture includes comparing one or more sample collected from the orthopox-infected CAST/Ei mouse with a control sample. As understood by a person of skill in the art, a control sample can include a sample collected from an orthopox-infected CAST/Ei mouse treated under the same conditions as a mouse administered with a vaccine composition, but without having been administered with the vaccine composition. For example, in some embodiments, a method includes comparing the viral titer of orthopox in a lung tissue sample collected from an orthopox-infected CAST/Ei mouse administered with one or more doses of vaccine composition, with the viral titer of orthopox in a lung tissue sample collected from an orthopox-infected CAST/Ei mouse that has not been administered with one or more doses of vaccine composition.

In some embodiments, a method of manufacture includes infecting a CAST/Ei mouse with a monkeypox virus, vaccinia virus, variola virus, or cowpox virus. For example, in some embodiments, a method of manufacture includes infecting a CAST/Ei mouse with a monkeypox virus.

In some embodiments, a method of manufacture includes characterizing whether a vaccine composition significantly lowers the viral titer of orthopox (e.g., MPXV) in a sample collected from an orthopox-infected CAST/Ei mouse as compared to a control sample. In some embodiments, a vaccine composition significantly lowers the viral titer of orthopox (e.g., MPXV) in a sample, when the viral titer is at least 40%, at least 45%, at least 50%, at least 55%, at least 60%, at least 65%, at least 70%, at least 75%, at least 80%, at least 85%, at least 90%, or at least 95% lower than the viral titer of orthopoxvirus (e.g., MPXV) in a control sample.

### X. DNA Constructs

Among other things, the present disclosure provides DNA constructs, for example that may encode one or more antigens or fragments thereof as described herein, or components thereof. In some embodiments, DNA constructs provided by and/or utilized in accordance with the present disclosure are comprised in a vector.

Non-limiting examples of a vector include plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). In some embodiments, a vector is an expression vector. In some embodiments, a vector is a cloning vector. In general, a vector is a nucleic acid construct that can receive or otherwise become linked to a nucleic acid element of interest (e.g., a construct that is or encodes a payload, or that imparts a particular functionality, etc.)

Expression vectors, which may be plasmid or viral or other vectors, typically include an expressible sequence of interest (e.g., a coding sequence) that is functionally linked with one or more control elements (e.g., promoters, enhancers, transcription terminators, etc). Typically, such control elements are selected for expression in a system of interest. In some embodiments, a system is ex vivo (e.g., an in vitro transcription system); in some embodiments, a system is in vivo (e.g., a bacterial, yeast, plant, insect, fish, vertebrate, mammalian cell or tissue, etc).

Cloning vectors are generally used to modify, engineer, and/or duplicate (e.g., by replication in vivo, for example in a simple system such as bacteria or yeast, or in vitro, such as by amplification such as polymerase chain reaction or other amplification process). In some embodiments, a cloning vector may lack expression signals.

In many embodiments, a vector may include replication elements such as primer binding site(s) and/or origin(s) of replication. In many embodiments, a vector may include insertion or modification sites such as restriction endonuclease recognition sites and/or guide RNA binding sites, etc.

In some embodiments, a vector is a viral vector (e.g., an AAV vector). In some embodiments, a vector is a non-viral vector. In some embodiments, a vector is a plasmid.

Those skilled in the art are aware of a variety of technologies useful for the production of recombinant polynucleotides (e.g., DNA or RNA) as described herein. For example, restriction digestion, reverse transcription, amplification (e.g., by polymerase chain reaction), Gibson assembly, etc., are well established and useful tools and technologies. Alternatively or additionally, certain nucleic acids may be prepared or assembled by chemical and/or enzymatic synthesis. In some embodiments, a combination of known methods is utilized to prepare a recombinant polynucleotide.

In some embodiments, polynucleotide(s) of the present disclosure are included in a DNA construct (e.g., a vector) amenable to transcription and/or translation.

In some embodiments, an expression vector comprises a polynucleotide that encodes proteins and/or polypeptides of the present disclosure operatively linked to a sequence or sequences that control expression (e.g., promoters, start signals, stop signals, polyadenylation signals, activators, repressors, etc.). In some embodiments, a sequence or sequences that control expression are selected to achieve a desired level of expression. In some embodiments, more than one sequence that controls expression (e.g., promoters) are utilized. In some embodiments, more than one sequence that controls expression (e.g., promoters) are utilized to achieve a desired level of expression of a plurality of polynucleotides that encode a plurality proteins and/or polypeptides. In some embodiments, a plurality of recombinant proteins and/or polypeptides are expressed from the same vector (e.g., a bi-cistronic vector, a tri-cistronic vector, multi-cistronic). In some embodiments, a plurality of polypeptides are expressed, each of which is expressed from a separate vector.

In some embodiments, an expression vector comprising a polynucleotide of the present disclosure is used to produce a RNA and/or protein and/or polypeptide in a host cell. In some embodiments, a host cell may be in vitro (e.g., a cell line)—for example a cell or cell line (e.g., Human Embryonic Kidney (HEK cells), Chinese Hamster Ovary cells, etc.) suitable for producing polynucleotides of the present disclosure and proteins and/or polypeptides encoded by said polynucleotides.

In some embodiments, an expression vector is an RNA expression vector. In some embodiments, an RNA expression vector comprises a polynucleotide template used to produce a RNA in cell-free enzymatic mix. In some embodiments, an RNA expression vector comprising a polynucleotide template is enzymatically linearized prior to in vitro transcription. In some embodiments, a polynucleotide template is generated through PCR as a linear polynucleotide template. In some embodiments, a linearized polynucleotide is mixed with enzymes suitable for RNA synthesis, RNA capping and/or purification. In some embodiments, the resulting RNA is suitable for producing proteins encoded by the RNA.

A variety of methods are known in the art to introduce an expression vector into host cells. In some embodiments, a vector may be introduced into host cells using transfection. In some embodiments, transfection is completed, for example, using calcium phosphate transfection, lipofection, or polyethylenimine-mediated transfection. In some embodiments, a vector may be introduced into a host cell using transduction.

In some embodiments, transformed host cells are cultured following introduction of a vector into a host cell to allow for expression of said recombinant polynucleotides. In some embodiments, a transformed host cells are cultured for at least 12 hours, 16 hours, 20 hours, 24 hours, 28 hours, 32 hours, 36 hours 40 hours, 44 hours, 48 hours, 52 hours, 56 hours, 60 hours, 64 hours, 68 hours, 72 hours or longer. Transformed host cells are cultured in growth conditions (e.g., temperature, carbon-dioxide levels, growth medium) in accordance with the requirements of a host cell selected. A skilled artisan would recognize culture conditions for host cells selected are well known in the art.

### Exemplary Embodiments

Embodiment 1. A polyribonucleotide encoding one or more monkeypox antigens or fragments thereof.

Embodiment 2. A polyribonucleotide encoding one or more monkeypox antigens or fragments thereof, wherein the one or more monkeypox antigens or fragments thereof comprise:
(i) one or more extracellular-enveloped virus (EEV)-specific antigens;
(ii) one or more intracellular mature virions (IMV)-specific antigens; or
(iii) a combination thereof.

Embodiment 3. A polyribonucleotide encoding one or more monkeypox antigens or fragments thereof, wherein
(i) the EEV-specific antigens comprise M1R, E8L, H3L, or a fragment thereof, and/or
(ii) the IMV-specific antigens comprise A35R, B6R, or a fragment thereof.

Embodiment 4. A polyribonucleotide encoding one or more monkeypox antigens or fragments thereof, wherein the one or more monkeypox antigens or fragments thereof comprise one or more of:
(i) A35R or a fragment of A35R,
(ii) B6R or a fragment of B6R,
(iii) M1R or a fragment of M1R,
(iv) H3L or a fragment of H3L, and
(v) E8L or a fragment of E8L.

Embodiment 5. A polyribonucleotide encoding one or more monkeypox antigens or fragments thereof, wherein the one or more monkeypox antigens or fragments thereof comprise two to five of:
(i) A35R or a fragment of A35R,
(ii) B6R or a fragment of B6R,
(iii) M1R or a fragment of M1R,
(iv) H3L or a fragment of H3L, and
(v) E8L or a fragment of E8L.

Embodiment 6. A polyribonucleotide encoding one or more monkeypox antigens or fragments thereof, wherein the one or more monkeypox antigens or fragments thereof comprise:
(i) B6R or a fragment of B6R, and
(ii) M1R or a fragment of M1R.

Embodiment 7. A polyribonucleotide encoding one or more monkeypox antigens or fragments thereof, wherein the one or more monkeypox antigens or fragments thereof comprise:
(i) A35R or a fragment of A35R,
(ii) B6R or a fragment of B6R, and
(iii) M1R or a fragment of M1R.

Embodiment 8. A polyribonucleotide encoding one or more monkeypox antigens or fragments thereof, wherein the one or more monkeypox antigens or fragments thereof comprise:
(i) A35R or a fragment of A35R,
(ii) B6R or a fragment of B6R,
(iii) M1R or a fragment of M1R, and
(iv) E8L or a fragment of E8L.

Embodiment 9. A polyribonucleotide encoding one or more monkeypox antigens or fragments thereof, wherein the one or more monkeypox antigens or fragments thereof comprise:
(i) A35R or a fragment of A35R,
(ii) B6R or a fragment of B6R,
(iii) M1R or a fragment of M1R, and
(iv) H3L or a fragment of H3L.

Embodiment 10. A polyribonucleotide encoding one or more monkeypox antigens or fragments thereof, wherein the one or more monkeypox antigens or fragments thereof comprise one or more of:
(i) A35R or a fragment of A35R,
(ii) B6R or a fragment of B6R,
(iii) M1R or a fragment of M1R,
(iv) E8L or a fragment of E8L,
(v) H3L or a fragment of H3L,
(vi) A28L or a fragment of A28L, and
(vii) A29L or a fragment of A29L.

Embodiment 11. A polyribonucleotide encoding one or more monkeypox antigens or fragments thereof, wherein the one or more monkeypox antigens or fragments thereof comprise two to five of:
(i) A35R or a fragment of A35R,
(ii) B6R or a fragment of B6R,
(iii) M1R or a fragment of M1R,
(iv) E8L or a fragment of E8L,
(v) H3L or a fragment of H3L,
(vi) A28L or a fragment of A28L, and
(vii) A29L or a fragment of A29L.

Embodiment 12. A polyribonucleotide encoding one or more monkeypox antigens or fragments thereof, wherein the one or more monkeypox antigens or fragments thereof comprise one of:
(i) A45L or a fragment of A45L,
(ii) B9R or a fragment of B9R,
(iii) B16R or a fragment of B16R,
(iv) C10L or a fragment of C10L,
(v) C21L or a fragment of C21L,
(vi) E7R or a fragment of E7R,
(vii) F3L or a fragment of F3L,
(viii) F4L or a fragment of F4L,
(ix) G6R or a fragment of G6R,
(x) H5R or a fragment of H5R,
(xi) I3L or a fragment of I3L,
(xii) O2L or a fragment of O2L,
(xiii) Q1L or a fragment of Q1L,
(xiv) B12R or a fragment of B12R, and
(xv) C17L or a fragment of C17L.

Embodiment 13 A polyribonucleotide encoding one or more monkeypox antigens or fragments thereof, wherein the one or more monkeypox antigens or fragments thereof comprise two of:
(i) A45L or a fragment of A45L,
(ii) B9R or a fragment of B9R,
(iii) B16R or a fragment of B16R,
(iv) C10L or a fragment of C10L,
(v) C21L or a fragment of C21L,
(vi) E7R or a fragment of E7R,
(vii) F3L or a fragment of F3L,
(viii) F4L or a fragment of F4L,
(ix) G6R or a fragment of G6R,
(x) H5R or a fragment of H5R,
(xi) I3L or a fragment of I3L,
(xii) O2L or a fragment of O2L,
(xiii) Q1L or a fragment of Q1L,
(xiv) B12R or a fragment of B12R, and
(xv) C17L or a fragment of C17L.

Embodiment 14. A polyribonucleotide encoding one or more monkeypox antigens or fragments thereof, wherein the one or more monkeypox antigens or fragments thereof comprise three of:
(i) A45L or a fragment of A45L,
(ii) B9R or a fragment of B9R,
(iii) B16R or a fragment of B16R,
(iv) C10L or a fragment of C10L,
(v) C21L or a fragment of C21L,
(vi) E7R or a fragment of E7R,
(vii) F3L or a fragment of F3L,
(viii) F4L or a fragment of F4L,
(ix) G6R or a fragment of G6R,
(x) H5R or a fragment of H5R,
(xi) I3L or a fragment of I3L,
(xii) O2L or a fragment of O2L,
(xiii) Q1L or a fragment of Q1L,
(xiv) B12R or a fragment of B12R, and
(xv) C17L or a fragment of C17L.

Embodiment 15. A polyribonucleotide encoding one or more monkeypox antigens or fragments thereof, wherein the one or more monkeypox antigens or fragments thereof comprise four of.
(i) A45L or a fragment of A45L,
(ii) B9R or a fragment of B9R,
(iii) B16R or a fragment of B16R,
(iv) C10L or a fragment of C10L,
(v) C21L or a fragment of C21L,
(vi) E7R or a fragment of E7R,
(vii) F3L or a fragment of F3L,
(viii) F4L or a fragment of F4L,
(ix) G6R or a fragment of G6R,
(x) H5R or a fragment of H5R,
(xi) I3L or a fragment of I3L,
(xii) O2L or a fragment of O2L,
(xiii) Q1L or a fragment of Q1L,
(xiv) B12R or a fragment of B12R, and
(xv) C17L or a fragment of C17L.

Embodiment 16. A polyribonucleotide encoding one or more monkeypox antigens or fragments thereof, wherein the one or more monkeypox antigens or fragments thereof comprise five of.
(i) A45L or a fragment of A45L,
(ii) B9R or a fragment of B9R,
(iii) B16R or a fragment of B16R,
(iv) C10L or a fragment of C10L,
(v) C21L or a fragment of C21L,
(vi) E7R or a fragment of E7R,
(vii) F3L or a fragment of F3L,
(viii) F4L or a fragment of F4L,
(ix) G6R or a fragment of G6R,
(x) H5R or a fragment of H5R,
(xi) I3L or a fragment of I3L,
(xii) O2L or a fragment of O2L,
(xiii) Q1L or a fragment of Q1L,
(xiv) B12R or a fragment of B12R, and
(xv) C17L or a fragment of C17L.

Embodiment 17. A polyribonucleotide encoding one or more monkeypox antigens or fragments thereof, wherein the one or more monkeypox antigens or fragments thereof comprise six of:
(i) A45L or a fragment of A45L,
(ii) B9R or a fragment of B9R,
(iii) B16R or a fragment of B16R,
(iv) C10L or a fragment of C10L,
(v) C21L or a fragment of C21L,
(vi) E7R or a fragment of E7R,
(vii) F3L or a fragment of F3L,
(viii) F4L or a fragment of F4L,
(ix) G6R or a fragment of G6R,
(x) H5R or a fragment of H5R,
(xi) I3L or a fragment of I3L,
(xii) O2L or a fragment of O2L,
(xiii) Q1L or a fragment of Q1L,
(xiv) B12R or a fragment of B12R, and
(xv) C17L or a fragment of C17L.

Embodiment 18. A polyribonucleotide encoding one or more monkeypox antigens or fragments thereof, wherein the one or more monkeypox antigens or fragments thereof comprise seven of:
(i) A45L or a fragment of A45L,
(ii) B9R or a fragment of B9R,
(iii) B16R or a fragment of B16R,
(iv) C10L or a fragment of C10L,
(v) C21L or a fragment of C21L,
(vi) E7R or a fragment of E7R,
(vii) F3L or a fragment of F3L,
(viii) F4L or a fragment of F4L,
(ix) G6R or a fragment of G6R,
(x) H5R or a fragment of H5R,
(xi) I3L or a fragment of I3L,
(xii) O2L or a fragment of O2L,
(xiii) Q1L or a fragment of Q1L,
(xiv) B12R or a fragment of B12R, and
(xv) C17L or a fragment of C17L.

Embodiment 19. A polyribonucleotide encoding one or more monkeypox antigens or fragments thereof, wherein the one or more monkeypox antigens or fragments thereof comprise eight of:
(i) A45L or a fragment of A45L,
(ii) B9R or a fragment of B9R,
(iii) B16R or a fragment of B16R,
(iv) C10L or a fragment of C10L,
(v) C21L or a fragment of C21L,
(vi) E7R or a fragment of E7R,
(vii) F3L or a fragment of F3L,
(viii) F4L or a fragment of F4L,
(ix) G6R or a fragment of G6R,
(x) H5R or a fragment of H5R,
(xi) I3L or a fragment of I3L,
(xii) O2L or a fragment of O2L,
(xiii) Q1L or a fragment of Q1L,
(xiv) B12R or a fragment of B12R, and
(xv) C17L or a fragment of C17L.

Embodiment 20. A polyribonucleotide encoding one or more monkeypox antigens or fragments thereof, wherein the one or more monkeypox antigens or fragments thereof comprise nine of.
(i) A45L or a fragment of A45L,
(ii) B9R or a fragment of B9R,
(iii) B16R or a fragment of B16R,
(iv) C10L or a fragment of C10L,
(v) C21L or a fragment of C21L,
(vi) E7R or a fragment of E7R,
(vii) F3L or a fragment of F3L,
(viii) F4L or a fragment of F4L,
(ix) G6R or a fragment of G6R,
(x) H5R or a fragment of H5R,
(xi) I3L or a fragment of I3L,
(xii) O2L or a fragment of O2L,
(xiii) Q1L or a fragment of Q1L,
(xiv) B12R or a fragment of B12R, and
(xv) C17L or a fragment of C17L.

Embodiment 21. A polyribonucleotide encoding one or more monkeypox antigens or fragments thereof, wherein the one or more monkeypox antigens or fragments thereof comprise ten of.
(i) A45L or a fragment of A45L,
(ii) B9R or a fragment of B9R,
(iii) B16R or a fragment of B16R,
(iv) C10L or a fragment of C10L,
(v) C21L or a fragment of C21L,
(vi) E7R or a fragment of E7R,
(vii) F3L or a fragment of F3L,
(viii) F4L or a fragment of F4L,
(ix) G6R or a fragment of G6R,
(x) H5R or a fragment of H5R,
(xi) I3L or a fragment of I3L,
(xii) O2L or a fragment of O2L,
(xiii) Q1L or a fragment of Q1L,
(xiv) B12R or a fragment of B12R, and
(xv) C17L or a fragment of C17L.

Embodiment 22. A polyribonucleotide encoding one or more monkeypox antigens or fragments thereof, wherein the one or more monkeypox antigens or fragments thereof comprise eleven of:
(i) A45L or a fragment of A45L,
(ii) B9R or a fragment of B9R,
(iii) B16R or a fragment of B16R,
(iv) C10L or a fragment of C10L,
(v) C21L or a fragment of C21L,
(vi) E7R or a fragment of E7R,
(vii) F3L or a fragment of F3L,
(viii) F4L or a fragment of F4L,
(ix) G6R or a fragment of G6R,
(x) H5R or a fragment of H5R,
(xi) I3L or a fragment of I3L,
(xii) O2L or a fragment of O2L,
(xiii) Q1L or a fragment of Q1L,
(xiv) B12R or a fragment of B12R, and
(xv) C17L or a fragment of C17L.

Embodiment 23. A polyribonucleotide encoding one or more monkeypox antigens or fragments thereof, wherein the one or more monkeypox antigens or fragments thereof comprise twelve, thirteen, fourteen, or fifteen of:
(i) A45L or a fragment of A45L,
(ii) B9R or a fragment of B9R,
(iii) B16R or a fragment of B16R,
(iv) C10L or a fragment of C10L,
(v) C21L or a fragment of C21L,
(vi) E7R or a fragment of E7R,
(vii) F3L or a fragment of F3L,
(viii) F4L or a fragment of F4L,
(ix) G6R or a fragment of G6R,
(x) H5R or a fragment of H5R,
(xi) I3L or a fragment of I3L,
(xii) O2L or a fragment of O2L,
(xiii) Q1L or a fragment of Q1L,
(xiv) B12R or a fragment of B12R, and
(xv) C17L or a fragment of C17L.

Embodiment 24. A polyribonucleotide encoding one or more monkeypox antigens or fragments thereof, wherein the one or more monkeypox antigens or fragments thereof comprise two or more of:
(i) A29L or a fragment of A29L,
(ii) A35R or a fragment of A35R,
(iii) B6R or a fragment of B6R,
(iv) M1R or a fragment of M1R,
(v) E8L or a fragment of E8L,
(vi) H3L or a fragment of H3L.
(vii) A45L or a fragment of A45L,
(viii) B9R or a fragment of B9R,
(ix) B16R or a fragment of B16R,
(x) C10L or a fragment of C10L,
(xi) C21L or a fragment of C21L,
(xii) E7R or a fragment of E7R,
(xiii) F3L or a fragment of F3L,
(xiv) F4L or a fragment of F4L,
(xv) G6R or a fragment of G6R,
(xvi) H5R or a fragment of H5R,
(xvii) I3L or a fragment of I3L,
(xviii) O2L or a fragment of O2L,
(xix) Q1L or a fragment of Q1L,
(xx) B12R or a fragment of B12R,
(xxi) A28L or a fragment of A28L, and
(xxii) C17L or a fragment of C17L.

Embodiment 25. A polyribonucleotide encoding one or more monkeypox antigens or fragments thereof, wherein the one or more monkeypox antigens or fragments thereof comprises a monkeypox A29L or a fragment thereof.

Embodiment 26. A polyribonucleotide encoding one or more monkeypox antigens or fragments thereof, wherein the monkeypox A29L or fragment thereof comprises an amino acid sequence that is at least 80%, at least 85%, at least 90%, or at least 95% identical to an amino acid sequence according to one of SEQ ID NOs: 1-10 or a portion thereof.

Embodiment 27. A polyribonucleotide encoding one or more monkeypox antigens or fragments thereof, wherein the monkeypox A29L or fragment thereof comprises or consists of an amino acid sequence that is identical to an amino acid sequence according to one of SEQ ID NOs: 1-10 or a corresponding portion thereof.

Embodiment 28. A polyribonucleotide encoding one or more monkeypox antigens or fragments thereof, wherein the one or more monkeypox antigens or fragments thereof comprises a monkeypox A35R or a fragment thereof.

Embodiment 29. A polyribonucleotide encoding one or more monkeypox antigens or fragments thereof, wherein the monkeypox A35R or fragment thereof comprises an amino acid sequence that is at least 80%, at least 85%, at least 90%, or at least 95% identical to an amino acid sequence according to one of SEQ ID NOs: 11-20 or a portion thereof.

Embodiment 30 A polyribonucleotide encoding one or more monkeypox antigens or fragments thereof, wherein the monkeypox A35R or fragment thereof comprises or consists of an amino acid sequence that is identical to an amino acid sequence according to one of SEQ ID NOs: 11-20 or a corresponding portion thereof.

Embodiment 31. A polyribonucleotide encoding one or more monkeypox antigens or fragments thereof, wherein the one or more monkeypox antigens or fragments thereof comprises a monkeypox B6R or a fragment thereof.

Embodiment 32. A polyribonucleotide encoding one or more monkeypox antigens or fragments thereof, wherein the monkeypox B6R or fragment thereof comprises an amino acid sequence that is at least 80%, at least 85%, at least 90%, or at least 95% identical to an amino acid sequence according to one of SEQ ID NOs: 21-30 or a portion thereof.

Embodiment 33. A polyribonucleotide encoding one or more monkeypox antigens or fragments thereof, wherein the monkeypox B6R or fragment thereof comprises or consists of an amino acid sequence that is identical to an amino acid sequence according to one of SEQ ID NOs: 21-30 or a corresponding portion thereof.

Embodiment 34. A polyribonucleotide encoding one or more monkeypox antigens or fragments thereof, wherein the one or more monkeypox antigens or fragments thereof comprises a monkeypox M1R or a fragment thereof.

Embodiment 35. A polyribonucleotide encoding one or more monkeypox antigens or fragments thereof, wherein the monkeypox M1R or fragment thereof comprises an amino acid sequence that is at least 80%, at least 85%, at least 90%, or at least 95% identical to an amino acid sequence according to one of SEQ ID NOs: 31-40 or a portion thereof.

Embodiment 36. A polyribonucleotide encoding one or more monkeypox antigens or fragments thereof, wherein the monkeypox M1R or fragment thereof comprises or consists of an amino acid sequence that is identical to an amino acid sequence according to one of SEQ ID NOs: 31-40 or a corresponding portion thereof.

Embodiment 37. A polyribonucleotide encoding one or more monkeypox antigens or fragments thereof, wherein the one or more monkeypox antigens or fragments thereof comprises a monkeypox E8L or a fragment thereof.

Embodiment 38. A polyribonucleotide encoding one or more monkeypox antigens or fragments thereof, wherein the monkeypox E8L or fragment thereof comprises an amino acid sequence that is at least 80%, at least 85%, at least 90%, or at least 95% identical to an amino acid sequence according to one of SEQ ID NOs: 41-50 or a portion thereof.

Embodiment 39. A polyribonucleotide encoding one or more monkeypox antigens or fragments thereof, wherein the monkeypox E8L or fragment thereof comprises or consists of an amino acid sequence that is identical to an amino acid sequence according to one of SEQ ID NOs: 41-50 or a corresponding portion thereof.

Embodiment 40. A polyribonucleotide encoding one or more monkeypox antigens or fragments thereof, wherein the one or more monkeypox antigens or fragments thereof comprises a monkeypox H3L or a fragment thereof.

Embodiment 41. A polyribonucleotide encoding one or more monkeypox antigens or fragments thereof, wherein the monkeypox H3L or fragment thereof comprises an amino acid sequence that is at least 80%, at least 85%, at least 90%, or at least 95% identical to an amino acid sequence according to one of SEQ ID NOs: 51-60 or a portion thereof.

Embodiment 42. A polyribonucleotide encoding one or more monkeypox antigens or fragments thereof, wherein the monkeypox H3L or fragment thereof comprises or consists of an amino acid sequence that is identical to an amino acid sequence according to one of SEQ ID NOs: 51-60 or a corresponding portion thereof.

Embodiment 43. A polyribonucleotide encoding one or more monkeypox antigens or fragments thereof, wherein the one or more monkeypox antigens or fragments thereof comprises an M1R amino acid sequence that is at least 80%, at least 85%, at least 90%, or at least 95% identical, or is 100% identical, to an amino acid sequence according to SEQ ID NO: 158 and/or at least 80%, at least 85%, at least 90%, or at least 95% identical, or is 100% identical, to an amino acid sequence according to SEQ ID NO: 258.

Embodiment 44. A polyribonucleotide encoding one or more monkeypox antigens or fragments thereof, wherein the M1R amino acid sequence is operably linked with a signal peptide.

Embodiment 45. A polyribonucleotide encoding one or more monkeypox antigens or fragments thereof, wherein the signal peptide has an amino acid sequence according to SEQ ID NO: 160.

Embodiment 46. A polyribonucleotide encoding one or more monkeypox antigens or fragments thereof, wherein the polyribonucleotide encodes an antigen construct having an amino acid sequence that is at least 80%, at least 85%, at least 90%, or at least 95% identical, or is 100% identical, to an amino acid sequence according to SEQ ID NO: 162.

Embodiment 47. A polyribonucleotide encoding one or more monkeypox antigens or fragments thereof, wherein the one or more monkeypox antigens or fragments thereof comprises an A29L amino acid sequence that is at least 80%, at least 85%, at least 90%, or at least 95% identical, or is 100% identical, to an amino acid sequence according to SEQ ID NO: 164.

Embodiment 48. A polyribonucleotide encoding one or more monkeypox antigens or fragments thereof, wherein the A29L amino acid sequence is operably linked with a signal peptide.

Embodiment 49. A polyribonucleotide encoding one or more monkeypox antigens or fragments thereof, wherein the signal peptide has an amino acid sequence according to SEQ ID NO: 160.

Embodiment 50. A polyribonucleotide encoding one or more monkeypox antigens or fragments thereof, wherein the polyribonucleotide encodes an antigen construct having an amino acid sequence that is at least 80%, at least 85%, at least 90%, or at least 95% identical, or is 100% identical, to an amino acid sequence according to SEQ ID NO: 166.

Embodiment 51. A polyribonucleotide encoding one or more monkeypox antigens or fragments thereof, wherein the one or more monkeypox antigens or fragments thereof comprises an A29L amino acid sequence that is at least 80%, at least 85%, at least 90%, or at least 95% identical, or is 100% identical, to an amino acid sequence according to SEQ ID NO: 168.

Embodiment 52. A polyribonucleotide encoding one or more monkeypox antigens or fragments thereof, wherein the A29L amino acid sequence is operably linked with a signal peptide.

Embodiment 53. A polyribonucleotide encoding one or more monkeypox antigens or fragments thereof, wherein the signal peptide has an amino acid sequence according to SEQ ID NO: 160.

Embodiment 54. A polyribonucleotide encoding one or more monkeypox antigens or fragments thereof, wherein the polyribonucleotide encodes an antigen construct having an amino acid sequence that is at least 80%, at least 85%, at least 90%, or at least 95% identical, or is 100% identical, to an amino acid sequence according to SEQ ID NO: 170.

Embodiment 55. A polyribonucleotide encoding one or more monkeypox antigens or fragments thereof, wherein the one or more monkeypox antigens or fragments thereof comprises an A35R amino acid sequence that is at least 80%, at least 85%, at least 90%, or at least 95% identical, or is 100% identical, to an amino acid sequence according to SEQ ID NO: 172.

Embodiment 56. A polyribonucleotide encoding one or more monkeypox antigens or fragments thereof, wherein the one or more monkeypox antigens or fragments thereof comprises an A35R amino acid sequence that is at least 80%, at least 85%, at least 90%, or at least 95% identical, or is 100% identical, to an amino acid sequence according to SEQ ID NO: 174.

Embodiment 57. A polyribonucleotide encoding one or more monkeypox antigens or fragments thereof, wherein the one or more monkeypox antigens or fragments thereof comprises (i) a first A35R amino acid sequence that is at least 80%, at least 85%, at least 90%, or at least 95% identical, or is 100% identical, to an amino acid sequence according to SEQ ID NO: 174 and (ii) a second A35R amino acid sequence that is at least 80%, at least 85%, at least 90%, or at least 95% identical, or is 100% identical, to an amino acid sequence according to SEQ ID NO: 174,

Embodiment 58. A polyribonucleotide encoding one or more monkeypox antigens or fragments thereof, wherein the first and second A35R amino acid sequences are linked by a linker, optionally wherein the linker has a sequence according to SEQ ID NO: 176.

Embodiment 59. A polyribonucleotide encoding one or more monkeypox antigens or fragments thereof, wherein the A35R amino acid sequence is operably linked with a signal peptide.

Embodiment 60. A polyribonucleotide encoding one or more monkeypox antigens or fragments thereof, wherein the signal peptide has an amino acid sequence according to SEQ ID NO: 160.

Embodiment 61. A polyribonucleotide encoding one or more monkeypox antigens or fragments thereof, wherein the polyribonucleotide encodes an antigen construct having an amino acid sequence that is at least 80%, at least 85%, at least 90%, or at least 95% identical, or is 100% identical, to an amino acid sequence according to SEQ ID NO: 178.

Embodiment 62. A polyribonucleotide encoding one or more monkeypox antigens or fragments thereof, wherein the one or more monkeypox antigens or fragments thereof comprises an B6 amino acid sequence that is at least 80%, at least 85%, at least 90%, or at least 95% identical, or is 100% identical, to an amino acid sequence according to SEQ ID NO: 180.

Embodiment 63. A polyribonucleotide encoding one or more monkeypox antigens or fragments thereof, wherein the one or more monkeypox antigens or fragments thereof comprises an B6 amino acid sequence that is at least 80%, at least 85%, at least 90%, or at least 95% identical, or is 100% identical, to an amino acid sequence according to SEQ ID NO: 182.

Embodiment 64. A polyribonucleotide encoding one or more monkeypox antigens or fragments thereof, wherein the one or more monkeypox antigens or fragments thereof comprises an H3L amino acid sequence that is at least 80%, at least 85%, at least 90%, or at least 95% identical, or is 100% identical, to an amino acid sequence according to SEQ ID NO: 184 and/or at least 80%, at least 85%, at least 90%, or at least 95% identical, or is 100% identical, to an amino acid sequence according to SEQ ID NO: 260.

Embodiment 65. A polyribonucleotide encoding one or more monkeypox antigens or fragments thereof, wherein the H3L amino acid sequence is operably linked with a signal peptide.

Embodiment 66. A polyribonucleotide encoding one or more monkeypox antigens or fragments thereof, wherein the signal peptide has an amino acid sequence according to SEQ ID NO: 160.

Embodiment 67. A polyribonucleotide encoding one or more monkeypox antigens or fragments thereof, wherein the polyribonucleotide encodes an antigen construct having an amino acid sequence that is at least 80%, at least 85%, at least 90%, or at least 95% identical, or is 100% identical, to an amino acid sequence according to SEQ ID NO: 186.

Embodiment 68. A polyribonucleotide encoding one or more monkeypox antigens or fragments thereof, wherein the one or more monkeypox antigens or fragments thereof comprises an H3L amino acid sequence that is at least 80%, at least 85%, at least 90%, or at least 95% identical, or is 100% identical, to an amino acid sequence according to SEQ ID NO: 188 and/or at least 80%, at least 85%, at least 90%, or at least 95% identical, or is 100% identical, to an amino acid sequence according to SEQ ID NO: 262.

Embodiment 69. A polyribonucleotide encoding one or more monkeypox antigens or fragments thereof, wherein the H3L amino acid sequence is operably linked with a signal peptide.

Embodiment 70. A polyribonucleotide encoding one or more monkeypox antigens or fragments thereof, wherein the signal peptide has an amino acid sequence according to SEQ ID NO: 160.

Embodiment 71. A polyribonucleotide encoding one or more monkeypox antigens or fragments thereof, wherein the polyribonucleotide encodes an antigen construct having an amino acid sequence that is at least 80%, at least 85%, at least 90%, or at least 95% identical, or is 100% identical, to an amino acid sequence according to SEQ ID NO: 190.

Embodiment 72. A polyribonucleotide encoding one or more monkeypox antigens or fragments thereof, wherein the one or more monkeypox antigens or fragments thereof comprises an E8L amino acid sequence that is (i) at least 80%, at least 85%, at least 90%, or at least 95% identical, or is 100% identical, to an amino acid sequence according to SEQ ID NO: 192, (ii) at least 80%, at least 85%, at least 90%, or at least 95% identical, or is 100% identical, to an amino acid sequence according to SEQ ID NO: 264, and/or (iii) and/or at least 80%, at least 85%, at least 90%, or at least 95% identical, or is 100% identical, to an amino acid sequence according to SEQ ID NO: 267.

Embodiment 73. A polyribonucleotide encoding one or more monkeypox antigens or fragments thereof, wherein the E8L amino acid sequence is operably linked with a signal peptide.

Embodiment 74. A polyribonucleotide encoding one or more monkeypox antigens or fragments thereof, wherein the signal peptide has an amino acid sequence according to SEQ ID NO: 160.

Embodiment 75. A polyribonucleotide encoding one or more monkeypox antigens or fragments thereof, wherein the polyribonucleotide encodes an antigen construct having an amino acid sequence that is at least 80%, at least 85%, at least 90%, or at least 95% identical, or is 100% identical, to an amino acid sequence according to SEQ ID NO: 194.

Embodiment 76. A polyribonucleotide encoding one or more monkeypox antigens or fragments thereof, wherein the one or more monkeypox antigens or fragments thereof comprises an A28L amino acid sequence that is at least 80%, at least 85%, at least 90%, or at least 95% identical, or is 100% identical, to an amino acid sequence according to SEQ ID NO: 196.

Embodiment 77. A polyribonucleotide encoding one or more monkeypox antigens or fragments thereof, wherein the A28L amino acid sequence is operably linked with a signal peptide.

Embodiment 78. A polyribonucleotide encoding one or more monkeypox antigens or fragments thereof, wherein the signal peptide has an amino acid sequence according to SEQ ID NO: 160.

Embodiment 79. A polyribonucleotide encoding one or more monkeypox antigens or fragments thereof, wherein the polyribonucleotide encodes an antigen construct having an amino acid sequence that is at least 80%, at least 85%, at least 90%, or at least 95% identical, or is 100% identical, to an amino acid sequence according to SEQ ID NO: 198.

Embodiment 80. A polyribonucleotide encoding one or more monkeypox antigens or fragments thereof, wherein the one or more monkeypox antigens or fragments thereof comprises (i) an A28L amino acid sequence that is at least 80%, at least 85%, at least 90%, or at least 95% identical, or is 100% identical, to an amino acid sequence according to SEQ ID NO: 196 and (ii) an A29L amino acid sequence that is at least 80%, at least 85%, at least 90%, or at least 95% identical, or is 100% identical, to an amino acid sequence according to SEQ ID NO: 171.

Embodiment 81. A polyribonucleotide encoding one or more monkeypox antigens or fragments thereof, wherein the polynucleotide encodes a signal peptide operably linked with the A28L amino acid sequence.

Embodiment 82. A polyribonucleotide encoding one or more monkeypox antigens or fragments thereof, wherein the polynucleotide encodes a signal peptide operably linked with the the A29L amino acid sequence.

Embodiment 83. A polyribonucleotide encoding one or more monkeypox antigens or fragments thereof, wherein the signal peptide has an amino acid sequence according to SEQ ID NO: 160.

Embodiment 84. A polyribonucleotide encoding one or more monkeypox antigens or fragments thereof, wherein the polyribonucleotide encodes an antigen construct having an amino acid sequence that is at least 80%, at least 85%, at least 90%, or at least 95% identical, or is 100% identical, to an amino acid sequence according to SEQ ID NO: 203.

Embodiment 85. A polyribonucleotide encoding one or more monkeypox antigens or fragments thereof, wherein the one or more monkeypox antigens or fragments thereof comprises a monkeypox A45L or a fragment thereof.

Embodiment 86. A polyribonucleotide encoding one or more monkeypox antigens or fragments thereof, wherein the monkeypox A45L or fragment thereof comprises an amino acid sequence that is at least 80%, at least 85%, at least 90%, or at least 95% identical to an amino acid sequence according to one of SEQ ID NOs: 61-65 (Table 2) or a portion thereof.

Embodiment 87. A polyribonucleotide encoding one or more monkeypox antigens or fragments thereof, wherein the monkeypox A45L or fragment thereof comprises or consists of an amino acid sequence that is identical to an amino acid sequence according to one of SEQ ID NOs: 61-65 (Table 2) or a corresponding portion thereof.

Embodiment 88. A polyribonucleotide encoding one or more monkeypox antigens or fragments thereof, wherein the one or more monkeypox antigens or fragments thereof comprises a monkeypox B9R or a fragment thereof.

Embodiment 89. A polyribonucleotide encoding one or more monkeypox antigens or fragments thereof, wherein the monkeypox B9R or fragment thereof comprises an amino acid sequence that is at least 80%, at least 85%, at least 90%, or at least 95% identical to an amino acid sequence according to one of SEQ ID NOs: 66-75 (Table 2) or a portion thereof.

Embodiment 90. A polyribonucleotide encoding one or more monkeypox antigens or fragments thereof, wherein the monkeypox B9R or fragment thereof comprises or consists of an amino acid sequence that is identical to an amino acid sequence according to one of SEQ ID NOs: 66-75 (Table 2) or a corresponding portion thereof.

Embodiment 91. A polyribonucleotide encoding one or more monkeypox antigens or fragments thereof, wherein the one or more monkeypox antigens or fragments thereof comprises a monkeypox B16R or a fragment thereof.

Embodiment 92. A polyribonucleotide encoding one or more monkeypox antigens or fragments thereof, wherein the monkeypox B16R or fragment thereof comprises an amino acid sequence that is at least 80%, at least 85%, at least 90%, or at least 95% identical to an amino acid sequence according to one of SEQ ID NOs: 76-85 (Table 2) or a portion thereof.

Embodiment 93. A polyribonucleotide encoding one or more monkeypox antigens or fragments thereof, wherein the monkeypox B16R or fragment thereof comprises or consists of an amino acid sequence that is identical to an amino acid sequence according to one of SEQ ID NOs: 76-85 (Table 2) or a corresponding portion thereof.

Embodiment 94. A polyribonucleotide encoding one or more monkeypox antigens or fragments thereof, wherein the one or more monkeypox antigens or fragments thereof comprises a monkeypox C10L or a fragment thereof.

Embodiment 95. A polyribonucleotide encoding one or more monkeypox antigens or fragments thereof, wherein the monkeypox C10L or fragment thereof comprises an amino acid sequence that is at least 80%, at least 85%, at least 90%, or at least 95% identical to an amino acid sequence according to one of SEQ ID NOs: 86-91 (Table 2) or a portion thereof.

Embodiment 96. A polyribonucleotide encoding one or more monkeypox antigens or fragments thereof, wherein the monkeypox C10L or fragment thereof comprises or consists of an amino acid sequence that is identical to an amino acid sequence according to one of SEQ ID NOs: 86-91 (Table 2) or a corresponding portion thereof.

Embodiment 97 A polyribonucleotide encoding one or more monkeypox antigens or fragments thereof, wherein the one or more monkeypox antigens or fragments thereof comprises a monkeypox C21L or a fragment thereof.

Embodiment 98. A polyribonucleotide encoding one or more monkeypox antigens or fragments thereof, wherein the monkeypox C21L or fragment thereof comprises an amino acid sequence that is at least 80%, at least 85%, at least 90%, or at least 95% identical to an amino acid sequence according to one of SEQ ID NOs: 92-96 (Table 2) or a portion thereof.

Embodiment 99. A polyribonucleotide encoding one or more monkeypox antigens or fragments thereof, wherein the monkeypox C21L or fragment thereof comprises or consists of an amino acid sequence that is identical to an amino acid sequence according to one of SEQ ID NOs: 92-96 (Table 2) or a corresponding portion thereof.

Embodiment 100. A polyribonucleotide encoding one or more monkeypox antigens or fragments thereof, wherein the one or more monkeypox antigens or fragments thereof comprises a monkeypox E7R or a fragment thereof.

Embodiment 101. A polyribonucleotide encoding one or more monkeypox antigens or fragments thereof, wherein the monkeypox E7R or fragment thereof comprises an amino acid sequence that is at least 80%, at least 85%, at least 90%, or at least 95% identical to an amino acid sequence according to one of SEQ ID NOs: 97-101 (Table 2) or a portion thereof.

Embodiment 102. A polyribonucleotide encoding one or more monkeypox antigens or fragments thereof, wherein the monkeypox E7R or fragment thereof comprises or consists of an amino acid sequence that is identical to an amino acid sequence according to one of SEQ ID NOs: 97-101 (Table 2) or a corresponding portion thereof.

Embodiment 103. A polyribonucleotide encoding one or more monkeypox antigens or fragments thereof, wherein the one or more monkeypox antigens or fragments thereof comprises a monkeypox F3L or a fragment thereof.

Embodiment 104. A polyribonucleotide encoding one or more monkeypox antigens or fragments thereof, wherein the monkeypox F3L or fragment thereof comprises an amino acid sequence that is at least 80%, at least 85%, at least 90%, or at least 95% identical to an amino acid sequence according to one of SEQ ID NOs: 102-111 (Table 2) or a portion thereof.

Embodiment 105. A polyribonucleotide encoding one or more monkeypox antigens or fragments thereof, wherein the monkeypox F3L or fragment thereof comprises or consists of an amino acid sequence that is identical to an amino acid sequence according to one of SEQ ID NOs: 102-111 (Table 2) or a corresponding portion thereof.

Embodiment 106. A polyribonucleotide encoding one or more monkeypox antigens or fragments thereof, wherein the one or more monkeypox antigens or fragments thereof comprises a monkeypox F4L or a fragment thereof.

Embodiment 107. A polyribonucleotide encoding one or more monkeypox antigens or fragments thereof, wherein the monkeypox F4L or fragment thereof comprises an amino acid sequence that is at least 80%, at least 85%, at least 90%, or at least 95% identical to an amino acid sequence according to one of SEQ ID NOs: 112-116 (Table 2) or a portion thereof.

Embodiment 108. A polyribonucleotide encoding one or more monkeypox antigens or fragments thereof, wherein the monkeypox F4L or fragment thereof comprises or consists of an amino acid sequence that is identical to an amino acid sequence according to one of SEQ ID NOs: 112-116 (Table 2) or a corresponding portion thereof.

Embodiment 109. A polyribonucleotide encoding one or more monkeypox antigens or fragments thereof, wherein the one or more monkeypox antigens or fragments thereof comprises a monkeypox G6R or a fragment thereof.

Embodiment 110. A polyribonucleotide encoding one or more monkeypox antigens or fragments thereof, wherein the monkeypox G6R or fragment thereof comprises an amino acid sequence that is at least 80%, at least 85%, at least 90%, or at least 95% identical to an amino acid sequence according to one of SEQ ID NOs: 117-121 (Table 2) or a portion thereof.

Embodiment 111. A polyribonucleotide encoding one or more monkeypox antigens or fragments thereof, wherein the monkeypox G6R or fragment thereof comprises or consists of an amino acid sequence that is identical to an amino acid sequence according to one of SEQ ID NOs: 117-121 (Table 2) or a corresponding portion thereof.

Embodiment 112. A polyribonucleotide encoding one or more monkeypox antigens or fragments thereof, wherein the one or more monkeypox antigens or fragments thereof comprises a monkeypox H5R or a fragment thereof.

Embodiment 113. A polyribonucleotide encoding one or more monkeypox antigens or fragments thereof, wherein the monkeypox H5R or fragment thereof comprises an amino acid sequence that is at least 80%, at least 85%, at least 90%, or at least 95% identical to an amino acid sequence according to one of SEQ ID NOs: 122-126 (Table 2) or a portion thereof.

Embodiment 114. A polyribonucleotide encoding one or more monkeypox antigens or fragments thereof, wherein the monkeypox H5R or fragment thereof comprises or consists of an amino acid sequence that is identical to an amino acid sequence according to one of SEQ ID NOs: 122-126 (Table 2) or a corresponding portion thereof.

Embodiment 115. A polyribonucleotide encoding one or more monkeypox antigens or fragments thereof, wherein the one or more monkeypox antigens or fragments thereof comprises a monkeypox I3L or a fragment thereof.

Embodiment 116. A polyribonucleotide encoding one or more monkeypox antigens or fragments thereof, wherein the monkeypox I3L or fragment thereof comprises an amino acid sequence that is at least 80%, at least 85%, at least 90%, or at least 95% identical to an amino acid sequence according to one of SEQ ID NOs: 127-131 (Table 2) or a portion thereof.

Embodiment 117. A polyribonucleotide encoding one or more monkeypox antigens or fragments thereof, wherein the monkeypox I3L or fragment thereof comprises or consists of an amino acid sequence that is identical to an amino acid sequence according to one of SEQ ID NOs: 127-131 (Table 2) or a corresponding portion thereof.

Embodiment 118. A polyribonucleotide encoding one or more monkeypox antigens or fragments thereof, wherein the one or more monkeypox antigens or fragments thereof comprises a monkeypox O2L or a fragment thereof.

Embodiment 119. A polyribonucleotide encoding one or more monkeypox antigens or fragments thereof, wherein the monkeypox O2L or fragment thereof comprises an amino acid sequence that is at least 80%, at least 85%, at least 90%, or at least 95% identical to an amino acid sequence according to one of SEQ ID NOs: 132-136 (Table 2) or a portion thereof.

Embodiment 120. A polyribonucleotide encoding one or more monkeypox antigens or fragments thereof, wherein the monkeypox O2L or fragment thereof comprises or consists of an amino acid sequence that is identical to an amino acid sequence according to one of SEQ ID NOs: 132-136 (Table 2) or a corresponding portion thereof.

Embodiment 121. A polyribonucleotide encoding one or more monkeypox antigens or fragments thereof, wherein the one or more monkeypox antigens or fragments thereof comprises a monkeypox Q1L or a fragment thereof.

Embodiment 122. A polyribonucleotide encoding one or more monkeypox antigens or fragments thereof, wherein the monkeypox Q1L or fragment thereof comprises an amino acid sequence that is at least 80%, at least 85%, at least 90%, or at least 95% identical to an amino acid sequence according to one of SEQ ID NOs: 137-141 (Table 2) or a portion thereof.

Embodiment 123. A polyribonucleotide encoding one or more monkeypox antigens or fragments thereof, wherein the monkeypox Q1L or fragment thereof comprises or consists of an amino acid sequence that is identical to an amino acid sequence according to one of SEQ ID NOs: 137-141 (Table 2) or a corresponding portion thereof.

Embodiment 124. A polyribonucleotide encoding one or more monkeypox antigens or fragments thereof, wherein the one or more monkeypox antigens or fragments thereof comprises a monkeypox B12R or a fragment thereof.

Embodiment 125. A polyribonucleotide encoding one or more monkeypox antigens or fragments thereof, wherein the monkeypox B12R or fragment thereof comprises an amino acid sequence that is at least 80%, at least 85%, at least 90%, or at least 95% identical to an amino acid sequence according to one of SEQ ID NOs: 244-247 (Table 2) or a portion thereof.

Embodiment 126. A polyribonucleotide encoding one or more monkeypox antigens or fragments thereof, wherein the monkeypox B12R or fragment thereof comprises or consists of an amino acid sequence that is identical to an amino acid sequence according to one of SEQ ID NOs: 244-247 (Table 2) or a corresponding portion thereof.

Embodiment 127. A polyribonucleotide encoding one or more monkeypox antigens or fragments thereof, wherein the one or more monkeypox antigens or fragments thereof comprises a monkeypox C17L or a fragment thereof.

Embodiment 128. A polyribonucleotide encoding one or more monkeypox antigens or fragments thereof, wherein the monkeypox C17L or fragment thereof comprises an amino acid sequence that is at least 80%, at least 85%, at least 90%, or at least 95% identical to an amino acid sequence according to one of SEQ ID NOs: 248-251 (Table 2) or a portion thereof.

Embodiment 129. A polyribonucleotide encoding one or more monkeypox antigens or fragments thereof, wherein the monkeypox C17L or fragment thereof comprises or consists of an amino acid sequence that is identical to an amino acid sequence according to one of SEQ ID NOs: 248-251 (Table 2) or a corresponding portion thereof.

Embodiment 130. A polyribonucleotide encoding one or more monkeypox antigens or fragments thereof, wherein the polyribonucleotide encodes two or more monkeypox antigens or fragments thereof as a single polypeptide.

Embodiment 131. A polyribonucleotide encoding one or more monkeypox antigens or fragments thereof, wherein the single polypeptide further comprises a linker.

Embodiment 132. A polyribonucleotide encoding one or more monkeypox antigens or fragments thereof, wherein the linker has a sequence comprising one or more glycine (G) residues and/or one or more serine (S) residues.

Embodiment 133. A polyribonucleotide encoding one or more monkeypox antigens or fragments thereof, wherein the linker is a cleavable linker.

Embodiment 134. A polyribonucleotide encoding one or more monkeypox antigens or fragments thereof, wherein the linker has a sequence found herein.

Embodiment 135. A polyribonucleotide encoding one or more monkeypox antigens or fragments thereof, wherein the one or more monkeypox antigens or fragments thereof comprises a monkeypox A45L or a fragment thereof that comprises an amino acid sequence that is at least 80%, at least 85%, at least 90%, or at least 95% identical to an amino acid sequence according to SEQ ID NO: 204.

Embodiment 136. A polyribonucleotide encoding one or more monkeypox antigens or fragments thereof, wherein the one or more monkeypox antigens or fragments thereof comprises a monkeypox Q1L or a fragment thereof that comprises an amino acid sequence that is at least 80%, at least 85%, at least 90%, or at least 95% identical to an amino acid sequence according to SEQ ID NO: 206.

Embodiment 137. A polyribonucleotide encoding one or more monkeypox antigens or fragments thereof, wherein the one or more monkeypox antigens or fragments thereof comprises a monkeypox Q1L or a fragment thereof that comprises an amino acid sequence that is at least 80%, at least 85%, at least 90%, or at least 95% identical to an amino acid sequence according to SEQ ID NO: 208.

Embodiment 138. A polyribonucleotide encoding one or more monkeypox antigens or fragments thereof, wherein the one or more monkeypox antigens or fragments thereof comprises a monkeypox B12R or a fragment thereof that comprises an amino acid sequence that is at least 80%, at least 85%, at least 90%, or at least 95% identical to an amino acid sequence according to SEQ ID NO: 210.

Embodiment 139. A polyribonucleotide encoding one or more monkeypox antigens or fragments thereof, wherein the one or more monkeypox antigens or fragments thereof comprises a monkeypox C17L or a fragment thereof that comprises an amino acid sequence that is at least 80%, at least 85%, at least 90%, or at least 95% identical to an amino acid sequence according to SEQ ID NO: 212.

Embodiment 140. A polyribonucleotide encoding one or more monkeypox antigens or fragments thereof, wherein the one or more monkeypox antigens or fragments thereof comprises a monkeypox C17L or a fragment thereof that comprises an amino acid sequence that is at least 80%, at least 85%, at least 90%, or at least 95% identical to an amino acid sequence according to SEQ ID NO: 214.

Embodiment 141. A polyribonucleotide encoding one or more monkeypox antigens or fragments thereof, wherein the one or more monkeypox antigens or fragments thereof comprises a monkeypox I3L or a fragment thereof that comprises an amino acid sequence that is at least 80%, at least 85%, at least 90%, or at least 95% identical to an amino acid sequence according to SEQ ID NO: 216.

Embodiment 142. A polyribonucleotide encoding one or more monkeypox antigens or fragments thereof, wherein the one or more monkeypox antigens or fragments thereof are operably linked with a signal peptide.

Embodiment 143. A polyribonucleotide encoding one or more monkeypox antigens or fragments thereof, wherein the signal peptide has an amino acid sequence that is at least 80%, at least 85%, at least 90%, or at least 95% identical, or is 100% identical, to an amino acid sequence according to SEQ ID NO: 218.

Embodiment 144. A polyribonucleotide encoding one or more monkeypox antigens or fragments thereof, wherein the one or more monkeypox antigens or fragments thereof are operably linked with an MITD.

Embodiment 145. A polyribonucleotide encoding one or more monkeypox antigens or fragments thereof, wherein the MITD has an amino acid sequence that is at least 80%, at least 85%, at least 90%, or at least 95% identical, or is 100% identical, to an amino acid sequence according to SEQ ID NO: 219.

Embodiment 146. A polyribonucleotide encoding one or more monkeypox antigens or fragments thereof, wherein the polyribonucleotide encodes an antigen construct having an amino acid sequence that is at least 80%, at least 85%, at least 90%, or at least 95% identical, or is 100% identical, to an amino acid sequence according to SEQ ID NO: 220.

Embodiment 147. A polyribonucleotide encoding one or more monkeypox antigens or fragments thereof, wherein the polyribonucleotide encodes an antigen construct having an amino acid sequence that is at least 80%, at least 85%, at least 90%, or at least 95% identical, or is 100% identical, to an amino acid sequence according to SEQ ID NO: 221.

Embodiment 148. A polyribonucleotide encoding one or more monkeypox antigens or fragments thereof, wherein the polyribonucleotide comprises ribonucleic acid sequences encoding each of the one or more monkeypox antigens or fragments thereof, and wherein each ribonucleic acid sequence encoding a monkeypox antigen or fragment thereof is codon optimized for expression in a subject, optionally wherein the subject is a human.

Embodiment 149. A polyribonucleotide encoding one or more monkeypox antigens or fragments thereof, wherein the polyribonucleotide comprises a ribonucleic acid sequence encoding a secretion signal.

Embodiment 150. A polyribonucleotide encoding one or more monkeypox antigens or fragments thereof, wherein the secretion signal comprises a ribonucleic acid sequence according to one of SEQ ID NOS: 143-154 (Table 3).

Embodiment 151. A polyribonucleotide encoding one or more monkeypox antigens or fragments thereof, wherein the polyribonucleotide comprises one or more non-coding sequence elements.

Embodiment 152. A polyribonucleotide encoding one or more monkeypox antigens or fragments thereof, wherein the one or more non-coding sequence elements enhances RNA stability and/or translation efficiency.

Embodiment 153. A polyribonucleotide encoding one or more monkeypox antigens or fragments thereof, wherein the one or more non-coding sequence elements comprise at least one 3′ untranslated region (UTR), at least one 5′ UTR, a 5′-cap, 5′ cap analog, a polyadenine (polyA) tail, or combination thereof.

Embodiment 154. A polyribonucleotide encoding one or more monkeypox antigens or fragments thereof, wherein the polyA tail is or comprises a modified polyA sequence, preferably an interrupted polyA tail.

Embodiment 155. A polyribonucleotide encoding one or more monkeypox antigens or fragments thereof, wherein the interrupted polyA tail comprises 30 adenine nucleotides followed by 70 adenine nucleotides, wherein the 30 adenine nucleotides and 70 adenine nucleotides are separated by a linker sequence.

Embodiment 156. A polyribonucleotide encoding one or more monkeypox antigens or fragments thereof, wherein the polyA tail comprises at least 100 adenine nucleotides.

Embodiment 157. A polyribonucleotide encoding one or more monkeypox antigens or fragments thereof, wherein the polyA tail comprises or consists of a sequence that is at least 90% identical to SEQ ID NO: 268 or 285.

Embodiment 158. A polyribonucleotide encoding one or more monkeypox antigens or fragments thereof, wherein the at least one 3′-UTR is or comprises a first sequence from the “amino terminal enhancer of split” (AES) messenger RNA and a second sequence from the mitochondrial encoded 12S ribosomal RNA.

Embodiment 159. A polyribonucleotide encoding one or more monkeypox antigens or fragments thereof, wherein the at least one 3′ UTR comprises or consists of a nucleic acid sequence that is at least 90% identical to SEQ ID NO: 268 or 285.

Embodiment 160. A polyribonucleotide encoding one or more monkeypox antigens or fragments thereof, wherein the at least one 5′-UTR is or comprises a modified human alpha-globin 5′-UTR.

Embodiment 161. A polyribonucleotide encoding one or more monkeypox antigens or fragments thereof, wherein the 5′ UTR comprises or consists of a nucleic acid sequence that is at least 90% identical to SEQ ID NO: 155.

Embodiment 162. A polyribonucleotide encoding one or more monkeypox antigens or fragments thereof, wherein the 5′ cap analog is or comprises Cap0, a Cap1 or a Cap2.

Embodiment 163. A polyribonucleotide encoding one or more monkeypox antigens or fragments thereof, wherein the 5′-cap is (m₂^{7,3′-O})Gppp(m^{2′-O})ApG.

Embodiment 164. A polyribonucleotide encoding one or more monkeypox antigens or fragments thereof, wherein the polyribonucleotide comprises one or more modified ribonucleotides.

Embodiment 165. A polyribonucleotide encoding one or more monkeypox antigens or fragments thereof, wherein the one or more modified ribonucleotides comprise modified uridine residues.

Embodiment 166. A polyribonucleotide encoding one or more monkeypox antigens or fragments thereof, wherein the modified uridine residues comprise N1-methyl-pseudouridine.

Embodiment 167. A polyribonucleotide encoding one or more monkeypox antigens or fragments thereof, wherein the polyribonucleotide is a non-natural polyribonucleotide.

Embodiment 168. A polyribonucleotide encoding one or more monkeypox antigens or fragments thereof, wherein the polyribonucleotide is an engineered polyribonucleotide.

Embodiment 169. A polyribonucleotide encoding one or more monkeypox antigens or fragments thereof, wherein the polyribonucleotide is an isolated polyribonucleotide.

Embodiment 170. A polyribonucleotide encoding one or more monkeypox antigens or fragments thereof, wherein the polyribonucleotide is codon optimized for expression in a subject, optionally wherein the subject is a human.

Embodiment 171. A composition comprising a polyribonucleotide encoding one or more monkeypox antigens or fragments thereof.

Embodiment 172. A composition comprising a polyribonucleotide encoding one or more monkeypox antigens or fragments thereof, wherein the plurality of polyribonucleotides comprises at least two polyribonucleotides that are not the same.

Embodiment 173. A composition comprising a polyribonucleotide encoding one or more monkeypox antigens or fragments thereof, wherein at least one polyribonucleotide of the plurality of polyribonucleotides encodes one or more monkeypox antigens or fragments thereof.

Embodiment 174. The composition of claim 172 or 17 A composition comprising a polyribonucleotide encoding one or more monkeypox antigens or fragments thereof 3, wherein all of the polyribonucleotides of the plurality of polyribonucleotides encode one or more monkeypox antigens or fragments thereof.

Embodiment 175. A composition comprising a polyribonucleotide encoding one or more monkeypox antigens or fragments thereof, wherein at least one polyribonucleotide of the plurality of polyribonucleotides encodes only one monkeypox antigen or fragment thereof.

Embodiment 176. A composition comprising a polyribonucleotide encoding one or more monkeypox antigens or fragments thereof, wherein a first polyribonucleotide of the plurality of polyribonucleotides encodes a first set of one or more monkeypox antigens or fragments thereof, wherein the first set of one or more monkeypox antigens or fragments thereof comprise:
(i) B6R or a fragment of B6R,
(ii) M1R or a fragment of M1R,
(iii) A35R or a fragment of A35R,
(iv) H3L or a fragment of H3L, and
(v) E8L or a fragment of E8L, or
(vi) a combination of any thereof,
wherein a second polyribonucleotide of the plurality of polyribonucleotides encodes a second set of one or more monkeypox antigens or fragments thereof, wherein the first set of one or more monkeypox antigens or fragments thereof and the second set of one or more monkeypox antigens or fragments thereof are different.

Embodiment 177. A composition comprising a polyribonucleotide encoding one or more monkeypox antigens or fragments thereof, wherein a first polyribonucleotide of the plurality of polyribonucleotides encodes a first set of one or more monkeypox antigens or fragments thereof, wherein the first set of one or more monkeypox antigens or fragments thereof comprise:
(i) A29L or a fragment of A29L,
(ii) A35R or a fragment of A35R,
(iii) B6R or a fragment of B6R,
(iv) M1R or a fragment of M1R,
(v) E8L or a fragment of E8L,
(vi) A28L or a fragment of A28L,
(vii) H3L or a fragment of H3L, or
(viii) a combination thereof;
wherein a second polyribonucleotide of the plurality of polyribonucleotides encodes a second set of one or more monkeypox antigens or fragments thereof, wherein the first set of one or more monkeypox antigens or fragments thereof and the second set of one or more monkeypox antigens or fragments thereof are different.

Embodiment 178. A composition comprising a polyribonucleotide encoding one or more monkeypox antigens or fragments thereof, wherein a first polyribonucleotide of the plurality of polyribonucleotides encodes a first set of one or more monkeypox antigens or fragments thereof, wherein the first set of one or more monkeypox antigens or fragments thereof comprise:
(i) A45L or a fragment of A45L,
(ii) B9R or a fragment of B9R,
(iii) B16R or a fragment of B16R,
(iv) C10L or a fragment of C10L,
(v) C21L or a fragment of C21L,
(vi) E7R or a fragment of E7R,
(vii) F3L or a fragment of F3L,
(viii) F4L or a fragment of F4L,
(ix) G6R or a fragment of G6R,
(x) H5R or a fragment of H5R,
(xi) I3L or a fragment of I3L,
(xii) O2L or a fragment of O2L,
(xiii) Q1L or a fragment of Q1L,
(xiv) B12R or a fragment of B12R,
(xv) C17L or a fragment of C17L, or
(xvi) a combination thereof;
wherein a second polyribonucleotide of the plurality of polyribonucleotides encodes a second set of one or more monkeypox antigens or fragments thereof, wherein the first set of one or more monkeypox antigens or fragments thereof and the second set of one or more monkeypox antigens or fragments thereof are different.

Embodiment 179. A composition comprising a polyribonucleotide encoding one or more monkeypox antigens or fragments thereof, wherein a first polyribonucleotide of the plurality of polyribonucleotides encodes a first set of one or more monkeypox antigens or fragments thereof, wherein the first set of one or more monkeypox antigens or fragments thereof comprise:
(i) A29L or a fragment of A29L,
(ii) A35R or a fragment of A35R,
(iii) B6R or a fragment of B6R,
(iv) M1R or a fragment of M1R,
(v) E8L or a fragment of E8L,
(vi) H3L or a fragment of H3L,
(vii) A45L or a fragment of A45L,
(viii) B9R or a fragment of B9R,
(ix) B16R or a fragment of B16R,
(x) C10L or a fragment of C10L,
(xi) C21L or a fragment of C21L,
(xii) E7R or a fragment of E7R,
(xiii) F3L or a fragment of F3L,
(xiv) F4L or a fragment of F4L,
(xv) G6R or a fragment of G6R,
(xvi) H5R or a fragment of H5R,
(xvii) I3L or a fragment of I3L,
(xviii) O2L or a fragment of O2L,
(xix) Q1L or a fragment of Q1L,
(xx) B12R or a fragment of B12R,
(xxi) C17L or a fragment of C17L,
(xxii) A28L or a fragment of A28L, or
(xxiii) a combination thereof,
wherein a second polyribonucleotide of the plurality of polyribonucleotides encodes a second set of one or more monkeypox antigens or fragments thereof, wherein the first set of one or more monkeypox antigens or fragments thereof and the second set of one or more monkeypox antigens or fragments thereof are different.

Embodiment 180. A composition comprising a polyribonucleotide encoding one or more monkeypox antigens or fragments thereof, wherein the first set of one or more monkeypox antigens or fragments thereof and the second set of one or more monkeypox antigens or fragments thereof do not include any of the same monkeypox antigens or fragments thereof.

Embodiment 181. A composition comprising a polyribonucleotide encoding one or more monkeypox antigens or fragments thereof, wherein the composition further comprises lipid nanoparticles, polyplexes (PLX), lipidated polyplexes (LPLX), or liposomes, wherein the polyribonucleotide or plurality of polyribonucleotides are fully or partially encapsulated within the lipid nanoparticles, polyplexes (PLX), lipidated polyplexes (LPLX), or liposomes.

Embodiment 182. A composition comprising a polyribonucleotide encoding one or more monkeypox antigens or fragments thereof, wherein the composition further comprises lipid nanoparticles, wherein the polyribonucleotide or plurality of polyribonucleotides are fully or partially encapsulated within the lipid nanoparticles.

Embodiment 183. A composition comprising a polyribonucleotide encoding one or more monkeypox antigens or fragments thereof, wherein the lipid nanoparticles target liver cells.

Embodiment 184. A composition comprising a polyribonucleotide encoding one or more monkeypox antigens or fragments thereof, wherein the lipid nanoparticles target secondary lymphoid organ cells.

Embodiment 185. A composition comprising a polyribonucleotide encoding one or more monkeypox antigens or fragments thereof, wherein the lipid nanoparticles are cationic lipid nanoparticles.

Embodiment 186. A composition comprising a polyribonucleotide encoding one or more monkeypox antigens or fragments thereof, wherein the lipid nanoparticles each comprise:
(a) a polymer-conjugated lipid;
(b) a cationic lipid; and
(c) one or more neutral lipids.

Embodiment 187. A composition comprising a polyribonucleotide encoding one or more monkeypox antigens or fragments thereof, wherein the polymer-conjugated lipid comprises a PEG-conjugated lipid.

Embodiment 188. A composition comprising a polyribonucleotide encoding one or more monkeypox antigens or fragments thereof, wherein the polymer-conjugated lipid comprises 2-[(polyethylene glycol)-2000]—N,N-ditetradecylacetamide.

Embodiment 189. A composition comprising a polyribonucleotide encoding one or more monkeypox antigens or fragments thereof, wherein the one or more neutral lipids comprise 1,2-Distearoyl-sn-glycero-3-phosphocholine (DPSC).

Embodiment 190. A composition comprising a polyribonucleotide encoding one or more monkeypox antigens or fragments thereof, wherein the one or more neutral lipids comprise cholesterol.

Embodiment 191. A composition comprising a polyribonucleotide encoding one or more monkeypox antigens or fragments thereof, wherein the cationic lipid comprises ((3-hydroxypropyl)azanediyl)bis(nonane-9,1-diyl)bis(2-butyloctanoate).

Embodiment 192. A composition comprising a polyribonucleotide encoding one or more monkeypox antigens or fragments thereof, wherein the lipid nanoparticles each comprise:
(a) 2-[(polyethylene glycol)-2000]—N,N-ditetradecylacetamide;
(b) DPSC;
(c) cholesterol; and
(d) ((3-hydroxypropyl)azanediyl)bis(nonane-9,1-diyl)bis(2-butyloctanoate).

Embodiment 193. A composition comprising a polyribonucleotide encoding one or more monkeypox antigens or fragments thereof, wherein the lipid nanoparticles comprise:
(a) the polymer-conjugated lipid at about 1-2.5 mol % of the total lipids;
(b) the cationic lipid at 35-65 mol % of the total lipids; and
(c) the one or more neutral lipids are present in 35-65 mol % of the total lipids.

Embodiment 194. A composition comprising a polyribonucleotide encoding one or more monkeypox antigens or fragments thereof, wherein the lipid nanoparticles have an average diameter of about 50-150 nm.

Embodiment 195. A composition comprising a polyribonucleotide encoding one or more monkeypox antigens or fragments thereof, wherein the total RNA is present in an amount within a range of 1 μg to 100 μg per dose in the composition.

Embodiment 196. A pharmaceutical composition comprising a composition comprising a polyribonucleotide encoding one or more monkeypox antigens or fragments thereof and at least one pharmaceutically acceptable excipient.

Embodiment 197. A pharmaceutical composition comprising a composition comprising a polyribonucleotide encoding one or more monkeypox antigens or fragments thereof, wherein the pharmaceutical comprises a cryoprotectant.

Embodiment 198. A pharmaceutical composition comprising a composition comprising a polyribonucleotide encoding one or more monkeypox antigens or fragments thereof, wherein the pharmaceutical comprises an aqueous buffered solution.

Embodiment 199. A combination comprising: a first polyribonucleotide encoding a B6R antigen or fragment thereof, and a second polyribonucleotide encoding an M1R antigen or fragment thereof.

Embodiment 200. A combination comprising: a first pharmaceutical composition comprising a first polyribonucleotide encoding a B6R antigen or fragment thereof, and a second pharmaceutical composition comprising a second polyribonucleotide encoding an M1R antigen or fragment thereof.

Embodiment 201. A combination comprising: a first polyribonucleotide encoding a B6R antigen or fragment thereof,
a second polyribonucleotide encoding an M1R antigen or fragment thereof, and
a third polyribonucleotide encoding a A35R antigen or fragment thereof.

Embodiment 202. A combination comprising:
a first pharmaceutical composition comprising a first polyribonucleotide encoding a B6R antigen or fragment thereof,
a second pharmaceutical composition comprising a second polyribonucleotide encoding an M1R antigen or fragment thereof, and
a third pharmaceutical composition comprising a third polyribonucleotide encoding an A35R antigen or fragment thereof.

Embodiment 203. A combination comprising:
a first polyribonucleotide encoding a B6R antigen or fragment thereof,
a second polyribonucleotide encoding an M1R antigen or fragment thereof,
a third polyribonucleotide encoding a A35R antigen or fragment thereof, and
a fourth polyribonucleotide encoding a E8L antigen or fragment thereof.

Embodiment 204. A combination comprising:
a first pharmaceutical composition comprising a first polyribonucleotide encoding a B6R antigen or fragment thereof,
a second pharmaceutical composition comprising a second polyribonucleotide encoding an M1R antigen or fragment thereof, and
a third pharmaceutical composition comprising a third polyribonucleotide encoding an A35R antigen or fragment thereof, and
a fourth pharmaceutical composition comprising a fourth polyribonucleotide encoding an E8L antigen or fragment thereof.

Embodiment 205. A combination comprising:
a first polyribonucleotide encoding a B6R antigen or fragment thereof,
a second polyribonucleotide encoding an M1R antigen or fragment thereof,
a third polyribonucleotide encoding a A35R antigen or fragment thereof, and
a fourth polyribonucleotide encoding a H3L antigen or fragment thereof.

Embodiment 206. A combination comprising:
a first pharmaceutical composition comprising a first polyribonucleotide encoding a B6R antigen or fragment thereof,
a second pharmaceutical composition comprising a second polyribonucleotide encoding an M1R antigen or fragment thereof, and
a third pharmaceutical composition comprising a third polyribonucleotide encoding an A35R antigen or fragment thereof, and
a fourth pharmaceutical composition comprising a fourth polyribonucleotide encoding an H3L antigen or fragment thereof.

Embodiment 207. A method comprising administering a pharmaceutical composition comprising a composition comprising a polyribonucleotide encoding one or more monkeypox antigens or fragments thereof to a subject.

Embodiment 208. A method comprising administering a combination comprising a first, second, third, and fourth pharmaceutical composition to a subject, wherein the first, second, third, and fourth pharmaceutical composition each comprise a polyribonucleotide encoding a different monkeypox antigen.

Embodiment 209. A pharmaceutical composition comprising a composition comprising a polyribonucleotide encoding one or more monkeypox antigens or fragments thereof for use in the treatment of monkeypox comprising administering the pharmaceutical composition to a subject.

Embodiment 210. A pharmaceutical composition comprising a composition comprising a polyribonucleotide encoding one or more monkeypox antigens or fragments thereof for use in the prevention of monkeypox comprising administering the pharmaceutical composition to a subject.

Embodiment 211. A pharmaceutical composition comprising a composition comprising a polyribonucleotide encoding one or more monkeypox antigens or fragments thereof, wherein administering the pharmaceutical composition to the subject comprises administering one or more doses of the pharmaceutical composition to the subject.

Embodiment 212. A pharmaceutical composition comprising a composition comprising a polyribonucleotide encoding one or more monkeypox antigens or fragments thereof, wherein the one or more doses of the pharmaceutical composition are administered to the subject weekly.

Embodiment 213. A pharmaceutical composition comprising a composition comprising a polyribonucleotide encoding one or more monkeypox antigens or fragments thereof, wherein the one or more doses of the pharmaceutical composition are administered to the subject bi-weekly.

Embodiment 214. A pharmaceutical composition comprising a composition comprising a polyribonucleotide encoding one or more monkeypox antigens or fragments thereof, wherein the pharmaceutical composition is administered intravenously.

Embodiment 215. A pharmaceutical composition comprising a composition comprising a polyribonucleotide encoding one or more monkeypox antigens or fragments thereof, wherein the pharmaceutical composition is administered intramuscularly.

Embodiment 216. A pharmaceutical composition comprising a composition comprising a polyribonucleotide encoding one or more monkeypox antigens or fragments thereof, wherein the pharmaceutical composition is administered subcutaneously.

Embodiment 217. A pharmaceutical composition comprising a composition comprising a polyribonucleotide encoding one or more monkeypox antigens or fragments thereof, wherein the subject has or is at risk of developing a monkeypox infection.

Embodiment 218. A method comprising administering a pharmaceutical composition comprising a composition comprising a polyribonucleotide encoding one or more monkeypox antigens or fragments thereof, wherein the method is a method of treating a monkeypox infection.

Embodiment 219. A method comprising administering a pharmaceutical composition comprising a composition comprising a polyribonucleotide encoding one or more monkeypox antigens or fragments thereof, wherein the method is a method of preventing a monkeypox infection.

Embodiment 220. Use of a pharmaceutical composition comprising a composition comprising a polyribonucleotide encoding one or more monkeypox antigens or fragments thereof for the treatment of monkeypox in a subject.

Embodiment 222. Use of a pharmaceutical composition comprising a composition comprising a polyribonucleotide encoding one or more monkeypox antigens or fragments thereof, wherein the subject has or is at risk of developing a monkeypox infection.

Embodiment 223. Use of a pharmaceutical composition comprising a composition comprising a polyribonucleotide encoding one or more monkeypox antigens or fragments thereof for the treatment of monkeypox in a subject.

Embodiment 224. Use of a pharmaceutical composition comprising a composition comprising a polyribonucleotide encoding one or more monkeypox antigens or fragments thereof for the prevention of monkeypox in a subject.

Embodiment 225. Use of a pharmaceutical composition comprising a composition comprising a polyribonucleotide encoding one or more monkeypox antigens or fragments thereof, wherein the subject has or is at risk of developing a monkeypox infection.

Embodiment A method of characterizing efficacy of a vaccine composition, comprising at least one polyribonucleotide encoding one or more monkeypox antigens or fragments thereof, the method comprising:
(i) administering the vaccine composition to a CAST/Ei mouse in one or more doses;
(ii) infecting the mouse with an orthopoxvirus; and
(iii) measuring a viral titer of the orthopoxvirus in a sample collected from the mouse,
wherein the vaccine composition is characterized as efficacious if the viral titer is significantly lower than a viral titer of the orthopoxvirus in a control sample.

Embodiment 227. A method of manufacturing a vaccine composition, comprising at least one polyribonucleotide encoding one or more monkeypox antigens or fragments thereof, the method comprising:
testing the ability of the vaccine composition to significantly lower an orthopoxvirus viral titer measured in a sample collected from a CAST/Ei mouse as compared to a control sample, wherein the CAST/Ei mouse was administered with the vaccine composition, in one or more doses prior to infection with the orthopoxvirus.

Embodiment 228. A method of characterizing efficacy of a vaccine composition, wherein a dose of the vaccine composition is administered to the CAST/Ei mouse 25 to 75 days prior to infection with the orthopoxvirus.

Embodiment 229. A method of characterizing efficacy of a vaccine composition, wherein the dose of the vaccine composition is administered to the CAST/Ei mouse 35 to 65 days prior to infection with the orthopoxvirus.

Embodiment 230. A method of characterizing efficacy of a vaccine composition, wherein the dose of the vaccine composition is administered to the CAST/Ei mouse 50 to 60 days prior to infection with the orthopoxvirus.

Embodiment 231. A method of characterizing efficacy of a vaccine composition, wherein the dose of the vaccine composition is administered to the CAST/Ei mouse 56 days prior to infection with the orthopoxvirus.

Embodiment 232. A method of characterizing efficacy of a vaccine composition, wherein the dose of the vaccine composition comprises 0.5 to 10 μg of the polyribonucleotide.

Embodiment 233. A method of characterizing efficacy of a vaccine composition, wherein the dose of the vaccine composition comprises 1 to 5 μg of the polyribonucleotide.

Embodiment 234. A method of characterizing efficacy of a vaccine composition, wherein the dose of the vaccine composition comprises 1 μg of the polyribonucleotide.

Embodiment 235. A method of characterizing efficacy of a vaccine composition, wherein the dose of the vaccine composition comprises 4 μg of the polyribonucleotide.

Embodiment 236. A method of characterizing efficacy of a vaccine composition, wherein the dose of the vaccine composition is administered to the mouse intramuscularly, intravenously, or intranasally.

Embodiment 237. A method of characterizing efficacy of a vaccine composition, wherein the dose of the vaccine composition is administered to the mouse intramuscularly.

Embodiment 238. A method of characterizing efficacy of a vaccine composition, wherein an additional dose of the vaccine composition is administered to the CAST/Ei mouse 20 to 50 days prior to infection with the orthopoxvirus.

Embodiment 239. A method of characterizing efficacy of a vaccine composition, wherein the additional dose of the vaccine composition is administered to the CAST/Ei mouse 30 to 40 days prior to infection with the orthopoxvirus.

Embodiment 240 A method of characterizing efficacy of a vaccine composition, wherein the additional dose of the vaccine composition is administered to the CAST/Ei mouse 32 to 38 days prior to infection with the orthopoxvirus.

Embodiment 241. A method of characterizing efficacy of a vaccine composition, wherein the additional dose of the vaccine composition is administered to the CAST/Ei mouse 35 days prior to infection with the orthopoxvirus.

Embodiment 242. A method of characterizing efficacy of a vaccine composition, wherein the additional dose of the vaccine composition comprises 0.5 to 10 g of the polyribonucleotide.

Embodiment 243. A method of characterizing efficacy of a vaccine composition, wherein the additional dose of the vaccine composition comprises 1 to 5 μg of the polyribonucleotide.

Embodiment 244. A method of characterizing efficacy of a vaccine composition, wherein the additional dose of the vaccine composition comprises 1 μg of the polyribonucleotide.

Embodiment 245. A method of characterizing efficacy of a vaccine composition, wherein the additional dose of the vaccine composition comprises 4 μg of the polyribonucleotide.

Embodiment 246. A method of characterizing efficacy of a vaccine composition, wherein the additional dose of the vaccine composition is administered to the mouse intramuscularly, intravenously, or intranasally.

Embodiment 247. A method of characterizing efficacy of a vaccine composition, wherein the dose of the vaccine composition is administered to the mouse intramuscularly.

Embodiment 248. A method of characterizing efficacy of a vaccine composition, wherein the CAST/Ei mouse is infected with 6×10⁶ PFU to 1.2×10⁷ PFU of the orthopoxvirus.

Embodiment 249. A method of characterizing efficacy of a vaccine composition, wherein the CAST/Ei mouse is infected with 7×10⁶ PFU to 1×10⁷ PFU of the orthopoxvirus.

Embodiment 250. A method of characterizing efficacy of a vaccine composition, wherein the CAST/Ei mouse is infected with 9.0×10⁶ PFU of the orthopoxvirus.

Embodiment 251. A method of characterizing efficacy of a vaccine composition, wherein the CAST/Ei mouse is intranasally infected with the orthopoxvirus.

Embodiment 252. A method of characterizing efficacy of a vaccine composition, wherein the sample is collected from the mouse 2 to 10 days post infection with the orthopoxvirus.

Embodiment 253. A method of characterizing efficacy of a vaccine composition, wherein the sample is collected from the mouse 3 days post infection with the orthopoxvirus.

Embodiment 254. A method of characterizing efficacy of a vaccine composition, wherein the sample is collected from the mouse 7 days post infection with the orthopoxvirus.

Embodiment 255. A method of characterizing efficacy of a vaccine composition, wherein the sample is a lung tissue sample, a bronchiolar lavage sample, or blood sample.

Embodiment 256. A method of characterizing efficacy of a vaccine composition, wherein the sample is a homogenized lung tissue sample.

Embodiment 257. A method of characterizing efficacy of a vaccine composition, wherein the control sample is a sample collected from a CAST/Ei mouse that has been infected with the orthopoxvirus but has not been administered with the vaccine composition.

Embodiment 258. A method of characterizing efficacy of a vaccine composition, wherein the orthopoxvirus is a monkeypox virus, vaccinia virus, variola virus, or cowpox virus.

Embodiment 259. A method of characterizing efficacy of a vaccine composition, wherein the viral titer of the orthopoxvirus in the sample collected from the mouse is significantly lower than the viral titer of the orthopoxvirus in the control sample, when the viral titer of the orthopoxvirus in the sample collected from the mouse is at least 40% lower than the viral titer of the orthopoxvirus in the control sample.

Embodiment 260. A method of characterizing efficacy of a vaccine composition, wherein the viral titer of the orthopoxvirus in the sample collected from the mouse is significantly lower than the viral titer of the orthopoxvirus in the control sample, when the viral titer of the orthopoxvirus in the sample collected from the mouse is at least 60% lower than the viral titer of the orthopoxvirus in the control sample.

Embodiment 261. A method of characterizing efficacy of a vaccine composition, wherein the viral titer of the orthopoxvirus in the sample collected from the mouse is significantly lower than the viral titer of the orthopoxvirus in the control sample, when the viral titer of the orthopoxvirus in the sample collected from the mouse is at least 60% lower than the viral titer of the orthopoxvirus in the control sample.

Embodiment 262. A method of characterizing efficacy of a vaccine composition, wherein the viral titer of the orthopoxvirus in the sample collected from the mouse is significantly lower than the viral titer of the orthopoxvirus in the control sample, when the viral titer of the orthopoxvirus in the sample collected from the mouse is at least 80% lower than the viral titer of the orthopoxvirus in the control sample.

Embodiment 263. A method of characterizing efficacy of a vaccine composition, wherein the viral titer of the orthopoxvirus in the sample collected from the mouse is significantly lower than the viral titer of the orthopoxvirus in the control sample, when the viral titer of the orthopoxvirus in the sample collected from the mouse is at least 90% lower than the viral titer of the orthopoxvirus in the control sample.

### EXAMPLES

### Example 1: Antigens for Monkeypox Vaccination

Exemplary datasets curated for identification of antigens and/or epitopes of monkeypox for vaccine compositions include, and approaches to the analysis thereof, include those discussed in the present Example.

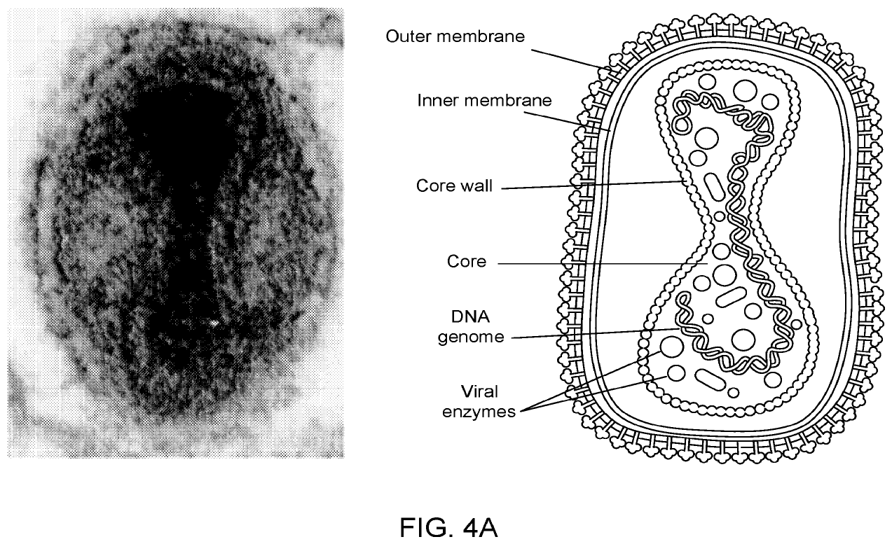

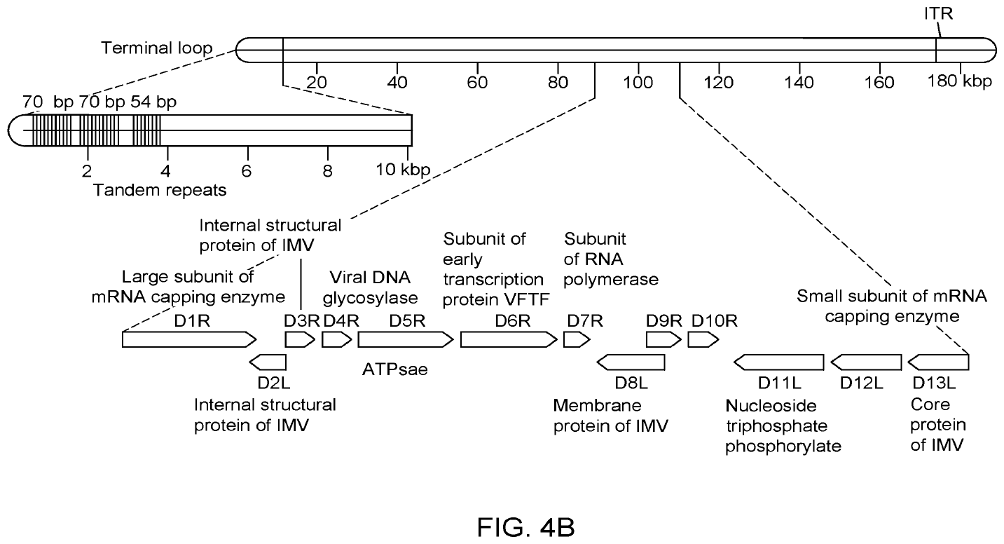

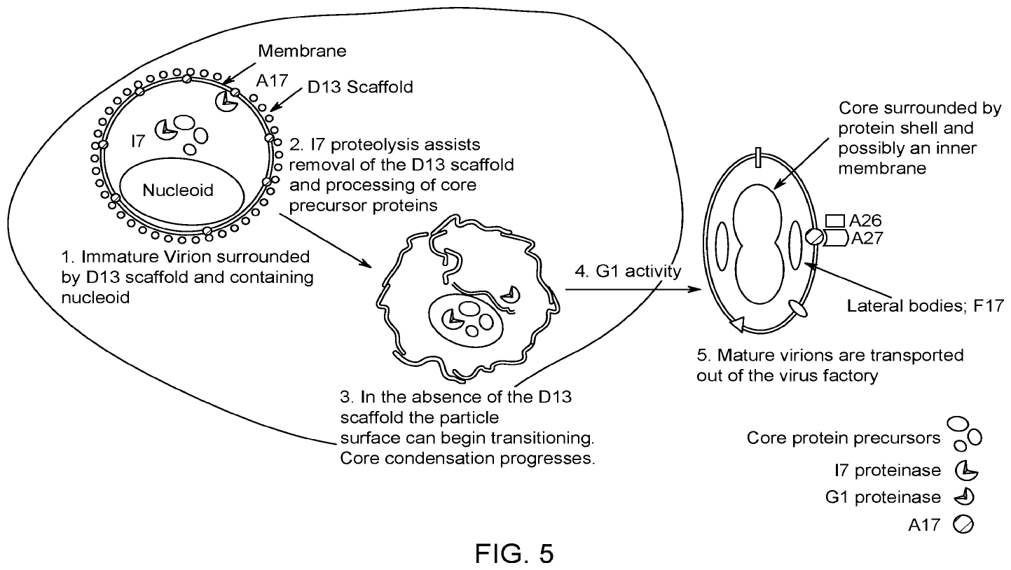

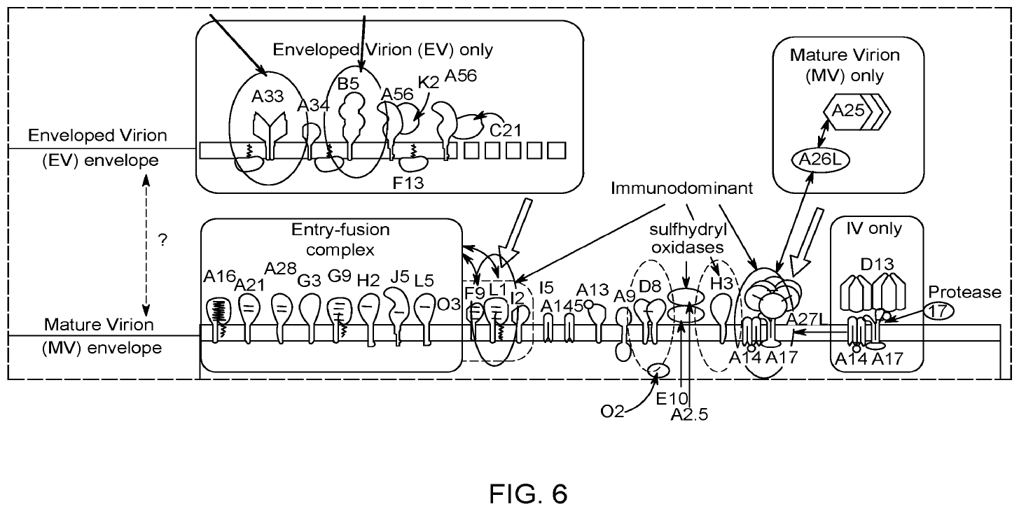

Monkeypox is a member of the poxvirus family and orthopoxvirus genus (FIG. 1 and FIG. 2). Orthopoxviruses are characterized by structural and lifecycle complexity (FIG. 3). The study of orthopoxviruses has typically focused on vaccinia virus (FIGS. 4A and 4B), which is a representative poxvirus that follows a typical poxvirus lifecycle (FIG. 5). Certain past studies have analyzed vaccines against orthopoxvirus such as vaccinia virus. The present disclosure includes the recognition that studies suggest that humoral B cell responses appear to be protective and/or necessary in vaccine efficiacy against other pox viruses. The present disclosure further includes the recognition that T cell responses have been observed, and often correlate with protection, but do not appear to be necessary for vaccine efficacy. Vaccinia virus vaccine data has shown that certain antigens can be useful, in certain contexts and formats, for vaccinia virus therapy. These have included vaccinia antigens A27L, A33R, B5R, and L1R (see, e.g., accession numbers AAN78218.2, AAF63733, AAN78219.1, and AAF63732, respectively, homologous to A29L, A35R, B6R, and M1R in monkeypox, exemplary but non-limiting sequences of which can canonically include the sequences of accession numbers QJQ40281.1, QJQ40286.1, AAN78223.1, and QJQ40223.1, respectively; see also FIG. 6). These antigens have been shown to confer at least partial protection when utilized individually, and significant or even complete protection when utilized in combination.

Vaccinia L1R is a myristoylated transmembrane protein of about 250 residues that is expressed on the surface of the IMVs (intracellular mature virions). It is considered essential at least in that genetic deletion redners vaccinia viruses incapable of maturation. L1R appears to be required for maturation of viral particles. See, e.g., DOIs: https://doi.org/10.1128/jvi.68.10.6401-6410.1994, 10.1073/pnas.062163799

Vaccinia A27L is implicated in virus attachment, virus-cell fusion, cell-cell-fusion, plaque size and the formation of enveloped virions, IMV-specific. See, e.g., DOIs: 10.1128/JVI.72.2.1577-1585.1998, https://doi.org/10.1128/jvi.64.10.4884-4892.1990, 10.1016/0042-6822(90)90381-z, 10.1016/0042-6822(87)90481-8, 10.1099/0022-1317-32-1-63

Vaccinia A33R is a type II integral membrane protein found in EEV (extracellular enveloped virus) but not IMV, and is highly conserved among orthopoxviruses. See, e.g., DOIs: 10.1128/jvi.72.5.4192-4204.1998

B5R is a membrane protein that is essential in packaging the intracellular mature virion form intracellular enveloped virions, and is EEV-specific. DOIs: https://doi.org/10.1083/jcb.200104124, https://doi.org/10.1128/jvi.68.1.130-147.1994, https://doi.org/10.1099/0022-1317-83-12-2915

In the present example, to identify antigen sequences useful in the preparation of compositions (e.g., vaccines) as disclosed herein, isolates from recent outbreaks of monkeypox were selected. In particular, sequences from two isolates were used to determine consensus sequences for antigens of interest (Portugal 20220503: https://virological.org/t/first-draft-genome-sequence-of-monkeypox-virus-associated-with-the-suspected-multi-country-outbreak-may-2022-confirmed-case-in-portugal/799, and Belgium_20220513: https://virological.org/t/belgian-case-of-monkeypox-virus-linked-to-outbreak-in-portugal/801). Antigens were then identified by identifying homology with previously identified monkeypox strains. Antigen sequences were aligned, and a consensus sequence was obtained using the Belgian sequence (which has much greater coverage) and filling in sequencing ambiguities with the corresponding Portugal sequences. No discordant residues were identified between the two isolates.

By way of context, a number of vaccinia virus vaccines have been attempted. Historically, vaccines produced according to certain platforms for pox virus vaccination such as polypeptide-based vaccines have not been satisfactory. For example, ACAM2000 (Sanofi Pasteur) is a live-attenuated vaccinia virus cultured in Vero cells. Approved in 2007, ACAM2000 replaced Dryvax. As an infectious vaccine, ACAM2000 is dangerous to immune-deficient and at-risk individuals. Another example is JYNNEOS (Bavarian Nordic AS), which is a live-attenuated non replicating vaccinia virus that can be used with relatively greater safety in immune-deficient individuals. Another vaccinia vaccine known as VIg (Vaccinia Immunoglobulin) includes a mixture of antibodies for passive vaccination in immune-deficient individuals, but has not been tested in humans.

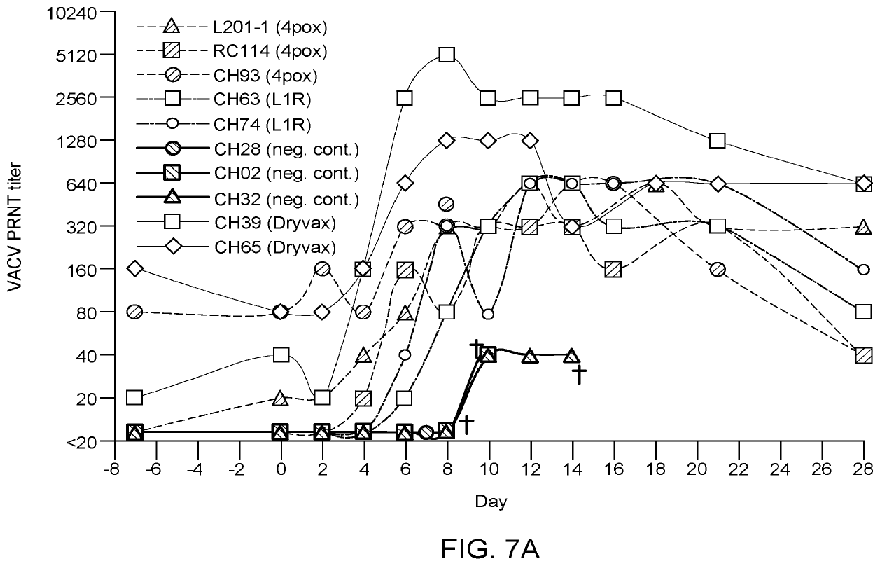

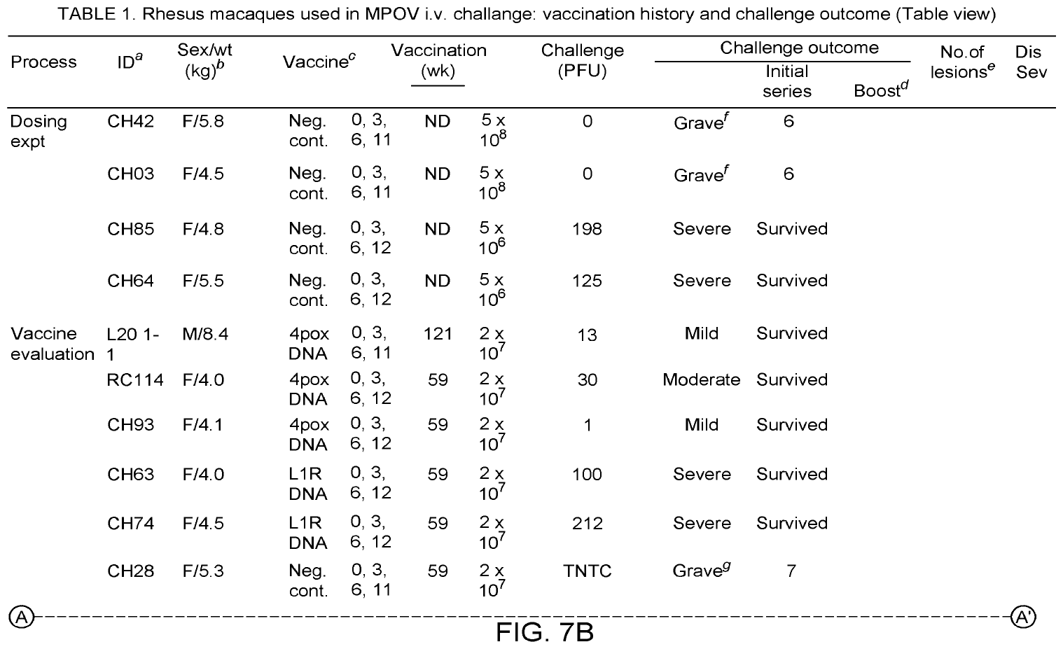

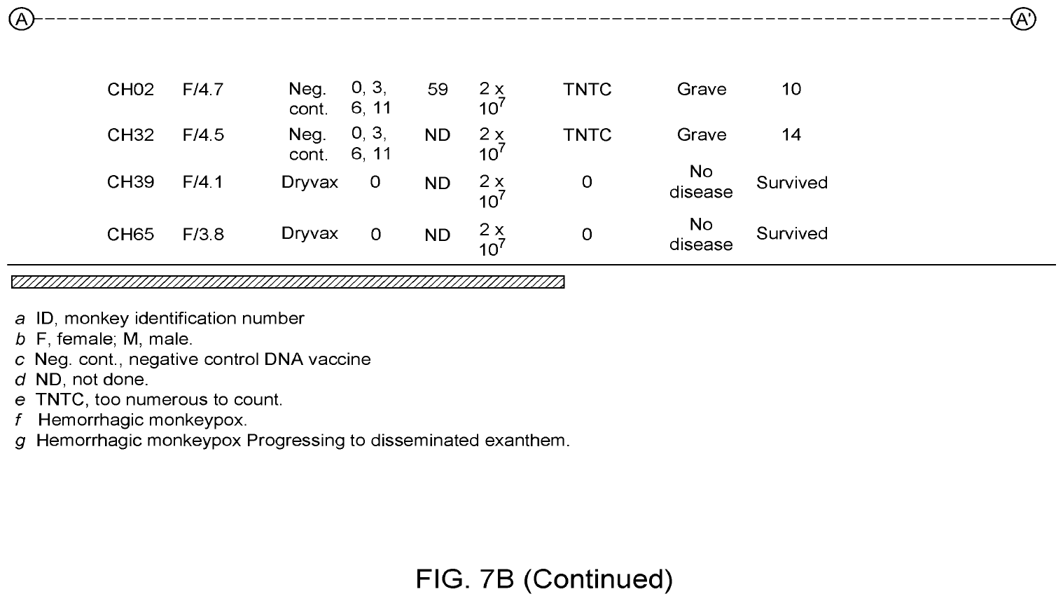

Hooper et al., JVI 2004 (https://doi.org/10.1128/JVI.78.9.4433-4443.2004 “Smallpox DNA Vaccine Protects Nonhuman Primates against Lethal Monkeypox”) demonstrated that a DNA vaccine with up to four antigens provides partial protection againast monkeypox in Rehsus macaques. Although live attenuated (Dryvax) vaccine had superior protection in terms of complete disease prevention, Dryvax is associated with certain limitations discussed above. Vaccines including 4 antigens (4-pox) showed a greater reduction in disease severity than vaccines including a single antigen (1-pox), although the quality of DNA vaccine antibody was low (high titer, low neutralization). Data from Hooper et al. is provided in FIGS. 7A and 7B.

Fogg et al. (DOI: 10.1128/JVI.78.19.10230-10237.2004; “Protective Immunity to Vaccinia Virus Induced by Vaccination with Multiple Recombinant Outer Membrane Proteins of Intracellular and Extracellular Virions”) utilized recombinant protein immunization with three antigens, in particular A33, B5, and L1. Immunization with these antigens protected mice immunized with live vaccinia virus. Mice immunized with a single recombinant antigen protein or combinations were resistant to challenge with vaccinia virus. Mice immunized with A33+B5+L1 or with A33+L1 were better protected than mice immunized with live virus. Data from Fogg et al. is provided in FIGS. 8A and 8B.

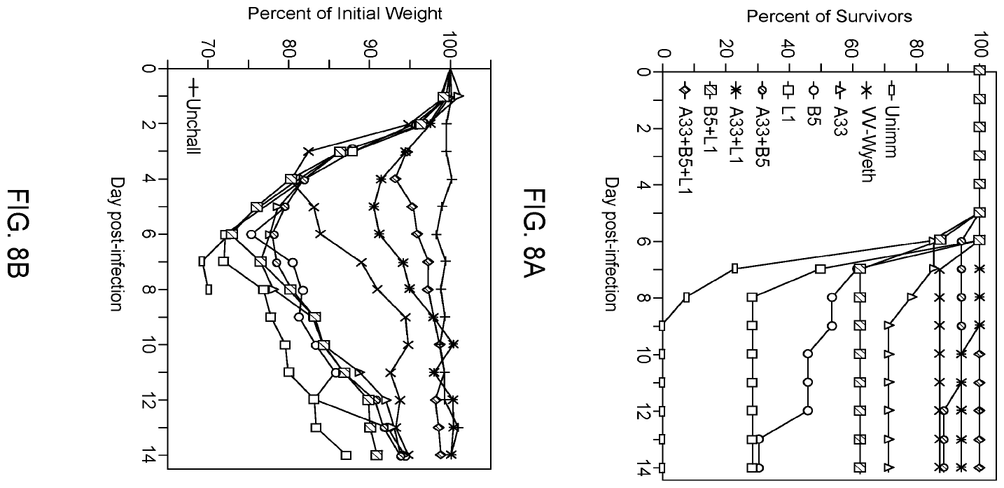

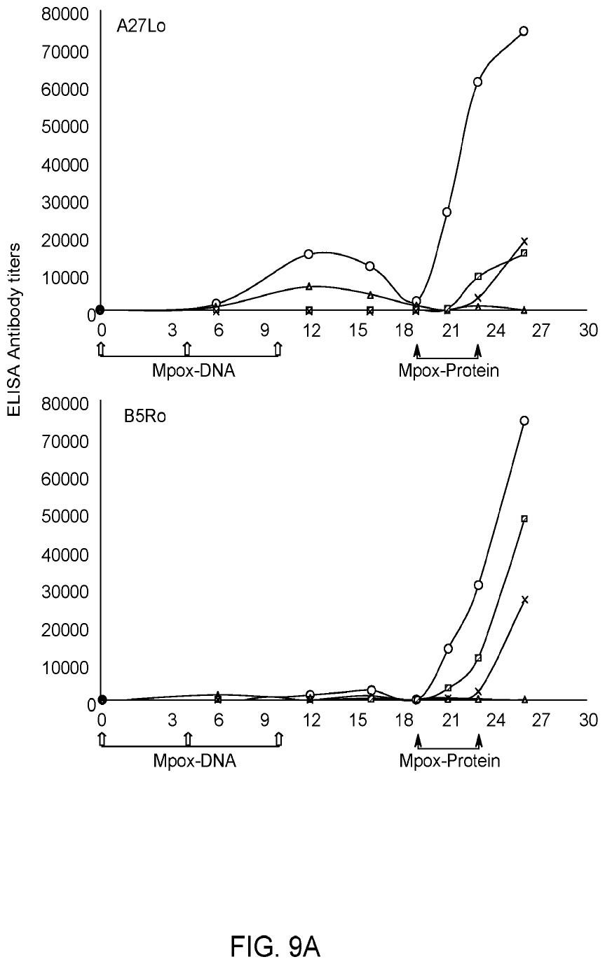

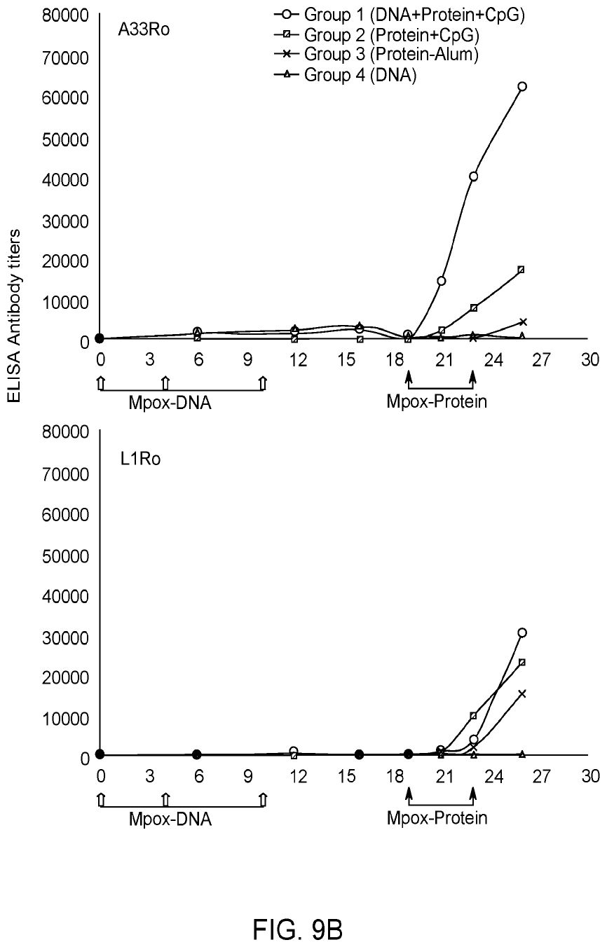

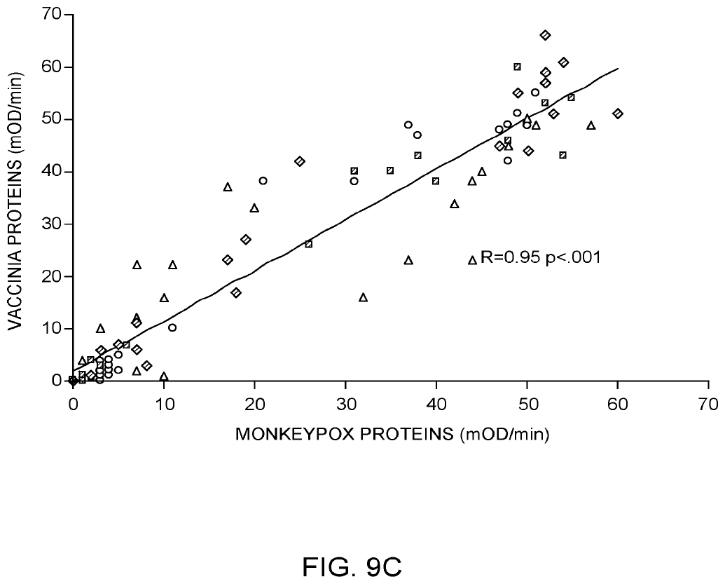

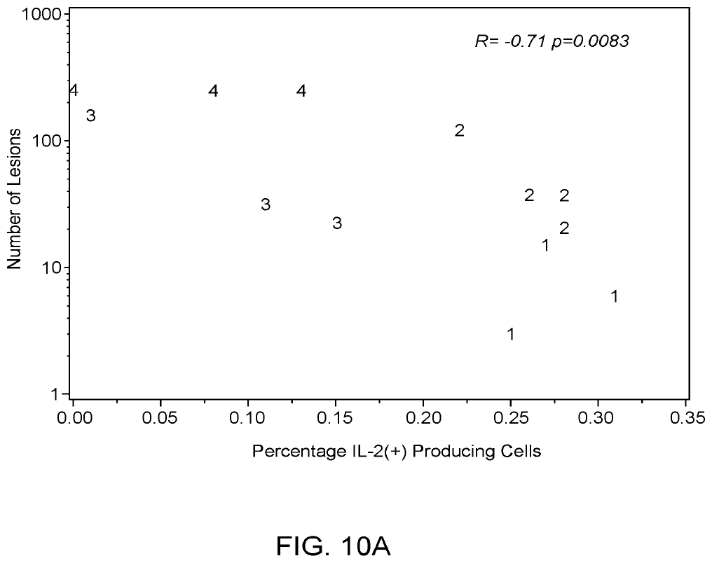

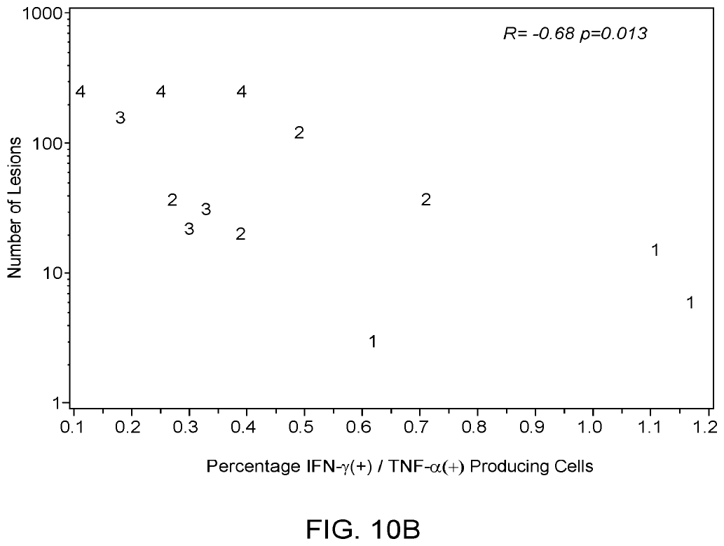

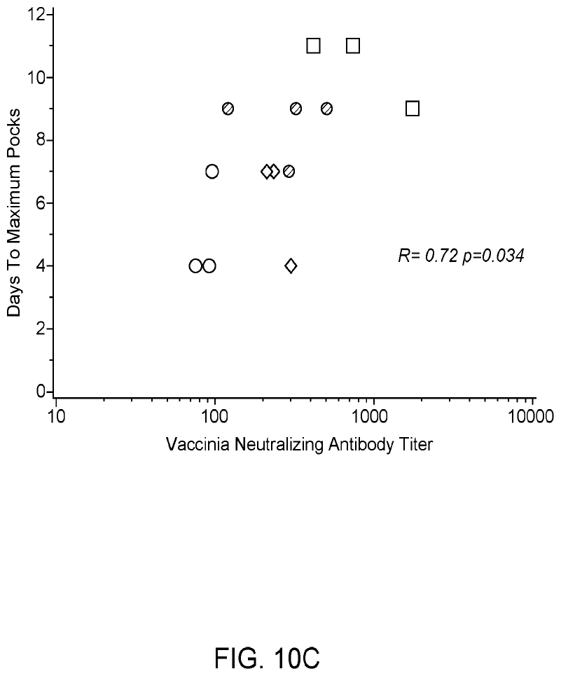

Heraud et al. (https://doi.org/10.4049/jimmunol.177.4.2552; “Subunit Recombinant Vaccine Protects against Monkeypox”) demonstrated that DNA and recombinant protein immunization of Rehsus macaques using antigens L1, A27, A33, and B5 reduces lesion number and delays disease manifestation. Data from Heraud et al. is shown in FIGS. 9A-9C and FIGS. 10A-10C.

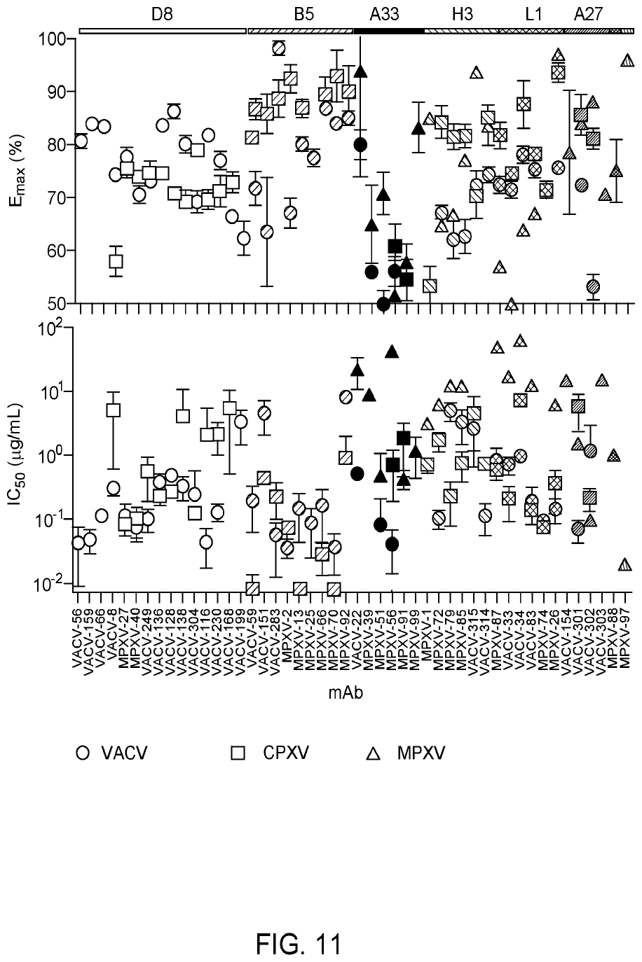

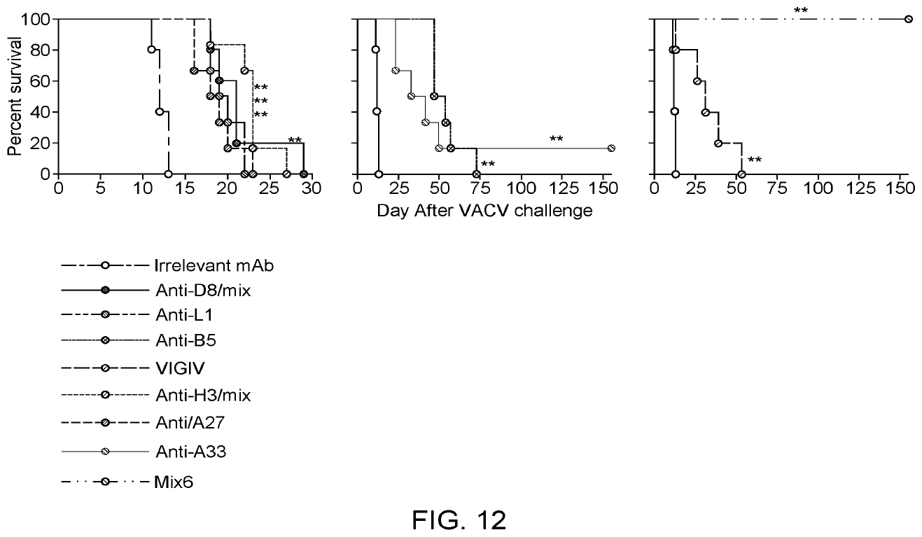

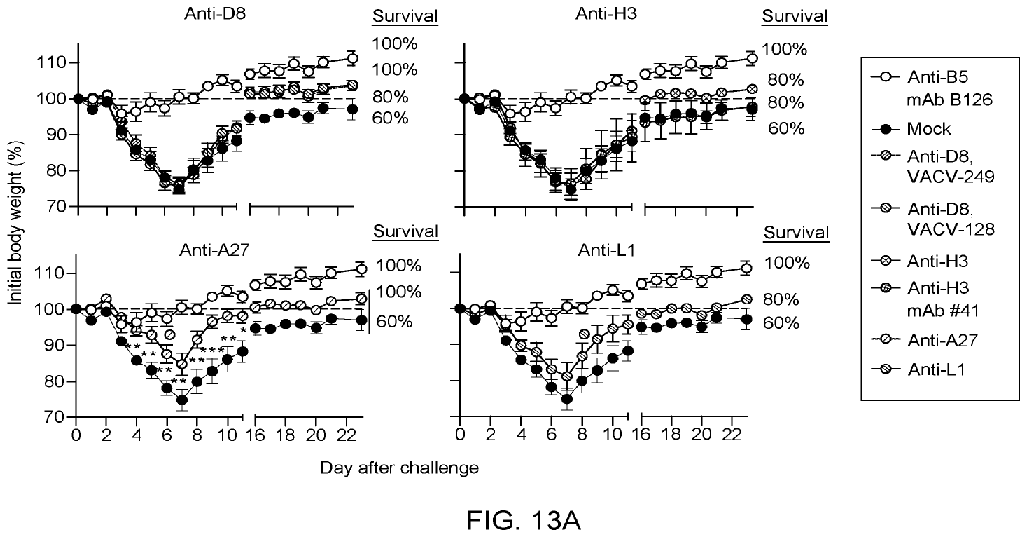

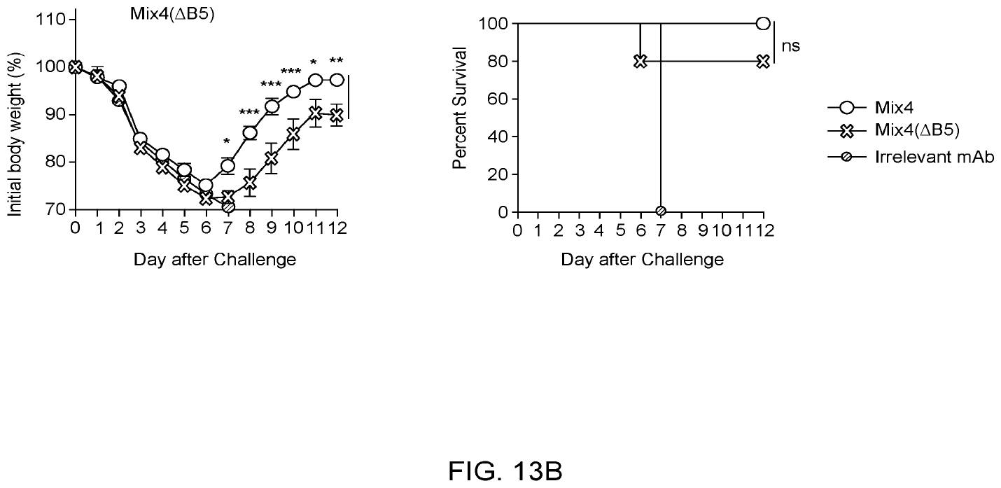

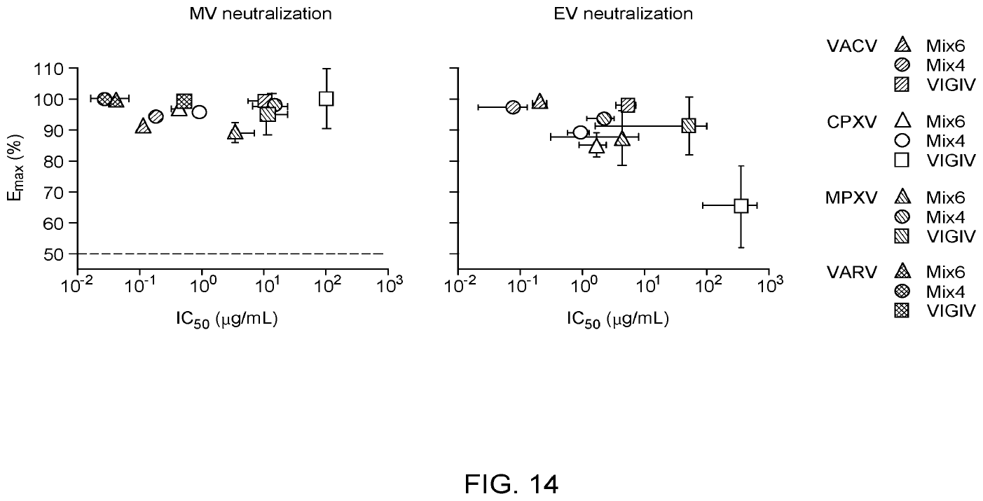

Gilchuk et al Cell 2016 (DOI: 10.1016/j.cell.2016.09.049; “Cross-Neutralizing and Protective Human Antibody Specificities to Poxvirus Infections”) demonstrated that pox virus entry is complex, with many envelope proteins that could be targeted/involved in neutralization. A large panel of monoclonals was isolated from vaccinia immunized healthcare workers. Neutralizing monoclonal antibodies against D8, B5, A33, H3, L1 and A27 were identified, of which B5 monoclonals appeasr to have the most preferable profile. Combinatiosn of monoclonal antibodies provided protection in mouse studies. Related date from Gilchuk et al. is shown in FIG. 11 and FIG. 12. Gilchuk et al. demonstrated that combination of four mAbs targeting different targets also prevented disease where individual monoclonals fail. Combination of 3 monoclonals can still prevent death (but not weight loss). In vitro neutralization with 4 mAbs and 6 mAbs produced similar results across all tested viruses (VACV, CPXV, MPXV, VARV). Related date from Gilchuk et al. is shown in FIGS. 13A-13B and FIG. 14.

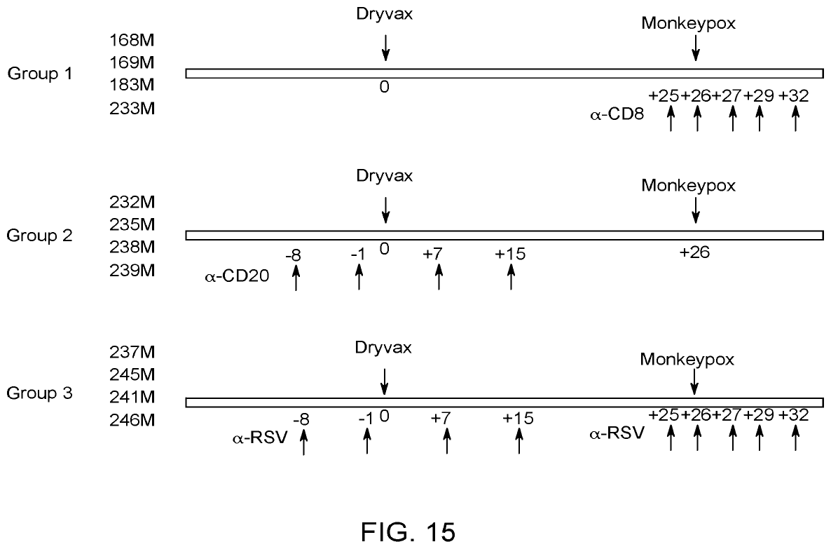

In a study of Dryvax by Edghill-Smith et al. (Nature Medicine 2005 (https://doi.org/10.1038/nm1261)), it was shown that B cell depletion but not T cell depletion abrogated Dryvax protection. Data from Edghill-Smith et al is shown in FIG. 15 and FIG. 16.

The present disclosure includes, for example, compositions (e.g., antigen constructs and/or vaccines) that include and/or encode one or more, and preferably all of, monkeypox antigens A29L, A35R, B6R, and MIR, or fragments thereof. Additional vaccinia antigens of unknown relative contributions to vaccinia vaccination efforts include vaccinia H3 and D8. To the knowledge of the present inventors, it has not been demonstrated or previously predicted that certain of the antigens and/or combinations of antigens provided herein are particularly effective for treatment (e.g., vaccination against) monkeypox and/or for use in compositions and methods in which one or more of the antigens provided herein are encoded by a polyribonucleotide.

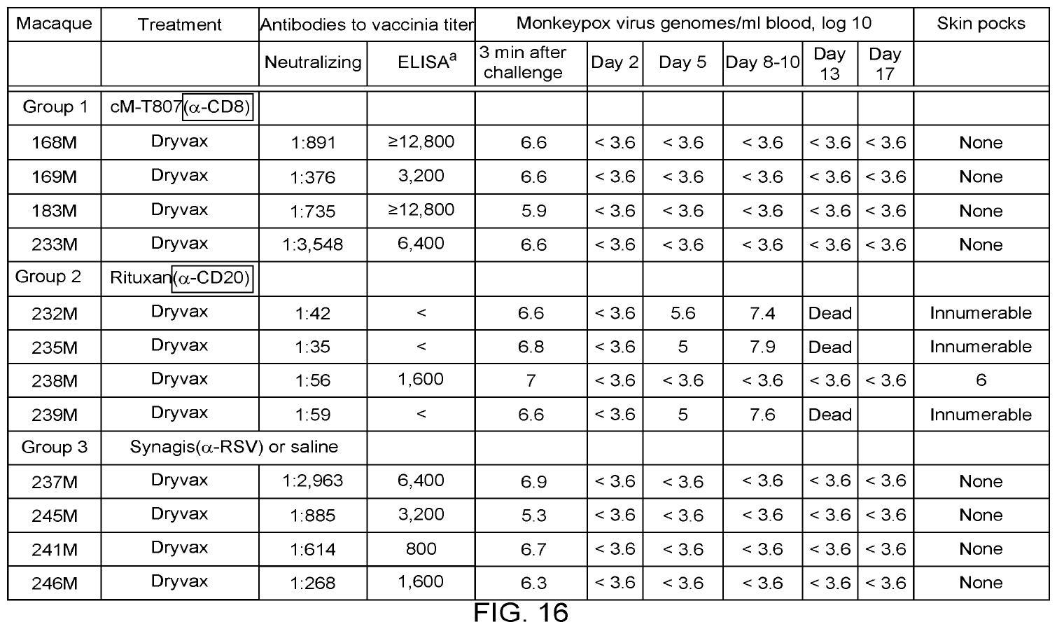

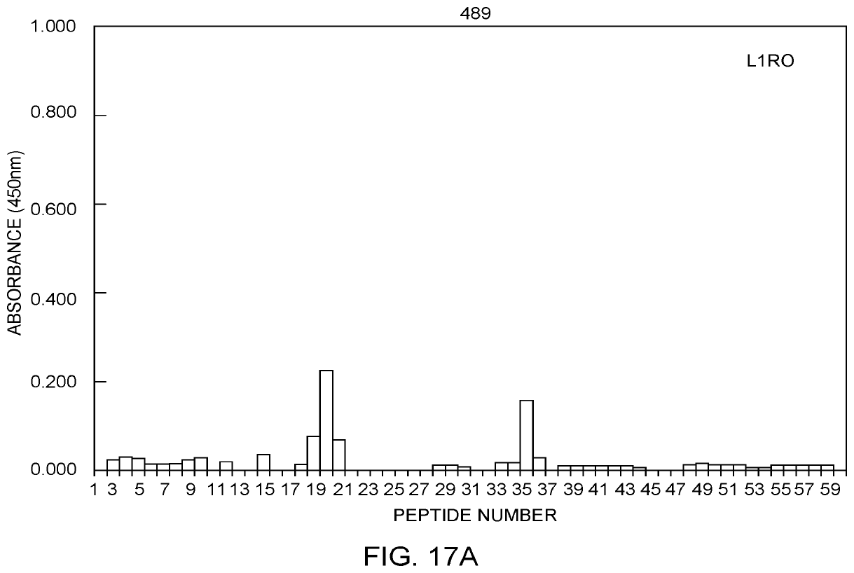

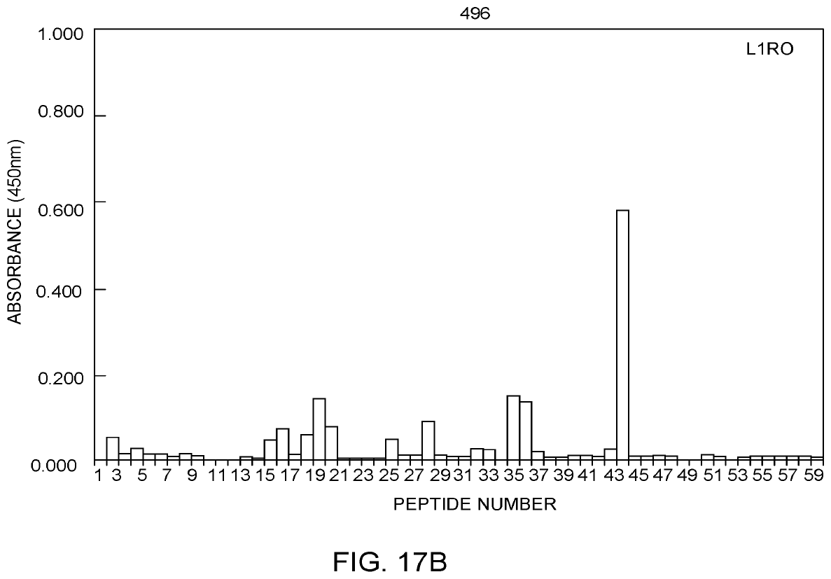

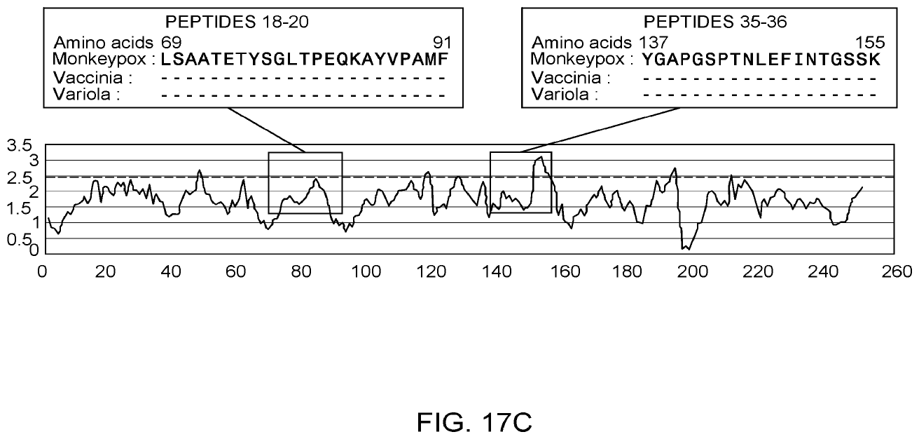

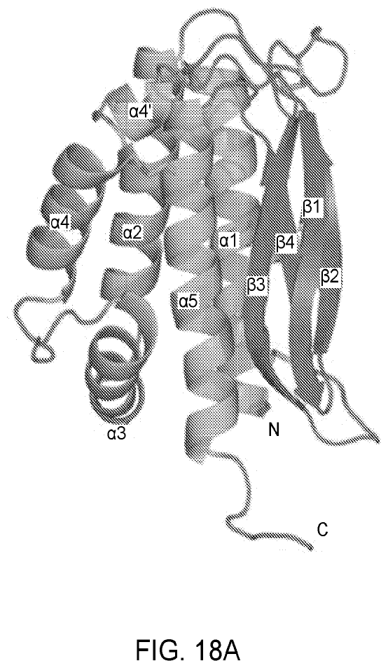

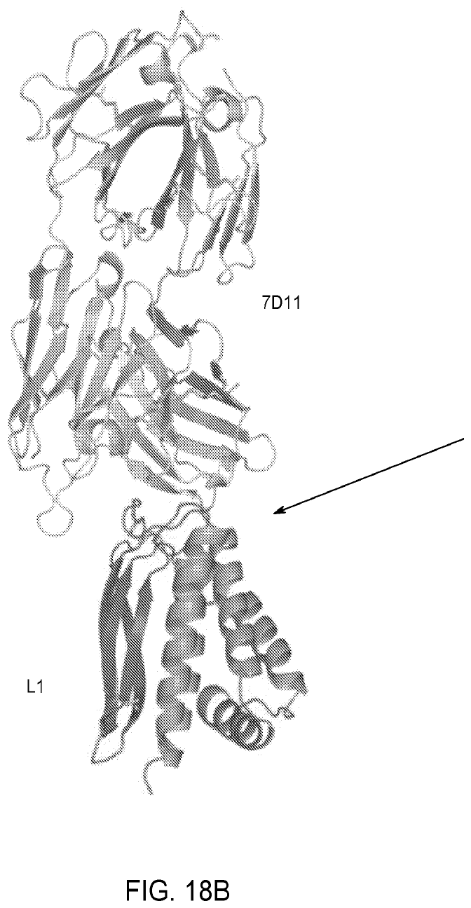

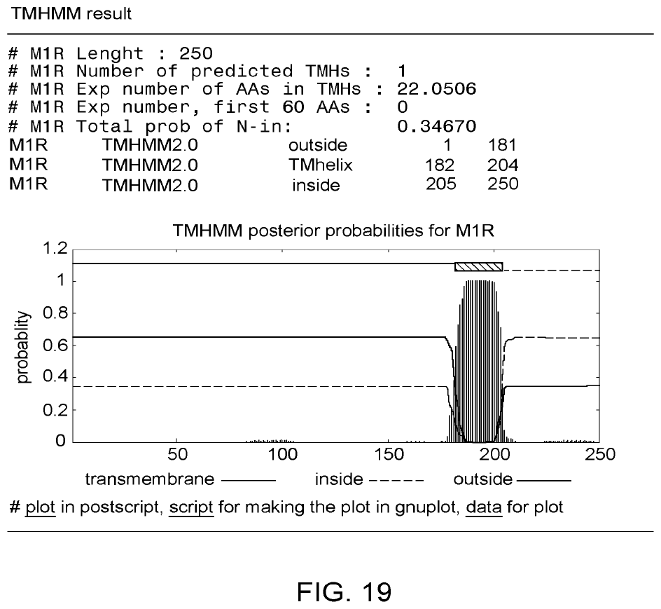

Monkeypox MIR was found to bear high sequence similarity to vaccinia L1R. In particular, MPX MIR derived from GenBank AY160187.1 and LIR derived from NP_042117.1 are 98.8% identical on the amino acid level. Heraud et al. (https://doi.org/10.4049/jimmunol.177.4.2552) provide L1R B cell epitope mapping by ELISA, analyzing linear epitopes (FIG. 17A-17C). The structure of SPX L1R from Su et al. 2005. PNAS. is shown in FIGS. 18A and 18B, together with a prediction of transmembrane helices (FIG. 19).

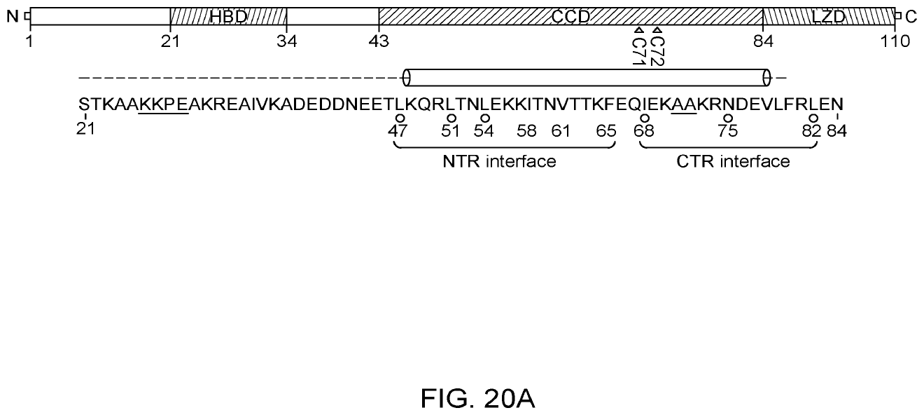

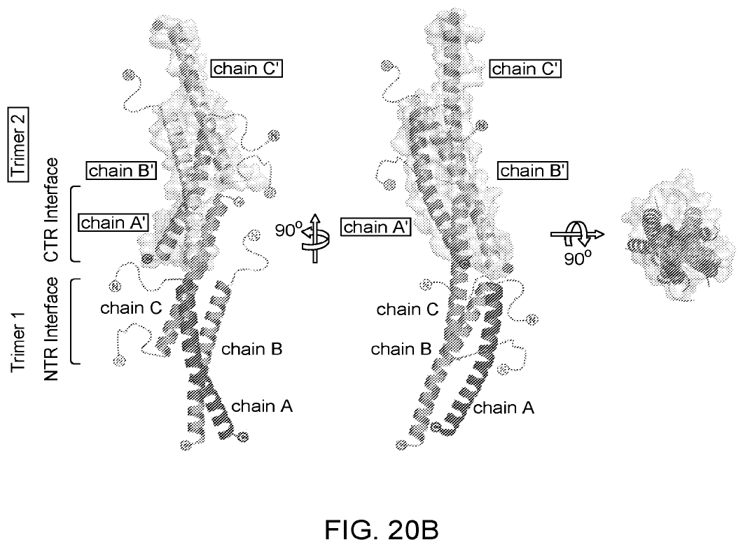

Monkeypox A29L was found to bear high sequence similarlity to vaccinia A27L. In particular MPX A29L derived from GenBank AAN78220.1 and A27L derived from AAA60882.1 are 94.5% identical on the amino acid level. The structure of SPX A27L from Chang et al. 2013. PLoSPathogens is shown in FIGS. 20A and 20B. During virus entry, A27 mediates the attachment of mature vaccinia virus to cell surface heparan sulfate. A27 also tethers a viral fusion suppressor protein, A26, to mature virions. During virion morphogenesis, A27 mediates mature virus transport in infected cells. The protein adopts a unique antiparallel dimer-of-trimers conformation, in which one interface (the NTR) mediates coiled-coil trimer formation, and another interface (the CTR) mediates dimer formation of the trimers. Abrogation of this structural assembly (for example, through targeted mutagenesis of the NTR and CTR) severely impacts viral entry—furthermore, some disulfide bonds are important for maintaining the integrity of the complex and its interaction with other viral components (for example A26 in SPX). There are no predicted TM regions for this antigen.

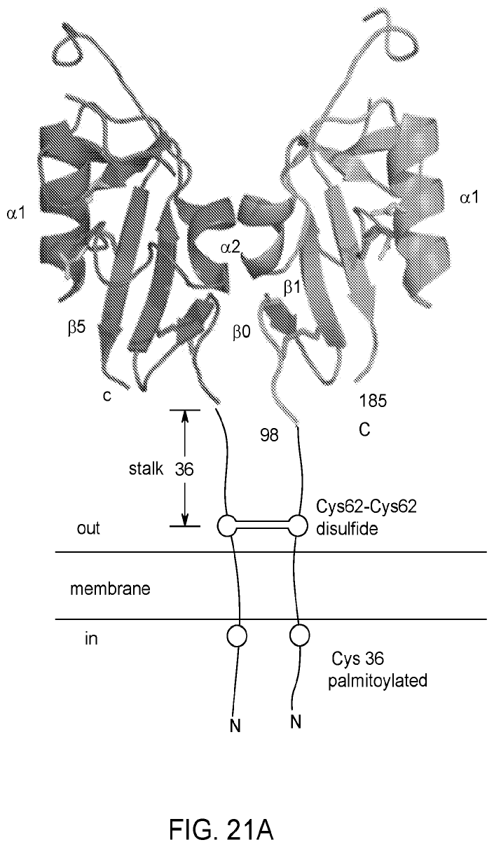

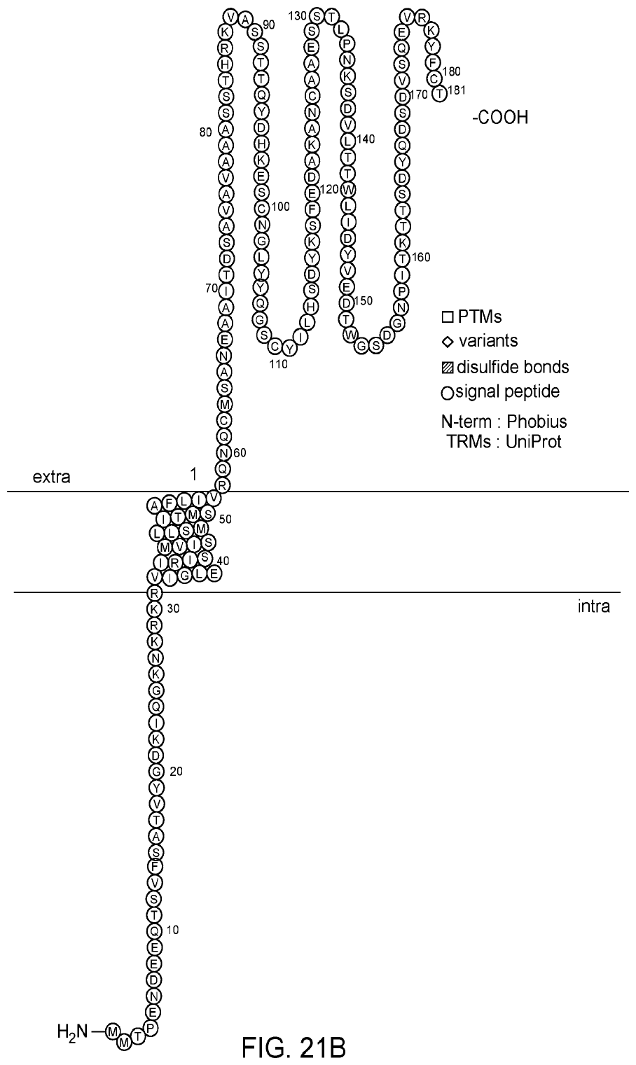

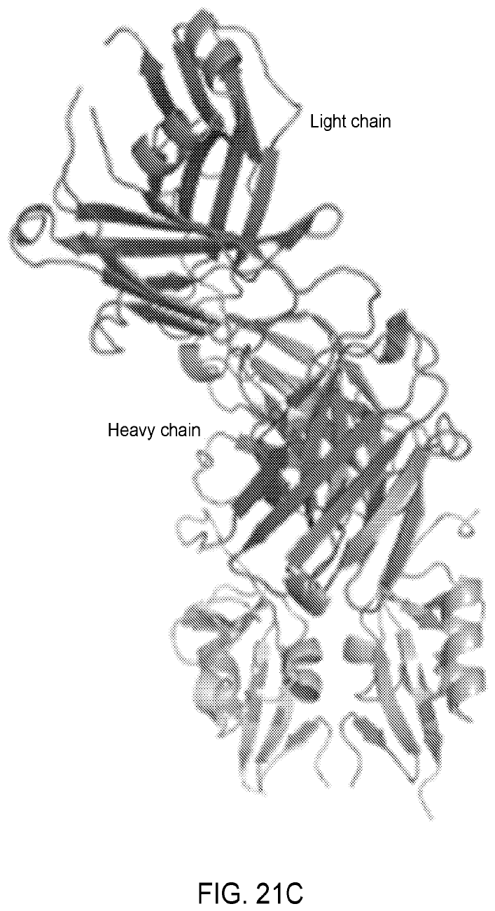

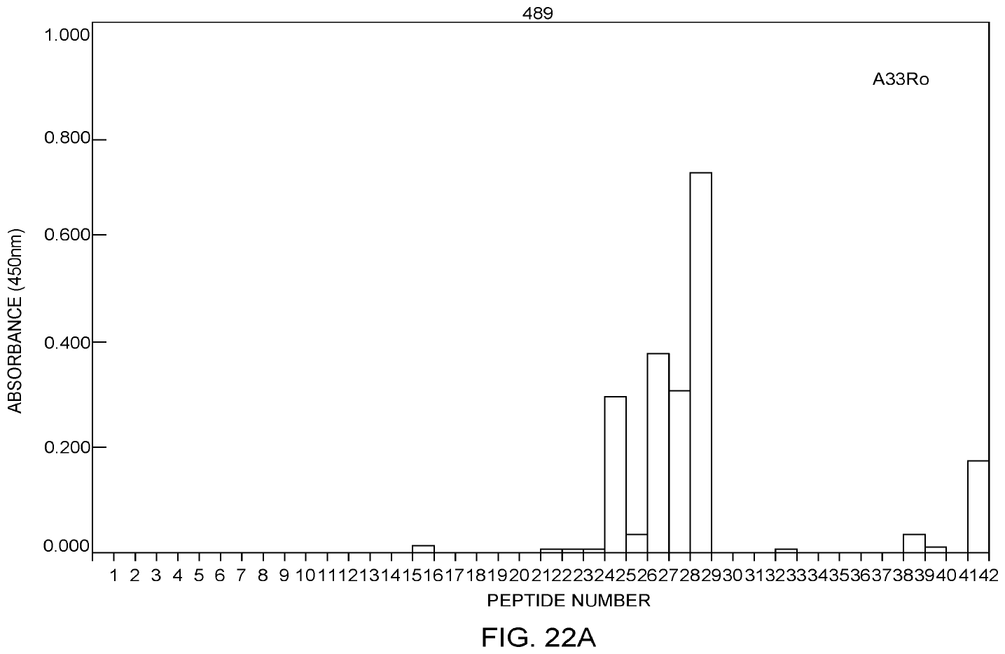

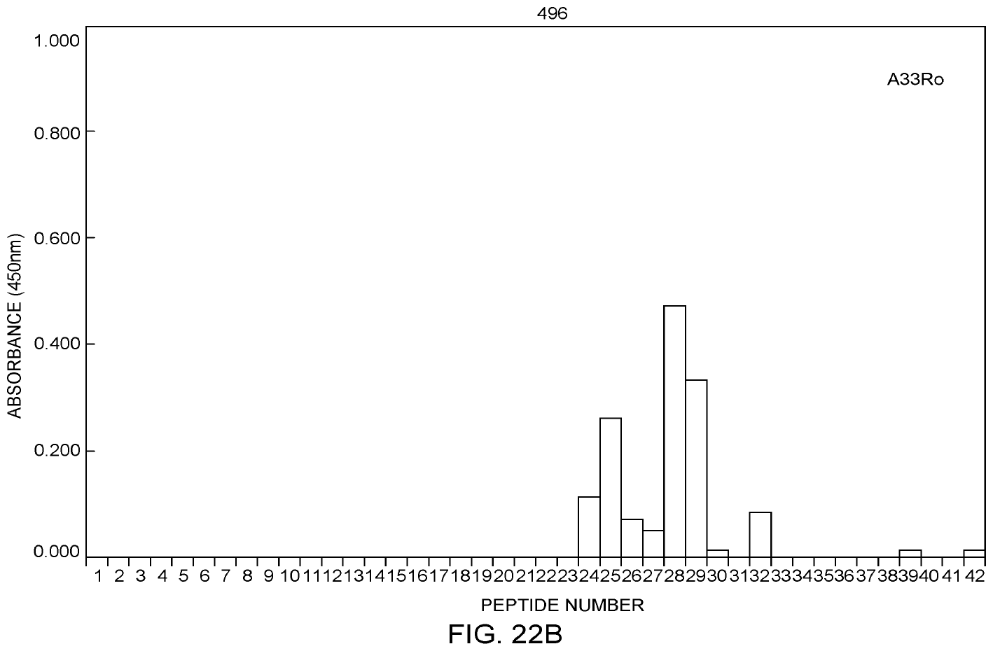

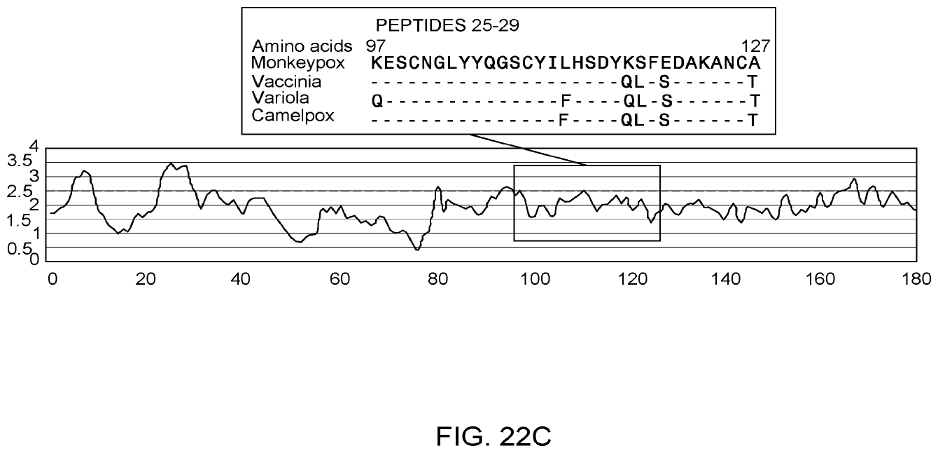

Monkeypox A35R was found to bear high sequence similarity to vaccinia A33R. In particular sp|P68617|A33_VACCW Protein A33 OS=Vaccinia virus (strain Western Reserve) OX=10254 GN=VACWR156 PE=1 SV=1 and tr|Q8V4U4|Q8V4U4_MONPZ A35R OS=Monkeypox virus (strain Zaire-96-I-16) OX=619591 GN=A35R PE=4 SV=1 are 93.5% identical on the amino acid level. The structure of poxvirus A33 from J. Virol. 2010, https://www.ncbi.nlm.nih.gov/pmc/articles/PMC2820914/reveals a dimer of unique C-Type Lectin-Like domains, and is shown in FIGS. 21A and 21B. The structure revealed C-type lectin-like domains (CTLDs) that occur as dimers. Structural comparisons show that the A33 monomer is a close match to the Link module class of CTLDs but that the A33 dimer is most similar to the natural killer (NK)-cell receptor class of CTLDs whose ligands include MHC-related proteins. Structural data on Link modules and NK-cell receptor-ligand complexes suggest a surface of A33 that could interact with viral or host ligands. The dimer interface is well conserved in all known A33 sequences, indicating an important role for the A33 dimer. The location of the putative ligand binding site is marked (*). The location of the surface implicated in the MAb 1G10 epitope and contributed by residues 118 and 120 is indicated on the “b” monomer (blue). B cell epitope mapping from Heraud et al. (https://doi.org/i0.4049/jimmunol.177.4.2552) by Elisa using linear epitipes is shown in FIG. 22A-22C.

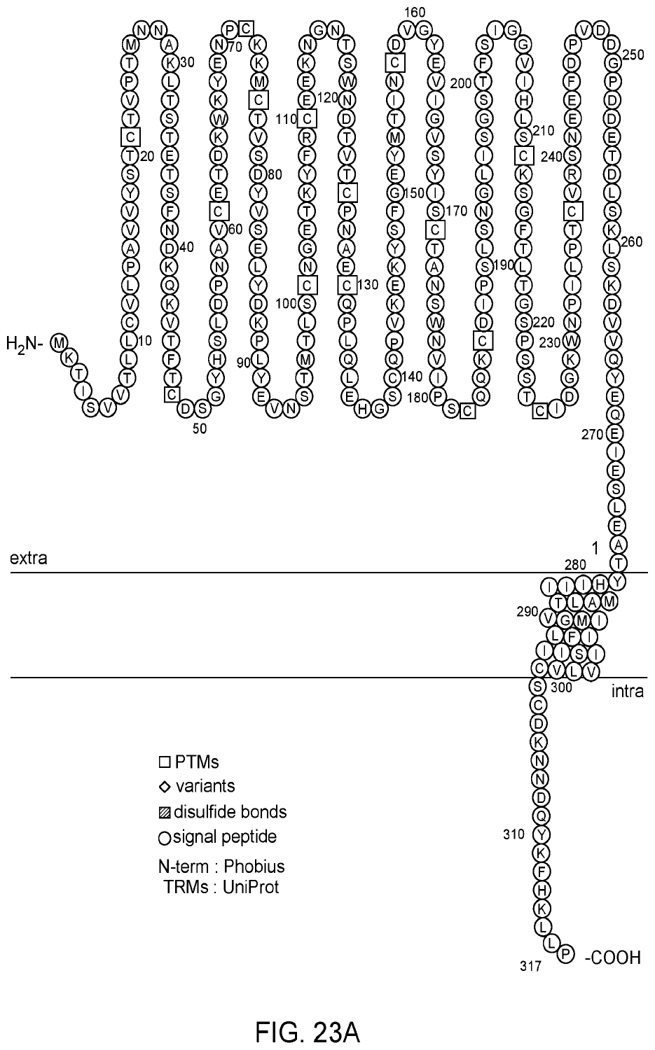

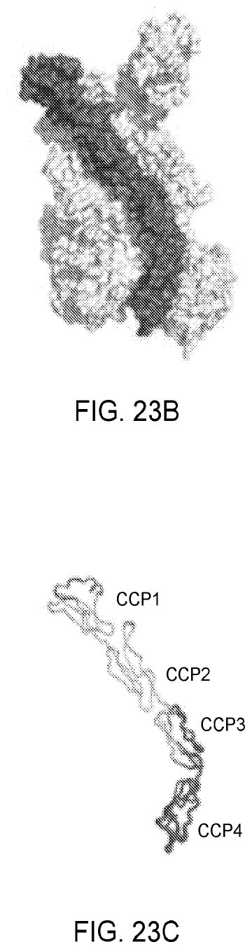

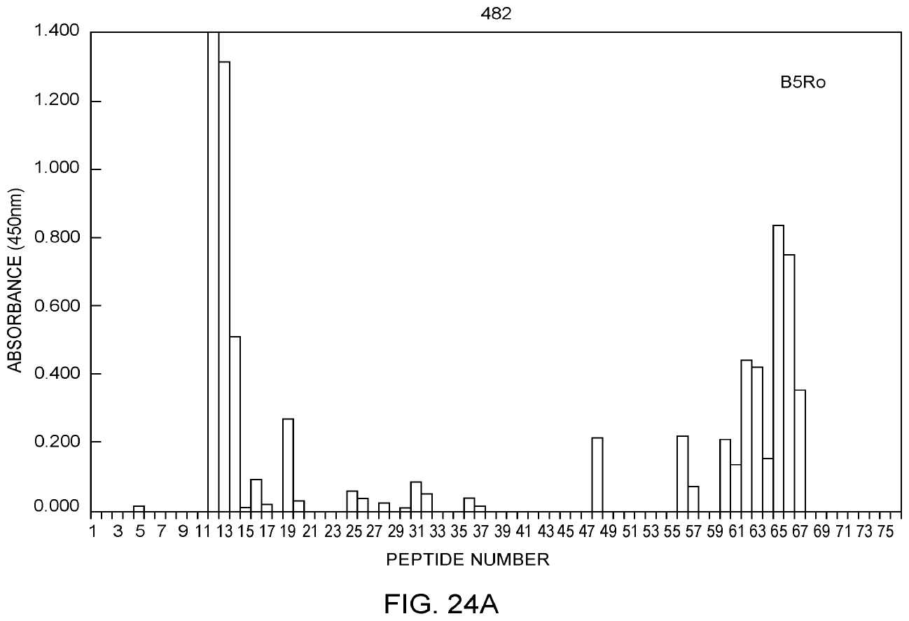

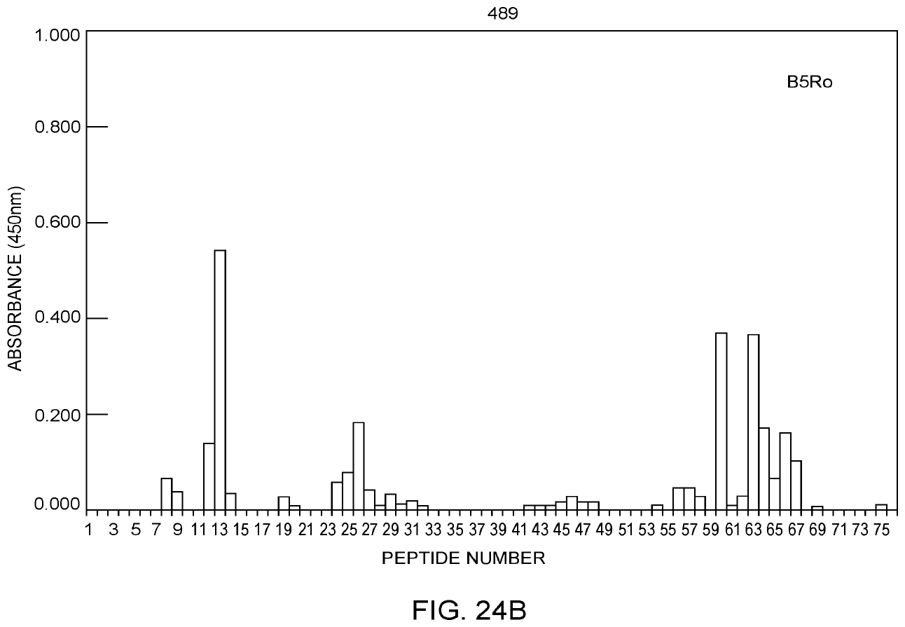

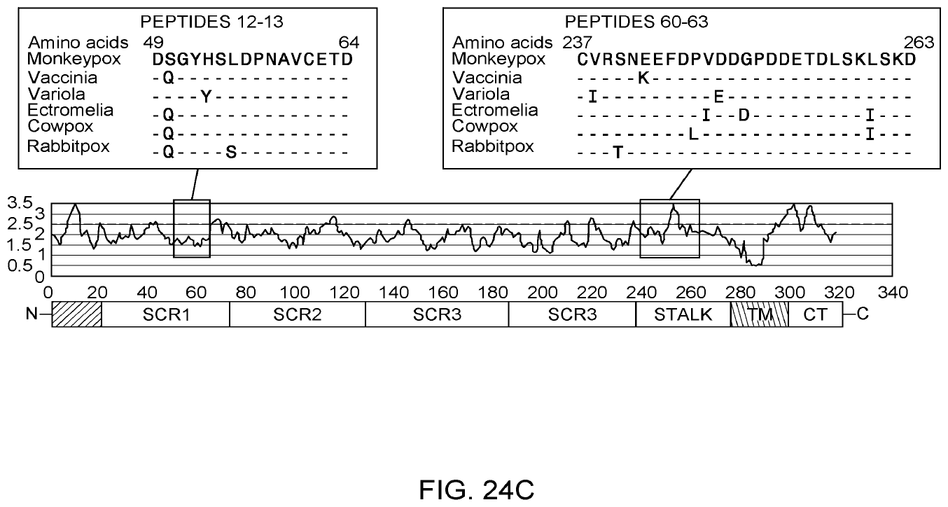

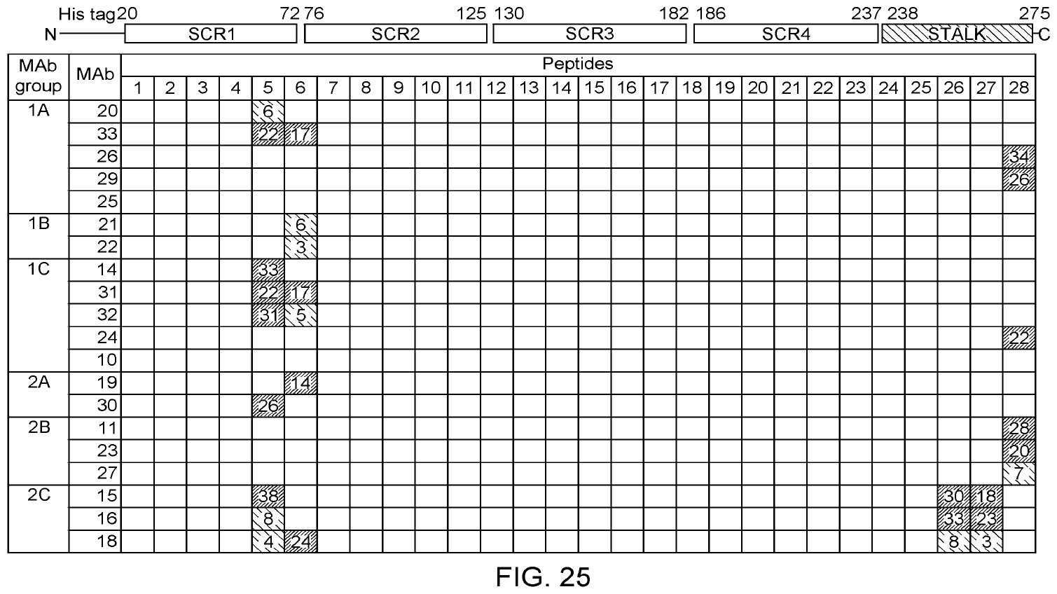

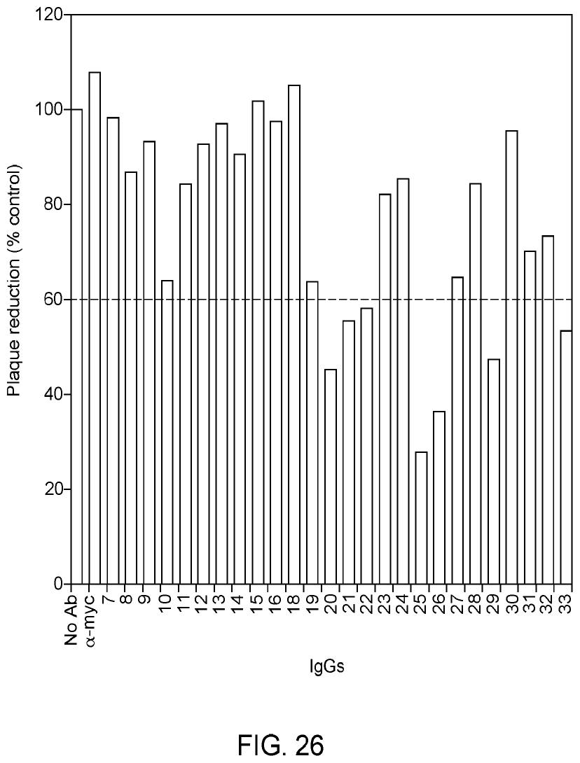

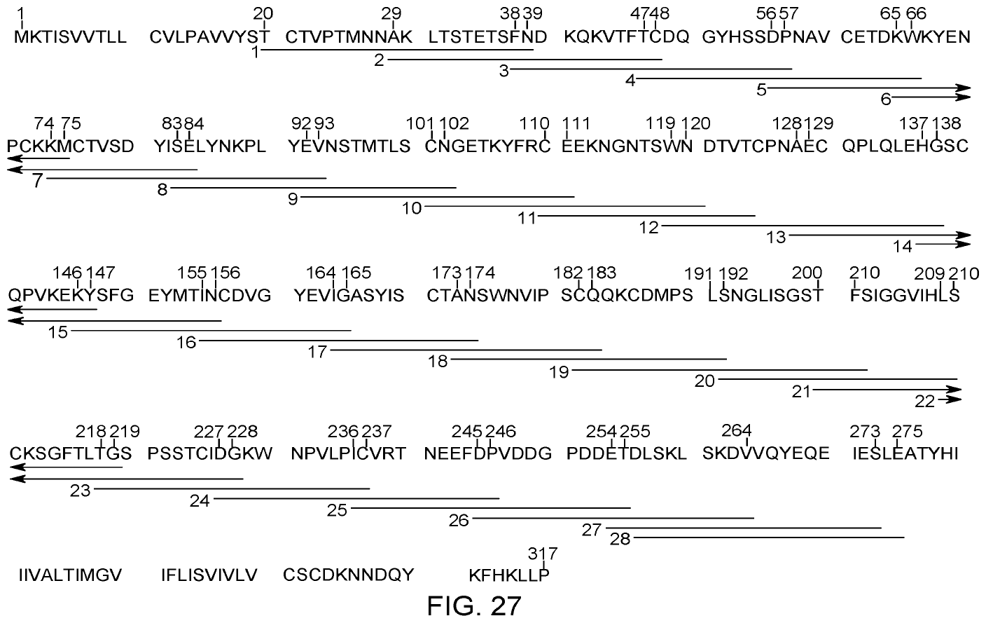

Monkeypox B6R was found to bear high sequence similarlity to vaccinia B5R. In particular tr|Q8V4S2|Q8V4S2_MONPZ B6R OS=Monkeypox virus (strain Zaire-96-I-16) OX=619591 GN=B6R PE=3 SV=1 and sp|Q01227|B5_VACCW Protein B5 OS=Vaccinia virus (strain Western Reserve) OX=10254 GN=PS/HR PE=1 SV=1 are 94.5% identical on the amino acid level. The structure of MPX B6R from EMBO J. 2016 10.15252/embj.201593673 is shown in FIG. 23A-23C. MPX B6R is homologous to smallpox virus homolog (SPICE) which binds and inhibits complement C₃b. B cell epitope mapping from Heraud et al. (https://doi.org/10.4049/jimmunol.177.4.2552) by Elisa using linear epitipes is shown in FIG. 24A-24C. It was observed by Aldaz-Carroll et al Journal of Virology 2005 (https://doi.org/10.1128/JVI.79.10.6260-6271.2005) that a wide set of monoclonal antibodies bind the same linear eptiopes on vaccinia B5R (see FIG. 25, FIG. 26, and FIG. 27).

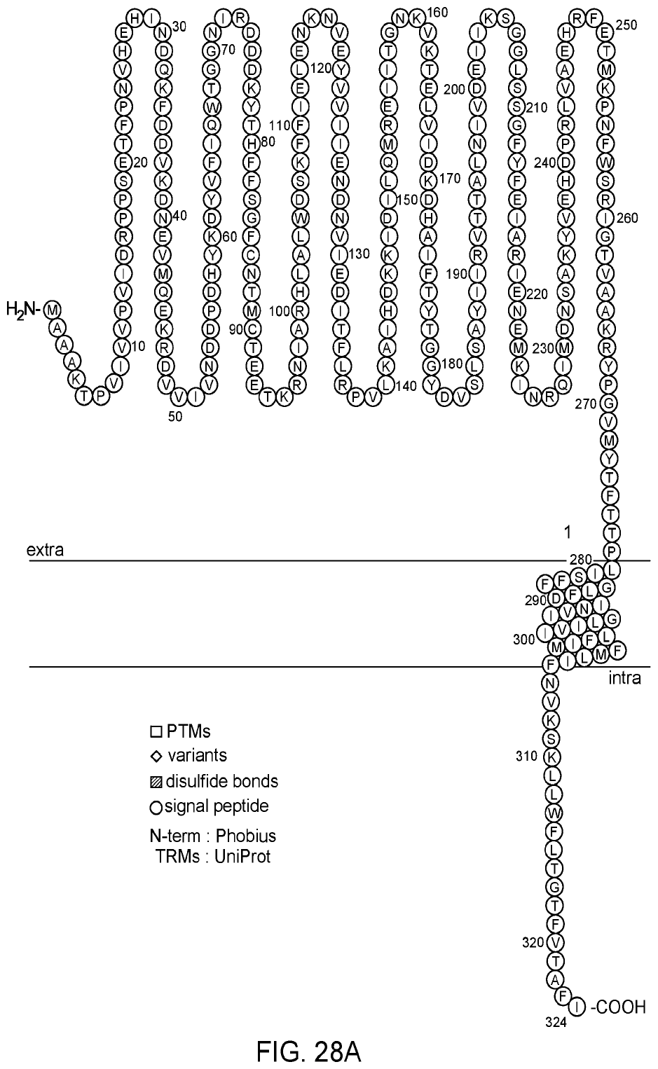

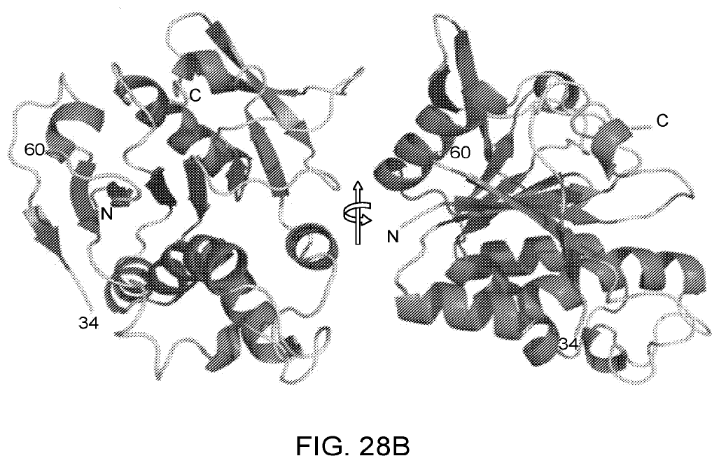

Monkeypox H3L was found to bear high sequence similarlity to vaccinia H3L. In particular tr|Q3I8S1|Q3I8S1_MONPV H3L OS=Monkeypox virus OX=10244 GN=H3L PE=4 SV=1 and sp|P07240|H3_VACCW Envelope protein H3 OS=Vaccinia virus (strain Western Reserve) OX=10254 GN=VACWR101 PE=1 SV=1 are 93.5% identical on the amino acid level. The structure of H3L from Singh et al., J Virol (2016) is shown in FIG. 28A-28B.

Epitopes have been predicted along monkeypox T cell antigen sequences in literature (downloaded from the Immune Epitope Database and Analysis Resource IEDB https://www.iedb.org/), and separately by the present inventors. In addition to the Epitoipe Database plot from the literature, the inventors generated three plots showing (i) how many alleles are covered by predicted epitopes for each AA residue (the “allele” plot), (ii) how many unique epitope sequences are covering each AA residue (the “sequence” plot), and (iii) how many unique pairings of epitope and alleles are there (the “sequence_allele” plot), meaning that if an epitope X is predicted to bind both allele Y and allele Z, it will be counted twice.

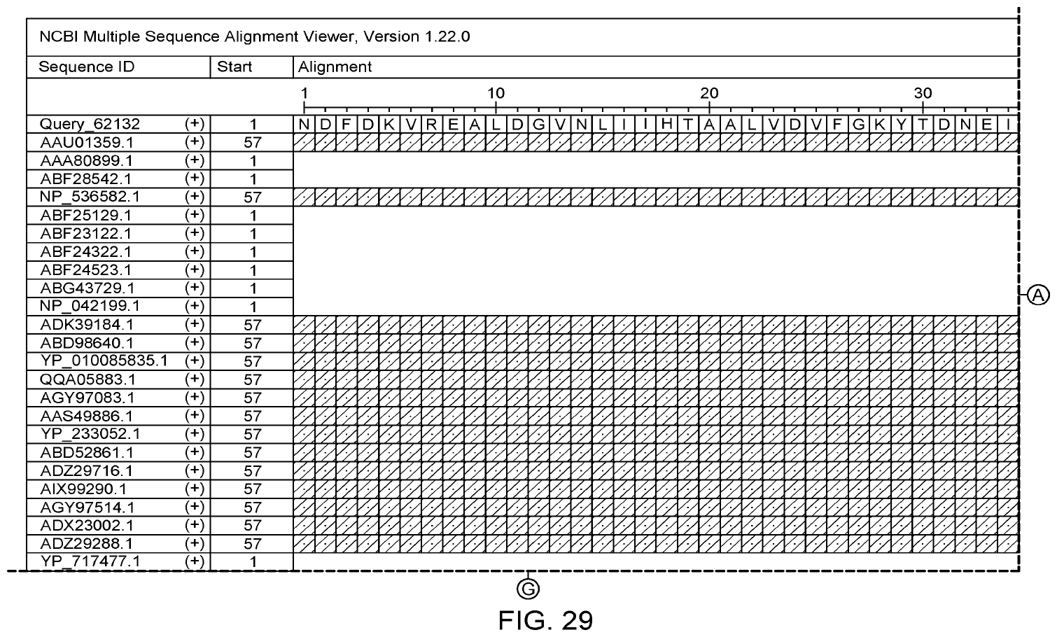

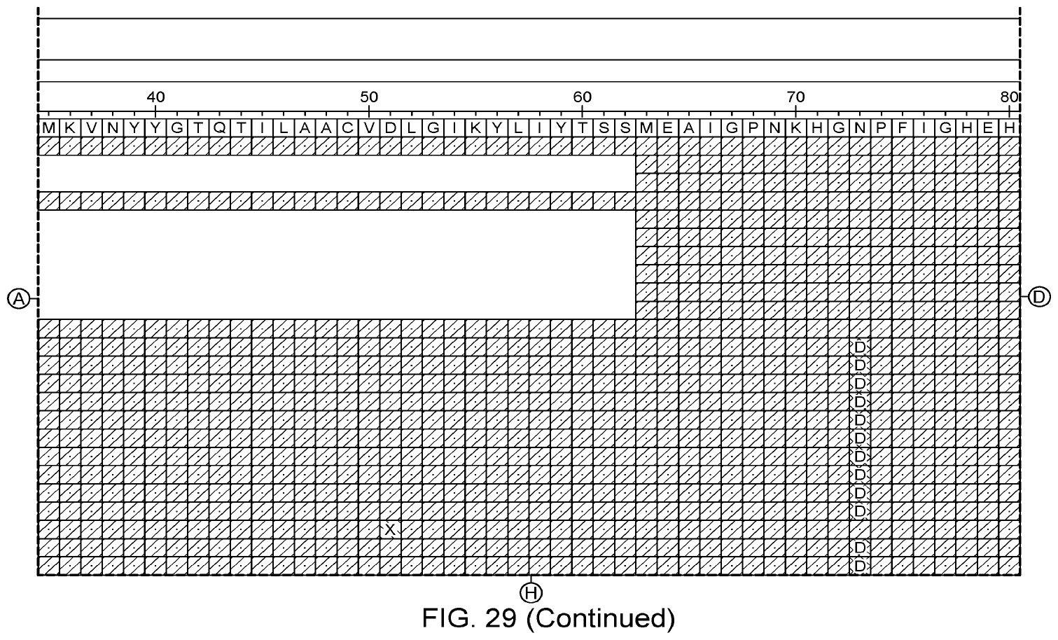

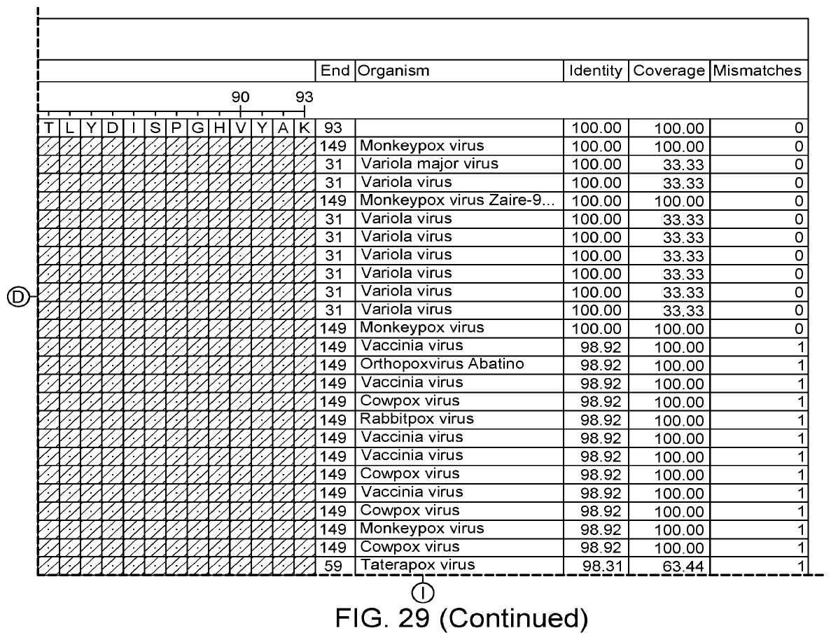

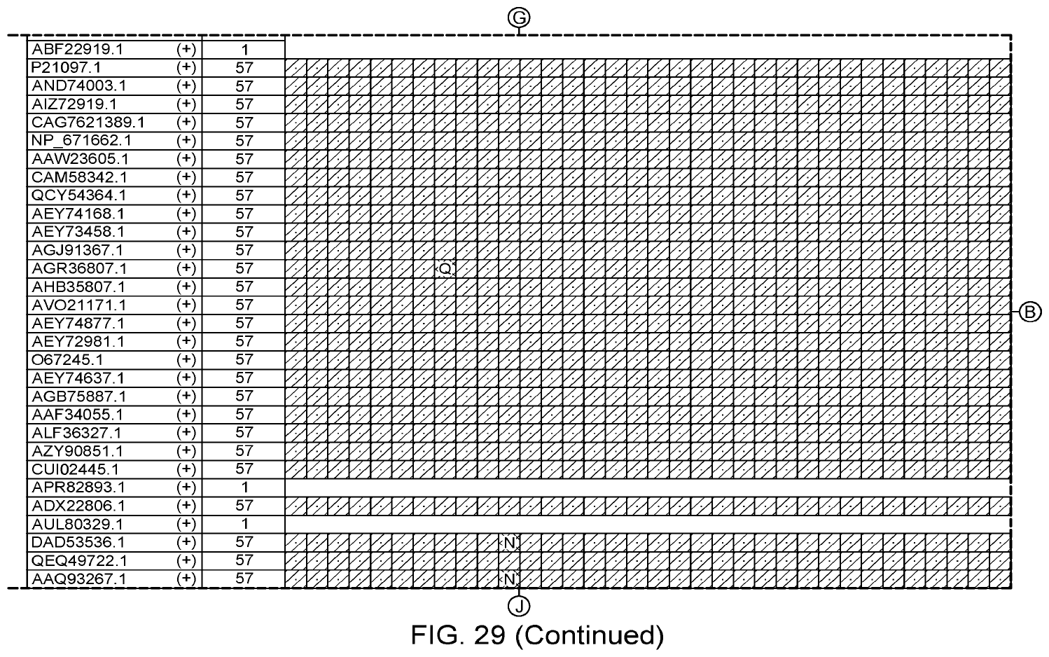

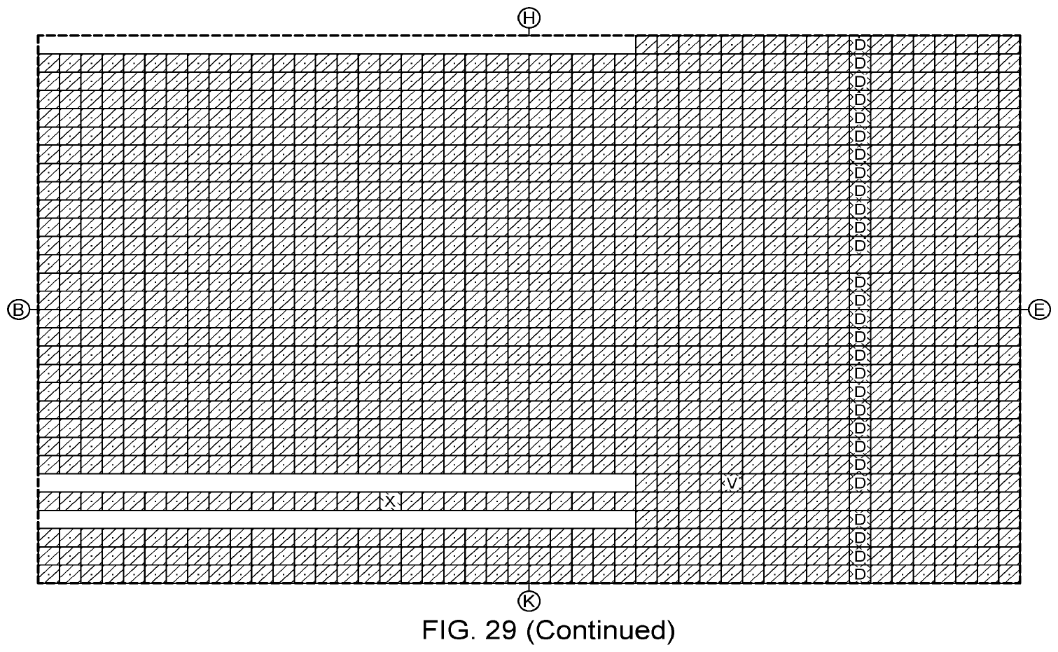

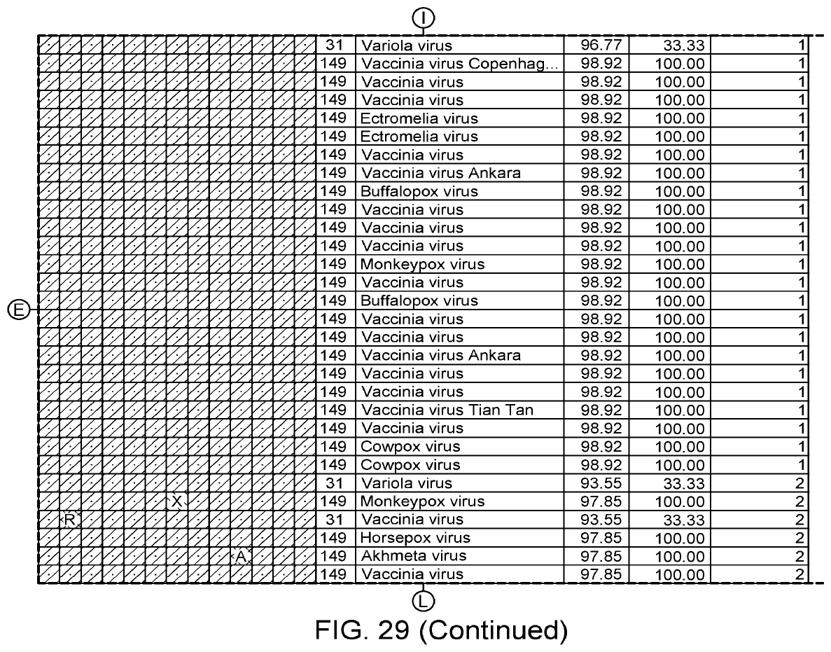

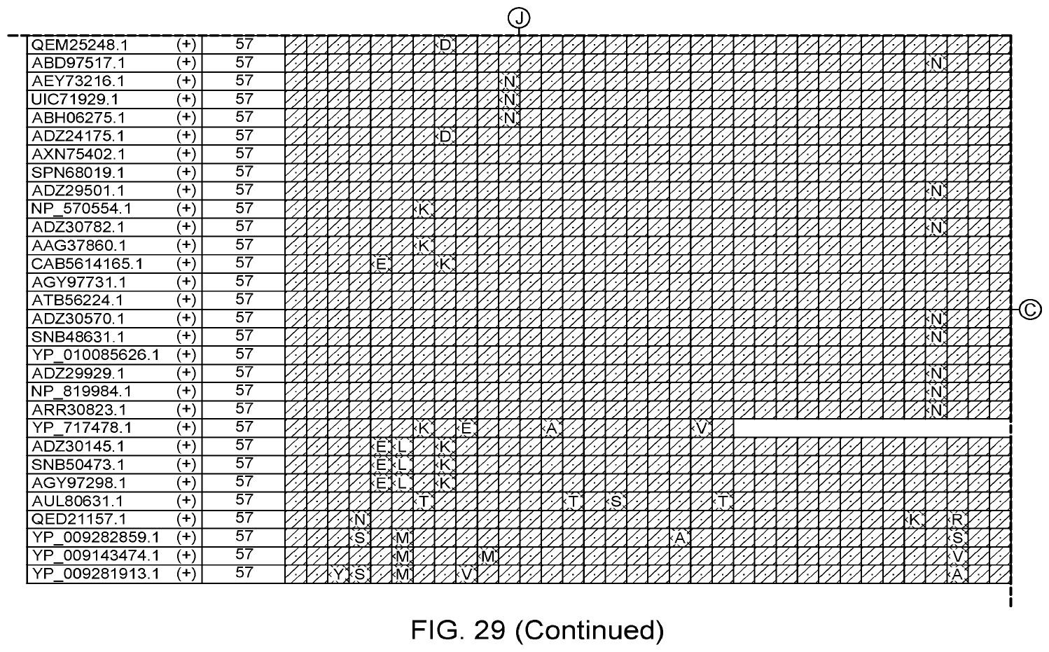

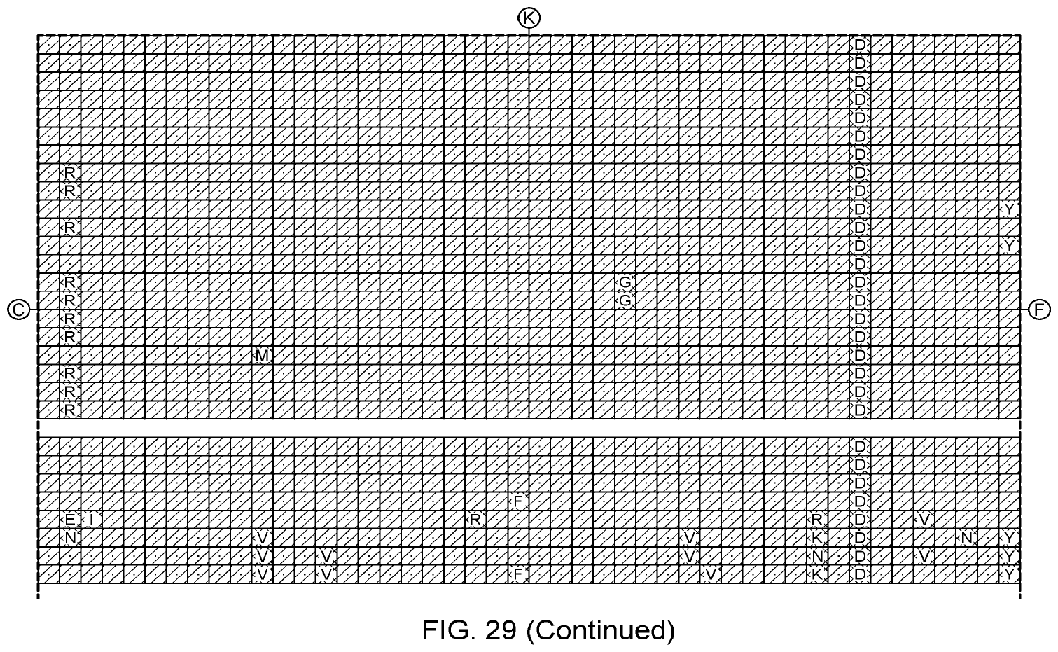

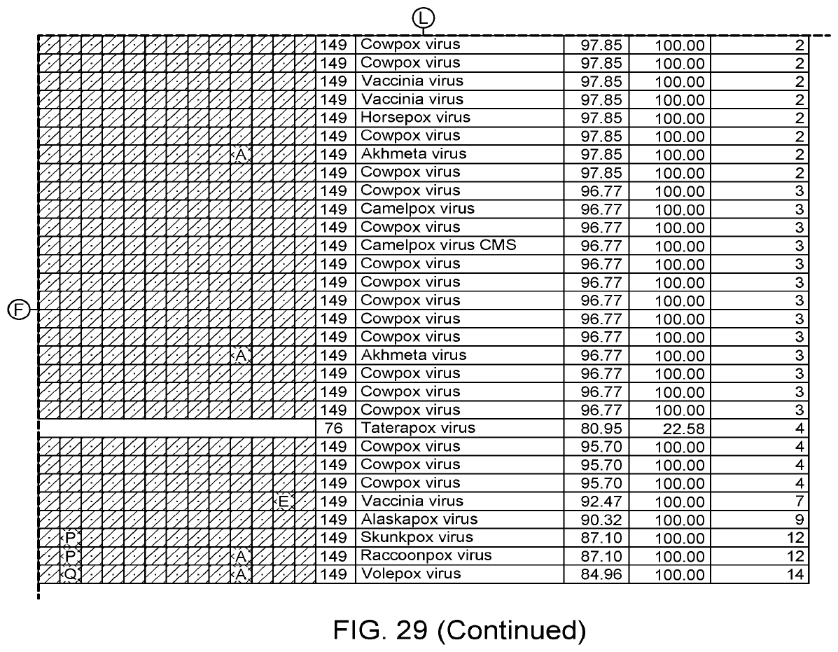

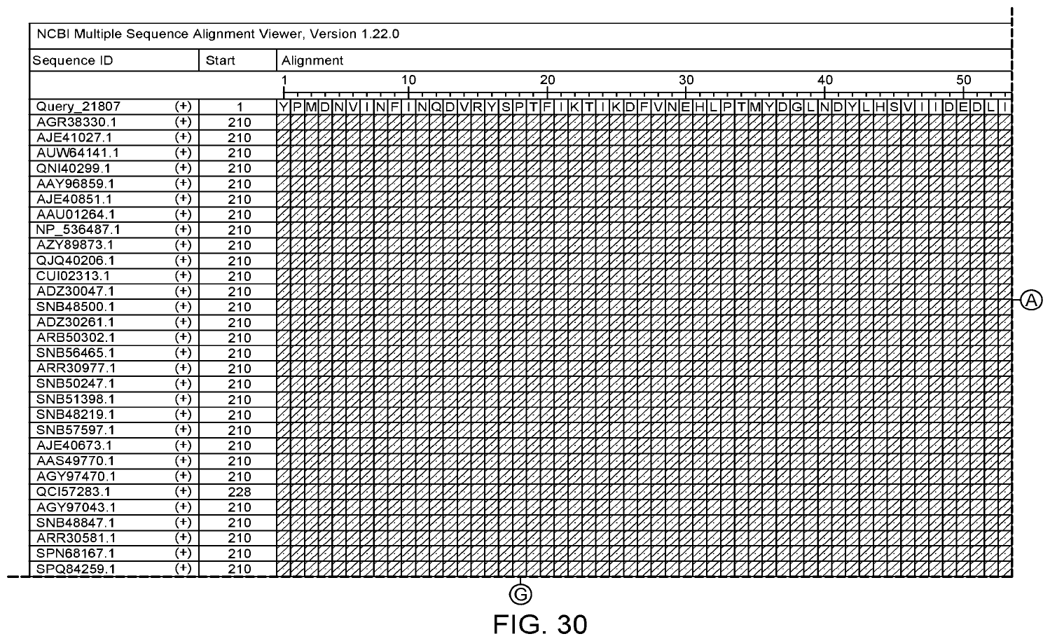

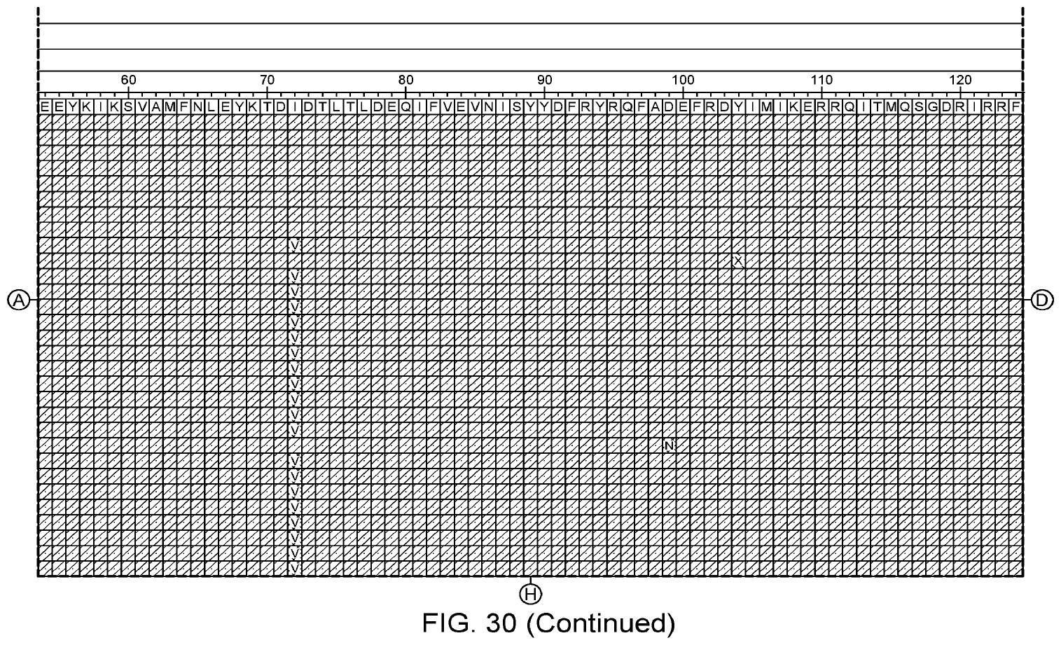

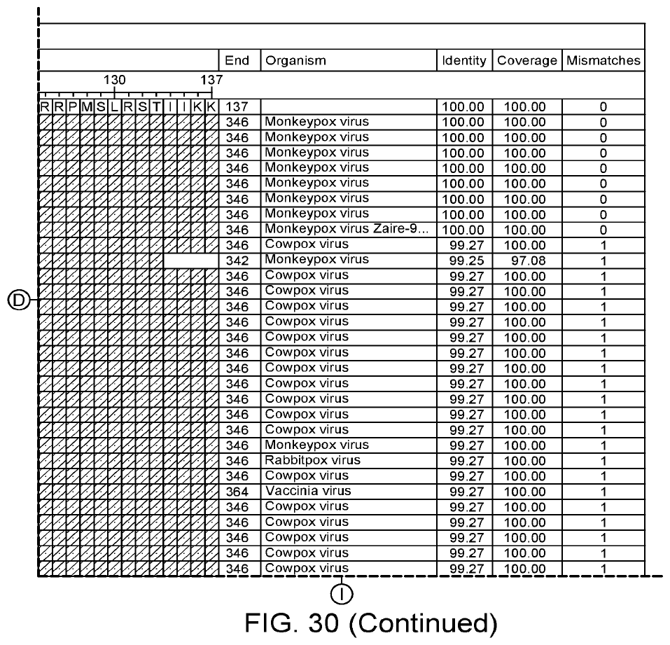

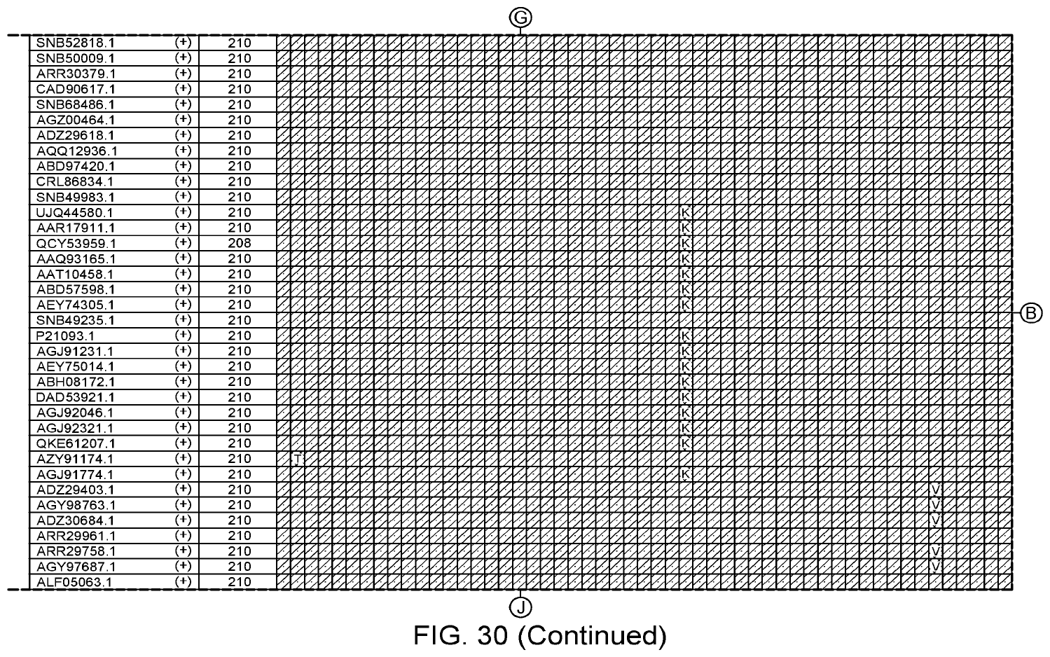

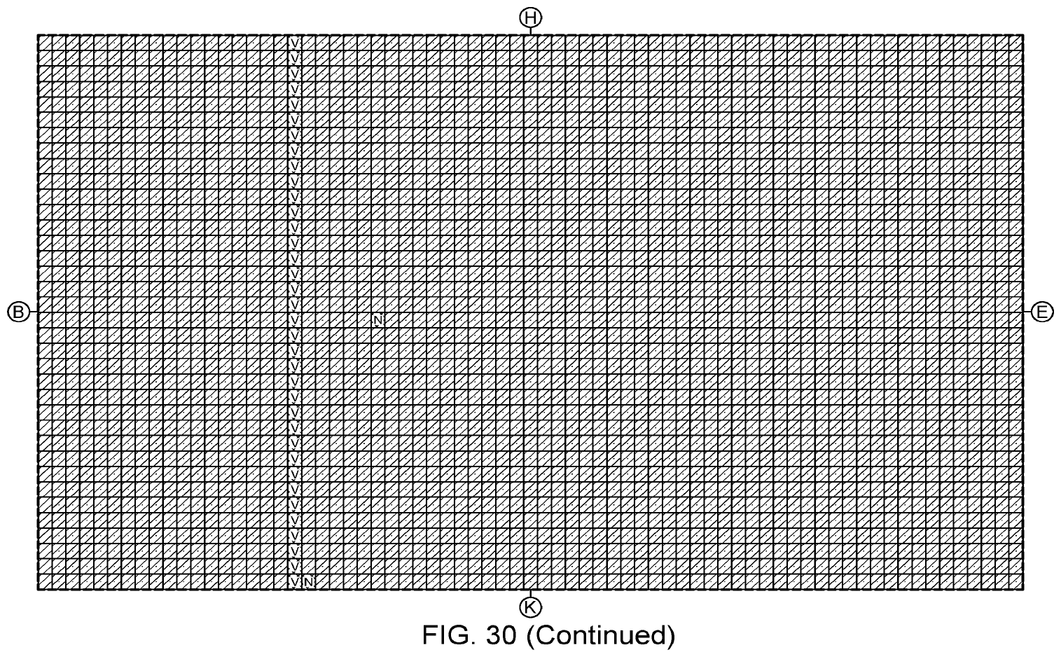

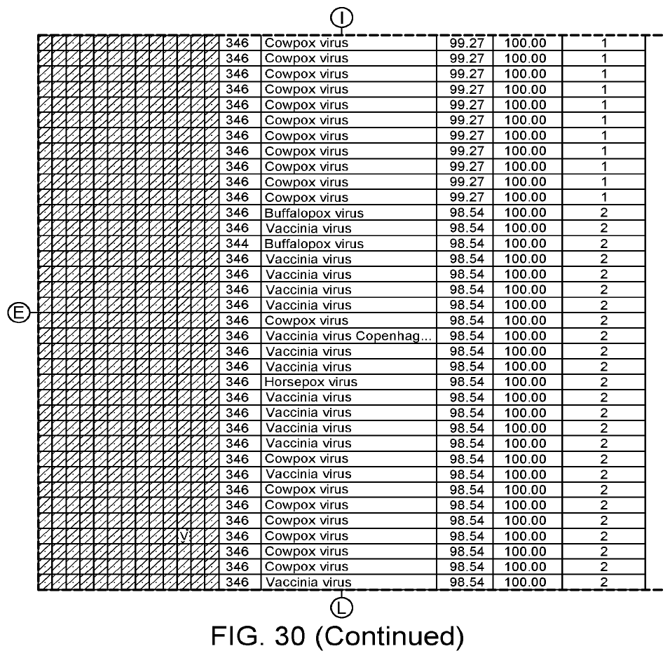

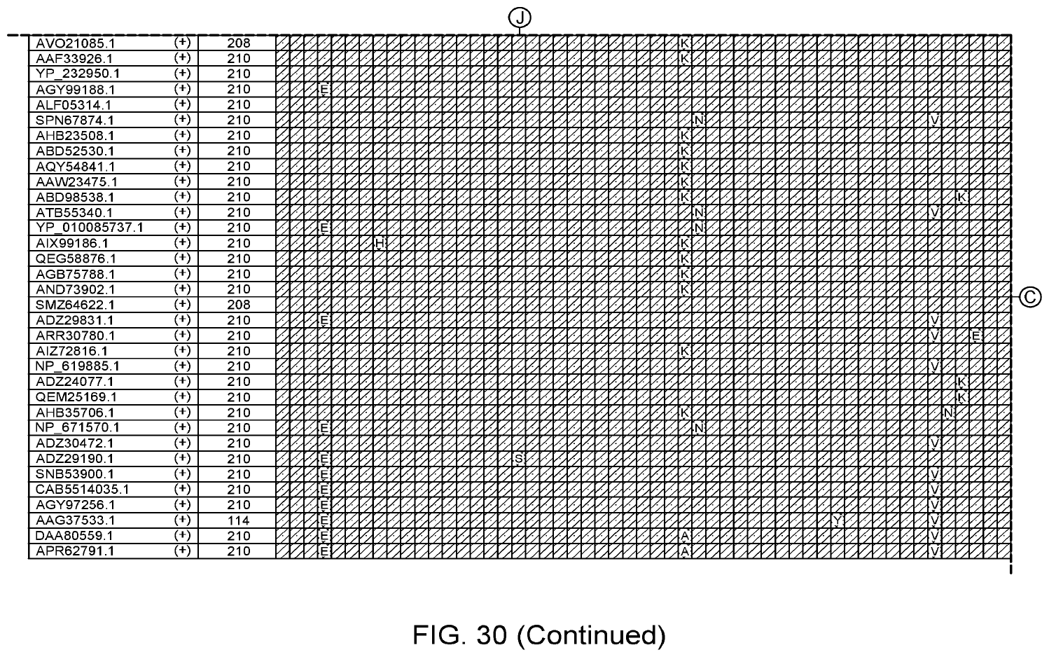

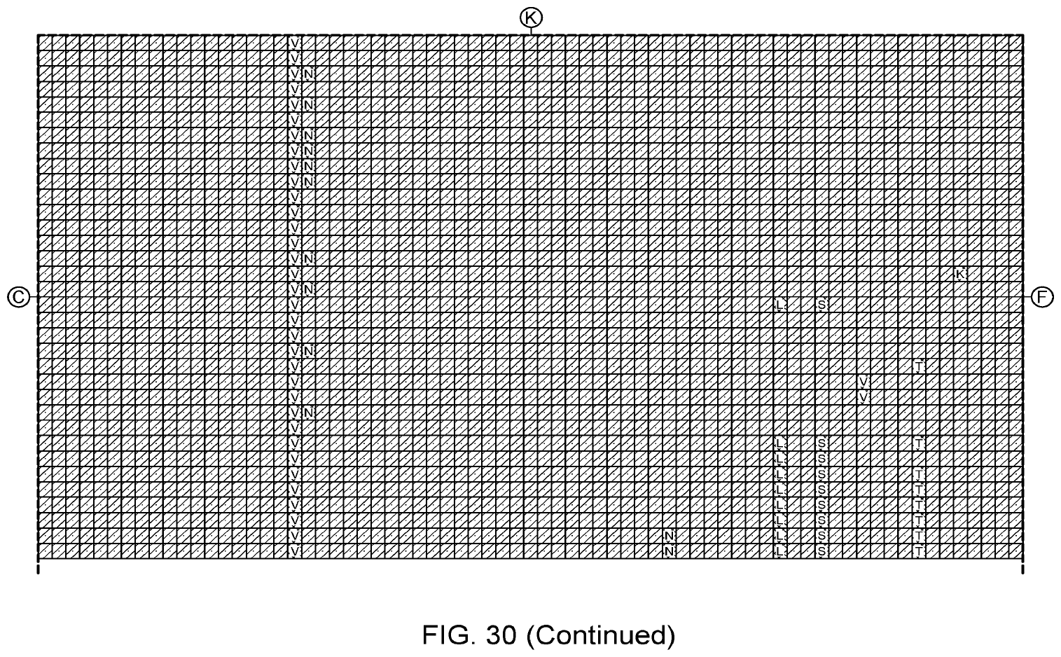

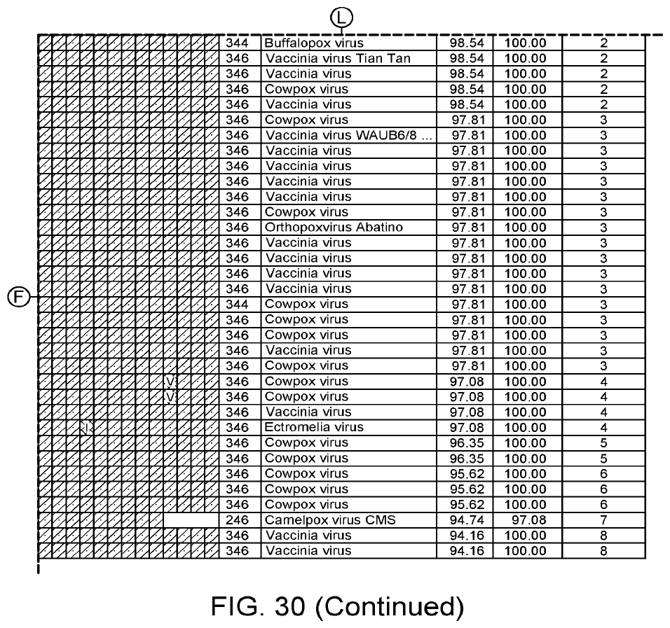

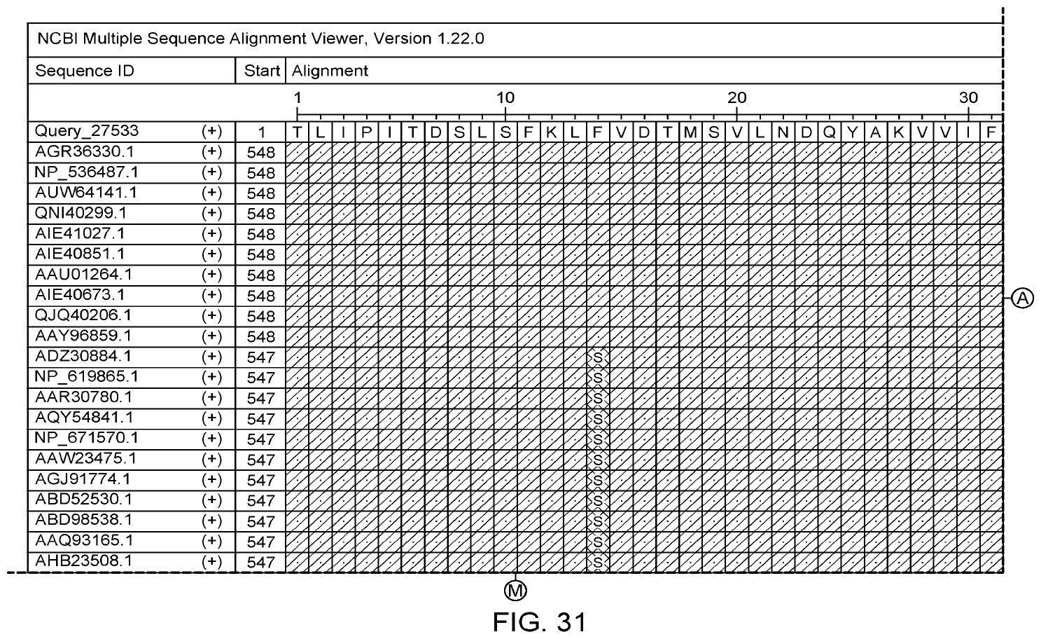

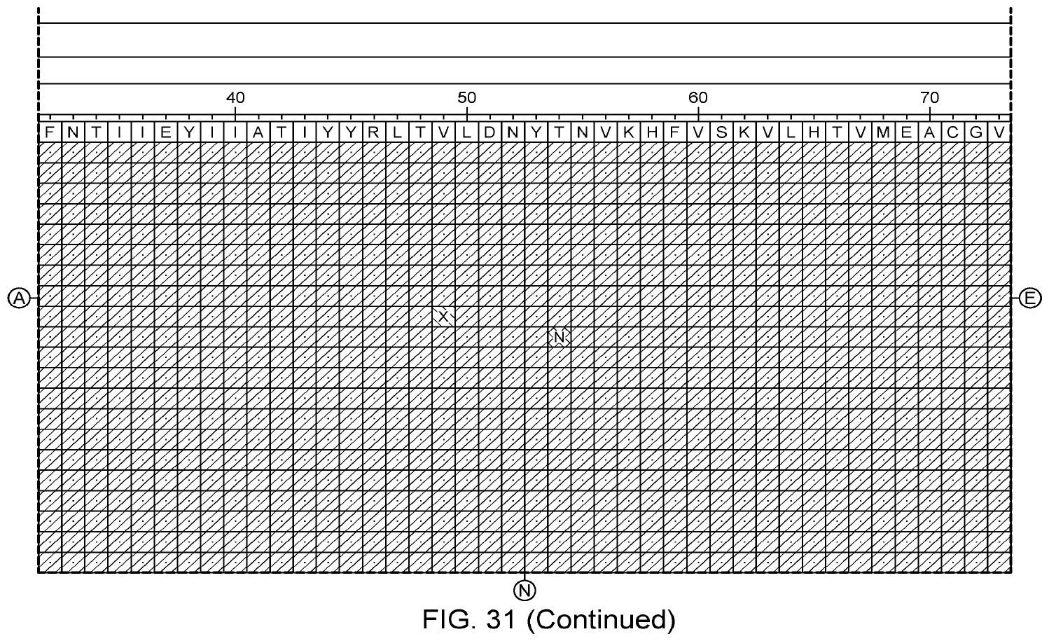

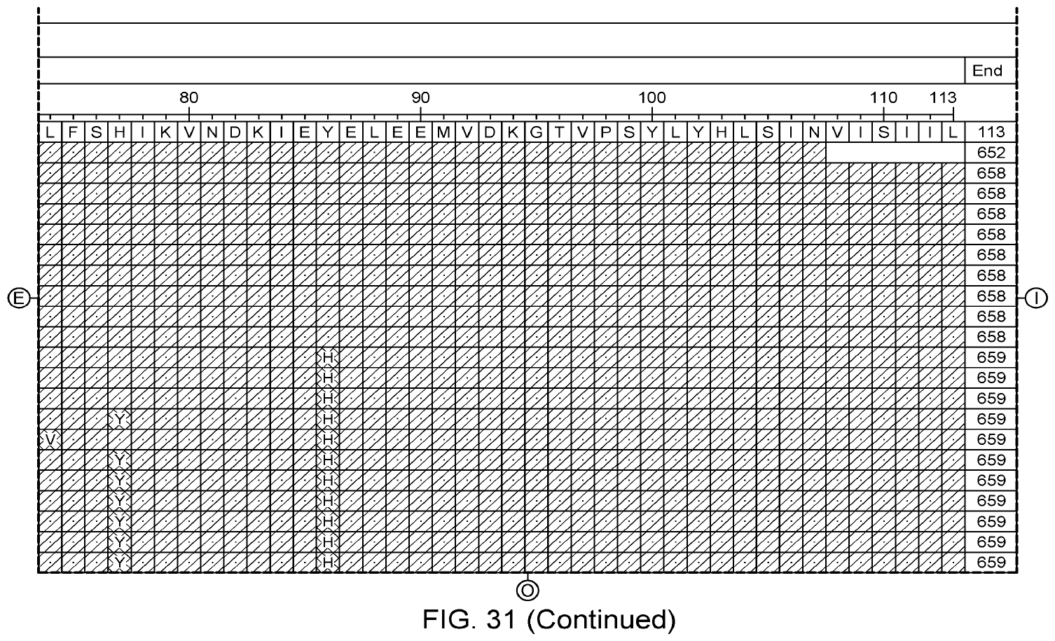

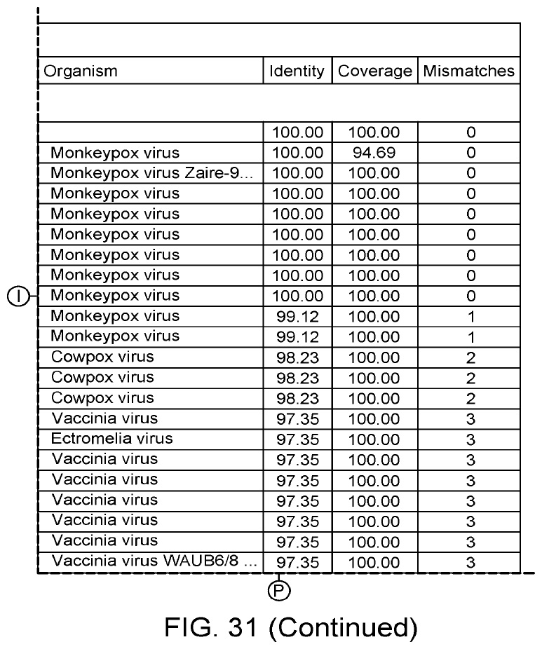

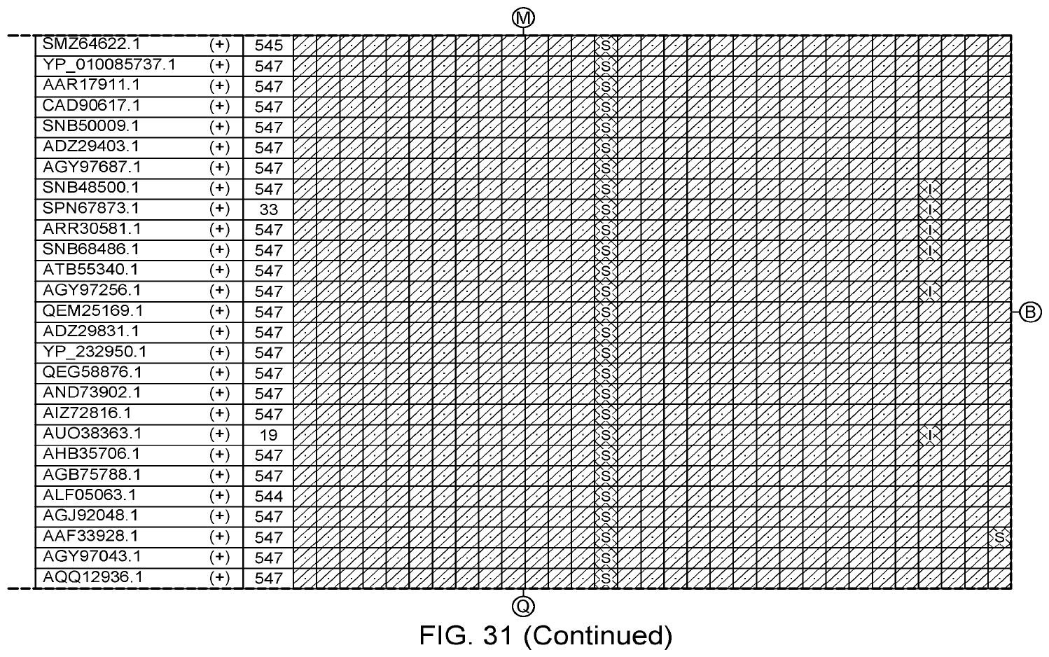

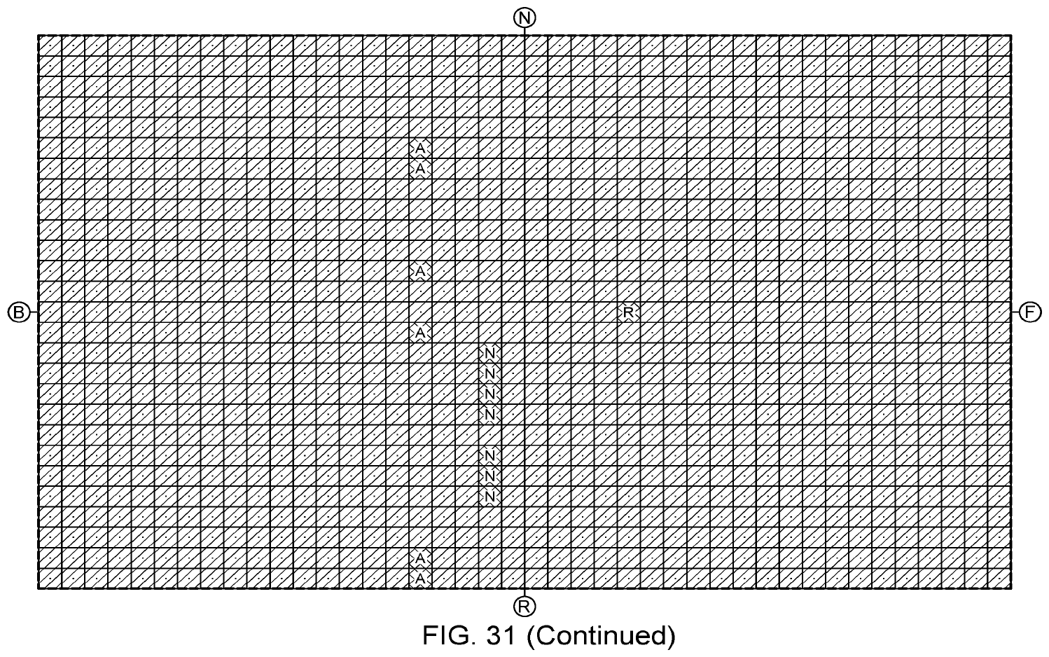

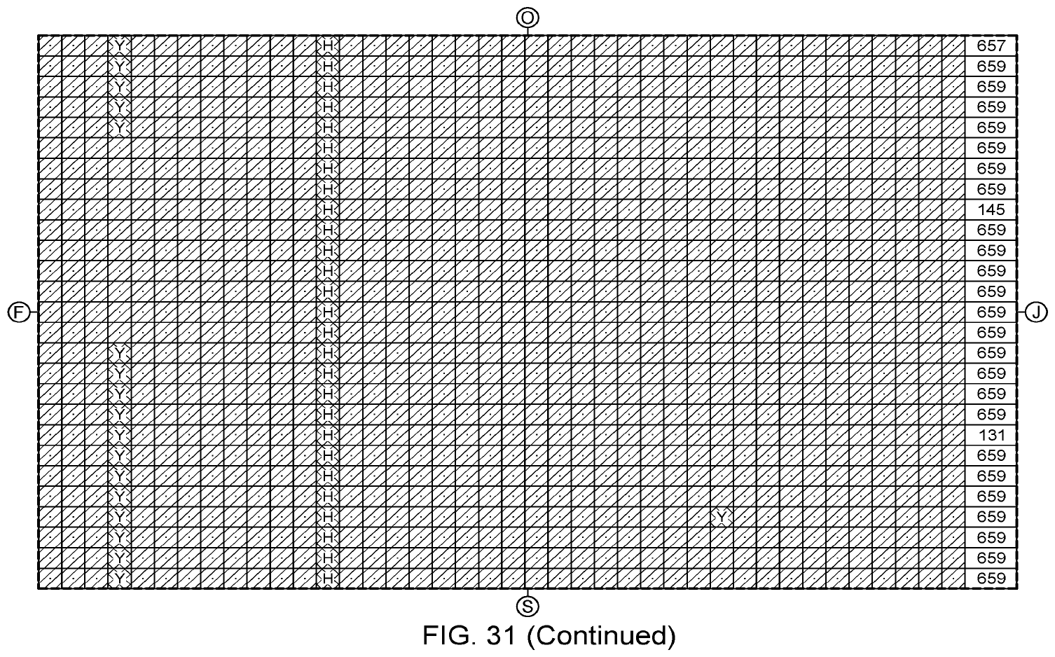

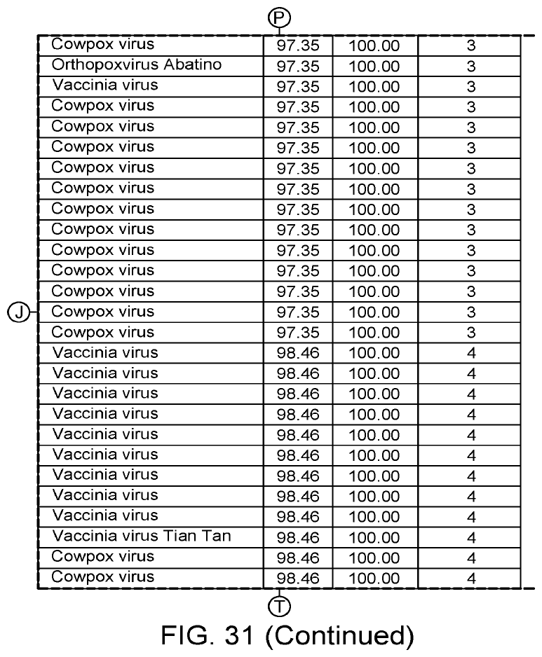

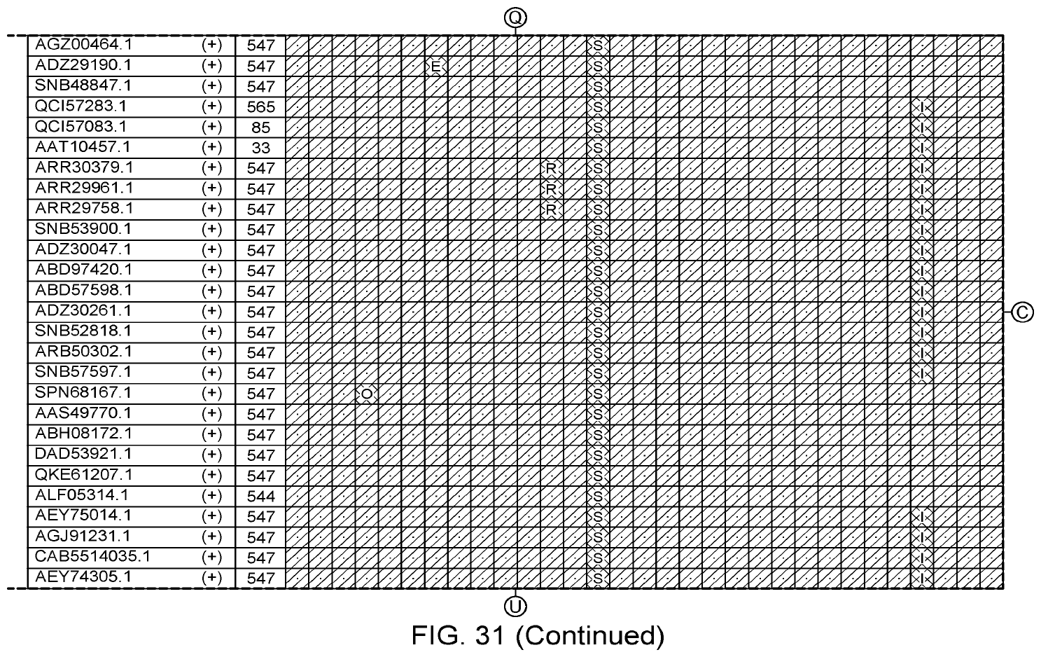

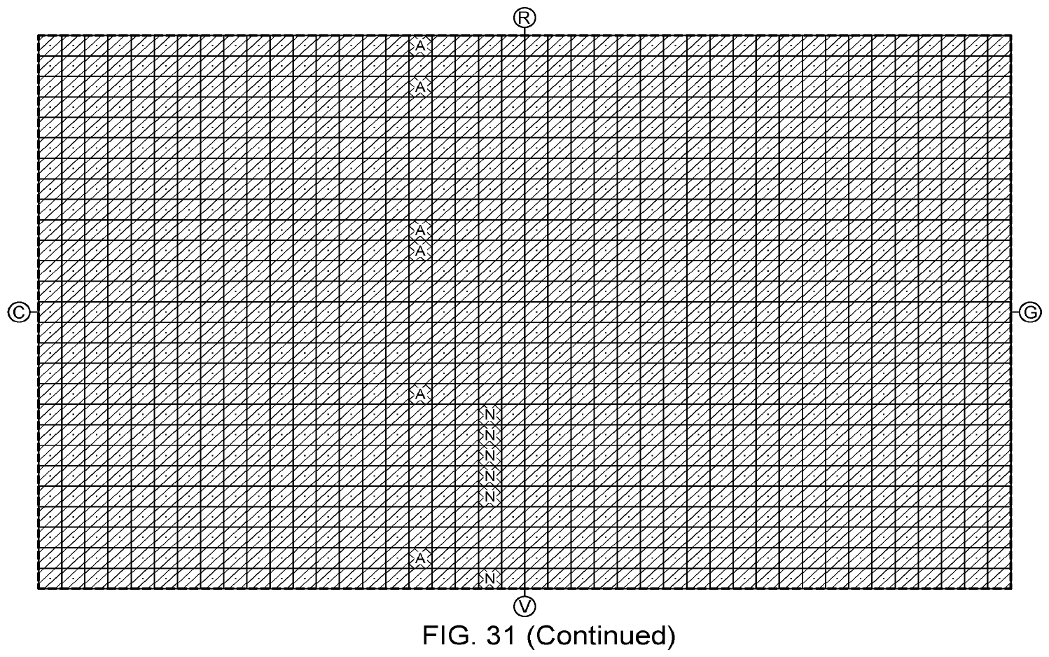

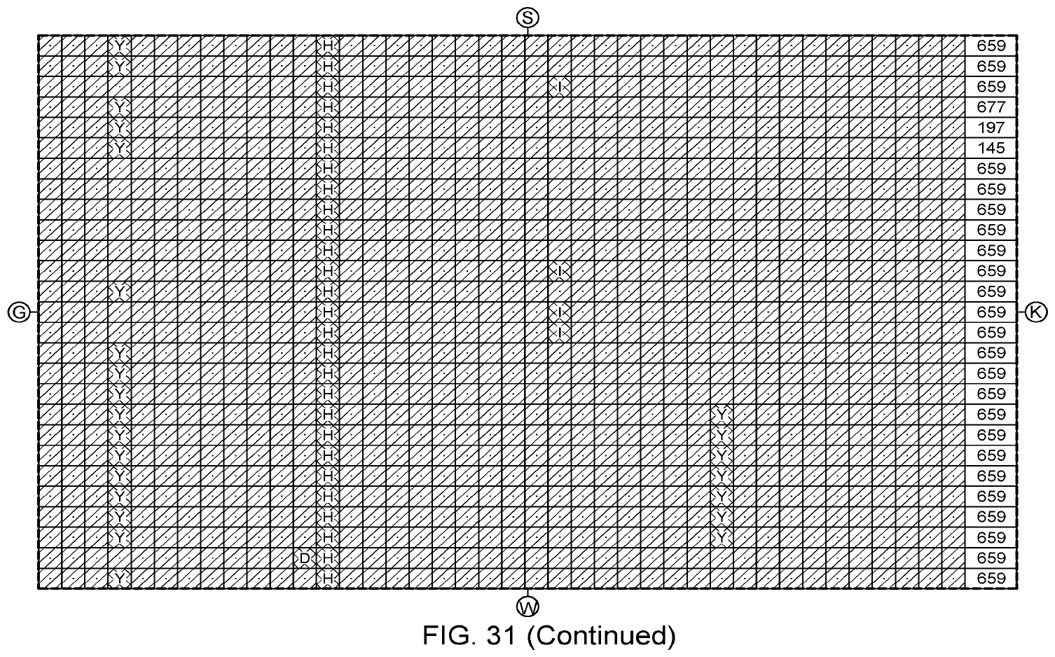

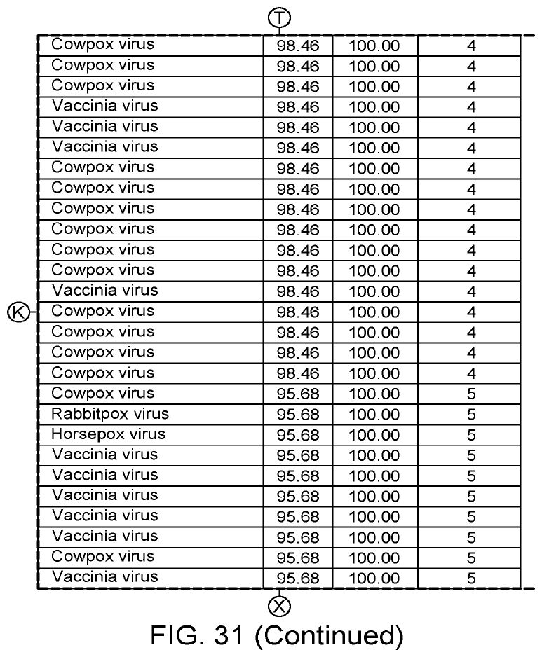

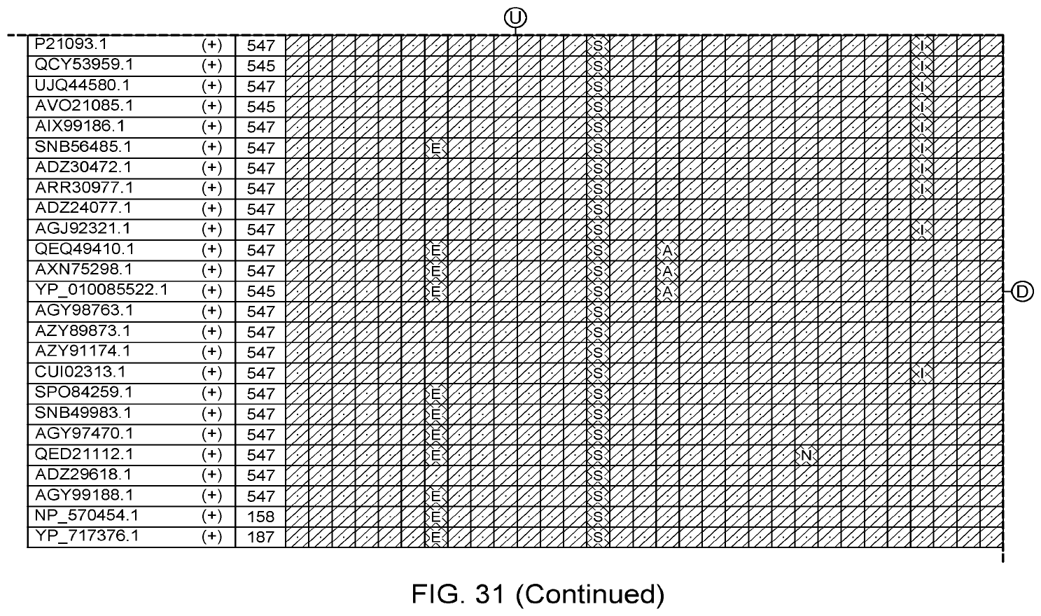

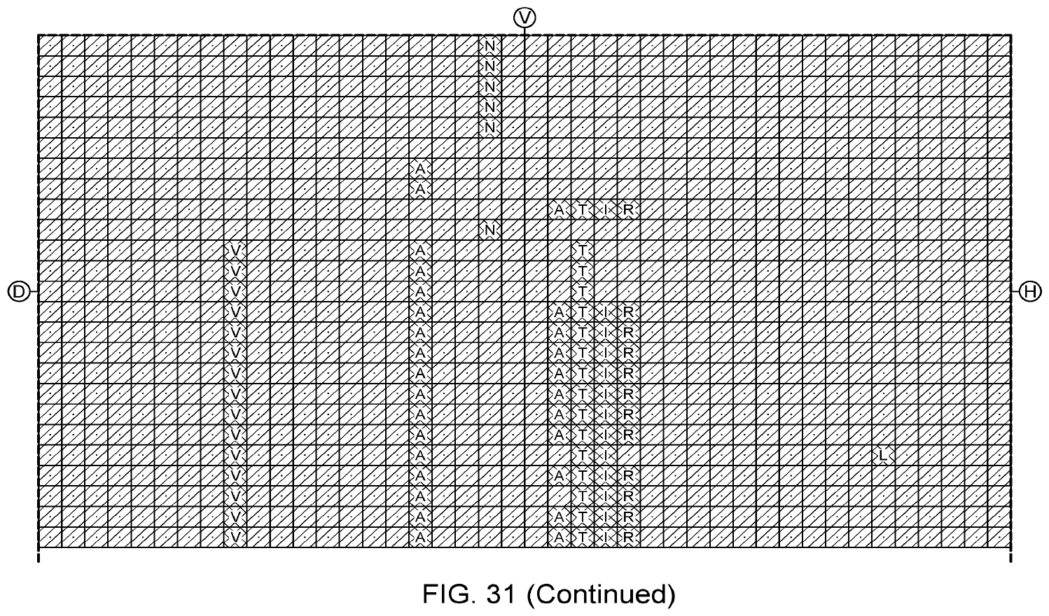

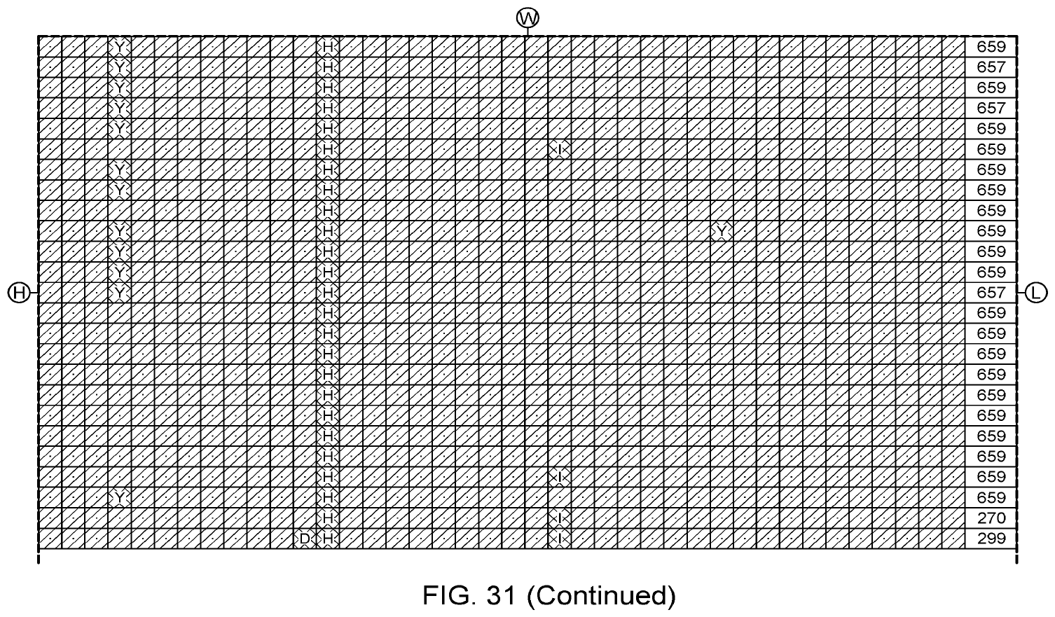

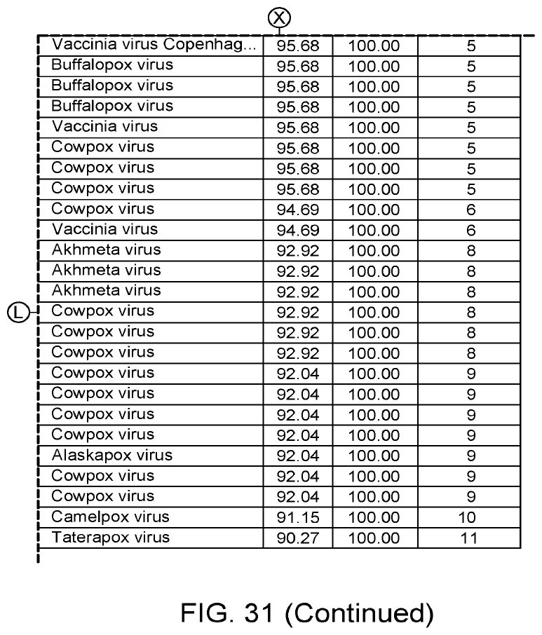

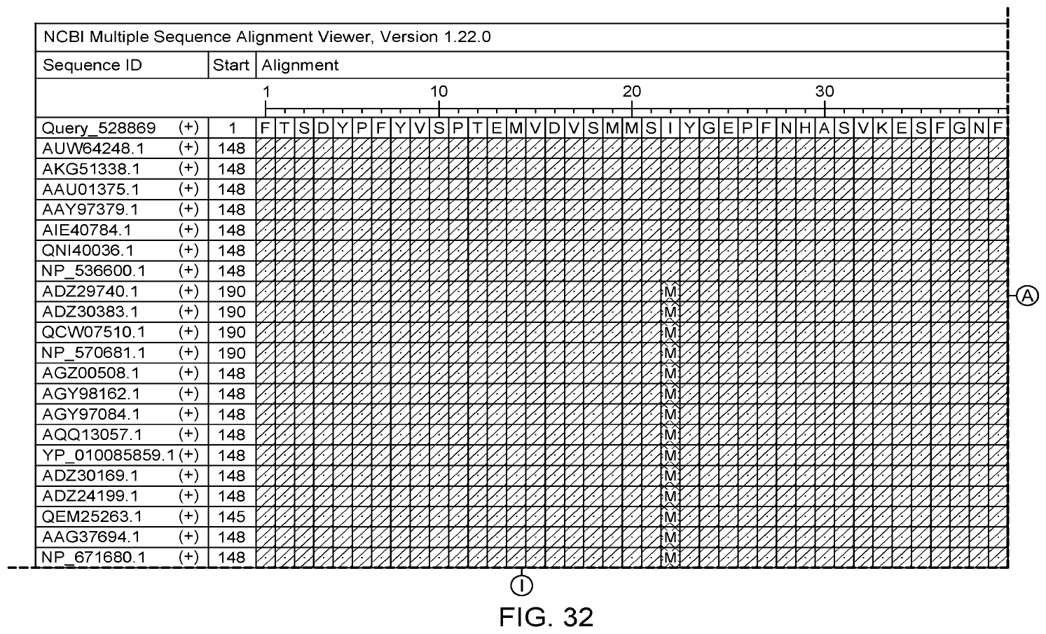

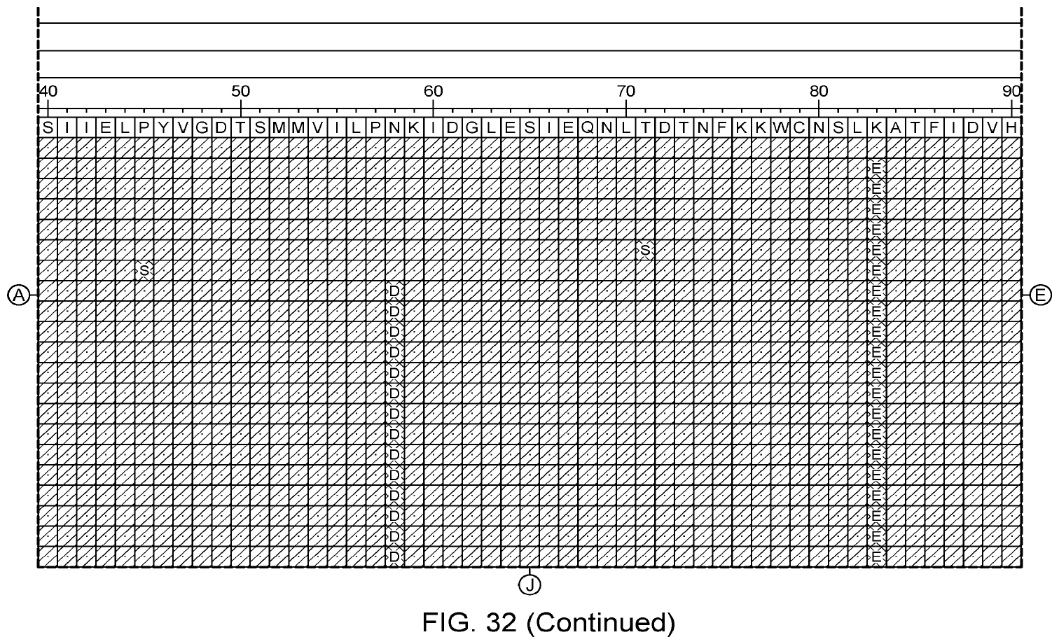

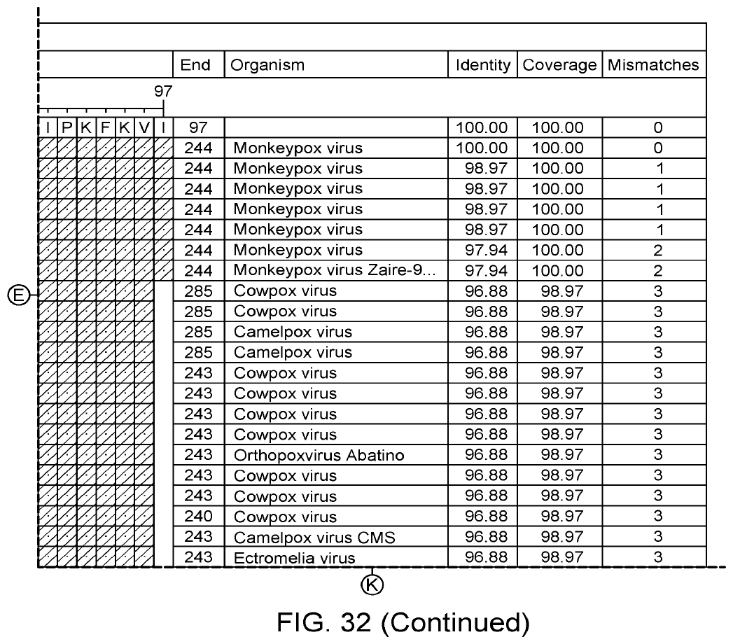

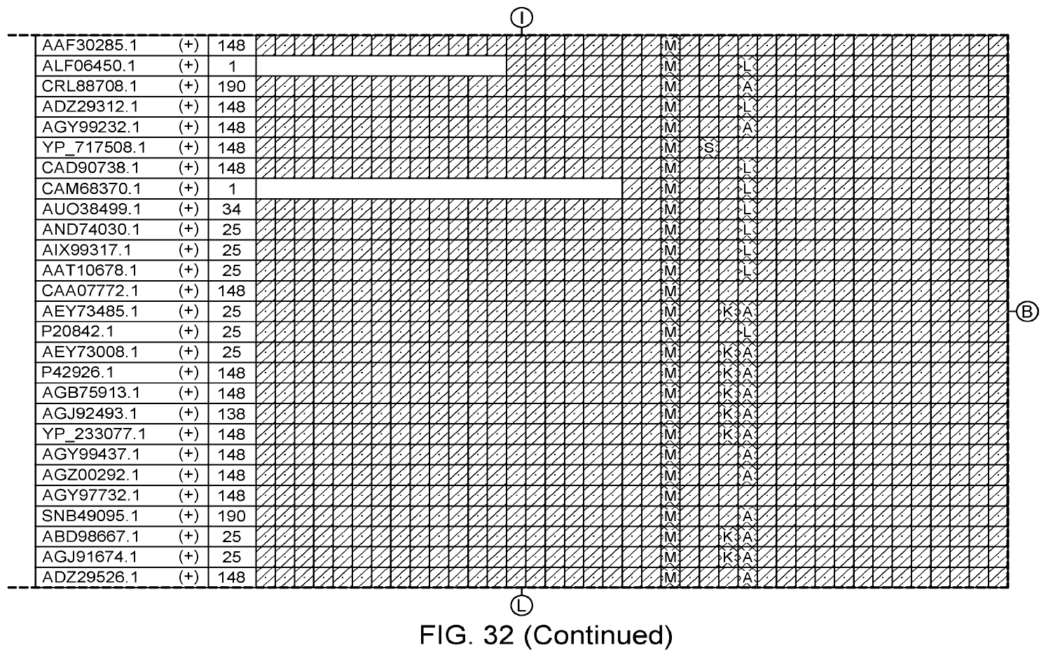

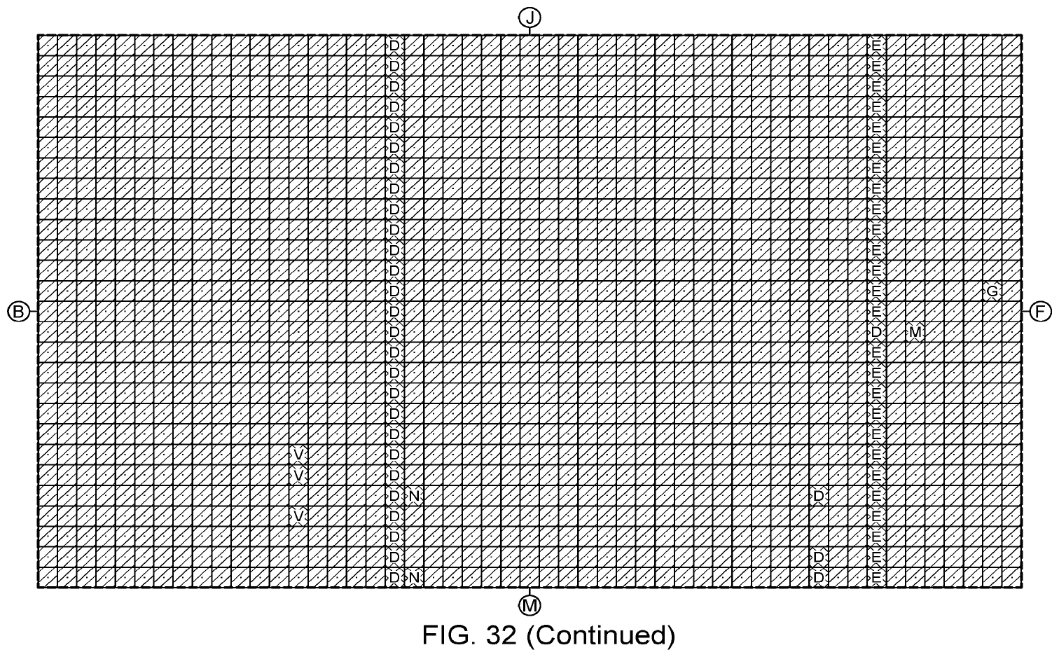

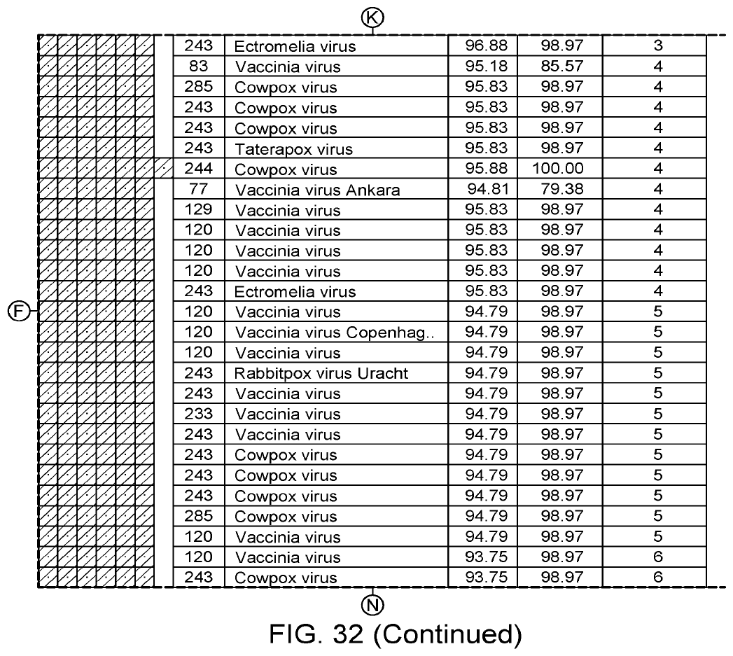

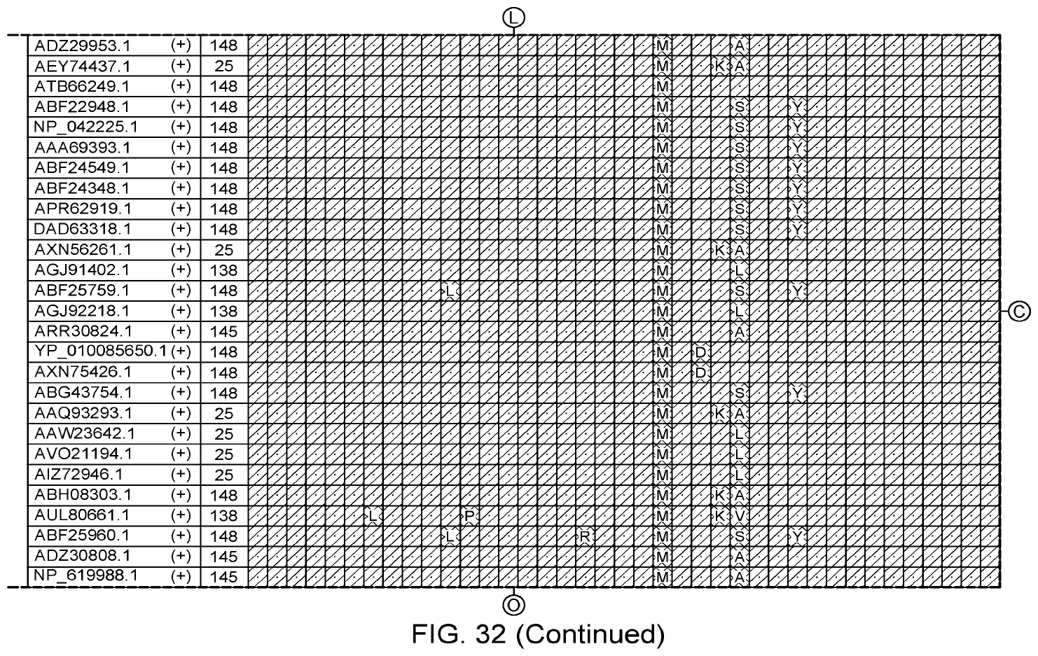

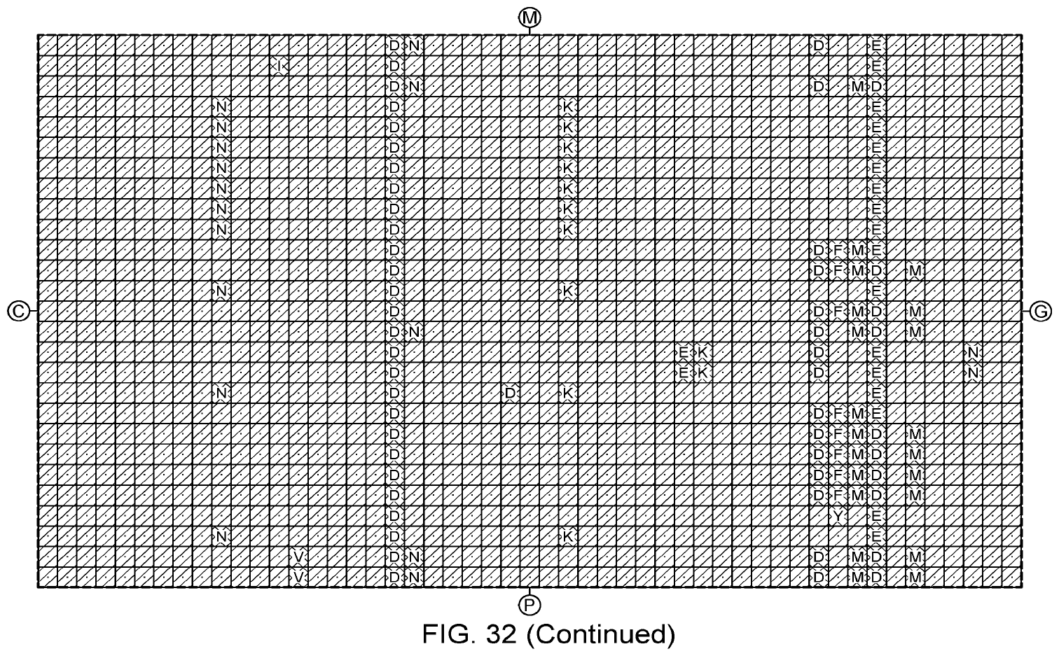

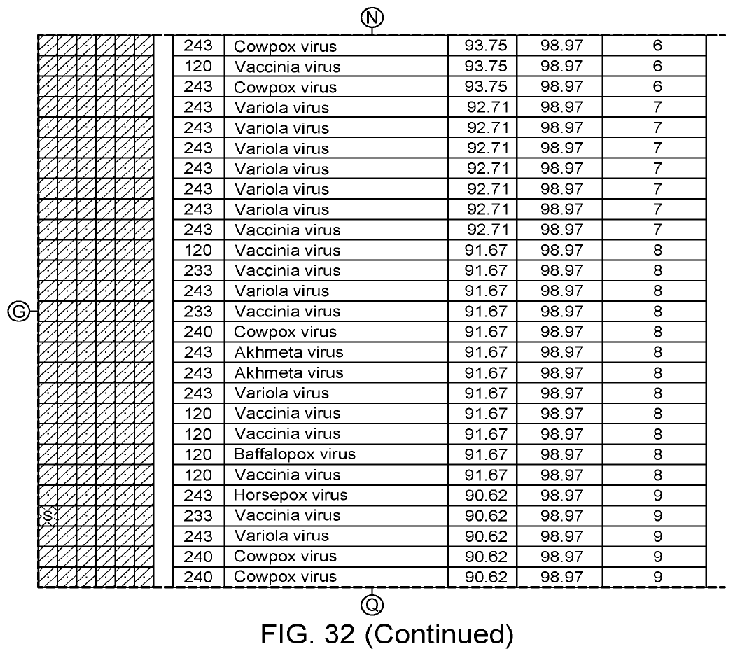

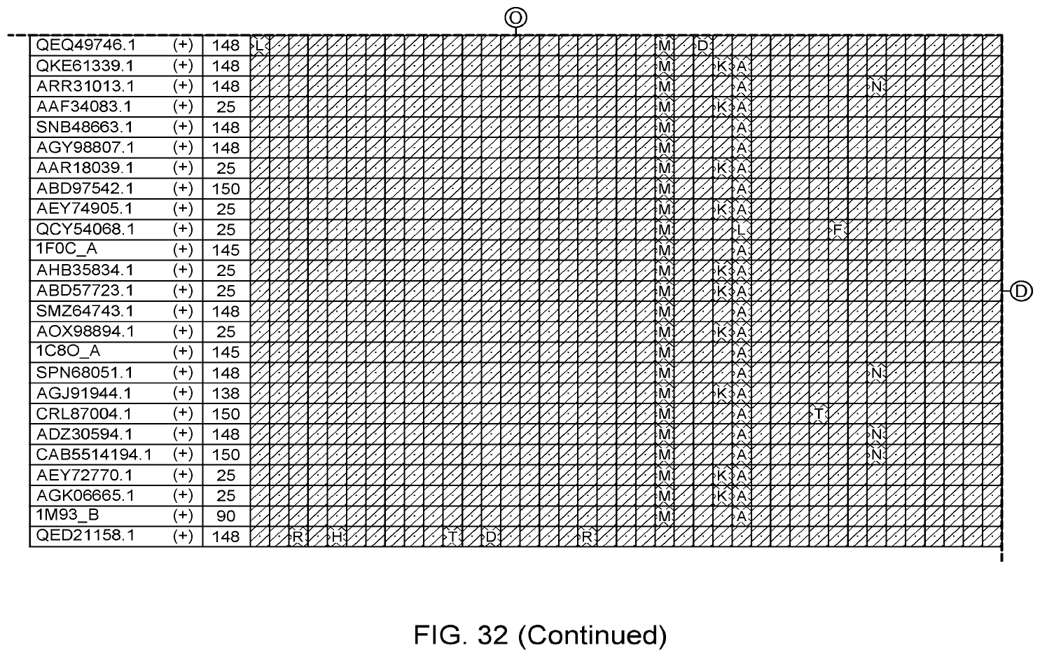

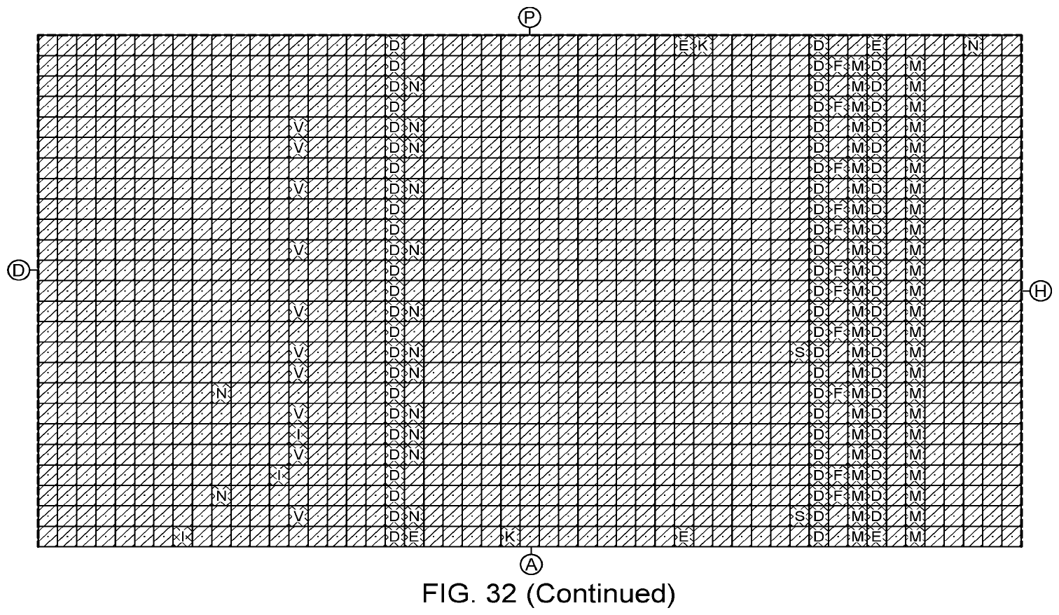

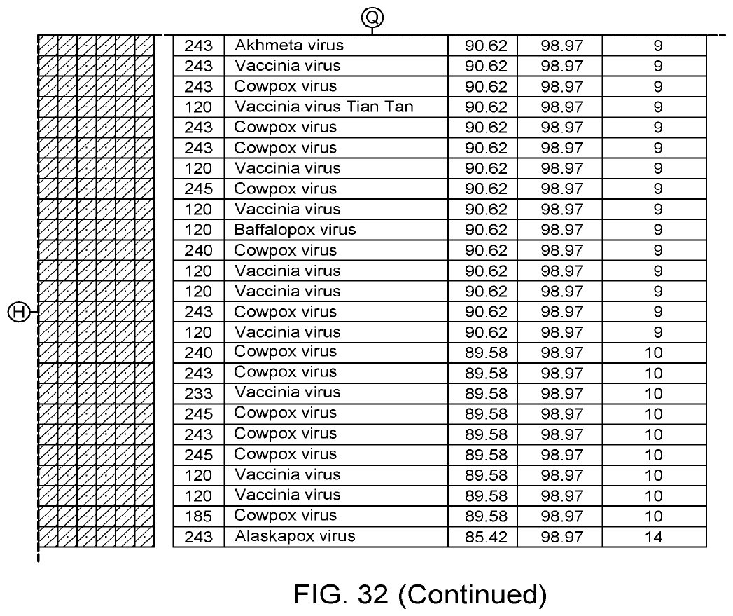

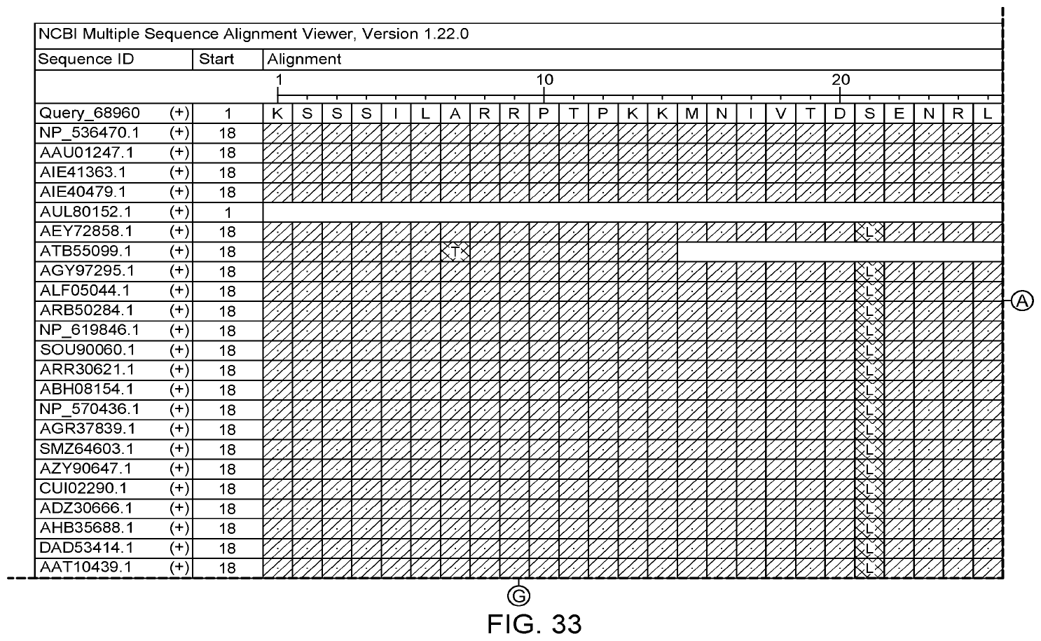

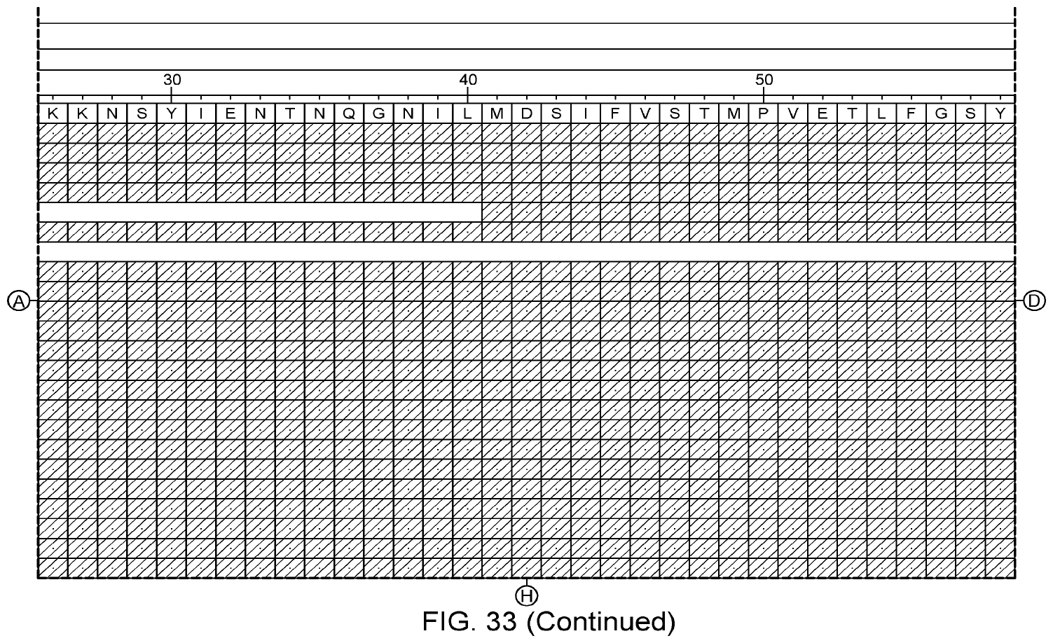

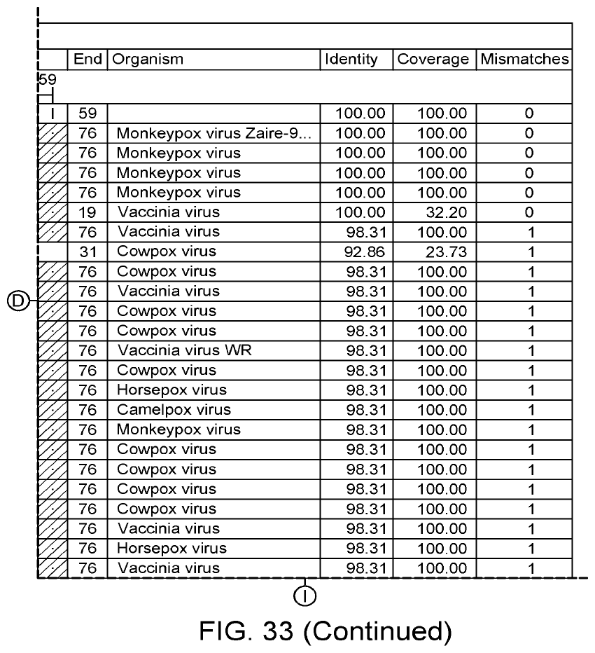

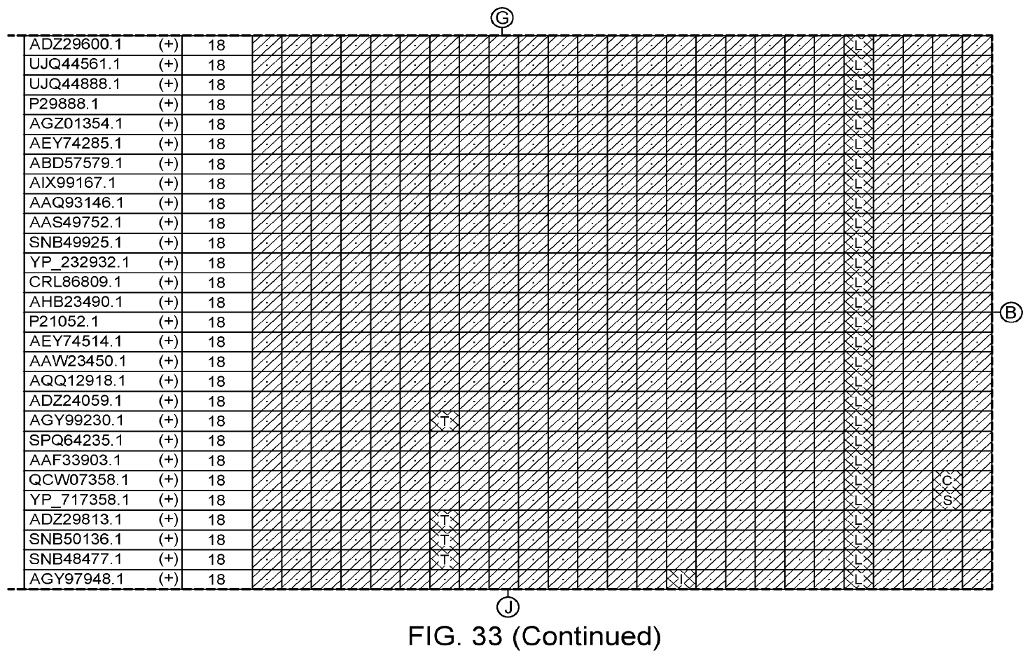

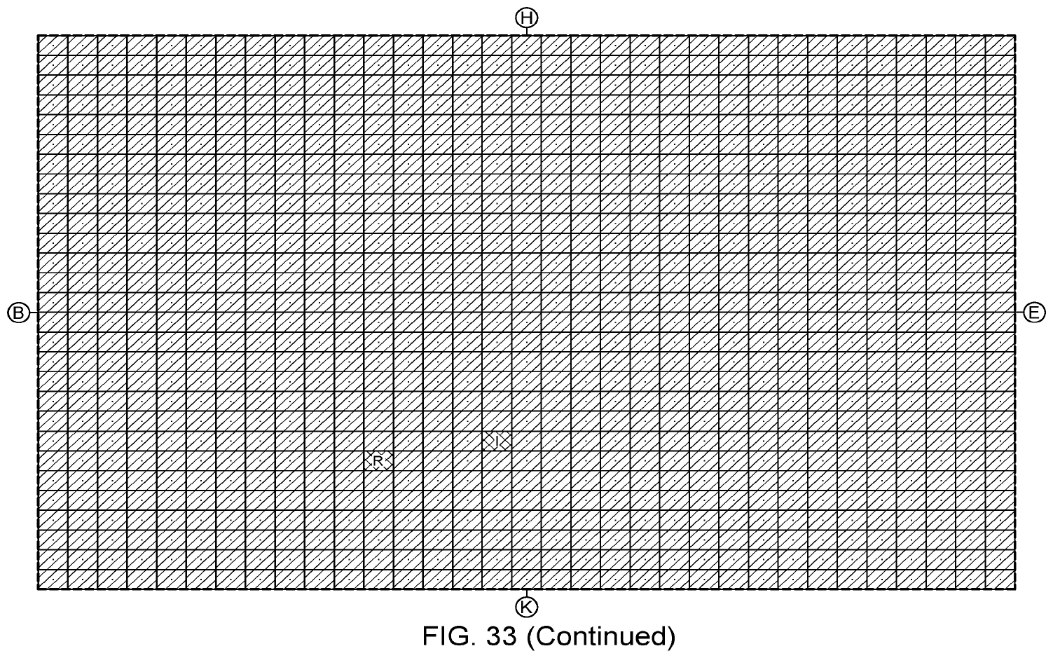

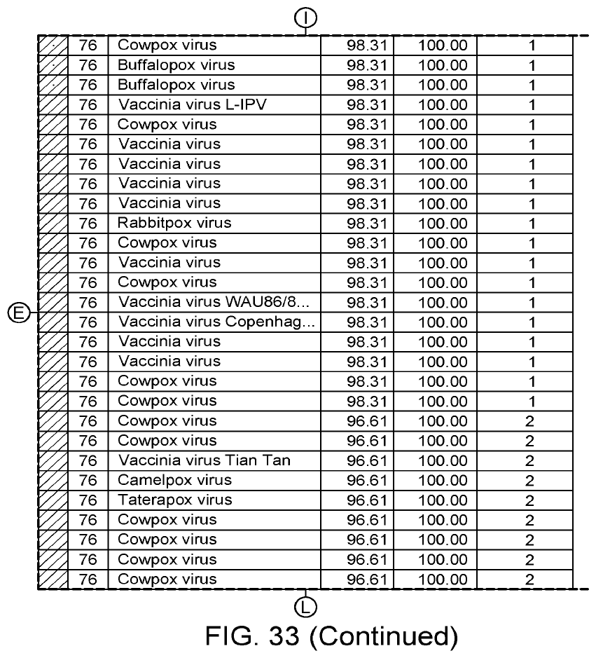

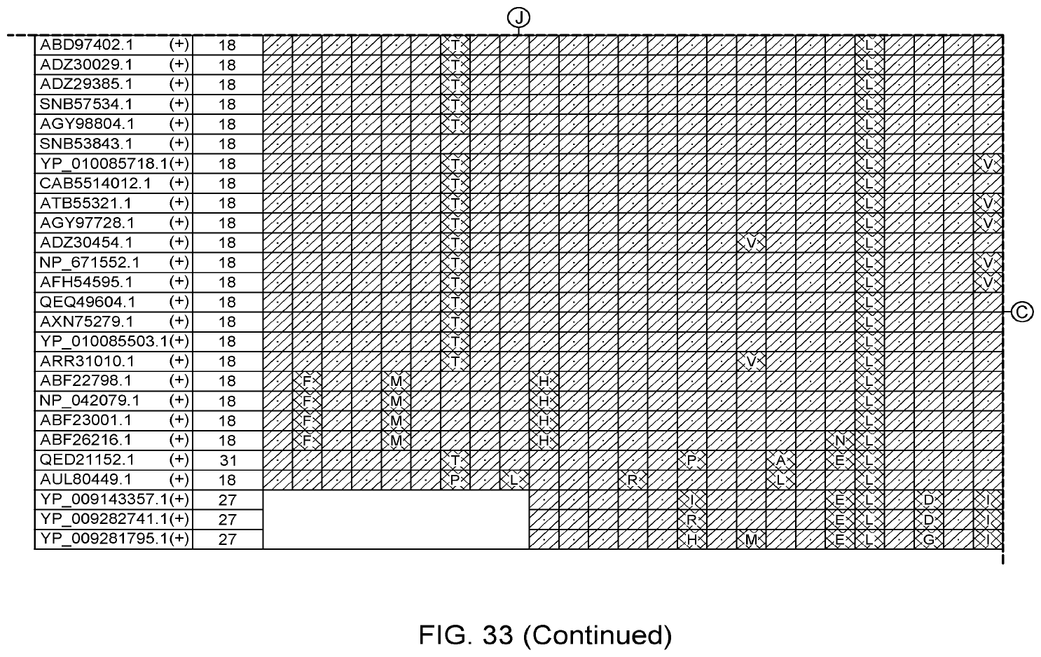

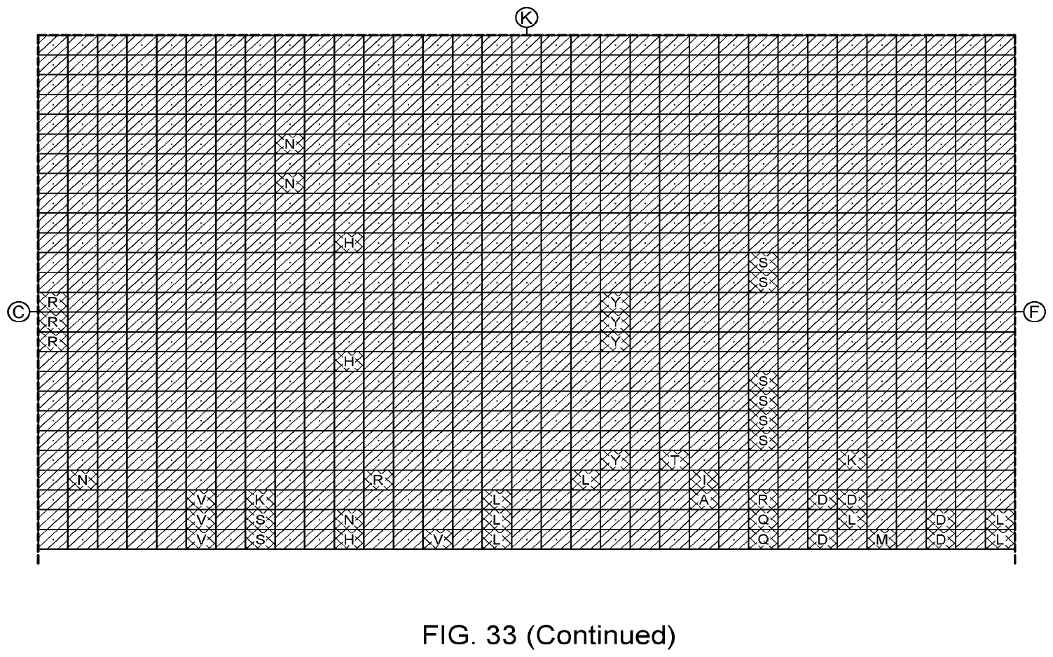

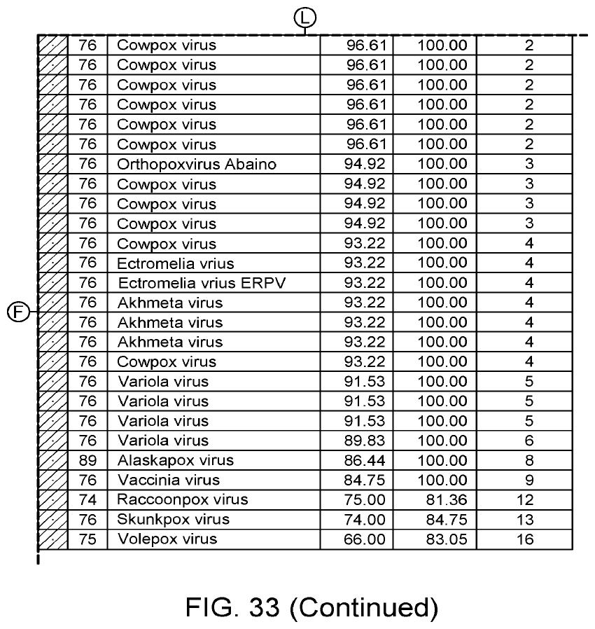

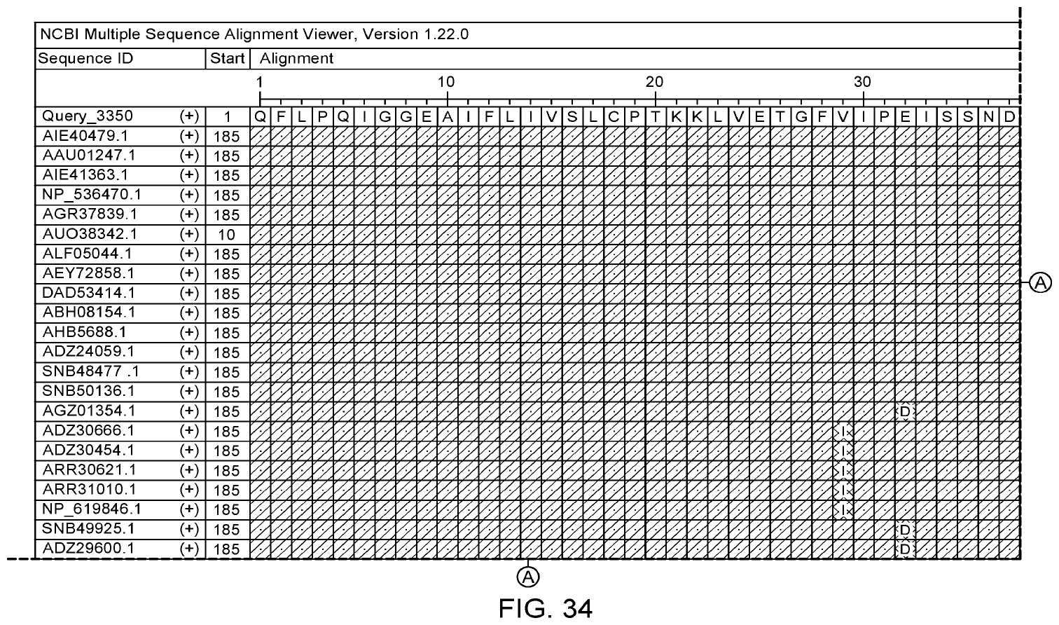

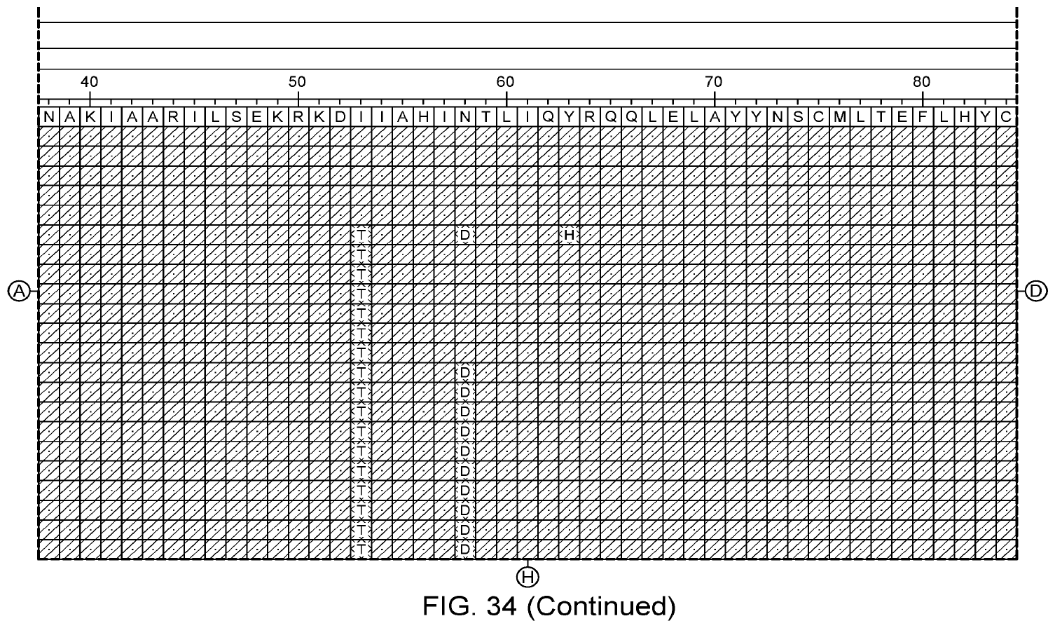

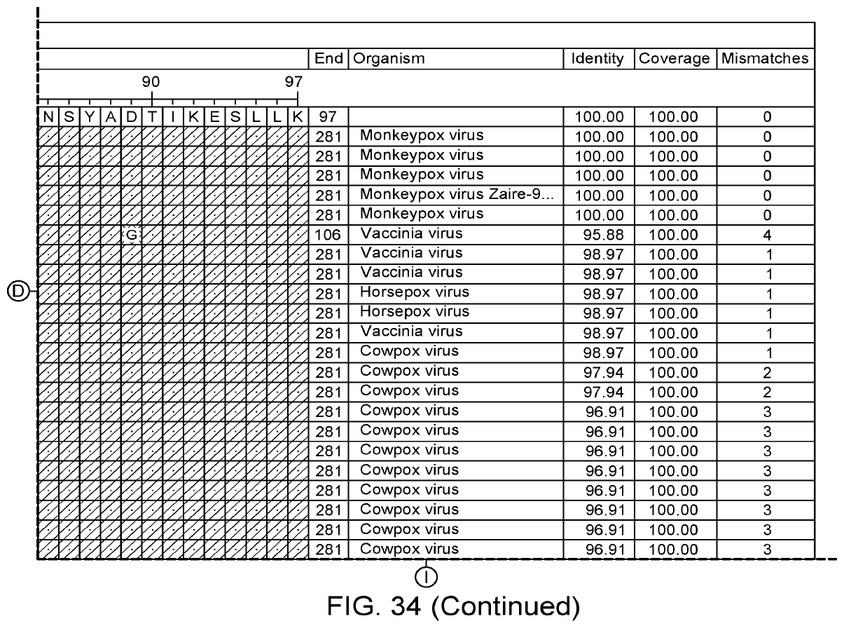

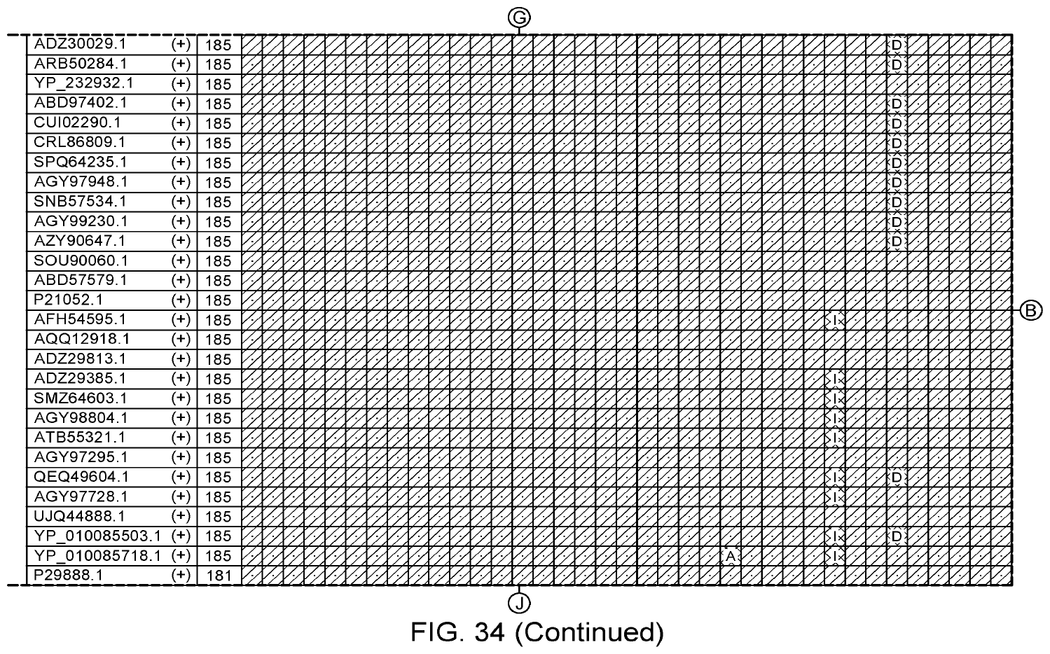

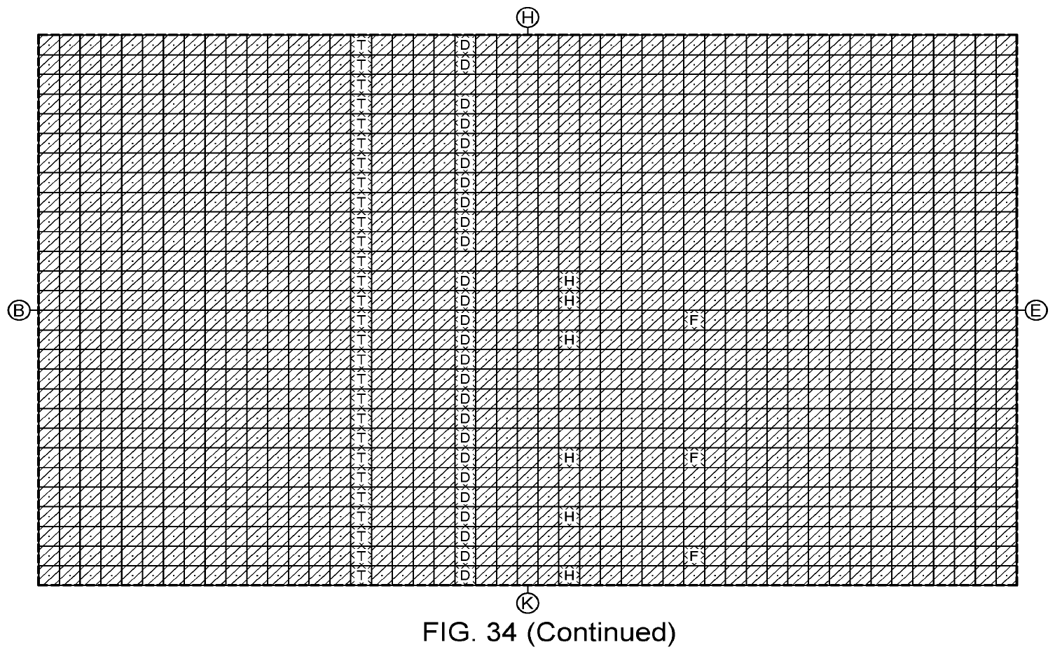

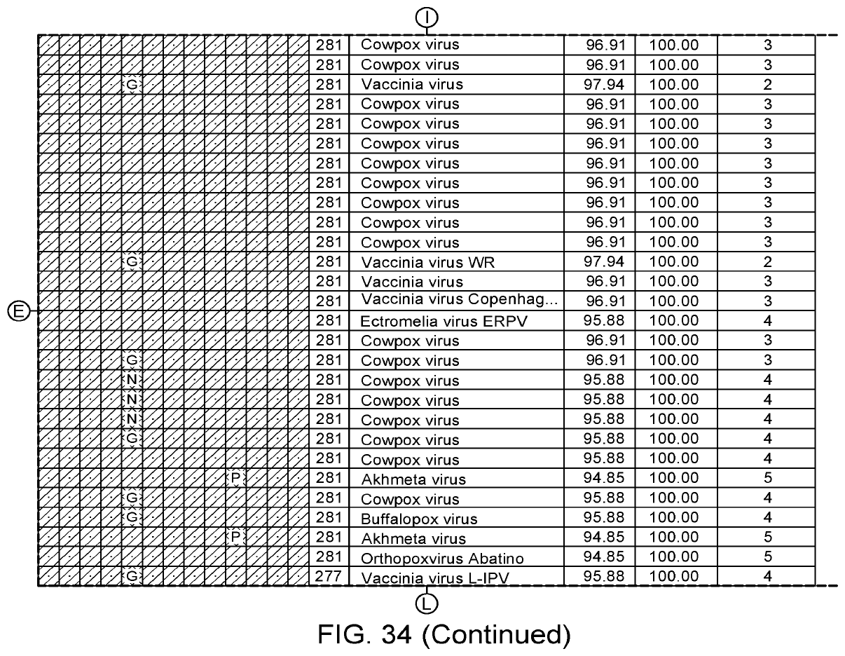

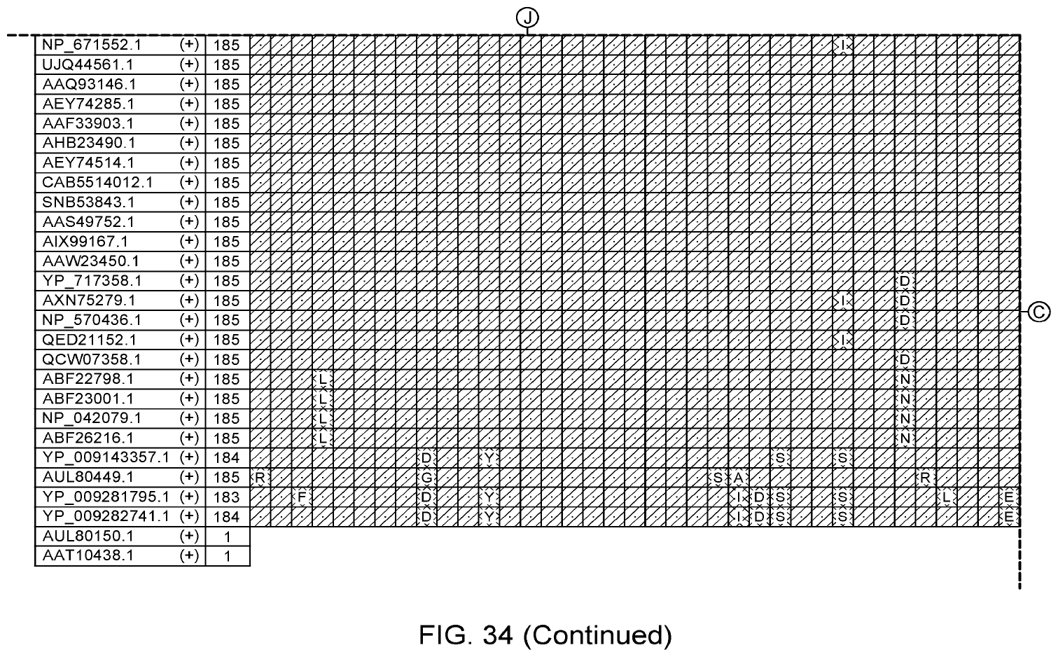

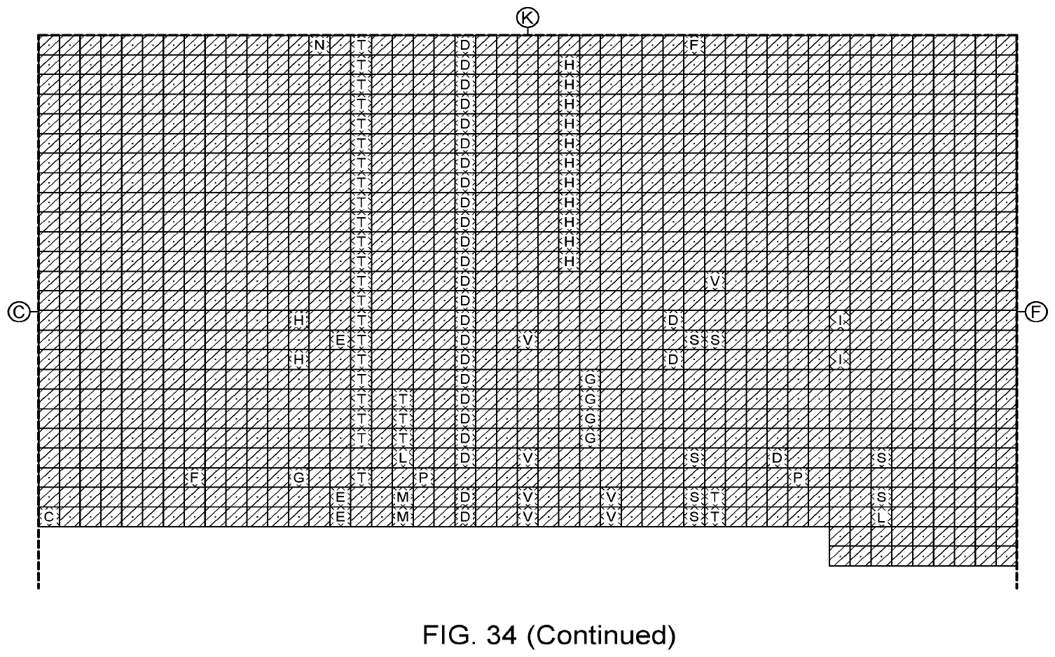

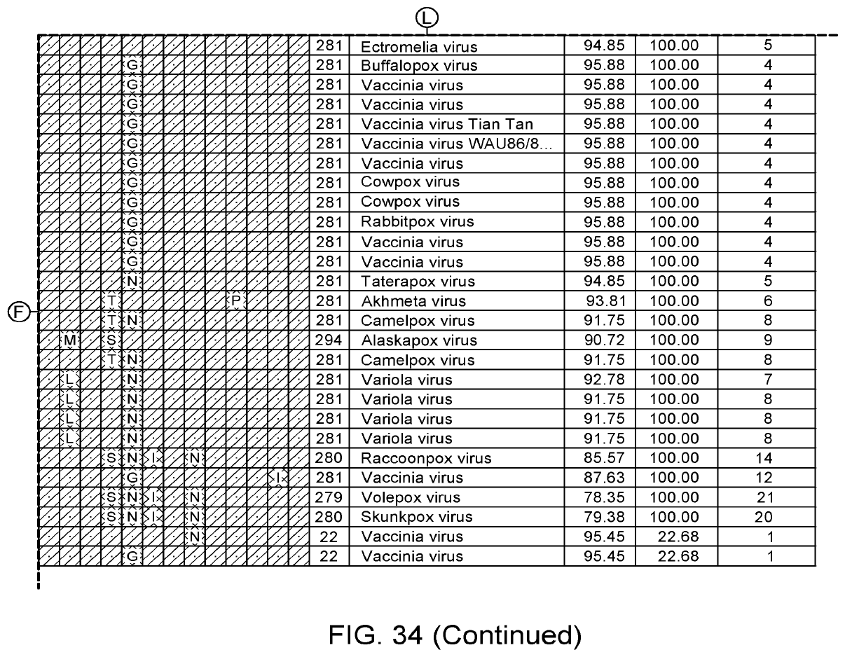

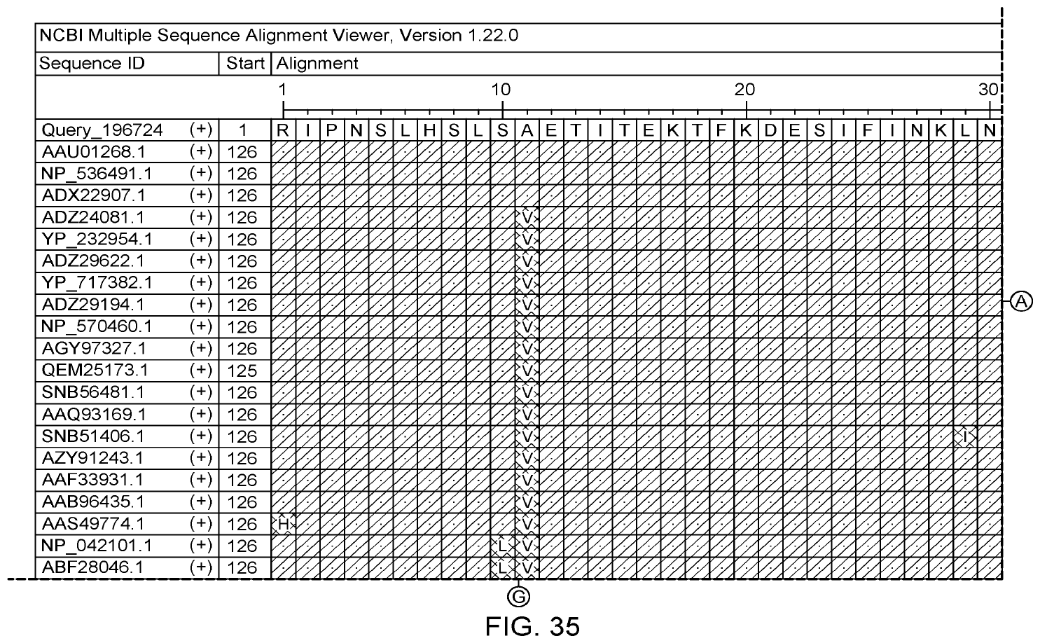

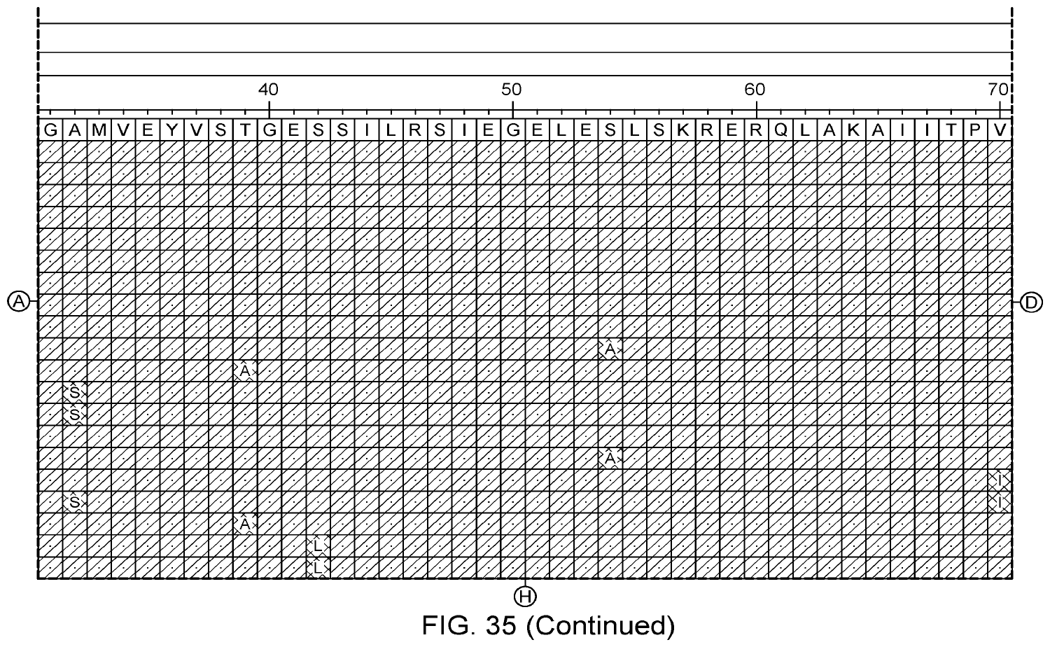

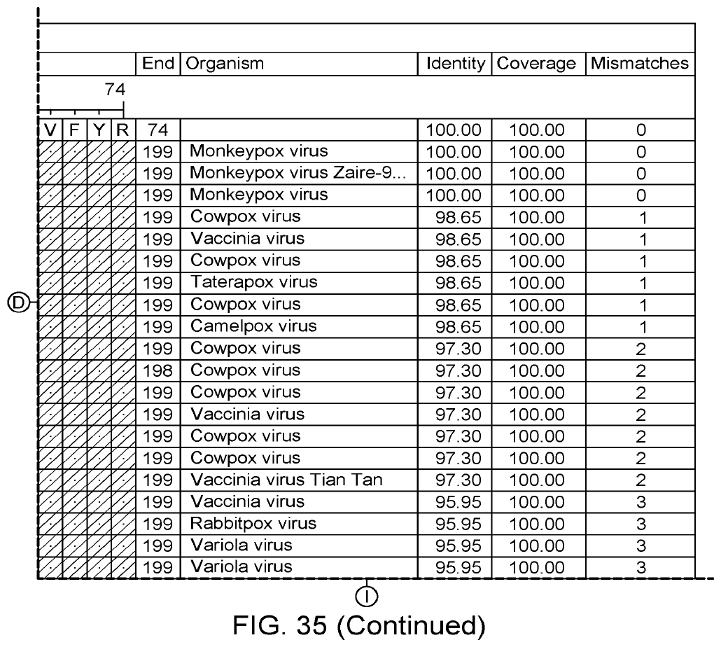

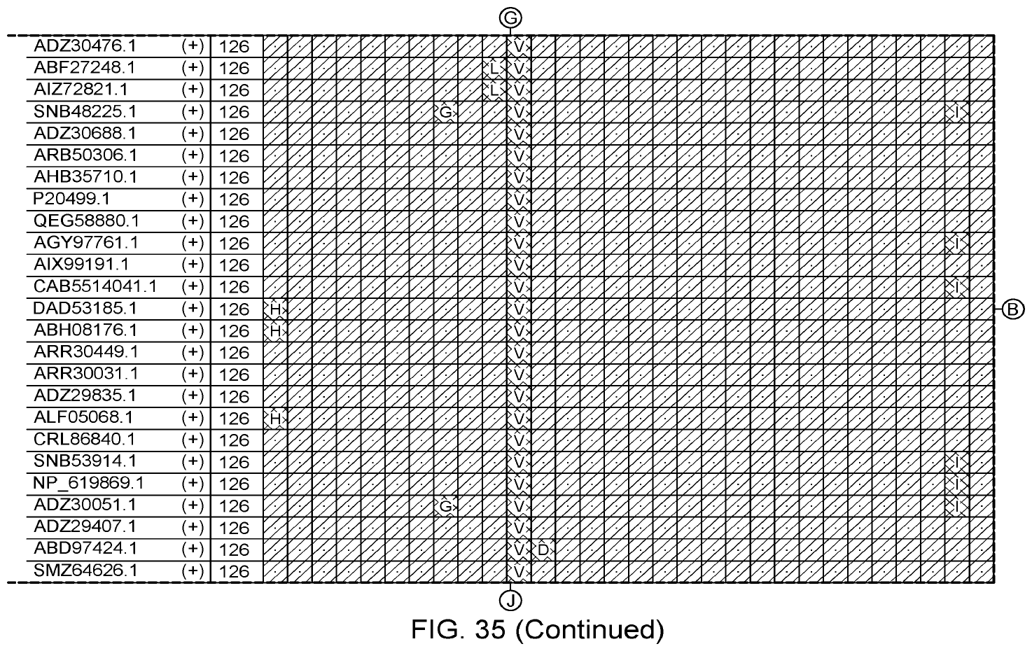

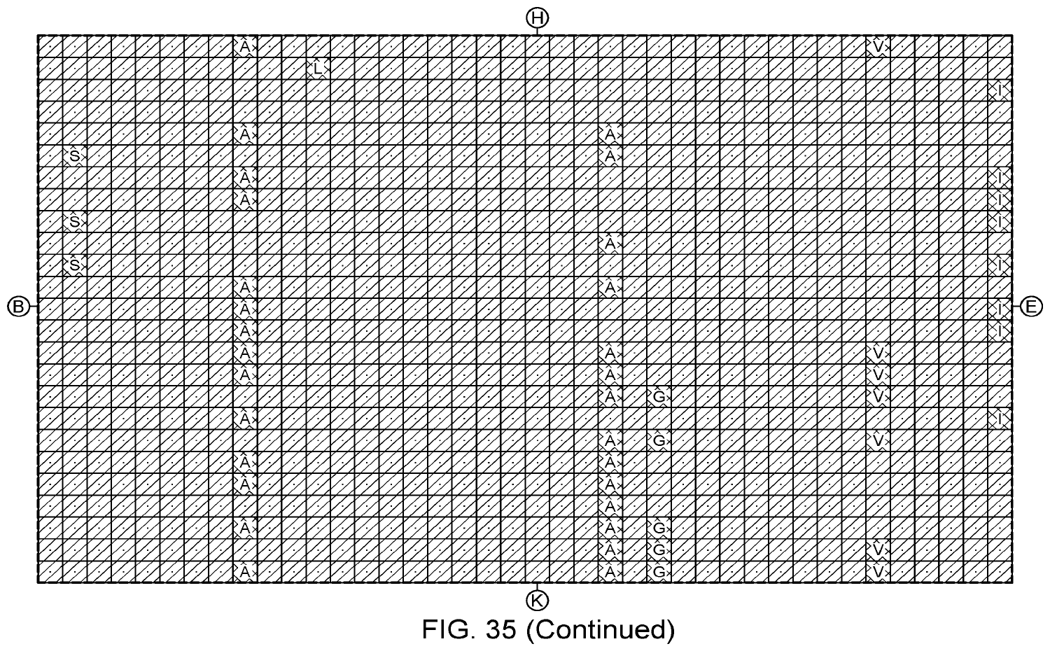

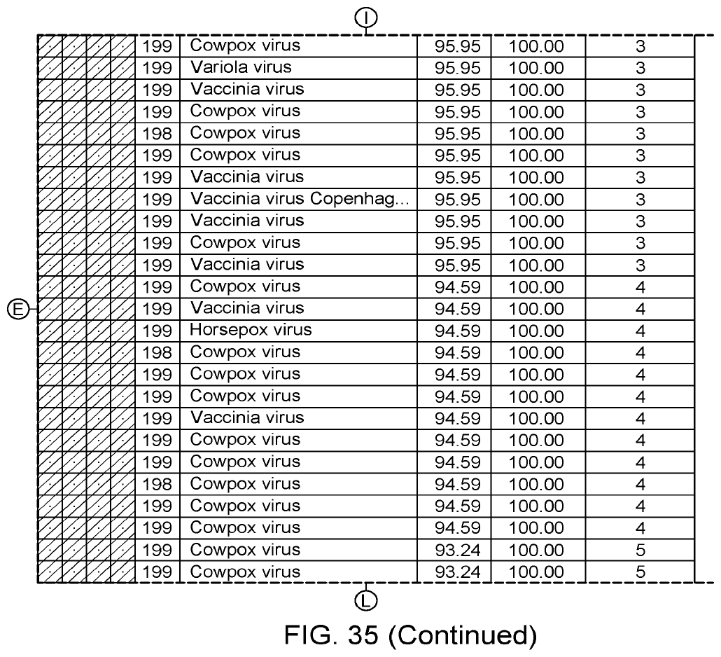

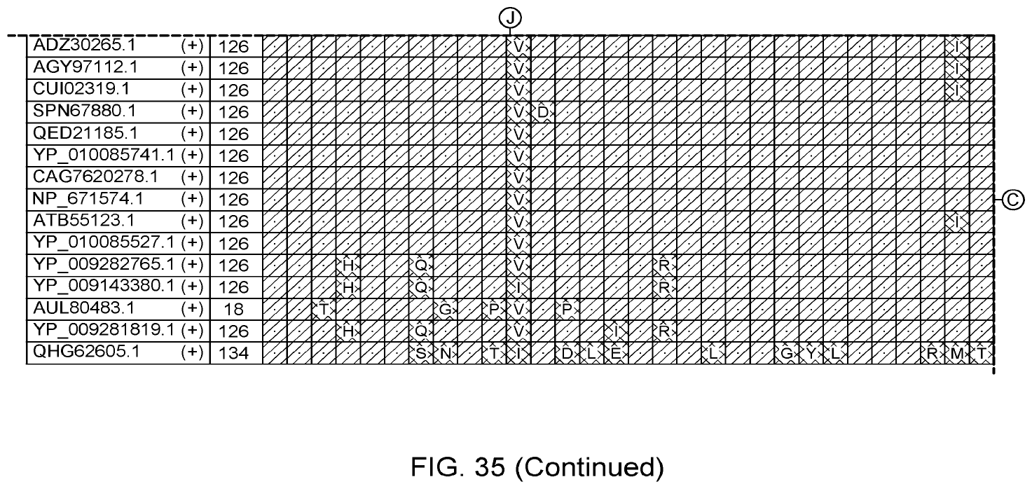

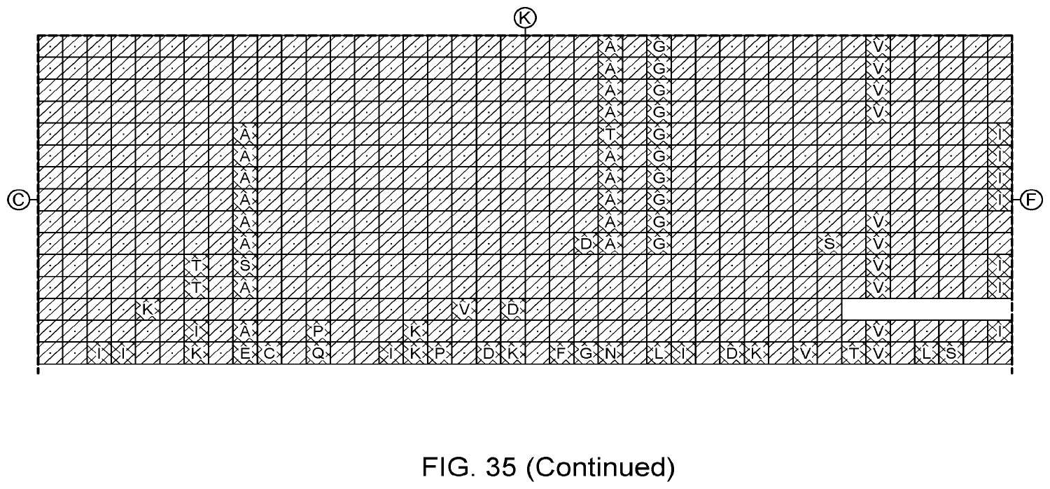

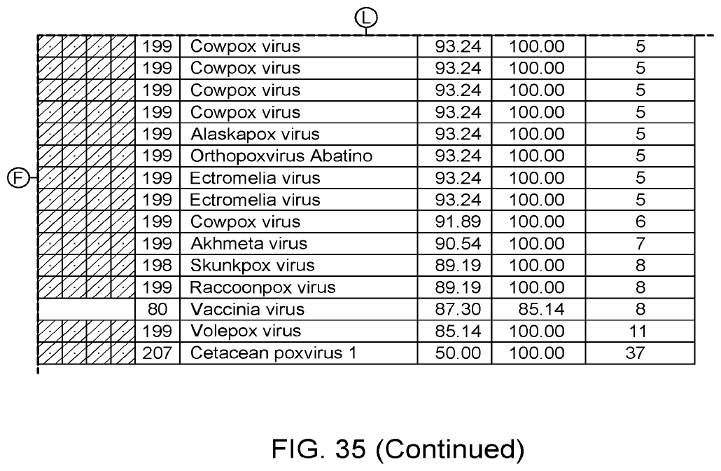

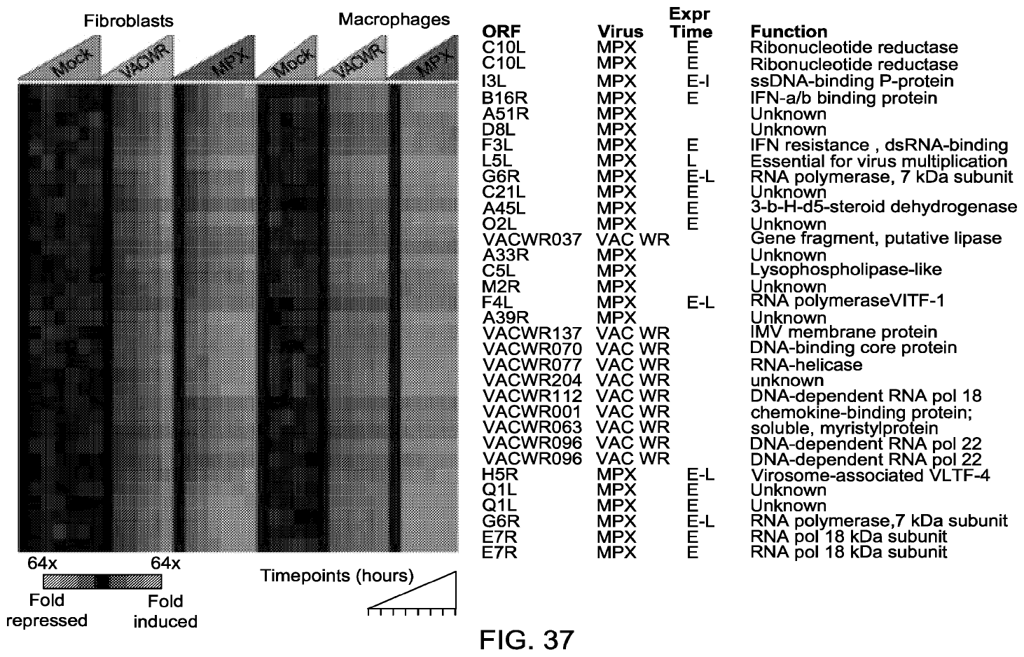

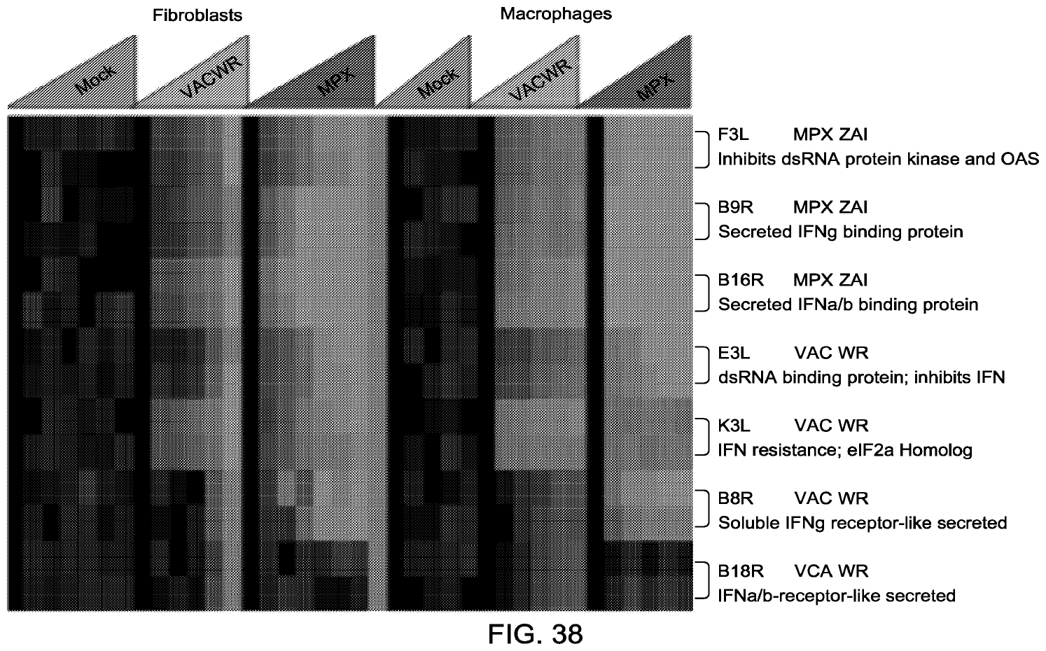

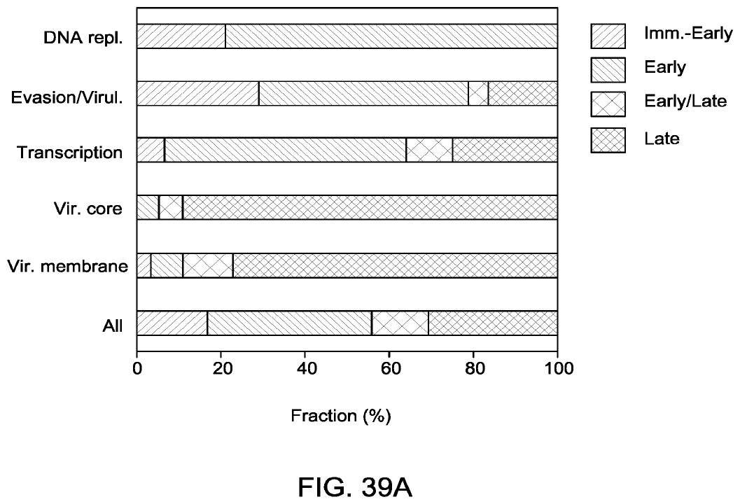

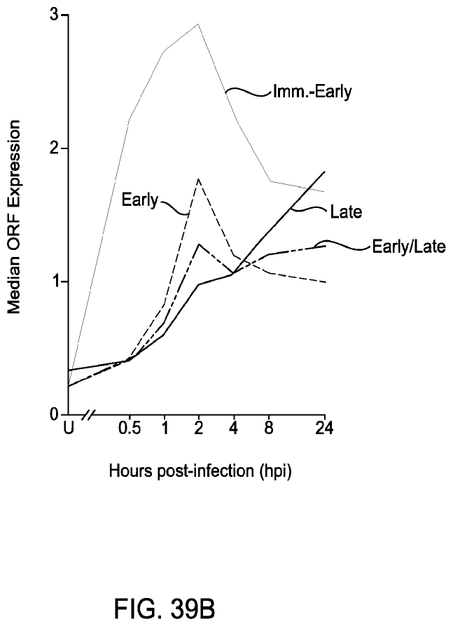

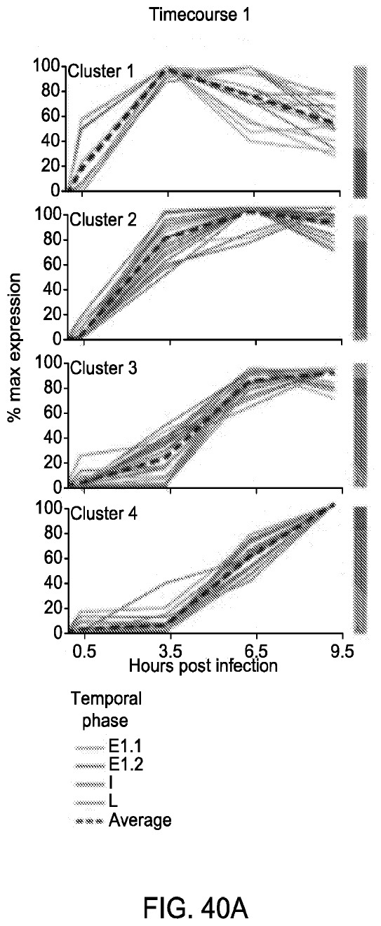

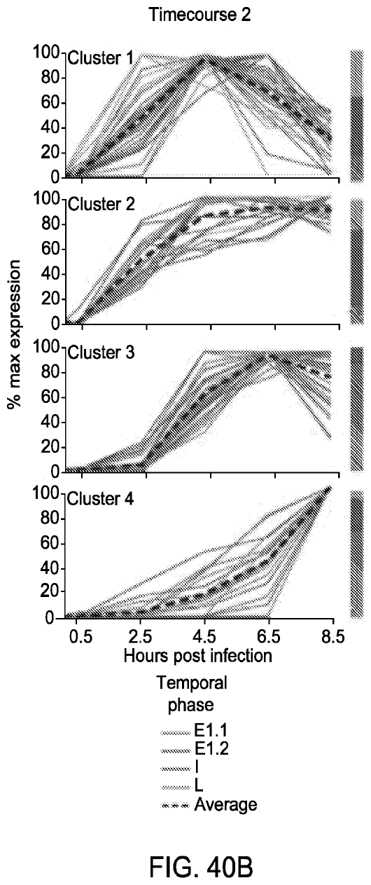

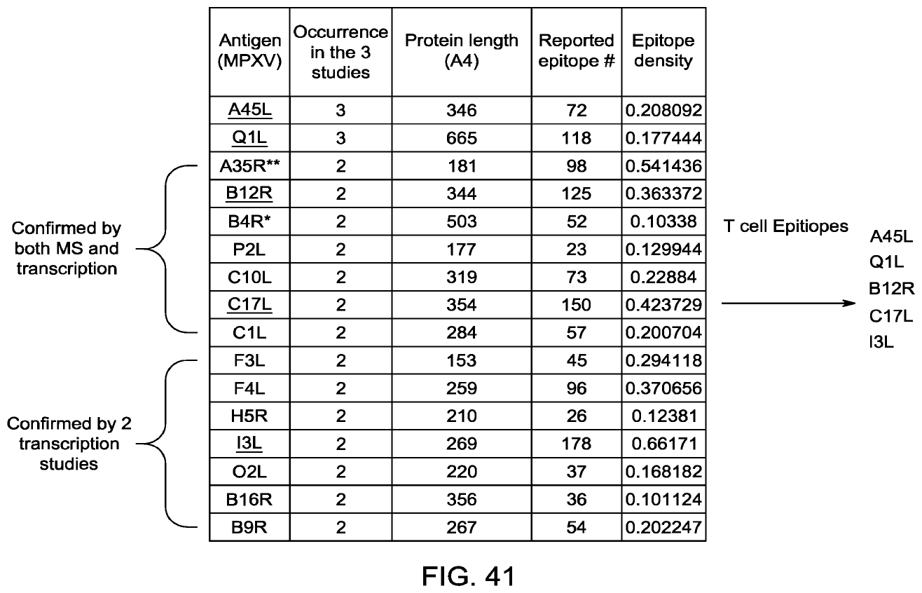

An approach to identifying monkeypox T cell antigens was based on a metaanalysis of diverse sources of information relating to monkeypox gene and/or protein expression. Without wishing to be bound by any particular scientific theory, the present disclosure includes the recognition that early and/or immediately early vaccine products can be preferable for use as antigens at least in part because they permit targeting of monkeypox at early stages of infection and/or moderate the impact of viral immune evasion strategies (which can include suppression of antigen presentation) on vaccine efficacy. One source of information regarding expression of immediate early genes was Rubins et al PlosOne 2008 (DOI: 10.1371/journal.pone.0002628). FIG. 37 and FIG. 38 illustrate gene expression data from Rubins et al PlosOne 2008 (DOI: 10.1371/journal.pone.0002628) that indicates the genes that are expressed most quickly following infection (in particular within 1-2 hours of infection with monkeypox or vaccinia virus), which genes are thereby of interest as T cell antigens. A second source of information regarding expression of immediate early genes was Assarsson et al PNAS 2008 https://doi.org/10.1073/pnas.0711573105. Certain data from Assarsson et al. is shown in FIG. 39A-39B (data relating to immediate early genes are color coded in the Assarsson et al. publication). A third source of information regarding expression of immediate early genes was Croft et al. MCP 2015 (https://doi.org/10.1074/mcp.M114.047373), a proteomics dataset that orthogonally validates transcriptional data. Proteins with greater than 10% maximal expression by 30 minutes were selected as immediate early targets by the present inventors. Certain data from Croft et al. is shown in FIG. 40A-40B. The present disclosure includes that targets identified as early or immediate early in all three of these sources of information, or in both RNA and proteomics information from these sources of information, can be selected as T cell antigens. A further source of information regarding expression of immediate early genes was the IEDB T cell epitope database, which provides eptiopes from literature, filtered for orthopox virus family epitopes and and positive T cell assay results, which filters yielded over 18,000 epitope entries, which were subsequently checked for conservation in monkeypox and/or for presence of a homolog in monkeypox. An analysis of conservation across orthopoxvirus family viruses is shown in FIG. 29 to FIG. 35. Cross-referencing gene expression from all three studies identifies a little of high-confidence IE targets (FIG. 41). Several of these early and/or immediate early viral transcripts encode proteins that are known or predicted to modulate IFN signaling. The present disclosure includes the selection of certain early and/or immediate early monkeypox proteins as T cell antigens, including without limitation A45L, B9R, B16R, C10L, C21L, E7R, F3L, F4L, G6R, H5R, I3L, O2L, Q1L, B12R, and/or C17L.

### Example 2: Exemplary Polyribonucleotide Constructs Encoding Monkeypox Antigens

The present example describes certain exemplary monkeypox antigens, and sequences encoding them, that may be utilized in some embodiments of the present disclosure. Exemplary monkeypox antigens can be found in Tables 1 and 2.

In some particular embodiments, an administered RNA has a structure: Structure 1: m₂^{7,3′-O}Gppp(m₁^{2′-O})ApG-hAg-Kozak-SEC-Immunogen-FI-A30L70, wherein m₂^{7,3′-O}Gppp(m₁^{2′-O})ApG=5′ cap; hAg=5′ UTR human alpha-globin; SEC=signal peptide (SP); Immunogen=a nucleotide sequence comprising a sequence that encodes an antigen described herein; FI=a 3′-UTR that is or comprises a sequence (e.g., 3′ UTR) from the “amino terminal enhancer of split” (AES) messenger RNA and a sequence (e.g., a non-coding region) from the mitochondrial encoded 12S ribosomal RNA (MT-RNR1); and A30L70=a polyA sequence comprising 30 adenine nucleotides followed by 70 adenine nucleotides, wherein the 30 adenine nucleotides and 70 adenine nucleotides are separated by a linker sequence.

In some embodiments, an administered RNA has a structure: Structure 2: m₂^{7,3′-O}Gppp(m₁^{2′-O})ApG-hAg-Kozak-SEC-Immunogen-MITD-FI-A30L70, wherein m₂^{7,3′-O}Gppp(m₁^{2′-O})ApG=5′ cap; hAg=5′ UTR human alpha-globin; SEC=signal peptide (SP); Immunogen=a nucleotide sequence comprising a sequence that encodes one or more antigens described herein; MITD=MHC Class I trafficking signal (MITD); FI=a 3′-UTR that is or comprises a sequence (e.g., 3′ UTR) from the “amino terminal enhancer of split” (AES) messenger RNA and a sequence (e.g., a non-coding region) from the mitochondrial encoded 12S ribosomal RNA (MT-RNR1); and A30L70=a polyA sequence comprising 30 adenine nucleotides followed by 70 adenine nucleotides, wherein the 30 adenine nucleotides and 70 adenine nucleotides are separated by a linker sequence.

### Example 3: Exemplary Polyribonucleotide Constructs Encoding Multiepitope Monkeypox Antigens

The present example describes certain exemplary monkeypox multiepitope antigens, and sequences encoding them, that may be utilized in some embodiments of the present disclosure.

### A) Exemplary Construct Encoding a Monkeypox Multi-Epitope Polypeptide #1

Structure: m₂^{7,3′-O}Gppp(m₁^{2′-O})ApG-hAg-Kozak-SEC-CD8 string-MITD-FI-A30L70

In some embodiments, a CD8 string may comprise sequences that encode at least 2 (including, e.g., at least 1, at least 2, at least 3, at least 4, at least 5, at least 10, at least 15, at least 20, or more) CD8+ T cell epitopes and/or HLA-I epitopes of antigens listed in Table 2, or fragments thereof. In some embodiments, a CD8 string may comprise a sequence that encodes an antigen/epitope having an amino acid sequence as recited in Table 2, or a fragment thereof.

### B) Exemplary Construct Encoding a Monkeypox Multi-Epitope Polypeptide #2

Structure m₂^{7,3′-O}Gppp(m₁^{2′-O})ApG-hAg-Kozak-SEC-CD4 string-MITD-FI-A30L70

In some embodiments, a CD4 string may comprise sequences that encode at least 2 (including, e.g., at least 1, at least 2, at least 3, at least 4, at least 5, at least 10, at least 15, at least 20, or more) CD4+ T cell epitopes and/or HLA-II epitopes of antigens listed in Table 2, or fragments thereof. In some embodiments, a CD4 string may comprise a sequence that encodes an antigen/epitope having an amino acid sequence as recited in Table 2, or a fragment thereof.

### C) Exemplary Construct Encoding a Monkeypox Multi-Epitope Polypeptide #3

Structure m₂^{7,3′-O}Gppp(m₁^{2′-O})ApG-hAg-Kozak-SEC-CD8 chunk sequence-MITD-FI-A30L70

In some embodiments, a CD8 chunk sequence may comprise at least 2 (including, e.g., at least 1, at least 2, at least 3, at least 4, at least 5, at least 10, at least 15, at least 20, or more) immunogenic fragment(s) of polypeptide(s) encoded by gene(s) or peptide(s) as listed in Table 2, or fragments thereof. In some embodiments, a sequence can comprise an SEC, CD8 chunk sequence, and MITD.

### D) Exemplary Construct Encoding a Monkeypox Multi-Epitope Polypeptide #4

Structure m₂^{7,3′-O}Gppp(m₁^{2′-O})ApG-hAg-Kozak-SEC-CD4 chunk sequence-MITD-FI-A30L70

In some embodiments, a CD4 chunk sequence may comprise at least 2 (including, e.g., at least 1, at least 2, at least 3, at least 4, at least 5, at least 10, at least 15, at least 20, or more) immunogenic fragment(s) of polypeptide(s) encoded by gene(s) or peptide(s) as listed in Table 2, or fragments thereof. In some embodiments, a sequence can comprise an SEC, CD4 chunk sequence, and MITD.

### Example 4: Exemplary LNP Formulations

The present Example describes certain preferred LNP formulations useful for vaccine compositions as described herein.

In some embodiments, LNP formulations that are useful for vaccine compositions as described herein can comprise at least one ionizable aminolipid. In some embodiments, LNP formulations that are useful for vaccine compositions as described herein can further comprise a helper lipid, which in some embodiments may be or comprise a neutral helper lipid. In some embodiments, LNP formulations that are useful for vaccine compositions as described herein can further comprise a polymer-conjugated lipid, for example in some embodiments PEG-conjugated lipids. In some embodiments, LNP formulations that are useful for vaccine compositions as described herein can comprise at least one ionizable aminolipid, at least one helper lipid (e.g., a neutral helper lipid, which in some embodiments may comprise a phospholipid, a steroid, or combinations thereof), and at least one polymer-conjugated lipid (e.g., PEG-conjugated lipid). In some embodiments, an exemplary LNP formulation may comprise an ionizable aminolipid, a phospholipid, a steroid, and a PEG-conjugated lipid.

In some embodiments, an ionizable aminolipid may be present in an LNP formulation within a range of 45 to 55 mol percent, 40 to 50 mol percent, 41 to 49 mol percent, 41 to 48 mol percent, 42 to 48 mol percent, 43 to 48 mol percent, 44 to 48 mol percent of total lipids. In some embodiments, an exemplary ionizable aminolipid is or comprises ((4-hydroxybutyl)azanediyl)bis(hexane-6,1-diyl)bis(2-hexyldecanoate) (also known as 6-[N-6-(2-hexyldecanoyloxy)hexyl-N-(4-hydroxybutyl)amino]hexyl 2-hexyldecanoate). In some embodiments, an exemplary ionizable aminolipid is or comprises SM-102 (heptadecan-9-yl 8 ((2 hydroxyethyl)(6 oxo 6-(undecyloxy)hexyl)amino)octanoate) or an aminolipid as described in Sabnis et al. “A Novel Amino Lipid Series for mRNA Delivery: Improved Endosomal Escape and Sustained Pharmacology and Safety in Non-human Primates” Mol. Ther. (2018) 26:1509-1519. In some embodiments, an exemplary ionizable aminolipid is or comprises an ionizable aminolipid as disclosed in US2020/0163878 or WO2018/078053, the entire contents of each of which are incorporated herein by reference for the purposes described herein.

In some embodiments, a phospholipid may be present in an LNP formulation within a range of 5 to 15 mol percent, 7 to 13 mol percent, or 9 to 11 mol percent of total lipids. In some embodiments, an exemplary phospholipid is or comprises 1,2-Distearoyl-sn-glycero-3-phosphocholine (DSPC).

In some embodiments, a sterool may be present in an LNP formulation within a range of 30 to 50 mol percent, 35 to 45 mol percent or 38 to 43 mol percent of total lipids. In some embodiments, an exemplary sterol is or comprises cholesterol.

In some embodiments, a polymer conjugated lipid (e.g., PEG-conjugated lipid) may be present in an LNP formulation within a range of 1 to 10 mol percent, 1 to 5 mol percent, or 1 to 2.5 mol percent of total lipids. In some embodiments, an exemplary PEG-conjugated lipid is or comprises 2-[(polyethylene glycol)-2000]—N,N-ditetradecylacetamide (also known as 2-[2-(ω-methoxy (polyethyleneglycol2000) ethoxy]-N,N-ditetradecylacetamide). In some embodiments, an exemplary phospholipid is or comprises PEG2000-DMG (1-monomethoxypolyethyleneglycol-2,3-dimyristylglycerol with polyethylene glycol of average molecular weight 2000). In some embodiments, an exemplary PEG-conjugated lipid is or comprises a PEG-lipid as disclosed in US2020/0163878 or WO2018/078053, the entire contents of each of which are incorporated herein by reference for the purposes described herein.

In some embodiments, an exemplary LNP formulation comprises (i) an ionizable aminolipid within a range of 45 to 55 mol percent of total lipids; (ii) a phospholipid within a range of 8 to 12 mol percent of total lipids; (iii) a steroid within a range of 35 to 45 mol percent of total lipids; and (iv) a polymer conjugated (e.g., PEG-conjugated polymer) within a range of 1 to 2 mol percent of total lipids; and RNA molecules as described herein that are encapsulated within or associated with the lipid nanoparticles.

In some embodiments, an exemplary LNP formulation comprises (i) ionizable amino lipid within a range of 45 to 55 mol percent of total lipids; (ii) DSPC within a range of 5 to 15 mol percent of total lipids; (iii) cholesterol within a range of 35 to 45 mol percent of total lipids; and (iv) a PEG-conjugated lipid within a range of 1 to 2 mol percent of total lipids; and RNA molecules as described herein that are encapsulated within or associated with the lipid nanoparticles.

In some embodiments, an exemplary LNP formulation comprises (i) an ionizable aminolipid within a range of 40 to 50 mol percent of total lipids; (ii) a phospholipid within a range of 5 to 15 mol percent of total lipids; (iii) a steroid within a range of 35 to 45 mol percent of total lipids; and (iv) a polymer conjugated (e.g., PEG-conjugated polymer) within a range of 1 to 10 mol percent of total lipids; and RNA molecules as described herein that are encapsulated within or associated with the lipid nanoparticles. In some such embodiments, an ionizable aminolipid is or comprises ((4-hydroxybutyl)azanediyl)bis(hexane-6,1-diyl)bis(2-hexyldecanoate) (also known as 6-[N-6-(2-hexyldecanoyloxy)hexyl-N-(4-hydroxybutyl)amino]hexyl 2-hexyldecanoate). In some such embodiments, a phospholipid is or comprises 1,2-Distearoyl-sn-glycero-3-phosphocholine (DSPC). In some such embodiments, a steroid is or comprises cholesterol. In some such embodiments, a polymer conjugated polymer is or comprises 2-[(polyethylene glycol)-2000]—N,N-ditetradecylacetamide (also known as 2-[2-(ω-methoxy (polyethyleneglycol2000) ethoxy]-N,N-ditetradecylacetamide).

In one embodiment, an exemplary LNP formulation comprises the following lipids included in Table 6 below and RNA molecules as described herein.

TABLE 6
Exemplary LNP Formulation

| | Proportion |
|---|---|
| Lipids | (mol %) |
| ((4-hydroxybutyl)azanediyl)bis(hexane-6,1-diyl)bis(2- | 47-49 (e.g., 47.5) |
| hexyldecanoate) (also known as 6-[N-6-(2- | |
| hexyldecanoyloxy)hexyl-N-(4-hydroxybutyl)amino]hexyl 2- | |
| hexyldecanoate) | |
| 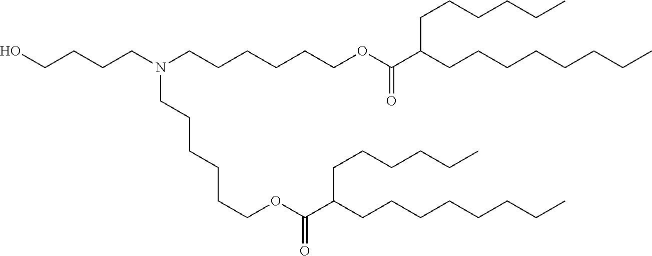 [SMILES: CCCCCCCCC(CCCCCC)C(=O)OCCCCCCN(CCCCO)CCCCCCOC(=O)C(CCCCCC)CCCCCCCC] | |
| 2-[(polyethylene glycol)-2000]-N,N-ditetradecylacetamide | 1.5-2.0 (e.g., 1.8) |
| (also known as 2-[2-(ω-methoxy (polyethyleneglycol2000) | |
| ethoxy]-N,N-ditetradecylacetamide) | |
| 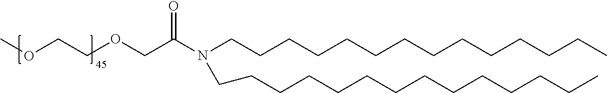 | |
| DSPC | 9.5-10.5 (e.g., 10) |
| Cholesterol, synthetic | 39-42 (e.g., 40.7) |

In some embodiments, an exemplary LNP formulation comprises an ionizable aminolipid, DSPC, cholesterol, and PEG-conjugated lipid at a molar ratio of approximately 50:10:38.5:1.5 or 47.5:10:40.8:1.7. In some embodiments, an ionizable amino lipid is or comprises ((4-hydroxybutyl)azanediyl)bis(hexane-6,1-diyl)bis(2-hexyldecanoate) (also known as 6-[N-6-(2-hexyldecanoyloxy)hexyl-N-(4-hydroxybutyl)amino]hexyl 2-hexyldecanoate).

In some embodiments, an exemplary LNP formulation comprises (i) SM-102 (heptadecan-9-yl 8 ((2 hydroxyethyl)(6 oxo 6-(undecyloxy)hexyl)amino)octanoate) within a range of 45 to 55 mol percent of total lipids; (ii) DSPC within a range of 5 to 15 mol percent of total lipids; (iii) cholesterol within a range of 35 to 45 mol percent of total lipids; and (iv) PEG2000-DMG within a range of 1 to 2 mol percent of total lipids; and RNA molecules as described herein that are encapsulated within or associated with the lipid nanoparticles.

In some embodiments, an exemplary LNP formulation comprises (i) ((4-hydroxybutyl)azanediyl)bis(hexane-6,1-diyl)bis(2-hexyldecanoate) (also known as 6-[N-6-(2-hexyldecanoyloxy)hexyl-N-(4-hydroxybutyl)amino]hexyl 2-hexyldecanoate) within a range of 45 to 55 mol percent of total lipids; (ii) DSPC within a range of 5 to 15 mol percent of total lipids; (iii) cholesterol within a range of 35 to 45 mol percent of total lipids; and (iv) a PEG-conjugated lipid within a range of 1 to 2 mol percent of total lipids; and RNA molecules as described herein that are encapsulated within or associated with the lipid nanoparticles.

### Example 5: Exemplary Prediction and/or Characterization of MHC Presentation

The present Example describes an exemplary approach to assessing MHC presentation which may be used in accordance with the present disclosure to select and/or characterize antigenic peptides as described herein.

In some embodiments, an antigenic peptide is selected and/or characterized through analysis of its amino acid sequence using an MHC-peptide presentation prediction algorithm or MHC-peptide presentation predictor, for example implemented in a computer processor (e.g., a computer processor that has been trained by a machine learning software), which determines a likelihood of binding and presentation of an epitope by an MHC class I or an MHC class II antigen.

In some embodiments, an MHC-peptide presentation prediction algorithm or MHC-peptide presentation predictor is or comprises neonmhc 1 and/or neonmhc 2, which predict and/or characterize likelihood of MHC class I and MHC class II binding, respectively. Alternatively or additionally, in some embodiments, an MHC-peptide presentation prediction algorithm or MHC-peptide presentation predictor is or comprises NetMHCpan or NetMHCIIpan. In some embodiments, a hidden markov model approach may be utilized for MHC-peptide presentation prediction and/or characterization. In some embodiments, the peptide prediction model MARIA may be utilized. In some embodiments, NetMHCpan is not utilized to predict or characterize likelihood of MHC binding for peptides as described herein. In some embodiments, the peptide prediction model MARIA may be utilized. In some embodiments, NetMHCIIpan is not utilized to predict or characterize likelihood of MHC binding for peptides as described herein. In some embodiments, neither NetMHCpan nor NetMHCIIpan is utilized to predict or characterize likelihood of MHC binding for peptides as described herein. In some embodiments, an MHC-peptide presentation prediction algorithm or MHC-peptide presentation predictor is or comprises RECON, which offers high quality MHC-peptide presentation prediction based on expression, processing and binding capabilities.

In some embodiments, multiple MHC-peptide presentation prediction algorithms or MHC-peptide presentation predictors may be utilized; in some such embodiments, results obtained with different strategies are compared with one another. In some embodiments, a determination that a particular peptide is likely to be or is significantly likely to be presented by MHC class I or MHC class II may be considered to be better established if two or more algorithms or predictors agree.

Alternatively or additionally, identification and/or characterization of MHC binding (e.g., of MHC class I and/or MHC class II binding) may involve experimental assessment, or reports thereof, which may involve presentation in one or more in vitro systems and/or in one or more organisms. In some embodiments, such assessment utilizes mammalian cells or systems; in some embodiments such assessment utilizes primate (e.g., in some embodiments, human and/or in some embodiments, non-human primate) cells or systems.

### Example 6: Exemplary HLA Class I and Class II Binding Assays

The present Example describes exemplary techniques for assessing peptide binding to HLA molecules. In some embodiments, exemplified technologies may determine and/or characterize (e.g., quantify) binding affinities for HLA class I and HLA class II.

In general, binding assays can be performed with peptides that are either motif-bearing or not motif-bearing. A detailed description of an exemplary protocol that can be utilized to measure the binding of peptides to Class I and Class II MHC has been published (Sette et al., Mol. Immunol. 31:813, 1994; Sidney et al., in Current Protocols in Immunology, Margulies, Ed., John Wiley & Sons, New York, Section 18.3, 1998). Briefly, purified MHC molecules (5 to 500 nM) are incubated with various unlabeled peptide inhibitors and 1-10 nM¹²⁵1-radiolabeled probe peptides for 48h in PBS containing 0.05% Nonidet P40 (NP40) (or 20% w/v digitonin for H-2 IA assays) in the presence of a protease inhibitor cocktail. Assays are typically performed at pH 7.0, though in some embodiments a lower pH (typically above about pH 4.0) may be performed.

Following incubation, MHC-peptide complexes are separated from free peptide, for example by gel filtration, e.g., on 7.8 mm×15 cm TSK200 columns (TosoHaas 16215, Montgomeryville, PA), though those skilled in the art will appreciate that column size can be adjusted, if desired, for example to improve separation of bound vs unbound peptides of a particular size or characteristic. The eluate from the TSK columns is passed through a Beckman 170 radioisotope detector, and radioactivity is plotted and integrated using a Hewlett-Packard 3396A integrator, and the fraction of peptide bound is determined.

Radiolabeled peptides can be iodinated using the chloramine-T method. Typically, in preliminary experiments, each MHC preparation is titered in the presence of fixed amounts of radiolabeled peptides to determine the concentration of HLA molecules necessary to bind 10-20% of the total radioactivity. Subsequent inhibition and direct binding assays can be performed using these HLA concentrations.

Since under these conditions [label]<[HLA] and IC₅₀>[HLA], the measured IC₅₀ values are often reasonable approximations of the true K_{D} values. Peptide inhibitors are typically tested at concentrations ranging from 120 μg/ml to 1.2 ng/ml, and are tested in two to four completely independent experiments. To allow comparison of the data obtained in different experiments, a relative binding figure is typically calculated for each peptide by dividing the IC₅₀ of a positive control for inhibition by the IC₅₀ for each tested peptide (typically unlabeled versions of the radiolabeled probe peptide). For database purposes, and inter-experiment comparisons, relative binding values can be compiled. Such values can subsequently be converted back into IC₅₀ nM values, for example by dividing the IC₅₀ nM of the positive controls for inhibition by the relative binding of the peptide of interest. This method of data compilation has proven to provide accurate and consistent comparison for peptides that have been tested on different days, or with different lots of purified MHC.

Alternatively or additionally, live cell/flow cytometry-based assays can also be used. This is a well-established assay utilizing the TAP-deficient hybridoma cell line T2 (American Type Culture Collection (ATCC Accession No. CRL-1992), Manassas, Va.). The TAP deficiency in this cell line leads to inefficient loading of MHCI in the ER and an excess of empty MHCIs. Salter and Cresswell, EMBO J. 5:94349 (1986); Salter, Immunogenetics 21:235-46 (1985). Empty MHCIs are highly unstable, and are therefore short-lived. When T2 cells are cultured at reduced temperatures, empty MHCIs appear transiently on the cell surface, where they can be stabilized by the exogenous addition of MHCI-binding peptides. To perform this binding assay, peptide-receptive MHCIs are induced by culturing aliquots of 10⁷ T2 cells overnight at 26° C. in serum free AIM-V medium alone, or in medium containing escalating concentrations (0.1 to 100 μM) of peptide. Cells are then washed twice with PBS, and subsequently incubated with a fluorescent tagged HLA-A02:01-specific monoclonal antibody, BB7.2, to quantify cell surface expression. Samples are acquired on a FACS Calibur instrument (Becton Dickinson) and the mean fluorescence intensity (MFI) determined using the accompanying Cellquest software.

### Example 7: Confirmation of Immunogenicity

The present Example describes an exemplary method for confirmation of immunogenicity, in particular by utilizing in vitro expansion (IVE) assays to test the ability of one or more antigens or peptides to expand CD8+ T cells.

Mature professional APCs are prepared for these assays in the following way. 80-90×10⁶ PBMCs from a healthy human donor are plated in 20 ml of RPMI media containing 2% human AB serum, and incubated at 37° C. for 2 hours to allow for plastic adherence by monocytes. Non-adherent cells are removed and the adherent cells are cultured in RPMI, 2% human AB serum, 800 IU/ml of GM-CSF and 500 IU/ml of IL-4. After 6 days, TNF-alpha is added to a final concentration of 10 ng/ml. On day 7, the dendritic cells (DC) are matured either by the addition of 12.5 mg/ml poly I:C or 0.3 μg/ml of CD40L. The mature dendritic cells (mDC) are harvested on day 8, washed, and either used directly or cryopreserved for future use.

For the IVE of CD8+ T cells, aliquots of 2×10⁵ mDCs are pulsed with each peptide at a final concentration of 100 micromole, incubated for 4 hours at 37° C., and then irradiated (2500 rads). The peptide-pulsed mDCs are washed twice in RPMI containing 2% human AB serum. 2×10⁵ mDCs and 2×10⁶ autologous CD8+ cells are plated per well of a 24-well plate in 2 ml of RPMI containing 2% human AB, 20 ng/ml IL-7 and 100 μg/ml of IL-12, and incubated for 12 days. The CD8+ T cells are then re-stimulated with peptide-pulsed, irradiated mDCs. Two to three days later, 20 IU/ml IL-2 and 20 ng/IL7 are added. Expanding CD8+ T cells are re-stimulated every 8-10 days, and are maintained in media containing IL-2 and IL-7. Cultures are monitored for peptide-specific T cells using a combination of functional assays and/or tetramer staining. Parallel IVES with the modified and parent peptides allowed for comparisons of the relative efficiency with which the peptides expanded peptide-specific T cells.

### Example 8: Quantitative and Functional Assessment of CD8+ and CD4+ T Cells

### Tetramer Staining

MHC tetramers are purchased or manufactured on-site, and are used to measure peptide-specific T cell expansion in the IVE assays. For the assessment, tetramer is added to 1×10⁵ cells in PBS containing 1% FCS and 0.1% sodium azide (FACS buffer) according to manufacturer's instructions. Cells are incubated in the dark for 20 minutes at room temperature. Antibodies specific for T cell markers, such as CD8, are then added to a final concentration suggested by the manufacturer, and the cells are incubated in the dark at 4° C. for 20 minutes. Cells are washed with cold FACS buffer and resuspended in buffer containing 1% formaldehyde. Cells are acquired on a FACS Calibur (Becton Dickinson) instrument, and are analyzed by use of Cellquest software (Becton Dickinson). For analysis of tetramer positive cells, the lymphocyte gate is taken from the forward and side-scatter plots. Data are reported as the percentage of cells that are CD8+/Tetramer+.

CD4⁺ T cell responses towards antigens or peptides can be tested using the ex vivo induction protocol. In this example, CD4+ T cell responses are identified by monitoring IFNγ and/or TNFα production in an antigen specific manner.

### Evaluation of Antigen Presentation:

For a subset of antigens or peptides (e.g., that are or comprise predicted or selected epitope(s) as described herein), affinity for the indicated HLA alleles and/or stability with the HLA alleles can be determined.

An exemplary detailed description of a protocol that can be utilized to measure the binding affinity of peptides to Class I MHC has been published (Sette et al, Mol. Immunol. 31(11):813-22, 1994). In brief, MHCI complexes are prepared and bound to radiolabeled reference peptides. Peptides are incubated at varying concentrations with these complexes for 2 days, and the amount of remaining radiolabeled peptide bound to MHCI is measured using size exclusion gel-filtration. The lower the concentration of test peptide needed to displace the reference radiolabeled peptide demonstrates a stronger affinity of the peptide for MHCI. Peptides with affinities to MHCI <50 nM are generally considered strong binders while those with affinities <150 nM are considered intermediate binders and those <500 nM are considered weak binders (Fritsch et al, 2014).

An exemplary detailed description of a protocol that can be utilized to measure binding stability of peptides to Class I MHC has been published (Harndahl et al. J Immunol Methods. 374:5-12, 2011). Briefly, synthetic genes encoding biotinylated MIC-I heavy and light chains are expressed in E. coli and purified from inclusion bodies using standard methods. The light chain (β2m) is radio-labeled with iodine (125I), and combined with the purified MHC-I heavy chain and peptide of interest at 18° C. to initiate pMHC-I complex formation. These reactions are carried out in streptavidin coated microplates to bind the biotinylated MHC-I heavy chains to the surface and allow measurement of radiolabeled light chain to monitor complex formation. Dissociation is initiated by addition of higher concentrations of unlabeled light-chain and incubation at 37° C. Stability is defined as the length of time in hours it takes for half of the complexes to dissociate, as measured by scintillation counts. MHC-II binding affinity with peptides is measured following the same general procedure as with measuring MHCI-peptide binding affinity. Prediction algorithms utilized for predicting MHCII alleles for binding to a given peptide are described herein. Alternatively or additionally, NetMHCIIpan may be utilized for prediction of binding.

To assess whether particular peptides or epitopes could be processed and presented from a larger polypeptide context, peptides eluted from HLA (class I or class II) molecules isolated from cells expressing the genes of interest can be analyzed by tandem mass spectrometry (MS/MS).

### ELISPOT

Peptide-specific T cells are functionally enumerated, for example, using the ELISPOT assay (BD Biosciences), which measures the release of IFNgamma from T cells on a single cell basis. Target cells (T2 or HLA-A0201 transfected C1Rs) are pulsed with 10 uM peptide for 1 hour at 37° C., and washed three times. 1×10⁵ peptide-pulsed targets are co-cultured in the ELISPOT plate wells with varying concentrations of T cells (5×10² to 2×10³) taken from the IVE culture. Plates are developed according to the manufacturer's protocol, and analyzed on an ELISPOT reader (Cellular Technology Ltd.) with accompanying software. Spots corresponding to the number of IFNgamma-producing T cells are reported as the absolute number of spots per number of T cells plated. T cells expanded on modified peptides are tested not only for their ability to recognize targets pulsed with the modified peptide, but also for their ability to recognize targets pulsed with the parent peptide.

### CD107 Staining

CD107a and b are expressed on the cell surface of CD8+ T cells following activation with cognate peptide. The lytic granules of T cells have a lipid bilayer that contains lysosomal-associated membrane glycoproteins (“LAMPs”), which include the molecules CD107a and b. Without wishing to be bound by any one theory, it is proposed that, when cytotoxic T cells are activated through the T cell receptor, the membranes of these lytic granules mobilize and fuse with the plasma membrane of the T cell. The granule contents are released, and this leads to the death of the target cell. As the granule membrane fuses with the plasma membrane, C107a and b are exposed on the cell surface, and therefore are markers of degranulation. Because degranulation as measured by CD107 a and b staining is reported on a single cell basis, the exemplary assay is used to functionally enumerate peptide-specific T cells. To perform the assay, peptide is added to HLA-A0201-transfected cells C1R to a final concentration of 20 μM, the cells are incubated for 1 hour at 37° C., and washed three times. 1×10⁵ of the peptide-pulsed C1R cells are aliquoted into tubes, and antibodies specific for CD107 a and b are added to a final concentration suggested by the manufacturer (Becton Dickinson). Antibodies are added prior to the addition of T cells in order to “capture” the CD107 molecules as they transiently appear on the surface during the course of the assay. 1×10⁵ T cells from the culture are added next, and the samples are incubated for 4 hours at 37° C. The T cells are further stained for additional cell surface molecules such as CD8 and acquired on a FACS Calibur instrument (Becton Dickinson). Data is analyzed using the accompanying Cellquest software, and results are reported as the percentage of CD8+CD107 a and b+ cells.

### CTL Lysis

Cytotoxic activity can be measured, for example, using a chromium release assay. Target T2 cells are labeled for 1 hour at 37° C. with Na⁵¹Cr and washed 5×10³ target T2 cells are then added to varying numbers of T cells from the IVE culture. Chromium release is measured in supernatant harvested after 4 hours of incubation at 37° C. The percentage of specific lysis is calculated as:

Experimental release−spontaneous release/Total release−spontaneous release×100.

### Example 9: Administration of Polyepitopic Compositions

The present Example describes exemplary administration of compositions that comprise or deliver a plurality of epitopes.

For example, a polyepitopic vaccine (e.g., that comprises or delivers a collection of epitopes—e.g., as individual discrete peptides or as one or more polyepitopic peptides such as one or more string constructs as described herein).

In some embodiments, a polyepitopic vaccine comprises or delivers multiple cytotoxic T lymphocyte (CTL) and/or helper T lymphocyte (HTL) epitopes. In some embodiments, such a vaccine is administered to subject(s) at risk of or having experienced exposure to infection.

In some embodiments, a polyepitopic vaccine as described herein comprises or delivers one or more polypeptides, each of which encompasses multiple epitope. In some embodiments, one or more monoepitopic peptides or polyepitopic antigens is delivered to a subject by administration of one or more a nucleic acid (e.g., DNA or and RNA) constructs. In some embodiments, a single nucleic acid construct (e.g., a DNA or RNA encoding a polyepitopic antigen) is administered. In some embodiments, a plurality of nucleic acids (e.g., each encoding a different monoepitopic or polyepitopic antigens) is administered. In some embodiments, an administered nucleic acid is a polyribonucleotide (e.g., an RNA, e.g., an mRNA); in some embodiments, a nucleic acid (e.g., an RNA) is administered in an LNP composition.

In some embodiments, an administered composition includes an aqueous carrier and/or alum.

In some embodiments, an initial administration is followed by one or more booster doses. In some embodiments, a booster dose includes the same amount of polyepitopic construct as the initial dose. In some embodiments, a booster dose include more or less of a polyepitopic construct than was provided in the initial dose. In some embodiments, a booster dose is administered after an interval of at least 1, 2, 3, 4, 5, 6, 7, 8, 9, 12, 11, 12, 13, 14, 15, 16, 17, 18, 29, 20, 21, 22, 23, 24 weeks or more after an initial dose. In some embodiments, multiple booster doses are administered. In some embodiments, each subsequent booster is administered at an interval that is the same as or longer than that between its immediate predecessor dose and the dose before that. In some embodiments, 2, 3, 4, 5, 6, or more boosters are administered. In some embodiments, not more than 4 doses total are administered. In some embodiments, not more than 3 doses total are administered. In some embodiments, not more than two doses total are administered. In some embodiments, not more than 1, 2, 3 or 4 doses are administered within a particular 12 month period. In some embodiments, not more than 3, not more than 2, or not more than 1 dose(s) is/are administered within a particular 12 month period (e.g., within 12 months of the initial dose).

In some embodiments, evaluation of an induced immune response (e.g., of magnitude, character, and/or diversity of immune response, such as antibody and/or T cell response) is performed before and/or after one or more doses (e.g., 1, 2, 3, 4, or more weeks after administration of a particular dose and/or within 6, 5, 4, 3, 2, or 1 month of administration of a particular dose) and may, for example be considered in determination of whether one or more booster doses should be administered and/or timing of such booster dose administration. In some embodiments, assessment of an immune response may utilize, for example, techniques that determine presence and/or level of epitope-specific CTL populations in a PBMC sample.

### Example 10: Administration of Dendritic Cells

The present Example describes exemplary dendritic cell compositions that comprise or deliver antigens as described herein.

In this example, peptides comprising epitopes as described herein (e.g., identified, designed, selected and/or characterized as described herein) are loaded onto dendritic cells. Peptide-pulsed dendritic cells can be administered to a subject. In some embodiments, such administration may stimulate a CTL response in vivo.

In this particular Example, dendritic cells (e.g., autologous dendritic cells) are isolated, expanded, and pulsed with peptide CTL and/or HTL epitopes as described herein. Dendritic cells may then be infused back into the patient. Such infusion can elicit CTL and/or HTL responses in vivo. The induced CTL and HTL then destroy (CTL) or facilitate destruction (HTL) of target cells (e.g., liver cells) that bear the proteins from which the epitopes in the vaccine are derived.

Ex vivo CTL or HTL responses to a particular antigen can be induced by incubating in tissue culture the patient's, or genetically compatible, CTL or HTL precursor cells together with a source of antigen-presenting cells, such as dendritic cells, and the appropriate immunogenic peptides.

After an appropriate incubation time (typically about 7-28 days), in which the precursor cells are activated and expanded into effector cells, the cells are infused back into the patient, where they will destroy (CTL) or facilitate destruction (HTL) of their specific target cells, i.e., cells displaying relevant epitopes.

### Example 11: Administration of Epitope Binding Agents

The present Example describes administration of epitope binding agents, as an alternative or complement to vaccination strategies describes herein.

For example, among other things, the present disclosure provides technologies for identification and/or characterization of monkeypox antigens that are particularly amenable to targeting in order to disrupt one or more features of infection. In many embodiments described herein, the present disclosure provides technologies that involve administration or delivery of antigens that are or comprise such epitopes, for example, in order to induce an immune response targeting such epitope(s) in a recipient.

Alternatively or additionally, the present disclosure provides and encompasses technologies for developing, characterizing, and/or administering agents that bind to such epitopes. In some embodiments, such strategies may provide or represent therapeutic interventions, for example useful in addition or as an alternative to vaccination strategies.

Epitope binding agents, such as antibody agents, TCR agents, CAR agents, and/or cells expressing any of the foregoing can be administered in accordance with methodologies known in the art and/or described herein.

In some embodiments, a relevant epitope binding agent can be delivered by administration of a composition that is or comprises the polypeptide binding agent. Alternatively or additionally, in some embodiments, a relevant epitope binding agent can be delivered by administration of a composition that is or comprises a polynucleotide (e.g., a DNA or RNA and, in many favored embodiments, an RNA) encoding the polypeptide binding agent. In some embodiments, a polypeptide binding agent is delivered by administered by a cell (or population thereof) that comprises or expresses the polypeptide and/or a polynucleotide that encodes it.

### Example 12: Exemplary Identification and/or Characterization of Variant Sequences with Immunogenic Potential

The present Example describes technologies for identification and/or characterization of peptide sequences that differ from a relevant reference, for assessment of immunogenic potential.

The full-length amino acid sequence of a variant protein (e.g., as observed in a circulating strain or developed through a predictive model) was derived.

Constituent 9-mer and 10-mer peptide fragments of the variant protein are each scored for binding potential on common HLA alleles (including, e.g., but not limited to HLA-A01:01, HLA-A02:01. HLA-A03:01, HLA-A24:02, HLA-B07:02, and HLA-B08:01) using available algorithms. Peptides scoring better than 1000 nM are noted as potential candidates.

Alternatively or additionally, constituent 9-mer or 10-mer peptide sequences not found in the reference protein sequence are flagged and scored for binding potential on common HLA alleles (including, e.g., but not limited to HLA-A01:01, HLA-A02:01. HLA-A03:01, HLA-A24:02, HLA-B07:02, and HLA-B08:01) using available algorithms.

### Example 13: Exemplary Monkeypox Peptide String Designs

The present Example exemplifies certain constructs (referred to herein as “strings”) of multiple monkeypox epitopes linked to one another and useful, for example, in vaccine compositions or otherwise as described herein.

Strings described in the present Example are designed to contain specific epitopes of monkeypox, each of which is disclosed herein and, e.g., is predicted and/or selected as described here, for example through use of an MHC-binding algorithm as described herein. The strings presented in the present Example are designed for therapeutic use in preventing and/or treating monkeypox and can be administered as polynucleotide constructs, e.g., mRNA encapsulated in a lipid nanoparticle.

In some embodiments, strings exemplified herein are encoded in an RNA that includes a 5′-UTR and 3′-UTR. Epitopes are interconnected by peptide linkers, encoded by their respective polynucleotide sequences. In some embodiments, one or more linkers may have a specific cleavage site.

Exemplary amino acid sequences for exemplary strings in Table 9 shows additional exemplary amino acid sequences for exemplary CD8 string and CD4 strings. In some embodiments, polynucleotide sequences are codon optimized (e.g., for efficient translation in humans).

### Example 14: Exemplary Antigen Identification, Selection and/or Characterization

Proteins expressed prior to cell infiltration/infection and include one or more portions expected or known to interface with host cytoplasm are identified, for example by literature review, considering transcriptomic (i.e., RNA expression levels) and/or proteomic (i.e., expressed protein levels) data. Degree of conservation of candidate proteins across relevant monkeypox strains (e.g., in relevant geographic region) is considered.

Various lab and field isolate strains can be considered for assessing conserved proteins and T cell epitopes. In some embodiments, strain Dumas was considered for assessing conserved proteins and T epitopes.

Immunogenicity of conserved proteins was also considered, for example by review of literature and/or application of predictive algorithms.

In some embodiments, an antigen may be or comprise one or more, and specifically may comprise a plurality, of distinct portions (e.g., epitope-containing fragments) of one or more of these proteins, for example in a string construct as described herein. In some embodiments, exemplary CD8 string candidates were designed utilizing one or more epitope-containing fragments of a plurality of antigens of Table 1 or Table 2, or fragments thereof. In various embodiments, a CD8 string construct can include sequences such as one or more of an SP domain (MKMRVMAPRTLILLLSGALALTETWAGS; SEQ ID NO: 269), GS-enriched linkers (e.g., GGSGGGGSGG (SEQ ID NO: 176); and/or GGSLGGGGSG (SEQ ID NO: 241)), and MITD domain (IVGIVAGLAVLAVVVIGAVVATVMCRRKSSGGKGGSYSQAASSDSAQGSDVSLTA; SEQ ID NO: 219).

In some embodiments, exemplary CD4 string candidates were designed utilizing one or more epitope-containing fragments of a plurality of antigens of Table 1 or Table 2, or fragments thereof. In various embodiments, a CD4 string construct can include sequences such as one or more of an SP domain (MKMRVMAPRTLILLLSGALALTETWAGS; SEQ ID NO: 269), GS-enriched linkers (e.g., GGSGGGGSGG (SEQ ID NO: 176); and/or GGSLGGGGSG (SEQ ID NO: 241)), and MITD domain (IVGIVAGLAVLAVVVIGAVVATVMCRRKSSGGKGGSYSQAASSDSAQGSDVSLTA; SEQ ID NO: 219).

In some embodiments, a secretory signal (“Sec”) or a signal peptide (SP) domain present in exemplary string candidates described herein (e.g., a CD8 string and/or a CD4 string as described herein) may be that from HSV-2 gD SP MGRLTSGVGTAALLVVAVGLRVVCA (SEQ ID NO: 144); in alternative embodiments, a different secretory signal, e.g., from HSV-1 gD SP, is used. In some embodiments, a signal peptide may be or comprise an IgE signal peptide. In some embodiments, a signal peptide may be or comprise an IgE HC (Ig heavy chain epsilon −1) signal peptide. In some embodiments, a signal peptide that may be useful in accordance with the present disclosure may comprise one of the following sequences:

(SEQ ID NO: 242)
MDSKGSSQKGSRLLLLLVVSNLLLPQGVVG;
(SEQ ID NO: 150)
MDWTWILFLVAAATRVHS;
(SEQ ID NO: 149)
METPAQLLFLLLLWLPDTTG;
(Japanese encephalitis PRM signal sequence)
(SEQ ID NO: 151)
MLGSNSGQRVVFTILLLLVAPAYS;
(VSVg protein signal sequence)
(SEQ ID NO: 152)
MKCLLYLAFLFIGVNCA;
(Japanese encephalitis JEV signal sequence)
(SEQ ID NO: 243)
MWLVSLAIVTACAGA;
or
(SEQ ID NO: 154)
MFVFLVLLPLVSSQC.

In some embodiments, certain chunk boundary considerations are incorporated into the string constructs, for example establishing chunk boundaries to minimize presence of sequences (e.g., epitopes) that may overlap with the human proteome.

### Example 15: Exemplary Vaccine Composition

The present Example describes certain exemplary vaccine compositions.

In some embodiments, a provided vaccine candidate will contain at least 2 polyribonucleotides, at least one of which encodes a monkeypox antigen described herein (e.g., a full length monkeypox protein or one or more fragments or epitopes thereof, such as a string construct described herein), and optionally at least one of which encodes at least one other conserved monkeypox protein (or fragment(s) or epitope(s) thereof, such as in a string construct as described herein; in some embodiments such a string construct may include fragments or epitopes from two or more different monkeypox proteins).

In some embodiments, two or more (e.g., 3, for example 2 of which are/encode monkeypox antigen string constructs and one of which is/encodes a string construct of a plurality of CD8 and/or CD4 epitopes from other conserved monkeypox proteins) are formulated together in a single LNP formulation; in other embodiments, individual polyribonucleotides may be separately formulated in (the same or different) LNP formulations and such may be mixed together (e.g., in a 1:1 ratio of each polyribonucleotide, or alternatively in a 1:1 ratio of monkeypox-antigen-encoding-polyribonucleotide to “other” polyribonucleotide so that, for example, for a composition comprising 2 monkeypox-antigen-encoding polyribonucleotides and one other polyribonucleotide, the ratios would be 0.5:0.5:1).

### Example 16: Exemplary Pre-Clinical Assessment

The present Example describes certain pre-clinical assessments that may be performed of certain RNA vaccine compositions described herein:

In some embodiments, one vaccine candidate is assessed. In some embodiments, more than one different vaccine candidate may be assessed. In some such embodiments, different candidates may vary, for example, in:
RNA platform (e.g., unmodified RNA, modified RNA, saRNA);
Encoded antigen(s);
Number of RNAs;
Elements of RNA construct (e.g., cap and/or cap-adjacent sequences, 5′-UTR, 3′-UTR, and/or PolyA tail); and/or
Lipid composition of LNP.

In some embodiments, pre-clinical assessment of certain polyribonucleotide vaccine compositions (e.g., LNP formulated mRNA-based monkeypox vaccines) comprises one or more of assessment in challenge experiments, assessment of level of protection, assessment of immunogenicity, and/or assessment of functional antibody responses.

LNP formulated mRNA-based monkeypox vaccines are tested in a challenge model. Non-human primate models, such as Rhesus macaques and Cynomolgus monkey, and/or rodent models, such as C57/B16 mice, Balb/c mice or NODscidIL2Rγnull mice; and/or guinea pig models, inoculated with monkeypox, are administered a first vaccination and can be administered an additional vaccination (e.g., 1, 2, 3, 4, 5, 6, 7, 8, 9, or 10 additional vaccinations) following the first vaccination. Wherein more than one vaccination is administered, the vaccinations are administered at an interval of 1, 2, 3, 4, 5, 6, 7, or 8 week intervals). Following vaccination, animals are challenged by monkeypox. Alternatively or additionally, animals are challenged by intravenous, subcutaneous, and/or intramuscular injection of virus-infected lymphocytes. Lymphocytes can be infected with any suitable strain of monkeypox. Animals are then evaluated for reduced infection of neurons. In some embodiments, a non-human primate and/or mouse is challenged in a plurality of instances (e.g., before first vaccination and/or wherein additional vaccinations are administered, at any time point between or after vaccinations). Following challenge, animals subjected to the study may be assessed according to any method known in the art, including, for example, serology assessment, immunogenicity, level of protection, etc.

In some embodiments, serum antibody characterization and/or serum transfer experiments (e.g., from one vaccinated species to a different non-vaccinated species, e.g., from vaccinated non-human primate to non-vaccinated mouse) are conducted (e.g., to assess protective antibody response).

In some embodiments, certain polyribonucleotide vaccine compositions of the present disclosure are assessed for level of protection. Level of protection can be assessed according to any suitable method known in the art.

In some embodiments, certain polyribonucleotide vaccine compositions of the present disclosure are assessed for immunogenicity. For example, ELISA can be used to determine IgG specific (and subclasses thereof) titers and/or avidity of antibodies generated in response to certain polyribonucleotide vaccine compositions of the present disclosure to monkeypox antigens. In some embodiments, serum antibody titers against monkeypox glycoprotein (e.g., gE glycoprotein, etc.) is determined by ELISA using standard methods. In some embodiments, for example, ELISpot (e.g., for CD8+, CD4+ T cells and/or IFNγ) and assessment of pro-inflammatory cytokine responses with splenocytes from immunized and/or challenged animal models and peptide pools derived from vaccine targets can also be assessed. In some embodiments, for example, phenotyping of immune responses (e.g., by flow cytometry) are assessed. In some embodiments, for example, T cell depletion and/or protection assays are conducted to assess immunogenicity (e.g., according to any suitable known method in the art).

In some embodiments, one or more functional responses of antibodies generated in response to certain polyribonucleotide vaccine compositions of the present disclosure are assessed. Functional antibody responses can be assessed, for example, using a monkeypox neutralization assay. In some embodiments, a monkeypox in vitro neutralization assay is performed to evaluate anti-monkeypox glycoprotein (e.g., gE) antibodies in neutralizing monkeypox. For example, anti-monkeypox glycoprotein antibodies are obtained by collecting the sera of animals (e.g., mice) vaccinated with monkeypox mRNA vaccines. monkeypox virus are added to the diluted sera and neutralization is allowed to continue for 1 hour at room temperature. ARPE-19 cells are seeded in 96-wells one day before and the virus/serum mixtures are added to ARPE-19 cells at 50-100 pfu per well. The ARPE-19 cells are fixed on the next day and monkeypox-specific staining is performed. The plates are scanned and analyzed. A neutralization titer is expressed as the highest serum dilution required to achieve a 50% reduction in the number of plaques.

In some embodiments, functional antibody responses can be assessed, for example, using passive transfer studies of sera from immunized animals to naïve animals that are challenged and assessing level of protection.

### Example 17: Exemplary Characterization Studies

The present Example describes certain potential characterization studies that may be utilized, for example, to identify, select, and/or characterize vaccine candidates or vaccine compositions (e.g., manufacturing batches thereof), or components thereof as described herein.

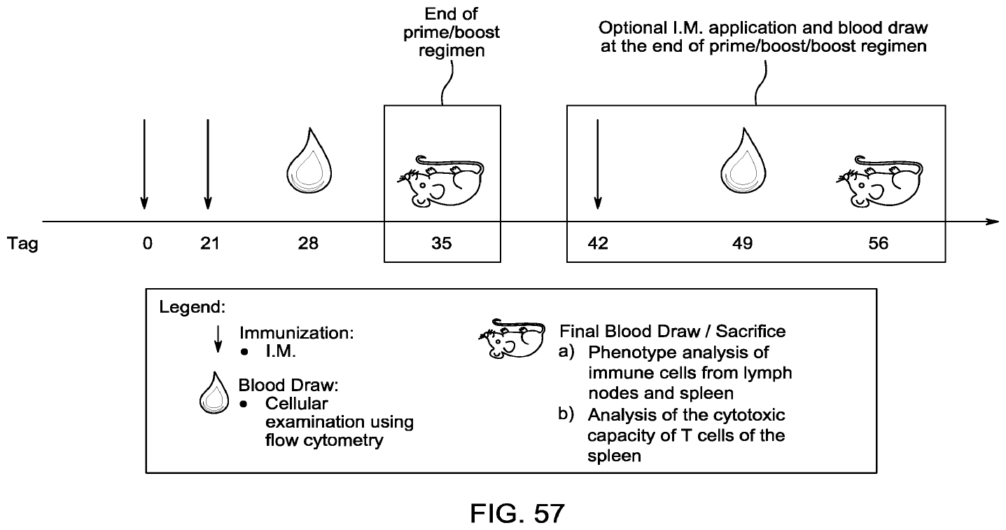

FIG. 57 presents a potential immunization protocol that can be utilized to assess ability of a vaccine candidate that comprises or delivers an antigen(s) as described herein to induce B- and/or T-cells, e.g., after intramuscular immunization, directed to the antigen(s) and/or epitope(s) thereof. In some embodiments, level and/or diversity of response is determined. In some embodiments, presence and/or level of neutralizing antibodies is/are determined. In some embodiments, protection of the immunized subject from challenge with monkeypox is assessed.

Alternatively or additionally, in some embodiments, one or more in vitro assessments may be performed, for example:
(1) in vitro expression of an antigen encoded by a polyribonucleotide included in a vaccine composition; and/or
(2) in vitro potency of antigen expressed from a polyribonucleotide included in a vaccine composition as described herein.

### Example 18: Exemplary Clinical Studies of Polyribonucleotide Vaccine Compositions

The present Example describes certain clinical assessments that may be performed of certain polyribonucleotide vaccine compositions described herein.

In some embodiments, more than one different vaccine candidate may be assessed. In some such embodiments, different candidates may vary, for example in:
1. RNA platform (e.g., unmodified RNA, nucleoside-modified RNA, self-amplifying RNA (saRNA), trans-amplifying RNA);
2. encoded antigen—e.g.,
   i. which monkeypox protein(s) utilized
   ii. full length protein antigen vs fragment vs plurality of fragments vs fusion with one or more heterologous sequences (e.g., membrane tether, secretion, linker(s))
   iii. epitopes from different (and/or multiple) phases of monkeypox life cycle
3. number of RNAs
4. elements of RNA construct
   i. cap and/or cap-adjacent sequences
   ii. 5′ UTR
   iii. 3′ UTR
   iv. polyA tail
5. lipid composition of LNP.

In one particular embodiment, up to three candidate vaccines that include polyribonucleotides having a first sequence that encodes at least one antigen of Table 1 or fragments thereof, and further include polyribonucleotides having a second sequence that encodes at least one epitope that is a fragment of an antigen of Table 2. In one particular embodiment, candidate vaccines that include a plurality of polyribonucleotides having distinct sequences that each respectively encode at least one antigen of Table 1 or fragments thereof, and further include polyribonucleotides having a sequence that encodes a plurality of epitopes that are each a fragment of an antigen of Table 2. In this particular exemplary embodiment, vaccine candidates may be evaluated by intramuscular administration, for example, based on a dose-escalation scheme.

### Example 19: Exemplary Production, Characterization, and or Use of Certain Polyepitopic Vaccine Compositions

In some embodiments, immunogenicity of a multi-epitopic polyribonucleotide or polypeptide is tested in rodents (e.g., mice, e.g., transgenic mice) to evaluate the magnitude of immune response induced against the epitopes tested. In some embodiments, immunogenicity of encoded epitopes in vivo can be correlated with in vitro responses of specific CTL lines against target cells expressing the multi-epitope polypeptides. Thus, in some embodiments, such exemplary experiments can show that a multi-epitopic construct serves to both: 1) generate a cell mediated and/or humoral response and 2) induce immune cells to recognize cells expressing the encoded epitopes.

In some embodiments, for example, to create a DNA sequence encoding the selected multi-epitope construct (e.g., DNA or RNA) for expression in human cells, amino acid sequences of epitopes to be included can be reverse translated. A human codon usage table can be used to guide the codon choice for each amino acid.

In some embodiments, epitope-encoding DNA sequences are directly adjoined, so that when transcribed and translated, a continuous polypeptide sequence is created.

In some embodiments, expression and/or immunogenicity is optimized. In some such embodiments, expression and/or immunogenicity is optimized by incorporating additional elements into an encoding construct. Without limitation, examples of amino acid sequences that can be reverse translated and included in a multi-epitopic construct sequence include, for example: HLA class I epitopes, HLA class II epitopes, a ubiquitination signal sequence, and/or an endoplasmic reticulum targeting signal. In some embodiments, HLA presentation of CTL and HTL epitopes can be improved by including synthetic (e.g., poly-alanine) or naturally-occurring flanking sequences adjacent to the CTL or HTL epitopes; larger peptides comprising the epitope(s) are within the scope of the present disclosure.

In some embodiments, a multi-epitope-encoding DNA sequence can be produced by assembling oligonucleotides that encode the plus and minus strands of the construct. In some embodiments, overlapping oligonucleotides (e.g., 30-100 bases long) can be synthesized, phosphorylated, purified and annealed under appropriate conditions using a suitable technique known in the art. In some embodiments, ends of utilized oligonucleotides can be joined, for example, using ligation (e.g., T4 DNA ligation). In some embodiments, synthetic constructs encoding multiple epitopes can then be cloned into a desired expression vector (e.g., using a suitable cloning technique known in the art).

In some embodiments, standard regulatory sequences well known to those of skill in the art (e.g., promoters, enhancers, etc.) can be included to ensure expression of a polyepitopic construct in target cells. In some embodiments, for example, a promoter with a down-stream cloning site for insertion of the polyepitopic construct coding sequence; a polyadenylation signal for efficient transcription termination; an E. coli origin of replication; and an E. coli selectable marker (e.g., ampicillin or kanamycin resistance). In some embodiments, a utilized promoter or promoters is not limiting and various other suitable promoter sequences can be used for this purpose, e.g., the human cytomegalovirus (hCMV) promoter. See, e.g., U.S. Pat. Nos. 5,580,859 and 5,589,466 for other suitable promoter sequences.

In some embodiments, vector modifications are used to optimize expression and/or immunogenicity. In some embodiments, introns are utilized for efficient gene expression, and one or more synthetic or naturally-occurring introns are incorporated into the transcribed region. In some embodiments, inclusion of stabilization sequences (e.g., mRNA stabilization sequences) and/or sequences for replication in mammalian cells are used for increasing expression.

In some embodiments, once an expression vector is selected, a multi-epitopic construct coding sequence is cloned into a polylinker region downstream of a promoter (e.g., generating a “plasmid”). In some embodiments, such a plasmid is transformed into an appropriate E. coli strain, and DNA is prepared using any suitable technique known in the art. In some embodiments, the orientation and DNA sequence of the multi-epitopic encoding sequence, as well as all other elements included in the vector, are confirmed using, for example, restriction mapping and/or DNA sequence analysis. In some embodiments, bacterial cells comprising a desired plasmid can be stored, for example, as a master cell bank and/or a working cell bank.

In some embodiments, immunomodulatory sequences contribute to the immunogenicity, e.g., of nucleic acid vaccine constructs. In some embodiments, such sequences are included in a vector, outside the coding sequence, if desired to enhance immunogenicity. In some embodiments, such sequences are immunostimulatory. In some embodiments, such sequences are ISSs or CpGs.

In some embodiments, a bi-cistronic expression vector which allows production of both multi-epitopic construct and a second protein (e.g., included to enhance or decrease immunogenicity) are used. Without limitation, examples of proteins or polypeptides that can enhance the immune response if co-expressed with a multi-epitopic construct include cytokines (e.g., IL-2, IL-12, GM-CSF), cytokine-inducing molecules (e.g., LeIF), costimulatory molecules, or for HTL responses, pan-DR binding proteins. In some embodiments, helper (HTL) epitopes are fused and/or linked to intracellular targeting signals and expressed separately from expressed CTL epitopes; this allows direction of the HTL epitopes to a cell compartment different than that of the CTL epitopes. If required, this could facilitate more efficient entry of HTL epitopes into the HLA class II pathway, thereby improving HTL induction. In contrast to HTL or CTL induction, specifically decreasing the immune response by co-expression of immunosuppressive molecules (e.g. TGF-β) can be beneficial in certain diseases.

In some embodiments, commercially-relevant quantities of plasmid DNA (e.g., for administration or for production of RNA and/or protein for administration) can be produced, for example, by fermentation in E. coli, followed by purification. In some embodiments, aliquots from a working cell bank are used to inoculate growth medium, and grown to a predetermined level (e.g., saturation) in flasks (e.g., shaker flasks) or a bioreactor according to well-known techniques. In some embodiments, plasmid DNA is purified using standard bioseparation technologies such as, for example, solid phase anion-exchange resins supplied by QIAGEN, Inc. (Valencia, California). In some embodiments, supercoiled DNA is separated from open circular and linear forms using gel electrophoresis or other suitable methods known in the art.

In some embodiments, purified plasmid DNA is prepared for injection into a subject using a variety of formulations. In some embodiments, lyophilized DNA is reconstituted in sterile phosphate-buffer saline (PBS). This approach, known as “naked DNA,” is currently being used for intramuscular (IM) administration in clinical trials. In some embodiments, to maximize the immunotherapeutic effects of polyepitopic vaccine compositions, an alternative method for formulating nucleic acids (e.g., purified plasmid DNA, in vitro transcribed RNA, etc) can be used. A variety of methods have been described, and new techniques can become available. In some embodiments, cationic lipids are used in the formulation (see, e.g., as described by WO 93/24640; Mannino & Gould-Fogerite, BioTechniques 6(7): 682 (1988); U.S. Pat. No. 5,279,833; WO 91/06309; and Felgner, et al., Proc. Nat'l Acad. Sci. USA 84:7413 (1987). In some embodiments, glycolipids, fusogenic liposomes, peptides and compounds referred to collectively as protective, interactive, non-condensing compounds (PINC) are complexed to purified plasmid DNA to influence variables such as stability, intramuscular dispersion, or trafficking to specific organs or cell types.

In some embodiments, a polynucleotide is introduced into cells by use of high-speed cell deformation. During high-speed deformation, cells are squeezed such that temporary disruptions occur in the cell membrane, thus allowing the nucleic acid to enter the cell. In some embodiments, polypeptides are produced from expression vectors, e.g., in a bacterial expression vector, for example, and the proteins can then be delivered to the cell.

In some embodiments, target cell sensitization is used as a functional assay for expression and HLA class I presentation of encoded CTL epitopes. For example, in some embodiments, a polynucleotide is introduced into a mammalian cell line that is suitable as a target for standard CTL chromium release assays. In some embodiments, a transfection method used is dependent on the final formulation. In some embodiments, electroporation is used, e.g., for “naked” polynucleotides (e.g., DNA). In some embodiments, wherein cationic lipids are utilized, direct in vitro transfection is utilized as a transfection method. In some embodiments, a plasmid expressing marker protein or polypeptide (e.g., green fluorescent protein (GFP)) is co-transfected to allow enrichment of transfected cells (e.g., using fluorescence activated cell sorting (FACS)). In some embodiments, cells are then chromium-51 (⁵¹-Cr) labeled and used as target cells for epitope-specific CTL lines; cytolysis, detected by ⁵¹Cr release, indicates both production of, and HLA presentation of encoded CTL epitopes. In some such embodiments, expression of HTL epitopes can be evaluated in an analogous manner using assays to assess HTL activity.

In some embodiments, in vivo immunogenicity is utilized for functional testing. In some embodiments, rodents (e.g., mice, e.g., transgenic mice expressing appropriate human HLA proteins) are immunized with a polyepitopic vaccine composition (e.g., comprising a DNA or RNA active agent). In some embodiments, dose and route of administration are formulation dependent (e.g., IM for DNA in PBS or LNP-formulated DNA or RNA, intraperitoneal (IP) for lipid-complexed DNA). In some embodiments, for example, twenty-one days after immunization, splenocytes are harvested and restimulated for 1 week in the presence of peptides encoding each epitope being tested. Thereafter, for CTL effector cells, assays are conducted for cytolysis of peptide-loaded, ⁵¹Cr-labeled target cells using standard techniques. Lysis of target cells that are sensitized by HLA loaded with peptide epitopes, corresponding to minigene-encoded epitopes, demonstrates vaccine function for in vivo induction of CTLs. Immunogenicity of HTL epitopes is evaluated in transgenic mice in an analogous manner.

### Example 20: In Vitro Expression of Exemplary Polyribonucleotide Constructs

The present Example describes in vitro expression of exemplary polyribonucleotide constructs encoding exemplary monkeypox antigens. Specifically, the present example describes expression of exemplary polyribonucleotide constructs encoding certain exemplary monkeypox B cell antigens in mammalian cells (e.g., HEK293T cells).

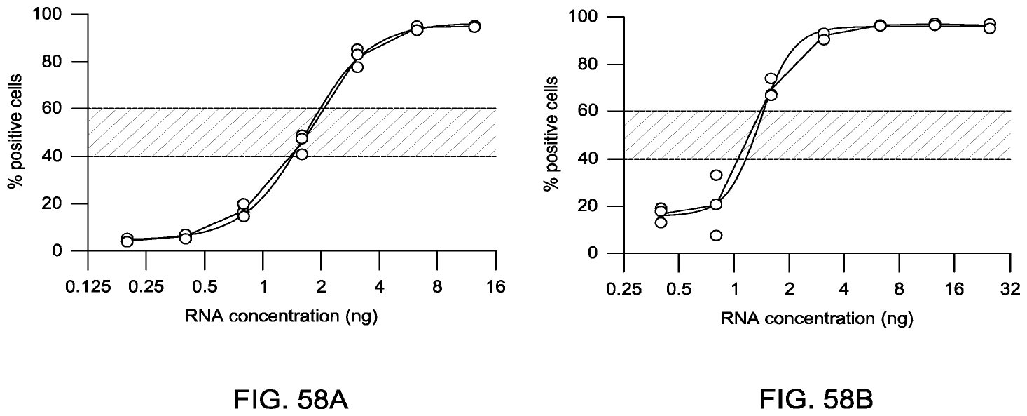

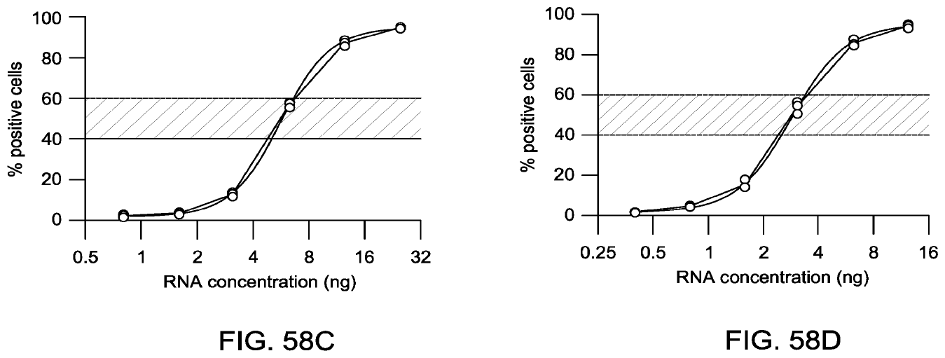

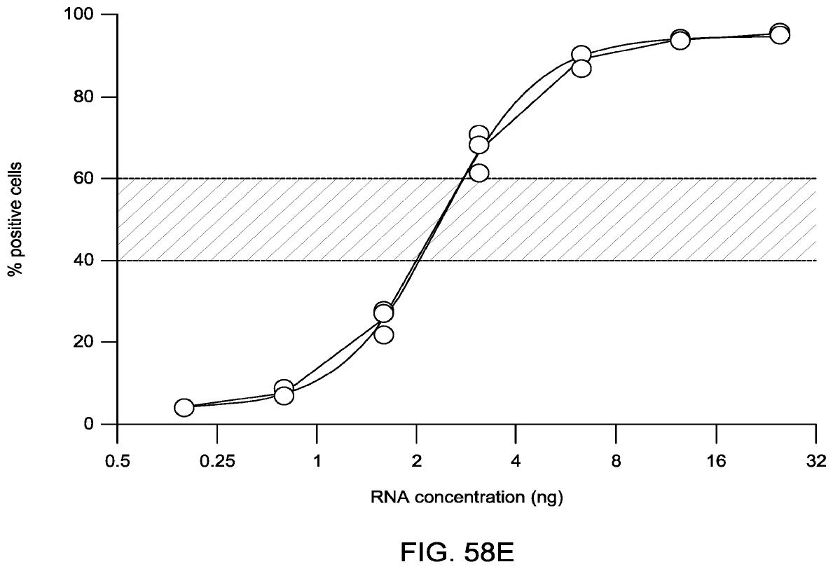

The transfection rate of exemplary polyribonucleotide constructs encoding CSP B cell antigens was also determined. Transfection rate indicates the percentage of HEK cells positive for presence of the protein. Specifically, HEK293T cells were transfected with varying concentrations of the different exemplary polyribonucleotide constructs and later harvested, permeabilized and stained with a viability dye and monoclonal antibodies staining respective antigens to allow quantification of total expression of the protein. Flow cytometry was used to quantify the number of transfected cells as well as protein expression. FIGS. 58A, 58B, 58C, 58D, and 58E are line graphs depicting transfection efficiency of exemplary polyribonucleotides encoding CSP antigens (E8, M1, H3, A35, and B6, respectively).

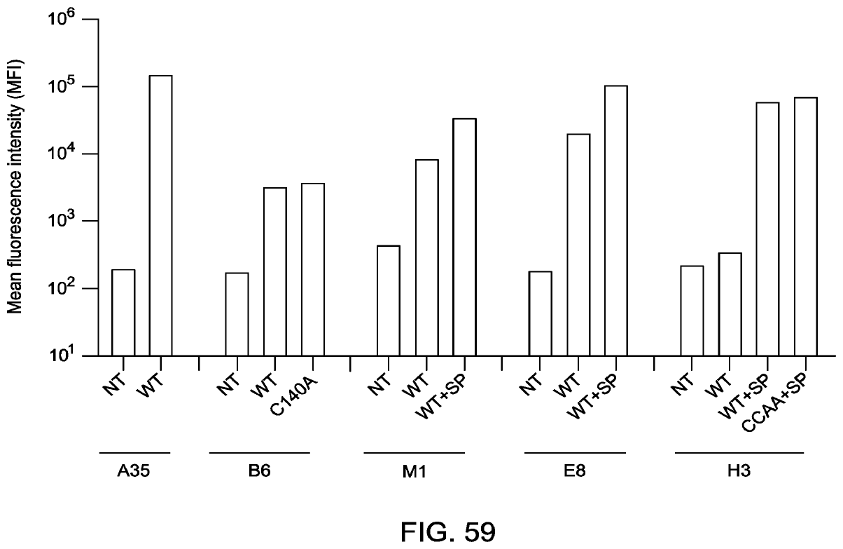

The expression rate of exemplary polyribonucleotide constructs encoding CSP B cell antigens was also determined. Expression rate represents the mean fluorescence of the total HEK population (both transfected and non-transfected cells). HEK293T cells were transfected with exemplary polyribonucleotide constructs encoding exemplary monkeypox B cell antigens (B6, A35, M1, E8 and H3) at a single concentration using RiboJuice. Following an overnight incubation surface expression of the target antigen was measured by flow cytometry. For A35, M1, E8 and H3 designs, expression was detected using mouse monoclonal antibodies raised to the corresponding MPXV protein. For B6, expression was detected using a polyclonal antibody against VACV version of the protein raised in rabbits. Primary antibody binding was detected using an anti-mouse or anti-rabbit IgG fluorophore conjugate. Results are depicted in FIG. 59. Heights of bars in FIG. 59 indicate the mean fluorescence intensity; data are the mean of 2 to 3 technical replicates. NT indicates non-transfected control for each primary antibody stain. WT refers to a wild-type antigen sequence, B6 C140A refers to a B6 antigen with a C140A substitution, +SP indicates the antigen includes a secretion signal (e.g., M1 WT+SP refers to an M1 antigen having a secretion signal, e.g., SS+MIR as described in Table 9), H3 CCAA+SP refers to an H3 antigen having a secretion signal and C86A and C90A substitutions.

Thus, the present example confirms successful expression of various polyribonucleotide constructs encoding exemplary monkeypox CSP antigens HEK293T cells.

### Example 21: Exemplary Mouse Immunogenicity Studies

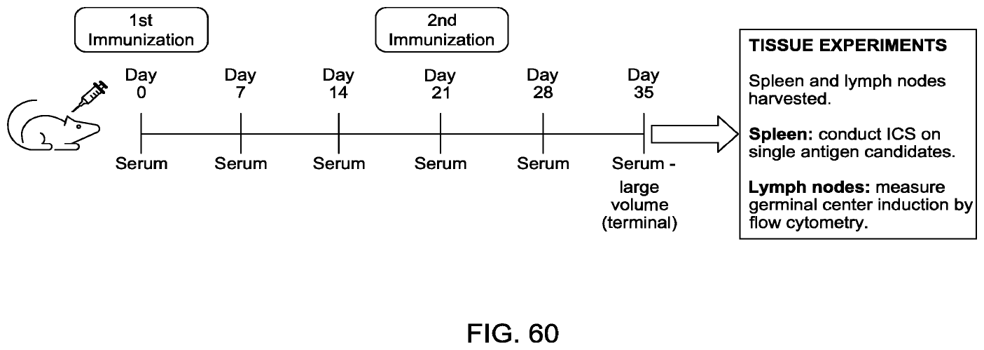

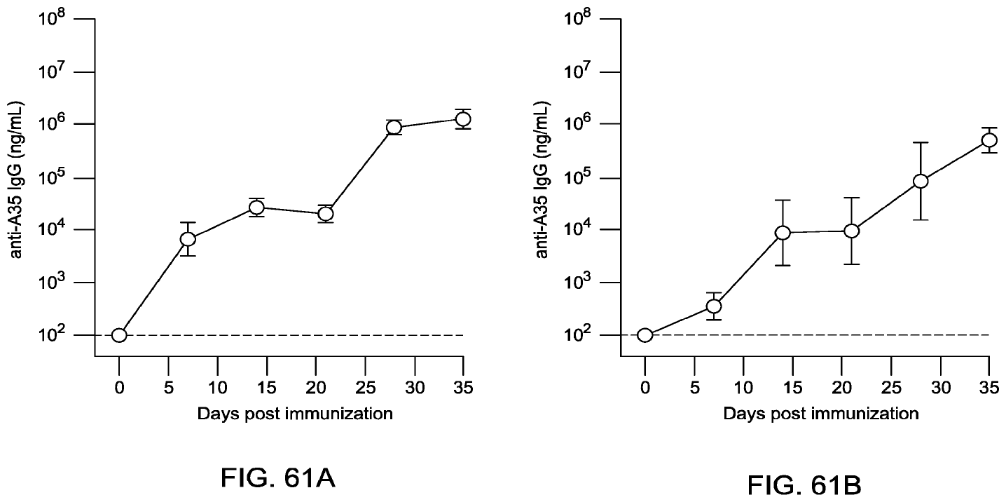

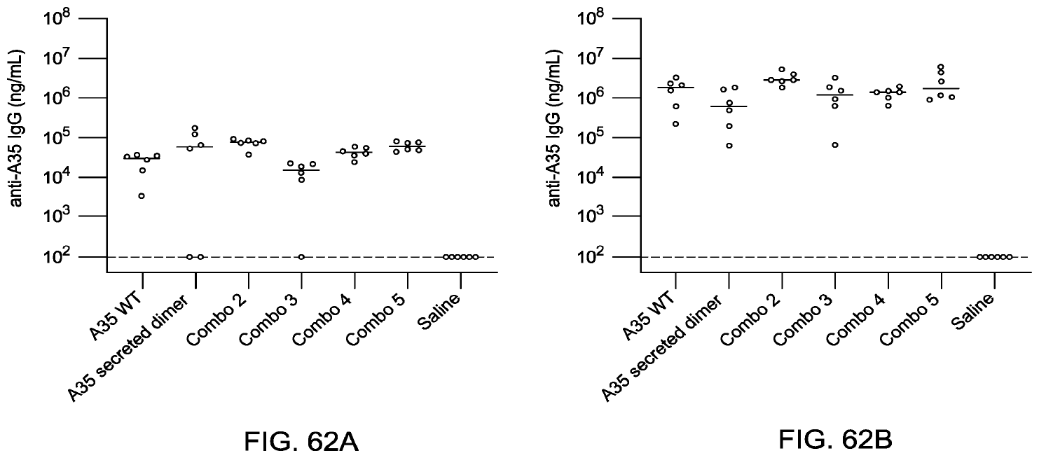

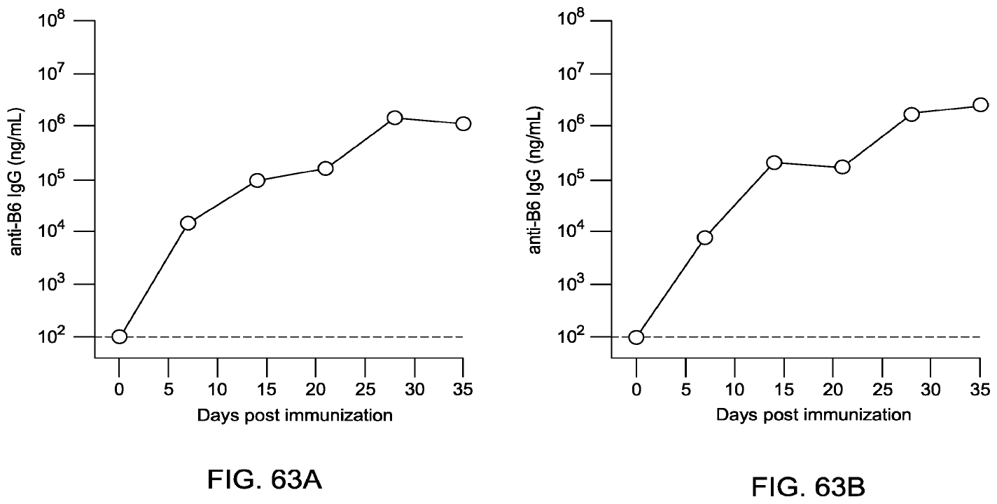

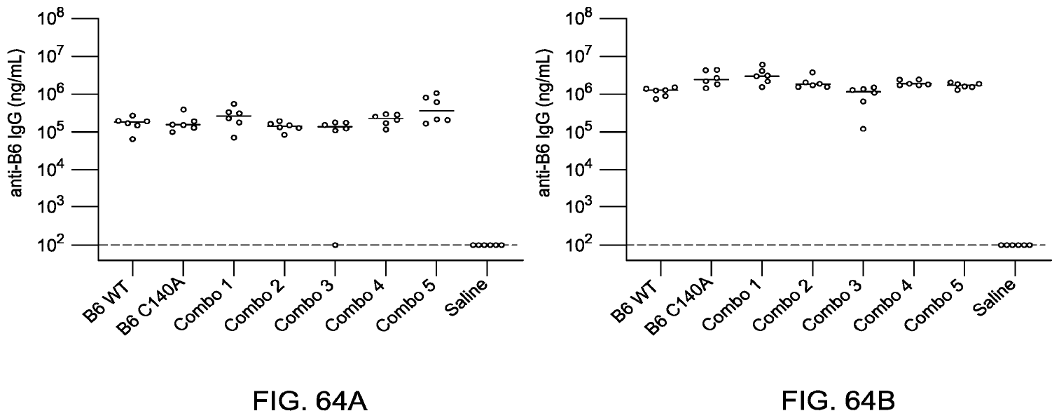

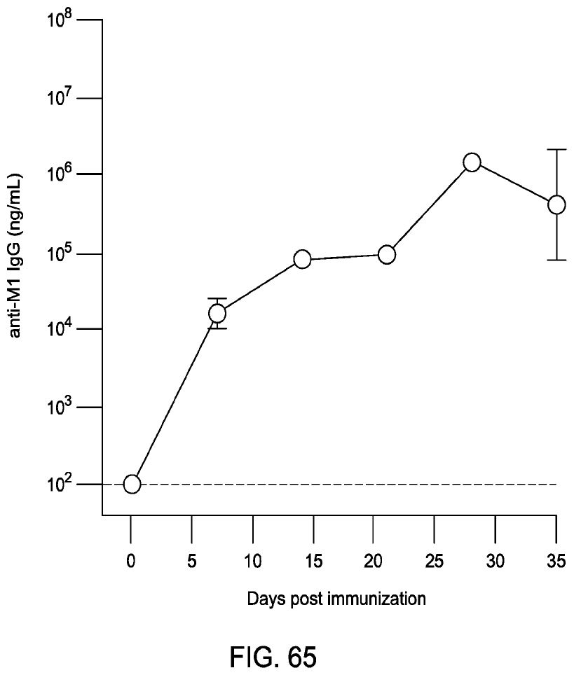

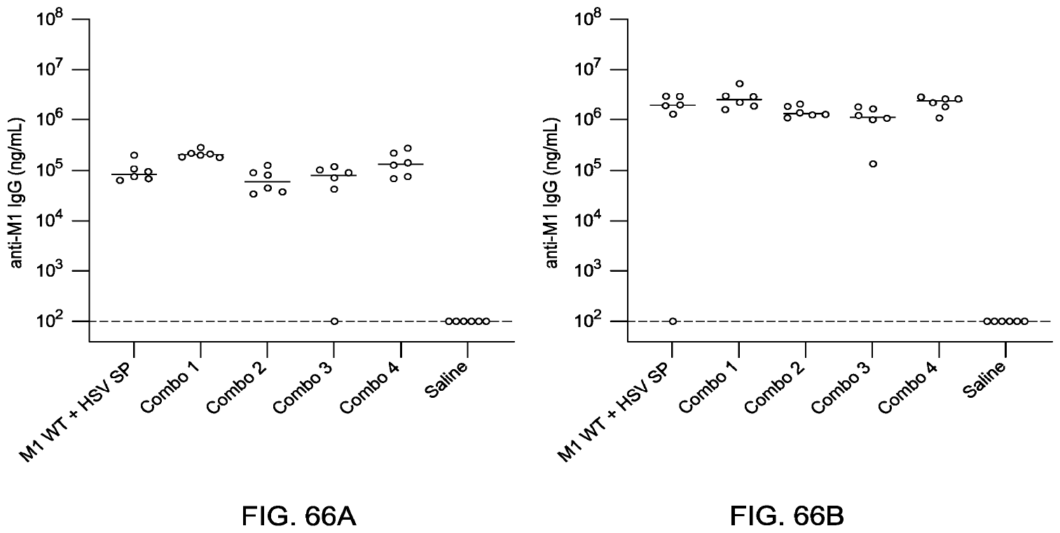

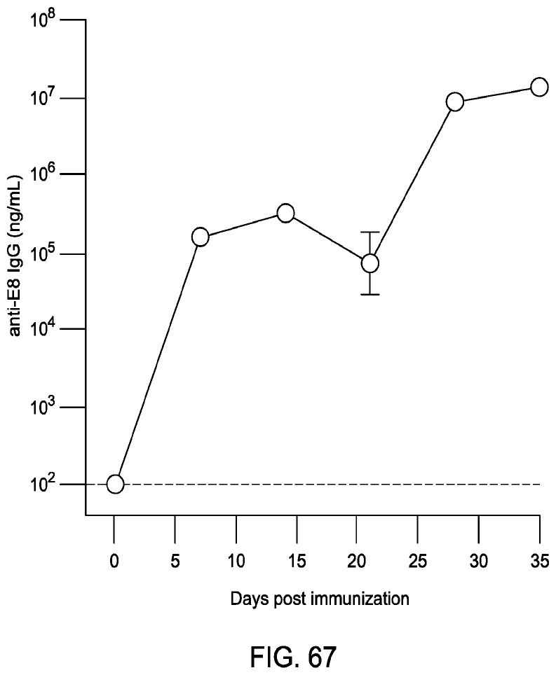

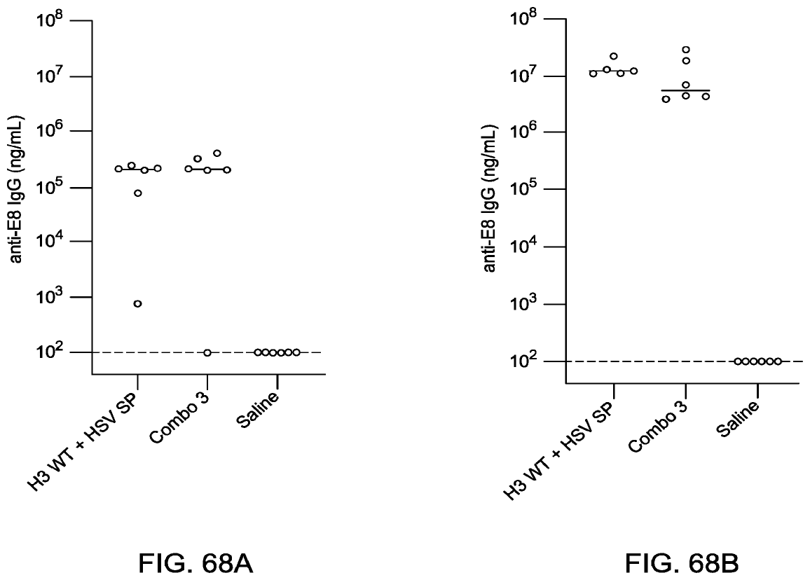

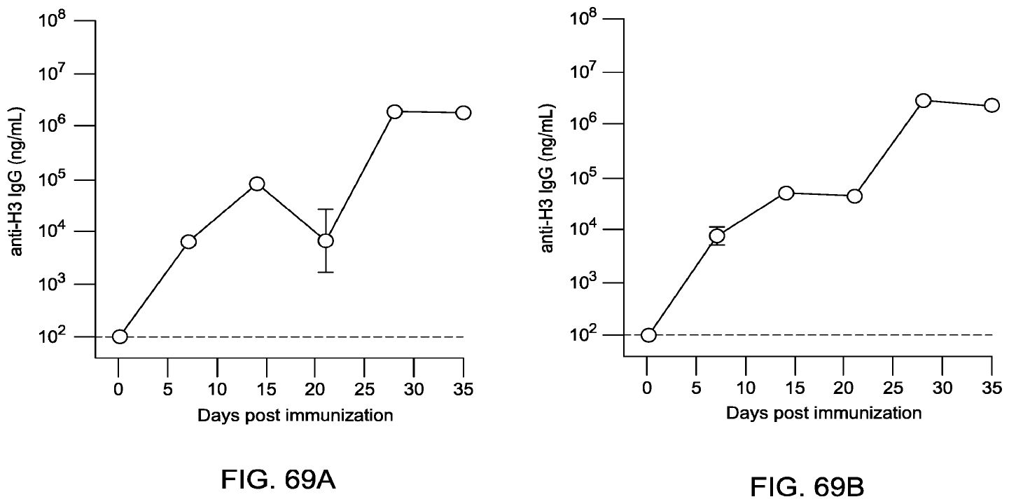

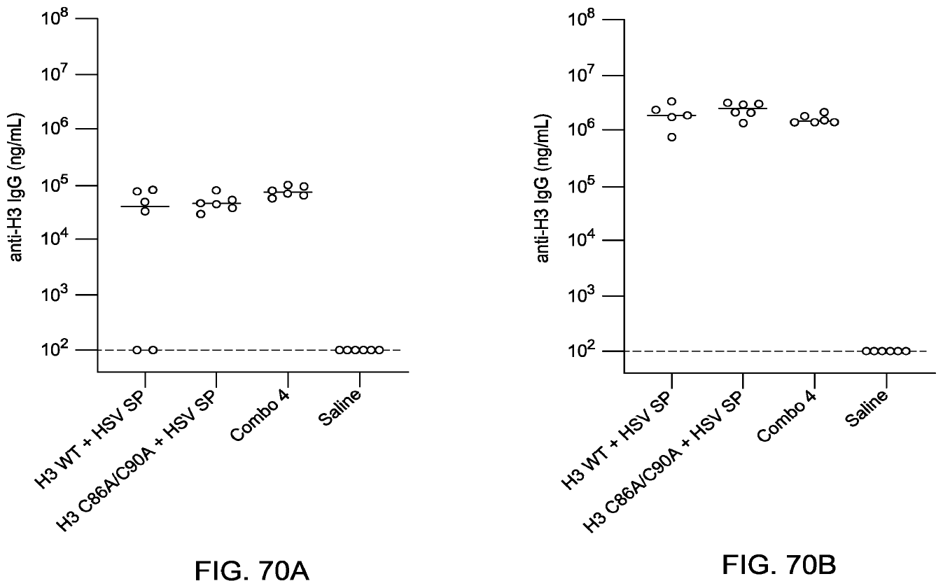

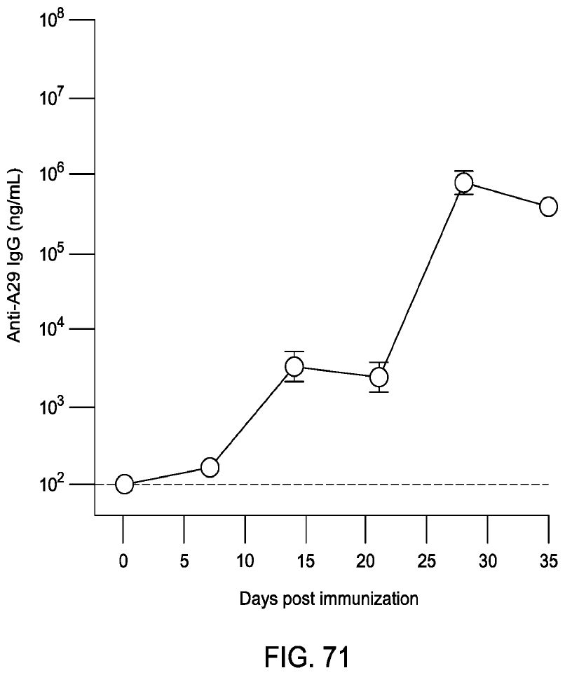

The present Example describes immunogenicity studies of various exemplary polyribonucleotide constructs encoding exemplary monkeypox CSP antigens, alone or in combination. Specifically, an immunization protocol as depicted in the schematic of FIG. 60 was used. In brief, 6 to 8-week-old BALB/c mice were immunized intramuscularly with individual or combination mRNA vaccines on days 0 and 21. For single antigen immunizations, 1 μg of mRNA was administered. Combination vaccines comprised 1 μg of mRNA of each antigen for a total of 2 to 4 μg mRNA. Serum samples were collected weekly until the animals were sacrificed on day 35. Antibody levels were determined by ELISA and germinal center induction in the draining lymph nodes analyzed by flow cytometry. Antibody responses are depicted in FIGS. 61A-61B, FIGS. 62A-62B, FIGS. 63A-63B, FIGS. 64A-64B, FIG. 65, FIGS. 66A-66B, FIG. 67, FIGS. 68A-68B, FIGS. 68A-69B, FIGS. 70A-70B, and FIG. 71.

These results demonstrate that various exemplary polyribonucleotide constructs encoding monkeypox CSP antigens described herein induce an antibody response.

### Example 22: Exemplary Monkeypox Neutralization Assays

The present Example describes neutralization studies of various exemplary polyribonucleotide constructs encoding exemplary monkeypox CSP antigens, alone or in combination.

Specifically, an assay for neutralizing antibodies against MPXV was conducted on serum samples. Vero E6 cells (ATCC, Cat. #CRL-1586) were prepared in plate-seeding media (DMEM+Glutamax+10% FBS+Penicillin/Streptomycin) concentration of cells in suspension was confirmed (e.g., using the Trypan Blue Dye Exclusion Method and visual cell counting under a microscope). Plates were incubated at 37° C., 5% CO₂ until the cells reached 90-100% confluency the following day.

On the day of the assay, serum test samples and controls were heat-inactivated at 56° C. for 30 minutes and separate 96-well dilution plates for controls and serum samples were prepared as described below.

For the control dilution plate setup, infection media (DMEM+Glutamax+2% FBS) was added to the wells. After all infection media was added to the control dilution plate, negative control serum was added, mixed via pipetting, and then titrated across the plate to yield the following dilutions: 1:10, 1:40, 1:160, 1:640, 1:2560, and 1:10240.

For the serum sample dilution plate setup, infection media was added to the wells and then test serum was added to the wells. The samples were titrated across the plate, yielding the same dilution series for the control plate.

The MPXV virus stock dilution was prepared and appropriately diluted in infection media. MPXV virus stock dilution was added to each well of the prepared dilution plates, except for the media/cell control wells. Each well was mixed via pipetting and the plates were incubated for 15-18 hours at 2-8° C.

The following day, the plate-seeding media was removed from 90-100% confluent 24-well plates by decanting. The serum/virus mixtures incubated in the 96-well dilution plates were added to the 24-well seeded plates and then incubated 37° C., 5% CO₂ for 1 hour. During the incubation, the plates were rocked gently every 10-15 minutes to ensure even distribution of the serum/virus mixtures across the cells. A 0.5% methylcellulose overlay medium (0.5% methylcellulose+DMEM+Glutamax+10% FBS+Penicillin/Streptomycin) was prepared and warmed in a 37° C. water bath. Once incubation was complete, pre-warmed overlay medium was added to each well and the plates were incubated at 37° C., 5% CO₂ for 48 hours.

Once the infection step was complete, the overlay medium was removed from the plates by decanting and blotting on absorbent material. A 0.4% Crystal Violet stain solution was added to each well, and the plates were incubated at room temperature for 30-120 minutes. After staining, the Crystal Violet solution removed from the plates by decanting and the plates were inverted set aside to dry.

Dried plates were scanned with a flatbed scanner and desktop scanning software to create digital images for each plate, which were used for manual counting of plaques in each well. The plaque counts were recorded on PRNT Assay Process Sheets (BIOQUAL Lab Form No. 135). PRNT50 titers were calculated based on the average number of plaques detected in the virus control wells.

Complement neutralization assay was performed as described above, but incubation of antibody and virus was conducted with 10% baby rabbit complement (Cedarlane).

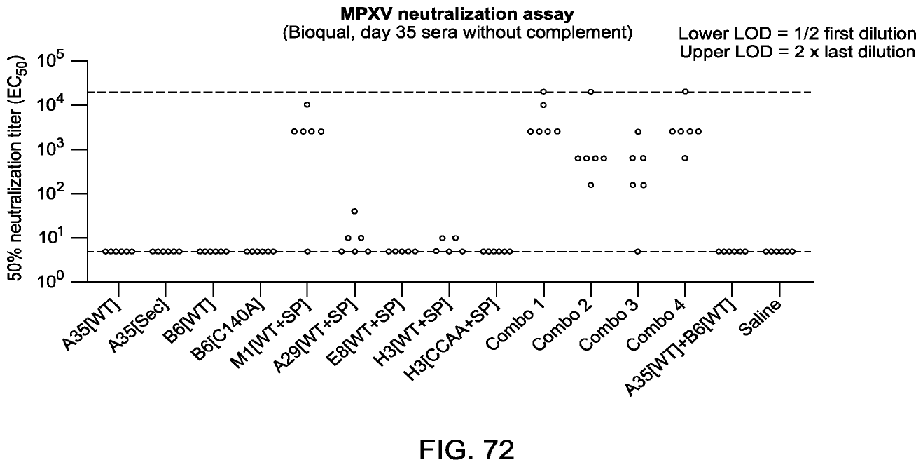

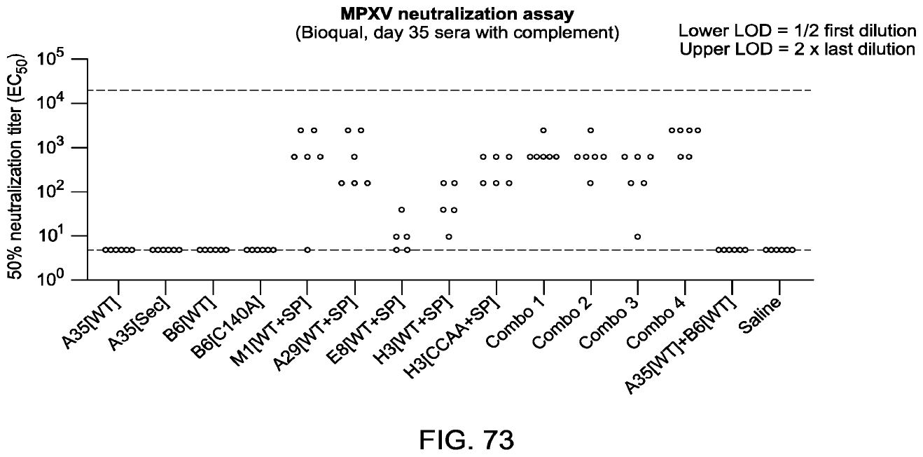

Results of the neutralization assays without and with complement are depicted in FIG. 72 and FIG. 73, respectively. These results support that constructs encoding IMV-specific antigens are able to successfully neutralize MPXV. For example, these results support strong anti-M1 driven neutralization activity. This is consistent with the idea that MPXV viral preparations are dominated by IMV forms of the MPXV virus.

Thus, these results demonstrate that various exemplary polyribonucleotide constructs encoding monkeypox CSP antigens described herein are able to induce a neutralizing antibody response.

### Example 23: Induction of Germinal Centers

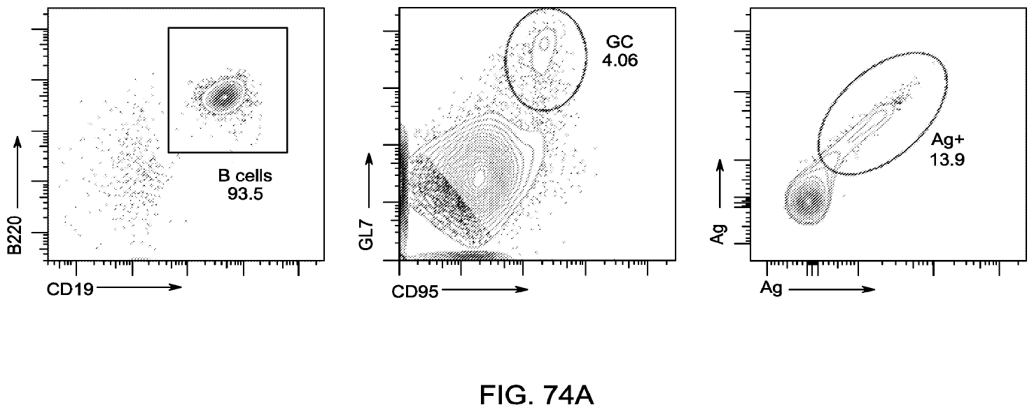

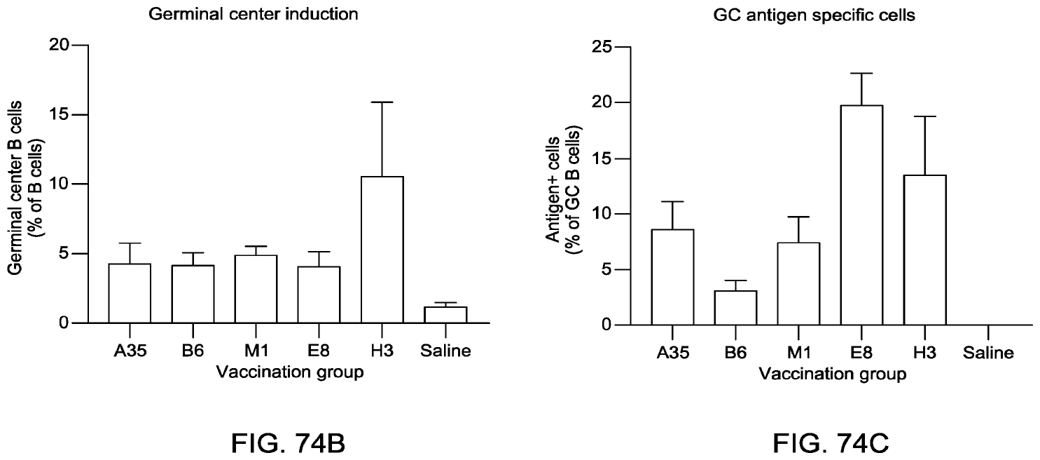

The present Example describes analysis of germinal center induction in lymph nodes from mice immunized with various exemplary polyribonucleotide constructs encoding exemplary monkeypox CSP antigens. Mice were immunized as described in Example 21 and depicted in the schematic of FIG. 60. Inguinal and popliteal lymph nodes (LNs) were harvested from immunized animals 14 days following the second intramuscular vaccine administration. Germinal center induction in these tissues was measured by flow cytometry. Exemplary results are shown in FIG. 74A-74C. FIG. 74A provides exemplary flow cytometry data and these data are summarized in FIG. 74B and FIG. 74C (n=3-6, mean SEM). The total germinal center B cell population as a percent of B cells is shown in FIG. 74B, with the fraction of these B-cells binding antigen shown in FIG. 74C.

These data demonstrate induction of germinal centers and suggests that the exemplary polyribonucleotide constructs encoding exemplary monkeypox CSP antigens described herein generate a durable antibody response.

### Example 24: Exemplary In Vitro Characterization of Vaccine Candidates

The present Example describes non-clinical pharmacological analysis of two exemplary monkeypox polyribonucleotide combination vaccines (Combo 3 and Combo 4) in in vitro studies. As described in the present Example, Combo 3 and Combo 4 are lipid nanoparticle (LNP)-formulated vaccines incorporating modified polyribonucleotides encoding MPXV surface antigens as listed in Table 7.

TABLE 7
Exemplary Monkeypox Combination Vaccine Candidates

| VACCINE | ENCODED EV | ENCODED MV |
|---|---|---|
| CANDIDATE | ANTIGEN | ANTIGEN |
| Combo 1 | B6 | M1 |
| Combo 2 | B6, A35 | M1 |
| Combo 3 | B6, A35 | M1, E8 |
| Combo 4 | B6, A35 | M1, H3 |

Combo 3 or Combo 4, or LNPs containing polyribonucleotides encoding single MPXV antigens (A35, B6, M1, or H3) were transfected into HEK293T cells. The quantity of each polyribonucleotide transfected was equivalent across conditions (100 ng). 18 hours post-transfection, HEK293T cells were harvested, stained with a viability marker and monoclonal antibodies specific for MPXV antigens A35, B6, M1, or H3, and analyzed by flow cytometry.

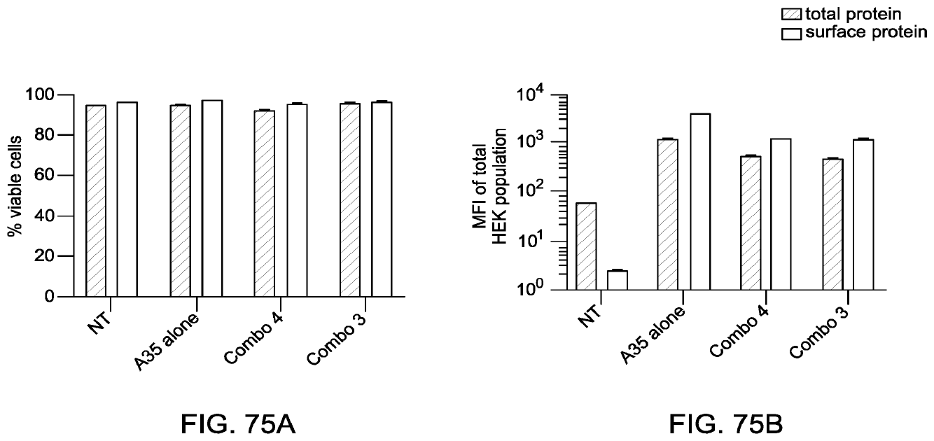

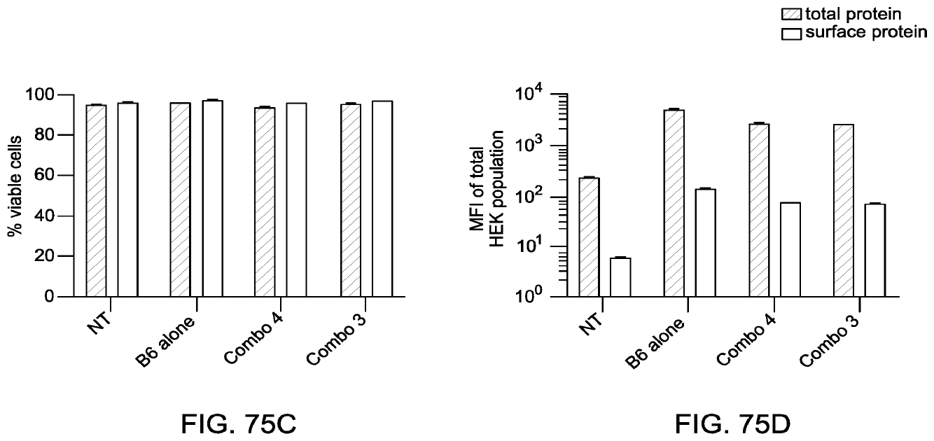

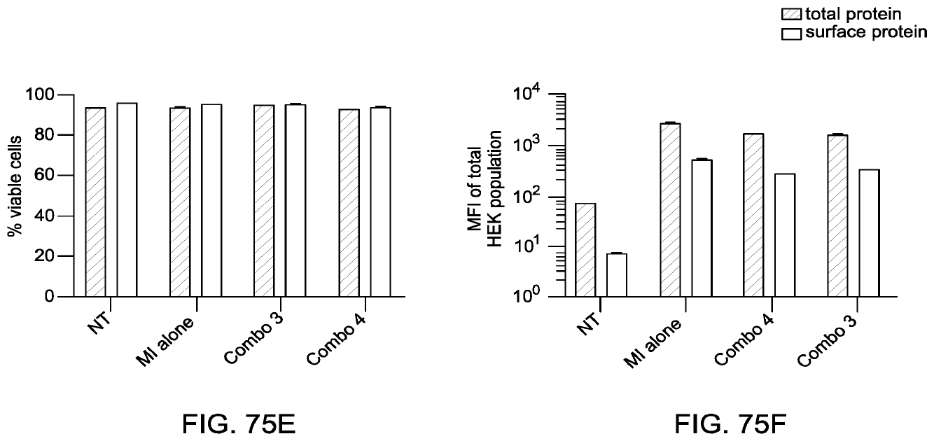

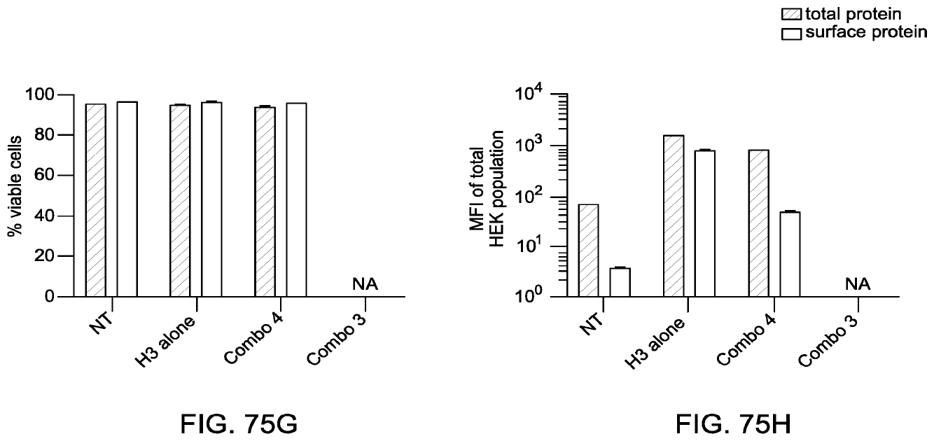

As shown in FIGS. 75A, 75C, 75E, and 75G, high cell viability (>90%) was observed under all transfection conditions. As shown in FIGS. 75B, 75D, 75F, and 75H, polyribonucleotides encoding single MPXV antigens and the polyribonucleotides of Combo 3 and Combo 4 were all successfully translated (total protein) as measured by intracellular staining and flow cytometry analysis. Moreover, in non-permeabilized cells, surface expression analysis confirmed that all translated MPXV antigens correctly localized to the cell membrane (surface protein). Surface expression levels for each MPXV antigen in cells transfected with polyribonucleotides encoding single MPXV antigens and component polyribonucleotides of Combo 3 and Combo 4 were comparable. This data demonstrated that co-administration of multiple polyribonucleotide components did not interefere with the translation of each individual modified polyribonucleotide.

### Example 25: Exemplary In Vivo Characterization of Vaccine Candidates

The present Example describes non-clinical pharmacological analysis of exemplary monkeypox polyribonucleotide combination vaccines (e.g., Combo 1, Combo 2, Combo 3, or Combo 4) in in vivo mouse studies. As described in the present Example, Combo 1, Combo 2, Combo 3, and Combo 4 were lipid nanoparticle (LNP)-formulated vaccines incorporating modified polyribonucleotides encoding MPXV surface antigens as listed in Table 7, above.

### Antibody Induction and ELISA

3 μg of Combo 3, 4 μg of Combo 4, or LNPs incorporating 1 μg of polyribonucleotide encoding a single MPXV antigen (A35, B6, M1, or H3) was intramuscularly administered to the flank region of BalB/C mice on days 0 and 21. Component polyribonuleotides of Combo 3 and Combo 4 were mixed equally by mass such that mice treated with candidate MPXV vaccines each received 1 μg of each polyribonucleotide encoding the relevant MPXV antigen. Blood samples were collected weekly until day 35 and analyzed by antibody immunoassay.

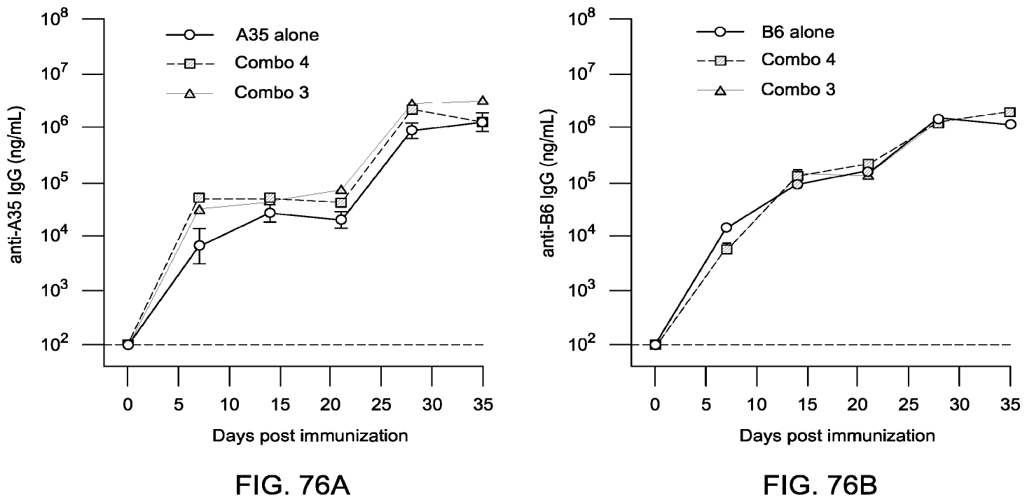

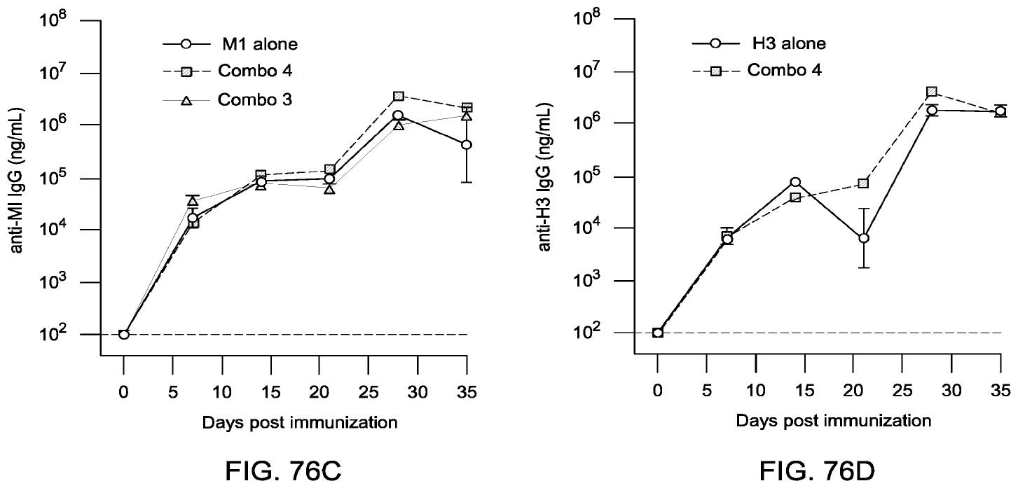

Collected blood samples were analyzed for total immunoglobulin G (IgG) raised to each MPXV antigen by enzyme-linked immunosorbent assay (ELISA). As shown in FIGS. 76A-76D, robust induction of antibody production against MPXV antigens A35 (FIG. 76A), B6 (FIG. 76B), M1 (FIG. 76C), and H3 (FIG. 76D) was observed following the prime dose at day 0 and further increased after the day 21 booster dose in all immunized mice. Comparable serum antibody levels were observed following immunizations with Combo 3, Combo 4, and polyribonucleotides encoding single MPXV antigens. These results suggest that there was no reduction in response to each MPXV antigen target in the context of candidate vaccines delivering multiple antigens.

### Neutralizing Activity and PRNT Assay

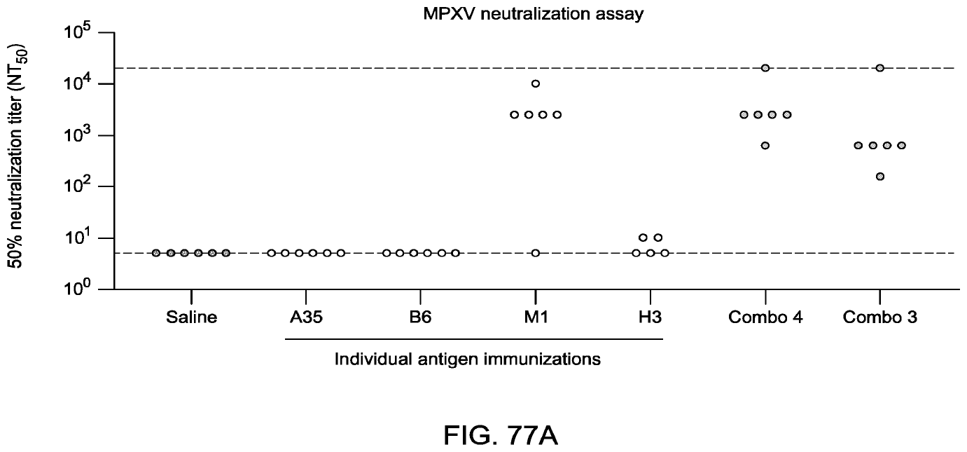

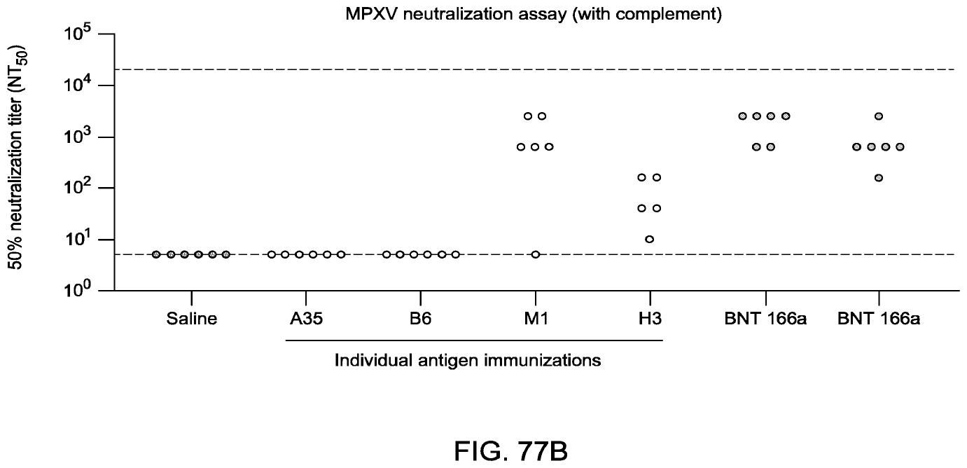

Following confirmation of induction of antibody production for each MPXV antigen, day 35 serum samples were analyzed for MPXV-neutralizing activity by plaque reduction neutralization test (PRNT). As shown in FIGS. 77A and 77B, PRNT assays were performed in the absence of complement (FIG. 77A) and in the presence of 10% baby rabbit complement (FIG. 77B).

Orthopoxvirus neutralization assays present unique complexities arising from the production of two infectious forms of orthopoxvirus, the EEV and IMV. Classic PRNT assays employ virus preparations dominated by IMV virions. Therefore, sera from mice immunized with single EEV antigen targets are not expected to neutralize virus in this context. Consistent with this expectation, in the absence (FIG. 77A) and presence of complement (FIG. 77B), sera from Balb/C mice immunized with polyribonucloeitdes encoding EEV proteins A35 and B6 alone did not show MPXV-neutralizing activity in the PRNT assays. In contrast, sera from mice immunized with polyribonucleotides encoding IMV proteins M1 and H3, efficiently neutralized MPXV both in the absence (FIG. 77A) and presence (FIG. 77B) of complement. Similarly, sera from mice immunized with Combo 3 or Combo 4, efficiently neutralized MPXV both in the absence (FIG. 77A) and presence (FIG. 77B) of complement.

### Antigen-Specific T Cell Induction and ELIspot Assay

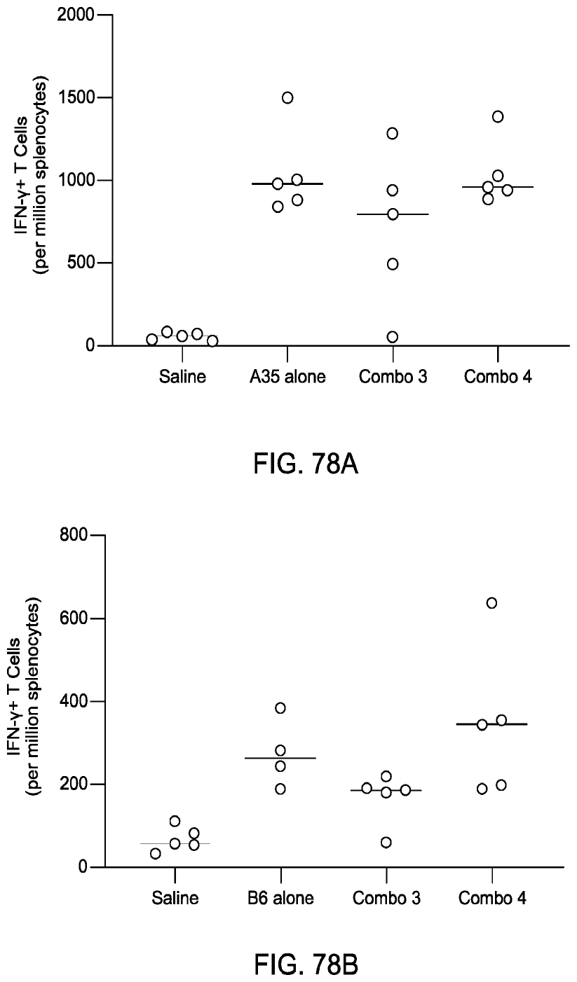

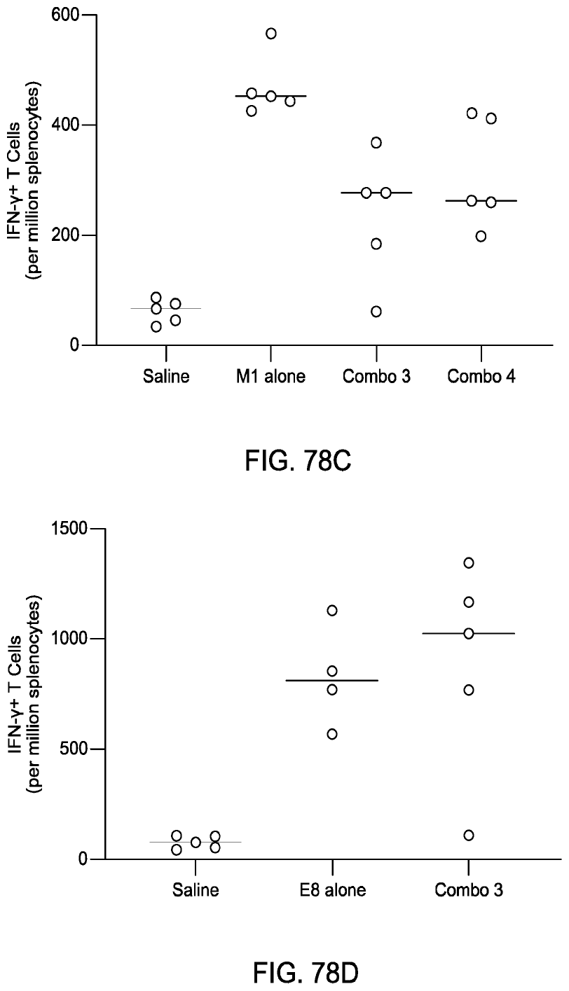

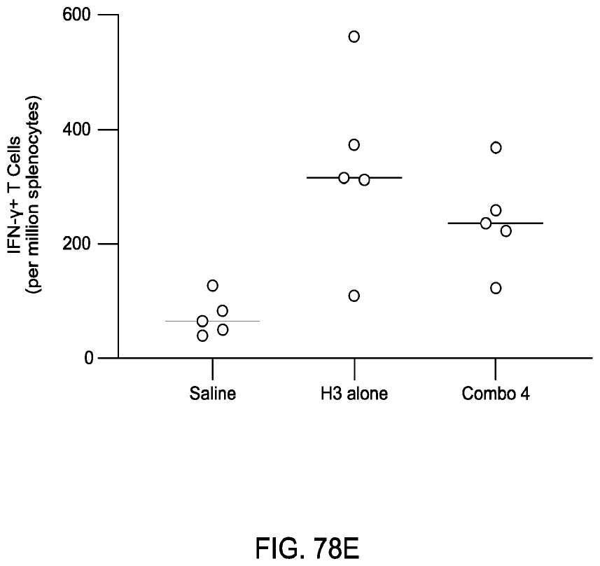

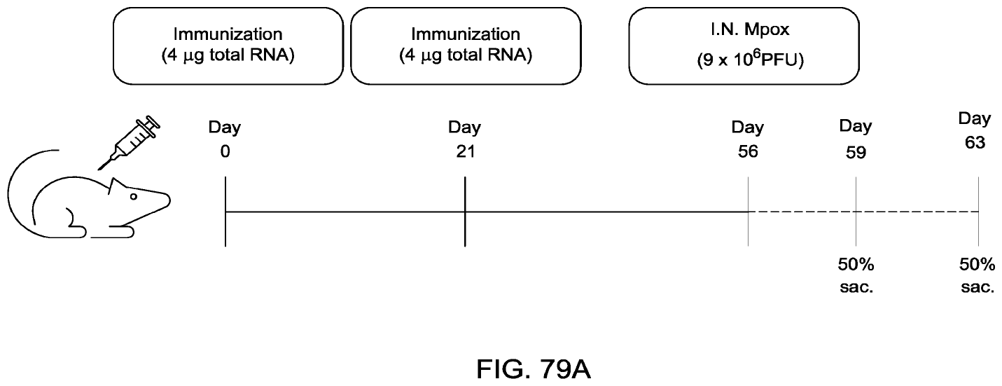

As shown in FIGS. 78A-78E, splenic T cell responses were evaluated following a single intramuscular immunization of 4 μg of Combo 3 or Combo 4, or LNPs incorporating 1 μg of polyribonucleotide encoding a single MPXV antigen. At day 7 post-immunization, mice were sacrificed, spleens collected, and T cell responses measured using IFNγ ELISpot. As shown in FIGS. 78A-78E, T cell responses (i.e. IFNγ expression) specific for MPXV antigens A35 (FIG. 78A), B6 (FIG. 78B), M1 (FIG. 78C), E8 (FIG. 78D), and H3 (FIG. 78E) above levels observed in mock (i.e. saline) immunized animals were detected in splenocytes collected from mice administered with Combo 3 or Combo 4. These results demonstrate that immunization of mice with Combo 3 or Combo 4 led to an increased immune response.

### In Vivo MPXV Challenge in CAST/Ei Mice

Many mouse lines, including the commonly used BALB/c and C57BL/6 lines, are resistant to MPXV infection. However, the immunocompetent inbred mouse line CAST/Ei has been identified as being susceptible to intranasal MPXV challenge. Infection of CAST/Ei mice with a highly pathogenic clade I MPXV isolate (Congo Basin) results in a lethal infection of CAST/Ei (Americo et al., J Virol, 2010), whereas infection with a clade II isolate from the 2022 monkeypox outbreak (SP2833) produces a non-lethal infection with high viral replication rates in the lungs of infected animals (Warner et al. Sci TranslMed, 2022).

In the present Example, CAST/Ei mice were immunized with Combo 1, Combo 2, Combo3, Combo 4, an LNP incorporating a combination of polyribonucleotides encoding A35 and B6, or saline, on days 0 and 21. 5 weeks following the dose at day 21, mice were intranasally challenged with 9×10⁶ plaque forming units (PFUs) of 2022 clade IIb MPXV isolate (hMPXV/USA/MA001/2022). The mice were sacrificed at either day 3 or day 7-post infection, lung samples collected and homogenized, and MPXV titers measured by plaque assay.

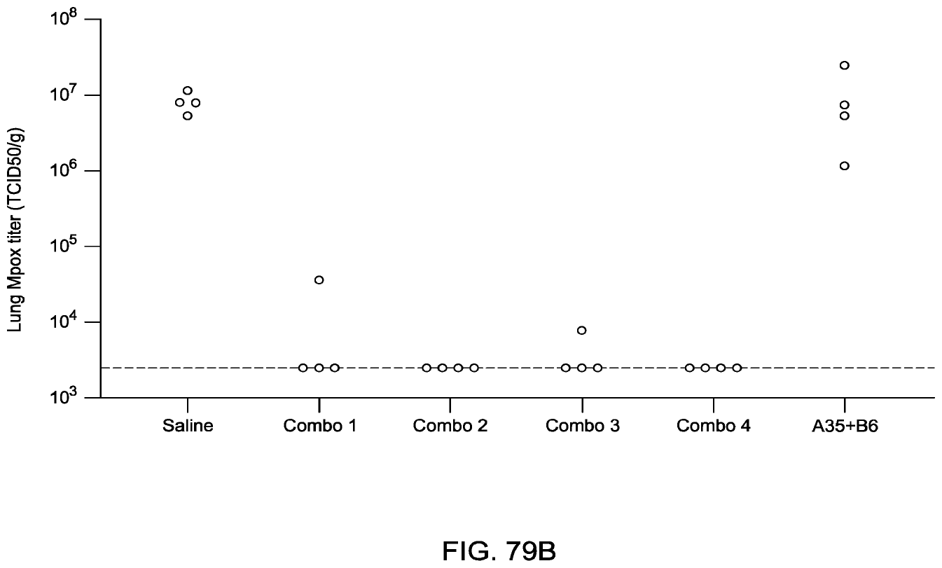

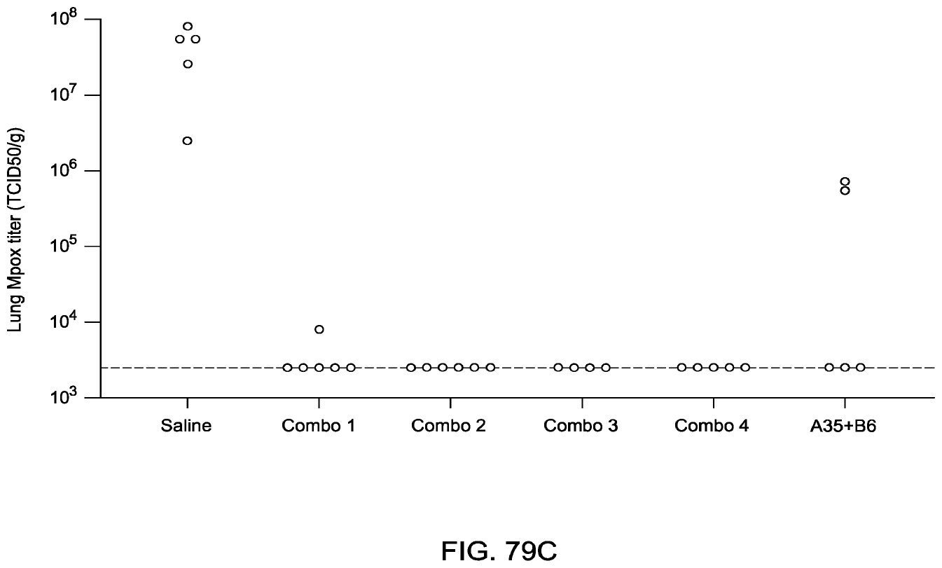

As shown in FIGS. 79A and 79B, saline immunized animals had high viral loads in the lungs at both days 3 and 7, respectively. In contrast, mice immunized Combo 1, Combo 2, Combo 3, or Combo 4 had little to no measurable viral titers at either day 3 (FIG. 79A) or day 7 (FIG. 79B) post-infection. As shown in FIG. 79B, mice immunized with the combination of polyribonucleotides encoding A35 and B6 had a significantly reduced viral load only at day 7-post infection. The reduction of viral loads in the lungs of immunized mice demonstrate that immunization of mice with Combo 1, Combo 2, Combo 3, or Combo 4 provided protection against MPXV.

### Example 26: Exemplary In Vivo Characterization of Broad Orthopoxvirus Protection

The present Example describes non-clinical pharmacological analysis of broad orthopoxvirus protection conferred by exemplary monkeypox polyribonucleotide combination vaccines (e.g., Combo 1, Combo 2, Combo 3, or Combo 4) in in vivo mouse studies. As described in the present Example, Combo 1, Combo 2, Combo 3, and Combo 4 are lipid nanoparticle (LNP)-formulated vaccines incorporating modified polyribonucleotides encoding MPXV surface antigens as listed in Table 7, above.

### Antibody Induction and EELISA

Balb/C mice are immunized with candidate MPX vaccines (e.g., Combo 3 or Combo 4) or LNPs incorporating 1 μg of polyribonucleotide encoding a single MPXV antigen (e.g., A35, B6, M1, E8, or H3) intramuscularly to the flank region of BalB/C mice on days 0 and 21, as previously described in Example 25. Blood samples are collected weekly until day 35.

### Neutralizing Activity and PRNT Assay

Day 35 serum samples are analyzed for orthopoxvirus-neutralizing activity (i.e. ability to neutralize variola virus, vaccinia virus, or cowpox infection) by plaque reduction neutralization test (PRNT) in the absence or presence of complement as previously described in Example 25.

### Antigen-Specific T Cell Induction and ELIspot Assay

Splenic T cell responses are evaluated following a single intramuscular immunization of candidate MPXV vaccine (e.g., Combo 3 or Combo 4) or 1 μg of polyribonucleotide encoding a single MPXV antigen as previously described in Example 25. At day 7 post-immunization, mice are sacrificed, spleens collected, and T cell responses to orthopoxvirus antigens (e.g., variola virus, vaccinia virus, or cowpox antigens) are measured using IFNγ ELISpot.

### In Vivo MPXV Challenge in CAST/Ei Mice

CAST/Ei mice are immunized with candidate MPXV vaccines (e.g., Combo 1, Combo 2, Combo3, Combo 4) or an LNP incorporating a combination of polyribonucleotides encoding A35 and B6, or saline, on days 0 and 21, as previously described in Example 25. 5 weeks following the dose at day 21, mice are intranasally challenged with orthopoxvirus isolates (e.g., variola virus, vaccinia virus, or cowpox isolates). The mice are sacrificed at either day 3 or day 7-post infection, lung samples collected and homogenized, and orthopoxvirus titers measured by plaque assay.

### Example 27: Exemplary Characterization of Immunoglobulin Profiles Elicited by Vaccine Candidates

The present Example describes characterizing the immunoglobulin isotype profiles induced by exemplary monkeypox polyribonucleotide combination vaccines (e.g., Combo 1, Combo 2, Combo 3, or Combo 4) in in vivo mouse studies.

Balb/C mice are immunized with candidate MPX vaccines (e.g., Combo 3 or Combo 4) or LNPs incorporating 1 μg of polyribonucleotide encoding a single MPXV antigen (e.g., A35, B6, M1, E8, or H3) intramuscularly to the flank region of BalB/C mice on days 0 and 21, as previously described in Example 25. Blood samples are collected weekly until day 35.

MPXV antigens are coupled to fluorescently bar-coded beads (e.g., Luminex Corporation) per the manufacturer's instructions. In brief, beads are incubated with diluted, heat-inactivated serum samples prepared from the collected blood samples from immunized mice for 2 hours at 37° C. To detect total IgG, IgG1, IgG2A, IgG2B, IgG3 and FcγR (i.e. FcγR1, FcγR2a, FcγR2b, FcγR3, FcγR4) binding, samples are incubed with beads at a dilution previously determined to be optimal via dilution curve. Beads are washed to remove unbound sample and incubated with fluorescently-labeled detection reagents. Unbound detection reagent is washed away and samples quantified using a Luminex multiplex instrument.

### Example 28: Exemplary Characterization of Macrophage and Neutrophil Phagocytosis Elicited by Vaccine Candidates

The present Example describes characterizing the modulation of macrophage and neutrophil phagocytic activity induced by exemplary monkeypox polyribonucleotide combination vaccines (e.g., Combo 1, Combo 2, Combo 3, or Combo 4) in in vivo mouse studies.

Balb/C mice are immunized with candidate MPXV vaccines (e.g., Combo 3 or Combo 4) or LNPs incorporating 1 μg of polyribonucleotide encoding a single MPXV antigen (e.g., A35, B6, M1, E8, or H3) intramuscularly to the flank region of BalB/C mice on days 0 and 21, as previously described in Example 25. Blood samples are collected weekly until day 35.

MPXV antigens (e.g., A35, B6, M1, E8, or H3) are biotinylated and coupled to fluorescently-labeled streptavidin beads. Antigen-coupled beads are incubated with diluted serum samples prepared from the collected blood samples from immunized mice for 2 hours at 37° C. then washed to remove unbound sample. Macrophage cells (e.g. J774-1 or RAW264.7 cells) are added to immune complexes and incubated for 1 hour at 37° C. Cells are washed to remove unbound immune complexes, fixed, and quantified using a flow cytometer. Phagocytic index is calculated by multiplying the percentage of fluorescently positive cells by the geometric mean fluorescence intensity (MFI) of fluorescently positive cells.

To assess induction of neutrophil phagocytic activity, coated beads are incubated with serum samples for 2 hours at 37° C. then washed to remove unbound antibodies. Bone marrow-derived white blood cells are isolated from C57Bl/6 mice and incubated with immune complexes at 1 hour at 37° C. Cells are washed to remove unbound immune complexes, stained with fluorescently-labeled anti-CD66b antibody, fixed, and quantified using a flow cytometer. Neutrophil phagocytic index is calculated by multiplying the percentage of fluorescently positive CD11b+/Ly6G+ cells by the geometric MFI of fluorescently positive CD11b+/Ly6G+ cells.

### Example 29: Exemplary Characterization of Antibody-Dependent Cell-Mediated Cytotoxicity Elicited by Vaccine Candidates

The present Example describes characterizing the induction of antibody-dependent cell-mediated cytotoxicity (ADCC) induced by exemplary monkeypox polyribonucleotide combination vaccines (e.g., Combo 1, Combo 2, Combo 3, or Combo 4) in in vivo mouse studies.

Balb/C mice are immunized with candidate MPXV vaccines (e.g., Combo 3 or Combo 4) or LNPs incorporating 1 μg of polyribonucleotide encoding a single MPXV antigen (e.g., A35, B6, M1, E8, or H3) intramuscularly to the flank region of BalB/C mice on days 0 and 21, as previously described in Example 25. Blood samples are collected weekly until day 35.

Cells (e.g., Chinese hamster ovary or HEK293T cells) are transfected with DNA expressing membrane-bound MPXV antigens (e.g., A35, B6, M1, E8, or H3). Cells are incubated for 48 hours at 37° C., 5% CO₂. Serum is prepared from the collected blood samples from immunized mice, serially diluted, and added to transfected cells. FcgRIV expressing T cells (e.g., Jurkat cells) are diluted, added to the transfected cells and serum, and incubated for 6 hours at 37° C., 5% CO₂. Luciferase substrate is added to samples and immediately measured using a luminometer.

### Example 30: Exemplary Characterization of Vaccinia Virus Neutralizing Capacity of Sera Derived from Mice Vaccinated with Exemplary Vaccine Candidates

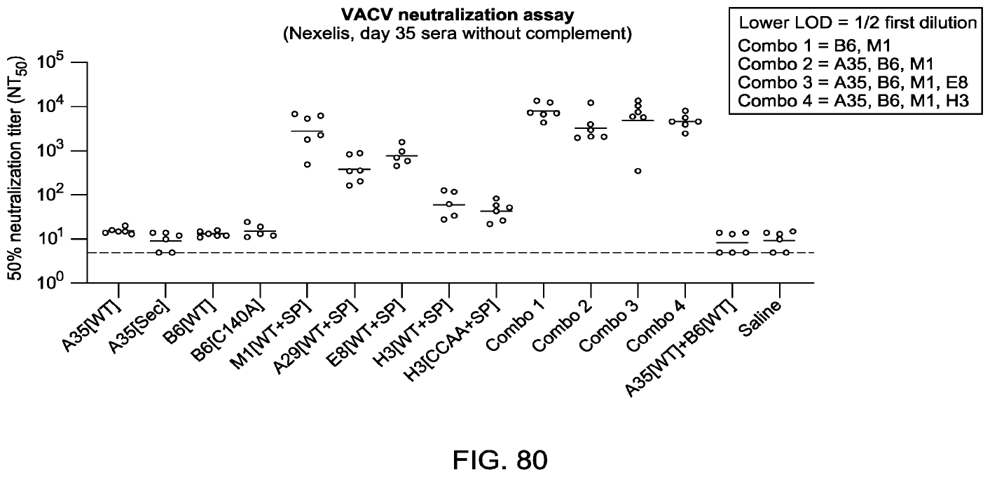

The present example demonstrates vaccinia virus (VACV) neutralizing capacity of sera derived from BALB/c mice immunized with exemplary vaccine candidates, as described herein. The Vaccinia Virus (VACV) Plaque Reduction Neutralization Test (PRNT) is an assay allowing the quantification of neutralizing antibodies against Vaccinia Virus in mouse serum samples. Following confirmation of induction of antibody production for each MPXV antigen, day 35 serum samples were analyzed for VACV-neutralizing (tissue culture-adapted VACV western reserve (ATCC VR-1354)) activity by plaque reduction neutralization test (PRNT). Results of this assay are shown in FIG. 80.

Seven (7) two-fold serial dilutions of heat-inactivated serum samples were performed in a 96-well plate. Once diluted, a standardized amount of VACV was added to the plate and incubated with serum to allow binding of the antibodies to the virus. After a 60 ±5 minutes incubation at 37±2° C. with 5±1% CO₂, the serum-virus complex was transferred to a 96-well plate containing Vero cells, and the plate was further incubated for 60 ±5 minutes at 37±2° C. with 5±1% CO₂. Once the incubation was completed, the serum-virus complex was removed and virus maintenance medium containing 0.5% methylcellulose was added to the plates, which were incubated for 18 hours at 33±2° C. with 5±1% CO₂. Infected Vero cells were then fixed with 4% paraformaldehyde and stained with detection antibodies to read the plate on an automated reader. Images from the automated plate reader allowed the determination of the titer result for each serum sample using the Plaque-Forming Units (PFU) counts. The neutralizing titer of a serum sample was calculated as the reciprocal serum dilution corresponding to the 50% neutralization antibody titer (NT50) for that sample.

Sera collected from Balb/C mice independently immunized with vaccine M1 (WT and SP), A29 (WT and SP), E8 (WT and SP), H3 (WT and SP), H3 (CCAA and SP), Combo 1, Combo 2, Combo 2, and Combo 4 showed VACV-neutralizing activity relative to Saline control. Sera from Balb/C mice immunized with exemplary vaccine candidates Combo 1, Combo 2, Combo 3, and Combo 4 showed the highest VACV-neutralizing activity (FIG. 80). CCAA+SP refers to an H3 antigen having a secretion signal and C86A and C90A substitutions. WT represents the Wild type version of the antigen, and SP represents a version of the antigen with a non-wild type signal peptide or secretion signal. Combo 1 comprises B6 and M1 antigens; Combo 2 comprises A35, B6, and M1 antigens; Combo 3 comprises A35, B6, M1, and E8 antigens; Combo 4 comprises A35, B6, M1, and H3 antigens; and Combo 5 comprises A35 and B6 antigens.

### Example 31: Exemplary Characterization of In-Vivo Efficacy of Exemplary Vaccine Candidates in Mice Infected with VACV

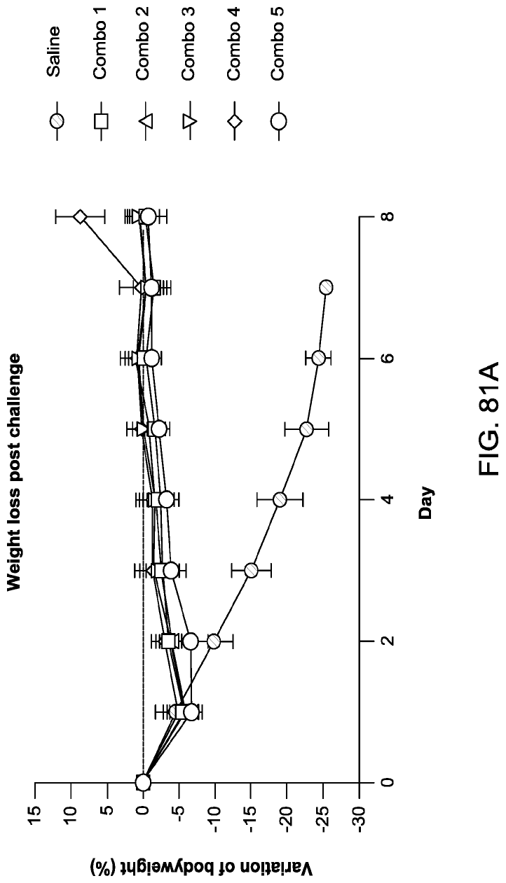

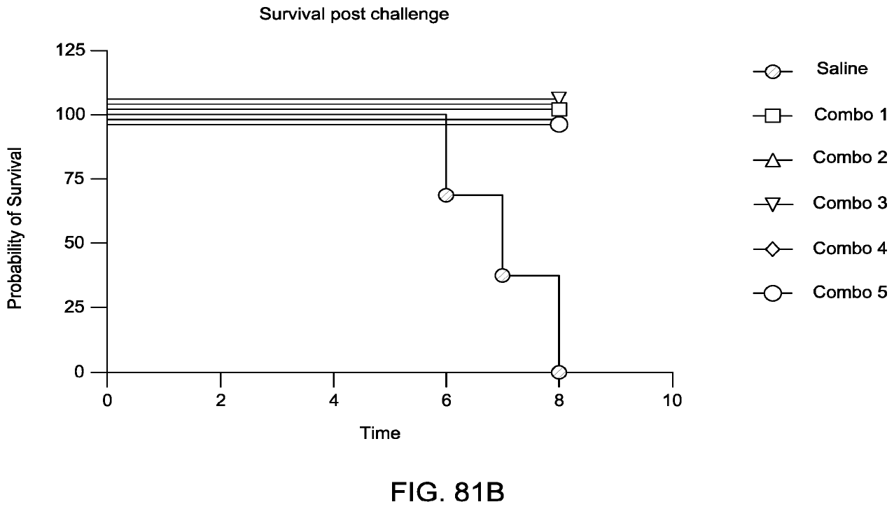

The present example demonstrates the in-vivo efficacy of exemplary vaccine candidates, as described herein. BALB/c mice were immunized intramuscularly (IM) with 4 μg of Combo 1, Combo 2, Combo 3, Combo 4, or Combo 5 mRNA vaccines or saline alone on days 0 and 21. On day 35, mice were challenged intranasally (IN) with a high dose (107 PFU) of tissue culture-adapted VACV western reserve (ATCC VR-1354). Mice were monitored daily for weight loss and survival. BALB/c immunized with exemplary vaccine candidates Combo 1, Combo 2, Combo 3, Combo 4, and Combo 5, survived and increased in weight after VACV challenge (FIGS. 81A and 81B). Mice immunized with saline control showed 100% mortality and lost weight 8 days post VACV challenge. These results demonstrate that exemplary vaccine candidates, as described herein, have the capability of preventing disease and death from VACV infection in a mouse model. Combo 1 comprises B6 and M1 antigens; Combo 2 comprises A35, B6, and M1 antigens; Combo 3 comprises A35, B6, M1, and E8 antigens; Combo 4 comprises A35, B6, M1, and H3 antigens; and Combo 5 comprises A35 and B6 antigens.

TABLE 8
Table of Exemplary Antigen Sequences

| | | Accession, | |
|---|---|---|---|
| SEQ | | Source, or | |
| ID NO | Antigen | Genome | Amino Acid Sequence |
| 1 | A29L | Outbreak | MDGTLFPGDDDLAIPATEFFSTKAAKNPETKREAIVKAYGDDNEETLKQ |
| | | Consensus | RLTNLEKKITNITTKFEQIEKCCKRNDEVLFRLENHAETLRAAMISLAKKI |
| | | | DVQTGRHPYE |
| 2 | A29L | Portuguese | MDGTLFPGDDDLAIPATEFFSTKAAKNPETKREAIVKAYGDDNEETLKQ |
| | | Outbreak | RLTNLEKXITNITTKFEQIEKCCKRNDEVLFRLENHAETLRAAMISLAKKI |
| | | (Portugal20 | DVQTGRHPYE |
| | | 220504) | |
| 3 | A29L | Belgian | MDGTLFPGDDDLAIPATEFFSTKAAKNPETKREAIVKAYGDDNEETLKQ |
| | | Outbreak | RLTNLEKKITNITTKFEQIEKCCKRNDEVLFRLENHAETLRAAMISLAKKI |
| | | (Belgium20 | DVQTGRHPYE |
| | | 220513) | |
| 4 | A29L | NC_003310.1 | MDGTLFPGDDDLAIPATEFFSTKAAKNPETKREAIVKAYGDDNEETLKQ |
| | | | RLTNLEKKITNITTKFEQIEKCCKRNDEVLFRLENHAETLRAAMISLAKKI |
| | | | DVQTGRHPYE |
| 5 | A29L | MPXV | MDGTLFPGDDDLAIPATEFFSTKAAKNPETKREAIVKAYGDDNEETLKQ |
| | | (GenBank: | RLTNLEKKITNITTKFEQIEKCCKRNDEVLFRLENHAETLRAAMISLAKKI |
| | | MN648051.1) | DVQTGRHPYE |
| 6 | A29L | Belgian | MDGTLFPGDDDLAIPATEFFSTKAAKNPETKREAIVKAYGDDNEETLKQ |
| | | Outbreak | RLTNLEKKITNITTKFEQIEKCCKRNDEVLFRLENHAETLRAAMISLAKKI |
| | | (Belgium20 | DVQTGRHPYE |
| | | 220513) | |
| 7 | A29L | MPXV | MDGTLFPGDDDLAIPATEFFSTKAAKNPETKREAIVKAYGDDNEETLKQ |
| | | (KJ642617.1) | RLTNLEKKITNITTKFEQIEKCCKRNDEVLFRLENHAETLRAAMISLAKKI |
| | | | DVQTGRHPYE |
| 8 | A29L | MPXV | MDGTLFPGDDDLAIPATEFFSTKAAKNPETKREAIVKAYGDDNEETLKQ |
| | | (KJ642613.1) | RLTNLEKKITNITTKFEQIEKCCKHNDEVLFRLENHAETLRAAMISLAKKI |
| | | | DVQTGRRPYE |
| 9 | A29L | MPXV | MDGTLFPGDDDLAIPATEFFSTKAAKNPETKREAIVKAYGDDNEETLKQ |
| | | (KJ642612.1) | RLTNLEKKITNITTKFEQIEKCCKHNDEVLFRLENHAETLRAAMISLAKKI |
| | | | DVQTGRRPYE |
| 10 | A29L | MPXV | MDGTLFPGDDDLAIPATEFFSTKAAKNPETKREAIVKAYGDDNEETLKQ |
| | | (HM172544.1) | RLTNLEKKITNITTKFEQIEKCCKHNDEVLFRLENHAETLRAAMISLAKKI |
| | | | DVQTGRRPYE |
| 11 | A35R | Outbreak | MMTPENDEEQTSVFSATVYGDKIQGKNKRKRVIGLCIRISMVISLLSMIT |
| | | Consensus | MSAFLIVRLNQCMSANKAAITDSAVAVAAASSTHRKVVSSTTQYDHKES |
| | | | CNGLYYQGSCYILHSDYKSFEDAKANCAAESSTLPNKSDVLTTWLIDYV |
| | | | EDTWGSDGNPITKTTSDYQDSDVSQEVRKYFCT |
| 12 | A35R | Portuguese | MMTPENDEEQTSVFSATVYGDKIQGKNKRKRVIGLCIRISMVISLLSMIT |
| | | Outbreak | MSAFLIVRLNQCMSANKAAITDSAVAVAAASSTHRKVVSSTTQYDHKES |
| | | (Portugal20 | CNGLYYQGSCYILHSDYKSFEDAKANCAAESSTLPNKSDVLTTWLIDYV |
| | | 220504) | EDTWGSDGNPITKTTSDYQDSDVSQEVRKYFCT |
| 13 | A35R | Belgian | MMTPENDEEQTSVFSATVYGDKIQGKNKRKRVIGLCIRISMVISLLSMIT |
| | | Outbreak | MSAFLIVRLNQCMSANKAAITDSAVAVAAASSTHRKVVSSTTQYDHKES |
| | | (Belgium20 | CNGLYYQGSCYILHSDYKSFEDAKANCAAESSTLPNKSDVLTTWLIDYV |
| | | 220513) | EDTWGSDGNPITKTTSDYQDSDVSQEVRKYFCT |
| 14 | A35R | NC_003310.1 | MTPENDEEQTSVFSATVYGDKIQGKNKRKRVIGLCIRISMVISLLSMITMS |
| | | | AFLIVRLNQCMSANKAAITDSAVAVAAASSTHRKVVSSTTQYDHKESCN |
| | | | GLYYQGSCYILHSDYKSFEDAKANCAAESSTLPNKSDVLTTWLIDYVED |
| | | | TWGSDGNPITKTTSDYQDSDVSQEVRKYFCT |
| 15 | A35R | MPXV | MTPENDEEQTSVFSATVYGDKIQGKNKRKRVIGLCIRISMVISLLSMITMS |
| | | (GenBank: | AFLIVRLNQCMSANKAAITDSAVAVAAASSTHRKVVSSTTQYDHKESCN |
| | | MN648051.1) | GLYYQGSCYILHSDYKSFEDAKANCAAESSTLPNKSDVLTTWLIDYVED |
| | | | TWGSDGNPITKTTSDYQDSDVSQEVRKYFCT |
| 16 | A35R | Belgian | MMTPENDEEQTSVFSATVYGDKIQGKNKRKRVIGLCIRISMVISLLSMIT |
| | | Outbreak | MSAFLIVRLNQCMSANKAAITDSAVAVAAASSTHRKVVSSTTQYDHKES |
| | | (Belgium20 | CNGLYYQGSCYILHSDYKSFEDAKANCAAESSTLPNKSDVLTTWLIDYV |
| | | 220513) | EDTWGSDGNPITKTTSDYQDSDVSQEVRKYFCT |
| 17 | A35R | MPXV | MMTPENDEEQTSVFSATVYGDKIQGKNKRKRVIGLCIRISMVISLLSMIT |
| | | (KJ642617.1) | MSAFLIVRLNQCMSANEAAITDSAVAVAAASSTHRKVASSTTQYDHKES |
| | | | CNGLYYQGSCYILHSDYKSFEDAKANCAAESSTLPNKSDVLTTWLIDYV |
| | | | EDTWGSDGNPITKTTSDYQDSDVSQEVRKYFCT |
| 18 | A35R | MPXV | MMTPENDEEQTSVFSATVYGDKIQGKNKRKRVIGLCIRISMVISLLSMIT |
| | | (KJ642613.1) | MSAFLIVRLNQCMSANEAAITDSAVAVAAASSTHRKVASSTTQYDHKES |
| | | | CNGLYYQGSCYILHSDYKSFEDAKANCAAESSTLPNKSDVLTTWLIDYV |
| | | | EDTWGSDGNPITKTTSDYQDSDVSQEVRKYFCT |
| 19 | A35R | MPXV | MMTPENDEEQTSVFSATVYGDKIQGKNKRKRVIGLCIRISMVISLLSMIT |
| | | (KJ642612.1) | MSAFLIVRLNQCMSANEAAITDSAVAVAAASSTHRKVASSTTQYDHKES |
| | | | CNGLYYQGSCYILHSDYKSFEDAKANCAAESSTLPNKSDVLTTWLIDYV |
| | | | EDTWGSDGNPITKTTSDYQDSDVSQEVRKYFCT |
| 20 | A35R | MPXV | MMTPENDEEQTSVFSATVYGDKIQGKNKRKRVIGLCIRISMVISLLSMIT |
| | | (HM172544.1) | MSAFLIVRLNQCMSANEAAITDSAVAVAAASSTHRKVASSTTQYDHKES |
| | | | CNGLYYQGSCYILHSDYKSFEDAKANCAAESSTLPNKSDVLTTWLIDYV |
| | | | EDTWGSDGNPITKTTSDYQDSDVSQEVRKYFCT |
| 21 | B6R | Outbreak | MKTISVVTLLCVLPAVVYSTCTVPTMNNAKLTSTETSFNDKQKVTFTCD |
| | | Consensus | SGYHSLDPNAVCETDKWKYENPCKKMCTVSDYVSELYDKPLYEVNSTM |
| | | | TLSCNGETKYFRCEEKNGNTSWNDTVTCPNAECQPLQLEHGSCQPVKEK |
| | | | YSFGEYMTINCDVGYEVIGVSYISCTANSWNVIPSCQQKCDIPSLSNGLIS |
| | | | GSTFSIGGVIHLSCKSGFTLTGSPSSTCIDGKWNPILPTCVRSNEEFDPVDD |
| | | | GPDDETDLSKLSKDVVQYEQEIESLEATYHIIIMALTIMGVIFLISIIVLVCS |
| | | | CDKNNDQYKFHKLLP |
| 22 | B6R | Portuguese | XXXXXXXXXXXXXXXXXXXXXXXXXXXXXXXXXXXXXXXXXXXXX |
| | | Outbreak | XXXXXXXXXXXXXXXXXXXXXXXXXXXXXXXXXXXXXXXXXXXXX |
| | | (Portugal20 | XXXXXXXXXXXXXXXXXXXXXXXXXXXXXXXXXXXXXXXXXXXXX |
| | | 220504) | XXXXXXXXXXXXXXXXXXXXXXXXXXXXXXXXXXXXXXXXXXXXX |
| | | | XXXXXXXXXXXXXXXXXXXXXXXXXXXXXXXXXXXXXXXXXXXXX |
| | | | XXXXXXXXXPTXVRSNEEFDPVDDGXDDETDLSKLSKDVVQYEQEIESL |
| | | | EATYHIIIMALTIMGVIFLISIIVLVCSCDKNNDQYKFHKLLP |
| 23 | B6R | Belgian | MKTISVVTLLCVLPAVVYSTCTVPTMNNAKLTSTETSFNDKQKVTFTCD |
| | | Outbreak | SGYHSLDPNAVCETDKWKYENPCKKMCTVSDYVSELYDKPLYEVNSTM |
| | | (Belgium20 | TLSCNGETKYFRCEEKNGNTSWNDTVTCPNAECQPLQLEHGSCQPVKEK |
| | | 220513) | YSFGEYMTINCDVGYEVIGVSYISCTANSWNVIPSCQQKCDIPSLSNGLIS |
| | | | GSTFSIGGVIHLSCKSGFTLTGSPSSTCIDGKWNPILPTCVRSNEEFDPVDD |
| | | | GPDDETDLSKLSKDVVQYEQEIESLEATYHIIIMALTIMGVIFLISIIVLVCS |
| | | | CDKNNDQYKFXXXXP |
| 24 | B6R | NC_003310.1 | MKTISVVTLLCVLPAVVYSTCTVPTMNNAKLTSTETSFNDKQKVTFTCD |
| | | | SGYHSLDPNAVCETDKWKYENPCKKMCTVSDYVSELYDKPLYEVNSTM |
| | | | TLSCNGETKYFRCEEKNGNTSWNDTVTCPNAECQPLQLEHGSCQPVKEK |
| | | | YSFGEYMTINCDVGYEVIGVSYISCTANSWNVIPSCQQKCDIPSLSNGLIS |
| | | | GSTFSIGGVIHLSCKSGFTLTGSPSSTCIDGKWNPILPTCVRSNEEFDPVDD |
| | | | GPDDETDLSKLSKDVVQYEQEIESLEATYHIIIMALTIMGVIFLISIIVLVCS |
| | | | CDKNNDQYKFHKLLP |
| 25 | B6R | MPXV | MKTISVVTLLCVLPAVVYSTCTVPTMNNAKLTSTETSFNDKQKVTFTCD |
| | | (GenBank: | SGYHSLDPNAVCETDKWKYENPCKKMCTVSDYVSELYDKPLYEVNSTM |
| | | MN648051.1) | TLSCNGETKYFRCEEKNGNTSWNDTVTCPNAECQPLQLEHGSCQPVKEK |
| | | | YSFGEYMTINCDVGYEVIGVSYISCTANSWNVIPSCQQKCDIPSLSNGLIS |
| | | | GSTFSIGGVIHLSCKSGFTLTGSPSSTCIDGKWNPILPTCVRSNEEFDPVDD |
| | | | GPDDETDLSKLSKDVVQYEQEIESLEATYHIIIMALTIMGVIFLISIIVLVCS |
| | | | CDKNNDQYKFHKLLP |
| 26 | B6R | Belgian | MKTISVVTLLCVLPAVVYSTCTVPTMNNAKLTSTETSFNDKQKVTFTCD |
| | | Outbreak | SGYHSLDPNAVCETDKWKYENPCKKMCTVSDYVSELYDKPLYEVNSTM |
| | | (Belgium20 | TLSCNGETKYFRCEEKNGNTSWNDTVTCPNAECQPLQLEHGSCQPVKEK |
| | | 220513) | YSFGEYMTINCDVGYEVIGVSYISCTANSWNVIPSCQQKCDIPSLSNGLIS |
| | | | GSTFSIGGVIHLSCKSGFTLTGSPSSTCIDGKWNPILPTCVRSNEEFDPVDD |
| | | | GPDDETDLSKLSKDVVQYEQEIESLEATYHIIIMALTIMGVIFLISIIVLVCS |
| | | | CDKNNDQYKFXXXXP |
| 27 | B6R | MPXV | MKTISVVTLLCVLPAVVYSTCTVPTMNNAKLTSTETSFNDKQKVTFTCD |
| | | (KJ642617.1) | SGYHSLDPNAVCETDKWKYENPCKKMCTVSDYVSELYDKPLYEVNSTM |
| | | | TLSCNGETKYFRCEEKNGNTSWNDTVTCPNAECQPLQLEHGSCQPVKEK |
| | | | YSFGEYMTINCDVGYEVIGVSYISCTANSWNVIPSCQQKCDIPSLSNGLIS |
| | | | GSTFSIGGVIHLSCKSGFTLTGSPSSTCIDGKWNPILPTCVRSNEEFDPVDD |
| | | | GPDDETDLSKLSKDVVQYEQEIESLEATYHIIIMALTIMGVIFLISIIVLVCS |
| | | | CDKNNDQYKFHKLL |
| 28 | B6R | MPXV | MKTISVVTLLCVLPAVVYSTCTVPTMNNAKLTSTETSFNDKQKVTFTCD |
| | | (KJ642613.1) | SGYHSLDPNAVCETDKWKYENPCKKMCTVSDYVSELYDKPLYEVNSTM |
| | | | TLSCNGETKYFRCEEKNGNTSWNDTVTCPNAECQPLQLEHGSCQPVKEK |
| | | | YSFGEYMTINCDVGYEVIGVSYISCTANSWNVIPSCQQKCDIPSLSNGLIS |
| | | | GSTFSIGGVIHLSCKSGFTLTGSPSSTCIDGKWNPILPTCVRSNEEFDPVDD |
| | | | GPDDETDLSKLSKDVVQYEQEIESLEATYHIIIMALTIMGVIFLISIIVLVCS |
| | | | CDKNNDQYKFHKLLP |
| 29 | B6R | MPXV | MKTISVVTLLCVLPAVVYSTCTVPTMNNAKLTSTETSFNDKQKVTFTCD |
| | | (KJ642612.1) | SGYHSLDPNAVCETDKWKYENPCKKMCTVSDYVSELYDKPLYEVNSTM |
| | | | TLSCNGETKYFRCEEKNGNTSWNDTVTCPNAECQPLQLEHGSCQPVKEK |
| | | | YSFGEYMTINCDVGYEVIGVSYISCTANSWNVIPSCQQKCDIPSLSNGLIS |
| | | | GSTFSIGGVIHLSCKSGFTLTGSPSSTCIDGKWNPILPTCVRSNEEFDPVDD |
| | | | GPDDETDLSKLSKDVVQYEQEIESLEATYHIIIMALTIMGVIFLISIIVLVCS |
| | | | CDKNNDQYKFHKLLP |
| 30 | B6R | MPXV | MKTISVVTLLCVLPAVVYSTCTVPTMNNAKLTSTETSFNDKQKVTFTCD |
| | | (HM172544.1) | SGYHSLDPNAVCETDKWKYENPCKKMCTVSDYVSELYDKPLYEVNSTM |
| | | | TLSCNGETKYFRCEEKNGNTSWNDTVTCPNAECQPLQLEHGSCQPVKEK |
| | | | YSFGEYMTINCDVGYEVIGVSYISCTANSWNVIPSCQQKCDIPSLSNGLIS |
| | | | GSTFSIGGVIHLSCKSGFTLTGSPSSTCIDGKWNPILPTCVRSNEEFDPVDD |
| | | | GPDDETDLSKLSKDVVQYEQEIESLEATYHIIIMALTIMGVIFLISIIVLVCS |
| | | | CDKNNDQYKFHKLLP |
| 31 | MIR | Outbreak | MGAAASIQTTVNTLSERISSKLEQEANASAQTKCDIEIGNFYIRQNHGCNI |
| | | Consensus | TVKNMCSADADAQLDAVLSAATETYSGLTPEQKAYVPAMFTAALNIQT |
| | | | SVNTVVRDFENYVKQTCNSSAVVDNKLKIQNVIIDECYGAPGSPTNLEFI |
| | | | NTGSSKGNCAIKALMQLTTKATTQIAPRQVAGTGVQFYMIVIGVIILAAL |
| | | | FMYYAKRMLFTSTNDKIKLILANKENVHWTTYMDTFFRTSPMIIATTDIQ |
| | | | N |
| 32 | MIR | Portuguese | MXAAXSIQTTVNTLSERISSKLEQEANASAQTKCDIEIGNFYIRQNHGCNI |
| | | Outbreak | TVKNMCSADADAQLDAVLSAATETYSGLTPEQKAYVPAMFTAALNIQT |
| | | (Portugal20 | SVNTVVRDFXNYVKQTCNSSAVVDNKLKIQNVIIDECYGAPGSPTNLEFI |
| | | 220504) | NTGSSKGNCAIKALMQLTTKATTQIAPRQVAGTGVQFYMIVIGVIILAAL |
| | | | FMYYAKRMLFTSTNDKIKLILXNKENVHWTTYMDTFFRTSPMIIATTDIQ |
| | | | N |
| 33 | MIR | Belgian | MGAAASIQTTVNTLSERISSKLEQEANASAQTKCDIEIGNFYIRQNHGCNI |
| | | Outbreak | TVKNMCSADADAQLDAVLSAATETYSGLTPEQKAYVPAMFTAALNIQT |
| | | (Belgium20 | SVNTVVRDFENYVKQTCNSSAVVDNKLKIQNVIIDECYGAPGSPTNLEFI |
| | | 220513) | NTGSSKGNCAIKALMQLTTKATTQIAPRQVAGTGVQFYMIVIGVIILAAL |
| | | | FMYYAKRMLFTSTNDKIKLILANKENVHWTTYMDTFFRTSPMIIATTDIQ |
| | | | N |
| 34 | MIR | NC_003310.1 | MGAAASIQTTVNTLSERISSKLEQEANASAQTKCDIEIGNFYIRQNHGCNI |
| | | | TVKNMCSADADAQLDAVLSAATETYSGLTPEQKAYVPAMFTAALNIQT |
| | | | SVNTVVRDFENYVKQTCNSSAVVDNKLKIQNVIIDECYGAPGSPTNLEFI |
| | | | NTGSSKGNCAIKALMQLTTKATTQIAPRQVAGTGVQFYMIVIGVIILAAL |
| | | | FMYYAKRMLFTSTNDKIKLILANKENVHWTTYMDTFFRTSPMIIATTDIQ |
| | | | N |
| 35 | MIR | MPXV | MGAAASIQTTVNTLSERISSKLEQEANASAQTKCDIEIGNFYIRQNHGCNI |
| | | (GenBank: | TVKNMCSADADAQLDAVLSAATETYSGLTPEQKAYVPAMFTAALNIQT |
| | | MN648051.1) | SVNTVVRDFENYVKQTCNSSAVVDNKLKIQNVIIDECYGAPGSPTNLEFI |
| | | | NTGSSKGNCAIKALMQLTTKATTQIAPRQVAGTGVQFYMIVIGVIILAAL |
| | | | FMYYAKRMLFTSTNDKIKLILANKENVHWTTYMDTFFRTSPMIIATTDIQ |
| | | | N |
| 36 | MIR | Belgian | MGAAASIQTTVNTLSERISSKLEQEANASAQTKCDIEIGNFYIRQNHGCNI |
| | | Outbreak | TVKNMCSADADAQLDAVLSAATETYSGLTPEQKAYVPAMFTAALNIQT |
| | | (Belgium20 | SVNTVVRDFENYVKQTCNSSAVVDNKLKIQNVIIDECYGAPGSPTNLEFI |
| | | 220513) | NTGSSKGNCAIKALMQLTTKATTQIAPRQVAGTGVQFYMIVIGVIILAAL |
| | | | FMYYAKRMLFTSTNDKIKLILANKENVHWTTYMDTFFRTSPMIIATTDIQ |
| | | | N |
| 37 | MIR | MPXV | MGAAASIQTTVNTLSERISSKLEQEANASAQTKCDIEIGNFYIRQNHGCNI |
| | | (KJ642617.1) | TVKNMCSADADAQLDAVLSAATETYSGLTPEQKAYVPAMFTAALNIQT |
| | | | SVNTVVRDFENYVKQTCNSSAVVDNKLKIQNVIIDECYGAPGSPTNLEFI |
| | | | NTGSSKGNCAIKALMQLTTKATTQIAPRQVAGTGVQFYMIVIGVIILAAL |
| | | | FMYYAKRMLFTSTNDKIKLILANKENVHWTTYMDTFFRTSPMIIATTDIQ |
| | | | N |
| 38 | MIR | MPXV | MGAAASIQTTVNTLSERISSKLEQEANASAQTKCDIEIGNFYIRQNHGCNI |
| | | (KJ642613.1) | TVKNMCSADADAQLDAVLSAATETYSGLTPEQKAYVPAMFTAALNIQT |
| | | | SVNTVVRDFENYVKQTCNSSAVVDNKLKIQNVIIDECYGAPGSPTNLEFI |
| | | | NTGSSKGNCAIKALMQLTTKATTQIAPRQVAGTGVQFYMIVIGVIILAAL |
| | | | FMYYAKRMLFTSTNDKIKLILANKENVHWTTYMDTFFRTSPMIIATTDIQ |
| | | | N |
| 39 | MIR | MPXV | MGAAASIQTTVNTLSERISSKLEQEANASAQTKCDIEIGNFYIRQNHGCNI |
| | | (KJ642612.1) | TVKNMCSADADAQLDAVLSAATETYSGLTPEQKAYVPAMFTAALNIQT |
| | | | SVNTVVRDFENYVKQTCNSSAVVDNKLKIQNVIIDECYGAPGSPTNLEFI |
| | | | NTGSSKGNCAIKALMQLTTKATTQIAPRQVAGTGVQFYMIVIGVIILAAL |
| | | | FMYYAKRMLFTSTNDKIKLILANKENVHWTTYMDTFFRTSPMIIATTDIQ |
| | | | N |
| 40 | MIR | MPXV | MGAAASIQTTVNTLSERISSKLEQEANASAQTKCDIEIGNFYIRQNHGCNI |
| | | (HM172544.1) | TVKNMCSADADAQLDAVLSAATETYSGLTPEQKAYVPAMFTAALNIQT |
| | | | SVNTVVRDFENYVKQTCNSSAVVDNKLKIQNVIIDECYGAPGSPTNLEFI |
| | | | NTGSSKGNCAIKALMQLTTKATTQIAPRQVAGTGVQFYMIVIGVIILAAL |
| | | | FMYYAKRILFTSTNDKIKLILANKENVHWTTYMDTFFRTSPMIIATTDIQN |
| 41 | E8L | Outbreak | MPQQLSPINIETKKAISDARLKTLDIHYNESKPTTIQNTGKLVRINFKGGYI |
| | | Consensus | SGGFLPNEYVLSTIHIYWGKEDDYGSNHLIDVYKYSGEINLVHWNKKKY |
| | | | SSYEEAKKHDDGIIIIAIFLQVSDHKNVYFQKIVNQLDSIRSANMSAPFDSV |
| | | | FYLDNLLPSTLDYFTYLGTTINHSADAAWIIFPTPINIHSDQLSKFRTLLSSS |
| | | | NHEGKPHYITENYRNPYKLNDDTQVYYSGEIIRAATTSPVRENYFMKWL |
| | | | SDLREACFSYYQKYIEGNKTFAIIAIVFVFILTAILFLMSQRYSREKQN |
| 42 | E8L | Portuguese | XPQQLSPINIETKKAISDARLKTLDIHYNESKPTTIXNTGKLVRINXKGGYI |
| | | Outbreak | SGGFLPNEYVLSTIHIYWGKEDDYGSNHLIDVYKYSGEINLVHWNKKKY |
| | | (Portugal20 | SSYEEAKXHDDGIIIIAIFLQVSDHKXVYFQKIVNQLDSIRSANMSAPFDSV |
| | | 220504) | FYLDNLLPSTLDYFTYLGTTINHSADAAWIXXXTPINIHSDQLSKFXTLLS |
| | | | SSNHEGKPHYITXNYRNPYKLNDDTQVYYSGXIXXAATTSPVRENYFMK |
| | | | WLSDLRXACFXYYQKYIEGNKTFAIIAIVFVFILTAILFLXSQRYSRXKON |
| 43 | E8L | Belgian | MPQQLSPINIETKKAISDARLKTLDIHYNESKPTTIQNTGKLVRINFKGGYI |
| | | Outbreak | SGGFLPNEYVLSTIHIYWGKEDDYGSNHLIDVYKYSGEINLVHWNKKKY |
| | | (Belgium20 | SSYEEAKKHDDGIIIIAIFLQVSDHKNVYFQKIVNQLDSIRSANMSAPFDSV |
| | | 220513) | FYLDNLLPSTLDYFTYLGTTINHSADAAWIIFPTPINIHSDQLSKFRTLLSSS |
| | | | NHEGKPHYITENYRNPYKLNDDTQVYYSGEIIRAATTSPVRENYFMKWL |
| | | | SDLREACFSYYQKYIEGNKTFAIIAIVFVFILTAILFLMSQRYSREKQN |
| 44 | E8L | NC_003310.1 | MPQQLSPINIETKKAISDARLKTLDIHYNESKPTTIQNTGKLVRINFKGGYI |
| | | | SGGFLPNEYVLSTIHIYWGKEDDYGSNHLIDVYKYSGEINLVHWNKKKY |
| | | | SSYEEAKKHDDGIIIIAIFLQVSDHKNVYFQKIVNQLDSIRSANMSAPFDSV |
| | | | FYLDNLLPSTLDYFTYLGTTINHSADAAWIIFPTPINIHSDQLSKFRTLLSSS |
| | | | NHEGKPHYITENYRNPYKLNDDTQVYYSGEIIRAATTSPVRENYFMKWL |
| | | | SDLREACFSYYQKYIEGNKTFAIIAIVFVFILTAILFLMSQRYSREKON |
| 45 | E8L | MPXV | MPQQLSPINIETKKAISDARLKTLDIHYNESKPTTIQNTGKLVRINFKGGYI |
| | | (GenBank: | SGGFLPNEYVLSTIHIYWGKEDDYGSNHLIDVYKYSGEINLVHWNKKKY |
| | | MN648051.1) | SSYEEAKKHDDGIIIIAIFLQVSDHKNVYFQKIVNQLDSIRSANMSAPFDSV |
| | | | FYLDNLLPSTLDYFTYLGTTINHSADAAWIIFPTPINIHSDQLSKFRTLLSSS |
| | | | NHEGKPHYITENYRNPYKLNDDTQVYYSGEIIRAATTSPVRENYFMKWL |
| | | | SDLREACFSYYQKYIEGNKTFAIIAIVFVFILTAILFLMSQRYSREKQN |
| 46 | E8L | Belgian | MPQQLSPINIETKKAISDARLKTLDIHYNESKPTTIQNTGKLVRINFKGGYI |
| | | Outbreak | SGGFLPNEYVLSTIHIYWGKEDDYGSNHLIDVYKYSGEINLVHWNKKKY |
| | | (Belgium20 | SSYEEAKKHDDGIIIIAIFLQVSDHKNVYFQKIVNQLDSIRSANMSAPFDSV |
| | | 220513) | FYLDNLLPSTLDYFTYLGTTINHSADAAWIIFPTPINIHSDQLSKFRTLLSSS |
| | | | NHEGKPHYITENYRNPYKLNDDTQVYYSGEIIRAATTSPVRENYFMKWL |
| | | | SDLREACFSYYQKYIEGNKTFAIIAIVFVFILTAILFLMSQRYSREKQN |
| 47 | E8L | MPXV | MPQQLSPINIETKKAISDARLKTLDIHYNESKPTTIQNTGKLVRINFKGGYI |
| | | (KJ642617.1) | SGGFLPNEYVLSTIHIYWGKEDDYGSNHLIDVYKYSGEINLVHWNKKKY |
| | | | SSYEEAKKHDDGIIIIAIFLQVSDHKNVYFQKIVNQLDSIRSANMSAPFDSV |
| | | | FYLDNLLPSTLDYFTYLGTTINHSADAAWIIFPTPINIHSDQLSKFRTLLSSS |
| | | | NHEGKPHYITENYRNPYKLNDDTQVYYSGEIIRAATTSPVRENYFMKWL |
| | | | SDLREACFSYYQKYIEGNKTFAIIAIVFVFILTAILFLMSQRYSREKQN |
| 48 | E8L | MPXV | MPQQLSPINIETKKAISDTRLKTLDIHYNESKPTTIQNTGKLVRINFKGGYI |
| | | (KJ642613.1) | SGGFLPNEYVLSTIHIYWGKEDDYGSNHLIDVYKYSGEINLVHWNKKKY |
| | | | SSYEEAKKHDDGIIIIAIFLQVSDHKNVYFQKIVNQLDSIRSANMSAPFDSV |
| | | | FYLDNLLPSTLDYFTYLGTTINHSADAAWIIFPTPINIHSDQLSKFRTLLSSS |
| | | | NHEGKPHYITENYRNPYKLNDDTQVYYSGEIIRAATTSPVRENYFMKWL |
| | | | SDLREACFSYYQKYIEGNKTFAIIAIVFVFILTAILFLMSQRYSREKQN |
| 49 | E8L | MPXV | MPQQLSPINIETKKAISDTRLKTLDIHYNESKPTTIQNTGKLVRINFKGGYI |
| | | (KJ642612.1) | SGGFLPNEYVLSTIHIYWGKEDDYGSNHLIDVYKYSGEINLVHWNKKKY |
| | | | SSYEEAKKHDDGIIIIAIFLQVSDHKNVYFQKIVNQLDSIRSANMSAPFDSV |
| | | | FYLDNLLPSTLDYFTYLGTTINHSADAAWIIFPTPINIHSDQLSKFRTLLSSS |
| | | | NHEGKPHYITENYRNPYKLNDDTQVYYSGEIIRAATTSPVRENYFMKWL |
| | | | SDLREACFSYYQKYIEGNKTFAIIAIVFVFILTAILFLMSQRYSREKQN |
| 50 | E8L | MPXV | MPQQLSPINIETKKAISDTRLKTLDIHYNESKPTTIQNTGKLVRINFKGGYI |
| | | (HM172544.1) | SGGFLPNEYVLSTIHIYWGKEDDYGSNHLIDVYKYSGEINLVHWNKKKY |
| | | | SSYEEAKKHDDGIIIIAIFLQVSDHKNVYFQKIVNQLDSIRSANMSAPFDSV |
| | | | FYLDNLLPSTLDYFTYLGTTINHSADAAWIIFPTPINIHSDQLSKFRTLLSSS |
| | | | NHEGKPHYITENYRNPYKLNDDTQVYYSGEIIRAATTSPVRENYFMKWL |
| | | | SDLREACFSYYQKYIEGNKTFAIIAIVFVFILTTILFLMSQRYSREKQN |
| 51 | H3L | Outbreak | MAAVKTPVIVVPVIDRPPSETFPNVHEHINDQKFDDVKDNEVMQEKRDV |
| | | Consensus | VIVNDDPDHYKDYVFIQWTGGNIRDDDKYTHFFSGFCNTMCTEETKRNI |
| | | | ARHLALWDSKFFTELENKNVEYVVIIENDNVIEDITFLRPVLKAIHDKKID |
| | | | ILQMREIITGNKVKTELVIDKDHAIFTYTGGYDVSLSAYIIRVTTALNIVDE |
| | | | IIKSGGLSSGFYFEIARIENEMKINRQIMDNSAKYVEHDPRLVAEHRFETM |
| | | | KPNFWSRIGTVAAKRYPGVMYTFTTPLISFFGLFDINVIGLIVILFIMFMLIF |
| | | | NVKSKLLWFLTGTFVTAFI |
| 52 | H3L | Portuguese | MAAVKTPVIVVPVIDRPPSETFPNVHEHINDQKFDDVKDXEVMQEKRDV |
| | | Outbreak | VIVNDDPDHYKDYVFIQWTGGNIRDDDKYTHFFSGFCNTMCTEETKRNI |
| | | (Portugal20 | ARHLALWDSKFXTELENKNVEYVVIIENDNVIEDITFLRPVLKAIHDKKID |
| | | 220504) | ILQMREIITGNKVKTELVXDKDHAIFTYTGGYDVSLSAYIIXVTTALNIVD |
| | | | EIIKSGGLSSGFYFEIARIENEMKINRQIMDNSAKYVEHDPRLVAEHRFET |
| | | | MKPNFWSRIGTVAAKRYPGVMYTFTTPLISFFGLFDINVIGLIVILFIMFML |
| | | | IFNVKSKLLWFLTGTFVTAFI |
| 53 | H3L | Belgian | MAAVKTPVIVVPVIDRPPSETFPNVHEHINDQKFDDVKDNEVMQEKRDV |
| | | Outbreak | VIVNDDPDHYKDYVFIQWTGGNIRDDDKYTHFFSGFCNTMCTEETKRNI |
| | | (Belgium20 | ARHLALWDSKFFTELENKNVEYVVIIENDNVIEDITFLRPVLKAIHDKKID |
| | | 220513) | ILQMREIITGNKVKTELVIDKDHAIFTYTGGYDVSLSAYIIRVTTALNIVDE |
| | | | IIKSGGLSSGFYFEIARIENEMKINRQIMDNSAKYVEHDPRLVAEHRFETM |
| | | | KPNFWSRIGTVAAKRYPGVMYTFTTPLISFFGLFDINVIGLIVILFIMFMLIF |
| | | | NVKSKLLWFLTGTFVTAFI |
| 54 | H3L | NC_003310.1 | MAAVKTPVIVVPVIDRPPSETFPNVHEHINDQKFDDVKDNEVMQEKRDV |
| | | | VIVNDDPDHYKDYVFIQWTGGNIRDDDKYTHFFSGFCNTMCTEETKRNI |
| | | | ARHLALWDSKFFTELENKNVEYVVIIENDNVIEDITFLRPVLKAIHDKKID |
| | | | ILQMREIITGNKVKTELVIDKDHAIFTYTGGYDVSLSAYIIRVTTALNIVDE |
| | | | IIKSGGLSSGFYFEIARIENEMKINRQIMDNSAKYVEHDPRLVAEHRFETM |
| | | | KPNFWSRIGTVAAKRYPGVMYTFTTPLISFFGLFDINVIGLIVILFIMFMLIF |
| | | | NVKSKLLWFLTGTFVTAFI |
| 55 | H3L | MPXV | MAAVKTPVIVVPVIDRPPSETFPNVHEHINDQKFDDVKDNEVMQEKRDV |
| | | (GenBank: | VIVNDDPDHYKDYVFIQWTGGNIRDDDKYTHFFSGFCNTMCTEETKRNI |
| | | MN648051.1) | ARHLALWDSKFFTELENKNVEYVVIIENDNVIEDITFLRPVLKAIHDKKID |
| | | | ILQMREIITGNKVKTELVIDKDHAIFTYTGGYDVSLSAYIIRVTTALNIVDE |
| | | | IIKSGGLSSGFYFEIARIENEMKINRQIMDNSAKYVEHDPRLVAEHRFETM |
| | | | KPNFWSRIGTVAAKRYPGVMYTFTTPLISFFGLFDINVIGLIVILFIMFMLIF |
| | | | NVKSKLLWFLTGTFVTAFI |
| 56 | H3L | Belgian | MAAVKTPVIVVPVIDRPPSETFPNVHEHINDQKFDDVKDNEVMQEKRDV |
| | | Outbreak | VIVNDDPDHYKDYVFIQWTGGNIRDDDKYTHFFSGFCNTMCTEETKRNI |
| | | (Belgium20 | ARHLALWDSKFFTELENKNVEYVVIIENDNVIEDITFLRPVLKAIHDKKID |
| | | 220513) | ILQMREIITGNKVKTELVIDKDHAIFTYTGGYDVSLSAYIIRVTTALNIVDE |
| | | | IIKSGGLSSGFYFEIARIENEMKINRQIMDNSAKYVEHDPRLVAEHRFETM |
| | | | KPNFWSRIGTVAAKRYPGVMYTFTTPLISFFGLFDINVIGLIVILFIMFMLIF |
| | | | NVKSKLLWFLTGTFVTAFI |
| 57 | H3L | MPXV | MAAVKTPVIVVPVIDRPPSETFPNVHEHINDQKFDDVKDNEVMQEKRDV |
| | | (KJ642617.1) | VIVNDDPDHYKDYVFIQWTGGNIRDDDKYTHFFSGFCNTMCTEETKRNI |
| | | | ARHLALWDSKFFTELENKNVEYVVIIENDNVIEDITFLRPVLKAIHDKKID |
| | | | ILQMREIITGNKVKTELVIDKDHAIFTYTGGYDVSLSAYIIRVTTALNIVDE |
| | | | IIKSGGLSSGFYFEIARIENEMKINRQIMDNSAKYVEHDPRLVAEHRFETM |
| | | | KPNFWSRIGTVAAKRYPGVMYTFTTPLISFFGLFDINVIGLIVILFIMFMLIF |
| | | | NVKSKLLWFLTGTFVTAFI |
| 58 | H3L | MPXV | MAAAKTPVIVVPVIDRPPSETFPNVHEHINDQKFDDVKDNEVMQEKRDV |
| | | (KJ642613.1) | VIVNDDPDHYKDYVFIQWTGGNIRDDDKYTHFFSGFCNTMCTEETKRNI |
| | | | ARHLALWDSKFFIELENKNVEYVVIIENDNVIEDITFLRPVLKAIHDKKIDI |
| | | | LQMREIITGNKVKTELVIDKDHAIFTYTGGYDVSLSAYIIRVTTALNIVDEI |
| | | | IKSGGLSSGFYFEIARIENEMKINRQIMDNSAKYVEHDPRLVAEHRFETM |
| | | | KPNFWSRIGTVAAKRYPGVMYTFTTPLISFFGLFDINVIGLIVILFIMFMLIF |
| | | | NVKSKLLWFLTGTFVTAFI |
| 59 | H3L | MPXV | MAAAKTPVIVVPVIDRPPSETFPNVHEHINDQKFDDVKDNEVMQEKRDV |
| | | (KJ642612.1) | VIVNDDPDHYKDYVFIQWTGGNIRDDDKYTHFFSGFCNTMCTEETKRNI |
| | | | ARHLALWDSKFFIELENKNVEYVVIIENDNVIEDITFLRPVLKAIHDKKIDI |
| | | | LQMREIITGNKVKTELVIDKDHAIFTYTGGYDVSLSAYIIRVTTALNIVDEI |
| | | | IKSGGLSSGFYFEIARIENEMKINRQIMDNSAKYVEHDPRLVAEHRFETM |
| | | | KPNFWSRIGTVAAKRYPGVMYTFTTPLISFFGLFDINVIGLIVILFIMFMLIF |
| | | | NVKSKLLWFLTGTFVTAFI |
| 60 | H3L | MPXV | MAAAKTPVIVVPVIDRPPSETFPNVHEHINDQKFDDVKDNEVMQEKRDV |
| | | (HM172544.1) | VIVNDDPDHYKDYVFIQWTGGNIRDDDKYTHFFSGFCNTMCTEETKRNI |
| | | | ARHLALWDSKFFIELENKNVEYVVIIENDNVIEDITFLRPVLKAIHDKKIDI |
| | | | LQMREIITGNKVKTELVIDKDHAIFTYTGGYDVSLSAYIIRVTTALNIVDEI |
| | | | IKSGGLSSGFYFEIARIENEMKINRQIMDNSAKYVEHDPRLVAEHRFETM |
| | | | KPNFWSRIGTVAAKRYPGVMYTFTTPLISFFGLFDINVIGLIVILFIMFMLIF |
| | | | NVKSKLLWFLTGTFVTAFI |
| 61 | A45L | Outbreak | MAVYAVTGGAGFLGRYIVKLLISADDVQEIRVIDIVEDPQPITSKVKVINY |
| | | Consensus | IQCDINDFDKVREALDGVNLIIHTAALVDVFGKYTDNEIMKVNYYGTQTI |
| | | | LAACVDLGIKYLIYTSSMEAIGPNKHGNPFIGHEHTLYDISPGHVYAKSK |
| | | | RMAEQLVMKANNSVIMNGAKLYTCCLRPTGIYGEGDKLTKVFYEQCKQ |
| | | | HGNIMYRTVDDDAVHSRVYVGNVAWMHVLAAKYIQYPGSAIKGNAYF |
| | | | CYDYSPSCSYDMFNLLLMKPLGIEQGSRIPRWMLKMYACKNDMKRILFR |
| | | | KPSILNNYTLKISNTTFEVRTNNAELDFNYSPIFDVDVAFERTRKWLEESE |
| 62 | A45L | Portuguese | MAVYAVTGGAGFLGRYIVKLLISADDVQEIRVIDIVEDPQPITSKVKVINY |
| | | Outbreak | IQCDINDFDKVREALDGVNLIIHTAALVDVFGKYTDNEIMKVNYYGTQTI |
| | | (Portugal20 | LAACVDLGIKYLIYTSSMEAIGPNKHGNPFIGHEHTLYDISPGHVYAKSK |
| | | 220504) | RMAEQLVMKANNSVIMNGAKLYTCCLRPTGIYGEGDKLTKVFYEQCKQ |
| | | | HGNIMYRTVDDDAVHSRVYVGNVAWMHVLAAKYIQYPGSAIKGNAYF |
| | | | CYDYSPSCSYDMFNLLLMKPLGIEQGSRIPRWMLKMYACKNDMKRILFR |
| | | | KPSILNNYTLKISNTTFEVRTNNAELDFNYSPIFDVDVAFERTRKWLEESE |
| 63 | A45L | Belgian | MAVYAVTGGAGFLGRYIVKLLISADDVQEIRVIDIVEDPQPITSKVKVINY |
| | | Outbreak | IQCDINDFDKVREALDGVNLIIHTAALVDVFGKYTDNEIMKVNYYGTQTI |
| | | (Belgium20 | LAACVDLGIKYLIYTSSMEAIGPNKHGNPFIGHEHTLYDISPGHVYAKSK |
| | | 220513) | RMAEQLVMKANNSVIMNGAKLYTCCLRPTGIYGEGDKLTKVFYEQCKQ |
| | | | HGNIMYRTVDDDAVHSRVYVGNVAWMHVLAAKYIQYPGSAIKGNAYF |
| | | | CYDYSPSCSYDMFNLLLMKPLGIEQGSRIPRWMLKMYACKNDMKRILFR |
| | | | KPSILNNYTLKISNTTFEVRTNNAELDFNYSPIFDVDVAFERTRKWLEESE |
| 64 | A45L | NC_003310.1 | MAVYAVTGGAGFLGRYIVKLLISADDVQEIRVIDIVEDPQPITSKVKVINY |
| | | | IQCDINDFDKVREALDGVNLIIHTAALVDVFGKYTDNEIMKVNYYGTQTI |
| | | | LAACVDLGIKYLIYTSSMEAIGPNKHGNPFIGHEHTLYDISPGHVYAKSK |
| | | | RMAEQLVMKANNSVIMNGAKLYTCCLRPTGIYGEGDKLTKVFYEQCKQ |
| | | | HGNIMYRTVDDDAVHSRVYVGNVAWMHVLAAKYIQYPGSAIKGNAYF |
| | | | CYDYSPSCSYDMFNLLLMKPLGIEQGSRIPRWMLKMYACKNDMKRILFR |
| | | | KPSILNNYTLKISNTTFEVRTNNAELDFNYSPIFDVDVAFERTRKWLEESE |
| 65 | A45L | Belgian | MAVYAVTGGAGFLGRYIVKLLISADDVQEIRVIDIVEDPQPITSKVKVINY |
| | | Outbreak | IQCDINDFDKVREALDGVNLIIHTAALVDVFGKYTDNEIMKVNYYGTQTI |
| | | (Belgium20 | LAACVDLGIKYLIYTSSMEAIGPNKHGNPFIGHEHTLYDISPGHVYAKSK |
| | | 220513) | RMAEQLVMKANNSVIMNGAKLYTCCLRPTGIYGEGDKLTKVFYEQCKQ |
| | | | HGNIMYRTVDDDAVHSRVYVGNVAWMHVLAAKYIQYPGSAIKGNAYF |
| | | | CYDYSPSCSYDMFNLLLMKPLGIEQGSRIPRWMLKMYACKNDMKRILFR |
| | | | KPSILNNYTLKISNTTFEVRTNNAELDFNYSPIFDVDVAFERTRKWLEESE |
| 66 | B9R | Outbreak | MRYIIILAVLFINSIHAKITSYKFESVNFDSKIEWTGDGLYNISLKNYGIKT |
| | | Consensus | WQTMYTNVPEGTYDISGFPKNDFVSFWVKFEQGDYKVEEYCTGLCVEV |
| | | | KIGPPTVILTEYDDHINLFIEHPYATRGSKKIPIYKRGDMCDIYLLYTANFT |
| | | | FGDSEEPVTYDIDDYDCTSTGCSIDFATTEKVCVTAQGATEGFLEKITPWS |
| | | | SEVCLTPKKNVYTCAIRSKEDVPNFKDKIARVITRKFNKQSQSYLTKFLG |
| | | | STSNDVTTFFSILD |
| 67 | B9R | Portuguese | MRYIIILAVLFINSIHAKITSYKFESVNFDSXIEWTGDGLYNISLKNYGIKT |
| | | Outbreak | WQTMYTNVPEGTYDISGFPKNDFVSFWVKFEQGDYKVEEYCTGLCVEV |
| | | (Portugal20 | KIGPPTVILTEYDDHINLFIEHPYATRGSKKIPIYKRGDMCDIYLLYTANFT |
| | | 220504) | FGDSEEPVTYDIDDYDCTSTGCSIDFATTEKVCVTAQGATEGFLEKITPWS |
| | | | SEVCLTPKKNVYTCAIRSKEDVPNFKDKIARVITRKFNKQSQSYLTKFLG |
| | | | STSNDVTTFFSILD |
| 68 | B9R | Belgian | MRYIIILAVLFINSIHAKITSYKFESVNFDSKIEWTGDXLYNISLKNYGIKT |
| | | Outbreak | WQTMYTNVPEGTYDISGFPKNDFVSFWVKFEQGDYKVEEYCTGLCVEV |
| | | (Belgium20 | KIGPPTVILTEYDDHINLFIEHPYATRGSKKIPIYKRGDMCDIYLLYTANFT |
| | | 220513) | FGDSEEPVTYDIDDYDCTSTGCSIDFATTEKVCVTAQGATEGFLEKITPWS |
| | | | SEVCLTPKKNVYTCAIRSKEDVPNFKDKIARVITRKFNKQSQSYLTKFLG |
| | | | STSNDVTTFFSILD |
| 69 | B9R | NC_003310.1 | MRYIIILAVLFINSIHAKITSYKFESVNFDSKIEWTGDGLYNISLKNYGIKT |
| | | | WQTMYTNVPEGTYDISGFPKNDFVSFWVKFEQGDYKVEEYCTGLCVEV |
| | | | KIGPPTVRLTEYDDHINLFIEHPYATRGSKKIPIYKRGDMCDIYLLYTANF |
| | | | TFGDSEEPVTYDIDDYDCTSTGCSIDFATTEKVCVTAQGATEGFLEKITP |
| | | | WSSEVCLTPKKNVYTCAIRSKEDVPNFKDKIARVITRKFNKQSQSYLTKF |
| | | | LGSTSNDVTTFLSILD |
| 70 | B9R | MPXV | MRYIIILAVLFINSIHAKITSYKFESVNFDSKIEWTGDGLYNISLKNYGIKT |
| | | (GenBank: | WQTMYTNVPEGTYDISGFPKNDFVSFWVKFEQGDYKVEEYCTGLCVEV |
| | | MN648051.1) | KIGPPTVILTEYDDHINLFIEHPYATRGSKKIPIYKRGDMCDIYLLYTANFT |
| | | | FGDSEEPVTYDIDDYDCTSTGCSIDFATTEKVCVTAQGATEGFLEKITPWS |
| | | | SEVCLTPKKNVYTCAIRSKEDVPNFKDKIARVITRKFNKQSQSYLTKFLG |
| | | | STSNDVTTFFSILD |
| 71 | B9R | Belgian | MRYIIILAVLFINSIHAKITSYKFESVNFDSKIEWTGDXLYNISLKNYGIKT |
| | | Outbreak | WQTMYTNVPEGTYDISGFPKNDFVSFWVKFEQGDYKVEEYCTGLCVEV |
| | | (Belgium20 | KIGPPTVILTEYDDHINLFIEHPYATRGSKKIPIYKRGDMCDIYLLYTANFT |
| | | 220513) | FGDSEEPVTYDIDDYDCTSTGCSIDFATTEKVCVTAQGATEGFLEKITPWS |
| | | | SEVCLTPKKNVYTCAIRSKEDVPNFKDKIARVITRKFNKQSQSYLTKFLG |
| | | | STSNDVTTFFSILD |
| 72 | B9R | MPXV | MRYIIILAVLFINSIHAKITSYKFESVNFDSKIEWTGDGLYNISLKNYGIKT |
| | | (KJ642617.1) | WQTMYTNVPEGTYDISGFPKNDFVSFWVKFEQGDYKVEEYCTGLCVEV |
| | | | KIGPPTVRLTEYDDHINLFIEHPYATRGSKKIPIYKRGDMCDIYLLYTANF |
| | | | TFGDSEEPVTYDIDDYDCTSTGCSIDFATTEKVCVTAQGATEGFLEKITP |
| | | | WSSEVCLTPKKNVYTCAIRSKEDVPNFKDKIARVITRKFNKQSQSYLTKF |
| | | | LGSTSNDVTTFLSILD |
| 73 | B9R | MPXV | MRYIIILAVLFINSIHAKITSYKFESVNFDSKIEWTGDGLYNISLKNYGIKT |
| | | (KJ642613.1) | WQTMYTNVPEGTYDISGFPKNDFVSFWVKFEQGDYKVEEYCTGLCVEV |
| | | | KIGPPTVRLTEYDDHINLFIEHPYATRGSKKIPIYKRGDMCDIYLLYTANF |
| | | | TFGDSEEPVTYDIDDYDCTSTGCSIDFATTEKVCVTAQGATEGFLEKITP |
| | | | WSSEVCLTPKKNVYTCAIRSKEDVPNFKDKIARVITRKFNKQSQSYLTKF |
| | | | LGSTSNDVTTFLSILD |
| 74 | B9R | MPXV | MRYIIILAVLFINSIHAKITSYKFESVNFDSKIEWTGDGLYNISLKNYGIKT |
| | | (KJ642612.1) | WQTMYTNVPEGTYDISGFPKNDFVSFWVKFEQGDYKVEEYCTGLCVEV |
| | | | KIGPPTVRLTEYDDHINLFIEHPYATRGSKKIPIYKRGDMCDIYLLYTANF |
| | | | TFGDSEEPVTYDIDDYDCTSTGCSIDFATTEKVCVTAQGATEGFLEKITP |
| | | | WSSEVCLTPKKNVYTCAIRSKEDVPNFKDKIARVITRKFNKQSQSYLTKF |
| | | | LGSTSNDVTTFLSILD |
| 75 | B9R | MPXV | MRYIIILAVLFINSIHAKITSYKFESVNFDSKIEWTGDGLYNISLKNYGIKT |
| | | (HM172544.1) | WQTMYTNVPEGTYDISGFPKNDFVSFWVKFEQGDYKVEEYCTGLCVEV |
| | | | KIGPPTVRLTEYDDHINLFIEHPYATRGSKKIPIYKRGDMCDIYLLYTANF |
| | | | TFGDSEEPVTYDIDDYDCTSTGCSIDFATTEKVCVTAQGATEGFLEKITP |
| | | | WSSEVCLTPKKNVYTCAIRSKEDVPNFKDKIARVITRKFNKQSQSYLTKF |
| | | | LGSTSNDVTTFLSILD |
| 76 | B16R | Outbreak | MMKMKMKMKMMVRIYFVSLSLLLFHSYAIDIENEITEFFNKMRDTLPAK |
| | | Consensus | DSKWLNPVCMFGGTMNDMAALGEPFSAKCPPIEDSLLSHRYKDYVVKW |
| | | | ERLEKNRRRQVSNKRVKHGDLWIANYTSKFSNRRYLCTVTTKNGDCVQ |
| | | | GVVRSHVWKPSSCIPKTYELGTYDKYGIDLYCGILYANHYNNITWYKDN |
| | | | KEINIDDFKYSQAGKELIIHNPELEDSGRYDCYVHYDDVRIKNDIVVSRC |
| | | | KILTVIPSQDHRFKLILDPKINVTIGEPANITCSAVSTSLFVDDVLIEWENPS |
| | | | GWIIGLDFGVYSILTSRGGITEATLYFENVTEEYIGNTYTCRGHNYYFDKT |
| | | | LTTTVVLE |
| 77 | B16R | Portuguese | MMKMKMKMKMMVRIYFVSLSLLLFHSYAIDIENEITEFFNKMRDTLPAK |
| | | Outbreak | DSKWLNPVCMFGGTMNDMAALGEPFSAKCPPIEDSLLSHRYKDYVVKW |
| | | (Portugal20 | ERLEKNRRRQVSNKRVKHGDLWIANYTSKFSNRRYLCTVTTKNGDCVQ |
| | | 220504) | GVVRSHVWKPSSCIPKTYELGTYDKYGIDLYCGILYANHYNNITWYKDN |
| | | | KEINIDDFKYSQAGKELIIHNPELEDSGRYDCYVHYDDVRIKNDIVVSRC |
| | | | KILTVIPSQDHRFKLILDPKINVTIGEPANITCSAVSTSLFVDDVLIEWENPS |
| | | | GWIIGLDFGVYSILTSRGGITEATLYFENVTEEYIGNTYTCRGHNYYFDKT |
| | | | LTTTVVLE |
| 78 | B16R | Belgian | MMKMKMKMKMMVRIYFVSLSLLLFHSYAIDIENEITEFFNKMRDTLPAK |
| | | Outbreak | DSKWLNPVCMFGGTMNDMAALGEPFSAKCPPIEDSLLSHRYKDYVVKW |
| | | (Belgium20 | ERLEKNRRRQVSNKRVKHGDLWIANYTSKFSNRRYLCTVTTKNGDCVQ |
| | | 220513) | GVVRSHVWKPSSCIPKTYELGTYDKYGIDLYCGILYANHYNNITWYKDN |
| | | | KEINIDDFKYSQAGKELIIHNPELEDSGRYDCYVHYDDVRIKNDIVVSRC |
| | | | KILTVIPSQDHRFKLILDPKINVTIGEPANITCSAVSTSLFVDDVLIEWENPS |
| | | | GWIIGLDFGVYSILTSRGGITEATLYFENVTEEYIGNTYTCRGHNYYFDKT |
| | | | LTTTVVLE |
| 79 | B16R | NC_003310.1 | MMKMKMMVRIYFVSLSLLLFHSYAIDIENEITEFFNKMRDTLPAKDSKW |
| | | | LNPVCMFGGTMNDMAALGEPFSAKCPPIEDSLLSHRYKDYVVKWERLE |
| | | | KNRRRQVSNKRVKHGDLWIANYTSKFSNRRYLCTVTTKNGDCVQGVVR |
| | | | SHVWKPSSCIPKTYELGTYDKYGIDLYCGILYAKHYNNITWYKDNKEINI |
| | | | DDFKYSQAGKELIIHNPELEDSGRYDCYVHYDDVRIKNDIVVSRCKILTVI |
| | | | PSQDHRFKLILDPKINVTIGEPANITCSAVSTSLFVDDVLIEWENPSGWIIG |
| | | | LDFGVYSILTSRGGITEATLYFENVTEEYIGNTYTCRGHNYYFDKTLTTTV |
| | | | VLE |
| 80 | B16R | MPXV | MMKMKMKMKMMVRIYFVSLSLLLFHSYAIDIENEITEFFNKMRDTLPAK |
| | | (GenBank: | DSKWLNPVCMFGGTMNDMAALGEPFSAKCPPIEDSLLSHRYKDYVVKW |
| | | MN648051.1) | ERLEKNRRRQVSNKRVKHGDLWIANYTSKFSNRRYLCTVTTKNGDCVQ |
| | | | GVVRSHVWKPSSCIPKTYELGTYDKYGIDLYCGILYANHYNNITWYKDN |
| | | | KEINIDDFKYSQAGKELIIHNPELEDSGRYDCYVHYDDVRIKNDIVVSRC |
| | | | KILTVIPSQDHRFKLILDPKINVTIGEPANITCSAVSTSLFVDDVLIEWENPS |
| | | | GWIIGLDFGVYSILTSRGGITEATLYFENVTEEYIGNTYTCRGHNYYFDKT |
| | | | LTTTVVLE |
| 81 | B16R | Belgian | MMKMKMKMKMMVRIYFVSLSLLLFHSYAIDIENEITEFFNKMRDTLPAK |
| | | Outbreak | DSKWLNPVCMFGGTMNDMAALGEPFSAKCPPIEDSLLSHRYKDYVVKW |
| | | (Belgium20 | ERLEKNRRRQVSNKRVKHGDLWIANYTSKFSNRRYLCTVTTKNGDCVQ |
| | | 220513) | GVVRSHVWKPSSCIPKTYELGTYDKYGIDLYCGILYANHYNNITWYKDN |
| | | | KEINIDDFKYSQAGKELIIHNPELEDSGRYDCYVHYDDVRIKNDIVVSRC |
| | | | KILTVIPSQDHRFKLILDPKINVTIGEPANITCSAVSTSLFVDDVLIEWENPS |
| | | | GWIIGLDFGVYSILTSRGGITEATLYFENVTEEYIGNTYTCRGHNYYFDKT |
| | | | LTTTVVLE |
| 82 | B16R | MPXV | MKMKMMVRIYFVSLSLLLFHSYAIDIENEITEFFNKMRDTLPAKDSKWL |
| | | (KJ642617.1) | NPVCMFGGTMNDMAALGEPFSAKCPPIEDSLLSHRYKDYVVKWERLEK |
| | | | NRRRQVSNKRVKHGDLWIANYTSKFSNRRYLCTVTTKNGDCVQGVVRS |
| | | | HVWKPSSCIPKTYELGTYDKYGIDLYCGILYANHYNNITWYKDNKEINID |
| | | | DFKYSQAGKELIIHNPELEDSGRYDCYVHYDDVRIKNDIVVSRCKILTVIP |
| | | | SQDHRFKLILDPKINVTIGEPANITCSAVSTSLFVDDVLIEWENPSGWIIGL |
| | | | DFGVYSILTSRGGITEATLYFENVTEEYIGNTYTCRGHNYYFDKTLTTTV |
| | | | VLE |
| 83 | B16R | MPXV | MMKMKMMVRIYFVSLSLLLFHSYAIDIENEITEFFNKMRDTLPAKDSKW |
| | | (KJ642613.1) | LNPVCMFGGTMNDMAALGEPFSAKCPPIEDSLLSHRYKDYVVKWERLE |
| | | | KNRRRQVSNKRVKHGDLWIANYTSKFSNRRYLCTVTTKNGDCVQGVVR |
| | | | SHVWKPSSCIPKTYELGTYDKYGIDLYCGILYAKHYNNITWYKDNKEINI |
| | | | DDFKYSQAGKELIIHNPELEDSGRYDCYVHYDDVRIKNDIVVSRCKILTVI |
| | | | PSQDHRFKLILDPKINVTIGEPANITCSAVSTSLFVDDVLIEWENPSGWIIG |
| | | | LDFGVYSILTSRGGITEATLYFENVTEEYIGNTYTCRGHNYYFDKTLTTTV |
| | | | VLE |
| 84 | B16R | MPXV | MMKMKMMVRIYFVSLSLLLFHSYAIDIENEITEFFNKMRDTLPAKDSKW |
| | | (KJ642612.1) | LNPVCMFGGTMNDMAALGEPFSAKCPPIEDSLLSHRYKDYVVKWERLE |
| | | | KNRRRQVSNKRVKHGDLWIANYTSKFSNRRYLCTVTTKNGDCVQGVVR |
| | | | SHVWKPSSCIPKTYELGTYDKYGIDLYCGILYAKHYNNITWYKDNKEINI |
| | | | DDFKYSQAGKELIIHNPELEDSGRYDCYVHYDDVRIKNDIVVSRCKILTVI |
| | | | PSQDHRFKLILDPKINVTIGEPANITCSAVSTSLFVDDVLIEWENPSGWIIG |
| | | | LDFGVYSILTSRGGITEATLYFENVTEEYIGNTYTCRGHNYYFDKTLTTTV |
| | | | VLE |
| 85 | B16R | MPXV | MMKMKMMVRIYFVSLSLLLFHSYAIDIENEITEFFNKMRDTLPAKDSKW |
| | | (HM172544.1) | LNPVCMFGGTMNDMAALGEPFSAKCPPIEDSLLSHRYKDYVVKWERLE |
| | | | KNRRRQVSNKRVKHGDLWIANYTSKFSNRRYLCTVTTKNGDCVQGVVR |
| | | | SHVWKPSSCIPKTYELGTYDKYGIDLYCGILYAKHYNNITWYKDNKEINI |
| | | | DDFKYSQAGKELIIHNPELEDSGRYDCYVHYDDVRIKNDIVVSRCKILTVI |
| | | | PSQDHRFKLILDPKINVTIGEPANITCSAVSTSLFVDDVLIEWENPSGWIIG |
| | | | LDFGVYSILTSRGGITEATLYFENVTEEYIGNTYTCRGHNYYFDKTLTTTV |
| | | | VLE |
| 86 | C10L | Outbreak | MEPILAPNPNRFVIFPIQYHDIWNMYKKAEASFWTVEEVDISKDINDWNK |
| | | Consensus | LTPDEKYFIKHVLAFFAASDGIVNENLAERFCTEVQITEARCFYGFQMAIE |
| | | | NIHSEMYSLLIDTYVKDSNEKNYLFNAIETMPCVKKKADWAQKWIHDSA |
| | | | GYGERLIAFAAVEGIFFSGSFASIFWLKKRGLMPGLTFSNELISRDEGLHC |
| | | | DFACLMFKHLLHPPSEETVRFIITNAVSIEQEFLTVALPVKLIGMNCEMM |
| | | | KTYIEFVADRLISELGFKKIYNVTNPFDFMENISLEGKTNFFEKRVGEYQK |
| | | | MGVMSQEDNHFSLDVDF |
| 87 | C10L | Portuguese | MEPILAPNPNRFVIFPIQYHDIWNMYKXAEXSFWTVEXVDISKDINDWNK |
| | | Outbreak | LTPDEKYFIKHVLAFFAASDGIVNENLAERFCTEVQITEARCFYGFQMAIE |
| | | (Portugal20 | NIHSEMYSLLIDTYVKDSNEKNYLFNAIETMPCVKXKADWAQKWIHDSA |
| | | 220504) | GYGERLIAFAAVEGIFFXGSFASIFWLKXRGLMPGLTFSNELISRDEGLHC |
| | | | DFACLMFKHLLHPPSEETVRFIITNAVSIEQEFLTVALPVKLIGMNCEMM |
| | | | KTYIEFVADRLISELGFKKXYNVTNPFDFMENISLEGKTNFXEKRVGEYQ |
| | | | KMGVMSQEDNHFSLDVDF |
| 88 | C10L | Belgian | MEPILAPNPNRFVIFPIQYHDIWNMYKKAEASFWTVEEVDISKDINDWNK |
| | | Outbreak | LTPDEKYFIKHVLAFFAASDGIVNENLAERFCTEVQITEARCFYGFQMAIE |
| | | (Belgium20 | NIHSEMYSLLIDTYVKDSNEKNYLFNAIETMPCVKKKADWAQKWIHDSA |
| | | 220513) | GYGERLIAFAAVEGIFFSGSFASIFWLKKRGLMPGLTFSNELISRDEGLHC |
| | | | DFACLMFKHLLHPPSEETVRFIITNAVSIEQEFLTVALPVKLIGMNCEMM |
| | | | KTYIEFVADRLISELGFKKIYNVTNPFDFMENISLEGKTNFFEKRVGEYQK |
| | | | MGVMSQEDNHFSLDVDF |
| 89 | C10L | NC_003310.1 | MEPILAPNPNRFVIFPIQYHDIWNMYKKAEASFWTVEEVDISKDINDWNK |
| | | | LTPDEKYFIKHVLAFFAASDGIVNENLAERFCTEVQITEARCFYGFQMAIE |
| | | | NIHSEMYSLLIDTYVKDSNEKNYLFNAIETMPCVKKKADWAQKWIHDSA |
| | | | GYGERLIAFAAVEGIFFSGSFASIFWLKKRGLMPGLTFSNELISRDEGLHC |
| | | | DFACLMFKHLLHPPSEETVRFIITNAVSIEQEFLTVALPVKLIGMNCEMM |
| | | | KTYIEFVADRLISELGFKKIYNVTNPFDFMENISLEGKTNFFEKRVGEYQK |
| | | | MGVMSQEDNHFSLDVDF |
| 90 | C10L | Belgian | MEPILAPNPNRFVIFPIQYHDIWNMYKKAEASFWTVEEVDISKDINDWNK |
| | | Outbreak | LTPDEKYFIKHVLAFFAASDGIVNENLAERFCTEVQITEARCFYGFQMAIE |
| | | (Belgium20 | NIHSEMYSLLIDTYVKDSNEKNYLFNAIETMPCVKKKADWAQKWIHDSA |
| | | 220513) | GYGERLIAFAAVEGIFFSGSFASIFWLKKRGLMPGLTFSNELISRDEGLHC |
| | | | DFACLMFKHLLHPPSEETVRFIITNAVSIEQEFLTVALPVKLIGMNCEMM |
| | | | KTYIEFVADRLISELGFKKIYNVTNPFDFMENISLEGKTNFFEKRVGEYQK |
| | | | MGVMSQEDNHFSLDVDF |
| 91 | C10L | MA | MEPILAPNPNRFVIFPIQYHDIWNMYKKAEASFWTVEEVDISKDINDWNK |
| | | Outbreak | LTPDEKYFIKHVLAFFAASDGIVNENLAERFCTEVQITEARCFYGFQMAIE |
| | | | NIHSEMYSLLIDTYVKDSNEKNYLFNAIETMPCVKKKADWAQKWIHDSA |
| | | | GYGERLIAFAAVEGIFFSGSFASIFWLKKAWPNARTHVF |
| 92 | C21L | Outbreak | MRSIAGLDKLKMEIFNVEELINMKPFKNMNKITINQKDNCILANRCFVKI |
| | | Consensus | DTPRYIPSTSISSSNIIRIRNHDFTLSELLYSPFHFQQPQFQYLLPGFVLTCID |
| | | | KVSKQQKECKYCISNRGDDDSLSINIFIPTINKSIYIIIGLRMKNFWKPKFEI |
| | | | E |
| 93 | C21L | Portuguese | MRSIAGLDKLKMEIFNVEELINMKPFKNMNKITINQKDNCILANRCFVKI |
| | | Outbreak | DTPRYIPSTSISSSNIIRIRNHDFTLSELLYSPFHFQQPQFQYLLPGFVLTCID |
| | | (Portugal20 | KVSKQQKECKYCISNRGDDDSLSINIFIPTINKSIYIIIGLRMKNFWKPKFEI |
| | | 220504) | E |
| 94 | C21L | Belgian | MRSIAGLDKLKMEIFNVEELINMKPFKNMNKITINQKDNCILANRCFVKI |
| | | Outbreak | DTPRYIPSTSISSSNIIRIRNHDFTLSELLYSPFHFQQPQFQYLLPGFVLTCID |
| | | (Belgium20 | KVSKQQKECKYCISNRGDDDSLSINIFIPTINKSIYIIIGLRMKNFWKPKFEI |
| | | 220513) | E |
| 95 | C21L | NC_003310.1 | MRSIAGLDKLKMEIFNVEELINMKPFKNMNKITINQKDNCILANRCFVKI |
| | | | DTPRYIPSTSISSSNIIRIRNHDFTLSELLYSPFHFQQPQFQYLLPGFVLTCID |
| | | | KVSKQQKECKYCISNRGDDDSLSINIFIPTINKSIYIIIGLRMKNFWKPKFEI |
| | | | E |
| 96 | C21L | Belgian | MRSIAGLDKLKMEIFNVEELINMKPFKNMNKITINQKDNCILANRCFVKI |
| | | Outbreak | DTPRYIPSTSISSSNIIRIRNHDFTLSELLYSPFHFQQPQFQYLLPGFVLTCID |
| | | (Belgium20 | KVSKQQKECKYCISNRGDDDSLSINIFIPTINKSIYIIIGLRMKNFWKPKFEI |
| | | 220513) | E |
| 97 | E7R | Outbreak | MSSFVTNGYLPVTLEPHELTLDIKTNIRNAVYKAYLHREISGKMAKKIEIR |
| | | Consensus | EDVELPLGEIVNNSVVINVPCVITYAYYHVGDIVRGTLNIEDESNVTIQCG |
| | | | DLICKLSRDSGTVSFSDSKYCFFRNGNAYDNGIEVSAVLMEAQQGTESSF |
| | | | VFLANIVDS |
| 98 | E7R | Portuguese | MSSXVTNGYLPVTXEXHEXTLDXKTNIXNAVYKAYXHXXXXGXMAKX |
| | | Outbreak | IEIREDVELXXGEIVNNSVVINVPXXXTYAYYHVXDIVRGTXNIEDESNV |
| | | (Portugal20 | TIXXXDLXCKLSRDSGTVSFSDSXYXXFRNXXAYDNGIEVSAVLMEAQQ |
| | | 220504) | GTESSFVFXANIVDX |
| 99 | E7R | Belgian | MSSFVTNGYLPVTLEPHELTLDIKTNIRNAVYKAYLHREISGKMAKKIEIR |
| | | Outbreak | EDVELPLGEIVNNSVVINVPCVITYAYYHVGDIVRGTLNIEDESNVTIQCG |
| | | (Belgium20 | DLICKLSRDSGTVSFSDSKYCFFRNGNAYDNGIEVSAVLMEAQQGTESSF |
| | | 220513) | VFLANIVDS |
| 100 | E7R | NC_003310.1 | MSSFVTNGYLPVTLEPHELTLDIKTNIRNAVYKAYLHREISGKMAKKIEIR |
| | | | EDVELPLGEIVNNSVVINVPCVITYAYYHVGDIVRGTLNIEDESNVTIQCG |
| | | | DLICKLSRDSGTVSFSDSKYCFFRNGNAYDNGIEVSAVLMEAQQGTESSF |
| | | | VFLANIVDS |
| 101 | E7R | Belgian | MSSFVTNGYLPVTLEPHELTLDIKTNIRNAVYKAYLHREISGKMAKKIEIR |
| | | Outbreak | EDVELPLGEIVNNSVVINVPCVITYAYYHVGDIVRGTLNIEDESNVTIQCG |
| | | (Belgium20 | DLICKLSRDSGTVSFSDSKYCFFRNGNAYDNGIEVSAVLMEAQQGTESSF |
| | | 220513) | VFLANIVDS |
| 102 | F3L | Outbreak | MEKREVNKALYDLQRSAMVYSSNDTPPRWSTTMDADTRPTDSDADAII |
| | | Consensus | DDVSREKSMREDHKSFDDVIPVKKIIYWKGVNPVTVINEYCQITRRDWSF |
| | | | RIESVGPSNSPTFYACVDIDGRVFDKADGKSKRDAKNNAAKLAVDKLLS |
| | | | YVIIRF |
| 103 | F3L | Portuguese | MEKREVNKALYDLQRSAMVYSSNDTPPRWSTTMDADTRPTDSDADAII |
| | | Outbreak | DDVSREKSMREDHKSFDDVIPVKKIIYWKGVNPVTVINEYCQITRRDWSF |
| | | (Portugal20 | RIESVGPSNSPTFYACVDIDGRVFDKADGKSKRDAKNNAAKLAVDKLLS |
| | | 220504) | YVIIRF |
| 104 | F3L | Belgian | MEKREVNKALYDLQRSAMVYSSNDTPPRWSTTMDADTRPTDSDADAII |
| | | Outbreak | DDVSREKSMREDHKSFDDVIPVKKIIYWKGVNPVTVINEYCQITRRDWSF |
| | | (Belgium20 | RIESVGPSNSPTFYACVDIDGRVFDKADGKSKRDAKNNAAKLAVDKLLS |
| | | 220513) | YVIIRF |
| 105 | F3L | NC_003310.1 | MEKREVNKALYDLQRSTMVYSSDDTPPRWSTTMDADTRPTDSDADAIID |
| | | | DVSREKSMREDNKSFDDVIPVKKIIYWKGVNPVTVINEYCQITRRDWSFR |
| | | | IESVGPSNSPTFYACVDIDGRVFDKADGKSKRDAKNNAAKLAVDKLLSY |
| | | | VIIRF |
| 106 | F3L | MPXV | MEKREVNKALYDLQRSAMVYSSNDTPPRWSTTMDADTRPTDSDADAII |
| | | (GenBank: | DDVSREKSMREDHKSFDDVIPVKKIIYWKGVNPVTVINEYCQITRRDWSF |
| | | MN648051.1) | RIESVGPSNSPTFYACVDIDGRVFDKADGKSKRDAKNNAAKLAVDKLLS |
| | | | YVIIRF |
| 107 | F3L | Belgian | MEKREVNKALYDLQRSAMVYSSNDTPPRWSTTMDADTRPTDSDADAII |
| | | Outbreak | DDVSREKSMREDHKSFDDVIPVKKIIYWKGVNPVTVINEYCQITRRDWSF |
| | | (Belgium20 | RIESVGPSNSPTFYACVDIDGRVFDKADGKSKRDAKNNAAKLAVDKLLS |
| | | 220513) | YVIIRF |
| 108 | F3L | MPXV | MEKREVNKALYDLQRSAMVYSSNDTPPRWSTTMDADTRPTDSDADAII |
| | | (KJ642617.1) | DDVSREKSMREDHKSFDDVIPVKKIIYWKGVNPVTVINEYCQITRRDWSF |
| | | | RIESVGPSNSPTFYACVDIDGRVFDKADGKSKRDAKNNAAKLAVDKLLS |
| | | | YVIIRF |
| 109 | F3L | MPXV | MEKREVNKALYDLQRSTMVYSSDDTPPRWSTTMDADTRPTDSDADAIID |
| | | (KJ642613.1) | DVSREKSMREDNKSFDDVIPVKKIIYWKGVNPVTVINEYCQITRRDWSFR |
| | | | IESVGPSNSPTFYACVDIDGRVFDKADGKSKRDAKNNAAKLAVDKLLSY |
| | | | VIIRF |
| 110 | F3L | MPXV | MEKREVNKALYDLQRSTMVYSSDDTPPRWSTTMDADTRPTDSDADAIID |
| | | (KJ642612.1) | DVSREKSMREDNKSFDDVIPVKKIIYWKGVNPVTVINEYCQITRRDWSFR |
| | | | IESVGPSNSPTFYACVDIDGRVFDKADGKSKRDAKNNAAKLAVDKLLSY |
| | | | VIIRF |
| 111 | F3L | MPXV | MEKREVNKALYDLQRSTMVYSSDDTPPRWSTTMDADTRPTDSDADAIID |
| | | (HM172544.1) | DVSREKSMREDNKSFDDVIPVKKIIYWKGVNPVTVINEYCQITRRDWSFR |
| | | | IESVGPSNSPTFYACVDIDGRVFDKADGKSKRDAKNNAAKLAVDKLLSY |
| | | | VIIRF |
| 112 | F4L | Outbreak | MENVYISSYSSNEQISMAVVTTDIRELLSQYVDDANLENLIEWAMEKSSK |
| | | Consensus | YYIKNIGNTKSNIEETKFESKNNIGIEYSKDSKNKLSYRNKPSIATHLEYKT |
| | | | LCDMIKGTSGTEKEFLRYLLFGIKCIKKGVEYNIDKIKDVSYNDYFNVLD |
| | | | EKYNTPCPNCKSRNTTPMMIQTRAADEPPLVRHACRDCKQHFKPPKFRA |
| | | | FRNLNVTTQSIHENKEITEILPDNNPSPPESPEPASPIDDGLIRATFDRNDEP |
| | | | PEDDE |
| 113 | F4L | Portuguese | MENVYISSYSSNEQISMAVVTTDIRELLSQYVDDANLENLIEWAMEKSSK |
| | | Outbreak | YYIKNIGNTKSNIEETKFESKNNIGIEYSKDSKNKLSYRNKPSIATHLEYKT |
| | | (Portugal20 | LCDMIKGTSGTEKEFLRYLLFGIKCIKKGVEYNIDKIKDVSYNDYFNVLD |
| | | 220504) | EKYNTPCPNCKSRNTTPMMIQTRAADEPPLVRHACRDCKQHFKPPKFRA |
| | | | FRNLNVTTQSIHENKEITEILPDNNPSPPESPEPASPIDDGLIRATFDRNDEP |
| | | | PEDDE |
| 114 | F4L | Belgian | MENVYISSYSSNEQISMAVVTTDIRELLSQYVDDANLENLIEWAMEKSSK |
| | | Outbreak | YYIKNIGNTKSNIEETKFESKNNIGIEYSKDSKNKLSYRNKPSIATHLEYKT |
| | | (Belgium20 | LCDMIKGTSGTEKEFLRYLLFGIKCIKKGVEYNIDKIKDVSYNDYFNVLD |
| | | 220513) | EKYNTPCPNCKSRNTTPMMIQTRAADEPPLVRHACRDCKQHFKPPKFRA |
| | | | FRNLNVTTQSIHENKEITEILPDNNPSPPESPEPASPIDDGLIRATFDRNDEP |
| | | | PEDDE |
| 115 | F4L | NC_003310.1 | MENVYISSYSSNEQISMAVVTTDIRELLSQYVDDANLENLIEWAMEKSSK |
| | | | YYIKNIGNTKSNIEETKFESKNNIGIEYSKDSRNKLSYRNKPSIATNLEYKT |
| | | | LCDMIKGTSGTEKEFLRYLLFGIKCIKKGVEYNIDKIKDVSYNDYFNVLD |
| | | | EKYNTPCPNCKSRNTTPMMIQTRAADEPPLVRHACRDCKQHFKPPKFRA |
| | | | FRNLNVTTQSIHENKEITEILPDNNPSPPESPEPASPIDDGLIRATFDRNDEP |
| | | | PEDDE |
| 116 | F4L | Belgian | MENVYISSYSSNEQISMAVVTTDIRELLSQYVDDANLENLIEWAMEKSSK |
| | | Outbreak | YYIKNIGNTKSNIEETKFESKNNIGIEYSKDSKNKLSYRNKPSIATHLEYKT |
| | | (Belgium20 | LCDMIKGTSGTEKEFLRYLLFGIKCIKKGVEYNIDKIKDVSYNDYFNVLD |
| | | 220513) | EKYNTPCPNCKSRNTTPMMIQTRAADEPPLVRHACRDCKQHFKPPKFRA |
| | | | FRNLNVTTQSIHENKEITEILPDNNPSPPESPEPASPIDDGLIRATFDRNDEP |
| | | | PEDDE |
| 117 | G6R | Outbreak | MVFQLVCSTCGKDISHERYKLIIRKKSLKDVLVSVKNECCRLKLSTQIEPQ |
| | | Consensus | RNLTVQPLLDIN |
| 118 | G6R | Portuguese | MVFQLVCSTCGKDISHERYKLIIRKKSLKDVLVSVKNECCRLKLSTQIEPQ |
| | | Outbreak | RNLTVQPLXDIN |
| | | (Portugal20 | |
| | | 220504) | |
| 119 | G6R | Belgian | MVFQLVCSTCGKDISHERYKLIIRKKSLKDVLVSVKNECCRLKLSTQIEPQ |
| | | Outbreak | RNLTVQPLLDIN |
| | | (Belgium20 | |
| | | 220513) | |
| 120 | G6R | NC_003310.1 | MVFQLVCSTCGKDISHERYKLIIRKKSLKDVLVSVKNECCRLKLSTQIEPQ |
| | | | RNLTVQPLLDIN |
| 121 | G6R | Belgian | MVFQLVCSTCGKDISHERYKLIIRKKSLKDVLVSVKNECCRLKLSTQIEPQ |
| | | Outbreak | RNLTVQPLLDIN |
| | | (Belgium20 | |
| | | 220513) | |
| 122 | H5R | Outbreak | MAWSITNKADTSSFTKMAEIRAHLRNSAENKDKNEDIFPEDVIIPSTKPKT |
| | | Consensus | KRTTTPRKPAATKRSTKKDKEKEEVEEVVIEEYHQTTEENSPPPSSSPGVG |
| | | | DIVESVAAVELDDSDGDDEPMVQVEAGKVNHSARSDLSDLKVATDNIV |
| | | | KDLKKIITRISAVSTVLEDVQAAGISRQFTSMTKAITTLSDLVTEGKSKVV |
| | | | RKKVKTCKK |
| 123 | H5R | Portuguese | MAWSITNKADTSSFTKMAEIRAHLRNSAENKDKNEDIFPEDVIIPSTKPKT |
| | | Outbreak | KRTTTPRKPAATKRSTKKDKEKEEVEEVVIEEYHQTTEENSPPPSSSPGVG |
| | | (Portugal20 | DIVESVAAVELDDSDGDDEPMVQVEAGKVNHSARSDLSDLKVATDNIV |
| | | 220504) | KDLKKIITRISAVSTVLEDVQAAGISRQFTSMTKAITTLSDLVTEGKSKVV |
| | | | RKKVKTCKK |
| 124 | H5R | Belgian | MAWSITNKADTSSFTKMAEIRAHLRNSAENKDKNEDIFPEDVIIPSTKPKT |
| | | Outbreak | KRTTTPRKPAATKRSTKKDKEKEEVEEVVIEEYHQTTEENSPPPSSSPGVG |
| | | (Belgium20 | DIVESVAAVELDDSDGDDEPMVQVEAGKVNHSARSDLSDLKVATDNIV |
| | | 220513) | KDLKKIITRISAVSTVLEDVQAAGISRQFTSMTKAITTLSDLVTEGKSKVV |
| | | | RKKVKTCKK |
| 125 | H5R | NC_003310.1 | MAWSITNKADTSSFTKMAEIRAHLRNSAENKDKNEDIFPEDVIIPSTKPKT |
| | | | KRTTTPRKPAATKRSTKKDKEKEEVEEVEEVVIEEYHQTTEENSPPPSSSP |
| | | | GVGDIVESVAAVELDDSDGDDEPMVQVEAGKVNHSARSDLSDLKVATD |
| | | | NIVKDLKKIITRISAVSTVLEDVQAAGISRQFTSMTKAITTLSDLVTEGKSK |
| | | | VVRKKVKTCKK |
| 126 | H5R | Belgian | MAWSITNKADTSSFTKMAEIRAHLRNSAENKDKNEDIFPEDVIIPSTKPKT |
| | | Outbreak | KRTTTPRKPAATKRSTKKDKEKEEVEEVVIEEYHQTTEENSPPPSSSPGVG |
| | | (Belgium20 | DIVESVAAVELDDSDGDDEPMVQVEAGKVNHSARSDLSDLKVATDNIV |
| | | 220513) | KDLKKIITRISAVSTVLEDVQAAGISRQFTSMTKAITTLSDLVTEGKSKVV |
| | | | RKKVKTCKK |
| 127 | I3L | Outbreak | MSKVIKKRVETSPRPTASSDSLQTCAGVIEYAKSISKSNAKCIECVTLNAS |
| | | Consensus | QYANCSSISIKLTDSLSSQMTSTFIMLEGETKLYKNKSKQDRSDGYFLKIK |
| | | | VTAASPMLYQLLEAVYGNITHKERIPNSLHSLSAETITEKTFKDESIFINKL |
| | | | NGAMVEYVSTGESSILRSIEGELESLSKRERQLAKAIITPVVFYRSGTETKI |
| | | | TFALKKLIIDREVVANVIGLSGDSERVSMTENVEEDLVRNLGLVDIDDEY |
| | | | DEDSDKEKPIFNV |
| 128 | I3L | Portuguese | MSKVIKKRVETSPRPTASSDSLQTCAGVIEYAKSISKSNAXCIECVTLNAS |
| | | Outbreak | QYANCSSISIKLTDSLSSQMTSTFIMLEGETKLYKNKSKQDRSDGYFXKIK |
| | | (Portugal20 | VTAASPMLYQLLEAVYGNITHKERIPNSLHSLSAETITEKTFKDESIFINKL |
| | | 220504) | NGAMVEYVSTGESSILRSIEGELESLSKRERQLAKAIITPVVFYRSGTETKI |
| | | | TFALKKLIIDREVVANVIGLSGDSERVSMTENVEEDLVRNLGLVDIDDEY |
| | | | DEDSDKEKPIFNV |
| 129 | I3L | Belgian | MSKVIKKRVETSPRPTASSDSLQTCAGVIEYAKSISKSNAKCIECVTLNAS |
| | | Outbreak | QYANCSSISIKLTDSLSSQMTSTFIMLEGETKLYKNKSKQDRSDGYFLKIK |
| | | (Belgium20 | VTAASPMLYQLLEAVYGNITHKERIPNSLHSLSAETITEKTFKDESIFINKL |
| | | 220513) | NGAMVEYVSTGESSILRSIEGELESLSKRERQLAKAIITPVVFYRSGTETKI |
| | | | TFALKKLIIDREVVANVIGLSGDSERVSMTENVEEDLVRNLGLVDIDDEY |
| | | | DEDSDKEKPIFNV |
| 130 | I3L | NC_003310.1 | MSKVIKKRVETSPRPTASSDSLQTCAGVIEYAKSISKSNAKCIEYVTLNAS |
| | | | QYANCSSISIKLTDSLSSQMTSTFIMLEGETKLYKNKSKQDRSDGYFLKIK |
| | | | VTAASPMLYQLLEAVYGNITHKERIPNSLHSLSAETITEKTFKDESIFINKL |
| | | | NGAMVEYVSTGESSILRSIEGELESLSKRERQLAKAIITPVVFYRSGTETKI |
| | | | TFALKKLIIDREVVANVIGLSGDSERVSMTENVEEDLVRNLGLVDIDDEY |
| | | | DEDSDKEKPIFNV |
| 131 | I3L | Belgian | MSKVIKKRVETSPRPTASSDSLQTCAGVIEYAKSISKSNAKCIECVTLNAS |
| | | Outbreak | QYANCSSISIKLTDSLSSQMTSTFIMLEGETKLYKNKSKQDRSDGYFLKIK |
| | | (Belgium20 | VTAASPMLYQLLEAVYGNITHKERIPNSLHSLSAETITEKTFKDESIFINKL |
| | | 220513) | NGAMVEYVSTGESSILRSIEGELESLSKRERQLAKAIITPVVFYRSGTETKI |
| | | | TFALKKLIIDREVVANVIGLSGDSERVSMTENVEEDLVRNLGLVDIDDEY |
| | | | DEDSDKEKPIFNV |
| 132 | O2L | Outbreak | MVYKLVLLFCIASLGYSVEYKNTICPHRQDYRYWYFVAELTIGVNYDIN |
| | | Consensus | STIIGECHMSESYIDRNANIVLTGYGLKVNMTIMDTDQRFVAAAEGVGK |
| | | | DNKLSVLLFTTQRLDKVHHNISVTITCMEMNCGTTKYNSDLPESIHKSSS |
| | | | CDITINGSCVTCVNLETDPTKINPHYLHPKNKYLYHNSEYSMRGSYGVTF |
| | | | IDELNQCLLDIKELSYDICYRE |
| 133 | O2L | Portuguese | MVYKLVLLFCIASLGYSVEYKNTICPHRQDYRYWYFVAELTIGVNYDIN |
| | | Outbreak | STIIGECHMSESYIDRNANIVLTGYGLKVNMTIMDTDQRFVAAAEGVGK |
| | | (Portugal20 | DNKLSVLLFTTQRLDKVHHNISVTITCMEMNCGTTKYNSDLPESIHKSSS |
| | | 220504) | CDITINGSCVTCVNLETDPTKINPHYLHPKNKYLYHNSEYSMRGSYGVTF |
| | | | IDELNQCLLDIKELSYDICYRE |
| 134 | O2L | Belgian | MVYKLVLLFCIASLGYSVEYKNTICPHRQDYRYWYFVAELTIGVNYDIN |
| | | Outbreak | STIIGECHMSESYIDRNANIVLTGYGLKVNMTIMDTDQRFVAAAEGVGK |
| | | (Belgium20 | DNKLSVLLFTTQRLDKVHHNISVTITCMEMNCGTTKYNSDLPESIHKSSS |
| | | 220513) | CDITINGSCVTCVNLETDPTKINPHYLHPKNKYLYHNSEYSMRGSYGVTF |
| | | | IDELNQCLLDIKELSYDICYRE |
| 135 | O2L | NC_003310.1 | MVYKLVLLFCIASLGYSVEYKNTICPPRQDYRYWYFVAELTIGVNYDINS |
| | | | TIIGECHMSESYIDRNANIVLTGYGLKINMTIMDTDQRFVAAAEGVGKDN |
| | | | KLSVLLFTTQRLDKVHHNISVTITCMEMNCGTTKYNSDLPESIHKSSSCDI |
| | | | TINGSCVTCVNLETDPTKINPHYLHPKNKYLYHNSEYSMRGSYGVTFIDE |
| | | | LNQCLLDIKELSYDICYRE |
| 136 | O2L | Belgian | MVYKLVLLFCIASLGYSVEYKNTICPHRQDYRYWYFVAELTIGVNYDIN |
| | | Outbreak | STIIGECHMSESYIDRNANIVLTGYGLKVNMTIMDTDQRFVAAAEGVGK |
| | | (Belgium20 | DNKLSVLLFTTQRLDKVHHNISVTITCMEMNCGTTKYNSDLPESIHKSSS |
| | | 220513) | CDITINGSCVTCVNLETDPTKINPHYLHPKNKYLYHNSEYSMRGSYGVTF |
| | | | IDELNQCLLDIKELSYDICYRE |
| 137 | Q1L | Outbreak | MFMYPEFARKALSKLISKKLNIEKVSSKHQLVLLDYGLHGLLPKSLYLEA |
| | | Consensus | INSDILNVRFFPPEIINVTDIVKALQNSCRVDEYLKAVSLYHKNSLMVSGP |
| | | | NVVKLMIEYNLLTHSDLEWLINENVVKATYLLKINAYMINFKIDLTVDEII |
| | | | DLVKDIPVGATLHLYNILNNIDLDIILRISDEYNIPPVHDILSKLTNEEMCIK |
| | | | LVTKYPMDNVINFINQDVRYSPTFIKTIKDFVNEHLPTMYDGLNDYLHSV |
| | | | IIDEDLIEEYKIKSVAMFNLEYKTDIDTLTLDEQIFVEVNISYYDFRYRQFA |
| | | | DEFRDYIMIKERRQITMQSGDRIRRFRRPMSLRSTIIKKDTDSLEDILSHID |
| | | | NAKKNSKVSIEDVERIISSFRLNPCVVRRTMLSDIDIKTKIMVLKIAKDWK |
| | | | SCTLTLSVKGIMVTDTINTVLSKILHHHRNIFKYLTSVENKEIAVCNCSRC |
| | | | LSLFYRKLKSVRCDLRTDDGLLDRLYDLTRYALHGKINQNLIGQRCWGP |
| | | | LTEILFNENKKKKLNNLMEYIKISDMLVYGHYIEKTLIPITDSLSFKLFVDT |
| | | | MSVLNDQYAKVVIFFNTIIEYIIATIYYRLTVLDNYTNVKHFVSKVLHTV |
| | | | MEACGVLFSHIKVNDKIEYELEEMVDKGTVPSYLYHLSINVISIILDDING |
| | | | TR |
| 138 | Q1L | Portuguese | MFMYPEFARKALSKLISKKLNIEKVSSKHQLVLLDYGLHGLLPKSLYLEA |
| | | Outbreak | INSDILNVRFFPPEIINVTDIVKALQNSCRVDEYLKAVSLYHKNSLMVSGP |
| | | (Portugal20 | NVVKLMIEYNLLTHSDLEWLINENVVKATYLLKINAYMINFKIDLTVDEII |
| | | 220504) | DLVKDIPVGATLHLYNILNNIDLDIILRISDEYNIPPVHDILSKLTNEEMCIK |
| | | | LVTKYPMDNVINFINQDVRYSPTFIKTIKDFVNEHLPTMYDGLNDYLHSV |
| | | | IIDEDLIEEYKIKSVAMFNLEYKTDIDTLTLDEQIFVEVNISYYDFRYRQFA |
| | | | DEFRDYIMIKERRQITMQSGDRIRRFRRPMSLRSTIIKKDTDSLEDILSHID |
| | | | NAKKXSKVSIEDVERIISSFRLNPCVVRRTMLSDIDIKTKIMVLKIAKXWK |
| | | | SCTLTLSVKGIMVTDTINTVLSKILHHHRNIFKYLTSVENKEIAVCNCSRC |
| | | | LSLFYRKLKSVRCDLRTDDGLLDRLYDLTRYALHGKINQNLIGQRCWGP |
| | | | LTEILFNENKKKXLNNLMEYIKISDMLVYGHYIEKTLIPITDSLSFKLFVDT |
| | | | MSVLNDQYAKVVIFFNTIIEYIIATIYYRLTVLDNYTNVKHFVSKVLHTV |
| | | | MEACGVLFSHIKVNDKIEYELEEMVDKGTVPSYLYHLSINVISIILDDING |
| | | | TR |
| 139 | Q1L | Belgian | MFMYPEFARKALSKLISKKLNIEKVSSKHQLVLLDYGLHGLLPKSLYLEA |
| | | Outbreak | INSDILNVRFFPPEIINVTDIVKALQNSCRVDEYLKAVSLYHKNSLMVSGP |
| | | (Belgium20 | NVVKLMIEYNLLTHSDLEWLINENVVKATYLLKINAYMINFKIDLTVDEII |
| | | 220513) | DLVKDIPVGATLHLYNILNNIDLDIILRISDEYNIPPVHDILSKLTNEEMCIK |
| | | | LVTKYPMDNVINFINQDVRYSPTFIKTIKDFVNEHLPTMYDGLNDYLHSV |
| | | | IIDEDLIEEYKIKSVAMFNLEYKTDIDTLTLDEQIFVEVNISYYDFRYRQFA |
| | | | DEFRDYIMIKERRQITMQSGDRIRRFRRPMSLRSTIIKKDTDSXEDILSXID |
| | | | NAKKNSKVSIEDVERIIXSFRLNPXVVRXTMLXDIDIKTKXMVLKIAKDW |
| | | | KSCTLTLSVKGIXVTDTINTVLSKILHHHRNIFKYLTSVENKEIXVCNCSR |
| | | | CLSLFYRKLKSVRCDLRTDDGLLDRLYDLTRYALHGKINQNLIGQRCWG |
| | | | PLTEILFNENKKKKLNNLMEYIKISDMLVYGHYIEKTLIPITDSLSFKLFVD |
| | | | TMSVLNDQYAKVVIFFNTIIEYIIATIYYRLTVLDNYTNVKHFVSKVLHTV |
| | | | MEACGVLFSHIKVNDKIEYELEEMVDKGTVPSYLYHLSINVISIILDDING |
| | | | TR |
| 140 | Q1L | NC_003310.1 | MFMYPEFARKALSKLISKKLNIEKVSSKHQLVLLDYGLHGLLPKSLYLEA |
| | | | INSDILNVRFFPPEIINVTDIVKALQNSCRVDEYLKAVSLYHKNSLMVSGP |
| | | | NVVKLMIEYNLLTHSDLEWLINENVVKATYLLKINAYMINFKIDLTVDEII |
| | | | DLVKDIPVGATLHLYNILNNIDLDIILRISDEYNIPPVHDILSKLTDEEMCIK |
| | | | LVTKYPMDNVINFINQDVRYSPTFIKTIKDFVNEHLPTMYDGLNDYLHSV |
| | | | IIDEDLIEEYKIKSVAMFNLEYKTDIDTLTLDEQIFVEVNISYYDFRYRQFA |
| | | | DEFRDYIMIKERRQITMQSGDRIRRFRRPMSLRSTIIKKDTDSLEDILSHID |
| | | | NAKKNSKVSIEDVERIISSFRLNPCVVRRTMLSDIDIKTKIMVLKIAKDWK |
| | | | SCTLTLSVKGIMVTDTINTVLSKILHHHRNVFKYLTSVENKEIAVCNCSRC |
| | | | LSLFYRKLKSVRCDLRTDDGLLDRLYDLTRYALHGKINQNLIGQRCWGP |
| | | | LTEILFNENKKKKLNNLMEYIKISDMLVYGHYIEKTLIPITDSLSFKLFVDT |
| | | | MSVLNDQYAKVVIFFNTIIEYIIATIYYRLTVLDNYTNVKHFVSKVLHTV |
| | | | MEACGVLFSHIKVNDKIEYELEEMVDKGTVPSYLYHLSINVISIILDDING |
| | | | TR |
| 141 | Q1L | Belgian | MFMYPEFARKALSKLISKKLNIEKVSSKHQLVLLDYGLHGLLPKSLYLEA |
| | | Outbreak | INSDILNVRFFPPEIINVTDIVKALQNSCRVDEYLKAVSLYHKNSLMVSGP |
| | | (Belgium20 | NVVKLMIEYNLLTHSDLEWLINENVVKATYLLKINAYMINFKIDLTVDEII |
| | | 220513) | DLVKDIPVGATLHLYNILNNIDLDIILRISDEYNIPPVHDILSKLTNEEMCIK |
| | | | LVTKYPMDNVINFINQDVRYSPTFIKTIKDFVNEHLPTMYDGLNDYLHSV |
| | | | IIDEDLIEEYKIKSVAMFNLEYKTDIDTLTLDEQIFVEVNISYYDFRYRQFA |
| | | | DEFRDYIMIKERRQITMQSGDRIRRFRRPMSLRSTIIKKDTDSXEDILSXID |
| | | | NAKKNSKVSIEDVERIIXSFRLNPXVVRXTMLXDIDIKTKXMVLKIAKDW |
| | | | KSCTLTLSVKGIXVTDTINTVLSKILHHHRNIFKYLTSVENKEIXVCNCSR |
| | | | CLSLFYRKLKSVRCDLRTDDGLLDRLYDLTRYALHGKINQNLIGQRCWG |
| | | | PLTEILFNENKKKKLNNLMEYIKISDMLVYGHYIEKTLIPITDSLSFKLFVD |
| | | | TMSVLNDQYAKVVIFFNTIIEYIIATIYYRLTVLDNYTNVKHFVSKVLHTV |
| | | | MEACGVLFSHIKVNDKIEYELEEMVDKGTVPSYLYHLSINVISIILDDING |
| | | | TR |
| 244 | B12R | Outbreak | MDIFREIASSMKGKNVFISPASISSVLTILYYGANGSTAEQLSKYVEKEEN |
| | | Consensus | MDKVSAQNISFKSMNKVYGRYSAVFKDSFLGKIGDKFQTVDFTDCRTID |
| | | | AINKCVDIFTEGKINPLLDEPLSPDTCLLAISAVYFKAKWLMPFEKEFTSD |
| | | | YPFYVSPTEMVDVSMMSIYGEPFNHASVKESFGNFSIIELPYVGDTSMMV |
| | | | ILPNKIDGLESIEQNLTDTNFKKWCNSLKATFIDVHIPKFKVIGSYNLVDT |
| | | | LIKLGLTDVFYSTGDYINMCNSDVSVDAMIHKTYIDVNEEYTEAAAATS |
| | | | VLVADCASTVTNEFCADHPFIYVIRHVDGKILFVGRYCSPTTN |
| 245 | B12R | Portuguese | MDIFREIASSMKGKNVFISPASISSVLTILYYGANGSTAEQLSKYVEKEEN |
| | | Outbreak | MDKVSAQNISFKSMNKVYGRYSAVFKDSFLGKIGDKFQTVDFTDCRTID |
| | | (Portugal20 | AINKCVDIFTEGKINPLLDEPLSPDTCLLAISAVYFKAKWLMPFEKEFTSD |
| | | 220504) | YPFYVSPTEMVDVSMMSIYGEPFNHASVKESFGNFSIIELPYVGDTSMMV |
| | | | ILPNKIDGLESIEQNLTDTNFKKWCNSLKATFIDVHIPKFKVIGSYNLVDT |
| | | | LIKLGLTDVFYSTGDYINMCNSDVSVDAMIHKTYIDVNEEYTEAAAATS |
| | | | VLVADCASTVTNEFCADHPFIYVIRHVDGKILFVGRYCSPTTN |
| 246 | B12R | Belgian | MDIFREIASSMKGKNVFISPASISSVLTILYYGANGSTAEQLSKYVEKEEN |
| | | Outbreak | MDKVSAQNISFKSMNKVYGRYSAVFKDSFLGKIGDKFQTVDFTDCRTID |
| | | (Belgium20 | AINKCVDIFTEGKINPLLDEPLSPDTCLLAISAVYFKAKWLMPFEKEFTSD |
| | | 220513) | YPFYVSPTEMVDVSMMSIYGEPFNHASVKESFGNFSIIELPYVGDTSMMV |
| | | | ILPNKIDGLESIEQNLTDTNFKKWCNSLKATFIDVHIPKFKVIGSYNLVDT |
| | | | LIKLGLTDVFYSTGDYINMCNSDVSVDAMIHKTYIDVNEEYTEAAAATS |
| | | | VLVADCASTVTNEFCADHPFIYVIRHVDGKILFVGRYCSPTTN |
| 247 | B12R | NC_003310.1 | MDIFREIASSMKGKNVFISPASISSVLTILYYGANGSTAEQLSKYVEKEEN |
| | | | MDKVSAQNISFKSMNKVYGRYSAVFKDSFLGKIGDKFQTVDFTDCRTID |
| | | | AINKCVDIFTEGKINPLLDEPLSPDTCLLAISAVYFKAKWLMPFEKEFTSD |
| | | | YPFYVSPTEMVDVSMMSIYGEPFNHASVKESFGNFSIIELPYVGDTSMMV |
| | | | ILPNKIDGLESIEQNLTDTNFKKWCNSLKATFIDVHIPKFKVIGSYNLVDT |
| | | | LIKLGLTDVFYSTGDYINMCNSDVSVDAMIHKTYIDVNEEYTEAAAATS |
| | | | VLVADCASTVTNEFCADHPFIYVIRHVDGKILFVGRYCSPTTN |
| 248 | C17L | Outbreak | MGFCIPLRSKMLKRVSRKSSSILARRPTPKKMNIVTDSENRLKKNSYIENT |
| | | Consensus | NOGNILMDSIFVSTMPVETLFGSYITDDNDDYELKDLLNVTYNIKPVIVPD |
| | | | IKLDSVLDRDGNFRPADCFLVKLKHSDGFTKGALYLGHSAGFTATICLKN |
| | | | EGVSGLYIPGTSVVRSNICQGDTIVSRSSRGVQFLPQIGGEAIFLIVSLCPTK |
| | | | KLVETGFVIPEISSNDNAKIAARILSEKRKDIIAHINTLIQYRQQLELAYYN |
| | | | SCMLTEFLHYCNSYADTIKESLLKETIQKDINIIHTNITTLLNETAKVIKLV |
| | | | KSLVDKEDTDIVNNFITKEIKNCGGVKNRDKIVNSLSLSNLDFRL |
| 249 | C17L | Portuguese | MGFCIPLRSKMLKRVSRKSSSILARRPTPKKMNIVTDSENRLKKNSYIENT |
| | | Outbreak | NQGNILMDSIFVSTMPVETLFGSYITDDNDDYELKDLLNVTYNIKPVIVPD |
| | | (Portugal20 | IKLDSVLDRDGNFRPADCFLVKLKHSDGFTKGALYLGHSAGFTATICLKN |
| | | 220504) | EGVSGLYIPGTSVVRSNICQGDTIVSRSSRGVQFLPQIGGEAIFLIVSLCPTK |
| | | | KLVETGFVIPEISSNDNAKIAARILSEKRKDIIAHINTLIQYRQQLELAYYN |
| | | | SCMLTEFLHYCNSYADTIKESLLKETIQKDINIIHTNITTLLNETAKVIKLV |
| | | | KSLVDKEDTDIVNNFITKEIKNCGGVKNRDKIVNSLSLSNLDFRL |
| 250 | C17L | Belgian | MGFCIPLRSKMLKRVSRKSSSILARRPTPKKMNIVTDSENRLKKNSYIENT |
| | | Outbreak | NOGNILMDSIFVSTMPVETLFGSYITDDNDDYELKDLLNVTYNIKPVIVPD |
| | | (Belgium20 | IKLDSVLDRDGNFRPADCFLVKLKHSDGFTKGALYLGHSAGFTATICLKN |
| | | 220513) | EGVSGLYIPGTSVVRSNICQGDTIVSRSSRGVQFLPQIGGEAIFLIVSLCPTK |
| | | | KLVETGFVIPEISSNDNAKIAARILSEKRKDIIAHINTLIQYRQQLELAYYN |
| | | | SCMLTEFLHYCNSYADTIKESLLKETIQKDINIIHTNITTLLNETAKVIKLV |
| | | | KSLVDKEDTDIVNNFITKEIKNCGGVKNRDKIVNSLSLSNLDFRL |
| 251 | C17L | NC_003310.1 | MGFCIPLRSKMLKRVSRKSSSILARRPTPKKMNIVTDSENRLKKNSYIENT |
| | | | NQGNILMDSIFVSTMPVETLFGSYITDDNDDYELKDLLNVTYNIKPVIVPD |
| | | | IKLDSVLDRDGNFRPADCFLVKLKHSDGFTKGALYLGHSAGFTATICLKN |
| | | | EGVSGLYIPGTSVVRSNICQGDTIVSRSSRGVQFLPQIGGEAIFLIVSLCPTK |
| | | | KLVETGFVIPEISSNDNAKIAARILSEKRKDIIAHINTLIQYRQQLELAYYN |
| | | | SCMLTEFLHYCNSYADTIKESLLKETIQKDINIIHTNITTLLNETAKVIKLV |
| | | | KSLVDKEDTDIVNNFITKEIKNCGGVKNRDKIVNSLSLSNLDFRL |

TABLE 9
Table of Exemplary Construct Sequences

| SEQ | | AA/ | |
|---|---|---|---|
| ID NO | Description | NT | Sequence |
| 158 | M1R antigen | AA | MGAAASIQTTVNTLSERISSKLEQEANASAQTKCDIEIGNFYIRQNHGC |
| | | | NITVKNMCSADADAQLDAVLSAATETYSGLTPEQKAYVPAMFTAAL |
| | | | NIQTSVNTVVRDFENYVKQTCNSSAVVDNKLKIQNVIIDECYGAPGSP |
| | | | TNLEFINTGSSKGNCAIKALMQLTTKATTQIAPRQVAGTGVQFYMIVI |
| | | | GVIILAALFMYYAKRMLFTSTNDKIKLILANKENVHWTTYMDTFFRT |
| | | | SPMIIATTDIQN |
| 159 | M1R antigen | NT | ATGGGTGCCGCAGCAAGCATACAGACGACTGTGAATACACTCAGT |
| | | | GAACGTATCTCGTCTAAATTAGAACAAGAAGCGAACGCTAGTGCT |
| | | | CAAACAAAATGTGATATAGAAATCGGAAATTTTTATATCCGACAA |
| | | | AACCATGGATGTAACATCACTGTTAAAAATATGTGCTCTGCGGAC |
| | | | GCGGATGCTCAGTTGGATGCTGTGTTATCAGCCGCTACAGAAACA |
| | | | TATAGTGGATTAACACCGGAACAAAAAGCATACGTACCAGCTATG |
| | | | TTTACTGCTGCGTTAAACATTCAGACGAGTGTAAACACTGTTGTTA |
| | | | GAGATTTTGAAAATTATGTGAAACAGACTTGTAATTCTAGCGCTGT |
| | | | TGTCGATAACAAATTAAAGATACAAAACGTAATTATAGATGAATG |
| | | | TTACGGAGCCCCAGGATCTCCAACAAATTTGGAATTTATTAATACA |
| | | | GGATCTAGCAAAGGAAATTGTGCCATTAAGGCGTTGATGCAATTG |
| | | | ACTACTAAGGCCACTACTCAAATAGCACCTAGACAAGTTGCTGGT |
| | | | ACAGGAGTTCAGTTTTATATGATTGTTATCGGTGTTATAATATTGG |
| | | | CAGCGTTGTTTATGTACTATGCCAAGCGTATGCTGTTCACATCCAC |
| | | | CAATGATAAAATCAAACTTATTTTAGCCAATAAGGAAAACGTCCA |
| | | | TTGGACTACTTACATGGACACATTCTTTAGAACTTCTCCGATGATT |
| | | | ATTGCTACCACGGATATACAAAACTGA |
| 160 | HSV/gDsec (SS) | AA | MGGAAARLGAVILFVVIVGLHGVRG |
| 161 | HSV/gDsec (SS) | NT | atggggggggctgccgccaggttgggggccgtgattttgtttgtcgtcatagtgggcctccatggggtccgcg |
| | | | gc |
| 162 | SS + MIR | AA | MGGAAARLGAVILFVVIVGLHGVRGMGAAASIQTTVNTLSERISSKL |
| | | | EQEANASAQTKCDIEIGNFYIRQNHGCNITVKNMCSADADAQLDAVL |
| | | | SAATETYSGLTPEQKAYVPAMFTAALNIQTSVNTVVRDFENYVKQTC |
| | | | NSSAVVDNKLKIQNVIIDECYGAPGSPTNLEFINTGSSKGNCAIKALM |
| | | | QLTTKATTQIAPRQVAGTGVQFYMIVIGVIILAALFMYYAKRMLFTST |
| | | | NDKIKLILANKENVHWTTYMDTFFRTSPMIIATTDIQN |
| 163 | SS + MIR | NT | atggggggggctgccgccaggttgggggccgtgattttgtttgtcgtcatagtgggcctccatggggtccgcg |
| | | | gcATGGGTGCCGCAGCAAGCATACAGACGACTGTGAATACACTCA |
| | | | GTGAACGTATCTCGTCTAAATTAGAACAAGAAGCGAACGCTAGTG |
| | | | CTCAAACAAAATGTGATATAGAAATCGGAAATTTTTATATCCGAC |
| | | | AAAACCATGGATGTAACATCACTGTTAAAAATATGTGCTCTGCGG |
| | | | ACGCGGATGCTCAGTTGGATGCTGTGTTATCAGCCGCTACAGAAA |
| | | | CATATAGTGGATTAACACCGGAACAAAAAGCATACGTACCAGCTA |
| | | | TGTTTACTGCTGCGTTAAACATTCAGACGAGTGTAAACACTGTTGT |
| | | | TAGAGATTTTGAAAATTATGTGAAACAGACTTGTAATTCTAGCGCT |
| | | | GTTGTCGATAACAAATTAAAGATACAAAACGTAATTATAGATGAA |
| | | | TGTTACGGAGCCCCAGGATCTCCAACAAATTTGGAATTTATTAATA |
| | | | CAGGATCTAGCAAAGGAAATTGTGCCATTAAGGCGTTGATGCAAT |
| | | | TGACTACTAAGGCCACTACTCAAATAGCACCTAGACAAGTTGCTG |
| | | | GTACAGGAGTTCAGTTTTATATGATTGTTATCGGTGTTATAATATT |
| | | | GGCAGCGTTGTTTATGTACTATGCCAAGCGTATGCTGTTCACATCC |
| | | | ACCAATGATAAAATCAAACTTATTTTAGCCAATAAGGAAAACGTC |
| | | | CATTGGACTACTTACATGGACACATTCTTTAGAACTTCTCCGATGA |
| | | | TTATTGCTACCACGGATATACAAAACTGA |
| 164 | A29L antigen | AA | MDGTLFPGDDDLAIPATEFFSTKAAKNPETKREAIVKAYGDDNEETL |
| | | | KQRLTNLEKKITNITTKFEQIEKCCKRNDEVLFRLENHAETLRAAMISL |
| | | | AKKIDVQTGRHPYE |
| 165 | A29L antigen | NT | ATGGACGGAACTCTTTTCCCCGGAGATGACGATCTTGCAATTCCAG |
| | | | CAACTGAATTTTTCTCTACAAAGGCTGCTAAAAATCCAGAGACTA |
| | | | AACGCGAAGCAATTGTTAAAGCCTATGGAGACGATAATGAGGAAA |
| | | | CTCTCAAACAACGGCTAACTAATTTGGAAAAAAAGATTACTAATA |
| | | | TAACAACAAAGTTTGAACAAATAGAAAAGTGTTGTAAACGCAACG |
| | | | ATGAAGTTCTATTTAGGTTGGAAAATCACGCTGAAACTCTAAGAG |
| | | | CGGCTATGATATCTCTGGCTAAAAAGATTGATGTACAGACTGGAC |
| | | | GGCATCCATATGAGTAA |
| 166 | SS + A29L | AA | MGGAAARLGAVILFVVIVGLHGVRGMDGTLFPGDDDLAIPATEFFST |
| | | | KAAKNPETKREAIVKAYGDDNEETLKQRLTNLEKKITNITTKFEQIEK |
| | | | CCKRNDEVLFRLENHAETLRAAMISLAKKIDVQTGRHPYE |
| 167 | SS + A29L | NT | atggggggggctgccgccaggttgggggccgtgattttgtttgtcgtcatagtgggcctccatggggtccgcg |
| | | | gcATGGACGGAACTCTTTTCCCCGGAGATGACGATCTTGCAATTCC |
| | | | AGCAACTGAATTTTTCTCTACAAAGGCTGCTAAAAATCCAGAGAC |
| | | | TAAACGCGAAGCAATTGTTAAAGCCTATGGAGACGATAATGAGGA |
| | | | AACTCTCAAACAACGGCTAACTAATTTGGAAAAAAAGATTACTAA |
| | | | TATAACAACAAAGTTTGAACAAATAGAAAAGTGTTGTAAACGCAA |
| | | | CGATGAAGTTCTATTTAGGTTGGAAAATCACGCTGAAACTCTAAG |
| | | | AGCGGCTATGATATCTCTGGCTAAAAAGATTGATGTACAGACTGG |
| | | | ACGGCATCCATATGAGTAA |
| 168 | A29L(C71A C72A) | AA | MDGTLFPGDDDLAIPATEFFSTKAAKNPETKREAIVKAYGDDNEETL |
| | | | KQRLTNLEKKITNITTKFEQIEKAAKRNDEVLFRLENHAETLRAAMIS |
| | | | LAKKIDVQTGRHPYE |
| 169 | A29L(C71A C72A) | NT | ATGGACGGAACTCTTTTCCCCGGAGATGACGATCTTGCAATTCCAG |
| | | | CAACTGAATTTTTCTCTACAAAGGCTGCTAAAAATCCAGAGACTA |
| | | | AACGCGAAGCAATTGTTAAAGCCTATGGAGACGATAATGAGGAAA |
| | | | CTCTCAAACAACGGCTAACTAATTTGGAAAAAAAGATTACTAATA |
| | | | TAACAACAAAGTTTGAACAAATAGAAAAGGCTGCTAAACGCAACG |
| | | | ATGAAGTTCTATTTAGGTTGGAAAATCACGCTGAAACTCTAAGAG |
| | | | CGGCTATGATATCTCTGGCTAAAAAGATTGATGTACAGACTGGAC |
| | | | GGCATCCATATGAGTAA |
| 170 | SS + | AA | MGGAAARLGAVILFVVIVGLHGVRGMDGTLFPGDDDLAIPATEFFST |
| | A29L(C71A C72A) | | KAAKNPETKREAIVKAYGDDNEETLKQRLTNLEKKITNITTKFEQIEK |
| | | | AAKRNDEVLFRLENHAETLRAAMISLAKKIDVQTGRHPYE |
| 171 | SS + | NT | atggggggggctgccgccaggttgggggccgtgattttgtttgtcgtcatagtgggcctccatggggtccgcg |
| | A29L(C71A C72A) | | gcATGGACGGAACTCTTTTCCCCGGAGATGACGATCTTGCAATTCC |
| | | | AGCAACTGAATTTTTCTCTACAAAGGCTGCTAAAAATCCAGAGAC |
| | | | TAAACGCGAAGCAATTGTTAAAGCCTATGGAGACGATAATGAGGA |
| | | | AACTCTCAAACAACGGCTAACTAATTTGGAAAAAAAGATTACTAA |
| | | | TATAACAACAAAGTTTGAACAAATAGAAAAGGCTGCTAAACGCAA |
| | | | CGATGAAGTTCTATTTAGGTTGGAAAATCACGCTGAAACTCTAAG |
| | | | AGCGGCTATGATATCTCTGGCTAAAAAGATTGATGTACAGACTGG |
| | | | ACGGCATCCATATGAGTAA |
| 172 | A35R | AA | MMTPENDEEQTSVFSATVYGDKIQGKNKRKRVIGLCIRISMVISLLSM |
| | | | ITMSAFLIVRLNQCMSANKAAITDSAVAVAAASSTHRKVVSSTTQYD |
| | | | HKESCNGLYYQGSCYILHSDYKSFEDAKANCAAESSTLPNKSDVLTT |
| | | | WLIDYVEDTWGSDGNPITKTTSDYQDSDVSQEVRKYFCT |
| 173 | A35R | NT | ATGATGACACCAGAAAACGACGAAGAGCAGACATCTGTGTTCTCC |
| | | | GCTACTGTTTACGGAGACAAAATTCAGGGAAAGAATAAACGCAAA |
| | | | CGCGTGATTGGTCTATGTATTAGAATATCTATGGTTATTTCACTAC |
| | | | TATCTATGATTACCATGTCCGCGTTTCTCATAGTGCGCCTAAATCA |
| | | | ATGCATGTCTGCTAACAAGGCTGCTATTACTGACTCCGCTGTTGCC |
| | | | GTTGCTGCGGCATCATCTACTCATAGAAAGGTTGTGTCTAGCACTA |
| | | | CACAATATGATCACAAAGAAAGCTGTAATGGTTTATATTACCAGG |
| | | | GTTCTTGTTATATATTACATTCAGACTATAAGTCATTCGAGGATGC |
| | | | TAAAGCAAACTGCGCTGCGGAATCATCAACACTACCCAATAAATC |
| | | | CGATGTCTTGACTACCTGGCTCATTGATTATGTTGAGGATACATGG |
| | | | GGATCTGATGGTAATCCAATTACAAAAACTACATCCGATTATCAA |
| | | | GATTCTGATGTATCACAAGAAGTTAGAAAGTATTTTTGTACATAA |
| 174 | A35R ECD(89-181) | AA | SSTTQYDHKESCNGLYYQGSCYILHSDYKSFEDAKANCAAESSTLPN |
| | | | KSDVLTTWLIDYVEDTWGSDGNPITKTTSDYQDSDVSQEVRKYFCT |
| 175 | A35R ECD(89-181) | NT | TCTAGCACTACACAATATGATCACAAAGAAAGCTGTAATGGTTTA |
| | | | TATTACCAGGGTTCTTGTTATATATTACATTCAGACTATAAGTCAT |
| | | | TCGAGGATGCTAAAGCAAACTGCGCTGCGGAATCATCAACACTAC |
| | | | CCAATAAATCCGATGTCTTGACTACCTGGCTCATTGATTATGTTGA |
| | | | GGATACATGGGGATCTGATGGTAATCCAATTACAAAAACTACATC |
| | | | CGATTATCAAGATTCTGATGTATCACAAGAAGTTAGAAAGTATTTT |
| | | | TGTACA |
| 176 | Linker | AA | GGSGGGGSGG |
| 177 | Linker | NT | ggcggctctggaggaggcggctccggaggc |
| 178 | SS + A35R ECD + | AA | MGGAAARLGAVILFVVIVGLHGVRGSSTTQYDHKESCNGLYYQGSC |
| | Linker + A35R ECD | | YILHSDYKSFEDAKANCAAESSTLPNKSDVLTTWLIDYVEDTWGSDG |
| | | | NPITKTTSDYQDSDVSQEVRKYFCTGGSGGGGSGGSSTTQYDHKESC |
| | | | NGLYYQGSCYILHSDYKSFEDAKANCAAESSTLPNKSDVLTTWLIDY |
| | | | VEDTWGSDGNPITKTTSDYQDSDVSQEVRKYFCT |
| 179 | SS + A35R ECD + | NT | atggggggggctgccgccaggttgggggccgtgattttgtttgtcgtcatagtgggcctccatggggtccgcg |
| | Linker + A35R ECD | | gcTCTAGCACTACACAATATGATCACAAAGAAAGCTGTAATGGTTT |
| | | | ATATTACCAGGGTTCTTGTTATATATTACATTCAGACTATAAGTCA |
| | | | TTCGAGGATGCTAAAGCAAACTGCGCTGCGGAATCATCAACACTA |
| | | | CCCAATAAATCCGATGTCTTGACTACCTGGCTCATTGATTATGTTG |
| | | | AGGATACATGGGGATCTGATGGTAATCCAATTACAAAAACTACAT |
| | | | CCGATTATCAAGATTCTGATGTATCACAAGAAGTTAGAAAGTATTT |
| | | | TTGTACAggcggctctggaggaggcggctccggaggcTCTAGCACTACACAATATG |
| | | | ATCACAAAGAAAGCTGTAATGGTTTATATTACCAGGGTTCTTGTTA |
| | | | TATATTACATTCAGACTATAAGTCATTCGAGGATGCTAAAGCAAA |
| | | | CTGCGCTGCGGAATCATCAACACTACCCAATAAATCCGATGTCTTG |
| | | | ACTACCTGGCTCATTGATTATGTTGAGGATACATGGGGATCTGATG |
| | | | GTAATCCAATTACAAAAACTACATCCGATTATCAAGATTCTGATGT |
| | | | ATCACAAGAAGTTAGAAAGTATTTTTGTACATAA |
| 180 | B6 | AA | MKTISVVTLLCVLPAVVYSTCTVPTMNNAKLTSTETSFNDKQKVTFT |
| | | | CDSGYHSLDPNAVCETDKWKYENPCKKMCTVSDYVSELYDKPLYEV |
| | | | NSTMTLSCNGETKYFRCEEKNGNTSWNDTVTCPNAECQPLQLEHGSC |
| | | | QPVKEKYSFGEYMTINCDVGYEVIGVSYISCTANSWNVIPSCQQKCDI |
| | | | PSLSNGLISGSTFSIGGVIHLSCKSGFTLTGSPSSTCIDGKWNPILPTCVR |
| | | | SNEEFDPVDDGPDDETDLSKLSKDVVQYEQEIESLEATYHIIIMALTIM |
| | | | GVIFLISIIVLVCSCDKNNDQYKFHKLLP |
| 181 | B6 | NT | atgaaaacgatttccgttgttacgttgttatgcgtactacctgctgttgtttattcaacatgtactgtacccactatgaa |
| | | | taacgctaaattaacgtctaccgaaacatcgtttaatgataaacagaaagttacgtttacatgtgattcaggatatc |
| | | | attctttggatccaaatgctgtctgtgaaacagataaatggaaatacgaaaatccatgcaagaaaatgtgcacag |
| | | | tttctgattatgtctctgaactatatgataagccattatacgaagtgaattccaccatgacactaagttgcaacggtg |
| | | | aaacaaaatattttcgttgtgaagaaaaaaatggaaatacttcttggaatgatactgtcacgtgtcctaatgcgga |
| | | | atgtcaacctcttcaattagaacacggatcgtgtcaaccagttaaagaaaaatactcatttggggaatatatgact |
| | | | atcaactgtgatgttggatatgaggttattggtgtttcgtatataagttgtacggctaattcttggaatgttattccatc |
| | | | atgtcaacaaaaatgtgatataccgtccctatctaatggattaatttccggatctacattttctatcggtggcgttata |
| | | | catcttagttgtaaaagtggttttacactaacggggtctccatcatccacatgtatcgacggtaaatggaatcccat |
| | | | actcccaacatgtgtacgatctaacgaagaatttgatccagtggatgatggtcccgacgatgagacagatctga |
| | | | gcaaactctcgaaagacgttgtacaatatgaacaagaaatagaatcgttagaagcaacttatcatataatcataat |
| | | | ggcgttgacaattatgggtgtcatatttctaatctccattatagtattagtttgttcctgtgacaaaaataatgaccaat |
| | | | ataagttccataaattgctaccgtga |
| 182 | B6 (C140A) | AA | MKTISVVTLLCVLPAVVYSTCTVPTMNNAKLTSTETSFNDKQKVTFT |
| | | | CDSGYHSLDPNAVCETDKWKYENPCKKMCTVSDYVSELYDKPLYEV |
| | | | NSTMTLSCNGETKYFRCEEKNGNTSWNDTVTCPNAECQPLQLEHGS |
| | | | AQPVKEKYSFGEYMTINCDVGYEVIGVSYISCTANSWNVIPSCQQKC |
| | | | DIPSLSNGLISGSTFSIGGVIHLSCKSGFTLTGSPSSTCIDGKWNPILPTC |
| | | | VRSNEEFDPVDDGPDDETDLSKLSKDVVQYEQEIESLEATYHIIIMALT |
| | | | IMGVIFLISIIVLVCSCDKNNDQYKFHKLLP |
| 183 | B6 (C140A) | NT | atgaaaacgatttccgttgttacgttgttatgcgtactacctgctgttgtttattcaacatgtactgtacccactatgaa |
| | | | taacgctaaattaacgtctaccgaaacatcgtttaatgataaacagaaagttacgtttacatgtgattcaggatatc |
| | | | attctttggatccaaatgctgtctgtgaaacagataaatggaaatacgaaaatccatgcaagaaaatgtgcacag |
| | | | tttctgattatgtctctgaactatatgataagccattatacgaagtgaattccaccatgacactaagttgcaacggtg |
| | | | aaacaaaatattttcgttgtgaagaaaaaaatggaaatacttcttggaatgatactgtcacgtgtcctaatgcgga |
| | | | atgtcaacctcttcaattagaacacggatcgGCTcaaccagttaaagaaaaatactcatttggggaatatatga |
| | | | ctatcaactgtgatgttggatatgaggttattggtgtttcgtatataagttgtacggctaattcttggaatgttattcca |
| | | | tcatgtcaacaaaaatgtgatataccgtccctatctaatggattaatttccggatctacattttctatcggtggcgtta |
| | | | tacatcttagttgtaaaagtggttttacactaacggggtctccatcatccacatgtatcgacggtaaatggaatccc |
| | | | atactcccaacatgtgtacgatctaacgaagaatttgatccagtggatgatggtcccgacgatgagacagatctg |
| | | | agcaaactctcgaaagacgttgtacaatatgaacaagaaatagaatcgttagaagcaacttatcatataatcata |
| | | | atggcgttgacaattatgggtgtcatatttctaatctccattatagtattagtttgttcctgtgacaaaaataatgacca |
| | | | atataagttccataaattgctaccgtga |
| 184 | H3L | AA | MAAVKTPVIVVPVIDRPPSETFPNVHEHINDQKFDDVKDNEVMQEKR |
| | | | DVVIVNDDPDHYKDYVFIQWTGGNIRDDDKYTHFFSGFCNTMCTEET |
| | | | KRNIARHLALWDSKFFTELENKNVEYVVIIENDNVIEDITFLRPVLKAI |
| | | | HDKKIDILQMREIITGNKVKTELVIDKDHAIFTYTGGYDVSLSAYIIRV |
| | | | TTALNIVDEIIKSGGLSSGFYFEIARIENEMKINRQIMDNSAKYVEHDP |
| | | | RLVAEHRFETMKPNFWSRIGTVAAKRYPGVMYTFTTPLISFFGLFDIN |
| | | | VIGLIVILFIMFMLIFNVKSKLLWFLTGTFVTAFI |
| 185 | H3L | NT | atggcggcggtgaaaactcctgttattgttgtgccagttattgatagacctccatcagaaacatttcctaatgttcat |
| | | | gagcatattaatgatcagaagttcgatgatgtgaaggacaatgaagttatgcaagaaaaaagagatgttgtgata |
| | | | gtcaatgatgatccagatcattacaaggattatgtgtttatacaatggactggaggaaatattagagatgatgaca |
| | | | agtatactcacttcttttcagggttttgtaacactatgtgtacagaggaaacgaaaagaaatatcgctagacattta |
| | | | gccctatgggattctaaattttttaccgagctagaaaataaaaatgtagaatatgtagttattatagaaaacgataa |
| | | | cgttattgaggatattacgtttcttcgtcccgtcttgaaggcaatacatgacaagaaaatagatatcctacagatga |
| | | | gagaaattattacaggaaataaagttaaaaccgagcttgtaatagacaaagatcatgccatatttacatatacagg |
| | | | agggtatgatgttagcttatccgcctatattattagagttactacggcgctgaacatcgtagatgaaattataaagt |
| | | | ctggaggtctatcatcgggattttattttgaaatagccagaatcgaaaacgaaatgaagattaacaggcaaataat |
| | | | ggataactctgccaaatatgtagaacacgatcctcgtcttgttgcagaacaccgcttcgaaaccatgaaaccaa |
| | | | atttttggtctagaataggaacggtagctgctaaacgttatccaggagttatgtacacgtttactactccattgatttc |
| | | | attttttggattgtttgatattaatgttataggtttgattgtaattttgtttattatgtttatgctcatctttaatgttaaatcta |
| | | | aactgttatggttccttacaggaacatttgttaccgcatttatctaa |
| 186 | SS + H3L | AA | MGGAAARLGAVILFVVIVGLHGVRGMAAVKTPVIVVPVIDRPPSETFP |
| | | | NVHEHINDQKFDDVKDNEVMQEKRDVVIVNDDPDHYKDYVFIQWT |
| | | | GGNIRDDDKYTHFFSGFCNTMCTEETKRNIARHLALWDSKFFTELEN |
| | | | KNVEYVVIIENDNVIEDITFLRPVLKAIHDKKIDILQMREIITGNKVKTE |
| | | | LVIDKDHAIFTYTGGYDVSLSAYIIRVTTALNIVDEIIKSGGLSSGFYFEI |
| | | | ARIENEMKINRQIMDNSAKYVEHDPRLVAEHRFETMKPNFWSRIGTV |
| | | | AAKRYPGVMYTFTTPLISFFGLFDINVIGLIVILFIMFMLIFNVKSKLLW |
| | | | FLTGTFVTAFI |
| 187 | SS + H3L | NT | atggggggggctgccgccaggttgggggccgtgattttgtttgtcgtcatagtgggcctccatggggtccgcg |
| | | | gcatggcggcggtgaaaactcctgttattgttgtgccagttattgatagacctccatcagaaacatttcctaatgtt |
| | | | catgagcatattaatgatcagaagttcgatgatgtgaaggacaatgaagttatgcaagaaaaaagagatgttgtg |
| | | | atagtcaatgatgatccagatcattacaaggattatgtgtttatacaatggactggaggaaatattagagatgatg |
| | | | acaagtatactcacttcttttcagggttttgtaacactatgtgtacagaggaaacgaaaagaaatatcgctagacat |
| | | | ttagccctatgggattctaaattttttaccgagctagaaaataaaaatgtagaatatgtagttattatagaaaacgat |
| | | | aacgttattgaggatattacgtttcttcgtcccgtcttgaaggcaatacatgacaagaaaatagatatcctacagat |
| | | | gagagaaattattacaggaaataaagttaaaaccgagcttgtaatagacaaagatcatgccatatttacatataca |
| | | | ggagggtatgatgttagcttatccgcctatattattagagttactacggcgctgaacatcgtagatgaaattataaa |
| | | | gtctggaggtctatcatcgggattttattttgaaatagccagaatcgaaaacgaaatgaagattaacaggcaaat |
| | | | aatggataactctgccaaatatgtagaacacgatcctcgtcttgttgcagaacaccgcttcgaaaccatgaaacc |
| | | | aaatttttggtctagaataggaacggtagctgctaaacgttatccaggagttatgtacacgtttactactccattgat |
| | | | ttcattttttggattgtttgatattaatgttataggtttgattgtaattttgtttattatgtttatgctcatctttaatgttaaatc |
| | | | taaactgttatggttccttacaggaacatttgttaccgcatttatctaa |
| 188 | H3L (C86A C90A) | AA | MAAVKTPVIVVPVIDRPPSETFPNVHEHINDQKFDDVKDNEVMQEKR |
| | | | DVVIVNDDPDHYKDYVFIQWTGGNIRDDDKYTHFFSGFANTMATEE |
| | | | TKRNIARHLALWDSKFFTELENKNVEYVVIIENDNVIEDITFLRPVLKA |
| | | | IHDKKIDILQMREIITGNKVKTELVIDKDHAIFTYTGGYDVSLSAYIIRV |
| | | | TTALNIVDEIIKSGGLSSGFYFEIARIENEMKINRQIMDNSAKYVEHDP |
| | | | RLVAEHRFETMKPNFWSRIGTVAAKRYPGVMYTFTTPLISFFGLFDIN |
| | | | VIGLIVILFIMFMLIFNVKSKLLWFLTGTFVTAFI |
| 189 | H3L (C86A C90A) | NT | atggcggcggtgaaaactcctgttattgttgtgccagttattgatagacctccatcagaaacatttcctaatgttcat |
| | | | gagcatattaatgatcagaagttcgatgatgtgaaggacaatgaagttatgcaagaaaaaagagatgttgtgata |
| | | | gtcaatgatgatccagatcattacaaggattatgtgtttatacaatggactggaggaaatattagagatgatgaca |
| | | | agtatactcacttcttttcagggtttGCTaacactatgGCTacagaggaaacgaaaagaaatatcgctagac |
| | | | atttagccctatgggattctaaattttttaccgagctagaaaaaaaaatgtagaatatgtagttattatagaaaacg |
| | | | ataacgttattgaggatattacgtttcttcgtcccgtcttgaaggcaatacatgacaagaaaatagatatcctacag |
| | | | atgagagaaattattacaggaaataaagttaaaaccgagcttgtaatagacaaagatcatgccatatttacatata |
| | | | caggagggtatgatgttagcttatccgcctatattattagagttactacggcgctgaacatcgtagatgaaattata |
| | | | aagtctggaggtctatcatcgggattttattttgaaatagccagaatcgaaaacgaaatgaagattaacaggcaa |
| | | | ataatggataactctgccaaatatgtagaacacgatcctcgtcttgttgcagaacaccgcttcgaaaccatgaaa |
| | | | ccaaatttttggtctagaataggaacggtagctgctaaacgttatccaggagttatgtacacgtttactactccatt |
| | | | gatttcattttttggattgtttgatattaatgttataggtttgattgtaattttgtttattatgtttatgctcatctttaatgttaa |
| | | | atctaaactgttatggttccttacaggaacatttgttaccgcatttatctaa |
| 190 | SS + | AA | MGGAAARLGAVILFVVIVGLHGVRGMAAVKTPVIVVPVIDRPPSETFP |
| | H3L (C86A C90A) | | NVHEHINDQKFDDVKDNEVMQEKRDVVIVNDDPDHYKDYVFIQWT |
| | | | GGNIRDDDKYTHFFSGFANTMATEETKRNIARHLALWDSKFFTELEN |
| | | | KNVEYVVIIENDNVIEDITFLRPVLKAIHDKKIDILQMREIITGNKVKTE |
| | | | LVIDKDHAIFTYTGGYDVSLSAYIIRVTTALNIVDEIIKSGGLSSGFYFEI |
| | | | ARIENEMKINRQIMDNSAKYVEHDPRLVAEHRFETMKPNFWSRIGTV |
| | | | AAKRYPGVMYTFTTPLISFFGLFDINVIGLIVILFIMFMLIFNVKSKLLW |
| | | | FLTGTFVTAFI |
| 191 | SS + | NT | atggggggggctgccgccaggttgggggccgtgattttgtttgtcgtcatagtgggcctccatggggtccgcg |
| | H3L (C86A C90A) | | gcatggcggcggtgaaaactcctgttattgttgtgccagttattgatagacctccatcagaaacatttcctaatgtt |
| | | | catgagcatattaatgatcagaagttcgatgatgtgaaggacaatgaagttatgcaagaaaaaagagatgttgtg |
| | | | atagtcaatgatgatccagatcattacaaggattatgtgtttatacaatggactggaggaaatattagagatgatg |
| | | | acaagtatactcacttcttttcagggtttGCTaacactatgGCTacagaggaaacgaaaagaaatatcgcta |
| | | | gacatttagccctatgggattctaaattttttaccgagctagaaaataaaaatgtagaatatgtagttattatagaaa |
| | | | acgataacgttattgaggatattacgtttcttcgtcccgtcttgaaggcaatacatgacaagaaaatagatatccta |
| | | | cagatgagagaaattattacaggaaataaagttaaaaccgagcttgtaatagacaaagatcatgccatatttacat |
| | | | atacaggagggtatgatgttagcttatccgcctatattattagagttactacggcgctgaacatcgtagatgaaatt |
| | | | ataaagtctggaggtctatcatcgggattttattttgaaatagccagaatcgaaaacgaaatgaagattaacagg |
| | | | caaataatggataactctgccaaatatgtagaacacgatcctcgtcttgttgcagaacaccgcttcgaaaccatg |
| | | | aaaccaaatttttggtctagaataggaacggtagctgctaaacgttatccaggagttatgtacacgtttactactcc |
| | | | attgatttcattttttggattgtttgatattaatgttataggtttgattgtaattttgtttattatgtttatgctcatctttaatgt |
| | | | taaatctaaactgttatggttccttacaggaacatttgttaccgcatttatctaa |
| 192 | E8L | AA | MPQQLSPINIETKKAISDARLKTLDIHYNESKPTTIQNTGKLVRINFKG |
| | | | GYISGGFLPNEYVLSTIHIYWGKEDDYGSNHLIDVYKYSGEINLVHWN |
| | | | KKKYSSYEEAKKHDDGIIIIAIFLQVSDHKNVYFQKIVNQLDSIRSANM |
| | | | SAPFDSVFYLDNLLPSTLDYFTYLGTTINHSADAAWIIFPTPINIHSDQL |
| | | | SKFRTLLSSSNHEGKPHYITENYRNPYKLNDDTQVYYSGEIIRAATTSP |
| | | | VRENYFMKWLSDLREACFSYYQKYIEGNKTFAIIAIVFVFILTAILFLM |
| | | | SQRYSREKQN |
| 193 | E8L | NT | atgcctcaacaactatctcctattaatatagaaactaaaaaagcaatttctgacgcgcgattgaagacgttagaca |
| | | | tacattataatgagtcgaaaccaaccactatccagaatactggaaaactagtaaggattaattttaaaggaggata |
| | | | tataagtggagggtttctccccaatgaatatgtgttatcaacaatacatatatattggggaaaggaagacgattat |
| | | | ggatccaatcacttgatagatgtatacaaatactctggagagattaatctggttcattggaataagaaaaaatata |
| | | | gttcttatgaagaggcaaaaaaacacgatgatggaattatcattattgctatattcttacaagtatcagatcataaaa |
| | | | atgtatattttcaaaagatagtcaatcaattggattccattagatccgctaatatgtctgcgccatttgattcagtatttt |
| | | | atctagacaatttgctacctagtacgttggattattttacatatctaggaacaactatcaaccactctgcagacgca |
| | | | gcatggataatttttccaacaccaataaacattcattctgatcaactatctaaattcagaacactattgtcgtgtcta |
| | | | atcatgagggtaaacctcattatataacagagaattatagaaatccgtataaattgaacgacgacacgcaagtat |
| | | | attattctggggagattatacgagctgcaaccacctcaccagtgcgagagaactattttatgaaatggttgtctga |
| | | | tttgagagaggcatgtttttcatattatcaaaaatatatcgaagggaataaaacattcgcaattattgccatagtattc |
| | | | gtatttatactcaccgctattctctttcttatgagtcaacgatattcgcgagaaaaacaaaactag |
| 194 | SS + E8L | AA | MGGAAARLGAVILFVVIVGLHGVRGMPQQLSPINIETKKAISDARLKT |
| | | | LDIHYNESKPTTIQNTGKLVRINFKGGYISGGFLPNEYVLSTIHIYWGK |
| | | | EDDYGSNHLIDVYKYSGEINLVHWNKKKYSSYEEAKKHDDGIIIIAIFL |
| | | | QVSDHKNVYFQKIVNQLDSIRSANMSAPFDSVFYLDNLLPSTLDYFTY |
| | | | LGTTINHSADAAWIIFPTPINIHSDQLSKFRTLLSSSNHEGKPHYITENY |
| | | | RNPYKLNDDTQVYYSGEIIRAATTSPVRENYFMKWLSDLREACFSYY |
| | | | QKYIEGNKTFAIIAIVFVFILTAILFLMSQRYSREKQN |
| 195 | SS + E8L | NT | atggggggggctgccgccaggttgggggccgtgattttgtttgtcgtcatagtgggcctccatggggtccgcg |
| | | | gcatgcctcaacaactatctcctattaatatagaaactaaaaaagcaatttctgacgcgcgattgaagacgttaga |
| | | | catacattataatgagtcgaaaccaaccactatccagaatactggaaaactagtaaggattaattttaaaggagg |
| | | | atatataagtggagggtttctccccaatgaatatgtgttatcaacaatacatatatattggggaaaggaagacgatt |
| | | | atggatccaatcacttgatagatgtatacaaatactctggagagattaatctggttcattggaataagaaaaaatat |
| | | | agttcttatgaagaggcaaaaaaacacgatgatggaattatcattattgctatattcttacaagtatcagatcataaa |
| | | | aatgtatattttcaaaagatagtcaatcaattggattccattagatccgctaatatgtctgcgccatttgattcagtatt |
| | | | ttatctagacaatttgctacctagtacgttggattattttacatatctaggaacaactatcaaccactctgcagacgc |
| | | | agcatggataatttttccaacaccaataaacattcattctgatcaactatctaaattcagaacactattgtcgtcgtct |
| | | | aatcatgagggtaaacctcattatataacagagaattatagaaatccgtataaattgaacgacgacacgcaagta |
| | | | tattattctggggagattatacgagctgcaaccacctcaccagtgcgagagaactattttatgaaatggttgtctg |
| | | | atttgagagaggcatgtttttcatattatcaaaaatatatcgaagggaataaaacattcgcaattattgccatagtatt |
| | | | cgtatttatactcaccgctattctctttcttatgagtcaacgatattcgcgagaaaaacaaaactag |
| 196 | A28L | AA | MANIINLWNGIVPMVQDVNVASITAFKSMIDETWDKKIEANTCISRKH |
| | | | RNIIHEVIRDFMKAYPKMDENRKSPLGAPMQWLTQYYILKNEYHKT |
| | | | MLAYDDGSLNTKFKTLNIYMITNVGQYILYIVFCIISGKNHDGTPYIYD |
| | | | SEITSNDKNLINDRIKYACKQILHGQLTMALRIRNKFMFIGSPMYLWF |
| | | | NVNGSHVYHEIYDRNVGFHNKEIGRLLYAFMYYLSISGRFLNDLALL |
| | | | KFTYLGESWTFSLSVPEYILYGLGYSVFDTIEKFSNDAILVYIRTNNRN |
| | | | GYDYAEFNKKGIVKVTEDKPDNDKRIHAIRLINYSSDVQHIHFGFRNT |
| | | | LIIDNECTNIQSSAENATDIGHYQDSKINIEDDDIIDDDDDDDDDDDDD |
| | | | DYNPKPTPIPDPHPRPPFPRHDYHKRPKLLPVEEPDPVKKDADRIRLD |
| | | | NHILNTLDHNLNSIGHYCCDTVAVDRLEHHIETLGQYTVILARKINMQ |
| | | | TLLFPWPLPTVHQHAIDGSIPPHGRSTIL |
| 197 | A28L | NT | atggcgaacattataaatttatggaacggaattgtaccaatggttcaagatgttaatgttgcgagcattactgcgttt |
| | | | aaatctatgatagatgaaacatgggataaaaaaatcgaagcaaatacatgcatcagtagaaaacatagaaacat |
| | | | tattcacgaagttattagggactttatgaaagcatatcctaaaatggacgagaatagaaaatctccattaggagct |
| | | | ccaatgcaatggctaacacaatattatattttaaagaatgaatatcacaagaccatgctagcatatgatgatggat |
| | | | cattaaatacaaaatttaaaacattaaacatttacatgattactaacgtgggtcaatatattttatatatagtattttgtat |
| | | | aatatctggtaagaatcatgatggtactccttatatatacgattctgaaataacgagcaatgataaaaatcttattaa |
| | | | tgatcgtatcaagtatgcatgtaaacaaatattacacggtcaattaactatggctctgagaattagaaataaattcat |
| | | | gtttataggatcacccatgtatttatggtttaacgtaaacgggtcacatgtatatcacgaaatatatgatcgtaatgt |
| | | | cggttttcataataaagagataggtagactactatacgcattcatgtactatctatctataagcggtagatttttgaat |
| | | | gatttagcactattaaagtttacgtatttaggagaatcctggacatttagtttgagtgttcctgaatatatattatatgg |
| | | | tttaggatattctgtttttgatactattgaaaaatttagcaatgatgctatactcgtttatattagaacaaacaatagaa |
| | | | atggttatgattatgcagagtttaataaaaaaggaattgttaaggtgacagaagataaacccgataacgataagc |
| | | | gaattcatgctataagactcatcaactatagtagtgatgttcaacatatacattttgggtttagaaatacgttaataat |
| | | | agacaatgaatgtactaatattcagtcgagtgctgaaaatgcaactgatataggacattatcaagatagcaaaat |
| | | | aaatatcgaagatgatgatatcatagatgatgatgatgatgatgatgatgatgatgatgatgatgattataatccaa |
| | | | aacctactccgatacctgatcctcatcctagaccaccatttcccagacatgactatcataagaggccgaaacttct |
| | | | tcctgtagaagagcctgatcctgtcaaaaaagacgcggatcgtataagacttgataatcatatattaaacacattg |
| | | | gatcataatcttaattccatcggacattattgttgtgatacagtggcagttgataggttagaacatcacattgaaaca |
| | | | ttgggacaatatacagtaatactagcaagaaagataaatatgcaaacattactgttcccatggccattacctactg |
| | | | tccatcaacatgcgatagatggtagtattccaccacatgggagatctacgatcttataa |
| 198 | SS + A28L | AA | MGGAAARLGAVILFVVIVGLHGVRGMANIINLWNGIVPMVQDVNVA |
| | | | SITAFKSMIDETWDKKIEANTCISRKHRNIIHEVIRDFMKAYPKMDEN |
| | | | RKSPLGAPMQWLTQYYILKNEYHKTMLAYDDGSLNTKFKTLNIYMIT |
| | | | NVGQYILYIVFCIISGKNHDGTPYIYDSEITSNDKNLINDRIKYACKQIL |
| | | | HGQLTMALRIRNKFMFIGSPMYLWFNVNGSHVYHEIYDRNVGFHNK |
| | | | EIGRLLYAFMYYLSISGRFLNDLALLKFTYLGESWTFSLSVPEYILYGL |
| | | | GYSVFDTIEKFSNDAILVYIRTNNRNGYDYAEFNKKGIVKVTEDKPDN |
| | | | DKRIHAIRLINYSSDVQHIHFGFRNTLIIDNECTNIQSSAENATDIGHYQ |
| | | | DSKINIEDDDIIDDDDDDDDDDDDDDYNPKPTPIPDPHPRPPFPRHDYH |
| | | | KRPKLLPVEEPDPVKKDADRIRLDNHILNTLDHNLNSIGHYCCDTVAV |
| | | | DRLEHHIETLGQYTVILARKINMQTLLFPWPLPTVHQHAIDGSIPPHGR |
| | | | STIL |
| 199 | SS + A28L | NT | atggggggggctgccgccaggttgggggccgtgattttgtttgtcgtcatagtgggcctccatggggtccgcg |
| | | | gcatggcgaacattataaatttatggaacggaattgtaccaatggttcaagatgttaatgttgcgagcattactgc |
| | | | gtttaaatctatgatagatgaaacatgggataaaaaaatcgaagcaaatacatgcatcagtagaaaacatagaa |
| | | | acattattcacgaagttattagggactttatgaaagcatatcctaaaatggacgagaatagaaaatctccattagg |
| | | | agctccaatgcaatggctaacacaatattatattttaaagaatgaatatcacaagaccatgctagcatatgatgat |
| | | | ggatcattaaatacaaaatttaaaacattaaacatttacatgattactaacgtgggtcaatatattttatatatagtattt |
| | | | tgtataatatctggtaagaatcatgatggtactccttatatatacgattctgaaataacgagcaatgataaaaatctt |
| | | | attaatgatcgtatcaagtatgcatgtaaacaaatattacacggtcaattaactatggctctgagaattagaaataa |
| | | | attcatgtttataggatcacccatgtatttatggtttaacgtaaacgggtcacatgtatatcacgaaatatatgatcgt |
| | | | aatgtcggttttcataataaagagataggtagactactatacgcattcatgtactatctatctataagcggtagatttt |
| | | | tgaatgatttagcactattaaagtttacgtatttaggagaatcctggacatttagtttgagtgttcctgaatatatattat |
| | | | atggtttaggatattctgtttttgatactattgaaaaatttagcaatgatgctatactcgtttatattagaacaaacaata |
| | | | gaaatggttatgattatgcagagtttaataaaaaaggaattgttaaggtgacagaagataaacccgataacgata |
| | | | agcgaattcatgctataagactcatcaactatagtagtgatgttcaacatatacattttgggtttagaaatacgttaat |
| | | | aatagacaatgaatgtactaatattcagtcgagtgctgaaaatgcaactgatataggacattatcaagatagcaa |
| | | | aataaatatcgaagatgatgatatcatagatgatgatgatgatgatgatgatgatgatgatgatgatgattataatc |
| | | | caaaacctactccgatacctgatcctcatcctagaccaccatttcccagacatgactatcataagaggccgaaac |
| | | | ttcttcctgtagaagagcctgatcctgtcaaaaaagacgcggatcgtataagacttgataatcatatattaaacac |
| | | | attggatcataatcttaattccatcggacattattgttgtgatacagtggcagttgataggttagaacatcacattga |
| | | | aacattgggacaatatacagtaatactagcaagaaagataaatatgcaaacattactgttcccatggccattacct |
| | | | actgtccatcaacatgcgatagatggtagtattccaccacatgggagatctacgatcttataa |
| 200 | A29L | NT | atggggggggctgccgccaggttgggggccgtgattttgtttgtcgtcatagtgggcctccatggggtccgcg |
| | | | gcatggacggaactcttttccccggagatgacgatcttgcaattccagcaactgaatttttctctacaaaggctgc |
| | | | taaaaatccagagactaaacgcgaagcaattgttaaagcctatggagacgataatgaggaaactctcaaacaa |
| | | | cggctaactaatttggaaaaaaagattactaatataacaacaaagtttgaacaaatagaaaagtgttgtaaacgc |
| | | | aacgatgaagttctatttaggttggaaaatcacgctgaaactctaagagcggctatgatatctctggctaaaaag |
| | | | attgatgtacagactggacggcatccatatgagtaa |
| 201 | SS + A29L | NT | atggggggggctgccgccaggttgggggccgtgattttgtttgtcgtcatagtgggcctccatggggtccgcg |
| | | | gcatggggggggctgccgccaggttgggggccgtgattttgtttgtcgtcatagtgggcctccatggggtccg |
| | | | cggcatggacggaactcttttccccggagatgacgatcttgcaattccagcaactgaatttttctctacaaaggct |
| | | | gctaaaaatccagagactaaacgcgaagcaattgttaaagcctatggagacgataatgaggaaactctcaaac |
| | | | aacggctaactaatttggaaaaaaagattactaatataacaacaaagtttgaacaaatagaaaagtgttgtaaac |
| | | | gcaacgatgaagttctatttaggttggaaaatcacgctgaaactctaagagcggctatgatatctctggctaaaa |
| | | | agattgatgtacagactggacggcatccatatgagtaa |
| 202 | IRES | NT | ccccccccccccccccccccctctccctcccccccccctaacgttactggccgaagccgcttggaataaggcc |
| | | | ggtgtgcgtttgtctatatgttattttccaccatattgccgtcttttggcaatgtgagggcccggaaacctggccctg |
| | | | tcttcttgacgagcattcctaggggtctttcccctctcgccaaaggaatgcaaggtctgttgaatgtcgtgaagga |
| | | | agcagttcctctggaagcttcttgaagacaaacaacgtctgtagcgaccctttgcaggcagcggaacccccca |
| | | | cctggcgacaggtgcctctgcggccaaaagccacgtgtataagatacacctgcaaaggcggcacaacccca |
| | | | gtgccacgttgtgagttggatagttgtggaaagagtcaaatggctctcctcaagcgtattcaacaaggggctga |
| | | | aggatgcccagaaggtaccccattgtatgggatctgatctggggcctcggtgcacatgctttacatgtgtttagtc |
| | | | gaggttaaaaaacgtctaggccccccgaaccacggggacgtggttttcctttgaaaaacacgatgataatatgg |
| | | | ccacaacc |
| 203 | Bicistronic | NT | atggggggggctgccgccaggttgggggccgtgattttgtttgtcgtcatagtgggcctccatggggtccgcg |
| | | | gcatggacggaactcttttccccggagatgacgatcttgcaattccagcaactgaatttttctctacaaaggctgc |
| | | | taaaaatccagagactaaacgcgaagcaattgttaaagcctatggagacgataatgaggaaactctcaaacaa |
| | | | cggctaactaatttggaaaaaaagattactaatataacaacaaagtttgaacaaatagaaaagtgttgtaaacgc |
| | | | aacgatgaagttctatttaggttggaaaatcacgctgaaactctaagagcggctatgatatctctggctaaaaag |
| | | | attgatgtacagactggacggcatccatatgagtaaccccccccccccccccccccctctccctccccccccc |
| | | | ctaacgttactggccgaagccgcttggaataaggccggtgtgcgtttgtctatatgttattttccaccatattgccg |
| | | | tcttttggcaatgtgagggcccggaaacctggccctgtcttcttgacgagcattcctaggggtctttcccctctcg |
| | | | ccaaaggaatgcaaggtctgttgaatgtcgtgaaggaagcagttcctctggaagcttcttgaagacaaacaacg |
| | | | tctgtagcgaccctttgcaggcagcggaaccccccacctggcgacaggtgcctctgcggccaaaagccacgt |
| | | | gtataagatacacctgcaaaggcggcacaaccccagtgccacgttgtgagttggatagttgtggaaagagtca |
| | | | aatggctctcctcaagcgtattcaacaaggggctgaaggatgcccagaaggtaccccattgtatgggatctgat |
| | | | ctggggcctcggtgcacatgctttacatgtgtttagtcgaggttaaaaaacgtctaggccccccgaaccacggg |
| | | | gacgtggttttcctttgaaaaacacgatgataatatggccacaaccatggggggggctgccgccaggttgggg |
| | | | gccgtgattttgtttgtcgtcatagtgggcctccatggggtccgcggcatggcgaacattataaatttatggaacg |
| | | | gaattgtaccaatggttcaagatgttaatgttgcgagcattactgcgtttaaatctatgatagatgaaacatgggat |
| | | | aaaaaaatcgaagcaaatacatgcatcagtagaaaacatagaaacattattcacgaagttattagggactttatg |
| | | | aaagcatatcctaaaatggacgagaatagaaaatctccattaggagctccaatgcaatggctaacacaatattat |
| | | | attttaaagaatgaatatcacaagaccatgctagcatatgatgatggatcattaaatacaaaatttaaaacattaaa |
| | | | catttacatgattactaacgtgggtcaatatattttatatatagtattttgtataatatctggtaagaatcatgatggtac |
| | | | tccttatatatacgattctgaaataacgagcaatgataaaaatcttattaatgatcgtatcaagtatgcatgtaaaca |
| | | | aatattacacggtcaattaactatggctctgagaattagaaataaattcatgtttataggatcacccatgtatttatgg |
| | | | tttaacgtaaacgggtcacatgtatatcacgaaatatatgatcgtaatgtcggttttcataataaagagataggtag |
| | | | actactatacgcattcatgtactatctatctataagcggtagatttttgaatgatttagcactattaaagtttacgtattt |
| | | | aggagaatcctggacatttagtttgagtgttcctgaatatatattatatggtttaggatattctgtttttgatactattga |
| | | | aaaatttagcaatgatgctatactcgtttatattagaacaaacaatagaaatggttatgattatgcagagtttaataa |
| | | | aaaaggaattgttaaggtgacagaagataaacccgataacgataagcgaattcatgctataagactcatcaact |
| | | | atagtagtgatgttcaacatatacattttgggtttagaaatacgttaataatagacaatgaatgtactaatattcagtc |
| | | | gagtgctgaaaatgcaactgatataggacattatcaagatagcaaaataaatatcgaagatgatgatatcataga |
| | | | tgatgatgatgatgatgatgatgatgatgatgatgatgattataatccaaaacctactccgatacctgatcctcatc |
| | | | ctagaccaccatttcccagacatgactatcataagaggccgaaacttcttcctgtagaagagcctgatcctgtca |
| | | | aaaaagacgcggatcgtataagacttgataatcatatattaaacacattggatcataatcttaattccatcggacat |
| | | | tattgttgtgatacagtggcagttgataggttagaacatcacattgaaacattgggacaatatacagtaatactagc |
| | | | aagaaagataaatatgcaaacattactgttcccatggccattacctactgtccatcaacatgcgatagatggtagt |
| | | | attccaccacatgggagatctacgatcttataa |
| 204 | A45L fragment | AA | NDFDKVREALDGVNLIIHTAALVDVFGKYTDNEIMKVNYYGTQTILA |
| | | | ACVDLGIKYLIYTSSMEAIGPNKHGNPFIGHEHTLYDISPGHVYAK |
| 205 | A45L fragment | NT | AACGACTTTGATAAGGTGAGAGAAGCGCTAGATGGGGTAAATCTG |
| | | | ATTATTCATACGGCTGCTCTAGTGGATGTATTTGGAAAATACACTG |
| | | | ATAATGAAATCATGAAAGTAAACTATTATGGAACACAGACTATAT |
| | | | TGGCAGCTTGTGTGGACCTAGGAATCAAGTATTTGATCTATACTAG |
| | | | TAGCATGGAAGCAATAGGACCAAATAAACACGGTAATCCATTCAT |
| | | | CGGTCATGAGCATACCCTTTATGATATATCACCAGGACATGTATAC |
| | | | GCAAAA |
| 206 | Q1L fragment | AA | YPMDNVINFINQDVRYSPTFIKTIKDFVNEHLPTMYDGLNDYLHSVIID |
| | | | EDLIEEYKIKSVAMFNLEYKTDIDTLTLDEQIFVEVNISYYDFRYRQFA |
| | | | DEFRDYIMIKERRQITMQSGDRIRRFRRPMSLRSTIIKK |
| 207 | Q1L fragment | NT | TATCCTATGGACAATGTTATAAATTTTATTAATCAAGATGTTAGAT |
| | | | ATAGTCCCACCTTCATCAAGACAATTAAAGATTTTGTCAACGAGCA |
| | | | TCTTCCAACCATGTACGATGGATTAAATGATTATCTACATTCTGTT |
| | | | ATTATCGACGAGGACTTAATAGAGGAATATAAAATTAAATCCGTT |
| | | | GCCATGTTTAATTTGGAATACAAAACTGATATAGATACTCTAACAT |
| | | | TGGACGAACAGATATTTGTAGAGGTAAACATCTCATATTATGATTT |
| | | | TAGATATAGACAATTTGCCGATGAATTTAGAGATTACATCATGATA |
| | | | AAAGAAAGAAGACAAATCACCATGCAATCTGGTGATAGAATAAG |
| | | | AAGGTTTAGACGTCCCATGTCATTGAGATCCACTATCATCAAAAA |
| | | | G |
| 208 | Q1L fragment | AA | TLIPITDSLSFKLFVDTMSVLNDQYAKVVIFFNTIIEYIIATIYYRLTVLD |
| | | | NYTNVKHFVSKVLHTVMEACGVLFSHIKVNDKIEYELEEMVDKGTV |
| | | | PSYLYHLSINVISIIL |
| 209 | Q1L fragment | NT | ACGCTTATTCCAATTACTGATTCTCTTTCATTTAAGCTATTTGTTGA |
| | | | TACCATGTCTGTGTTAAATGATCAATATGCCAAGGTTGTCATCTTC |
| | | | TTCAATACCATCATAGAATATATTATAGCTACTATCTATTATAGAT |
| | | | TGACAGTCTTGGACAATTATACTAATGTCAAACATTTTGTATCCAA |
| | | | AGTGTTACACACTGTCATGGAAGCATGTGGCGTACTGTTTTCACAC |
| | | | ATTAAAGTTAATGACAAAATAGAGTATGAATTGGAGGAGATGGTG |
| | | | GACAAAGGTACTGTACCTTCTTATTTATATCATCTGTCCATCAACG |
| | | | TCATTTCAATAATATTG |
| 210 | B12R fragment | AA | FTSDYPFYVSPTEMVDVSMMSIYGEPFNHASVKESFGNFSIIELPYVGD |
| | | | TSMMVILPNKIDGLESIEQNLTDTNFKKWCNSLKATFIDVHIPKFKVI |
| 211 | B12R fragment | NT | TTTACCAGTGATTATCCCTTTTACGTATCTCCAACGGAAATGGTAG |
| | | | ATGTAAGTATGATGTCTATTTACGGCGAGCCATTTAATCACGCATC |
| | | | TGTAAAAGAATCATTCGGTAACTTTTCAATCATAGAACTGCCATAT |
| | | | GTTGGAGATACTAGTATGATGGTCATTCTTCCAAACAAGATTGATG |
| | | | GATTAGAATCCATAGAACAAAATCTAACAGATACAAATTTTAAGA |
| | | | AATGGTGTAACTCTCTGAAAGCTACGTTTATCGATGTGCACATTCC |
| | | | TAAGTTTAAGGTAATA |
| 212 | C17L fragment | AA | KSSSILARRPTPKKMNIVTDSENRLKKNSYIENTNQGNILMDSIFVSTM |
| | | | PVETLFGSYI |
| 213 | C17L fragment | NT | AAATCCTCTTCCATACTGGCTAGACGTCCTACTCCTAAAAAAATGA |
| | | | ACATAGTTACTGATTCGGAAAATCGTTTAAAGAAGAATAGCTATA |
| | | | TAGAAAATACTAATCAGGGAAACATTCTGATGGATAGCATCTTCG |
| | | | TATCCACCATGCCGGTAGAAACATTGTTTGGATCGTACATA |
| 214 | C17L fragment | AA | QFLPQIGGEAIFLIVSLCPTKKLVETGFVIPEISSNDNAKIAARILSEKRK |
| | | | DIIAHINTLIQYRQQLELAYYNSCMLTEFLHYCNSYADTIKESLLK |
| 215 | C17L fragment | NT | CAATTTCTTCCACAAATAGGAGGAGAAGCTATTTTCTTGATTGTTT |
| | | | CACTTTGTCCTACCAAGAAGTTAGTAGAAACAGGATTCGTCATTCC |
| | | | AGAAATATCGTCAAATGATAACGCCAAAATCGCTGCTCGCATTCT |
| | | | ATCAGAGAAACGCAAGGACATCATTGCGCATATTAATACTCTAAT |
| | | | ACAATACAGGCAACAATTGGAATTGGCTTACTATAATTCCTGCATG |
| | | | TTAACTGAGTTTCTGCATTATTGTAATTCGTATGCTGATACCATCA |
| | | | AAGAATCACTTCTAAAA |
| 216 | I3L fragment | AA | RIPNSLHSLSAETITEKTFKDESIFINKLNGAMVEYVSTGESSILRSIEGE |
| | | | LESLSKRERQLAKAIITPVVFYR |
| 217 | I3L fragment | NT | CGCATTCCAAATTCTTTGCATAGTCTTTCGGCGGAAACTATTACAG |
| | | | AGAAAACATTTAAGGATGAATCCATCTTCATCAACAAATTAAACG |
| | | | GAGCCATGGTAGAATATGTTTCGACTGGAGAATCATCCATTCTCAG |
| | | | ATCTATAGAAGGTGAACTAGAATCACTCAGTAAAAGAGAAAGACA |
| | | | ATTAGCCAAGGCAATTATTACACCTGTAGTTTTCTATAGA |
| 218 | HSV-1 gD signal | AA | MGGAAARLGAVILFVVIVGLHGVRS |
| 219 | MITD | AA | IVGIVAGLAVLAVVVIGAVVATVMCRRKSSGGKGGSYSQAASSDSAQ |
| | | | GSDVSLTA |
| 220 | T cell string | AA | MGGAAARLGAVILFVVIVGLHGVRSNDFDKVREALDGVNLIIHTAAL |
| | construct | | VDVFGKYTDNEIMKVNYYGTQTILAACVDLGIKYLIYTSSMEAIGPN |
| | | | KHGNPFIGHEHTLYDISPGHVYAKGGSGGGGSGGYPMDNVINFINQD |
| | | | VRYSPTFIKTIKDFVNEHLPTMYDGLNDYLHSVIIDEDLIEEYKIKSVA |
| | | | MFNLEYKTDIDTLTLDEQIFVEVNISYYDFRYRQFADEFRDYIMIKERR |
| | | | QITMQSGDRIRRFRRPMSLRSTIIKKGGSGGGGSGGTLIPITDSLSFKLF |
| | | | VDTMSVLNDQYAKVVIFFNTIIEYIIATIYYRLTVLDNYTNVKHFVSK |
| | | | VLHTVMEACGVLFSHIKVNDKIEYELEEMVDKGTVPSYLYHLSINVIS |
| | | | IILGGSGGGGSGGFTSDYPFYVSPTEMVDVSMMSIYGEPFNHASVKES |
| | | | FGNFSIIELPYVGDTSMMVILPNKIDGLESIEQNLTDTNFKKWCNSLKA |
| | | | TFIDVHIPKFKVIGGSGGGGSGGKSSSILARRPTPKKMNIVTDSENRLK |
| | | | KNSYIENTNQGNILMDSIFVSTMPVETLFGSYIGGSGGGGSGGQFLPQI |
| | | | GGEAIFLIVSLCPTKKLVETGFVIPEISSNDNAKIAARILSEKRKDIIAHI |
| | | | NTLIQYRQQLELAYYNSCMLTEFLHYCNSYADTIKESLLKGGSGGGG |
| | | | SGGRIPNSLHSLSAETITEKTFKDESIFINKLNGAMVEYVSTGESSILRSI |
| | | | EGELESLSKRERQLAKAIITPVVFYRGGSGGGGSGGIVGIVAGLAVLA |
| | | | VVVIGAVVATVMCRRKSSGGKGGSYSQAASSDSAQGSDVSLTA |
| 221 | T cell string | AA | MGGAAARLGAVILFVVIVGLHGVRSRIPNSLHSLSAETITEKTFKDESI |
| | construct | | FINKLNGAMVEYVSTGESSILRSIEGELESLSKRERQLAKAIITPVVFYR |
| | | | GGSGGGGSGGQFLPQIGGEAIFLIVSLCPTKKLVETGFVIPEISSNDNAK |
| | | | IAARILSEKRKDIIAHINTLIQYRQQLELAYYNSCMLTEFLHYCNSYAD |
| | | | TIKESLLKGGSGGGGSGGKSSSILARRPTPKKMNIVTDSENRLKKNSYI |
| | | | ENTNQGNILMDSIFVSTMPVETLFGSYIGGSGGGGSGGYPMDNVINFI |
| | | | NQDVRYSPTFIKTIKDFVNEHLPTMYDGLNDYLHSVIIDEDLIEEYKIK |
| | | | SVAMFNLEYKTDIDTLTLDEQIFVEVNISYYDFRYRQFADEFRDYIMI |
| | | | KERRQITMQSGDRIRRFRRPMSLRSTIIKKGGSGGGGSGGFTSDYPFY |
| | | | VSPTEMVDVSMMSIYGEPFNHASVKESFGNFSIIELPYVGDTSMMVIL |
| | | | PNKIDGLESIEQNLTDTNFKKWCNSLKATFIDVHIPKFKVIGGSGGGGS |
| | | | GGNDFDKVREALDGVNLIIHTAALVDVFGKYTDNEIMKVNYYGTQTI |
| | | | LAACVDLGIKYLIYTSSMEAIGPNKHGNPFIGHEHTLYDISPGHVYAK |
| | | | GGSGGGGSGGTLIPITDSLSFKLFVDTMSVLNDQYAKVVIFFNTIIEYII |
| | | | ATIYYRLTVLDNYTNVKHFVSKVLHTVMEACGVLFSHIKVNDKIEYE |
| | | | LEEMVDKGTVPSYLYHLSINVISIILGGSGGGGSGGIVGIVAGLAVLAV |
| | | | VVIGAVVATVMCRRKSSGGKGGSYSQAASSDSAQGSDVSLTA |
| 252 | Linker | AA | GGSGG |
| 253 | Linker | NT | gcggctctggagga |
| 254 | HSV-1 gD | AA | GLIAGAVGGSLLAALVICGIVYWMRRHTQKAPKRIRLPHIR |
| | transmembrane | | |
| | domain (TM) | | |
| 255 | HSV-1 gD | NT | ggcctgatcgccggcgcggtgggcggcagtctcctggcagccctggtcatttgcggaattgtgtactggatgc |
| | transmembrane | | gccgccacactcaaaaagccccaaagcgcatacgcctcccccacatccgg |
| | domain (TM) | | |
| 256 | T4 Fibritin ″foldon″ | AA | GYIPEAPRDGQAYVRKDGEWVLLSTFL |
| | trimerization domain | | |
| 257 | T4 Fibritin ″foldon″ | NT | ggttatattcctgaagctccaagagatgggcaagcttacgttcgtaaagatggcgaatgggtattgctttctacctt |
| | trimerization domain | | ttta |
| 258 | M1R | AA | MGAAASIQTTVNTLSERISSKLEQEANASAQTKCDIEIGNFYIRQNHGC |
| | ECTODOMAIN | | NITVKNMCSADADAQLDAVLSAATETYSGLTPEQKAYVPAMFTAAL |
| | (AA 1-186) | | NIQTSVNTVVRDFENYVKQTCNSSAVVDNKLKIQNVIIDECYGAPGSP |
| | | | TNLEFINTGSSKGNCAIKALMQLTTKATTQIAPRQVAGTGVQF |
| 259 | M1R | NT | ATGGGTGCCGCAGCAAGCATACAGACGACTGTGAATACACTCAGT |
| | ECTODOMAIN | | GAACGTATCTCGTCTAAATTAGAACAAGAAGCGAACGCTAGTGCT |
| | (AA 1-186) | | CAAACAAAATGTGATATAGAAATCGGAAATTTTTATATCCGACAA |
| | | | AACCATGGATGTAACATCACTGTTAAAAATATGTGCTCTGCGGAC |
| | | | GCGGATGCTCAGTTGGATGCTGTGTTATCAGCCGCTACAGAAACA |
| | | | TATAGTGGATTAACACCGGAACAAAAAGCATACGTACCAGCTATG |
| | | | TTTACTGCTGCGTTAAACATTCAGACGAGTGTAAACACTGTTGTTA |
| | | | GAGATTTTGAAAATTATGTGAAACAGACTTGTAATTCTAGCGCTGT |
| | | | TGTCGATAACAAATTAAAGATACAAAACGTAATTATAGATGAATG |
| | | | TTACGGAGCCCCAGGATCTCCAACAAATTTGGAATTTATTAATACA |
| | | | GGATCTAGCAAAGGAAATTGTGCCATTAAGGCGTTGATGCAATTG |
| | | | ACTACTAAGGCCACTACTCAAATAGCACCTAGACAAGTTGCTGGT |
| | | | ACAGGAGTTCAGTTT |
| 260 | H3L | AA | MAAVKTPVIVVPVIDRPPSETFPNVHEHINDQKFDDVKDNEVMQEKR |
| | ECTODOMAIN | | DVVIVNDDPDHYKDYVFIQWTGGNIRDDDKYTHFFSGFCNTMCTEET |
| | (AA 1-282) | | KRNIARHLALWDSKFFTELENKNVEYVVIIENDNVIEDITFLRPVLKAI |
| | | | HDKKIDILQMREIITGNKVKTELVIDKDHAIFTYTGGYDVSLSAYIIRV |
| | | | TTALNIVDEIIKSGGLSSGFYFEIARIENEMKINRQIMDNSAKYVEHDP |
| | | | RLVAEHRFETMKPNFWSRIGTVAAKRYPGVMYTFTTPLISF |
| 261 | H3L | NT | ATGGCGGCGGTGAAAACTCCTGTTATTGTTGTGCCAGTTATTGATA |
| | ECTODOMAIN | | GACCTCCATCAGAAACATTTCCTAATGTTCATGAGCATATTAATGA |
| | (AA 1-282) | | TCAGAAGTTCGATGATGTGAAGGACAATGAAGTTATGCAAGAAAA |
| | | | AAGAGATGTTGTGATAGTCAATGATGATCCAGATCATTACAAGGA |
| | | | TTATGTGTTTATACAATGGACTGGAGGAAATATTAGAGATGATGA |
| | | | CAAGTATACTCACTTCTTTTCAGGGTTTTGTAACACTATGTGTACA |
| | | | GAGGAAACGAAAAGAAATATCGCTAGACATTTAGCCCTATGGGAT |
| | | | TCTAAATTTTTTACCGAGCTAGAAAATAAAAATGTAGAATATGTA |
| | | | GTTATTATAGAAAACGATAACGTTATTGAGGATATTACGTTTCTTC |
| | | | GTCCCGTCTTGAAGGCAATACATGACAAGAAAATAGATATCCTAC |
| | | | AGATGAGAGAAATTATTACAGGAAATAAAGTTAAAACCGAGCTTG |
| | | | TAATAGACAAAGATCATGCCATATTTACATATACAGGAGGGTATG |
| | | | ATGTTAGCTTATCCGCCTATATTATTAGAGTTACTACGGCGCTGAA |
| | | | CATCGTAGATGAAATTATAAAGTCTGGAGGTCTATCATCGGGATTT |
| | | | TATTTTGAAATAGCCAGAATCGAAAACGAAATGAAGATTAACAGG |
| | | | CAAATAATGGATAACTCTGCCAAATATGTAGAACACGATCCTCGT |
| | | | CTTGTTGCAGAACACCGCTTCGAAACCATGAAACCAAATTTTTGGT |
| | | | CTAGAATAGGAACGGTAGCTGCTAAACGTTATCCAGGAGTTATGT |
| | | | ACACGTTTACTACTCCATTGATTTCATTT |
| 262 | H3L | AA | MAAVKTPVIVVPVIDRPPSETFPNVHEHINDQKFDDVKDNEVMQEKR |
| | ECTODOMAIN, | | DVVIVNDDPDHYKDYVFIQWTGGNIRDDDKYTHFFSGFANTMATEE |
| | C86A/C90A (AA 1- | | TKRNIARHLALWDSKFFTELENKNVEYVVIIENDNVIEDITFLRPVLKA |
| | 282) | | IHDKKIDILQMREIITGNKVKTELVIDKDHAIFTYTGGYDVSLSAYIIRV |
| | | | TTALNIVDEIIKSGGLSSGFYFEIARIENEMKINRQIMDNSAKYVEHDP |
| | | | RLVAEHRFETMKPNFWSRIGTVAAKRYPGVMYTFTTPLISF |
| 263 | H3L | NT | ATGGCGGCGGTGAAAACTCCTGTTATTGTTGTGCCAGTTATTGATA |
| | ECTODOMAIN, | | GACCTCCATCAGAAACATTTCCTAATGTTCATGAGCATATTAATGA |
| | C86A/C90A (AA 1- | | TCAGAAGTTCGATGATGTGAAGGACAATGAAGTTATGCAAGAAAA |
| | 282) | | AAGAGATGTTGTGATAGTCAATGATGATCCAGATCATTACAAGGA |
| | | | TTATGTGTTTATACAATGGACTGGAGGAAATATTAGAGATGATGA |
| | | | CAAGTATACTCACTTCTTTTCAGGGTTTGCTAACACTATGGCTACA |
| | | | GAGGAAACGAAAAGAAATATCGCTAGACATTTAGCCCTATGGGAT |
| | | | TCTAAATTTTTTACCGAGCTAGAAAATAAAAATGTAGAATATGTA |
| | | | GTTATTATAGAAAACGATAACGTTATTGAGGATATTACGTTTCTTC |
| | | | GTCCCGTCTTGAAGGCAATACATGACAAGAAAATAGATATCCTAC |
| | | | AGATGAGAGAAATTATTACAGGAAATAAAGTTAAAACCGAGCTTG |
| | | | TAATAGACAAAGATCATGCCATATTTACATATACAGGAGGGTATG |
| | | | ATGTTAGCTTATCCGCCTATATTATTAGAGTTACTACGGCGCTGAA |
| | | | CATCGTAGATGAAATTATAAAGTCTGGAGGTCTATCATCGGGATTT |
| | | | TATTTTGAAATAGCCAGAATCGAAAACGAAATGAAGATTAACAGG |
| | | | CAAATAATGGATAACTCTGCCAAATATGTAGAACACGATCCTCGT |
| | | | CTTGTTGCAGAACACCGCTTCGAAACCATGAAACCAAATTTTTGGT |
| | | | CTAGAATAGGAACGGTAGCTGCTAAACGTTATCCAGGAGTTATGT |
| | | | ACACGTTTACTACTCCATTGATTTCATTT |
| 264 | E8L CARBONIC | AA | MPQQLSPINIETKKAISDARLKTLDIHYNESKPTTIQNTGKLVRINFKG |
| | ANHYDRASE | | GYISGGFLPNEYVLSTIHIYWGKEDDYGSNHLIDVYKYSGEINLVHWN |
| | DOMAIN (AA 1- | | KKKYSSYEEAKKHDDGIIIIAIFLQVSDHKNVYFQKIVNQLDSIRSANM |
| | 234) | | SAPFDSVFYLDNLLPSTLDYFTYLGTTINHSADAAWIIFPTPINIHSDQL |
| | | | SKFRTLLSSSNHEGKPHYITENYRNPYKLNDDTQVYYS |
| 265 | E8L CARBONIC | NT | ATGCCTCAACAACTATCTCCTATTAATATAGAAACTAAAAAAGCA |
| | ANHYDRASE | | ATTTCTGACGCGCGATTGAAGACGTTAGACATACATTATAATGAGT |
| | DOMAIN (AA 1- | | CGAAACCAACCACTATCCAGAATACTGGAAAACTAGTAAGGATTA |
| | 234) | | ATTTTAAAGGAGGATATATAAGTGGAGGGTTTCTCCCCAATGAAT |
| | | | ATGTGTTATCAACAATACATATATATTGGGGAAAGGAAGACGATT |
| | | | ATGGATCCAATCACTTGATAGATGTATACAAATACTCTGGAGAGA |
| | | | TTAATCTGGTTCATTGGAATAAGAAAAAATATAGTTCTTATGAAGA |
| | | | GGCAAAAAAACACGATGATGGAATTATCATTATTGCTATATTCTTA |
| | | | CAAGTATCAGATCATAAAAATGTATATTTTCAAAAGATAGTCAAT |
| | | | CAATTGGATTCCATTAGATCCGCTAATATGTCTGCGCCATTTGATT |
| | | | CAGTATTTTATCTAGACAATTTGCTACCTAGTACGTTGGATTATTTT |
| | | | ACATATCTAGGAACAACTATCAACCACTCTGCAGACGCAGCATGG |
| | | | ATAATTTTTCCAACACCAATAAACATTCATTCTGATCAACTATCTA |
| | | | AATTCAGAACACTATTGTCGTCGTCTAATCATGAGGGTAAACCTCA |
| | | | TTATATAACAGAGAATTATAGAAATCCGTATAAATTGAACGACGA |
| | | | CACGCAAGTATATTATTCT |
| 266 | E8L FULL | AA | MPQQLSPINIETKKAISDARLKTLDIHYNESKPTTIQNTGKLVRINFKG |
| | ECTODOMAIN | | GYISGGFLPNEYVLSTIHIYWGKEDDYGSNHLIDVYKYSGEINLVHWN |
| | (AA 1-275) | | KKKYSSYEEAKKHDDGIIIIAIFLQVSDHKNVYFQKIVNQLDSIRSANM |
| | | | SAPFDSVFYLDNLLPSTLDYFTYLGTTINHSADAAWIIFPTPINIHSDQL |
| | | | SKFRTLLSSSNHEGKPHYITENYRNPYKLNDDTQVYYSGEIIRAATTSP |
| | | | VRENYFMKWLSDLREACFSYYQKYIEGNKT |
| 267 | E8L FULL | NT | ATGCCTCAACAACTATCTCCTATTAATATAGAAACTAAAAAAGCA |
| | ECTODOMAIN | | ATTTCTGACGCGCGATTGAAGACGTTAGACATACATTATAATGAGT |
| | (AA 1-275) | | CGAAACCAACCACTATCCAGAATACTGGAAAACTAGTAAGGATTA |
| | | | ATTTTAAAGGAGGATATATAAGTGGAGGGTTTCTCCCCAATGAAT |
| | | | ATGTGTTATCAACAATACATATATATTGGGGAAAGGAAGACGATT |
| | | | ATGGATCCAATCACTTGATAGATGTATACAAATACTCTGGAGAGA |
| | | | TTAATCTGGTTCATTGGAATAAGAAAAAATATAGTTCTTATGAAGA |
| | | | GGCAAAAAAACACGATGATGGAATTATCATTATTGCTATATTCTTA |
| | | | CAAGTATCAGATCATAAAAATGTATATTTTCAAAAGATAGTCAAT |
| | | | CAATTGGATTCCATTAGATCCGCTAATATGTCTGCGCCATTTGATT |
| | | | CAGTATTTTATCTAGACAATTTGCTACCTAGTACGTTGGATTATTTT |
| | | | ACATATCTAGGAACAACTATCAACCACTCTGCAGACGCAGCATGG |
| | | | ATAATTTTTCCAACACCAATAAACATTCATTCTGATCAACTATCTA |
| | | | AATTCAGAACACTATTGTCGTCGTCTAATCATGAGGGTAAACCTCA |
| | | | TTATATAACAGAGAATTATAGAAATCCGTATAAATTGAACGACGA |
| | | | CACGCAAGTATATTATTCTGGGGAGATTATACGAGCTGCAACCAC |
| | | | CTCACCAGTGCGAGAGAACTATTTTATGAAATGGTTGTCTGATTTG |
| | | | AGAGAGGCATGTTTTTCATATTATCAAAAATATATCGAAGGGAAT |
| | | | AAAACA |
| 268 | SPLIT POLY-A | NT | AAAAAAAAAAAAAAAAAAAAAAAAAAAAAAGCAUAUGACUAAA |
| | TAIL | | AAAAAAAAAAAAAAAAAAAAAAAAAAAAAAAAAAAAAAAAAAA |
| | | | AAAAAAAAAAAAAAAAAAAAAAAA |
| 270 | B5R | AA | MKTISVVTLLCVLPAVVYSTCTVPTMNNAKLTSTETSFNDKQKVTFT |
| | | | CDQGYHSLDPNAVCETDKWKYENPCKKMCTVSDYVSELYDKPLYE |
| | | | VNSTMTLSCNGETKYFRCEEKNGNTSWNDTVTCPNAECQPLQLEHG |
| | | | SCQPVKEKYSFGEYITINCDVGYEVIGASYISCTANSWNVIPSCQQKC |
| | | | DMPSLSNGLISGSTFSIGGVIHLSCKSGFILTGSPSSTCIDGKWNPILPTC |
| | | | VRSNKEFDPVDDGPDDETDLSKLSKDVVQYEQEIESLEATYHIIIVALT |
| | | | IMGVIFLISVIVLVCSCDKNNDQYKFHKLLP |
| 271 | A27L | AA | MDGTLFPGDDDLAIPATEFFSTKAAKKPEAKREAIVKADEDDNEETL |
| | | | KQRLTNLEKKITNVTTKFEQIEKCCKRNDEVLFRLENHAETLRAAMIS |
| | | | LAKKIDVQTGRRPYE |

### EQUIVALENTS

Those skilled in the art will recognize, or be able to ascertain using no more than routine experimentation, many equivalents to the specific embodiments of technologies described herein. The scope of the present disclosure is not intended to be limited to the above Description, but rather is as set forth in the following claims.

1. A composition comprising a polyribonucleotide encoding one or more monkeypox antigens or fragments thereof and a pharmaceutically acceptable carrier.

2. A composition comprising a plurality of polyribonucleotides and a pharmaceutically acceptable carrier, wherein at least one polyribonucleotide of the plurality of polyribonucleotides encodes one or more monkeypox antigens or fragments thereof.

3. The composition of claim 2, wherein at least two polyribonucleotides of the plurality of polyribonucleotides are not the same.

4. The composition of claim 2 or 3, wherein all of the polyribonucleotides of the plurality of polyribonucleotides encode one or more monkeypox antigens or fragments thereof.

5. The composition of any one of claims 2-4, wherein at least one polyribonucleotide of the plurality of polyribonucleotides encodes only one monkeypox antigen or fragment thereof.

6. The composition of any one of claims 2-5, wherein a first polyribonucleotide of the plurality of polyribonucleotides encodes a first set of one or more monkeypox antigens or fragments thereof, wherein the first set of one or more monkeypox antigens or fragments thereof comprise:
(i) B6R or a fragment of B6R,
(ii) M1R or a fragment of M1R,
(iii) A35R or a fragment of A35R,
(iv) H3L or a fragment of H3L, and
(v) E8L or a fragment of E8L, or
(vi) a combination of any thereof;
wherein a second polyribonucleotide of the plurality of polyribonucleotides encodes a second set of one or more monkeypox antigens or fragments thereof,
wherein the first set of one or more monkeypox antigens or fragments thereof and the second set of one or more monkeypox antigens or fragments thereof are different.

7. The composition of any one of claims 2-5, wherein a first polyribonucleotide of the plurality of polyribonucleotides encodes a first set of one or more monkeypox antigens or fragments thereof, wherein the first set of one or more monkeypox antigens or fragments thereof comprise:
(i) A29L or a fragment of A29L,
(ii) A35R or a fragment of A35R,
(iii) B6R or a fragment of B6R,
(iv) M1R or a fragment of M1R,
(v) E8L or a fragment of E8L,
(vi) A28L or a fragment of A28L,
(vii) H3L or a fragment of H3L, or
(viii) a combination of any thereof,
wherein a second polyribonucleotide of the plurality of polyribonucleotides encodes a second set of one or more monkeypox antigens or fragments thereof,
wherein the first set of one or more monkeypox antigens or fragments thereof and the second set of one or more monkeypox antigens or fragments thereof are different.

8. The composition of any one of claims 2-5, wherein a first polyribonucleotide of the plurality of polyribonucleotides encodes a first set of one or more monkeypox antigens or fragments thereof, wherein the first set of one or more monkeypox antigens or fragments thereof comprise:
(i) A29L or a fragment of A29L,
(ii) A35R or a fragment of A35R,
(iii) B6R or a fragment of B6R,
(iv) M1R or a fragment of M1R,
(v) E8L or a fragment of E8L,
(vi) H3L or a fragment of H3L,
(vii) A45L or a fragment of A45L,
(viii) B9R or a fragment of B9R,
(ix) B16R or a fragment of B16R,
(x) C10L or a fragment of C10L,
(xi) C21L or a fragment of C21L,
(xii) E7R or a fragment of E7R,
(xiii) F3L or a fragment of F3L,
(xiv) F4L or a fragment of F4L,
(xv) G6R or a fragment of G6R,
(xvi) H5R or a fragment of H5R,
(xvii) 13L or a fragment of 13L,
(xviii) O2L or a fragment of O2L,
(xix) Q1L or a fragment of Q1L,
(xx) B12R or a fragment of B12R,
(xxi) C17L or a fragment of C17L,
(xxii) A28L, or a fragment of A28L, or
(xxiii) a combination of any thereof,
wherein a second polyribonucleotide of the plurality of polyribonucleotides encodes a second set of one or more monkeypox antigens or fragments thereof,
wherein the first set of one or more monkeypox antigens or fragments thereof and the second set of one or more monkeypox antigens or fragments thereof are different.

9. The composition of any one of claims 2-8, wherein the first set of one or more monkeypox antigens or fragments thereof and the second set of one or more monkeypox antigens or fragments thereof do not include any of the same monkeypox antigens or fragments thereof.

10. The composition of any one of claims 6-9, wherein
the first polyribonucleotide encodes a B6R antigen or fragment thereof, and
the second polyribonucleotide encodes an M1R antigen or fragment thereof.

11. The composition of claim 10, wherein
the first polyribonucleotide encodes a B6R antigen or fragment thereof, comprising an amino acid sequence at least 85%, at least 90%, at least 91%, at least 92%, at least 93%, at least 94%, at least 95%, at least 96%, at least 97%, at least 98%, at least 99%, or 100% identical to any one of SEQ ID NOs: 21-30, 180, and 182, and
the second polyribonucleotide encodes an M1R antigen or fragment thereof, comprising an amino acid sequence at least 85%, at least 90%, at least 91%, at least 92%, at least 93%, at least 94%, at least 95%, at least 96%, at least 97%, at least 98%, at least 99%, or 100% identical to any one of SEQ ID NOs: 31-40, and 158.

12. The composition of claim 10 or 11, further comprising a third polyribonucleotide encoding an A35R antigen or fragment thereof.

13. The composition of claim 12, wherein
the third polyribonucleotide encodes an A35R antigen or fragment thereof, comprising an amino acid sequence at least 85%, at least 90%, at least 91%, at least 92%, at least 93%, at least 94%, at least 95%, at least 96%, at least 97%, at least 98%, at least 99%, or 100% identical to any one of SEQ ID NOs: 11-20, 172 and 174.

14. The composition of claim 12 or 13, further comprising a fourth polyribonucleotide encoding an E8L antigen or fragment thereof.

15. The composition of claim 14, wherein
the fourth polyribonucleotide encodes an E8L antigen or fragment thereof, comprising an amino acid sequence at least 85%, at least 90%, at least 91%, at least 92%, at least 93%, at least 94%, at least 95%, at least 96%, at least 97%, at least 98%, at least 99%, or 100% identical to any one of SEQ ID NOs: 41-50 and 192.

16. The composition of claim 12 or 13, further comprising a fourth polyribonucleotide encoding an H3L antigen or fragment thereof.

17. The composition of claim 16, wherein
the fourth polyribonucleotide encodes an H3L antigen or fragment thereof, comprising an amino acid sequence at least 85%, at least 90%, at least 91%, at least 92%, at least 93%, at least 94%, at least 95%, at least 96%, at least 97%, at least 98%, at least 99%, or 100% identical to any one of SEQ ID NOs: 51-60, 184, 188 and 190.

18. The composition of any one of claims 1-17, wherein the composition further comprises lipid nanoparticles, polyplexes, lipidated polyplexes, or liposomes,
wherein the polyribonucleotide or plurality of polyribonucleotides are fully or partially encapsulated within the lipid nanoparticles, polyplexes, lipidated polyplexes, or liposomes.

19. The composition of any one of claims 1-18, wherein the composition further comprises lipid nanoparticles,
wherein the polyribonucleotide or plurality of polyribonucleotides are fully or partially encapsulated within the lipid nanoparticles.

20. The composition of claim 19, wherein the lipid nanoparticles target liver cells.

21. The composition of claim 19, wherein the lipid nanoparticles target secondary lymphoid organ cells.

22. The composition of claim any one of claims 18-21, wherein the lipid nanoparticles are cationic lipid nanoparticles.

23. The composition of any one of claims 18-22, wherein the lipid nanoparticles each comprise:
(a) a polymer-conjugated lipid;
(b) a cationic lipid; and
(c) one or more neutral lipids.

24. The composition of claim 23, wherein the polymer-conjugated lipid comprises a PEG-conjugated lipid.

25. The composition of claim 23 or 24, wherein the polymer-conjugated lipid comprises 2-[(polyethylene glycol)-2000]—N,N-ditetradecylacetamide.

26. The composition of any one of claims 23-25, wherein the one or more neutral lipids comprise 1,2-Distearoyl-sn-glycero-3-phosphocholine (DPSC).

27. The composition of any one of claims 23-26, wherein the one or more neutral lipids comprise cholesterol.

28. The composition of any one of claims 23-27, wherein the cationic lipid comprises ((3-hydroxypropyl)azanediyl)bis(nonane-9,1-diyl)bis(2-butyloctanoate).

29. The composition of any one of claims 23-28, wherein the lipid nanoparticles each comprise:
(a) 2-[(polyethylene glycol)-2000]—N,N-ditetradecylacetamide;
(b) DPSC;
(c) cholesterol; and
(d) ((3-hydroxypropyl)azanediyl)bis(nonane-9,1-diyl) bis(2-butyloctanoate).

30. The composition of any one of claims 23-29, wherein the lipid nanoparticles comprise:
(a) the polymer-conjugated lipid at about 1-2.5 mol % of the total lipids;
(b) the cationic lipid at 35-65 mol % of the total lipids; and
(c) the one or more neutral lipids are present in 35-65 mol % of the total lipids.

31. The composition of any one of claims 23-30, wherein the lipid nanoparticles have an average diameter of about 50-150 nm.

32. A method of inducing an immune response against an orthopoxvirus in a subject comprising administering to the subject a composition of any one of claims 1-31.

33. The method of claim 32, wherein the orthopoxvirus is a monkeypox virus, a variola virus, a vaccinia virus, or a cowpox virus.
SEQ ID NO: 160


SEQUENCE LISTING (506 sequences)

SEQ ID NO: 1 - protein, 110 aa, synthetic construct
MDGTLFPGDDDLAIPATEFFSTKAAKNPETKREAIVKAYGDDNEETLKQRLTNLEKKITNITTKFEQIEKCCKRNDEVLFRLENHAETLRAAMISLAKKIDVQTGRHPYE

SEQ ID NO: 2 - protein, 110 aa, synthetic construct
  VARIANT 57: note = Any amino acid
MDGTLFPGDDDLAIPATEFFSTKAAKNPETKREAIVKAYGDDNEETLKQRLTNLEKXITNITTKFEQIEKCCKRNDEVLFRLENHAETLRAAMISLAKKIDVQTGRHPYE

SEQ ID NO: 3 - protein, 110 aa, synthetic construct; identical to SEQ ID NO: 1

SEQ ID NO: 4 - protein, 110 aa, synthetic construct; identical to SEQ ID NO: 1

SEQ ID NO: 5 - protein, 110 aa, synthetic construct; identical to SEQ ID NO: 1

SEQ ID NO: 6 - protein, 110 aa, synthetic construct; identical to SEQ ID NO: 1

SEQ ID NO: 7 - protein, 110 aa, synthetic construct; identical to SEQ ID NO: 1

SEQ ID NO: 8 - protein, 110 aa, synthetic construct
MDGTLFPGDDDLAIPATEFFSTKAAKNPETKREAIVKAYGDDNEETLKQRLTNLEKKITNITTKFEQIEKCCKHNDEVLFRLENHAETLRAAMISLAKKIDVQTGRRPYE

SEQ ID NO: 9 - protein, 110 aa, synthetic construct; identical to SEQ ID NO: 8

SEQ ID NO: 10 - protein, 110 aa, synthetic construct; identical to SEQ ID NO: 8

SEQ ID NO: 11 - protein, 181 aa, synthetic construct
MMTPENDEEQTSVFSATVYGDKIQGKNKRKRVIGLCIRISMVISLLSMITMSAFLIVRLNQCMSANKAAITDSAVAVAAASSTHRKVVSSTTQYDHKESCNGLYYQGSCYILHSDYKSFEDAKANCAAESSTLPNKSDVLTTWLIDYVEDTWGSDGNPITKTTSDYQDSDVSQEVRKYFCT

SEQ ID NO: 12 - protein, 181 aa, synthetic construct; identical to SEQ ID NO: 11

SEQ ID NO: 13 - protein, 181 aa, synthetic construct; identical to SEQ ID NO: 11

SEQ ID NO: 14 - protein, 180 aa, synthetic construct
MTPENDEEQTSVFSATVYGDKIQGKNKRKRVIGLCIRISMVISLLSMITMSAFLIVRLNQCMSANKAAITDSAVAVAAASSTHRKVVSSTTQYDHKESCNGLYYQGSCYILHSDYKSFEDAKANCAAESSTLPNKSDVLTTWLIDYVEDTWGSDGNPITKTTSDYQDSDVSQEVRKYFCT

SEQ ID NO: 15 - protein, 180 aa, synthetic construct; identical to SEQ ID NO: 14

SEQ ID NO: 16 - protein, 181 aa, synthetic construct; identical to SEQ ID NO: 11

SEQ ID NO: 17 - protein, 181 aa, synthetic construct
MMTPENDEEQTSVFSATVYGDKIQGKNKRKRVIGLCIRISMVISLLSMITMSAFLIVRLNQCMSANEAAITDSAVAVAAASSTHRKVASSTTQYDHKESCNGLYYQGSCYILHSDYKSFEDAKANCAAESSTLPNKSDVLTTWLIDYVEDTWGSDGNPITKTTSDYQDSDVSQEVRKYFCT

SEQ ID NO: 18 - protein, 181 aa, synthetic construct; identical to SEQ ID NO: 17

SEQ ID NO: 19 - protein, 181 aa, synthetic construct; identical to SEQ ID NO: 17

SEQ ID NO: 20 - protein, 181 aa, synthetic construct; identical to SEQ ID NO: 17

SEQ ID NO: 21 - protein, 317 aa, synthetic construct
MKTISVVTLLCVLPAVVYSTCTVPTMNNAKLTSTETSFNDKQKVTFTCDSGYHSLDPNAVCETDKWKYENPCKKMCTVSDYVSELYDKPLYEVNSTMTLSCNGETKYFRCEEKNGNTSWNDTVTCPNAECQPLQLEHGSCQPVKEKYSFGEYMTINCDVGYEVIGVSYISCTANSWNVIPSCQQKCDIPSLSNGLISGSTFSIGGVIHLSCKSGFTLTGSPSSTCIDGKWNPILPTCVRSNEEFDPVDDGPDDETDLSKLSKDVVQYEQEIESLEATYHIIIMALTIMGVIFLISIIVLVCSCDKNNDQYKFHKLLP

SEQ ID NO: 22 - protein, 317 aa, synthetic construct
  VARIANT 1: note = Any amino acid
  VARIANT 2: note = Any amino acid
  VARIANT 3: note = Any amino acid
  VARIANT 4: note = Any amino acid
  VARIANT 5: note = Any amino acid
  VARIANT 6: note = Any amino acid
  VARIANT 7: note = Any amino acid
  VARIANT 8: note = Any amino acid
  VARIANT 9: note = Any amino acid
  VARIANT 10: note = Any amino acid
  VARIANT 11: note = Any amino acid
  VARIANT 12: note = Any amino acid
  VARIANT 13: note = Any amino acid
  VARIANT 14: note = Any amino acid
  VARIANT 15: note = Any amino acid
  VARIANT 16: note = Any amino acid
  VARIANT 17: note = Any amino acid
  VARIANT 18: note = Any amino acid
  VARIANT 19: note = Any amino acid
  VARIANT 20: note = Any amino acid
  VARIANT 21: note = Any amino acid
  VARIANT 22: note = Any amino acid
  VARIANT 23: note = Any amino acid
  VARIANT 24: note = Any amino acid
  VARIANT 25: note = Any amino acid
  VARIANT 26: note = Any amino acid
  VARIANT 27: note = Any amino acid
  VARIANT 28: note = Any amino acid
  VARIANT 29: note = Any amino acid
  VARIANT 30: note = Any amino acid
  VARIANT 31: note = Any amino acid
  VARIANT 32: note = Any amino acid
  VARIANT 33: note = Any amino acid
  VARIANT 34: note = Any amino acid
  VARIANT 35: note = Any amino acid
  VARIANT 36: note = Any amino acid
  VARIANT 37: note = Any amino acid
  VARIANT 38: note = Any amino acid
  VARIANT 39: note = Any amino acid
  VARIANT 40: note = Any amino acid
  VARIANT 41: note = Any amino acid
  VARIANT 42: note = Any amino acid
  VARIANT 43: note = Any amino acid
  VARIANT 44: note = Any amino acid
  VARIANT 45: note = Any amino acid
  VARIANT 46: note = Any amino acid
  VARIANT 47: note = Any amino acid
  VARIANT 48: note = Any amino acid
  VARIANT 49: note = Any amino acid
  VARIANT 50: note = Any amino acid
  VARIANT 51: note = Any amino acid
  VARIANT 52: note = Any amino acid
  VARIANT 53: note = Any amino acid
  VARIANT 54: note = Any amino acid
  VARIANT 55: note = Any amino acid
  VARIANT 56: note = Any amino acid
  VARIANT 57: note = Any amino acid
  VARIANT 58: note = Any amino acid
  VARIANT 59: note = Any amino acid
  VARIANT 60: note = Any amino acid
  VARIANT 61: note = Any amino acid
  VARIANT 62: note = Any amino acid
  VARIANT 63: note = Any amino acid
  VARIANT 64: note = Any amino acid
  VARIANT 65: note = Any amino acid
  VARIANT 66: note = Any amino acid
  VARIANT 67: note = Any amino acid
  VARIANT 68: note = Any amino acid
  VARIANT 69: note = Any amino acid
  VARIANT 70: note = Any amino acid
  VARIANT 71: note = Any amino acid
  VARIANT 72: note = Any amino acid
  VARIANT 73: note = Any amino acid
  VARIANT 74: note = Any amino acid
  VARIANT 75: note = Any amino acid
  VARIANT 76: note = Any amino acid
  VARIANT 77: note = Any amino acid
  VARIANT 78: note = Any amino acid
  VARIANT 79: note = Any amino acid
  VARIANT 80: note = Any amino acid
  VARIANT 81: note = Any amino acid
  VARIANT 82: note = Any amino acid
  VARIANT 83: note = Any amino acid
  VARIANT 84: note = Any amino acid
  VARIANT 85: note = Any amino acid
  VARIANT 86: note = Any amino acid
  VARIANT 87: note = Any amino acid
  VARIANT 88: note = Any amino acid
  VARIANT 89: note = Any amino acid
  VARIANT 90: note = Any amino acid
  VARIANT 91: note = Any amino acid
  VARIANT 92: note = Any amino acid
  VARIANT 93: note = Any amino acid
  VARIANT 94: note = Any amino acid
  VARIANT 95: note = Any amino acid
  VARIANT 96: note = Any amino acid
  VARIANT 97: note = Any amino acid
  VARIANT 98: note = Any amino acid
  VARIANT 99: note = Any amino acid
  VARIANT 100: note = Any amino acid
  VARIANT 101: note = Any amino acid
  VARIANT 102: note = Any amino acid
  VARIANT 103: note = Any amino acid
  VARIANT 104: note = Any amino acid
  VARIANT 105: note = Any amino acid
  VARIANT 106: note = Any amino acid
  VARIANT 107: note = Any amino acid
  VARIANT 108: note = Any amino acid
  VARIANT 109: note = Any amino acid
  VARIANT 110: note = Any amino acid
  VARIANT 111: note = Any amino acid
  VARIANT 112: note = Any amino acid
  VARIANT 113: note = Any amino acid
  VARIANT 114: note = Any amino acid
  VARIANT 115: note = Any amino acid
  VARIANT 116: note = Any amino acid
  VARIANT 117: note = Any amino acid
  VARIANT 118: note = Any amino acid
  VARIANT 119: note = Any amino acid
  VARIANT 120: note = Any amino acid
  VARIANT 121: note = Any amino acid
  VARIANT 122: note = Any amino acid
  VARIANT 123: note = Any amino acid
  VARIANT 124: note = Any amino acid
  VARIANT 125: note = Any amino acid
  VARIANT 126: note = Any amino acid
  VARIANT 127: note = Any amino acid
  VARIANT 128: note = Any amino acid
  VARIANT 129: note = Any amino acid
  VARIANT 130: note = Any amino acid
  VARIANT 131: note = Any amino acid
  VARIANT 132: note = Any amino acid
  VARIANT 133: note = Any amino acid
  VARIANT 134: note = Any amino acid
  VARIANT 135: note = Any amino acid
  VARIANT 136: note = Any amino acid
  VARIANT 137: note = Any amino acid
  VARIANT 138: note = Any amino acid
  VARIANT 139: note = Any amino acid
  VARIANT 140: note = Any amino acid
  VARIANT 141: note = Any amino acid
  VARIANT 142: note = Any amino acid
  VARIANT 143: note = Any amino acid
  VARIANT 144: note = Any amino acid
  VARIANT 145: note = Any amino acid
  VARIANT 146: note = Any amino acid
  VARIANT 147: note = Any amino acid
  VARIANT 148: note = Any amino acid
  VARIANT 149: note = Any amino acid
  VARIANT 150: note = Any amino acid
  VARIANT 151: note = Any amino acid
  VARIANT 152: note = Any amino acid
  VARIANT 153: note = Any amino acid
  VARIANT 154: note = Any amino acid
  VARIANT 155: note = Any amino acid
  VARIANT 156: note = Any amino acid
  VARIANT 157: note = Any amino acid
  VARIANT 158: note = Any amino acid
  VARIANT 159: note = Any amino acid
  VARIANT 160: note = Any amino acid
  VARIANT 161: note = Any amino acid
  VARIANT 162: note = Any amino acid
  VARIANT 163: note = Any amino acid
  VARIANT 164: note = Any amino acid
  VARIANT 165: note = Any amino acid
  VARIANT 166: note = Any amino acid
  VARIANT 167: note = Any amino acid
  VARIANT 168: note = Any amino acid
  VARIANT 169: note = Any amino acid
  VARIANT 170: note = Any amino acid
  VARIANT 171: note = Any amino acid
  VARIANT 172: note = Any amino acid
  VARIANT 173: note = Any amino acid
  VARIANT 174: note = Any amino acid
  VARIANT 175: note = Any amino acid
  VARIANT 176: note = Any amino acid
  VARIANT 177: note = Any amino acid
  VARIANT 178: note = Any amino acid
  VARIANT 179: note = Any amino acid
  VARIANT 180: note = Any amino acid
  VARIANT 181: note = Any amino acid
  VARIANT 182: note = Any amino acid
  VARIANT 183: note = Any amino acid
  VARIANT 184: note = Any amino acid
  VARIANT 185: note = Any amino acid
  VARIANT 186: note = Any amino acid
  VARIANT 187: note = Any amino acid
  VARIANT 188: note = Any amino acid
  VARIANT 189: note = Any amino acid
  VARIANT 190: note = Any amino acid
  VARIANT 191: note = Any amino acid
  VARIANT 192: note = Any amino acid
  VARIANT 193: note = Any amino acid
  VARIANT 194: note = Any amino acid
  VARIANT 195: note = Any amino acid
  VARIANT 196: note = Any amino acid
  VARIANT 197: note = Any amino acid
  VARIANT 198: note = Any amino acid
  VARIANT 199: note = Any amino acid
  VARIANT 200: note = Any amino acid
  VARIANT 201: note = Any amino acid
  VARIANT 202: note = Any amino acid
  VARIANT 203: note = Any amino acid
  VARIANT 204: note = Any amino acid
  VARIANT 205: note = Any amino acid
  VARIANT 206: note = Any amino acid
  VARIANT 207: note = Any amino acid
  VARIANT 208: note = Any amino acid
  VARIANT 209: note = Any amino acid
  VARIANT 210: note = Any amino acid
  VARIANT 211: note = Any amino acid
  VARIANT 212: note = Any amino acid
  VARIANT 213: note = Any amino acid
  VARIANT 214: note = Any amino acid
  VARIANT 215: note = Any amino acid
  VARIANT 216: note = Any amino acid
  VARIANT 217: note = Any amino acid
  VARIANT 218: note = Any amino acid
  VARIANT 219: note = Any amino acid
  VARIANT 220: note = Any amino acid
  VARIANT 221: note = Any amino acid
  VARIANT 222: note = Any amino acid
  VARIANT 223: note = Any amino acid
  VARIANT 224: note = Any amino acid
  VARIANT 225: note = Any amino acid
  VARIANT 226: note = Any amino acid
  VARIANT 227: note = Any amino acid
  VARIANT 228: note = Any amino acid
  VARIANT 229: note = Any amino acid
  VARIANT 230: note = Any amino acid
  VARIANT 231: note = Any amino acid
  VARIANT 232: note = Any amino acid
  VARIANT 233: note = Any amino acid
  VARIANT 234: note = Any amino acid
  VARIANT 237: note = Any amino acid
  VARIANT 251: note = Any amino acid
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXPTXVRSNEEFDPVDDGXDDETDLSKLSKDVVQYEQEIESLEATYHIIIMALTIMGVIFLISIIVLVCSCDKNNDQYKFHKLLP

SEQ ID NO: 23 - protein, 317 aa, synthetic construct
  VARIANT 313: note = Any amino acid
  VARIANT 314: note = Any amino acid
  VARIANT 315: note = Any amino acid
  VARIANT 316: note = Any amino acid
MKTISVVTLLCVLPAVVYSTCTVPTMNNAKLTSTETSFNDKQKVTFTCDSGYHSLDPNAVCETDKWKYENPCKKMCTVSDYVSELYDKPLYEVNSTMTLSCNGETKYFRCEEKNGNTSWNDTVTCPNAECQPLQLEHGSCQPVKEKYSFGEYMTINCDVGYEVIGVSYISCTANSWNVIPSCQQKCDIPSLSNGLISGSTFSIGGVIHLSCKSGFTLTGSPSSTCIDGKWNPILPTCVRSNEEFDPVDDGPDDETDLSKLSKDVVQYEQEIESLEATYHIIIMALTIMGVIFLISIIVLVCSCDKNNDQYKFXXXXP

SEQ ID NO: 24 - protein, 317 aa, synthetic construct; identical to SEQ ID NO: 21

SEQ ID NO: 25 - protein, 317 aa, synthetic construct; identical to SEQ ID NO: 21

SEQ ID NO: 26 - protein, 317 aa, synthetic construct; identical to SEQ ID NO: 23
  VARIANT 313: note = Any amino acid
  VARIANT 314: note = Any amino acid
  VARIANT 315: note = Any amino acid
  VARIANT 316: note = Any amino acid

SEQ ID NO: 27 - protein, 316 aa, synthetic construct
MKTISVVTLLCVLPAVVYSTCTVPTMNNAKLTSTETSFNDKQKVTFTCDSGYHSLDPNAVCETDKWKYENPCKKMCTVSDYVSELYDKPLYEVNSTMTLSCNGETKYFRCEEKNGNTSWNDTVTCPNAECQPLQLEHGSCQPVKEKYSFGEYMTINCDVGYEVIGVSYISCTANSWNVIPSCQQKCDIPSLSNGLISGSTFSIGGVIHLSCKSGFTLTGSPSSTCIDGKWNPILPTCVRSNEEFDPVDDGPDDETDLSKLSKDVVQYEQEIESLEATYHIIIMALTIMGVIFLISIIVLVCSCDKNNDQYKFHKLL

SEQ ID NO: 28 - protein, 317 aa, synthetic construct; identical to SEQ ID NO: 21

SEQ ID NO: 29 - protein, 317 aa, synthetic construct; identical to SEQ ID NO: 21

SEQ ID NO: 30 - protein, 317 aa, synthetic construct; identical to SEQ ID NO: 21

SEQ ID NO: 31 - protein, 250 aa, synthetic construct
MGAAASIQTTVNTLSERISSKLEQEANASAQTKCDIEIGNFYIRQNHGCNITVKNMCSADADAQLDAVLSAATETYSGLTPEQKAYVPAMFTAALNIQTSVNTVVRDFENYVKQTCNSSAVVDNKLKIQNVIIDECYGAPGSPTNLEFINTGSSKGNCAIKALMQLTTKATTQIAPRQVAGTGVQFYMIVIGVIILAALFMYYAKRMLFTSTNDKIKLILANKENVHWTTYMDTFFRTSPMIIATTDIQN

SEQ ID NO: 32 - protein, 250 aa, synthetic construct
  VARIANT 2: note = Any amino acid
  VARIANT 5: note = Any amino acid
  VARIANT 109: note = Any amino acid
  VARIANT 221: note = Any amino acid
MXAAXSIQTTVNTLSERISSKLEQEANASAQTKCDIEIGNFYIRQNHGCNITVKNMCSADADAQLDAVLSAATETYSGLTPEQKAYVPAMFTAALNIQTSVNTVVRDFXNYVKQTCNSSAVVDNKLKIQNVIIDECYGAPGSPTNLEFINTGSSKGNCAIKALMQLTTKATTQIAPRQVAGTGVQFYMIVIGVIILAALFMYYAKRMLFTSTNDKIKLILXNKENVHWTTYMDTFFRTSPMIIATTDIQN

SEQ ID NO: 33 - protein, 250 aa, synthetic construct; identical to SEQ ID NO: 31

SEQ ID NO: 34 - protein, 250 aa, synthetic construct; identical to SEQ ID NO: 31

SEQ ID NO: 35 - protein, 250 aa, synthetic construct; identical to SEQ ID NO: 31

SEQ ID NO: 36 - protein, 250 aa, synthetic construct; identical to SEQ ID NO: 31

SEQ ID NO: 37 - protein, 250 aa, synthetic construct; identical to SEQ ID NO: 31

SEQ ID NO: 38 - protein, 250 aa, synthetic construct; identical to SEQ ID NO: 31

SEQ ID NO: 39 - protein, 250 aa, synthetic construct; identical to SEQ ID NO: 31

SEQ ID NO: 40 - protein, 250 aa, synthetic construct
MGAAASIQTTVNTLSERISSKLEQEANASAQTKCDIEIGNFYIRQNHGCNITVKNMCSADADAQLDAVLSAATETYSGLTPEQKAYVPAMFTAALNIQTSVNTVVRDFENYVKQTCNSSAVVDNKLKIQNVIIDECYGAPGSPTNLEFINTGSSKGNCAIKALMQLTTKATTQIAPRQVAGTGVQFYMIVIGVIILAALFMYYAKRILFTSTNDKIKLILANKENVHWTTYMDTFFRTSPMIIATTDIQN

SEQ ID NO: 41 - protein, 304 aa, synthetic construct
MPQQLSPINIETKKAISDARLKTLDIHYNESKPTTIQNTGKLVRINFKGGYISGGFLPNEYVLSTIHIYWGKEDDYGSNHLIDVYKYSGEINLVHWNKKKYSSYEEAKKHDDGIIIIAIFLQVSDHKNVYFQKIVNQLDSIRSANMSAPFDSVFYLDNLLPSTLDYFTYLGTTINHSADAAWIIFPTPINIHSDQLSKFRTLLSSSNHEGKPHYITENYRNPYKLNDDTQVYYSGEIIRAATTSPVRENYFMKWLSDLREACFSYYQKYIEGNKTFAIIAIVFVFILTAILFLMSQRYSREKQN

SEQ ID NO: 42 - protein, 304 aa, synthetic construct
  VARIANT 1: note = Any amino acid
  VARIANT 37: note = Any amino acid
  VARIANT 47: note = Any amino acid
  VARIANT 109: note = Any amino acid
  VARIANT 128: note = Any amino acid
  VARIANT 184: note = Any amino acid
  VARIANT 185: note = Any amino acid
  VARIANT 186: note = Any amino acid
  VARIANT 200: note = Any amino acid
  VARIANT 217: note = Any amino acid
  VARIANT 236: note = Any amino acid
  VARIANT 238: note = Any amino acid
  VARIANT 239: note = Any amino acid
  VARIANT 260: note = Any amino acid
  VARIANT 264: note = Any amino acid
  VARIANT 294: note = Any amino acid
  VARIANT 301: note = Any amino acid
XPQQLSPINIETKKAISDARLKTLDIHYNESKPTTIXNTGKLVRINXKGGYISGGFLPNEYVLSTIHIYWGKEDDYGSNHLIDVYKYSGEINLVHWNKKKYSSYEEAKXHDDGIIIIAIFLQVSDHKXVYFQKIVNQLDSIRSANMSAPFDSVFYLDNLLPSTLDYFTYLGTTINHSADAAWIXXXTPINIHSDQLSKFXTLLSSSNHEGKPHYITXNYRNPYKLNDDTQVYYSGXIXXAATTSPVRENYFMKWLSDLRXACFXYYQKYIEGNKTFAIIAIVFVFILTAILFLXSQRYSRXKQN

SEQ ID NO: 43 - protein, 304 aa, synthetic construct; identical to SEQ ID NO: 41

SEQ ID NO: 44 - protein, 304 aa, synthetic construct; identical to SEQ ID NO: 41

SEQ ID NO: 45 - protein, 304 aa, synthetic construct; identical to SEQ ID NO: 41

SEQ ID NO: 46 - protein, 304 aa, synthetic construct; identical to SEQ ID NO: 41

SEQ ID NO: 47 - protein, 304 aa, synthetic construct; identical to SEQ ID NO: 41

SEQ ID NO: 48 - protein, 304 aa, synthetic construct
MPQQLSPINIETKKAISDTRLKTLDIHYNESKPTTIQNTGKLVRINFKGGYISGGFLPNEYVLSTIHIYWGKEDDYGSNHLIDVYKYSGEINLVHWNKKKYSSYEEAKKHDDGIIIIAIFLQVSDHKNVYFQKIVNQLDSIRSANMSAPFDSVFYLDNLLPSTLDYFTYLGTTINHSADAAWIIFPTPINIHSDQLSKFRTLLSSSNHEGKPHYITENYRNPYKLNDDTQVYYSGEIIRAATTSPVRENYFMKWLSDLREACFSYYQKYIEGNKTFAIIAIVFVFILTAILFLMSQRYSREKQN

SEQ ID NO: 49 - protein, 304 aa, synthetic construct; identical to SEQ ID NO: 48

SEQ ID NO: 50 - protein, 304 aa, synthetic construct
MPQQLSPINIETKKAISDTRLKTLDIHYNESKPTTIQNTGKLVRINFKGGYISGGFLPNEYVLSTIHIYWGKEDDYGSNHLIDVYKYSGEINLVHWNKKKYSSYEEAKKHDDGIIIIAIFLQVSDHKNVYFQKIVNQLDSIRSANMSAPFDSVFYLDNLLPSTLDYFTYLGTTINHSADAAWIIFPTPINIHSDQLSKFRTLLSSSNHEGKPHYITENYRNPYKLNDDTQVYYSGEIIRAATTSPVRENYFMKWLSDLREACFSYYQKYIEGNKTFAIIAIVFVFILTTILFLMSQRYSREKQN

SEQ ID NO: 51 - protein, 324 aa, synthetic construct
MAAVKTPVIVVPVIDRPPSETFPNVHEHINDQKFDDVKDNEVMQEKRDVVIVNDDPDHYKDYVFIQWTGGNIRDDDKYTHFFSGFCNTMCTEETKRNIARHLALWDSKFFTELENKNVEYVVIIENDNVIEDITFLRPVLKAIHDKKIDILQMREIITGNKVKTELVIDKDHAIFTYTGGYDVSLSAYIIRVTTALNIVDEIIKSGGLSSGFYFEIARIENEMKINRQIMDNSAKYVEHDPRLVAEHRFETMKPNFWSRIGTVAAKRYPGVMYTFTTPLISFFGLFDINVIGLIVILFIMFMLIFNVKSKLLWFLTGTFVTAFI

SEQ ID NO: 52 - protein, 324 aa, synthetic construct
  VARIANT 40: note = Any amino acid
  VARIANT 110: note = Any amino acid
  VARIANT 168: note = Any amino acid
  VARIANT 191: note = Any amino acid
MAAVKTPVIVVPVIDRPPSETFPNVHEHINDQKFDDVKDXEVMQEKRDVVIVNDDPDHYKDYVFIQWTGGNIRDDDKYTHFFSGFCNTMCTEETKRNIARHLALWDSKFXTELENKNVEYVVIIENDNVIEDITFLRPVLKAIHDKKIDILQMREIITGNKVKTELVXDKDHAIFTYTGGYDVSLSAYIIXVTTALNIVDEIIKSGGLSSGFYFEIARIENEMKINRQIMDNSAKYVEHDPRLVAEHRFETMKPNFWSRIGTVAAKRYPGVMYTFTTPLISFFGLFDINVIGLIVILFIMFMLIFNVKSKLLWFLTGTFVTAFI

SEQ ID NO: 53 - protein, 324 aa, synthetic construct; identical to SEQ ID NO: 51

SEQ ID NO: 54 - protein, 324 aa, synthetic construct; identical to SEQ ID NO: 51

SEQ ID NO: 55 - protein, 324 aa, synthetic construct; identical to SEQ ID NO: 51

SEQ ID NO: 56 - protein, 324 aa, synthetic construct; identical to SEQ ID NO: 51

SEQ ID NO: 57 - protein, 324 aa, synthetic construct; identical to SEQ ID NO: 51

SEQ ID NO: 58 - protein, 324 aa, synthetic construct
MAAAKTPVIVVPVIDRPPSETFPNVHEHINDQKFDDVKDNEVMQEKRDVVIVNDDPDHYKDYVFIQWTGGNIRDDDKYTHFFSGFCNTMCTEETKRNIARHLALWDSKFFIELENKNVEYVVIIENDNVIEDITFLRPVLKAIHDKKIDILQMREIITGNKVKTELVIDKDHAIFTYTGGYDVSLSAYIIRVTTALNIVDEIIKSGGLSSGFYFEIARIENEMKINRQIMDNSAKYVEHDPRLVAEHRFETMKPNFWSRIGTVAAKRYPGVMYTFTTPLISFFGLFDINVIGLIVILFIMFMLIFNVKSKLLWFLTGTFVTAFI

SEQ ID NO: 59 - protein, 324 aa, synthetic construct; identical to SEQ ID NO: 58

SEQ ID NO: 60 - protein, 324 aa, synthetic construct; identical to SEQ ID NO: 58

SEQ ID NO: 61 - protein, 346 aa, synthetic construct
MAVYAVTGGAGFLGRYIVKLLISADDVQEIRVIDIVEDPQPITSKVKVINYIQCDINDFDKVREALDGVNLIIHTAALVDVFGKYTDNEIMKVNYYGTQTILAACVDLGIKYLIYTSSMEAIGPNKHGNPFIGHEHTLYDISPGHVYAKSKRMAEQLVMKANNSVIMNGAKLYTCCLRPTGIYGEGDKLTKVFYEQCKQHGNIMYRTVDDDAVHSRVYVGNVAWMHVLAAKYIQYPGSAIKGNAYFCYDYSPSCSYDMFNLLLMKPLGIEQGSRIPRWMLKMYACKNDMKRILFRKPSILNNYTLKISNTTFEVRTNNAELDFNYSPIFDVDVAFERTRKWLEESE

SEQ ID NO: 62 - protein, 346 aa, synthetic construct; identical to SEQ ID NO: 61

SEQ ID NO: 63 - protein, 346 aa, synthetic construct; identical to SEQ ID NO: 61

SEQ ID NO: 64 - protein, 346 aa, synthetic construct; identical to SEQ ID NO: 61

SEQ ID NO: 65 - protein, 346 aa, synthetic construct; identical to SEQ ID NO: 61

SEQ ID NO: 66 - protein, 267 aa, synthetic construct
MRYIIILAVLFINSIHAKITSYKFESVNFDSKIEWTGDGLYNISLKNYGIKTWQTMYTNVPEGTYDISGFPKNDFVSFWVKFEQGDYKVEEYCTGLCVEVKIGPPTVILTEYDDHINLFIEHPYATRGSKKIPIYKRGDMCDIYLLYTANFTFGDSEEPVTYDIDDYDCTSTGCSIDFATTEKVCVTAQGATEGFLEKITPWSSEVCLTPKKNVYTCAIRSKEDVPNFKDKIARVITRKFNKQSQSYLTKFLGSTSNDVTTFFSILD

SEQ ID NO: 67 - protein, 267 aa, synthetic construct
  VARIANT 32: note = Any amino acid
MRYIIILAVLFINSIHAKITSYKFESVNFDSXIEWTGDGLYNISLKNYGIKTWQTMYTNVPEGTYDISGFPKNDFVSFWVKFEQGDYKVEEYCTGLCVEVKIGPPTVILTEYDDHINLFIEHPYATRGSKKIPIYKRGDMCDIYLLYTANFTFGDSEEPVTYDIDDYDCTSTGCSIDFATTEKVCVTAQGATEGFLEKITPWSSEVCLTPKKNVYTCAIRSKEDVPNFKDKIARVITRKFNKQSQSYLTKFLGSTSNDVTTFFSILD

SEQ ID NO: 68 - protein, 267 aa, synthetic construct
  VARIANT 39: note = Any amino acid
MRYIIILAVLFINSIHAKITSYKFESVNFDSKIEWTGDXLYNISLKNYGIKTWQTMYTNVPEGTYDISGFPKNDFVSFWVKFEQGDYKVEEYCTGLCVEVKIGPPTVILTEYDDHINLFIEHPYATRGSKKIPIYKRGDMCDIYLLYTANFTFGDSEEPVTYDIDDYDCTSTGCSIDFATTEKVCVTAQGATEGFLEKITPWSSEVCLTPKKNVYTCAIRSKEDVPNFKDKIARVITRKFNKQSQSYLTKFLGSTSNDVTTFFSILD

SEQ ID NO: 69 - protein, 267 aa, synthetic construct
MRYIIILAVLFINSIHAKITSYKFESVNFDSKIEWTGDGLYNISLKNYGIKTWQTMYTNVPEGTYDISGFPKNDFVSFWVKFEQGDYKVEEYCTGLCVEVKIGPPTVRLTEYDDHINLFIEHPYATRGSKKIPIYKRGDMCDIYLLYTANFTFGDSEEPVTYDIDDYDCTSTGCSIDFATTEKVCVTAQGATEGFLEKITPWSSEVCLTPKKNVYTCAIRSKEDVPNFKDKIARVITRKFNKQSQSYLTKFLGSTSNDVTTFLSILD

SEQ ID NO: 70 - protein, 267 aa, synthetic construct; identical to SEQ ID NO: 66

SEQ ID NO: 71 - protein, 267 aa, synthetic construct; identical to SEQ ID NO: 68
  VARIANT 39: note = Any amino acid

SEQ ID NO: 72 - protein, 267 aa, synthetic construct; identical to SEQ ID NO: 69

SEQ ID NO: 73 - protein, 267 aa, synthetic construct; identical to SEQ ID NO: 69

SEQ ID NO: 74 - protein, 267 aa, synthetic construct; identical to SEQ ID NO: 69

SEQ ID NO: 75 - protein, 267 aa, synthetic construct; identical to SEQ ID NO: 69

SEQ ID NO: 76 - protein, 356 aa, synthetic construct
MMKMKMKMKMMVRIYFVSLSLLLFHSYAIDIENEITEFFNKMRDTLPAKDSKWLNPVCMFGGTMNDMAALGEPFSAKCPPIEDSLLSHRYKDYVVKWERLEKNRRRQVSNKRVKHGDLWIANYTSKFSNRRYLCTVTTKNGDCVQGVVRSHVWKPSSCIPKTYELGTYDKYGIDLYCGILYANHYNNITWYKDNKEINIDDFKYSQAGKELIIHNPELEDSGRYDCYVHYDDVRIKNDIVVSRCKILTVIPSQDHRFKLILDPKINVTIGEPANITCSAVSTSLFVDDVLIEWENPSGWIIGLDFGVYSILTSRGGITEATLYFENVTEEYIGNTYTCRGHNYYFDKTLTTTVVLE

SEQ ID NO: 77 - protein, 356 aa, synthetic construct; identical to SEQ ID NO: 76

SEQ ID NO: 78 - protein, 356 aa, synthetic construct; identical to SEQ ID NO: 76

SEQ ID NO: 79 - protein, 352 aa, synthetic construct
MMKMKMMVRIYFVSLSLLLFHSYAIDIENEITEFFNKMRDTLPAKDSKWLNPVCMFGGTMNDMAALGEPFSAKCPPIEDSLLSHRYKDYVVKWERLEKNRRRQVSNKRVKHGDLWIANYTSKFSNRRYLCTVTTKNGDCVQGVVRSHVWKPSSCIPKTYELGTYDKYGIDLYCGILYAKHYNNITWYKDNKEINIDDFKYSQAGKELIIHNPELEDSGRYDCYVHYDDVRIKNDIVVSRCKILTVIPSQDHRFKLILDPKINVTIGEPANITCSAVSTSLFVDDVLIEWENPSGWIIGLDFGVYSILTSRGGITEATLYFENVTEEYIGNTYTCRGHNYYFDKTLTTTVVLE

SEQ ID NO: 80 - protein, 356 aa, synthetic construct; identical to SEQ ID NO: 76

SEQ ID NO: 81 - protein, 356 aa, synthetic construct; identical to SEQ ID NO: 76

SEQ ID NO: 82 - protein, 351 aa, synthetic construct
MKMKMMVRIYFVSLSLLLFHSYAIDIENEITEFFNKMRDTLPAKDSKWLNPVCMFGGTMNDMAALGEPFSAKCPPIEDSLLSHRYKDYVVKWERLEKNRRRQVSNKRVKHGDLWIANYTSKFSNRRYLCTVTTKNGDCVQGVVRSHVWKPSSCIPKTYELGTYDKYGIDLYCGILYANHYNNITWYKDNKEINIDDFKYSQAGKELIIHNPELEDSGRYDCYVHYDDVRIKNDIVVSRCKILTVIPSQDHRFKLILDPKINVTIGEPANITCSAVSTSLFVDDVLIEWENPSGWIIGLDFGVYSILTSRGGITEATLYFENVTEEYIGNTYTCRGHNYYFDKTLTTTVVLE

SEQ ID NO: 83 - protein, 352 aa, synthetic construct; identical to SEQ ID NO: 79

SEQ ID NO: 84 - protein, 352 aa, synthetic construct; identical to SEQ ID NO: 79

SEQ ID NO: 85 - protein, 352 aa, synthetic construct; identical to SEQ ID NO: 79

SEQ ID NO: 86 - protein, 319 aa, synthetic construct
MEPILAPNPNRFVIFPIQYHDIWNMYKKAEASFWTVEEVDISKDINDWNKLTPDEKYFIKHVLAFFAASDGIVNENLAERFCTEVQITEARCFYGFQMAIENIHSEMYSLLIDTYVKDSNEKNYLFNAIETMPCVKKKADWAQKWIHDSAGYGERLIAFAAVEGIFFSGSFASIFWLKKRGLMPGLTFSNELISRDEGLHCDFACLMFKHLLHPPSEETVRFIITNAVSIEQEFLTVALPVKLIGMNCEMMKTYIEFVADRLISELGFKKIYNVTNPFDFMENISLEGKTNFFEKRVGEYQKMGVMSQEDNHFSLDVDF

SEQ ID NO: 87 - protein, 319 aa, synthetic construct
  VARIANT 28: note = Any amino acid
  VARIANT 31: note = Any amino acid
  VARIANT 38: note = Any amino acid
  VARIANT 137: note = Any amino acid
  VARIANT 168: note = Any amino acid
  VARIANT 179: note = Any amino acid
  VARIANT 271: note = Any amino acid
  VARIANT 293: note = Any amino acid
MEPILAPNPNRFVIFPIQYHDIWNMYKXAEXSFWTVEXVDISKDINDWNKLTPDEKYFIKHVLAFFAASDGIVNENLAERFCTEVQITEARCFYGFQMAIENIHSEMYSLLIDTYVKDSNEKNYLFNAIETMPCVKXKADWAQKWIHDSAGYGERLIAFAAVEGIFFXGSFASIFWLKXRGLMPGLTFSNELISRDEGLHCDFACLMFKHLLHPPSEETVRFIITNAVSIEQEFLTVALPVKLIGMNCEMMKTYIEFVADRLISELGFKKXYNVTNPFDFMENISLEGKTNFXEKRVGEYQKMGVMSQEDNHFSLDVDF

SEQ ID NO: 88 - protein, 319 aa, synthetic construct; identical to SEQ ID NO: 86

SEQ ID NO: 89 - protein, 319 aa, synthetic construct; identical to SEQ ID NO: 86

SEQ ID NO: 90 - protein, 319 aa, synthetic construct; identical to SEQ ID NO: 86

SEQ ID NO: 91 - protein, 189 aa, synthetic construct
MEPILAPNPNRFVIFPIQYHDIWNMYKKAEASFWTVEEVDISKDINDWNKLTPDEKYFIKHVLAFFAASDGIVNENLAERFCTEVQITEARCFYGFQMAIENIHSEMYSLLIDTYVKDSNEKNYLFNAIETMPCVKKKADWAQKWIHDSAGYGERLIAFAAVEGIFFSGSFASIFWLKKAWPNARTHVF

SEQ ID NO: 92 - protein, 158 aa, synthetic construct
MRSIAGLDKLKMEIFNVEELINMKPFKNMNKITINQKDNCILANRCFVKIDTPRYIPSTSISSSNIIRIRNHDFTLSELLYSPFHFQQPQFQYLLPGFVLTCIDKVSKQQKECKYCISNRGDDDSLSINIFIPTINKSIYIIIGLRMKNFWKPKFEIE

SEQ ID NO: 93 - protein, 158 aa, synthetic construct; identical to SEQ ID NO: 92

SEQ ID NO: 94 - protein, 158 aa, synthetic construct; identical to SEQ ID NO: 92

SEQ ID NO: 95 - protein, 158 aa, synthetic construct; identical to SEQ ID NO: 92

SEQ ID NO: 96 - protein, 158 aa, synthetic construct; identical to SEQ ID NO: 92

SEQ ID NO: 97 - protein, 161 aa, synthetic construct
MSSFVTNGYLPVTLEPHELTLDIKTNIRNAVYKAYLHREISGKMAKKIEIREDVELPLGEIVNNSVVINVPCVITYAYYHVGDIVRGTLNIEDESNVTIQCGDLICKLSRDSGTVSFSDSKYCFFRNGNAYDNGIEVSAVLMEAQQGTESSFVFLANIVDS

SEQ ID NO: 98 - protein, 161 aa, synthetic construct
  VARIANT 4: note = Any amino acid
  VARIANT 14: note = Any amino acid
  VARIANT 16: note = Any amino acid
  VARIANT 19: note = Any amino acid
  VARIANT 23: note = Any amino acid
  VARIANT 28: note = Any amino acid
  VARIANT 36: note = Any amino acid
  VARIANT 38: note = Any amino acid
  VARIANT 39: note = Any amino acid
  VARIANT 40: note = Any amino acid
  VARIANT 41: note = Any amino acid
  VARIANT 43: note = Any amino acid
  VARIANT 47: note = Any amino acid
  VARIANT 57: note = Any amino acid
  VARIANT 58: note = Any amino acid
  VARIANT 72: note = Any amino acid
  VARIANT 73: note = Any amino acid
  VARIANT 74: note = Any amino acid
  VARIANT 82: note = Any amino acid
  VARIANT 89: note = Any amino acid
  VARIANT 100: note = Any amino acid
  VARIANT 101: note = Any amino acid
  VARIANT 102: note = Any amino acid
  VARIANT 105: note = Any amino acid
  VARIANT 121: note = Any amino acid
  VARIANT 123: note = Any amino acid
  VARIANT 124: note = Any amino acid
  VARIANT 128: note = Any amino acid
  VARIANT 129: note = Any amino acid
  VARIANT 155: note = Any amino acid
  VARIANT 161: note = Any amino acid
MSSXVTNGYLPVTXEXHEXTLDXKTNIXNAVYKAYXHXXXXGXMAKXIEIREDVELXXGEIVNNSVVINVPXXXTYAYYHVXDIVRGTXNIEDESNVTIXXXDLXCKLSRDSGTVSFSDSXYXXFRNXXAYDNGIEVSAVLMEAQQGTESSFVFXANIVDX

SEQ ID NO: 99 - protein, 161 aa, synthetic construct; identical to SEQ ID NO: 97

SEQ ID NO: 100 - protein, 161 aa, synthetic construct; identical to SEQ ID NO: 97

SEQ ID NO: 101 - protein, 161 aa, synthetic construct; identical to SEQ ID NO: 97

SEQ ID NO: 102 - protein, 153 aa, synthetic construct
MEKREVNKALYDLQRSAMVYSSNDTPPRWSTTMDADTRPTDSDADAIIDDVSREKSMREDHKSFDDVIPVKKIIYWKGVNPVTVINEYCQITRRDWSFRIESVGPSNSPTFYACVDIDGRVFDKADGKSKRDAKNNAAKLAVDKLLSYVIIRF

SEQ ID NO: 103 - protein, 153 aa, synthetic construct; identical to SEQ ID NO: 102

SEQ ID NO: 104 - protein, 153 aa, synthetic construct; identical to SEQ ID NO: 102

SEQ ID NO: 105 - protein, 153 aa, synthetic construct
MEKREVNKALYDLQRSTMVYSSDDTPPRWSTTMDADTRPTDSDADAIIDDVSREKSMREDNKSFDDVIPVKKIIYWKGVNPVTVINEYCQITRRDWSFRIESVGPSNSPTFYACVDIDGRVFDKADGKSKRDAKNNAAKLAVDKLLSYVIIRF

SEQ ID NO: 106 - protein, 153 aa, synthetic construct; identical to SEQ ID NO: 102

SEQ ID NO: 107 - protein, 153 aa, synthetic construct; identical to SEQ ID NO: 102

SEQ ID NO: 108 - protein, 153 aa, synthetic construct; identical to SEQ ID NO: 102

SEQ ID NO: 109 - protein, 153 aa, synthetic construct; identical to SEQ ID NO: 105

SEQ ID NO: 110 - protein, 153 aa, synthetic construct; identical to SEQ ID NO: 105

SEQ ID NO: 111 - protein, 153 aa, synthetic construct; identical to SEQ ID NO: 105

SEQ ID NO: 112 - protein, 259 aa, synthetic construct
MENVYISSYSSNEQISMAVVTTDIRELLSQYVDDANLENLIEWAMEKSSKYYIKNIGNTKSNIEETKFESKNNIGIEYSKDSKNKLSYRNKPSIATHLEYKTLCDMIKGTSGTEKEFLRYLLFGIKCIKKGVEYNIDKIKDVSYNDYFNVLDEKYNTPCPNCKSRNTTPMMIQTRAADEPPLVRHACRDCKQHFKPPKFRAFRNLNVTTQSIHENKEITEILPDNNPSPPESPEPASPIDDGLIRATFDRNDEPPEDDE

SEQ ID NO: 113 - protein, 259 aa, synthetic construct; identical to SEQ ID NO: 112

SEQ ID NO: 114 - protein, 259 aa, synthetic construct; identical to SEQ ID NO: 112

SEQ ID NO: 115 - protein, 259 aa, synthetic construct
MENVYISSYSSNEQISMAVVTTDIRELLSQYVDDANLENLIEWAMEKSSKYYIKNIGNTKSNIEETKFESKNNIGIEYSKDSRNKLSYRNKPSIATNLEYKTLCDMIKGTSGTEKEFLRYLLFGIKCIKKGVEYNIDKIKDVSYNDYFNVLDEKYNTPCPNCKSRNTTPMMIQTRAADEPPLVRHACRDCKQHFKPPKFRAFRNLNVTTQSIHENKEITEILPDNNPSPPESPEPASPIDDGLIRATFDRNDEPPEDDE

SEQ ID NO: 116 - protein, 259 aa, synthetic construct; identical to SEQ ID NO: 112

SEQ ID NO: 117 - protein, 63 aa, synthetic construct
MVFQLVCSTCGKDISHERYKLIIRKKSLKDVLVSVKNECCRLKLSTQIEPQRNLTVQPLLDIN

SEQ ID NO: 118 - protein, 63 aa, synthetic construct
  VARIANT 60: note = Any amino acid
MVFQLVCSTCGKDISHERYKLIIRKKSLKDVLVSVKNECCRLKLSTQIEPQRNLTVQPLXDIN

SEQ ID NO: 119 - protein, 63 aa, synthetic construct; identical to SEQ ID NO: 117

SEQ ID NO: 120 - protein, 63 aa, synthetic construct; identical to SEQ ID NO: 117

SEQ ID NO: 121 - protein, 63 aa, synthetic construct; identical to SEQ ID NO: 117

SEQ ID NO: 122 - protein, 210 aa, synthetic construct
MAWSITNKADTSSFTKMAEIRAHLRNSAENKDKNEDIFPEDVIIPSTKPKTKRTTTPRKPAATKRSTKKDKEKEEVEEVVIEEYHQTTEENSPPPSSSPGVGDIVESVAAVELDDSDGDDEPMVQVEAGKVNHSARSDLSDLKVATDNIVKDLKKIITRISAVSTVLEDVQAAGISRQFTSMTKAITTLSDLVTEGKSKVVRKKVKTCKK

SEQ ID NO: 123 - protein, 210 aa, synthetic construct; identical to SEQ ID NO: 122

SEQ ID NO: 124 - protein, 210 aa, synthetic construct; identical to SEQ ID NO: 122

SEQ ID NO: 125 - protein, 213 aa, synthetic construct
MAWSITNKADTSSFTKMAEIRAHLRNSAENKDKNEDIFPEDVIIPSTKPKTKRTTTPRKPAATKRSTKKDKEKEEVEEVEEVVIEEYHQTTEENSPPPSSSPGVGDIVESVAAVELDDSDGDDEPMVQVEAGKVNHSARSDLSDLKVATDNIVKDLKKIITRISAVSTVLEDVQAAGISRQFTSMTKAITTLSDLVTEGKSKVVRKKVKTCKK

SEQ ID NO: 126 - protein, 210 aa, synthetic construct; identical to SEQ ID NO: 122

SEQ ID NO: 127 - protein, 269 aa, synthetic construct
MSKVIKKRVETSPRPTASSDSLQTCAGVIEYAKSISKSNAKCIECVTLNASQYANCSSISIKLTDSLSSQMTSTFIMLEGETKLYKNKSKQDRSDGYFLKIKVTAASPMLYQLLEAVYGNITHKERIPNSLHSLSAETITEKTFKDESIFINKLNGAMVEYVSTGESSILRSIEGELESLSKRERQLAKAIITPVVFYRSGTETKITFALKKLIIDREVVANVIGLSGDSERVSMTENVEEDLVRNLGLVDIDDEYDEDSDKEKPIFNV

SEQ ID NO: 128 - protein, 269 aa, synthetic construct
  VARIANT 41: note = Any amino acid
  VARIANT 99: note = Any amino acid
MSKVIKKRVETSPRPTASSDSLQTCAGVIEYAKSISKSNAXCIECVTLNASQYANCSSISIKLTDSLSSQMTSTFIMLEGETKLYKNKSKQDRSDGYFXKIKVTAASPMLYQLLEAVYGNITHKERIPNSLHSLSAETITEKTFKDESIFINKLNGAMVEYVSTGESSILRSIEGELESLSKRERQLAKAIITPVVFYRSGTETKITFALKKLIIDREVVANVIGLSGDSERVSMTENVEEDLVRNLGLVDIDDEYDEDSDKEKPIFNV

SEQ ID NO: 129 - protein, 269 aa, synthetic construct; identical to SEQ ID NO: 127

SEQ ID NO: 130 - protein, 269 aa, synthetic construct
MSKVIKKRVETSPRPTASSDSLQTCAGVIEYAKSISKSNAKCIEYVTLNASQYANCSSISIKLTDSLSSQMTSTFIMLEGETKLYKNKSKQDRSDGYFLKIKVTAASPMLYQLLEAVYGNITHKERIPNSLHSLSAETITEKTFKDESIFINKLNGAMVEYVSTGESSILRSIEGELESLSKRERQLAKAIITPVVFYRSGTETKITFALKKLIIDREVVANVIGLSGDSERVSMTENVEEDLVRNLGLVDIDDEYDEDSDKEKPIFNV

SEQ ID NO: 131 - protein, 269 aa, synthetic construct; identical to SEQ ID NO: 127

SEQ ID NO: 132 - protein, 220 aa, synthetic construct
MVYKLVLLFCIASLGYSVEYKNTICPHRQDYRYWYFVAELTIGVNYDINSTIIGECHMSESYIDRNANIVLTGYGLKVNMTIMDTDQRFVAAAEGVGKDNKLSVLLFTTQRLDKVHHNISVTITCMEMNCGTTKYNSDLPESIHKSSSCDITINGSCVTCVNLETDPTKINPHYLHPKNKYLYHNSEYSMRGSYGVTFIDELNQCLLDIKELSYDICYRE

SEQ ID NO: 133 - protein, 220 aa, synthetic construct; identical to SEQ ID NO: 132

SEQ ID NO: 134 - protein, 220 aa, synthetic construct; identical to SEQ ID NO: 132

SEQ ID NO: 135 - protein, 220 aa, synthetic construct
MVYKLVLLFCIASLGYSVEYKNTICPPRQDYRYWYFVAELTIGVNYDINSTIIGECHMSESYIDRNANIVLTGYGLKINMTIMDTDQRFVAAAEGVGKDNKLSVLLFTTQRLDKVHHNISVTITCMEMNCGTTKYNSDLPESIHKSSSCDITINGSCVTCVNLETDPTKINPHYLHPKNKYLYHNSEYSMRGSYGVTFIDELNQCLLDIKELSYDICYRE

SEQ ID NO: 136 - protein, 220 aa, synthetic construct; identical to SEQ ID NO: 132

SEQ ID NO: 137 - protein, 665 aa, synthetic construct
MFMYPEFARKALSKLISKKLNIEKVSSKHQLVLLDYGLHGLLPKSLYLEAINSDILNVRFFPPEIINVTDIVKALQNSCRVDEYLKAVSLYHKNSLMVSGPNVVKLMIEYNLLTHSDLEWLINENVVKATYLLKINAYMINFKIDLTVDEIIDLVKDIPVGATLHLYNILNNIDLDIILRISDEYNIPPVHDILSKLTNEEMCIKLVTKYPMDNVINFINQDVRYSPTFIKTIKDFVNEHLPTMYDGLNDYLHSVIIDEDLIEEYKIKSVAMFNLEYKTDIDTLTLDEQIFVEVNISYYDFRYRQFADEFRDYIMIKERRQITMQSGDRIRRFRRPMSLRSTIIKKDTDSLEDILSHIDNAKKNSKVSIEDVERIISSFRLNPCVVRRTMLSDIDIKTKIMVLKIAKDWKSCTLTLSVKGIMVTDTINTVLSKILHHHRNIFKYLTSVENKEIAVCNCSRCLSLFYRKLKSVRCDLRTDDGLLDRLYDLTRYALHGKINQNLIGQRCWGPLTEILFNENKKKKLNNLMEYIKISDMLVYGHYIEKTLIPITDSLSFKLFVDTMSVLNDQYAKVVIFFNTIIEYIIATIYYRLTVLDNYTNVKHFVSKVLHTVMEACGVLFSHIKVNDKIEYELEEMVDKGTVPSYLYHLSINVISIILDDINGTR

SEQ ID NO: 138 - protein, 665 aa, synthetic construct
  VARIANT 364: note = Any amino acid
  VARIANT 408: note = Any amino acid
  VARIANT 523: note = Any amino acid
MFMYPEFARKALSKLISKKLNIEKVSSKHQLVLLDYGLHGLLPKSLYLEAINSDILNVRFFPPEIINVTDIVKALQNSCRVDEYLKAVSLYHKNSLMVSGPNVVKLMIEYNLLTHSDLEWLINENVVKATYLLKINAYMINFKIDLTVDEIIDLVKDIPVGATLHLYNILNNIDLDIILRISDEYNIPPVHDILSKLTNEEMCIKLVTKYPMDNVINFINQDVRYSPTFIKTIKDFVNEHLPTMYDGLNDYLHSVIIDEDLIEEYKIKSVAMFNLEYKTDIDTLTLDEQIFVEVNISYYDFRYRQFADEFRDYIMIKERRQITMQSGDRIRRFRRPMSLRSTIIKKDTDSLEDILSHIDNAKKXSKVSIEDVERIISSFRLNPCVVRRTMLSDIDIKTKIMVLKIAKXWKSCTLTLSVKGIMVTDTINTVLSKILHHHRNIFKYLTSVENKEIAVCNCSRCLSLFYRKLKSVRCDLRTDDGLLDRLYDLTRYALHGKINQNLIGQRCWGPLTEILFNENKKKXLNNLMEYIKISDMLVYGHYIEKTLIPITDSLSFKLFVDTMSVLNDQYAKVVIFFNTIIEYIIATIYYRLTVLDNYTNVKHFVSKVLHTVMEACGVLFSHIKVNDKIEYELEEMVDKGTVPSYLYHLSINVISIILDDINGTR

SEQ ID NO: 139 - protein, 665 aa, synthetic construct
  VARIANT 351: note = Any amino acid
  VARIANT 357: note = Any amino acid
  VARIANT 377: note = Any amino acid
  VARIANT 384: note = Any amino acid
  VARIANT 388: note = Any amino acid
  VARIANT 392: note = Any amino acid
  VARIANT 400: note = Any amino acid
  VARIANT 422: note = Any amino acid
  VARIANT 454: note = Any amino acid
MFMYPEFARKALSKLISKKLNIEKVSSKHQLVLLDYGLHGLLPKSLYLEAINSDILNVRFFPPEIINVTDIVKALQNSCRVDEYLKAVSLYHKNSLMVSGPNVVKLMIEYNLLTHSDLEWLINENVVKATYLLKINAYMINFKIDLTVDEIIDLVKDIPVGATLHLYNILNNIDLDIILRISDEYNIPPVHDILSKLTNEEMCIKLVTKYPMDNVINFINQDVRYSPTFIKTIKDFVNEHLPTMYDGLNDYLHSVIIDEDLIEEYKIKSVAMFNLEYKTDIDTLTLDEQIFVEVNISYYDFRYRQFADEFRDYIMIKERRQITMQSGDRIRRFRRPMSLRSTIIKKDTDSXEDILSXIDNAKKNSKVSIEDVERIIXSFRLNPXVVRXTMLXDIDIKTKXMVLKIAKDWKSCTLTLSVKGIXVTDTINTVLSKILHHHRNIFKYLTSVENKEIXVCNCSRCLSLFYRKLKSVRCDLRTDDGLLDRLYDLTRYALHGKINQNLIGQRCWGPLTEILFNENKKKKLNNLMEYIKISDMLVYGHYIEKTLIPITDSLSFKLFVDTMSVLNDQYAKVVIFFNTIIEYIIATIYYRLTVLDNYTNVKHFVSKVLHTVMEACGVLFSHIKVNDKIEYELEEMVDKGTVPSYLYHLSINVISIILDDINGTR

SEQ ID NO: 140 - protein, 665 aa, synthetic construct
MFMYPEFARKALSKLISKKLNIEKVSSKHQLVLLDYGLHGLLPKSLYLEAINSDILNVRFFPPEIINVTDIVKALQNSCRVDEYLKAVSLYHKNSLMVSGPNVVKLMIEYNLLTHSDLEWLINENVVKATYLLKINAYMINFKIDLTVDEIIDLVKDIPVGATLHLYNILNNIDLDIILRISDEYNIPPVHDILSKLTDEEMCIKLVTKYPMDNVINFINQDVRYSPTFIKTIKDFVNEHLPTMYDGLNDYLHSVIIDEDLIEEYKIKSVAMFNLEYKTDIDTLTLDEQIFVEVNISYYDFRYRQFADEFRDYIMIKERRQITMQSGDRIRRFRRPMSLRSTIIKKDTDSLEDILSHIDNAKKNSKVSIEDVERIISSFRLNPCVVRRTMLSDIDIKTKIMVLKIAKDWKSCTLTLSVKGIMVTDTINTVLSKILHHHRNVFKYLTSVENKEIAVCNCSRCLSLFYRKLKSVRCDLRTDDGLLDRLYDLTRYALHGKINQNLIGQRCWGPLTEILFNENKKKKLNNLMEYIKISDMLVYGHYIEKTLIPITDSLSFKLFVDTMSVLNDQYAKVVIFFNTIIEYIIATIYYRLTVLDNYTNVKHFVSKVLHTVMEACGVLFSHIKVNDKIEYELEEMVDKGTVPSYLYHLSINVISIILDDINGTR

SEQ ID NO: 141 - protein, 665 aa, synthetic construct; identical to SEQ ID NO: 139
  VARIANT 351: note = Any amino acid
  VARIANT 357: note = Any amino acid
  VARIANT 377: note = Any amino acid
  VARIANT 384: note = Any amino acid
  VARIANT 388: note = Any amino acid
  VARIANT 392: note = Any amino acid
  VARIANT 400: note = Any amino acid
  VARIANT 422: note = Any amino acid
  VARIANT 454: note = Any amino acid

SEQ ID NO: 142 - protein, 19 aa, synthetic construct
MDWIWRILFLVGAATGAHS

SEQ ID NO: 143 - protein, 27 aa, synthetic construct
MGGAAARLGAVILFVVIVGLHGVRSKY

SEQ ID NO: 144 - protein, 25 aa, synthetic construct
MGRLTSGVGTAALLVVAVGLRVVCA

SEQ ID NO: 145 - protein, 28 aa, synthetic construct
MGRLTSGVGTAALLVVAVGLRVVCAKYA

SEQ ID NO: 146 - protein, 19 aa, synthetic construct
MFVFLVLLPLVSSQCVNLT

SEQ ID NO: 147 - protein, 21 aa, synthetic construct
MDWIWRILFLVGAATGAHSQM

SEQ ID NO: 148 - protein, 19 aa, synthetic construct
MDWTWRVFCLLAVAPGAHS

SEQ ID NO: 149 - protein, 20 aa, synthetic construct
METPAQLLFLLLLWLPDTTG

SEQ ID NO: 150 - protein, 18 aa, synthetic construct
MDWTWILFLVAAATRVHS

SEQ ID NO: 151 - protein, 24 aa, synthetic construct
MLGSNSGQRVVFTILLLLVAPAYS

SEQ ID NO: 152 - protein, 17 aa, synthetic construct
MKCLLYLAFLFIGVNCA

SEQ ID NO: 153 - protein, 15 aa, synthetic construct
MWLVSLAIVTACAGA

SEQ ID NO: 154 - protein, 15 aa, synthetic construct
MFVFLVLLPLVSSQC

SEQ ID NO: 155 - DNA, 53 bp, synthetic construct
  source 1..53: mol_type = other DNA
agaataaactagtattcttctggtccccacagactcagagagaacccgccacc

SEQ ID NO: 156 - skipped

SEQ ID NO: 157 - protein, 26 aa, synthetic construct
MRVMAPRTLILLLSGALALTETWAGS

SEQ ID NO: 158 - protein, 250 aa, synthetic construct; identical to SEQ ID NO: 31

SEQ ID NO: 159 - DNA, 753 bp, synthetic construct
  source 1..753: mol_type = other DNA
atgggtgccgcagcaagcatacagacgactgtgaatacactcagtgaacgtatctcgtctaaattagaacaagaagcgaacgctagtgctcaaacaaaatgtgatatagaaatcggaaatttttatatccgacaaaaccatggatgtaacatcactgttaaaaatatgtgctctgcggacgcggatgctcagttggatgctgtgttatcagccgctacagaaacatatagtggattaacaccggaacaaaaagcatacgtaccagctatgtttactgctgcgttaaacattcagacgagtgtaaacactgttgttagagattttgaaaattatgtgaaacagacttgtaattctagcgctgttgtcgataacaaattaaagatacaaaacgtaattatagatgaatgttacggagccccaggatctccaacaaatttggaatttattaatacaggatctagcaaaggaaattgtgccattaaggcgttgatgcaattgactactaaggccactactcaaatagcacctagacaagttgctggtacaggagttcagttttatatgattgttatcggtgttataatattggcagcgttgtttatgtactatgccaagcgtatgctgttcacatccaccaatgataaaatcaaacttattttagccaataaggaaaacgtccattggactacttacatggacacattctttagaacttctccgatgattattgctaccacggatatacaaaactga

SEQ ID NO: 160 - protein, 25 aa, synthetic construct
MGGAAARLGAVILFVVIVGLHGVRG

SEQ ID NO: 161 - DNA, 75 bp, synthetic construct
  source 1..75: mol_type = other DNA
atggggggggctgccgccaggttgggggccgtgattttgtttgtcgtcatagtgggcctccatggggtccgcggc

SEQ ID NO: 162 - protein, 275 aa, synthetic construct
MGGAAARLGAVILFVVIVGLHGVRGMGAAASIQTTVNTLSERISSKLEQEANASAQTKCDIEIGNFYIRQNHGCNITVKNMCSADADAQLDAVLSAATETYSGLTPEQKAYVPAMFTAALNIQTSVNTVVRDFENYVKQTCNSSAVVDNKLKIQNVIIDECYGAPGSPTNLEFINTGSSKGNCAIKALMQLTTKATTQIAPRQVAGTGVQFYMIVIGVIILAALFMYYAKRMLFTSTNDKIKLILANKENVHWTTYMDTFFRTSPMIIATTDIQN

SEQ ID NO: 163 - DNA, 828 bp, synthetic construct
  source 1..828: mol_type = other DNA
atggggggggctgccgccaggttgggggccgtgattttgtttgtcgtcatagtgggcctccatggggtccgcggcatgggtgccgcagcaagcatacagacgactgtgaatacactcagtgaacgtatctcgtctaaattagaacaagaagcgaacgctagtgctcaaacaaaatgtgatatagaaatcggaaatttttatatccgacaaaaccatggatgtaacatcactgttaaaaatatgtgctctgcggacgcggatgctcagttggatgctgtgttatcagccgctacagaaacatatagtggattaacaccggaacaaaaagcatacgtaccagctatgtttactgctgcgttaaacattcagacgagtgtaaacactgttgttagagattttgaaaattatgtgaaacagacttgtaattctagcgctgttgtcgataacaaattaaagatacaaaacgtaattatagatgaatgttacggagccccaggatctccaacaaatttggaatttattaatacaggatctagcaaaggaaattgtgccattaaggcgttgatgcaattgactactaaggccactactcaaatagcacctagacaagttgctggtacaggagttcagttttatatgattgttatcggtgttataatattggcagcgttgtttatgtactatgccaagcgtatgctgttcacatccaccaatgataaaatcaaacttattttagccaataaggaaaacgtccattggactacttacatggacacattctttagaacttctccgatgattattgctaccacggatatacaaaactga

SEQ ID NO: 164 - protein, 110 aa, synthetic construct; identical to SEQ ID NO: 1

SEQ ID NO: 165 - DNA, 333 bp, synthetic construct
  source 1..333: mol_type = other DNA
atggacggaactcttttccccggagatgacgatcttgcaattccagcaactgaatttttctctacaaaggctgctaaaaatccagagactaaacgcgaagcaattgttaaagcctatggagacgataatgaggaaactctcaaacaacggctaactaatttggaaaaaaagattactaatataacaacaaagtttgaacaaatagaaaagtgttgtaaacgcaacgatgaagttctatttaggttggaaaatcacgctgaaactctaagagcggctatgatatctctggctaaaaagattgatgtacagactggacggcatccatatgagtaa

SEQ ID NO: 166 - protein, 135 aa, synthetic construct
MGGAAARLGAVILFVVIVGLHGVRGMDGTLFPGDDDLAIPATEFFSTKAAKNPETKREAIVKAYGDDNEETLKQRLTNLEKKITNITTKFEQIEKCCKRNDEVLFRLENHAETLRAAMISLAKKIDVQTGRHPYE

SEQ ID NO: 167 - DNA, 408 bp, synthetic construct
  source 1..408: mol_type = other DNA
atggggggggctgccgccaggttgggggccgtgattttgtttgtcgtcatagtgggcctccatggggtccgcggcatggacggaactcttttccccggagatgacgatcttgcaattccagcaactgaatttttctctacaaaggctgctaaaaatccagagactaaacgcgaagcaattgttaaagcctatggagacgataatgaggaaactctcaaacaacggctaactaatttggaaaaaaagattactaatataacaacaaagtttgaacaaatagaaaagtgttgtaaacgcaacgatgaagttctatttaggttggaaaatcacgctgaaactctaagagcggctatgatatctctggctaaaaagattgatgtacagactggacggcatccatatgagtaa

SEQ ID NO: 168 - protein, 110 aa, synthetic construct
MDGTLFPGDDDLAIPATEFFSTKAAKNPETKREAIVKAYGDDNEETLKQRLTNLEKKITNITTKFEQIEKAAKRNDEVLFRLENHAETLRAAMISLAKKIDVQTGRHPYE

SEQ ID NO: 169 - DNA, 333 bp, synthetic construct
  source 1..333: mol_type = other DNA
atggacggaactcttttccccggagatgacgatcttgcaattccagcaactgaatttttctctacaaaggctgctaaaaatccagagactaaacgcgaagcaattgttaaagcctatggagacgataatgaggaaactctcaaacaacggctaactaatttggaaaaaaagattactaatataacaacaaagtttgaacaaatagaaaaggctgctaaacgcaacgatgaagttctatttaggttggaaaatcacgctgaaactctaagagcggctatgatatctctggctaaaaagattgatgtacagactggacggcatccatatgagtaa

SEQ ID NO: 170 - protein, 135 aa, synthetic construct
MGGAAARLGAVILFVVIVGLHGVRGMDGTLFPGDDDLAIPATEFFSTKAAKNPETKREAIVKAYGDDNEETLKQRLTNLEKKITNITTKFEQIEKAAKRNDEVLFRLENHAETLRAAMISLAKKIDVQTGRHPYE

SEQ ID NO: 171 - DNA, 408 bp, synthetic construct
  source 1..408: mol_type = other DNA
atggggggggctgccgccaggttgggggccgtgattttgtttgtcgtcatagtgggcctccatggggtccgcggcatggacggaactcttttccccggagatgacgatcttgcaattccagcaactgaatttttctctacaaaggctgctaaaaatccagagactaaacgcgaagcaattgttaaagcctatggagacgataatgaggaaactctcaaacaacggctaactaatttggaaaaaaagattactaatataacaacaaagtttgaacaaatagaaaaggctgctaaacgcaacgatgaagttctatttaggttggaaaatcacgctgaaactctaagagcggctatgatatctctggctaaaaagattgatgtacagactggacggcatccatatgagtaa

SEQ ID NO: 172 - protein, 181 aa, synthetic construct; identical to SEQ ID NO: 11

SEQ ID NO: 173 - DNA, 546 bp, synthetic construct
  source 1..546: mol_type = other DNA
atgatgacaccagaaaacgacgaagagcagacatctgtgttctccgctactgtttacggagacaaaattcagggaaagaataaacgcaaacgcgtgattggtctatgtattagaatatctatggttatttcactactatctatgattaccatgtccgcgtttctcatagtgcgcctaaatcaatgcatgtctgctaacaaggctgctattactgactccgctgttgccgttgctgcggcatcatctactcatagaaaggttgtgtctagcactacacaatatgatcacaaagaaagctgtaatggtttatattaccagggttcttgttatatattacattcagactataagtcattcgaggatgctaaagcaaactgcgctgcggaatcatcaacactacccaataaatccgatgtcttgactacctggctcattgattatgttgaggatacatggggatctgatggtaatccaattacaaaaactacatccgattatcaagattctgatgtatcacaagaagttagaaagtatttttgtacataa

SEQ ID NO: 174 - protein, 93 aa, synthetic construct
SSTTQYDHKESCNGLYYQGSCYILHSDYKSFEDAKANCAAESSTLPNKSDVLTTWLIDYVEDTWGSDGNPITKTTSDYQDSDVSQEVRKYFCT

SEQ ID NO: 175 - DNA, 279 bp, synthetic construct
  source 1..279: mol_type = other DNA
tctagcactacacaatatgatcacaaagaaagctgtaatggtttatattaccagggttcttgttatatattacattcagactataagtcattcgaggatgctaaagcaaactgcgctgcggaatcatcaacactacccaataaatccgatgtcttgactacctggctcattgattatgttgaggatacatggggatctgatggtaatccaattacaaaaactacatccgattatcaagattctgatgtatcacaagaagttagaaagtatttttgtaca

SEQ ID NO: 176 - protein, 10 aa, synthetic construct
GGSGGGGSGG

SEQ ID NO: 177 - DNA, 30 bp, synthetic construct
  source 1..30: mol_type = other DNA
ggcggctctggaggaggcggctccggaggc

SEQ ID NO: 178 - protein, 221 aa, synthetic construct
MGGAAARLGAVILFVVIVGLHGVRGSSTTQYDHKESCNGLYYQGSCYILHSDYKSFEDAKANCAAESSTLPNKSDVLTTWLIDYVEDTWGSDGNPITKTTSDYQDSDVSQEVRKYFCTGGSGGGGSGGSSTTQYDHKESCNGLYYQGSCYILHSDYKSFEDAKANCAAESSTLPNKSDVLTTWLIDYVEDTWGSDGNPITKTTSDYQDSDVSQEVRKYFCT

SEQ ID NO: 179 - DNA, 666 bp, synthetic construct
  source 1..666: mol_type = other DNA
atggggggggctgccgccaggttgggggccgtgattttgtttgtcgtcatagtgggcctccatggggtccgcggctctagcactacacaatatgatcacaaagaaagctgtaatggtttatattaccagggttcttgttatatattacattcagactataagtcattcgaggatgctaaagcaaactgcgctgcggaatcatcaacactacccaataaatccgatgtcttgactacctggctcattgattatgttgaggatacatggggatctgatggtaatccaattacaaaaactacatccgattatcaagattctgatgtatcacaagaagttagaaagtatttttgtacaggcggctctggaggaggcggctccggaggctctagcactacacaatatgatcacaaagaaagctgtaatggtttatattaccagggttcttgttatatattacattcagactataagtcattcgaggatgctaaagcaaactgcgctgcggaatcatcaacactacccaataaatccgatgtcttgactacctggctcattgattatgttgaggatacatggggatctgatggtaatccaattacaaaaactacatccgattatcaagattctgatgtatcacaagaagttagaaagtatttttgtacataa

SEQ ID NO: 180 - protein, 317 aa, synthetic construct; identical to SEQ ID NO: 21

SEQ ID NO: 181 - DNA, 954 bp, synthetic construct
  source 1..954: mol_type = other DNA
atgaaaacgatttccgttgttacgttgttatgcgtactacctgctgttgtttattcaacatgtactgtacccactatgaataacgctaaattaacgtctaccgaaacatcgtttaatgataaacagaaagttacgtttacatgtgattcaggatatcattctttggatccaaatgctgtctgtgaaacagataaatggaaatacgaaaatccatgcaagaaaatgtgcacagtttctgattatgtctctgaactatatgataagccattatacgaagtgaattccaccatgacactaagttgcaacggtgaaacaaaatattttcgttgtgaagaaaaaaatggaaatacttcttggaatgatactgtcacgtgtcctaatgcggaatgtcaacctcttcaattagaacacggatcgtgtcaaccagttaaagaaaaatactcatttggggaatatatgactatcaactgtgatgttggatatgaggttattggtgtttcgtatataagttgtacggctaattcttggaatgttattccatcatgtcaacaaaaatgtgatataccgtccctatctaatggattaatttccggatctacattttctatcggtggcgttatacatcttagttgtaaaagtggttttacactaacggggtctccatcatccacatgtatcgacggtaaatggaatcccatactcccaacatgtgtacgatctaacgaagaatttgatccagtggatgatggtcccgacgatgagacagatctgagcaaactctcgaaagacgttgtacaatatgaacaagaaatagaatcgttagaagcaacttatcatataatcataatggcgttgacaattatgggtgtcatatttctaatctccattatagtattagtttgttcctgtgacaaaaataatgaccaatataagttccataaattgctaccgtga

SEQ ID NO: 182 - protein, 317 aa, synthetic construct
MKTISVVTLLCVLPAVVYSTCTVPTMNNAKLTSTETSFNDKQKVTFTCDSGYHSLDPNAVCETDKWKYENPCKKMCTVSDYVSELYDKPLYEVNSTMTLSCNGETKYFRCEEKNGNTSWNDTVTCPNAECQPLQLEHGSAQPVKEKYSFGEYMTINCDVGYEVIGVSYISCTANSWNVIPSCQQKCDIPSLSNGLISGSTFSIGGVIHLSCKSGFTLTGSPSSTCIDGKWNPILPTCVRSNEEFDPVDDGPDDETDLSKLSKDVVQYEQEIESLEATYHIIIMALTIMGVIFLISIIVLVCSCDKNNDQYKFHKLLP

SEQ ID NO: 183 - DNA, 954 bp, synthetic construct
  source 1..954: mol_type = other DNA
atgaaaacgatttccgttgttacgttgttatgcgtactacctgctgttgtttattcaacatgtactgtacccactatgaataacgctaaattaacgtctaccgaaacatcgtttaatgataaacagaaagttacgtttacatgtgattcaggatatcattctttggatccaaatgctgtctgtgaaacagataaatggaaatacgaaaatccatgcaagaaaatgtgcacagtttctgattatgtctctgaactatatgataagccattatacgaagtgaattccaccatgacactaagttgcaacggtgaaacaaaatattttcgttgtgaagaaaaaaatggaaatacttcttggaatgatactgtcacgtgtcctaatgcggaatgtcaacctcttcaattagaacacggatcggctcaaccagttaaagaaaaatactcatttggggaatatatgactatcaactgtgatgttggatatgaggttattggtgtttcgtatataagttgtacggctaattcttggaatgttattccatcatgtcaacaaaaatgtgatataccgtccctatctaatggattaatttccggatctacattttctatcggtggcgttatacatcttagttgtaaaagtggttttacactaacggggtctccatcatccacatgtatcgacggtaaatggaatcccatactcccaacatgtgtacgatctaacgaagaatttgatccagtggatgatggtcccgacgatgagacagatctgagcaaactctcgaaagacgttgtacaatatgaacaagaaatagaatcgttagaagcaacttatcatataatcataatggcgttgacaattatgggtgtcatatttctaatctccattatagtattagtttgttcctgtgacaaaaataatgaccaatataagttccataaattgctaccgtga

SEQ ID NO: 184 - protein, 324 aa, synthetic construct; identical to SEQ ID NO: 51

SEQ ID NO: 185 - DNA, 975 bp, synthetic construct
  source 1..975: mol_type = other DNA
atggcggcggtgaaaactcctgttattgttgtgccagttattgatagacctccatcagaaacatttcctaatgttcatgagcatattaatgatcagaagttcgatgatgtgaaggacaatgaagttatgcaagaaaaaagagatgttgtgatagtcaatgatgatccagatcattacaaggattatgtgtttatacaatggactggaggaaatattagagatgatgacaagtatactcacttcttttcagggttttgtaacactatgtgtacagaggaaacgaaaagaaatatcgctagacatttagccctatgggattctaaattttttaccgagctagaaaataaaaatgtagaatatgtagttattatagaaaacgataacgttattgaggatattacgtttcttcgtcccgtcttgaaggcaatacatgacaagaaaatagatatcctacagatgagagaaattattacaggaaataaagttaaaaccgagcttgtaatagacaaagatcatgccatatttacatatacaggagggtatgatgttagcttatccgcctatattattagagttactacggcgctgaacatcgtagatgaaattataaagtctggaggtctatcatcgggattttattttgaaatagccagaatcgaaaacgaaatgaagattaacaggcaaataatggataactctgccaaatatgtagaacacgatcctcgtcttgttgcagaacaccgcttcgaaaccatgaaaccaaatttttggtctagaataggaacggtagctgctaaacgttatccaggagttatgtacacgtttactactccattgatttcattttttggattgtttgatattaatgttataggtttgattgtaattttgtttattatgtttatgctcatctttaatgttaaatctaaactgttatggttccttacaggaacatttgttaccgcatttatctaa

SEQ ID NO: 186 - protein, 349 aa, synthetic construct
MGGAAARLGAVILFVVIVGLHGVRGMAAVKTPVIVVPVIDRPPSETFPNVHEHINDQKFDDVKDNEVMQEKRDVVIVNDDPDHYKDYVFIQWTGGNIRDDDKYTHFFSGFCNTMCTEETKRNIARHLALWDSKFFTELENKNVEYVVIIENDNVIEDITFLRPVLKAIHDKKIDILQMREIITGNKVKTELVIDKDHAIFTYTGGYDVSLSAYIIRVTTALNIVDEIIKSGGLSSGFYFEIARIENEMKINRQIMDNSAKYVEHDPRLVAEHRFETMKPNFWSRIGTVAAKRYPGVMYTFTTPLISFFGLFDINVIGLIVILFIMFMLIFNVKSKLLWFLTGTFVTAFI

SEQ ID NO: 187 - DNA, 1050 bp, synthetic construct
  source 1..1050: mol_type = other DNA
atggggggggctgccgccaggttgggggccgtgattttgtttgtcgtcatagtgggcctccatggggtccgcggcatggcggcggtgaaaactcctgttattgttgtgccagttattgatagacctccatcagaaacatttcctaatgttcatgagcatattaatgatcagaagttcgatgatgtgaaggacaatgaagttatgcaagaaaaaagagatgttgtgatagtcaatgatgatccagatcattacaaggattatgtgtttatacaatggactggaggaaatattagagatgatgacaagtatactcacttcttttcagggttttgtaacactatgtgtacagaggaaacgaaaagaaatatcgctagacatttagccctatgggattctaaattttttaccgagctagaaaataaaaatgtagaatatgtagttattatagaaaacgataacgttattgaggatattacgtttcttcgtcccgtcttgaaggcaatacatgacaagaaaatagatatcctacagatgagagaaattattacaggaaataaagttaaaaccgagcttgtaatagacaaagatcatgccatatttacatatacaggagggtatgatgttagcttatccgcctatattattagagttactacggcgctgaacatcgtagatgaaattataaagtctggaggtctatcatcgggattttattttgaaatagccagaatcgaaaacgaaatgaagattaacaggcaaataatggataactctgccaaatatgtagaacacgatcctcgtcttgttgcagaacaccgcttcgaaaccatgaaaccaaatttttggtctagaataggaacggtagctgctaaacgttatccaggagttatgtacacgtttactactccattgatttcattttttggattgtttgatattaatgttataggtttgattgtaattttgtttattatgtttatgctcatctttaatgttaaatctaaactgttatggttccttacaggaacatttgttaccgcatttatctaa

SEQ ID NO: 188 - protein, 324 aa, synthetic construct
MAAVKTPVIVVPVIDRPPSETFPNVHEHINDQKFDDVKDNEVMQEKRDVVIVNDDPDHYKDYVFIQWTGGNIRDDDKYTHFFSGFANTMATEETKRNIARHLALWDSKFFTELENKNVEYVVIIENDNVIEDITFLRPVLKAIHDKKIDILQMREIITGNKVKTELVIDKDHAIFTYTGGYDVSLSAYIIRVTTALNIVDEIIKSGGLSSGFYFEIARIENEMKINRQIMDNSAKYVEHDPRLVAEHRFETMKPNFWSRIGTVAAKRYPGVMYTFTTPLISFFGLFDINVIGLIVILFIMFMLIFNVKSKLLWFLTGTFVTAFI

SEQ ID NO: 189 - DNA, 975 bp, synthetic construct
  source 1..975: mol_type = other DNA
atggcggcggtgaaaactcctgttattgttgtgccagttattgatagacctccatcagaaacatttcctaatgttcatgagcatattaatgatcagaagttcgatgatgtgaaggacaatgaagttatgcaagaaaaaagagatgttgtgatagtcaatgatgatccagatcattacaaggattatgtgtttatacaatggactggaggaaatattagagatgatgacaagtatactcacttcttttcagggtttgctaacactatggctacagaggaaacgaaaagaaatatcgctagacatttagccctatgggattctaaattttttaccgagctagaaaataaaaatgtagaatatgtagttattatagaaaacgataacgttattgaggatattacgtttcttcgtcccgtcttgaaggcaatacatgacaagaaaatagatatcctacagatgagagaaattattacaggaaataaagttaaaaccgagcttgtaatagacaaagatcatgccatatttacatatacaggagggtatgatgttagcttatccgcctatattattagagttactacggcgctgaacatcgtagatgaaattataaagtctggaggtctatcatcgggattttattttgaaatagccagaatcgaaaacgaaatgaagattaacaggcaaataatggataactctgccaaatatgtagaacacgatcctcgtcttgttgcagaacaccgcttcgaaaccatgaaaccaaatttttggtctagaataggaacggtagctgctaaacgttatccaggagttatgtacacgtttactactccattgatttcattttttggattgtttgatattaatgttataggtttgattgtaattttgtttattatgtttatgctcatctttaatgttaaatctaaactgttatggttccttacaggaacatttgttaccgcatttatctaa

SEQ ID NO: 190 - protein, 349 aa, synthetic construct
MGGAAARLGAVILFVVIVGLHGVRGMAAVKTPVIVVPVIDRPPSETFPNVHEHINDQKFDDVKDNEVMQEKRDVVIVNDDPDHYKDYVFIQWTGGNIRDDDKYTHFFSGFANTMATEETKRNIARHLALWDSKFFTELENKNVEYVVIIENDNVIEDITFLRPVLKAIHDKKIDILQMREIITGNKVKTELVIDKDHAIFTYTGGYDVSLSAYIIRVTTALNIVDEIIKSGGLSSGFYFEIARIENEMKINRQIMDNSAKYVEHDPRLVAEHRFETMKPNFWSRIGTVAAKRYPGVMYTFTTPLISFFGLFDINVIGLIVILFIMFMLIFNVKSKLLWFLTGTFVTAFI

SEQ ID NO: 191 - DNA, 1050 bp, synthetic construct
  source 1..1050: mol_type = other DNA
atggggggggctgccgccaggttgggggccgtgattttgtttgtcgtcatagtgggcctccatggggtccgcggcatggcggcggtgaaaactcctgttattgttgtgccagttattgatagacctccatcagaaacatttcctaatgttcatgagcatattaatgatcagaagttcgatgatgtgaaggacaatgaagttatgcaagaaaaaagagatgttgtgatagtcaatgatgatccagatcattacaaggattatgtgtttatacaatggactggaggaaatattagagatgatgacaagtatactcacttcttttcagggtttgctaacactatggctacagaggaaacgaaaagaaatatcgctagacatttagccctatgggattctaaattttttaccgagctagaaaataaaaatgtagaatatgtagttattatagaaaacgataacgttattgaggatattacgtttcttcgtcccgtcttgaaggcaatacatgacaagaaaatagatatcctacagatgagagaaattattacaggaaataaagttaaaaccgagcttgtaatagacaaagatcatgccatatttacatatacaggagggtatgatgttagcttatccgcctatattattagagttactacggcgctgaacatcgtagatgaaattataaagtctggaggtctatcatcgggattttattttgaaatagccagaatcgaaaacgaaatgaagattaacaggcaaataatggataactctgccaaatatgtagaacacgatcctcgtcttgttgcagaacaccgcttcgaaaccatgaaaccaaatttttggtctagaataggaacggtagctgctaaacgttatccaggagttatgtacacgtttactactccattgatttcattttttggattgtttgatattaatgttataggtttgattgtaattttgtttattatgtttatgctcatctttaatgttaaatctaaactgttatggttccttacaggaacatttgttaccgcatttatctaa

SEQ ID NO: 192 - protein, 304 aa, synthetic construct; identical to SEQ ID NO: 41

SEQ ID NO: 193 - DNA, 915 bp, synthetic construct
  source 1..915: mol_type = other DNA
atgcctcaacaactatctcctattaatatagaaactaaaaaagcaatttctgacgcgcgattgaagacgttagacatacattataatgagtcgaaaccaaccactatccagaatactggaaaactagtaaggattaattttaaaggaggatatataagtggagggtttctccccaatgaatatgtgttatcaacaatacatatatattggggaaaggaagacgattatggatccaatcacttgatagatgtatacaaatactctggagagattaatctggttcattggaataagaaaaaatatagttcttatgaagaggcaaaaaaacacgatgatggaattatcattattgctatattcttacaagtatcagatcataaaaatgtatattttcaaaagatagtcaatcaattggattccattagatccgctaatatgtctgcgccatttgattcagtattttatctagacaatttgctacctagtacgttggattattttacatatctaggaacaactatcaaccactctgcagacgcagcatggataatttttccaacaccaataaacattcattctgatcaactatctaaattcagaacactattgtcgtcgtctaatcatgagggtaaacctcattatataacagagaattatagaaatccgtataaattgaacgacgacacgcaagtatattattctggggagattatacgagctgcaaccacctcaccagtgcgagagaactattttatgaaatggttgtctgatttgagagaggcatgtttttcatattatcaaaaatatatcgaagggaataaaacattcgcaattattgccatagtattcgtatttatactcaccgctattctctttcttatgagtcaacgatattcgcgagaaaaacaaaactag

SEQ ID NO: 194 - protein, 329 aa, synthetic construct
MGGAAARLGAVILFVVIVGLHGVRGMPQQLSPINIETKKAISDARLKTLDIHYNESKPTTIQNTGKLVRINFKGGYISGGFLPNEYVLSTIHIYWGKEDDYGSNHLIDVYKYSGEINLVHWNKKKYSSYEEAKKHDDGIIIIAIFLQVSDHKNVYFQKIVNQLDSIRSANMSAPFDSVFYLDNLLPSTLDYFTYLGTTINHSADAAWIIFPTPINIHSDQLSKFRTLLSSSNHEGKPHYITENYRNPYKLNDDTQVYYSGEIIRAATTSPVRENYFMKWLSDLREACFSYYQKYIEGNKTFAIIAIVFVFILTAILFLMSQRYSREKQN

SEQ ID NO: 195 - DNA, 990 bp, synthetic construct
  source 1..990: mol_type = other DNA
atggggggggctgccgccaggttgggggccgtgattttgtttgtcgtcatagtgggcctccatggggtccgcggcatgcctcaacaactatctcctattaatatagaaactaaaaaagcaatttctgacgcgcgattgaagacgttagacatacattataatgagtcgaaaccaaccactatccagaatactggaaaactagtaaggattaattttaaaggaggatatataagtggagggtttctccccaatgaatatgtgttatcaacaatacatatatattggggaaaggaagacgattatggatccaatcacttgatagatgtatacaaatactctggagagattaatctggttcattggaataagaaaaaatatagttcttatgaagaggcaaaaaaacacgatgatggaattatcattattgctatattcttacaagtatcagatcataaaaatgtatattttcaaaagatagtcaatcaattggattccattagatccgctaatatgtctgcgccatttgattcagtattttatctagacaatttgctacctagtacgttggattattttacatatctaggaacaactatcaaccactctgcagacgcagcatggataatttttccaacaccaataaacattcattctgatcaactatctaaattcagaacactattgtcgtcgtctaatcatgagggtaaacctcattatataacagagaattatagaaatccgtataaattgaacgacgacacgcaagtatattattctggggagattatacgagctgcaaccacctcaccagtgcgagagaactattttatgaaatggttgtctgatttgagagaggcatgtttttcatattatcaaaaatatatcgaagggaataaaacattcgcaattattgccatagtattcgtatttatactcaccgctattctctttcttatgagtcaacgatattcgcgagaaaaacaaaactag

SEQ ID NO: 196 - protein, 509 aa, synthetic construct
MANIINLWNGIVPMVQDVNVASITAFKSMIDETWDKKIEANTCISRKHRNIIHEVIRDFMKAYPKMDENRKSPLGAPMQWLTQYYILKNEYHKTMLAYDDGSLNTKFKTLNIYMITNVGQYILYIVFCIISGKNHDGTPYIYDSEITSNDKNLINDRIKYACKQILHGQLTMALRIRNKFMFIGSPMYLWFNVNGSHVYHEIYDRNVGFHNKEIGRLLYAFMYYLSISGRFLNDLALLKFTYLGESWTFSLSVPEYILYGLGYSVFDTIEKFSNDAILVYIRTNNRNGYDYAEFNKKGIVKVTEDKPDNDKRIHAIRLINYSSDVQHIHFGFRNTLIIDNECTNIQSSAENATDIGHYQDSKINIEDDDIIDDDDDDDDDDDDDDYNPKPTPIPDPHPRPPFPRHDYHKRPKLLPVEEPDPVKKDADRIRLDNHILNTLDHNLNSIGHYCCDTVAVDRLEHHIETLGQYTVILARKINMQTLLFPWPLPTVHQHAIDGSIPPHGRSTIL

SEQ ID NO: 197 - DNA, 1530 bp, synthetic construct
  source 1..1530: mol_type = other DNA
atggcgaacattataaatttatggaacggaattgtaccaatggttcaagatgttaatgttgcgagcattactgcgtttaaatctatgatagatgaaacatgggataaaaaaatcgaagcaaatacatgcatcagtagaaaacatagaaacattattcacgaagttattagggactttatgaaagcatatcctaaaatggacgagaatagaaaatctccattaggagctccaatgcaatggctaacacaatattatattttaaagaatgaatatcacaagaccatgctagcatatgatgatggatcattaaatacaaaatttaaaacattaaacatttacatgattactaacgtgggtcaatatattttatatatagtattttgtataatatctggtaagaatcatgatggtactccttatatatacgattctgaaataacgagcaatgataaaaatcttattaatgatcgtatcaagtatgcatgtaaacaaatattacacggtcaattaactatggctctgagaattagaaataaattcatgtttataggatcacccatgtatttatggtttaacgtaaacgggtcacatgtatatcacgaaatatatgatcgtaatgtcggttttcataataaagagataggtagactactatacgcattcatgtactatctatctataagcggtagatttttgaatgatttagcactattaaagtttacgtatttaggagaatcctggacatttagtttgagtgttcctgaatatatattatatggtttaggatattctgtttttgatactattgaaaaatttagcaatgatgctatactcgtttatattagaacaaacaatagaaatggttatgattatgcagagtttaataaaaaaggaattgttaaggtgacagaagataaacccgataacgataagcgaattcatgctataagactcatcaactatagtagtgatgttcaacatatacattttgggtttagaaatacgttaataatagacaatgaatgtactaatattcagtcgagtgctgaaaatgcaactgatataggacattatcaagatagcaaaataaatatcgaagatgatgatatcatagatgatgatgatgatgatgatgatgatgatgatgatgatgattataatccaaaacctactccgatacctgatcctcatcctagaccaccatttcccagacatgactatcataagaggccgaaacttcttcctgtagaagagcctgatcctgtcaaaaaagacgcggatcgtataagacttgataatcatatattaaacacattggatcataatcttaattccatcggacattattgttgtgatacagtggcagttgataggttagaacatcacattgaaacattgggacaatatacagtaatactagcaagaaagataaatatgcaaacattactgttcccatggccattacctactgtccatcaacatgcgatagatggtagtattccaccacatgggagatctacgatcttataa

SEQ ID NO: 198 - protein, 534 aa, synthetic construct
MGGAAARLGAVILFVVIVGLHGVRGMANIINLWNGIVPMVQDVNVASITAFKSMIDETWDKKIEANTCISRKHRNIIHEVIRDFMKAYPKMDENRKSPLGAPMQWLTQYYILKNEYHKTMLAYDDGSLNTKFKTLNIYMITNVGQYILYIVFCIISGKNHDGTPYIYDSEITSNDKNLINDRIKYACKQILHGQLTMALRIRNKFMFIGSPMYLWFNVNGSHVYHEIYDRNVGFHNKEIGRLLYAFMYYLSISGRFLNDLALLKFTYLGESWTFSLSVPEYILYGLGYSVFDTIEKFSNDAILVYIRTNNRNGYDYAEFNKKGIVKVTEDKPDNDKRIHAIRLINYSSDVQHIHFGFRNTLIIDNECTNIQSSAENATDIGHYQDSKINIEDDDIIDDDDDDDDDDDDDDYNPKPTPIPDPHPRPPFPRHDYHKRPKLLPVEEPDPVKKDADRIRLDNHILNTLDHNLNSIGHYCCDTVAVDRLEHHIETLGQYTVILARKINMQTLLFPWPLPTVHQHAIDGSIPPHGRSTIL

SEQ ID NO: 199 - DNA, 1605 bp, synthetic construct
  source 1..1605: mol_type = other DNA
atggggggggctgccgccaggttgggggccgtgattttgtttgtcgtcatagtgggcctccatggggtccgcggcatggcgaacattataaatttatggaacggaattgtaccaatggttcaagatgttaatgttgcgagcattactgcgtttaaatctatgatagatgaaacatgggataaaaaaatcgaagcaaatacatgcatcagtagaaaacatagaaacattattcacgaagttattagggactttatgaaagcatatcctaaaatggacgagaatagaaaatctccattaggagctccaatgcaatggctaacacaatattatattttaaagaatgaatatcacaagaccatgctagcatatgatgatggatcattaaatacaaaatttaaaacattaaacatttacatgattactaacgtgggtcaatatattttatatatagtattttgtataatatctggtaagaatcatgatggtactccttatatatacgattctgaaataacgagcaatgataaaaatcttattaatgatcgtatcaagtatgcatgtaaacaaatattacacggtcaattaactatggctctgagaattagaaataaattcatgtttataggatcacccatgtatttatggtttaacgtaaacgggtcacatgtatatcacgaaatatatgatcgtaatgtcggttttcataataaagagataggtagactactatacgcattcatgtactatctatctataagcggtagatttttgaatgatttagcactattaaagtttacgtatttaggagaatcctggacatttagtttgagtgttcctgaatatatattatatggtttaggatattctgtttttgatactattgaaaaatttagcaatgatgctatactcgtttatattagaacaaacaatagaaatggttatgattatgcagagtttaataaaaaaggaattgttaaggtgacagaagataaacccgataacgataagcgaattcatgctataagactcatcaactatagtagtgatgttcaacatatacattttgggtttagaaatacgttaataatagacaatgaatgtactaatattcagtcgagtgctgaaaatgcaactgatataggacattatcaagatagcaaaataaatatcgaagatgatgatatcatagatgatgatgatgatgatgatgatgatgatgatgatgatgattataatccaaaacctactccgatacctgatcctcatcctagaccaccatttcccagacatgactatcataagaggccgaaacttcttcctgtagaagagcctgatcctgtcaaaaaagacgcggatcgtataagacttgataatcatatattaaacacattggatcataatcttaattccatcggacattattgttgtgatacagtggcagttgataggttagaacatcacattgaaacattgggacaatatacagtaatactagcaagaaagataaatatgcaaacattactgttcccatggccattacctactgtccatcaacatgcgatagatggtagtattccaccacatgggagatctacgatcttataa

SEQ ID NO: 200 - DNA, 408 bp, synthetic construct; identical to SEQ ID NO: 167
  source 1..408: mol_type = other DNA

SEQ ID NO: 201 - DNA, 483 bp, synthetic construct
  source 1..483: mol_type = other DNA
atggggggggctgccgccaggttgggggccgtgattttgtttgtcgtcatagtgggcctccatggggtccgcggcatggggggggctgccgccaggttgggggccgtgattttgtttgtcgtcatagtgggcctccatggggtccgcggcatggacggaactcttttccccggagatgacgatcttgcaattccagcaactgaatttttctctacaaaggctgctaaaaatccagagactaaacgcgaagcaattgttaaagcctatggagacgataatgaggaaactctcaaacaacggctaactaatttggaaaaaaagattactaatataacaacaaagtttgaacaaatagaaaagtgttgtaaacgcaacgatgaagttctatttaggttggaaaatcacgctgaaactctaagagcggctatgatatctctggctaaaaagattgatgtacagactggacggcatccatatgagtaa

SEQ ID NO: 202 - DNA, 603 bp, synthetic construct
  source 1..603: mol_type = other DNA
ccccccccccccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaacc

SEQ ID NO: 203 - DNA, 2616 bp, synthetic construct
  source 1..2616: mol_type = other DNA
atggggggggctgccgccaggttgggggccgtgattttgtttgtcgtcatagtgggcctccatggggtccgcggcatggacggaactcttttccccggagatgacgatcttgcaattccagcaactgaatttttctctacaaaggctgctaaaaatccagagactaaacgcgaagcaattgttaaagcctatggagacgataatgaggaaactctcaaacaacggctaactaatttggaaaaaaagattactaatataacaacaaagtttgaacaaatagaaaagtgttgtaaacgcaacgatgaagttctatttaggttggaaaatcacgctgaaactctaagagcggctatgatatctctggctaaaaagattgatgtacagactggacggcatccatatgagtaaccccccccccccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggggggggctgccgccaggttgggggccgtgattttgtttgtcgtcatagtgggcctccatggggtccgcggcatggcgaacattataaatttatggaacggaattgtaccaatggttcaagatgttaatgttgcgagcattactgcgtttaaatctatgatagatgaaacatgggataaaaaaatcgaagcaaatacatgcatcagtagaaaacatagaaacattattcacgaagttattagggactttatgaaagcatatcctaaaatggacgagaatagaaaatctccattaggagctccaatgcaatggctaacacaatattatattttaaagaatgaatatcacaagaccatgctagcatatgatgatggatcattaaatacaaaatttaaaacattaaacatttacatgattactaacgtgggtcaatatattttatatatagtattttgtataatatctggtaagaatcatgatggtactccttatatatacgattctgaaataacgagcaatgataaaaatcttattaatgatcgtatcaagtatgcatgtaaacaaatattacacggtcaattaactatggctctgagaattagaaataaattcatgtttataggatcacccatgtatttatggtttaacgtaaacgggtcacatgtatatcacgaaatatatgatcgtaatgtcggttttcataataaagagataggtagactactatacgcattcatgtactatctatctataagcggtagatttttgaatgatttagcactattaaagtttacgtatttaggagaatcctggacatttagtttgagtgttcctgaatatatattatatggtttaggatattctgtttttgatactattgaaaaatttagcaatgatgctatactcgtttatattagaacaaacaatagaaatggttatgattatgcagagtttaataaaaaaggaattgttaaggtgacagaagataaacccgataacgataagcgaattcatgctataagactcatcaactatagtagtgatgttcaacatatacattttgggtttagaaatacgttaataatagacaatgaatgtactaatattcagtcgagtgctgaaaatgcaactgatataggacattatcaagatagcaaaataaatatcgaagatgatgatatcatagatgatgatgatgatgatgatgatgatgatgatgatgatgattataatccaaaacctactccgatacctgatcctcatcctagaccaccatttcccagacatgactatcataagaggccgaaacttcttcctgtagaagagcctgatcctgtcaaaaaagacgcggatcgtataagacttgataatcatatattaaacacattggatcataatcttaattccatcggacattattgttgtgatacagtggcagttgataggttagaacatcacattgaaacattgggacaatatacagtaatactagcaagaaagataaatatgcaaacattactgttcccatggccattacctactgtccatcaacatgcgatagatggtagtattccaccacatgggagatctacgatcttataa

SEQ ID NO: 204 - protein, 93 aa, synthetic construct
NDFDKVREALDGVNLIIHTAALVDVFGKYTDNEIMKVNYYGTQTILAACVDLGIKYLIYTSSMEAIGPNKHGNPFIGHEHTLYDISPGHVYAK

SEQ ID NO: 205 - DNA, 279 bp, synthetic construct
  source 1..279: mol_type = other DNA
aacgactttgataaggtgagagaagcgctagatggggtaaatctgattattcatacggctgctctagtggatgtatttggaaaatacactgataatgaaatcatgaaagtaaactattatggaacacagactatattggcagcttgtgtggacctaggaatcaagtatttgatctatactagtagcatggaagcaataggaccaaataaacacggtaatccattcatcggtcatgagcataccctttatgatatatcaccaggacatgtatacgcaaaa

SEQ ID NO: 206 - protein, 137 aa, synthetic construct
YPMDNVINFINQDVRYSPTFIKTIKDFVNEHLPTMYDGLNDYLHSVIIDEDLIEEYKIKSVAMFNLEYKTDIDTLTLDEQIFVEVNISYYDFRYRQFADEFRDYIMIKERRQITMQSGDRIRRFRRPMSLRSTIIKK

SEQ ID NO: 207 - DNA, 411 bp, synthetic construct
  source 1..411: mol_type = other DNA
tatcctatggacaatgttataaattttattaatcaagatgttagatatagtcccaccttcatcaagacaattaaagattttgtcaacgagcatcttccaaccatgtacgatggattaaatgattatctacattctgttattatcgacgaggacttaatagaggaatataaaattaaatccgttgccatgtttaatttggaatacaaaactgatatagatactctaacattggacgaacagatatttgtagaggtaaacatctcatattatgattttagatatagacaatttgccgatgaatttagagattacatcatgataaaagaaagaagacaaatcaccatgcaatctggtgatagaataagaaggtttagacgtcccatgtcattgagatccactatcatcaaaaag

SEQ ID NO: 208 - protein, 113 aa, synthetic construct
TLIPITDSLSFKLFVDTMSVLNDQYAKVVIFFNTIIEYIIATIYYRLTVLDNYTNVKHFVSKVLHTVMEACGVLFSHIKVNDKIEYELEEMVDKGTVPSYLYHLSINVISIIL

SEQ ID NO: 209 - DNA, 339 bp, synthetic construct
  source 1..339: mol_type = other DNA
acgcttattccaattactgattctctttcatttaagctatttgttgataccatgtctgtgttaaatgatcaatatgccaaggttgtcatcttcttcaataccatcatagaatatattatagctactatctattatagattgacagtcttggacaattatactaatgtcaaacattttgtatccaaagtgttacacactgtcatggaagcatgtggcgtactgttttcacacattaaagttaatgacaaaatagagtatgaattggaggagatggtggacaaaggtactgtaccttcttatttatatcatctgtccatcaacgtcatttcaataatattg

SEQ ID NO: 210 - protein, 97 aa, synthetic construct
FTSDYPFYVSPTEMVDVSMMSIYGEPFNHASVKESFGNFSIIELPYVGDTSMMVILPNKIDGLESIEQNLTDTNFKKWCNSLKATFIDVHIPKFKVI

SEQ ID NO: 211 - DNA, 291 bp, synthetic construct
  source 1..291: mol_type = other DNA
tttaccagtgattatcccttttacgtatctccaacggaaatggtagatgtaagtatgatgtctatttacggcgagccatttaatcacgcatctgtaaaagaatcattcggtaacttttcaatcatagaactgccatatgttggagatactagtatgatggtcattcttccaaacaagattgatggattagaatccatagaacaaaatctaacagatacaaattttaagaaatggtgtaactctctgaaagctacgtttatcgatgtgcacattcctaagtttaaggtaata

SEQ ID NO: 212 - protein, 59 aa, synthetic construct
KSSSILARRPTPKKMNIVTDSENRLKKNSYIENTNQGNILMDSIFVSTMPVETLFGSYI

SEQ ID NO: 213 - DNA, 177 bp, synthetic construct
  source 1..177: mol_type = other DNA
aaatcctcttccatactggctagacgtcctactcctaaaaaaatgaacatagttactgattcggaaaatcgtttaaagaagaatagctatatagaaaatactaatcagggaaacattctgatggatagcatcttcgtatccaccatgccggtagaaacattgtttggatcgtacata

SEQ ID NO: 214 - protein, 97 aa, synthetic construct
QFLPQIGGEAIFLIVSLCPTKKLVETGFVIPEISSNDNAKIAARILSEKRKDIIAHINTLIQYRQQLELAYYNSCMLTEFLHYCNSYADTIKESLLK

SEQ ID NO: 215 - DNA, 291 bp, synthetic construct
  source 1..291: mol_type = other DNA
caatttcttccacaaataggaggagaagctattttcttgattgtttcactttgtcctaccaagaagttagtagaaacaggattcgtcattccagaaatatcgtcaaatgataacgccaaaatcgctgctcgcattctatcagagaaacgcaaggacatcattgcgcatattaatactctaatacaatacaggcaacaattggaattggcttactataattcctgcatgttaactgagtttctgcattattgtaattcgtatgctgataccatcaaagaatcacttctaaaa

SEQ ID NO: 216 - protein, 74 aa, synthetic construct
RIPNSLHSLSAETITEKTFKDESIFINKLNGAMVEYVSTGESSILRSIEGELESLSKRERQLAKAIITPVVFYR

SEQ ID NO: 217 - DNA, 222 bp, synthetic construct
  source 1..222: mol_type = other DNA
cgcattccaaattctttgcatagtctttcggcggaaactattacagagaaaacatttaaggatgaatccatcttcatcaacaaattaaacggagccatggtagaatatgtttcgactggagaatcatccattctcagatctatagaaggtgaactagaatcactcagtaaaagagaaagacaattagccaaggcaattattacacctgtagttttctataga

SEQ ID NO: 218 - protein, 25 aa, synthetic construct
MGGAAARLGAVILFVVIVGLHGVRS

SEQ ID NO: 219 - protein, 55 aa, synthetic construct
IVGIVAGLAVLAVVVIGAVVATVMCRRKSSGGKGGSYSQAASSDSAQGSDVSLTA

SEQ ID NO: 220 - protein, 820 aa, synthetic construct
MGGAAARLGAVILFVVIVGLHGVRSNDFDKVREALDGVNLIIHTAALVDVFGKYTDNEIMKVNYYGTQTILAACVDLGIKYLIYTSSMEAIGPNKHGNPFIGHEHTLYDISPGHVYAKGGSGGGGSGGYPMDNVINFINQDVRYSPTFIKTIKDFVNEHLPTMYDGLNDYLHSVIIDEDLIEEYKIKSVAMFNLEYKTDIDTLTLDEQIFVEVNISYYDFRYRQFADEFRDYIMIKERRQITMQSGDRIRRFRRPMSLRSTIIKKGGSGGGGSGGTLIPITDSLSFKLFVDTMSVLNDQYAKVVIFFNTIIEYIIATIYYRLTVLDNYTNVKHFVSKVLHTVMEACGVLFSHIKVNDKIEYELEEMVDKGTVPSYLYHLSINVISIILGGSGGGGSGGFTSDYPFYVSPTEMVDVSMMSIYGEPFNHASVKESFGNFSIIELPYVGDTSMMVILPNKIDGLESIEQNLTDTNFKKWCNSLKATFIDVHIPKFKVIGGSGGGGSGGKSSSILARRPTPKKMNIVTDSENRLKKNSYIENTNQGNILMDSIFVSTMPVETLFGSYIGGSGGGGSGGQFLPQIGGEAIFLIVSLCPTKKLVETGFVIPEISSNDNAKIAARILSEKRKDIIAHINTLIQYRQQLELAYYNSCMLTEFLHYCNSYADTIKESLLKGGSGGGGSGGRIPNSLHSLSAETITEKTFKDESIFINKLNGAMVEYVSTGESSILRSIEGELESLSKRERQLAKAIITPVVFYRGGSGGGGSGGIVGIVAGLAVLAVVVIGAVVATVMCRRKSSGGKGGSYSQAASSDSAQGSDVSLTA

SEQ ID NO: 221 - protein, 820 aa, synthetic construct
MGGAAARLGAVILFVVIVGLHGVRSRIPNSLHSLSAETITEKTFKDESIFINKLNGAMVEYVSTGESSILRSIEGELESLSKRERQLAKAIITPVVFYRGGSGGGGSGGQFLPQIGGEAIFLIVSLCPTKKLVETGFVIPEISSNDNAKIAARILSEKRKDIIAHINTLIQYRQQLELAYYNSCMLTEFLHYCNSYADTIKESLLKGGSGGGGSGGKSSSILARRPTPKKMNIVTDSENRLKKNSYIENTNQGNILMDSIFVSTMPVETLFGSYIGGSGGGGSGGYPMDNVINFINQDVRYSPTFIKTIKDFVNEHLPTMYDGLNDYLHSVIIDEDLIEEYKIKSVAMFNLEYKTDIDTLTLDEQIFVEVNISYYDFRYRQFADEFRDYIMIKERRQITMQSGDRIRRFRRPMSLRSTIIKKGGSGGGGSGGFTSDYPFYVSPTEMVDVSMMSIYGEPFNHASVKESFGNFSIIELPYVGDTSMMVILPNKIDGLESIEQNLTDTNFKKWCNSLKATFIDVHIPKFKVIGGSGGGGSGGNDFDKVREALDGVNLIIHTAALVDVFGKYTDNEIMKVNYYGTQTILAACVDLGIKYLIYTSSMEAIGPNKHGNPFIGHEHTLYDISPGHVYAKGGSGGGGSGGTLIPITDSLSFKLFVDTMSVLNDQYAKVVIFFNTIIEYIIATIYYRLTVLDNYTNVKHFVSKVLHTVMEACGVLFSHIKVNDKIEYELEEMVDKGTVPSYLYHLSINVISIILGGSGGGGSGGIVGIVAGLAVLAVVVIGAVVATVMCRRKSSGGKGGSYSQAASSDSAQGSDVSLTA

SEQ ID NO: 222 - protein, 10 aa, synthetic construct
GGGGSGGGGS

SEQ ID NO: 223 - protein, 4 aa, synthetic construct
FRAC

SEQ ID NO: 224 - protein, 4 aa, synthetic construct
KRCF

SEQ ID NO: 225 - protein, 4 aa, synthetic construct
KKRY

SEQ ID NO: 226 - protein, 4 aa, synthetic construct
ARMA

SEQ ID NO: 227 - protein, 4 aa, synthetic construct
RRSG

SEQ ID NO: 228 - protein, 4 aa, synthetic construct
MRAC

SEQ ID NO: 229 - protein, 4 aa, synthetic construct
KMCG

SEQ ID NO: 230 - protein, 4 aa, synthetic construct
ARCA

SEQ ID NO: 231 - protein, 4 aa, synthetic construct
KKQG

SEQ ID NO: 232 - protein, 4 aa, synthetic construct
YRSY

SEQ ID NO: 233 - protein, 4 aa, synthetic construct
SFMN

SEQ ID NO: 234 - protein, 4 aa, synthetic construct
FKAA

SEQ ID NO: 235 - protein, 4 aa, synthetic construct
KRNG

SEQ ID NO: 236 - protein, 4 aa, synthetic construct
YNSF

SEQ ID NO: 237 - protein, 4 aa, synthetic construct
KKNG

SEQ ID NO: 238 - protein, 4 aa, synthetic construct
RRRG

SEQ ID NO: 239 - protein, 4 aa, synthetic construct
KRYS

SEQ ID NO: 240 - protein, 4 aa, synthetic construct
ARYA

SEQ ID NO: 241 - protein, 10 aa, synthetic construct
GGSLGGGGSG

SEQ ID NO: 242 - protein, 30 aa, synthetic construct
MDSKGSSQKGSRLLLLLVVSNLLLPQGVVG

SEQ ID NO: 243 - protein, 15 aa, synthetic construct
MWLVSLAIVTACAGA

SEQ ID NO: 244 - protein, 344 aa, synthetic construct
MDIFREIASSMKGKNVFISPASISSVLTILYYGANGSTAEQLSKYVEKEENMDKVSAQNISFKSMNKVYGRYSAVFKDSFLGKIGDKFQTVDFTDCRTIDAINKCVDIFTEGKINPLLDEPLSPDTCLLAISAVYFKAKWLMPFEKEFTSDYPFYVSPTEMVDVSMMSIYGEPFNHASVKESFGNFSIIELPYVGDTSMMVILPNKIDGLESIEQNLTDTNFKKWCNSLKATFIDVHIPKFKVIGSYNLVDTLIKLGLTDVFYSTGDYINMCNSDVSVDAMIHKTYIDVNEEYTEAAAATSVLVADCASTVTNEFCADHPFIYVIRHVDGKILFVGRYCSPTTN

SEQ ID NO: 245 - protein, 344 aa, synthetic construct; identical to SEQ ID NO: 244

SEQ ID NO: 246 - protein, 344 aa, synthetic construct; identical to SEQ ID NO: 244

SEQ ID NO: 247 - protein, 344 aa, synthetic construct; identical to SEQ ID NO: 244

SEQ ID NO: 248 - protein, 354 aa, synthetic construct
MGFCIPLRSKMLKRVSRKSSSILARRPTPKKMNIVTDSENRLKKNSYIENTNQGNILMDSIFVSTMPVETLFGSYITDDNDDYELKDLLNVTYNIKPVIVPDIKLDSVLDRDGNFRPADCFLVKLKHSDGFTKGALYLGHSAGFTATICLKNEGVSGLYIPGTSVVRSNICQGDTIVSRSSRGVQFLPQIGGEAIFLIVSLCPTKKLVETGFVIPEISSNDNAKIAARILSEKRKDIIAHINTLIQYRQQLELAYYNSCMLTEFLHYCNSYADTIKESLLKETIQKDINIIHTNITTLLNETAKVIKLVKSLVDKEDTDIVNNFITKEIKNCGGVKNRDKIVNSLSLSNLDFRL

SEQ ID NO: 249 - protein, 354 aa, synthetic construct; identical to SEQ ID NO: 248

SEQ ID NO: 250 - protein, 354 aa, synthetic construct; identical to SEQ ID NO: 248

SEQ ID NO: 251 - protein, 354 aa, synthetic construct; identical to SEQ ID NO: 248

SEQ ID NO: 252 - protein, 5 aa, synthetic construct
GGSGG

SEQ ID NO: 253 - DNA, 14 bp, synthetic construct
  source 1..14: mol_type = other DNA
gcggctctggagga

SEQ ID NO: 254 - protein, 41 aa, synthetic construct
GLIAGAVGGSLLAALVICGIVYWMRRHTQKAPKRIRLPHIR

SEQ ID NO: 255 - DNA, 123 bp, synthetic construct
  source 1..123: mol_type = other DNA
ggcctgatcgccggcgcggtgggcggcagtctcctggcagccctggtcatttgcggaattgtgtactggatgcgccgccacactcaaaaagccccaaagcgcatacgcctcccccacatccgg

SEQ ID NO: 256 - protein, 27 aa, synthetic construct
GYIPEAPRDGQAYVRKDGEWVLLSTFL

SEQ ID NO: 257 - DNA, 81 bp, synthetic construct
  source 1..81: mol_type = other DNA
ggttatattcctgaagctccaagagatgggcaagcttacgttcgtaaagatggcgaatgggtattgctttctaccttttta

SEQ ID NO: 258 - protein, 186 aa, synthetic construct
MGAAASIQTTVNTLSERISSKLEQEANASAQTKCDIEIGNFYIRQNHGCNITVKNMCSADADAQLDAVLSAATETYSGLTPEQKAYVPAMFTAALNIQTSVNTVVRDFENYVKQTCNSSAVVDNKLKIQNVIIDECYGAPGSPTNLEFINTGSSKGNCAIKALMQLTTKATTQIAPRQVAGTGVQF

SEQ ID NO: 259 - DNA, 558 bp, synthetic construct
  source 1..558: mol_type = other DNA
atgggtgccgcagcaagcatacagacgactgtgaatacactcagtgaacgtatctcgtctaaattagaacaagaagcgaacgctagtgctcaaacaaaatgtgatatagaaatcggaaatttttatatccgacaaaaccatggatgtaacatcactgttaaaaatatgtgctctgcggacgcggatgctcagttggatgctgtgttatcagccgctacagaaacatatagtggattaacaccggaacaaaaagcatacgtaccagctatgtttactgctgcgttaaacattcagacgagtgtaaacactgttgttagagattttgaaaattatgtgaaacagacttgtaattctagcgctgttgtcgataacaaattaaagatacaaaacgtaattatagatgaatgttacggagccccaggatctccaacaaatttggaatttattaatacaggatctagcaaaggaaattgtgccattaaggcgttgatgcaattgactactaaggccactactcaaatagcacctagacaagttgctggtacaggagttcagttt

SEQ ID NO: 260 - protein, 282 aa, synthetic construct
MAAVKTPVIVVPVIDRPPSETFPNVHEHINDQKFDDVKDNEVMQEKRDVVIVNDDPDHYKDYVFIQWTGGNIRDDDKYTHFFSGFCNTMCTEETKRNIARHLALWDSKFFTELENKNVEYVVIIENDNVIEDITFLRPVLKAIHDKKIDILQMREIITGNKVKTELVIDKDHAIFTYTGGYDVSLSAYIIRVTTALNIVDEIIKSGGLSSGFYFEIARIENEMKINRQIMDNSAKYVEHDPRLVAEHRFETMKPNFWSRIGTVAAKRYPGVMYTFTTPLISF

SEQ ID NO: 261 - DNA, 846 bp, synthetic construct
  source 1..846: mol_type = other DNA
atggcggcggtgaaaactcctgttattgttgtgccagttattgatagacctccatcagaaacatttcctaatgttcatgagcatattaatgatcagaagttcgatgatgtgaaggacaatgaagttatgcaagaaaaaagagatgttgtgatagtcaatgatgatccagatcattacaaggattatgtgtttatacaatggactggaggaaatattagagatgatgacaagtatactcacttcttttcagggttttgtaacactatgtgtacagaggaaacgaaaagaaatatcgctagacatttagccctatgggattctaaattttttaccgagctagaaaataaaaatgtagaatatgtagttattatagaaaacgataacgttattgaggatattacgtttcttcgtcccgtcttgaaggcaatacatgacaagaaaatagatatcctacagatgagagaaattattacaggaaataaagttaaaaccgagcttgtaatagacaaagatcatgccatatttacatatacaggagggtatgatgttagcttatccgcctatattattagagttactacggcgctgaacatcgtagatgaaattataaagtctggaggtctatcatcgggattttattttgaaatagccagaatcgaaaacgaaatgaagattaacaggcaaataatggataactctgccaaatatgtagaacacgatcctcgtcttgttgcagaacaccgcttcgaaaccatgaaaccaaatttttggtctagaataggaacggtagctgctaaacgttatccaggagttatgtacacgtttactactccattgatttcattt

SEQ ID NO: 262 - protein, 282 aa, synthetic construct
MAAVKTPVIVVPVIDRPPSETFPNVHEHINDQKFDDVKDNEVMQEKRDVVIVNDDPDHYKDYVFIQWTGGNIRDDDKYTHFFSGFANTMATEETKRNIARHLALWDSKFFTELENKNVEYVVIIENDNVIEDITFLRPVLKAIHDKKIDILQMREIITGNKVKTELVIDKDHAIFTYTGGYDVSLSAYIIRVTTALNIVDEIIKSGGLSSGFYFEIARIENEMKINRQIMDNSAKYVEHDPRLVAEHRFETMKPNFWSRIGTVAAKRYPGVMYTFTTPLISF

SEQ ID NO: 263 - DNA, 846 bp, synthetic construct
  source 1..846: mol_type = other DNA
atggcggcggtgaaaactcctgttattgttgtgccagttattgatagacctccatcagaaacatttcctaatgttcatgagcatattaatgatcagaagttcgatgatgtgaaggacaatgaagttatgcaagaaaaaagagatgttgtgatagtcaatgatgatccagatcattacaaggattatgtgtttatacaatggactggaggaaatattagagatgatgacaagtatactcacttcttttcagggtttgctaacactatggctacagaggaaacgaaaagaaatatcgctagacatttagccctatgggattctaaattttttaccgagctagaaaataaaaatgtagaatatgtagttattatagaaaacgataacgttattgaggatattacgtttcttcgtcccgtcttgaaggcaatacatgacaagaaaatagatatcctacagatgagagaaattattacaggaaataaagttaaaaccgagcttgtaatagacaaagatcatgccatatttacatatacaggagggtatgatgttagcttatccgcctatattattagagttactacggcgctgaacatcgtagatgaaattataaagtctggaggtctatcatcgggattttattttgaaatagccagaatcgaaaacgaaatgaagattaacaggcaaataatggataactctgccaaatatgtagaacacgatcctcgtcttgttgcagaacaccgcttcgaaaccatgaaaccaaatttttggtctagaataggaacggtagctgctaaacgttatccaggagttatgtacacgtttactactccattgatttcattt

SEQ ID NO: 264 - protein, 234 aa, synthetic construct
MPQQLSPINIETKKAISDARLKTLDIHYNESKPTTIQNTGKLVRINFKGGYISGGFLPNEYVLSTIHIYWGKEDDYGSNHLIDVYKYSGEINLVHWNKKKYSSYEEAKKHDDGIIIIAIFLQVSDHKNVYFQKIVNQLDSIRSANMSAPFDSVFYLDNLLPSTLDYFTYLGTTINHSADAAWIIFPTPINIHSDQLSKFRTLLSSSNHEGKPHYITENYRNPYKLNDDTQVYYS

SEQ ID NO: 265 - DNA, 702 bp, synthetic construct
  source 1..702: mol_type = other DNA
atgcctcaacaactatctcctattaatatagaaactaaaaaagcaatttctgacgcgcgattgaagacgttagacatacattataatgagtcgaaaccaaccactatccagaatactggaaaactagtaaggattaattttaaaggaggatatataagtggagggtttctccccaatgaatatgtgttatcaacaatacatatatattggggaaaggaagacgattatggatccaatcacttgatagatgtatacaaatactctggagagattaatctggttcattggaataagaaaaaatatagttcttatgaagaggcaaaaaaacacgatgatggaattatcattattgctatattcttacaagtatcagatcataaaaatgtatattttcaaaagatagtcaatcaattggattccattagatccgctaatatgtctgcgccatttgattcagtattttatctagacaatttgctacctagtacgttggattattttacatatctaggaacaactatcaaccactctgcagacgcagcatggataatttttccaacaccaataaacattcattctgatcaactatctaaattcagaacactattgtcgtcgtctaatcatgagggtaaacctcattatataacagagaattatagaaatccgtataaattgaacgacgacacgcaagtatattattct

SEQ ID NO: 266 - protein, 275 aa, synthetic construct
MPQQLSPINIETKKAISDARLKTLDIHYNESKPTTIQNTGKLVRINFKGGYISGGFLPNEYVLSTIHIYWGKEDDYGSNHLIDVYKYSGEINLVHWNKKKYSSYEEAKKHDDGIIIIAIFLQVSDHKNVYFQKIVNQLDSIRSANMSAPFDSVFYLDNLLPSTLDYFTYLGTTINHSADAAWIIFPTPINIHSDQLSKFRTLLSSSNHEGKPHYITENYRNPYKLNDDTQVYYSGEIIRAATTSPVRENYFMKWLSDLREACFSYYQKYIEGNKT

SEQ ID NO: 267 - DNA, 825 bp, synthetic construct
  source 1..825: mol_type = other DNA
atgcctcaacaactatctcctattaatatagaaactaaaaaagcaatttctgacgcgcgattgaagacgttagacatacattataatgagtcgaaaccaaccactatccagaatactggaaaactagtaaggattaattttaaaggaggatatataagtggagggtttctccccaatgaatatgtgttatcaacaatacatatatattggggaaaggaagacgattatggatccaatcacttgatagatgtatacaaatactctggagagattaatctggttcattggaataagaaaaaatatagttcttatgaagaggcaaaaaaacacgatgatggaattatcattattgctatattcttacaagtatcagatcataaaaatgtatattttcaaaagatagtcaatcaattggattccattagatccgctaatatgtctgcgccatttgattcagtattttatctagacaatttgctacctagtacgttggattattttacatatctaggaacaactatcaaccactctgcagacgcagcatggataatttttccaacaccaataaacattcattctgatcaactatctaaattcagaacactattgtcgtcgtctaatcatgagggtaaacctcattatataacagagaattatagaaatccgtataaattgaacgacgacacgcaagtatattattctggggagattatacgagctgcaaccacctcaccagtgcgagagaactattttatgaaatggttgtctgatttgagagaggcatgtttttcatattatcaaaaatatatcgaagggaataaaaca

SEQ ID NO: 268 - RNA, 110 nt, synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 269 - protein, 28 aa, synthetic construct
MKMRVMAPRTLILLLSGALALTETWAGS

SEQ ID NO: 270 - protein, 317 aa, synthetic construct
MKTISVVTLLCVLPAVVYSTCTVPTMNNAKLTSTETSFNDKQKVTFTCDQGYHSLDPNAVCETDKWKYENPCKKMCTVSDYVSELYDKPLYEVNSTMTLSCNGETKYFRCEEKNGNTSWNDTVTCPNAECQPLQLEHGSCQPVKEKYSFGEYITINCDVGYEVIGASYISCTANSWNVIPSCQQKCDMPSLSNGLISGSTFSIGGVIHLSCKSGFILTGSPSSTCIDGKWNPILPTCVRSNKEFDPVDDGPDDETDLSKLSKDVVQYEQEIESLEATYHIIIVALTIMGVIFLISVIVLVCSCDKNNDQYKFHKLLP

SEQ ID NO: 271 - protein, 110 aa, synthetic construct
MDGTLFPGDDDLAIPATEFFSTKAAKKPEAKREAIVKADEDDNEETLKQRLTNLEKKITNVTTKFEQIEKCCKRNDEVLFRLENHAETLRAAMISLAKKIDVQTGRRPYE

SEQ ID NO: 272 - RNA, 47 nt, synthetic construct
  source 1..47: mol_type = other RNA
aactagtattcttctggtccccacagactcagagagaacccgccacc

SEQ ID NO: 273 - protein, 219 aa, synthetic construct
MGGAAARLGAVILFVVIVGLHGVRGSSTTQYDHKESCNGLYYQGSCYILHSDYKSFEDAKANCAAETLPNKSDVLTTWLIDYVEDTWGSDGNPITKTTSDYQDSDVSQEVRKYFCTGGSGGGGSGGSSTTQYDHKESCNGLYYQGSCYILHSDYKSFEDAKANCAAESSTLPNKSDVLTTWLIDYVEDTWGSDGNPITKTTSDYQDSDVSQEVRKYFCT

SEQ ID NO: 274 - protein, 23 aa, synthetic construct
LSAATETYSGLTPEQKAYVPAMF

SEQ ID NO: 275 - protein, 19 aa, synthetic construct
YGAPGSPTNLEFINTGSSK

SEQ ID NO: 276 - protein, 64 aa, synthetic construct
STKAAKKPEAKREAIVKADEDDNEETLKQRLTNLEKKITNVTTKFEQIEKAAKRNDEVLFRLEN

SEQ ID NO: 277 - protein, 181 aa, synthetic construct
MMTPENDEEQTSVFSATVYGDKIQGKNKRKRVIGLCIRISMVISLLSMITMSAFLIVRQNQCMSANEAAITDSAVAVAAASSTHRKVASSTTQYDHKESCNGLYYQGSCYILHSDYKSFEDAKANCAAESSTLPNKSDVLTTWLIDYVEDTWGSDGNPITKTTSDYQDSDVSQEVRKYFCT

SEQ ID NO: 278 - protein, 31 aa, synthetic construct
KESCNGLYYQGSCYILHSDYKSFEDAKANCA

SEQ ID NO: 279 - protein, 31 aa, synthetic construct
KESCNGLYYQGSCYILHSDYQLFSDAKANCT

SEQ ID NO: 280 - protein, 31 aa, synthetic construct
QESCNGLYYQGSCYIFHSDYQLFSDAKANCA

SEQ ID NO: 281 - protein, 31 aa, synthetic construct
KESCNGLYYQGSCYIFHSDYQLFSDAKANCT

SEQ ID NO: 282 - protein, 16 aa, synthetic construct
DSGYHSLDPNAVCETD

SEQ ID NO: 283 - protein, 16 aa, synthetic construct
DQGYHSLDPNAVCETD

SEQ ID NO: 284 - protein, 16 aa, synthetic construct
DSGYYSLDPNAVCETD

SEQ ID NO: 285 - protein, 16 aa, synthetic construct
DQGYHSSDPNAVCETD

SEQ ID NO: 286 - protein, 27 aa, synthetic construct
CVRSNEEFDPVDDGPDDETDLSKLSKD

SEQ ID NO: 287 - protein, 27 aa, synthetic construct
CVRSNKEFDPVDDGPDDETDLSKLSKD

SEQ ID NO: 288 - protein, 27 aa, synthetic construct
CIRSNEEFDPVEDGPDDETDLSKLSKD

SEQ ID NO: 289 - protein, 27 aa, synthetic construct
CVRSNEEFDPIDDDPDDETDLSKISKD

SEQ ID NO: 290 - protein, 27 aa, synthetic construct
CVRSNEEFDLVDDGPDDETDLSKISKD

SEQ ID NO: 291 - protein, 27 aa, synthetic construct
CVRTNEEFDPVDDGPDDETDLSKLSKD

SEQ ID NO: 292 - protein, 317 aa, synthetic construct
MKTISVVTLLCVLPAVVYSTCTVPTMNNAKLTSTETSFNDKQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDMPSLSNGLISGSTFSIGGVIHLSCKSGFTLTGSPSSTCIDGKWNPVLPICVRTNEEFDPVDDGPDDETDLSKLSKDVVQYEQEIESLEATYHIIIVALTIMGVIFLISVIVLVCSCDKNNDQYKFHKLLP

SEQ ID NO: 293 - protein, 93 aa, synthetic construct
NDFDKVREALDGVNLIIHTAALVDVFGKYTDNEIMKVNYYGTQTILAACVDLGIKYLIYTSSMEAIGPNKHGDPFIGHEHTLYDISPGHVYAK

SEQ ID NO: 294 - protein, 93 aa, synthetic construct
  VARIANT 51: note = Any amino acid
NDFDKVREALDGVNLIIHTAALVDVFGKYTDNEIMKVNYYGTQTILAACVXLGIKYLIYTSSMEAIGPNKHGNPFIGHEHTLYDISPGHVYAK

SEQ ID NO: 295 - protein, 93 aa, synthetic construct
NDFDKVRQALDGVNLIIHTAALVDVFGKYTDNEIMKVNYYGTQTILAACVDLGIKYLIYTSSMEAIGPNKHGNPFIGHEHTLYDISPGHVYAK

SEQ ID NO: 296 - protein, 31 aa, synthetic construct
MEAIVPNKHGDPFIGHEHTLYDISPGHVYAK

SEQ ID NO: 297 - protein, 93 aa, synthetic construct
  VARIANT 51: note = Any amino acid
  VARIANT 87: note = Any amino acid
NDFDKVREALDGVNLIIHTAALVDVFGKYTDNEIMKVNYYGTQTILAACVXLGIKYLIYTSSMEAIGPNKHGNPFIGHEHTLYDISXGHVYAK

SEQ ID NO: 298 - protein, 31 aa, synthetic construct
MEAIGPNKHGDPFIGHEHTRYDISPGHVYAK

SEQ ID NO: 299 - protein, 93 aa, synthetic construct
NDFDKVREALNGVNLIIHTAALVDVFGKYTDNEIMKVNYYGTQTILAACVDLGIKYLIYTSSMEAIGPNKHGDPFIGHEHTLYDISPGHVYAK

SEQ ID NO: 300 - protein, 93 aa, synthetic construct
NDFDKVREALDGVNLIIHTAALVDVFGKYTDNEIMKVNYYGTQTILAACVDLGIKYLIYTSSMEAIGPNKHGDPFIGHEHTLYDISPGHAYAK

SEQ ID NO: 301 - protein, 93 aa, synthetic construct
NDFDKVRDALDGVNLIIHTAALVDVFGKYTDNEIMKVNYYGTQTILAACVDLGIKYLIYTSSMEAIGPNKHGDPFIGHEHTLYDISPGHVYAK

SEQ ID NO: 302 - protein, 93 aa, synthetic construct
NDFDKVREALDGVNLIIHTAALVDVFGKYTNNEIMKVNYYGTQTILAACVDLGIKYLIYTSSMEAIGPNKHGDPFIGHEHTLYDISPGHVYAK

SEQ ID NO: 303 - protein, 93 aa, synthetic construct
NDFDKVREALDGVNLIIHTAALVDVFGKYTDNEIMRVNYYGTQTILAACVDLGIKYLIYTSSMEAIGPNKHGDPFIGHEHTLYDISPGHVYAK

SEQ ID NO: 304 - protein, 93 aa, synthetic construct
NDFDKVREALDGVNLIIHTAALVDVFGKYTNNEIMRVNYYGTQTILAACVDLGIKYLIYTSSMEAIGPNKHGDPFIGHEHTLYDISPGHVYAK

SEQ ID NO: 305 - protein, 93 aa, synthetic construct
NDFDKVKEALDGVNLIIHTAALVDVFGKYTDNEIMKVNYYGTQTILAACVDLGIKYLIYTSSMEAIGPNKHGDPFIGHEYTLYDISPGHVYAK

SEQ ID NO: 306 - protein, 93 aa, synthetic construct
NDFDEVRKALDGVNLIIHTAALVDVFGKYTDNEIMKVNYYGTQTILAACVDLGIKYLIYTSSMEAIGPNKHGDPFIGHEHTLYDISPGHVYAK

SEQ ID NO: 307 - protein, 93 aa, synthetic construct
NDFDKVREALDGVNLIIHTAALVDVFGKYTDNEIMRVNYYGTQTILAACVDLGIKYLIYTSCMEAIGPNKHGDPFIGHEHTLYDISPGHVYAK

SEQ ID NO: 308 - protein, 93 aa, synthetic construct
NDFDKVREALDGVNLIIHTAALVDVFGKYTDNEIMKVNYYGTQTMLAACVDLGIKYLIYTSSMEAIGPNKHGDPFIGHEHTLYDISPGHAYAK

SEQ ID NO: 309 - protein, 21 aa, synthetic construct
NDFDKVKEELDGANLIIHTVA

SEQ ID NO: 310 - protein, 93 aa, synthetic construct
NDFDELRKALDGVNLIIHTAALVDVFGKYTDNEIMKVNYYGTQTILAACVDLGIKYLIYTSSMEAIGPNKHGDPFIGHEHTLYDISPGHVYAK

SEQ ID NO: 311 - protein, 93 aa, synthetic construct
NDFDKVTEALDGVTLSIHTATLVDVFGKYTDNEIMKVNYYGTQTILAACVDLGIKYFIYTSSMEAIGPNKHGDPFIGHEHTLYDISPGHVYEK

SEQ ID NO: 312 - protein, 93 aa, synthetic construct
NDFNKVREALDGVNLIIHTAALVDVFGKYKDREIMEINYYGTQTILAACVDLGIRYLIYTSSMEAIGPNKRGDPFVGHEHTLYDISPGHVYAK

SEQ ID NO: 313 - protein, 93 aa, synthetic construct
NDFSKMREALDGVNLIIHAAALVDVFGKYTDSEIMNVNYYGTQTVLAACVDLGIKYLIYTSSMEVIGPNKKGDPFIGNEYTPYDISPGHVYAK

SEQ ID NO: 314 - protein, 93 aa, synthetic construct
NDFDKMREAMDGVNLIIHTAALVDVFGKYTDVEIMKVNYYGTQTVLAVCVDLGIKYLIYTSSMEVIGPNKNGDPFVGHEYTPYDISPGHAYAK

SEQ ID NO: 315 - protein, 93 aa, synthetic construct
NDYSKMREVLDGVNLIIHTAALVDVFGKYTDAEIMKVNYYGTQTVLAVCVDLGIKYFIYTSSMEAVGPNKKGDPFIGHEYTQYDISPGHAYAK

SEQ ID NO: 316 - protein, 137 aa, synthetic construct
YPMDNVINFINQDVRYSPTFIKTIKDFVNEHLPTMYDGLNDYLHSVIIDEDLIEEYKIKSVAMFNLEYKTDVDTLTLDEQIFVEVNISYYDFRYRQFADEFRDYIMIKERRQITMQSGDRIRRFRRPMSLRSTIIKK

SEQ ID NO: 317 - protein, 133 aa, synthetic construct
  VARIANT 104: note = Any amino acid
YPMDNVINFINQDVRYSPTFIKTIKDFVNEHLPTMYDGLNDYLHSVIIDEDLIEEYKIKSVAMFNLEYKTDIDTLTLDEQIFVEVNISYYDFRYRQFADEFRDXIMIKERRQITMQSGDRIRRFRRPMSLRST

SEQ ID NO: 318 - protein, 137 aa, synthetic construct
YPMDNVINFINQDVRYSPTFIKTIKDFVNEHLPTMYDGLNDYLHSVIIDEDLIEEYKIKSVAMFNLEYKTDIDTLTLDEQIFVEVNISYYDFRYRQFANEFRDYIMIKERRQITMQSGDRIRRFRRPMSLRSTIIKK

SEQ ID NO: 319 - protein, 137 aa, synthetic construct
YPMDNVINFINQDVRYSPTFIKTIKDFVNKHLPTMYDGLNDYLHSVIIDEDLIEEYKIKSVAMFNLEYKTDVDTLTLDEQIFVEVNISYYDFRYRQFADEFRDYIMIKERRQITMQSGDRIRRFRRPMSLRSTIIKK

SEQ ID NO: 320 - protein, 137 aa, synthetic construct
YPMDNVINFINQDVRYSPTFIKTIKDFVNEHLPTMYDGLNDYLHSVIIDEDLIEEYKIKSVAMFNLEYKTDVDTLTLNEQIFVEVNISYYDFRYRQFADEFRDYIMIKERRQITMQSGDRIRRFRRPMSLRSTIIKK

SEQ ID NO: 321 - protein, 137 aa, synthetic construct
YPMDNVINFINQDVRYSPTFIKTIKDFVNEHLPTMYDGLNDYLHSVIVDEDLIEEYKIKSVAMFNLEYKTDVDTLTLDEQIFVEVNISYYDFRYRQFADEFRDYIMIKERRQITMQSGDRIRRFRRPMSLRSTIIKK

SEQ ID NO: 322 - protein, 137 aa, synthetic construct
YPMDNVINFINQDVRYSPTFIKTIKDFVNEHLPTMYDGLNDYLHSVIIDEDLIEEYKIKSVAMFNLEYKTDVDTLTLDEQIFVEVNISYYDFRYRQFADEFRDYIMIKERRQITMQSGDRIRRFRRPMSLRSTIVKK

SEQ ID NO: 323 - protein, 137 aa, synthetic construct
YPMDNVINFINQDVRYSPTFIKTIKDFVNEHLPTMYDGLNDYLHSVIIDEDLIEEYKIKSVAMFNLEYKTDVNTLTLDEQIFVEVNISYYDFRYRQFADEFRDYIMIKERRQITMQSGDRIRRFRRPMSLRSTIIKK

SEQ ID NO: 324 - protein, 137 aa, synthetic construct
YPMENVINFINQDVRYSPTFIKTIKDFVNEHLPTMYDGLNDYLHSVIIDEDLIEEYKIKSVAMFNLEYKTDVDTLTLDEQIFVEVNISYYDFRYRQFADEFRDYIMIKERRQITMQSGDRIRRFRRPMSLRSTIIKK

SEQ ID NO: 325 - protein, 137 aa, synthetic construct
YPMDNVINFINQDVRYSPTFIKTIKDFVNENLPTMYDGLNDYLHSVIVDEDLIEEYKIKSVAMFNLEYKTDVDTLTLDEQIFVEVNISYYDFRYRQFADEFRDYIMIKERRQITMQSGDRIRRFRRPMSLRSTIIKK

SEQ ID NO: 326 - protein, 137 aa, synthetic construct
YPMDNVINFINQDVRYSPTFIKTIKDFVNKHLPTMYDGLNDYLHSVIIDEDLIEEYKIKSVAMFNLEYKTDVNTLTLDEQIFVEVNISYYDFRYRQFADEFRDYIMIKERRQITMQSGDRIRRFRRPMSLRSTIIKK

SEQ ID NO: 327 - protein, 137 aa, synthetic construct
YPMDNVINFINQDVRYSPTFIKTIKDFVNKHLPTMYDGLNDYLHSVIIDKDLIEEYKIKSVAMFNLEYKTDVDTLTLDEQIFVEVNISYYDFRYRQFADEFRDYIMIKERRQITMQSGDRIRRFRRPMSLRSTIIKK

SEQ ID NO: 328 - protein, 137 aa, synthetic construct
YPMENVINFINQDVRYSPTFIKTIKDFVNENLPTMYDGLNDYLHSVIIDEDLIEEYKIKSVAMFNLEYKTDVDTLTLDEQIFVEVNISYYDFRYRQFADEFRDYIMIKERRQITMQSGDRIRRFRRPMSLRSTIIKK

SEQ ID NO: 329 - protein, 137 aa, synthetic construct
YPMDNVIHFINQDVRYSPTFIKTIKDFVNKHLPTMYDGLNDYLHSVIIDEDLIEEYKIKSVAMFNLEYKTDVDTLTLDEQIFVEVNISYYDFRYRQFADEFRDYIMIKERRQITMQSGDRIRRFRRPMSLRSTIIKK

SEQ ID NO: 330 - protein, 137 aa, synthetic construct
YPMDNVINFINQDVRYSPTFIKTIKDFVNKHLPTMYDGLNDYLHSVIIDEDLIEEYKIKSVAMFNLEYKTDVDTLTLDEQIFVEVNISYYDFRYRQFADEFRDYIMIKERRQITMQSGDKIRRFRRPMSLRSTIIKK

SEQ ID NO: 331 - protein, 137 aa, synthetic construct
YPMDNVINFINQDVRYSPTFIKTIKDFVNEHLPTMYDGLNDYLHSVIIDEDLIEEYKIKSVAMFNLEYKTDVDTLTLDEQIFVEVNISYYDFRYRQFADEFRDYIMLKESRQITMQSGDRIRRFRRPMSLRSTIIKK

SEQ ID NO: 332 - protein, 137 aa, synthetic construct
YPMENVINFINQDVRYSPTFIKTIKDFVNEHLPTMYDGLNDYLHSVIVDEDLIEEYKIKSVAMFNLEYKTDVDTLTLDEQIFVEVNISYYDFRYRQFADEFRDYIMIKERRQITMQSGDRIRRFRRPMSLRSTIIKK

SEQ ID NO: 333 - protein, 137 aa, synthetic construct
YPMDNVINFINQDVRYSPTFIKTIKDFVNEHLPTMYDGLNDYLHSVIVDEELIEEYKIKSVAMFNLEYKTDVDTLTLDEQIFVEVNISYYDFRYRQFADEFRDYIMIKERRQITMQSGDRIRRFRRPMSLRSTIIKK

SEQ ID NO: 334 - protein, 137 aa, synthetic construct
YPMDNVINFINQDVRYSPTFIKTIKDFVNEHLPTMYDGLNDYLHSVIVDEDLIEEYKIKSVAMFNLEYKTDVDTLTLDEQIFVEVNISYYDFRYRQFADEFRDYIMIKERRQITMQTGDRIRRFRRPMSLRSTIIKK

SEQ ID NO: 335 - protein, 137 aa, synthetic construct
YPMDNVINFINQDVRYSPTFIKTIKDFVNEHLPTMYDGLNDYLHSVIIDKDLIEEYKIKSVAMFNLEYKTDVDTLTLDEQIFVEVNISYYDFRYRQFADEFRDYIMIKERRQVTMQSGDRIRRFRRPMSLRSTVIKK

SEQ ID NO: 336 - protein, 137 aa, synthetic construct
YPMDNVINFINQDVRYSPTFIKTIKDFVNKHLPTMYDGLNDYLHSVIINEDLIEEYKIKSVAMFNLEYKTDVNTLTLDEQIFVEVNISYYDFRYRQFADEFRDYIMIKERRQITMQSGDRIRRFRRPMSLRSTIIKK

SEQ ID NO: 337 - protein, 137 aa, synthetic construct
YPMENVINFINQDVRYSPTFIKTIKDFVNENLPTMYDGLNDYLHSVIIDEDLIEEYKIKSVAMFNLEYKTDVDTLTLDEQIFVEVNISYYDFRYRQFADEFRDYIMIKERRQITMQSGDRIRRFRRPISLRSTIIKK

SEQ ID NO: 338 - protein, 137 aa, synthetic construct
YPMDNVINFINQDVRYSPTFIKTIKDFVNEHLPTMYDGLNDYLHSVIVDEDLIEEYKIKSVAMFNLEYKTDVDTLTLDEQIFVEVNISYYDFRYRQFADEFRDYIMLKESRQITMQTGDRIRRFRRPMSLRSTIIKK

SEQ ID NO: 339 - protein, 137 aa, synthetic construct
YPMENVINFINQDVRYSSTFIKTIKDFVNEHLPTMYDGLNDYLHSVIIDEDLIEEYKIKSVAMFNLEYKTDVDTLTLDEQIFVEVNISYYDFRYRQFADEFRDYIMLKESRQITMQSGDRIRRFRRPMSLRSTIIKK

SEQ ID NO: 340 - protein, 137 aa, synthetic construct
YPMENVINFINQDVRYSPTFIKTIKDFVNEHLPTMYDGLNDYLHSVIVDEDLIEEYKIKSVAMFNLEYKTDVDTLTLDEQIFVEVNISYYDFRYRQFADEFRDYIMLKESRQITMQTGDRIRRFRRPMSLRSTIIKK

SEQ ID NO: 341 - protein, 133 aa, synthetic construct
YPMENVINFINQDVRYSPTFIKTIKDFVNEHLPTMYDGLNYYLHSVIVDEDLIEEYKIKSVAMFNLEYKTDVDTLTLDEQIFVEVNISYYDFRYRQFADEFRDYIMLKESRQITMQTGDRIRRFRRPMSLRST

SEQ ID NO: 342 - protein, 137 aa, synthetic construct
YPMENVINFINQDVRYSPTFIKTIKDFVNAHLPTMYDGLNDYLHSVIVDEDLIEEYKIKSVAMFNLEYKTDVDTLTLDEQIFVEVNISYYDFRYRQFANEFRDYIMLKESRQITMQTGDRIRRFRRPMSLRSTIIKK

SEQ ID NO: 343 - protein, 113 aa, synthetic construct
  VARIANT 49: note = Any amino acid
TLIPITDSLSFKLFVDTMSVLNDQYAKVVIFFNTIIEYIIATIYYRLTXLDNYTNVKHFVSKVLHTVMEACGVLFSHIKVNDKIEYELEEMVDKGTVPSYLYHLSINVISIIL

SEQ ID NO: 344 - protein, 113 aa, synthetic construct
TLIPITDSLSFKLFVDTMSVLNDQYAKVVIFFNTIIEYIIATIYYRLTVLDNYNNVKHFVSKVLHTVMEACGVLFSHIKVNDKIEYELEEMVDKGTVPSYLYHLSINVISIIL

SEQ ID NO: 345 - protein, 113 aa, synthetic construct
TLIPITDSLSFKLSVDTMSVLNDQYAKVVIFFNTIIEYIIATIYYRLTVLDNYTNVKHFVSKVLHTVMEACGVLFSHIKVNDKIEHELEEMVDKGTVPSYLYHLSINVISIIL

SEQ ID NO: 346 - protein, 113 aa, synthetic construct
TLIPITDSLSFKLSVDTMSVLNDQYAKVVIFFNTIIEYIIATIYYRLTVLDNYTNVKHFVSKVLHTVMEACGVLFSYIKVNDKIEHELEEMVDKGTVPSYLYHLSINVISIIL

SEQ ID NO: 347 - protein, 113 aa, synthetic construct
TLIPITDSLSFKLSVDTMSVLNDQYAKVVIFFNTIIEYIIATIYYRLTVLDNYTNVKHFVSKVLHTVMEACGVVFSHIKVNDKIEHELEEMVDKGTVPSYLYHLSINVISIIL

SEQ ID NO: 348 - protein, 113 aa, synthetic construct
TLIPITDSLSFKLSVDTMSVLNDQYAKVVIFFNTIIEYIIATIYYRLAVLDNYTNVKHFVSKVLHTVMEACGVLFSHIKVNDKIEHELEEMVDKGTVPSYLYHLSINVISIIL

SEQ ID NO: 349 - protein, 113 aa, synthetic construct
TLIPITDSLSFKLSVDTMSVLNDQYAKIVIFFNTIIEYIIATIYYRLTVLDNYTNVKHFVSKVLHTVMEACGVLFSHIKVNDKIEHELEEMVDKGTVPSYLYHLSINVISIIL

SEQ ID NO: 350 - protein, 113 aa, synthetic construct
TLIPITDSLSFKLSVDTMSVLNDQYAKVVIFFNTIIEYIIATIYYRLTVLDNYTNVRHFVSKVLHTVMEACGVLFSHIKVNDKIEHELEEMVDKGTVPSYLYHLSINVISIIL

SEQ ID NO: 351 - protein, 113 aa, synthetic construct
TLIPITDSLSFKLSVDTMSVLNDQYAKVVIFFNTIIEYIIATIYYRLTVLNNYTNVKHFVSKVLHTVMEACGVLFSYIKVNDKIEHELEEMVDKGTVPSYLYHLSINVISIIL

SEQ ID NO: 352 - protein, 113 aa, synthetic construct
TLIPITDSLSFKLSVDTMSVLNDQYAKIVIFFNTIIEYIIATIYYRLTVLDNYTNVKHFVSKVLHTVMEACGVLFSYIKVNDKIEHELEEMVDKGTVPSYLYHLSINVISIIL

SEQ ID NO: 353 - protein, 113 aa, synthetic construct
TLIPITDSLSFKLSVDTMSVLNDQYAKVVIFFNTIIEYIIATIYYRLTVLDNYTNVKHFVSKVLHTVMEACGVLFSYIKVNDKIEHELEEMVDKGTVPSYLYYLSINVISIIL

SEQ ID NO: 354 - protein, 113 aa, synthetic construct
TLIPITDSLSFKLSVDTMSVLNDQYAKVVISFNTIIEYIIATIYYRLTVLDNYTNVKHFVSKVLHTVMEACGVLFSYIKVNDKIEHELEEMVDKGTVPSYLYHLSINVISIIL

SEQ ID NO: 355 - protein, 113 aa, synthetic construct
TLIPITDSLSFKLSVDTMSVLNDQYAKVVIFFNTIIEYIIATIYYRLAVLDNYTNVKHFVSKVLHTVMEACGVLFSYIKVNDKIEHELEEMVDKGTVPSYLYHLSINVISIIL

SEQ ID NO: 356 - protein, 113 aa, synthetic construct
TLIPITDSLSFKLSVDTMSVLNDQYAKVVIFFNTIIEYIIATIYYRLAVLDNYTNVKHFVSKVLHTVMEACGVLFSHIKVNDKIEHELEEMVDKGIVPSYLYHLSINVISIIL

SEQ ID NO: 357 - protein, 113 aa, synthetic construct
TLIPITDSLSFRLSVDTMSVLNDQYAKIVIFFNTIIEYIIATIYYRLTVLDNYTNVKHFVSKVLHTVMEACGVLFSHIKVNDKIEHELEEMVDKGTVPSYLYHLSINVISIIL

SEQ ID NO: 358 - protein, 113 aa, synthetic construct
TLIPITDSLSFKLSVDTMSVLNDQYAKIVIFFNTIIEYIIATIYYRLAVLDNYTNVKHFVSKVLHTVMEACGVLFSHIKVNDKIEHELEEMVDKGTVPSYLYHLSINVISIIL

SEQ ID NO: 359 - protein, 59 aa, synthetic construct
MKVNYYGTQTILAACVDLGIKYLIYTSSMEAIGPNKHGDPFIGHEHTLYDISPGHVYAK

SEQ ID NO: 360 - protein, 113 aa, synthetic construct
TLIPITDSLSFKLSVDTMSVLNDQYAKIVIFFNTIIEYIIATIYYRLTVLDNYTNVKHFVSKVLHTVMEACGVLFSHIKVNDKIEHELEEMVDKGIVPSYLYHLSINVISIIL

SEQ ID NO: 361 - protein, 113 aa, synthetic construct
TLIPITDSLSFKLSVDTMSVLNDQYAKVVIFFNTIIEYIIATIYYRLTVLNNYTNVKHFVSKVLHTVMEACGVLFSYIKVNDKIEHELEEMVDKGTVPSYLYYLSINVISIIL

SEQ ID NO: 362 - protein, 97 aa, synthetic construct
QFLPQIGGEAIFLIVSLCPTKKLVETGFIIPEISSNDNAKIAARILSEKRKDTIAHIDTLIQYRQQLELAYYNSCMLTEFLHYCNSYADTIKESLLK

SEQ ID NO: 363 - protein, 113 aa, synthetic construct
TLIPITDSLSFKLSVDTMSVLNDQYAKIVIFFNTIIEYIIATIYYRLTVLDNYTNVKHFVSKVLHTVMEACGVLFSYIKVNDKIEHELEEMVDKGTVPSYLYYLSINVISIIL

SEQ ID NO: 364 - protein, 113 aa, synthetic construct
TLIPITDSLSFKLSVDTMSVLNDQYAKIVIFFNTIIEYIIATIYYRLAVLDNYTNVKHFVSKVLHTVMEACGVLFSHIKVNDKIDHELEEMVDKGTVPSYLYHLSINVISIIL

SEQ ID NO: 365 - protein, 113 aa, synthetic construct
TLIPITDSLSFKLSVDTMSVLNDQYAKIVIFFNTIIEYIIATIYYRLTVLNNYTNVKHFVSKVLHTVMEACGVLFSYIKVNDKIEHELEEMVDKGTVPSYLYHLSINVISIIL

SEQ ID NO: 366 - protein, 113 aa, synthetic construct
TLIPITESLSFKLSVDTMSVLNDQYAKIVIFFNTIIEYIIATIYYRLTVLDNYTNVKHFVSKVLHTVMEACGVLFSHIKVNDKIEHELEEMVDKGIVPSYLYHLSINVISIIL

SEQ ID NO: 367 - protein, 113 aa, synthetic construct
TLIPITDSLSFKLSVDTMSVLNDQYAKIVIFFNTIIEYIIATIYYRLAVLDNYTNVKHFVSKVLHTVMEACGVLFSYIKVNDKIEHELEEMVDKGTVPSYLYHLSINVISIIL

SEQ ID NO: 368 - protein, 113 aa, synthetic construct
TLIPITDSLSFKLSVDTMSVLNDQYAKVVIFFNTIIEYIIATIYYRLTVLDNYATIRHFVSKVLHTVMEACGVLFSHIKVNDKIEHELEEMVDKGTVPSYLYHLSINVISIIL

SEQ ID NO: 369 - protein, 113 aa, synthetic construct
TLIPITDSLSFKLSVDTMSVLNDQYAKIVIFFNTIIEYIIATIYYRLTVLNNYTNVKHFVSKVLHTVMEACGVLFSYIKVNDKIEHELEEMVDKGTVPSYLYYLSINVISIIL

SEQ ID NO: 370 - protein, 113 aa, synthetic construct
TLIPITESLSFKLSVDAMSVLNDQYAKVVIFFNTIIEYIVATIYYRLAVLDNYTTVKHFVSKVLHTVMEACGVLFSYIKVNDKIEHELEEMVDKGTVPSYLYHLSINVISIIL

SEQ ID NO: 371 - protein, 113 aa, synthetic construct
TLIPITDSLSFKLSVDTMSVLNDQYAKVVIFFNTIIEYIVATIYYRLAVLDNYATIRHFVSKVLHTVMEACGVLFSHIKVNDKIEHELEEMVDKGTVPSYLYHLSINVISIIL

SEQ ID NO: 372 - protein, 113 aa, synthetic construct
TLIPITDSLSFKLSVDTMSVLNDQYAKIVIFFNTIIEYIVATIYYRLAVLDNYATIRHFVSKVLHTVMEACGVLFSHIKVNDKIEHELEEMVDKGTVPSYLYHLSINVISIIL

SEQ ID NO: 373 - protein, 113 aa, synthetic construct
TLIPITESLSFKLSVDTMSVLNDQYAKVVIFFNTIIEYIVATIYYRLAVLDNYATIRHFVSKVLHTVMEACGVLFSHIKVNDKIEHELEEMVDKGTVPSYLYHLSINVISIIL

SEQ ID NO: 374 - protein, 113 aa, synthetic construct
TLIPITESLSFKLSVDTMSVLNNQYAKVVIFFNTIIEYIVATIYYRLAVLDNYTTIKHFVSKVLHTVLEACGVLFSHIKVNDKIEHELEEMVDKGTVPSYLYHLSINVISIIL

SEQ ID NO: 375 - protein, 113 aa, synthetic construct
TLIPITDSLSFKLSVDTMSVLNDQYAKVVIFFNTIIEYIVATIYYRLAVLDNYATIRHFVSKVLHTVMEACGVLFSHIKVNDKIEHELEEMVDKGIVPSYLYHLSINVISIIL

SEQ ID NO: 376 - protein, 113 aa, synthetic construct
TLIPITESLSFKLSVDTMSVLNDQYAKVVIFFNTIIEYIVATIYYRLAVLDNYTTIRHFVSKVLHTVMEACGVLFSYIKVNDKIEHELEEMVDKGTVPSYLYHLSINVISIIL

SEQ ID NO: 377 - protein, 113 aa, synthetic construct
TLIPITESLSFKLSVDTMSVLNDQYAKVVIFFNTIIEYIVATIYYRLAVLDNYATIRHFVSKVLHTVMEACGVLFSHIKVNDKIEHELEEMVDKGIVPSYLYHLSINVISIIL

SEQ ID NO: 378 - protein, 113 aa, synthetic construct
TLIPITESLSFKLSVDTMSVLNDQYAKVVIFFNTIIEYIVATIYYRLAVLDNYATIRHFVSKVLHTVMEACGVLFSHIKVNDKIDHELEEMVDKGIVPSYLYHLSINVISIIL

SEQ ID NO: 379 - protein, 97 aa, synthetic construct
FTSDYPFYVSPTEMVDVSMMSIYGEPFNHASVKESFGNFSIIELPYVGDTSMMVILPNKIDGLESIEQNLTDTNFKKWCNSLEATFIDVHIPKFKVI

SEQ ID NO: 380 - protein, 97 aa, synthetic construct
FTSDYPFYVSPTEMVDVSMMSIYGEPFNHASVKESFGNFSIIELPYVGDTSMMVILPNKIDGLESIEQNLSDTNFKKWCNSLEATFIDVHIPKFKVI

SEQ ID NO: 381 - protein, 97 aa, synthetic construct
FTSDYPFYVSPTEMVDVSMMSIYGEPFNHASVKESFGNFSIIELSYVGDTSMMVILPNKIDGLESIEQNLTDTNFKKWCNSLEATFIDVHIPKFKVI

SEQ ID NO: 382 - protein, 96 aa, synthetic construct
FTSDYPFYVSPTEMVDVSMMSMYGEPFNHASVKESFGNFSIIELPYVGDTSMMVILPDKIDGLESIEQNLTDTNFKKWCNSLEATFIDVHIPKFKV

SEQ ID NO: 383 - protein, 83 aa, synthetic construct
MVDVSMMSMYGELFNHASVKESFGNFSIIELPYVGDTSMMVILPDKIDGLESIEQNLTDTNFKKWCNSLEATFIDVHIPKFKV

SEQ ID NO: 384 - protein, 96 aa, synthetic construct
FTSDYPFYVSPTEMVDVSMMSMYGEAFNHASVKESFGNFSIIELPYVGDTSMMVILPDKIDGLESIEQNLTDTNFKKWCNSLEATFIDVHIPKFKV

SEQ ID NO: 385 - protein, 96 aa, synthetic construct
FTSDYPFYVSPTEMVDVSMMSMYSEPFNHASVKESFGNFSIIELPYVGDTSMMVILPDKIDGLESIEQNLTDTNFKKWCNSLEATFIDVHIPKFKV

SEQ ID NO: 386 - protein, 96 aa, synthetic construct
FTSDYPFYVSPTEMVDVSMMSMYGEPFNHASVKESFGNFSIIELPYVGDTSMMVILPDKIDGLESIEQNLTDTNFKKWCNSLEATFIDGHIPKFKV

SEQ ID NO: 387 - protein, 96 aa, synthetic construct
FTSDYPFYVSPTEMVDVSMMSMYGKAFNHASVKESFGNFSIIELPYVGDTSMMVILPDKIDGLESIEQNLTDTNFKKWCNSLEATFIDVHIPKFKV

SEQ ID NO: 388 - protein, 96 aa, synthetic construct
FTSDYPFYVSPTEMVDVSMMSMYGELFNHASVKESFGNFSIIELPYVGDTSMMVILPDKIDGLESIEQNLTDTNFKKWCNSLDAMFIDVHIPKFKV

SEQ ID NO: 389 - protein, 96 aa, synthetic construct
FTSDYPFYVSPTEMVDVSMMSMYGEAFNHASVKESFGNFSIIELPYVGDTSMVVILPDKIDGLESIEQNLTDTNFKKWCNSLEATFIDVHIPKFKV

SEQ ID NO: 390 - protein, 96 aa, synthetic construct
FTSDYPFYVSPTEMVDVSMMSMYGEPFNHASVKESFGNFSIIELPYVGDTSMMVILPDNIDGLESIEQNLTDTNFKKWCDSLEATFIDVHIPKFKV

SEQ ID NO: 391 - protein, 96 aa, synthetic construct
FTSDYPFYVSPTEMVDVSMMSMYGKAFNHASVKESFGNFSIIELPYVGDTSMMVILPDKIDGLESIEQNLTDTNFKKWCDSLEATFIDVHIPKFKV

SEQ ID NO: 392 - protein, 96 aa, synthetic construct
FTSDYPFYVSPTEMVDVSMMSMYGEAFNHASVKESFGNFSIIELPYVGDTSMMVILPDNIDGLESIEQNLTDTNFKKWCDSLEATFIDVHIPKFKV

SEQ ID NO: 393 - protein, 96 aa, synthetic construct
FTSDYPFYVSPTEMVDVSMMSMYGKAFNHASVKESFGNFSIIELPYVGDTSIMVILPDKIDGLESIEQNLTDTNFKKWCNSLEATFIDVHIPKFKV

SEQ ID NO: 394 - protein, 96 aa, synthetic construct
FTSDYPFYVSPTEMVDVSMMSMYGEPFNHASVKESFGNFSIIELPYVGDTSMMVILPDNIDGLESIEQNLTDTNFKKWCDSMDATFIDVHIPKFKV

SEQ ID NO: 395 - protein, 96 aa, synthetic construct
FTSDYPFYVSPTEMVDVSMMSMYGESFNYASVKESFGNFSIIELPYVGNTSMMVILPDKIDGLESIKQNLTDTNFKKWCNSLEATFIDVHIPKFKV

SEQ ID NO: 396 - protein, 96 aa, synthetic construct
FTSDYPFYVSPTEMVDVSMMSMYGKAFNHASVKESFGNFSIIELPYVGDTSMMVILPDKIDGLESIEQNLTDTNFKKWCDFMEATFIDVHIPKFKV

SEQ ID NO: 397 - protein, 96 aa, synthetic construct
FTSDYPFYVSPTEMVDVSMMSMYGELFNHASVKESFGNFSIIELPYVGDTSMMVILPDKIDGLESIEQNLTDTNFKKWCDFMDAMFIDVHIPKFKV

SEQ ID NO: 398 - protein, 96 aa, synthetic construct
FTSDYPFYVSPTEMVDVSMMSMYGEAFNHASVKESFGNFSIIELPYVGDTSMMVILPDNIDGLESIEQNLTDTNFKKWCDSMDAMFIDVHIPKFKV

SEQ ID NO: 399 - protein, 96 aa, synthetic construct
FTSDYPFYVSPTEMVDVSMMSMYDEPFNHASVKESFGNFSIIELPYVGDTSMMVILPDKIDGLESIEQNLTDEKFKKWCDSLEATFINVHIPKFKV

SEQ ID NO: 400 - protein, 96 aa, synthetic construct
FTSDYPFYVSPTEMVDVSMMSMYGESFNYASVKESFGNFSIIELPYVGNTSMMVILPDKIDGLDSIKQNLTDTNFKKWCNSLEATFIDVHIPKFKV

SEQ ID NO: 401 - protein, 96 aa, synthetic construct
FTSDYPFYVSPTEMVDVSMMSMYGKAFNHASVKESFGNFSIIELPYVGDTSMMVILPDKIDGLESIEQNLTDTNFKKWCDFMDAMFIDVHIPKFKV

SEQ ID NO: 402 - protein, 96 aa, synthetic construct
FTSDYPLYVSPPEMVDVSMMSMYGKVFNHASVKESFGNFSIIELPYVGDTSMMVILPDKIDGLESIEQNLTDTNFKKWCNYLEATFIDVHSPKFKV

SEQ ID NO: 403 - protein, 96 aa, synthetic construct
FTSDYPFYVSLTEMVDVRMMSMYGESFNYASVKESFGNFSIIELPYVGNTSMMVILPDKIDGLESIKQNLTDTNFKKWCNSLEATFIDVHIPKFKV

SEQ ID NO: 404 - protein, 96 aa, synthetic construct
FTSDYPFYVSPTEMVDVSMMSMYGEAFNHASVKESFGNFSIIELPYVGDTSMVVILPDNIDGLESIEQNLTDTNFKKWCDSMDAMFIDVHIPKFKV

SEQ ID NO: 405 - protein, 96 aa, synthetic construct
LTSDYPFYVSPTEMVDVSMMSMYDEPFNHASVKESFGNFSIIELPYVGDTSMMVILPDKIDGLESIEQNLTDEKFKKWCDSLEATFINVHIPKFKV

SEQ ID NO: 406 - protein, 96 aa, synthetic construct
FTSDYPFYVSPTEMVDVSMMSMYGEAFNHASVNESFGNFSIIELPYVGDTSMMVILPDNIDGLESIEQNLTDTNFKKWCDSMDAMFIDVHIPKFKV

SEQ ID NO: 407 - protein, 96 aa, synthetic construct
FTSDYPFYVSPTEMVDVSMMSMYGELFNHAFVKESFGNFSIIELPYVGDTSMMVILPDKIDGLESIEQNLTDTNFKKWCDFMDAMFIDVHIPKFKV

SEQ ID NO: 408 - protein, 96 aa, synthetic construct
FTSDYPFYVSPTEMVDVSMMSMYGEAFNHASVKESFGNFSIIELPYVGDTSMVVILPDNIDGLESIEQNLTDTNFKKWSDSMDAMFIDVHIPKFKV

SEQ ID NO: 409 - protein, 96 aa, synthetic construct
FTSDYPFYVSPTEMVDVSMMSMYGEAFNHASVNESFGNFSIIELPYVGDTSMVVILPDNIDGLESIEQNLTDTNFKKWCDSMDAMFIDVHIPKFKV

SEQ ID NO: 410 - protein, 96 aa, synthetic construct
FTSDYPFYVSPTEMVDVSMMSMYGKAFNHASVKESFGNFSIIELPYVGNTSMMVILPDKIDGLESIEQNLTDTNFKKWCDFMDAMFIDVHIPKFKV

SEQ ID NO: 411 - protein, 96 aa, synthetic construct
FTSDYPFYVSPTEMVDVSMMSMYGEAFNHTSVKESFGNFSIIELPYVGDTSMVVILPDNIDGLESIEQNLTDTNFKKWCDSMDAMFIDVHIPKFKV

SEQ ID NO: 412 - protein, 96 aa, synthetic construct
FTSDYPFYVSPTEMVDVSMMSMYGEAFNHASVNESFGNFSIIELPYVGDTSMIVILPDNIDGLESIEQNLTDTNFKKWCDSMDAMFIDVHIPKFKV

SEQ ID NO: 413 - protein, 96 aa, synthetic construct
FTSDYPFYVSPTEMVDVSMMSMYGKAFNHASVKESFGNFSIIELPYVGDTSIMVILPDKIDGLESIEQNLTDTNFKKWCDFMDAMFIDVHIPKFKV

SEQ ID NO: 414 - protein, 96 aa, synthetic construct
FTRDHPFYVSTTDMVDVRMMSIYGEPFNHASVKESFGNFSIIELPYIGDTSMMVILPDEIDGLKSIEQNLTDENFKKWCDSMEAMFIDVHIPKFKV

SEQ ID NO: 415 - protein, 59 aa, synthetic construct
KSSSILARRPTPKKMNIVTDLENRLKKNSYIENTNQGNILMDSIFVSTMPVETLFGSYI

SEQ ID NO: 416 - protein, 14 aa, synthetic construct
KSSSILTRRPTPKK

SEQ ID NO: 417 - protein, 59 aa, synthetic construct
KSSSILTRRPTPKKMNIVTDLENRLKKNSYIENTNQGNILMDSIFVSTMPVETLFGSYI

SEQ ID NO: 418 - protein, 59 aa, synthetic construct
KSSSILARRPTPKKMNIVTDLENRLKKNSYIENTNQGNILIDSIFVSTMPVETLFGSYI

SEQ ID NO: 419 - protein, 59 aa, synthetic construct
KSSSILARRPTPKKMNIVTDLENRLKKNSYIENTNQRNILMDSIFVSTMPVETLFGSYI

SEQ ID NO: 420 - protein, 59 aa, synthetic construct
KSSSILARRPTPKKMNIVTDLENCLKKNSYIENTNQGNILMDSIFVSTMPVETLFGSYI

SEQ ID NO: 421 - protein, 59 aa, synthetic construct
KSSSILARRPTPKKMNIVTDLENSLKKNSYIENTNQGNILMDSIFVSTMPVETLFGSYI

SEQ ID NO: 422 - protein, 59 aa, synthetic construct
KSSSILARRPTPKKINIVTDLENRLKKNSYIENTNQGNILMDSIFVSTMPVETLFGSYI

SEQ ID NO: 423 - protein, 59 aa, synthetic construct
KSSSILTRRPTPKKMNIVTDLENRVKKNSYIENTNQGNILMDSIFVSTMPVETLFGSYI

SEQ ID NO: 424 - protein, 59 aa, synthetic construct
KSSSILTRRPTPKKMNVVTDLENRLKKNSYIENTNHGNILMDSIFVSTMPVETLFGSYI

SEQ ID NO: 425 - protein, 59 aa, synthetic construct
KSSSILTRRPTPKKMNIVTDLENRVKKNSYIENTNQGNILMDSIFVSTMSVETLFGSYI

SEQ ID NO: 426 - protein, 59 aa, synthetic construct
KSSSILTRRPTPKKMNIVTDLENRLRKNSYIENTNQGNILMDSIYVSTMPVETLFGSYI

SEQ ID NO: 427 - protein, 59 aa, synthetic construct
KFSSMLARRHTPKKMNIVTDLENRLKKNSYIENTNQGNILMDSIFVSTMSVETLFGSYI

SEQ ID NO: 428 - protein, 59 aa, synthetic construct
KFSSMLARRHTPKKMNIVTNLENRLKKNSYIENTNQGNILMDSIFVSTMSVETLFGSYI

SEQ ID NO: 429 - protein, 59 aa, synthetic construct
KSSSILTRRPTPKKPNIATELENRLKKNSYIENTNQGNILMDSIYVTTMPVEKLFGSYI

SEQ ID NO: 430 - protein, 59 aa, synthetic construct
KSSSILPRLPTPRKMNILTDLENRLKNNSYIENTNQRNILMDSLFVSIMPVETLFGSYI

SEQ ID NO: 431 - protein, 48 aa, synthetic construct
PTPKKINIVTELEDRIKKNSYVEKTNQGNILLDSIFVSAMRVDDLFGS

SEQ ID NO: 432 - protein, 50 aa, synthetic construct
PTPKKRNIVTELEDRIKKNSYVESTNNGNILLDSIFVSTMQVELLFDSLI

SEQ ID NO: 433 - protein, 49 aa, synthetic construct
PTPKKHNMVTELEGRIKKNSYVESTNHGNVLLDSIFVSTMQVDMFDSLI

SEQ ID NO: 434 - protein, 97 aa, synthetic construct
QFLPQIGGEAIFLIVSLCPTKKLVETGFVIPEISSNDNAKIAARILSEKRKDTIAHIDTLIQHRQQLELAYYNSCMLTEFLHYCNSYAGTIKESLLK

SEQ ID NO: 435 - protein, 97 aa, synthetic construct
QFLPQIGGEAIFLIVSLCPTKKLVETGFVIPEISSNDNAKIAARILSEKRKDTIAHINTLIQYRQQLELAYYNSCMLTEFLHYCNSYADTIKESLLK

SEQ ID NO: 436 - protein, 97 aa, synthetic construct
QFLPQIGGEAIFLIVSLCPTKKLVETGFVIPEISSNDNAKIAARILSEKRKDTIAHIDTLIQYRQQLELAYYNSCMLTEFLHYCNSYADTIKESLLK

SEQ ID NO: 437 - protein, 97 aa, synthetic construct
QFLPQIGGEAIFLIVSLCPTKKLVETGFVIPDISSNDNAKIAARILSEKRKDTIAHIDTLIQYRQQLELAYYNSCMLTEFLHYCNSYADTIKESLLK

SEQ ID NO: 438 - protein, 97 aa, synthetic construct
QFLPQIGGEAIFLIVSLCPTKKLVETGFVIPEISSNDNAKIAARILSEKRKDTIAHINTLIQYRQQLELAYYNSCMLTEFLHYCNSYAGTIKESLLK

SEQ ID NO: 439 - protein, 97 aa, synthetic construct
QFLPQIGGEAIFLIVSLCPTKKLVETGFVIPEISSNDNAKIAARILSEKRKDTIAHIDTLIQHRQQLELAYYNSCMLTEFLHYCNSYADTIKESLLK

SEQ ID NO: 440 - protein, 97 aa, synthetic construct
QFLPQIGGEAIFLIVSLCPTKKLVETGFIIPEISSNDNAKIAARILSEKRKDTIAHIDTLIQYRQQLEFAYYNSCMLTEFLHYCNSYADTIKESLLK

SEQ ID NO: 441 - protein, 97 aa, synthetic construct
QFLPQIGGEAIFLIVSLCPTKKLVETGFVIPEISSNDNAKIAARILSEKRKDTIAHIDTLIQYRQQLELAYYNSCMLTEFLHYCNSYAGTIKESLLK

SEQ ID NO: 442 - protein, 97 aa, synthetic construct
QFLPQIGGEAIFLIVSLCPTKKLVETGFIIPEISSNDNAKIAARILSEKRKDTIAHIDTLIQYRQQLELAYYNSCMLTEFLHYCNSYANTIKESLLK

SEQ ID NO: 443 - protein, 97 aa, synthetic construct
QFLPQIGGEAIFLIVSLCPTKKLVETGFVIPEISSNDNAKIAARILSEKRKDTIAHIDTLIQHRQQLEFAYYNSCMLTEFLHYCNSYADTIKESLLK

SEQ ID NO: 444 - protein, 97 aa, synthetic construct
QFLPQIGGEAIFLIVSLCPTKKLVETGFIIPDISSNDNAKIAARILSEKRKDTIAHIDTLIQYRQQLELAYYNSCMLTEFLHYCNSYADTIKEPLLK

SEQ ID NO: 445 - protein, 97 aa, synthetic construct
QFLPQIGGEAIFLIVSLCPTKKLAETGFIIPEISSNDNAKIAARILSEKRKDTIAHIDTLIQYRQQLEFAYYNSCMLTEFLHYCNSYADTIKESLLK

SEQ ID NO: 446 - protein, 97 aa, synthetic construct
QFLPQIGGEAIFLIVSLCPTKKLVETGFIIPEISSNDNAKIAARILSEKRNDTIAHIDTLIQYRQQLEFAYYNSCMLTEFLHYCNSYADTIKESLLK

SEQ ID NO: 447 - protein, 97 aa, synthetic construct
QFLPQIGGEAIFLIVSLCPTKKLVETGFVIPDISSNDNAKIAARILSEKRKDTIAHIDTLIQYRQQLELVYYNSCMLTEFLHYCNSYANTIKESLLK

SEQ ID NO: 448 - protein, 97 aa, synthetic construct
QFLPQIGGEAIFLIVSLCPTKKLVETGFIIPDISSNDNAKIAARILSEKRKDTIAHIDTLIQYRQQLELAYYNSCMLTEFLHYCNSYTDTIKEPLLK

SEQ ID NO: 449 - protein, 97 aa, synthetic construct
QFLPQIGGEAIFLIVSLCPTKKLVETGFVIPDISSNDNAKIAARILSEKHKDTIAHIDTLIQYRQQLDLAYYNSCILTEFLHYCNSYTNTIKESLLK

SEQ ID NO: 450 - protein, 97 aa, synthetic construct
QFLPQIGGEAIFLIVSLCPTKKLVETGFIIPEISSNDNAKIAARILSEKRKETIAHIDTLVQYRQQLESSYYNSCMLTEFLHYCNMYSDTIKESLLK

SEQ ID NO: 451 - protein, 97 aa, synthetic construct
QFLLQIGGEAIFLIVSLCPTKKLVETGFVIPNISSNDNAKIAARILSEKRKDTIAHIDTLIQYGQQLELAYYNSCMLTEFLHYCNLYANTIKESLLK

SEQ ID NO: 452 - protein, 97 aa, synthetic construct
QFLLQIGGEAIFLIVSLCPTKKLVETGFVIPNISSNDNAKIAARILSEKRKDTITHIDTLIQYGQQLELAYYNSCMLTEFLHYCNLYANTIKESLLK

SEQ ID NO: 453 - protein, 97 aa, synthetic construct
QFLPQIGGDAIYLIVSLCPTKKLVESGFSIPEISSNDNAKIAARILSEKRKDIILHIDTLVQYRQQLESAYYDSCMLSEFLHYCNSYSNIINESLLK

SEQ ID NO: 454 - protein, 97 aa, synthetic construct
RFLPQIGGGAIFLIVSLCPTKKSAETGFVIPERSSNDNAKIAARFLSEKGKDTIAPINTLIQYRQQLELAYYNPCMLTEFLHYCNSYAGTIKESLIK

SEQ ID NO: 455 - protein, 97 aa, synthetic construct
QFFPQIGGDAIYLIVSLCPTKKLIDSGFSIPEILSNENAKIAARILSEKRKEIIMHIDTLVQYRVQLESTYYNSCMLSEFLHYCNSYSNIINESLLK

SEQ ID NO: 456 - protein, 97 aa, synthetic construct
QFLPQIGGDAIYLIVSLCPTKKLIDSGFSIPEISSNECAKIAARILSEKRKEIIMHIDTLVQYRVQLESTYYNSCMLLEFLHYCNSYSNIINESLLK

SEQ ID NO: 457 - protein, 22 aa, synthetic construct
MLTEFLHYCNSYADTINESLLK

SEQ ID NO: 458 - protein, 22 aa, synthetic construct
MLTEFLHYCNSYAGTIKESLLK

SEQ ID NO: 459 - protein, 74 aa, synthetic construct
RIPNSLHSLSVETITEKTFKDESIFINKLNGAMVEYVSTGESSILRSIEGELESLSKRERQLAKAIITPVVFYR

SEQ ID NO: 460 - protein, 74 aa, synthetic construct
RIPNSLHSLSVETITEKTFKDESIFINKLNGAMVEYVSTGESSILRSIEGELEALSKRERQLAKAIITPVVFYR

SEQ ID NO: 461 - protein, 74 aa, synthetic construct
RIPNSLHSLSVETITEKTFKDESIFINKLNGAMVEYVSAGESSILRSIEGELESLSKRERQLAKAIITPVVFYR

SEQ ID NO: 462 - protein, 74 aa, synthetic construct
RIPNSLHSLSVETITEKTFKDESIFINKLNGSMVEYVSTGESSILRSIEGELESLSKRERQLAKAIITPVVFYR

SEQ ID NO: 463 - protein, 74 aa, synthetic construct
RIPNSLHSLSVETITEKTFKDESIFINKINGAMVEYVSTGESSILRSIEGELESLSKRERQLAKAIITPVVFYR

SEQ ID NO: 464 - protein, 74 aa, synthetic construct
RIPNSLHSLSVETITEKTFKDESIFINKLNGAMVEYVSTGESSILRSIEGELESLSKRERQLAKAIITPIVFYR

SEQ ID NO: 465 - protein, 74 aa, synthetic construct
RIPNSLHSLSVETITEKTFKDESIFINKLNGSMVEYVSTGESSILRSIEGELESLSKRERQLAKAIITPIVFYR

SEQ ID NO: 466 - protein, 74 aa, synthetic construct
HIPNSLHSLSVETITEKTFKDESIFINKLNGAMVEYVSAGESSILRSIEGELESLSKRERQLAKAIITPVVFYR

SEQ ID NO: 467 - protein, 74 aa, synthetic construct
RIPNSLHSLLVETITEKTFKDESIFINKLNGAMVEYVSTGELSILRSIEGELESLSKRERQLAKAIITPVVFYR

SEQ ID NO: 468 - protein, 74 aa, synthetic construct
RIPNSLHSLSVETITEKTFKDESIFINKLNGAMVEYVSAGESSILRSIEGELESLSKRERQLAKVIITPVVFYR

SEQ ID NO: 469 - protein, 74 aa, synthetic construct
RIPNSLHSLLVETITEKTFKDESIFINKLNGAMVEYVSTGESSILRSIEGELESLSKRERQLAKAIITPIVFYR

SEQ ID NO: 470 - protein, 74 aa, synthetic construct
RIPNSLHGLSVETITEKTFKDESIFINKINGAMVEYVSTGESSILRSIEGELESLSKRERQLAKAIITPVVFYR

SEQ ID NO: 471 - protein, 74 aa, synthetic construct
RIPNSLHSLSVETITEKTFKDESIFINKLNGAMVEYVSAGESSILRSIEGELEALSKRERQLAKAIITPVVFYR

SEQ ID NO: 472 - protein, 74 aa, synthetic construct
RIPNSLHSLSVETITEKTFKDESIFINKLNGSMVEYVSTGESSILRSIEGELEALSKRERQLAKAIITPVVFYR

SEQ ID NO: 473 - protein, 74 aa, synthetic construct
RIPNSLHSLSVETITEKTFKDESIFINKLNGAMVEYVSAGESSILRSIEGELESLSKRERQLAKAIITPIVFYR

SEQ ID NO: 474 - protein, 74 aa, synthetic construct
RIPNSLHSLSVETITEKTFKDESIFINKINGAMVEYVSTGESSILRSIEGELEALSKRERQLAKAIITPVVFYR

SEQ ID NO: 475 - protein, 74 aa, synthetic construct
RIPNSLHSLSVETITEKTFKDESIFINKINGAMVEYVSAGESSILRSIEGELEALSKRERQLAKAIITPVVFYR

SEQ ID NO: 476 - protein, 74 aa, synthetic construct
HIPNSLHSLSVETITEKTFKDESIFINKLNGAMVEYVSAGESSILRSIEGELESLSKRERQLAKAIITPIVFYR

SEQ ID NO: 477 - protein, 74 aa, synthetic construct
RIPNSLHSLSVETITEKTFKDESIFINKLNGAMVEYVSAGESSILRSIEGELEALSKRERQLAKVIITPVVFYR

SEQ ID NO: 478 - protein, 74 aa, synthetic construct
RIPNSLHSLSVETITEKTFKDESIFINKLNGAMVEYVSTGESSILRSIEGELEALGKRERQLAKVIITPVVFYR

SEQ ID NO: 479 - protein, 74 aa, synthetic construct
RIPNSLHGLSVETITEKTFKDESIFINKINGAMVEYVSTGESSILRSIEGELEALSKRERQLAKAIITPVVFYR

SEQ ID NO: 480 - protein, 74 aa, synthetic construct
RIPNSLHSLSVETITEKTFKDESIFINKLNGAMVEYVSAGESSILRSIEGELEALGKRERQLAKAIITPVVFYR

SEQ ID NO: 481 - protein, 74 aa, synthetic construct
RIPNSLHSLSVDTITEKTFKDESIFINKLNGAMVEYVSTGESSILRSIEGELEALGKRERQLAKVIITPVVFYR

SEQ ID NO: 482 - protein, 74 aa, synthetic construct
RIPNSLHSLSVETITEKTFKDESIFINKLNGAMVEYVSAGESSILRSIEGELEALGKRERQLAKVIITPVVFYR

SEQ ID NO: 483 - protein, 74 aa, synthetic construct
RIPNSLHSLSVETITEKTFKDESIFINKINGAMVEYVSTGESSILRSIEGELEALGKRERQLAKVIITPVVFYR

SEQ ID NO: 484 - protein, 74 aa, synthetic construct
RIPNSLHSLSVETITEKTFKDESIFINKLNGAMVEYVSAGESSILRSIEGELETLGKRERQLAKAIITPIVFYR

SEQ ID NO: 485 - protein, 74 aa, synthetic construct
RIPNSLHSLSVETITEKTFKDESIFINKLNGAMVEYVSAGESSILRSIEGELEALGKRERQLAKAIITPIVFYR

SEQ ID NO: 486 - protein, 74 aa, synthetic construct
RIPNSLHSLSVETITEKTFKDESIFINKINGAMVEYVSAGESSILRSIEGELEALGKRERQLAKVIITPVVFYR

SEQ ID NO: 487 - protein, 74 aa, synthetic construct
RIPNSLHSLSVETITEKTFKDESIFINKLNGAMVEYVSAGESSILRSIEGELDALGKRERQLSKVIITPVVFYR

SEQ ID NO: 488 - protein, 74 aa, synthetic construct
RIPHSLQSLSVETITERTFKDESIFINKLNGAMVEYTSSGESSILRSIEGELESLSKRERQLAKVIITPIVFYR

SEQ ID NO: 489 - protein, 74 aa, synthetic construct
RIPHSLQSLSIETITERTFKDESIFINKLNGAMVEYTSAGESSILRSIEGELESLSKRERQLAKVIITPIVFYR

SEQ ID NO: 490 - protein, 63 aa, synthetic construct
RITNSLHGLPVEPITEKTFKDESIFINKLNGAMVKYVSTGESSILRSVEDELESLSKRERQLA

SEQ ID NO: 491 - protein, 74 aa, synthetic construct
RIPHSLQSLSVETIIERTFKDESIFINKLNGAMVEYISAGEPSILKSIEGELESLSKRERQLAKVIITPIVFYR

SEQ ID NO: 492 - protein, 74 aa, synthetic construct
RIPNSLSNLTIEDLEEKTLKDGYLFINRMTGAIIEYKSECEQSIIKPIDKEFGNLLIRDKQVATVILSPVVFYR

SEQ ID NO: 493 - protein, 31 aa, synthetic construct
MEAIGPNKHGNPFIGHEHTLYDISPGHVYAK

SEQ ID NO: 494 - protein, 107 aa, synthetic construct
TLIPITDSLSFKLFVDTMSVLNDQYAKVVIFFNTIIEYIIATIYYRLTVLDNYTNVKHFVSKVLHTVMEACGVLFSHIKVNDKIEYELEEMVDKGTVPSYLYHLSIN

SEQ ID NO: 495 - protein, 19 aa, synthetic construct
MDSIFVSTMPVETLFGSYI

SEQ ID NO: 496 - protein, 31 aa, synthetic construct
MEAIGPNKHGDPFIGHEHTLYDISPGHVYAK

SEQ ID NO: 497 - protein, 137 aa, synthetic construct
YTMDNVINFINQDVRYSPTFIKTIKDFVNEHLPTMYDGLNDYLHSVIIDEDLIEEYKIKSVAMFNLEYKTDVDTLTLDEQIFVEVNISYYDFRYRQFADEFRDYIMIKERRQITMQSGDRIRRFRRPMSLRSTIIKK

SEQ ID NO: 498 - protein, 113 aa, synthetic construct
TLIPITESLSFKLSVDTMSVLNDQYAKVVIFFNTIIEYIIATIYYRLTVLDNYTNVKHFVSKVLHTVMEACGVLFSYIKVNDKIEHELEEMVDKGTVPSYLYHLSINVISIIL

SEQ ID NO: 499 - protein, 97 aa, synthetic construct
QFLPQIGGEAIFLIVSLCPTKKLVETGFIIPEISSNDNAKIAARILSEKRKDTIAHIDTLIQYRQQLELAYYNSCMLTEFLHYCNSYAGTIKESLLK

SEQ ID NO: 500 - protein, 113 aa, synthetic construct
TLIQITDSLSFKLSVDTMSVLNDQYAKVVIFFNTIIEYIIATIYYRLAVLDNYTNVKHFVSKVLHTVMEACGVLFSYIKVNDKIEHELEEMVDKGTVPSYLYHLSINVISIIL

SEQ ID NO: 501 - protein, 96 aa, synthetic construct
FTSDYPFYVSPTEMVDVSMMSMYGELFNHASVKESFGNFSIIELPYVGDTSMMVILPDKIDGLESIEQNLTDTNFKKWCNSLEATFIDVHIPKFKV

SEQ ID NO: 502 - protein, 97 aa, synthetic construct
FTSDYPFYVSPTEMVDVSMMSMYGELFNHASVKESFGNFSIIELPYVGDTSMMVILPDKIDGLESIEQNLTDTNFKKWCNSLEATFIDVHIPKFKVI

SEQ ID NO: 503 - protein, 77 aa, synthetic construct
MSMYGELFNHASVKESFGNFSIIELPYVGDTSMMVILPDKIDGLESIEQNLTDTNFKKWCNSLEATFIDVHIPKFKV

SEQ ID NO: 504 - protein, 96 aa, synthetic construct
FTSDYPFYVSLTEMVDVSMMSMYGESFNYASVKESFGNFSIIELPYVGNTSMMVILPDKIDGLESIKQNLTDTNFKKWCNSLEATFIDVHIPKFKV

SEQ ID NO: 505 - protein, 59 aa, synthetic construct
KSSSILARRPTPKKMNIVTDLENRLKKNSYIENNNQGNILMDSIFVSTMPVETLFGSYI

SEQ ID NO: 506 - protein, 59 aa, synthetic construct
KSSSILTRRPTPKKMNIVTDLENRLKKNSYIENNNQGNILMDSIFVSTMPVETLFGSYI